(19) United States
(12) Patent Application Publication
(10) Pub. No.: US 2025/0377589 A1
(43) Pub. Date: Dec. 11, 2025

(54) ONIUM SALT, CHEMICALLY AMPLIFIED RESIST COMPOSITION AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/221,932

(22) Filed: May 29, 2025

(30) Foreign Application Priority Data
Jun. 5, 2024 (JP) 2024-091395

(51) Int. Cl. G03F 7/004; C09D 137/00; C09D 141/00; G03F 7/00; G03F 7/039

(52) U.S. Cl. CPC G03F 7/0045; C09D 137/00; C09D 141/00; G03F 7/0046; G03F 7/0397; G03F 7/70033

(57) ABSTRACT

The onium salt can be used for a chemically amplified resist composition. The resist composition exhibits a high sensitivity, high resolution, having improved lithography properties such as LWR of line patterns, CDU of hole patterns, exposure latitude (EL), and depth of focus (DOF), and capable of suppressing collapse of resist patterns in photolithography using high-energy radiation such as a far ultraviolet ray, EUV, or an electron beam (EB) independent of whether it is of positive or negative tone, and to provide a pattern forming process. The onium salt having the formula (1).

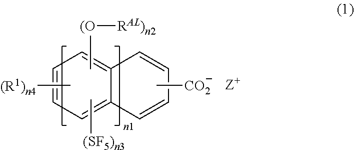

### CROSS-REFERENCE TO RELATED APPLICATION

This non-provisional application claims priority under 35 U.S.C. § 119 (a) on Patent Application No. 2024-091395 filed in Japan on Jun. 5, 2024, the entire contents of which are hereby incorporated by reference.

### TECHNICAL FIELD

This invention relates to an onium salt, a chemically amplified resist composition, and a pattern forming process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using extreme ultraviolet ray (EUV) of wavelength 13.5 nm has been implemented in a mass scale. Studies are made on the application of EUV lithography to 3-nm node devices of the next generation and 2 nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post exposure bake (PEB) fails, resulting in drastic reductions of sensitivity and contrast.

A triangular tradeoff relationship among sensitivity, resolution, and line width roughness of line patterns (LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or an iodonium salt having a polymerizable unsaturated bond, capable of generating a specific sulfonic acid. Patent Document 2 discloses a sulfonium salt having a sulfonic acid directly attached to the backbone.

An acid labile group used in a (meth)acrylate polymer for an ArF resist composition undergoes a deprotection reaction when a photoacid generator capable of generating a sulfonic acid which is substituted at α-position with a fluorine atom is used, but does not undergo a deprotection reaction when an acid generator capable of generating a sulfonic acid which is not substituted at α-position with a fluorine atom or a carboxylic acid is used. When a sulfonium salt or an iodonium salt capable of generating a sulfonic acid which is substituted at α-position with a fluorine atom is mixed with a sulfonium salt or an iodonium salt capable of generating a sulfonic acid which is not substituted at α-position with a fluorine atom, the sulfonium salt or the iodonium salt capable of generating a sulfonic acid which is not substituted at α-position with a fluorine atom undergoes ion exchange with the sulfonic acid which is substituted at α-position with a fluorine atom. Through the ion exchange, the sulfonic acid which is substituted at α-position with a fluorine atom once generated upon light exposure is converted back to the sulfonium salt or the iodonium salt. Then the sulfonium salt or the iodonium salt of the sulfonic acid which is not substituted at α-position with a fluorine atom or the carboxylic acid functions as a quencher. Patent Document 3 discloses a resist composition containing a sulfonium salt or an iodonium salt capable of generating a carboxylic acid as the quencher.

Sulfonium salt type quenchers capable of generating a carboxylic acid are known. Proposed thus far are sulfonium salts of salicylic acid and β-hydroxycarboxylic acid (Patent Document 4), salicylic acid derivatives (Patent Documents 5 and 6), fluorosalicylic acids (Patent Document 7), and hydroxynaphthoeic acid (Patent Document 8). In particular, salicylic acid is quite effective for suppressing acid diffusion due to the intramolecular hydrogen bond of a carboxy group and a hydroxy group.

It is pointed out that the agglomeration of a quencher causes degradation of the dimensional uniformity or CDU of resist patterns. It is thus expected that the dimensional uniformity of resist patterns after development is improved by preventing the quencher from agglomerating in the resist film, for achieving a uniform distribution of the quencher.

In connection with the demand for further miniaturization, there is a problem that particularly in alkaline development of a positive resist film in a developer, the resist film is swollen with the developer so that pattern collapse may occur upon small-size pattern formation. Patent Document 9 proposes an onium salt type quencher of an aromatic carboxylic acid generation type in which a phenolic hydroxy group is protected with a protective group and a fluorine atom-containing substituent is introduced in the vicinity. Thereby dissolution contrast and swelling with an alkaline developer are improved to some extent, but there is still room for improvement. To solve the problem associated with miniaturization, the development of a material for a new resist composition is important.

It is desired to develop an onium salt type quencher having good sensitivity, fully controlled acid diffusion, excellent solvent solubility, and effective in prevention of pattern collapse.

### CITATION LIST

Patent Document 1: JP-A 2006-045311
Patent Document 2: JP-A 2006-178317
Patent Document 3: JP-A 2007-114431
Patent Document 4: WO 2018/159560 A
Patent Document 5: JP-A 2020-203984
Patent Document 6: JP-A 2020-91404
Patent Document 7: JP-A 2020-91312
Patent Document 8: JP-A 2019-120760
Patent Document 9: JP-A 2024-10543
Non Patent Document 1: SPIE Vol. 6520 65203L-1 (2007)

### SUMMARY OF THE INVENTION

The invention has been made in view of the above circumstances, and an object of the invention is to provide an onium salt used for a chemically amplified resist composition and a chemically amplified resist composition, the resist composition exhibiting a high sensitivity, high resolution, having improved lithography properties such as LWR of line patterns, CDU of hole patterns, exposure latitude (EL), and depth of focus (DOF), and capable of suppressing collapse of resist patterns in photolithography using high-energy radiation such as a far ultraviolet ray, EUV, or an electron beam (EB) independent of whether it is of positive or negative tone, and to provide a pattern forming process.

As a result of intensive studies to achieve the above object, the inventor has found that a resist composition containing, as a quencher, an onium salt containing an aromatic carboxylic acid anion having an aromatic ring in which a hydroxy group protected with an acid labile group and a pentafluorosulfanyl group (—SF⁵ group) are attached to mutually adjacent carbon atoms is excellent in sensitivity and resolution of a resist film, has improved lithography properties such as LWR, CDU, exposure latitude (EL), and depth of focus (DOF), further suppresses swelling during development, and is extremely effective in precise fine processing, and has completed the invention.

That is, the invention provides an onium salt, a chemically amplified resist composition, and a pattern forming process described below.

1. An onium salt having the formula (1):

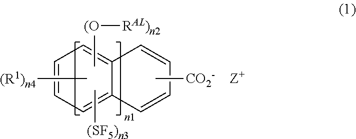

wherein n1 is 0 or 1, n2 is 1, 2, or 3, n3 is 1 or 2, n4 is 0, 1, or 2, meeting 2≤n2+n3+n4≤5 when n1 is 0, and 2≤n2+n3+n4≤7 when n1 is 1,
R^{AL} is an acid labile group, at least one —SFs group and at least one —O—R^{AL} are attached to mutually adjacent carbon atoms,
R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, two R¹'s may be identical or different and two R¹'s may bond together to form a ring with the carbon atoms to which they are attached, and
Z⁺ is an onium cation.

2. The onium salt of 1 having the formula (1A):

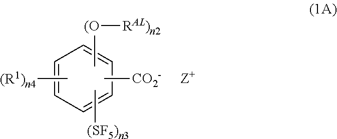

wherein n2 to n4, R¹, R^{AL}, and Z⁺ are as defined above.

3. The onium salt of 1 or 2, wherein the acid labile group has the formula (AL-1) or (AL-2):

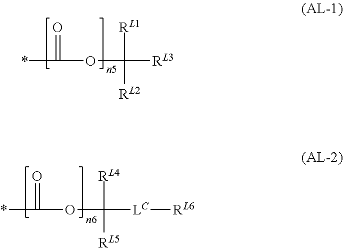

wherein n5 is 0 or 1, n6 is 0 or 1,
R^{L1}, R^{L2}, and R¹³ are each independently a C₁-C₁₂ hydrocarbyl group, some —CH₂—in the hydrocarbyl group may be replaced by —O— or —S—, when the hydrocarbyl group contains an aromatic ring, some or all hydrogen atoms in the aromatic ring may be substituted with a halogen atom, a cyano group, a nitro group, a C₁-C₄ alkyl group which may contain a halogen atom, or a C₁-C₄ alkoxy group which may contain a halogen atom,
R^{L1} and R¹² may bond together to form a ring, some —CH₂— in the ring may be replaced by —O— or —S—,
   R^{L4} and R¹⁵ are each independently a hydrogen atom or a C₁-C₁₀ hydrocarbyl group, R^{L6} is a C₁-C₂₀ hydrocarbyl group, some —CH₂— in the hydrocarbyl group may be replaced by —O— or —S—, R^{L5} and R^{L6} may bond together to form a C₃-C₂₀ heterocyclic group with the carbon atom and L^{C} to which they are attached, some —CH₂— in the heterocyclic group may be replaced by —O— or —S—,
L^{C} is —O— or —S—, and
designates a point of attachment to the adjacent —O—.

4. The onium salt of any one of 1 to 3, wherein Z⁺ is a sulfonium cation having the formula (Z-1), an iodonium cation having the formula (Z-2), or an ammonium cation having the formula (Z-3):

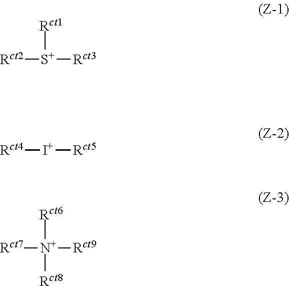

wherein R^{ct1} to R^{ct9} are each independently a halogen atom or a C₁-C₃₀ hydrocarbyl group which may contain a heteroatom, and R^{ct1} and R^{ct2} may bond together to form a ring with the sulfur atom to which they are attached, and any two of R^{ct6} to R^{ct9} may bond together to form a ring with the nitrogen atom to which they are attached.

5. The onium salt of any one of 1 to 3, wherein Z⁺ is a sulfonium cation having the formula (Z-4):

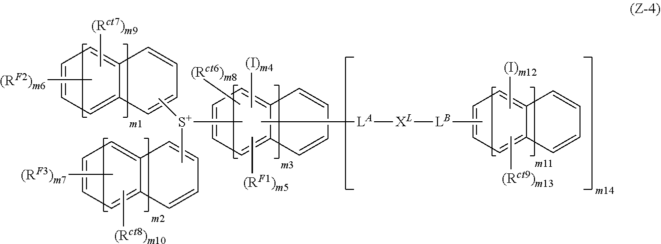

wherein m1 is 0 or 1, m2 is 0 or 1, m3 is 0 or 1, m4 is 0, 1, 2, 3, or 4, m5 is 0, 1, 2, 3, or 4, m6 is 0, 1, 2, 3, 4, 5, or 6, m7 is 0, 1, 2, 3, 4, 5, or 6, m8 is 0, 1, or 2, m9 is 0, 1, or 2, m10 is 0, 1, or 2, m11 is 0 or 1, m12 is 0, 1, 2, 3, or 4, m13 is 0, 1, or 2, m14 is 0, 1, or 2, meeting 0≤m6+m9≤4 when m1 is 0, 0≤m6+m9≤6 when m1 is 1, 0≤m7+m10≤4 when m2 is 0, 0≤m7+m10≤6 when m2 is 1, 1≤m4+m5+m8+m14≤4 when m3 is 0, 1≤m4+m5+m8+m14≤6 when m3 is 1, 0≤m12+m13≤4 when m11 is 0, and 0≤m12+m13≤6 when m11 is 1, and m4+m12≥1,
R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when m5 is 2 or more, a plurality of R^{F1}'s may be identical or different, when m6 is 2 or more, a plurality of R^{F2}'s may be identical or different, when m7 is 2 or more, a plurality of R^{F3}'s may be identical or different,
R^{ct6} to R^{ct9} are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m8 is 2, two R^{ct6}'s may be identical or different and two R^{ct6}'s may bond together to form a ring with the carbon atom to which they are attached, when m9 is 2, two Rot7's may be identical or different and two R^{ct7}'s may bond together to form a ring with the carbon atom to which they are attached, when m10 is 2, two R^{ct8}'s may be identical or different and two R^{ct8}'s may bond together to form a ring with the carbon atom to which they are attached, when m13 is 2, two R^{ct9}'s may be identical or different and two R^{ct9}'s may bond together to form a ring with the carbon atom to which they are attached,
the aromatic rings directly attached to S⁺ in the sulfonium cation may bond together to form a ring with S+,
L^{A} and L^{B} are each independently a single bond, an ether bond, an ester bond, an amide bond, a sulfonate ester bond, a sulfonamide bond, a carbonate bond, or a carbamate bond, and
X^{L} is a single bond or a C₁-C₄₀ hydrocarbylene group which may contain a heteroatom.

6. A quencher containing the onium salt of any one of 1 to 5.

7. A chemically amplified resist composition containing the quencher of 6.

8. The chemically amplified resist composition of 7, further containing a base polymer.

9. The chemically amplified resist composition of 8, wherein the base polymer contains at least one repeat unit selected from a repeat unit having the formula (a1) and a repeat unit having the formula (a2):

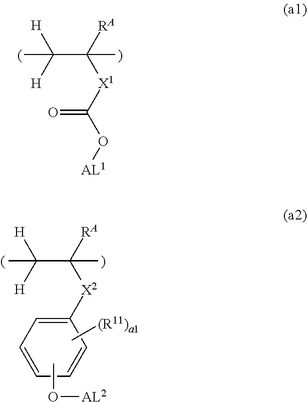

wherein R^{A}'s are each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom in the backbone,
R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2, 3, or 4, R¹¹'s may be identical or different,
AL¹ and AL² are each independently an acid labile group, and
a1 is 0, 1, 2, 3, or 4.

10. The chemically amplified resist composition of 8 or 9, wherein the base polymer contains a repeat unit having the formula (a3):

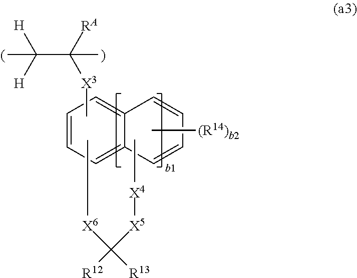

wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom in the backbone,
X⁴ is a single bond, a C₁-C₄ aliphatic hydrocarbylene group, a carbonyl group, a sulfonyl group, or a group obtained by combining these groups,
X⁵ and X⁶ are each independently an oxygen atom or a sulfur atom, X⁴ and X⁶ are attached to the adjacent carbon atoms on the aromatic rings,
R¹² and R¹³ are each independently a hydrogen atom or a C₁-C₂₀ hydrocarbyl group which may contain a heteroatom, and R¹² and R¹³ may bond together to form a ring with the carbon atom to which they are attached,
R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A}) (R^{14B}), R^{14A} and R^{14B} are each independently a hydrogen atom or a C₁-C₆ hydrocarbyl group, when b2 is 2 or more, a plurality of R¹⁴'s may be identical or different, and a plurality of R¹⁴'s may bond together to form a ring with the carbon atom on the aromatic ring to which they are attached.

11. The chemically amplified resist composition of 8, wherein the base polymer does not contain an acid labile group.

12. The chemically amplified resist composition of any one of 8 to 11, wherein the base polymer contains at least one repeat unit selected from a repeat unit having the formula (b1) and a repeat unit having the formula (b2):

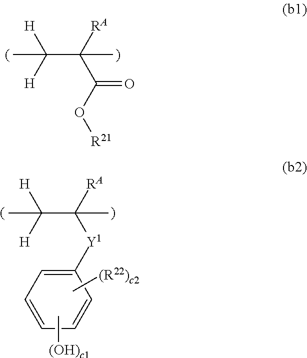

wherein R^{A}'s are each independently a hydrogen atom, a fluorine atom, a methyl group, or a trifluoromethyl group,
Y¹ is a single bond or *—C(═O)—O—, * designates a point of attachment to the carbon atom in the backbone,
R²¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2, 3, or 4, a plurality of R²²'s may be identical or different,
c1 is 1, 2, 3 or 4, and c2 is 0, 1, 2, 3, or 4, meeting 1≤c1+c2≤5.

13. The chemically amplified resist composition of any one of 8 to 12, wherein the base polymer contains at least one repeat unit selected from a repeat unit having the formula (c1), a repeat unit having the formula (c2), a repeat unit having the formula (c3), a repeat unit having the formula (c4), and a repeat unit having the formula (c5):

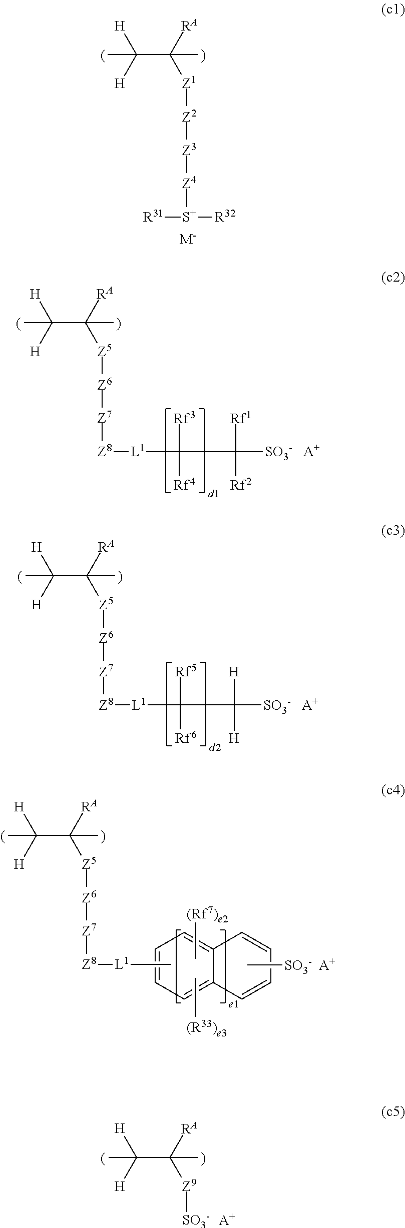

wherein d1 and d2 are each independently 0, 1, 2, or 3, e1 is 0 or 1, e2 is 0, 1, 2, 3, or 4, and e3 is 0, 1, 2, 3, or 4, meeting 0≤e2+e3≤4 when e1 is 0, and 0≤e2+e3≤6 when e1 is 1,
R^{A}'s are each independently a hydrogen atom, a fluorine atom, a methyl group, or a trifluoromethyl group,
Z¹ is a single bond or a phenylene group which may have a substituent,
Z² is a single bond, **—C(═O)—O—Z²¹—, **—C(═O)—NH—Z²¹—, or **—O—Z²¹—, Z²¹ is a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z³ is a single bond, an ether bond, an ester bond, an amide bond, a sulfonate ester bond, a sulfonamide bond, a carbonate bond, or a carbamate bond,
Z⁴ is a single bond, or a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z⁵'s are each independently a single bond, a phenylene group which may have a substituent, a naphthylene group, or *—C(═O)—O—Z⁵¹—, Z⁵¹ is a C₁-C₁₀ aliphatic hydrocarbylene group, a phenylene group, or a naphthylene group, and the aliphatic hydrocarbylene group may contain a halogen atom, a hydroxy group, an ether bond, an ester bond, or a lactone ring,
Z⁶ is a single bond, an ether bond, an ester bond, an amide bond, a sulfonate ester bond, a sulfonamide bond, a carbonate bond, or a carbamate bond,
Z⁷'s are each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
Z⁸'s are each independently a single bond, ****—Z⁸¹—C(═O)—O—, ****—C(═O)—NH—Z⁸¹—, or ****—O—Z⁸¹—, Z⁸¹ is a C₁-C₂₀ hydrocarbylene group which may contain a heteroatom,
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—N(H)—Z⁹¹—, or *—O—Z⁹¹—, Z⁹¹ is a C₁-C₆ aliphatic hydrocarbylene group, a phenylene group, a fluorinated phenylene group, or a phenylene group substituted with a trifluoromethyl group, and may contain a carbonyl group, an ester bond, an ether bond, or a hydroxy group,
* designates a point of attachment to the carbon atom in the backbone, ** designates a point of attachment to Z¹, *** designates a point of attachment to Z⁶, and **** designates a point of attachment to Z′,
L¹ is a single bond, an ether bond, an ester bond, a carbonyl group, a sulfonate ester bond, a sulfonamide bond, a carbonate bond, or a carbamate bond,
Rf¹ and Rf² are each independently a fluorine atom or a C₁-C₆ fluorinated saturated hydrocarbyl group,
Rf³ and Rf⁴ are each independently a hydrogen atom, a fluorine atom, or a C₁-C₆ fluorinated saturated hydrocarbyl group,
Rf⁵ and Rf⁶ are each independently a hydrogen atom, a fluorine atom, or a C₁-C₆ fluorinated saturated hydrocarbyl group, excluding that all Rf⁵ and Rf⁶ are a hydrogen atom at the same time,
Rf⁷ is a fluorine atom, a C₁-C₆ fluorinated alkyl group, a C₁-C₆ fluorinated alkoxy group, or a C₁-C₆ fluorinated alkylthio group,
R³¹ and R³² are each independently a C₁-C₂₀ hydrocarbyl group which may contain a heteroatom, and R³¹ and R³² may bond together to form a ring with the sulfur atom to which they are attached,
R³³ is a halogen atom other than a fluorine atom, or a C₁-C₂₀ hydrocarbyl group which may contain a heteroatom, when e3 is 2, 3, or 4, a plurality of R³³'s may be identical or different, and a plurality of R³³'s may bond together to form a ring with the carbon atom to which they are attached,
M⁻ is a non-nucleophilic counter ion, and
A⁺ is an onium cation.

14. The chemically amplified resist composition of any one of 7 to 13, further containing an organic solvent.

15. The chemically amplified resist composition of any one of 7 to 14, further containing an acid generator that generates an acid having a pKa≤−2.0.

16. The chemically amplified resist composition of any one of 7 to 15, further containing a quencher other than the quencher of 6.

17. The chemically amplified resist composition of any one of 7 to 16, further containing a surfactant.

18. A pattern forming process including the steps of forming a resist film on a substrate using the chemically amplified resist composition of any one of 7 to 17, exposing the resist film to high-energy radiation, and developing the exposed resist film using a developer.

19. The pattern forming process of 18, wherein the high-energy radiation is KrF excimer laser light, ArF excimer laser light, an electron beam (EB), or EUV with a wavelength of 3 to 15 nm.

### Advantageous Effects of the Invention

The onium salt of the invention functions well as a quencher (acid diffusion control agent) in a resist composition, exhibits a high sensitivity, and an excellent dissolution contrast, and as a result, LWR and CDU are improved, and a pattern profile with high resolution and excellent rectangularity can be constructed. In addition, the invention provides a resist composition that can suppress swelling of a resist pattern during alkaline development, can form a pattern resistant to collapse, and is excellent in fine pattern formation.

### DESCRIPTION OF THE PREFERRED EMBODIMENT

### Onium Salt

The onium salt of the invention has the formula (1).

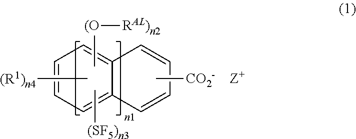

In the formula (1), n1 is 0 or 1. The relevant structure is a benzene ring when n1 is 0 and a naphthalene ring when n1 is 1, and from the aspect of solvent solubility, a benzene ring when n1 is 0 is preferred. n2 is 1, 2, or 3. From the aspect of raw material procurement, n2 is preferably 1 or 2. n3 is 1 or 2. From the aspect of raw material procurement, n3 is preferably 1. n4 is 0, 1, or 2. From the aspect of raw material procurement, n4 is preferably 0 or 1. These subscripts meet 2≤n2+n3+n4≤5 when n1 is 0, and 2≤n2+n3+n4≤7 when n1 is 1.

In the formula (1), R^{AL} is an acid labile group. The acid labile group is preferably a group having the formula (AL-1) or (AL-2).

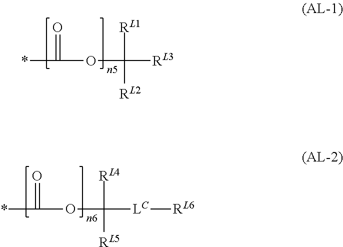

In the formula (AL-1), n5 is 0 or 1, R^{L1}, R^{L2}, and R¹³ are each independently a C₁-C₁₂ hydrocarbyl group, some —CH₂— in the hydrocarbyl group may be replaced by —O— or —S—, when the hydrocarbyl group contains an aromatic ring, some or all hydrogen atoms in the aromatic ring may be substituted with a halogen atom, a cyano group, a nitro group, a C₁-C₄ alkyl group which may contain a halogen atom, or a C₁-C₄ alkoxy group which may contain a halogen atom,
and R^{L1} and R^{L2} may bond together to form a ring with the carbon atom to which they are attached, some —CH₂— in the ring may be replaced by —O— or —S—.

In the formula (AL-2), n6 is 0 or 1, R¹⁴ and R¹⁵ are each independently a hydrogen atom or a C₁-C₁₀ hydrocarbyl group, R¹⁶ is a C₁-C₂₀ hydrocarbyl group, some —CH₂— in the hydrocarbyl group may be replaced by —O— or —S—, R^{L5} and R^{L6} may bond together to form a C₃-C₂₀ heterocyclic group with the carbon atom and L^{C} to which they are attached, some —CH₂— in the heterocyclic group may be replaced by —O— or —S—, and L^{C} is —O— or —S—.

Specific examples of the acid labile group having the formula (AL-1) are shown below, but not limited thereto. * designates a point of attachment to the adjacent O—.

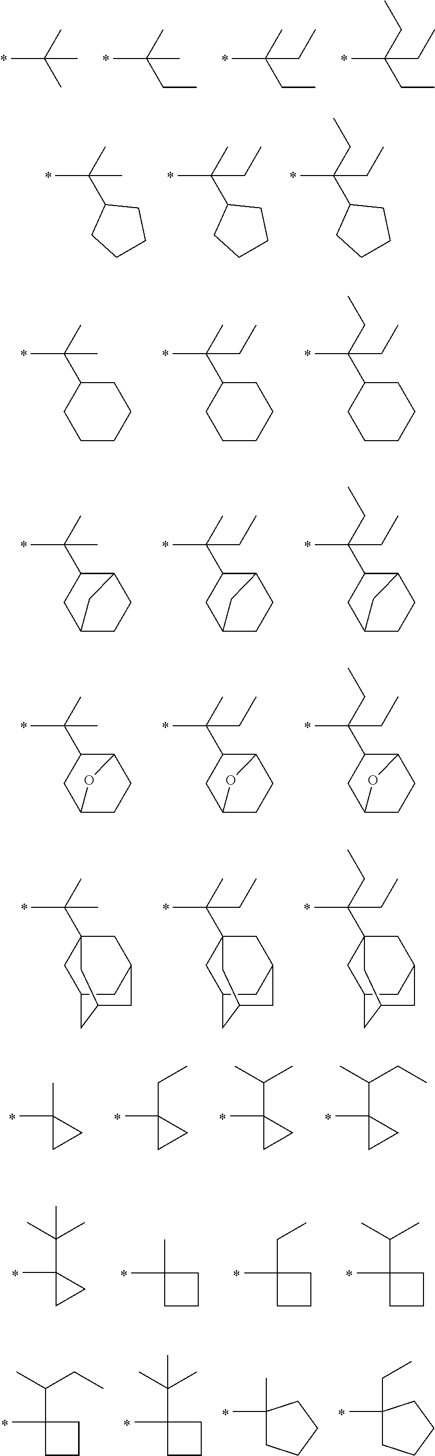

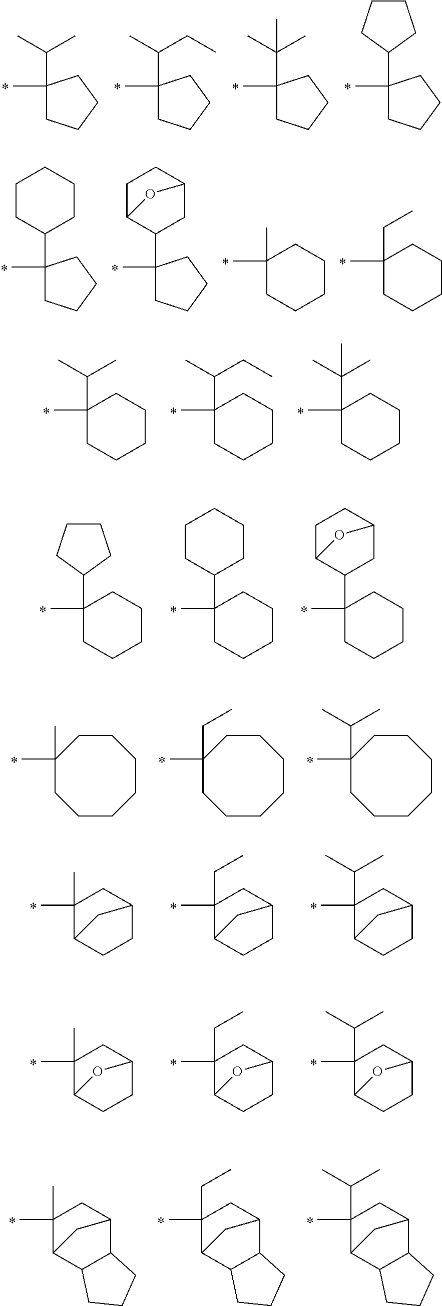

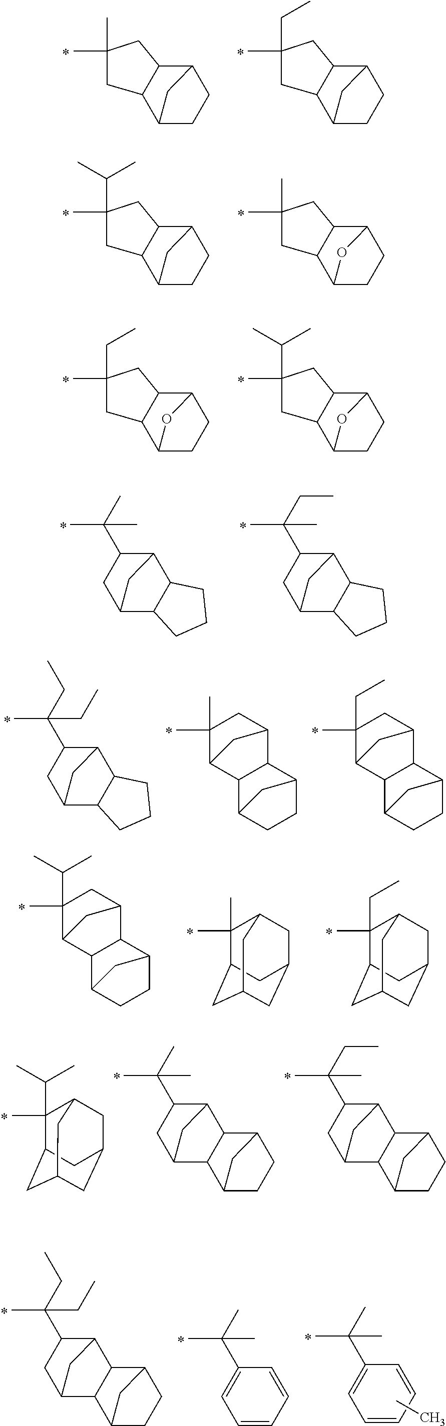

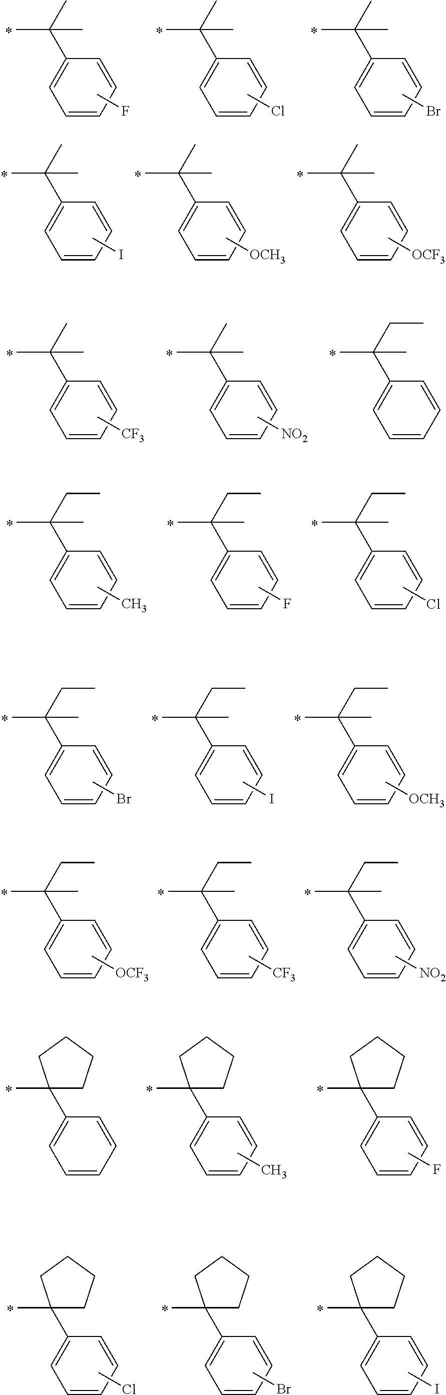

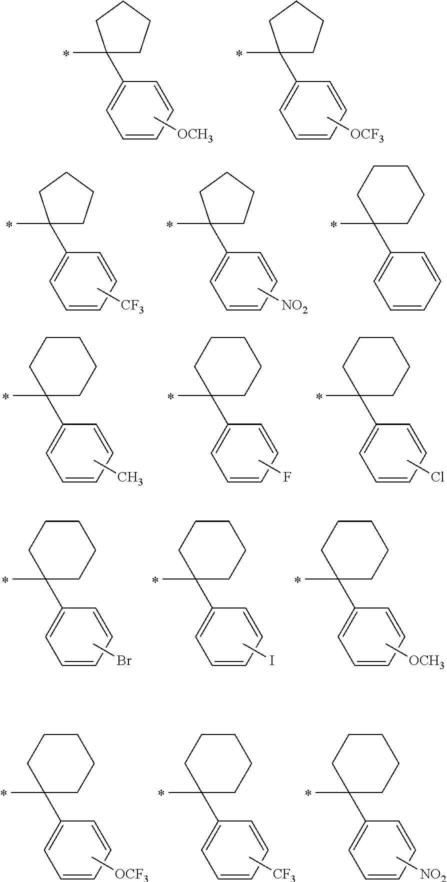

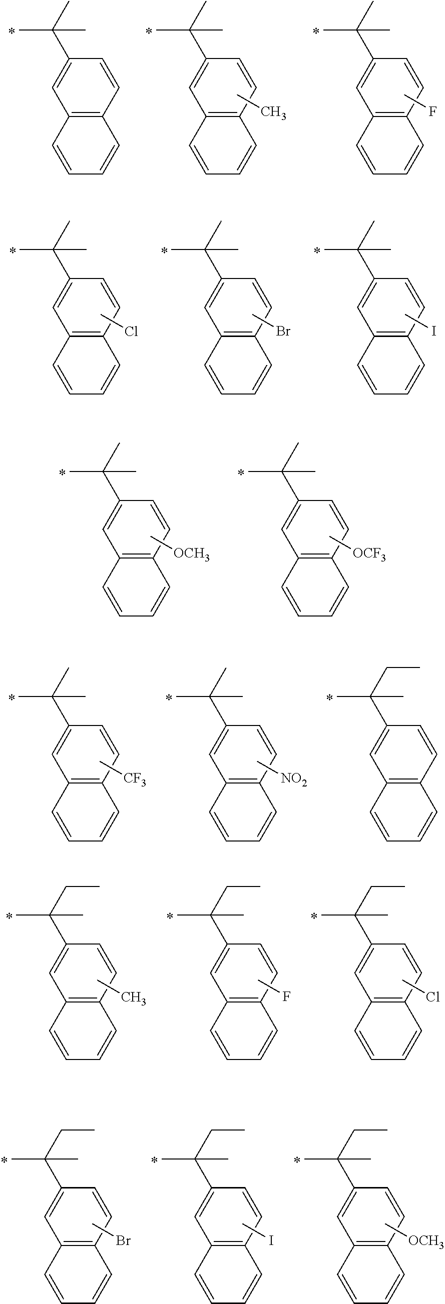

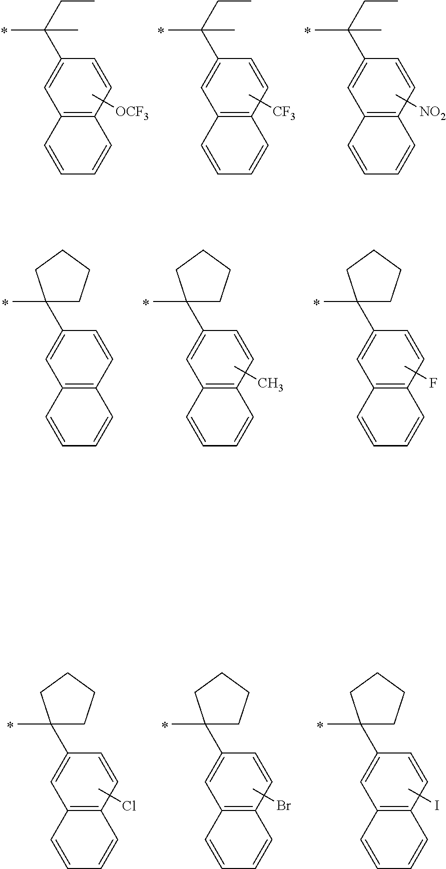

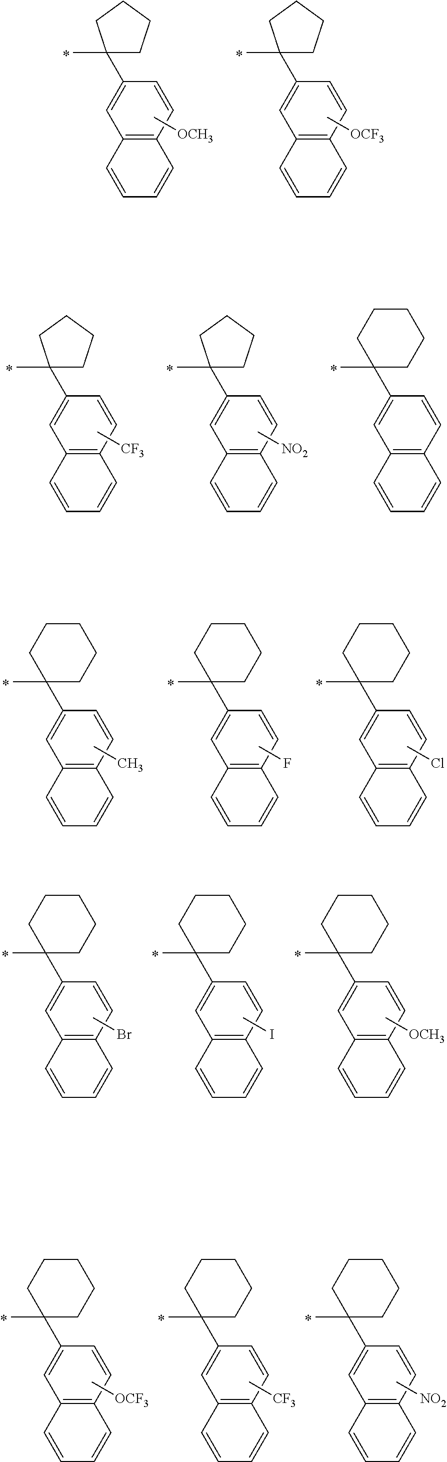

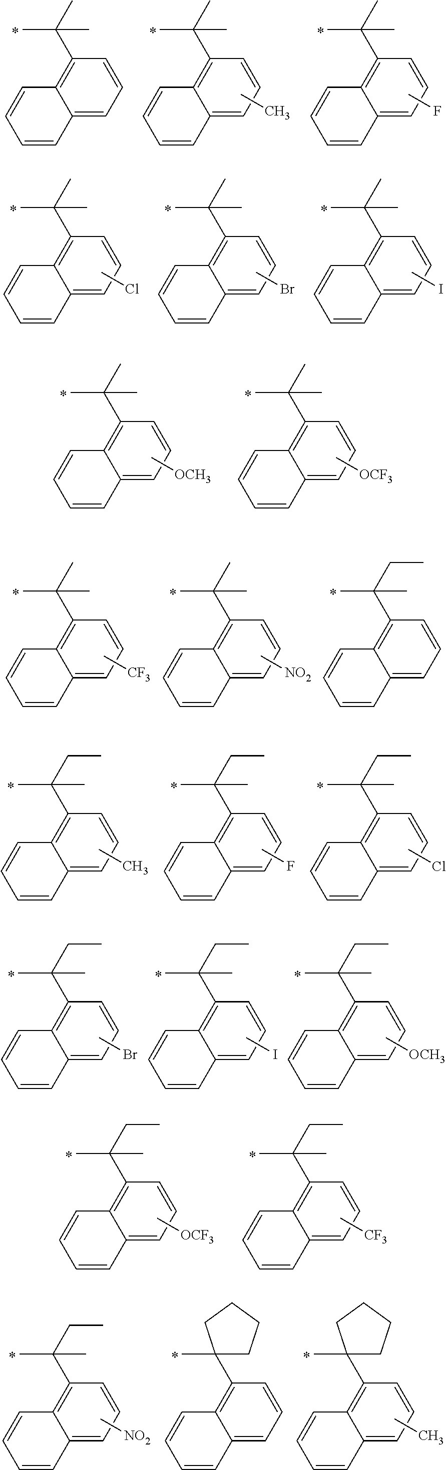

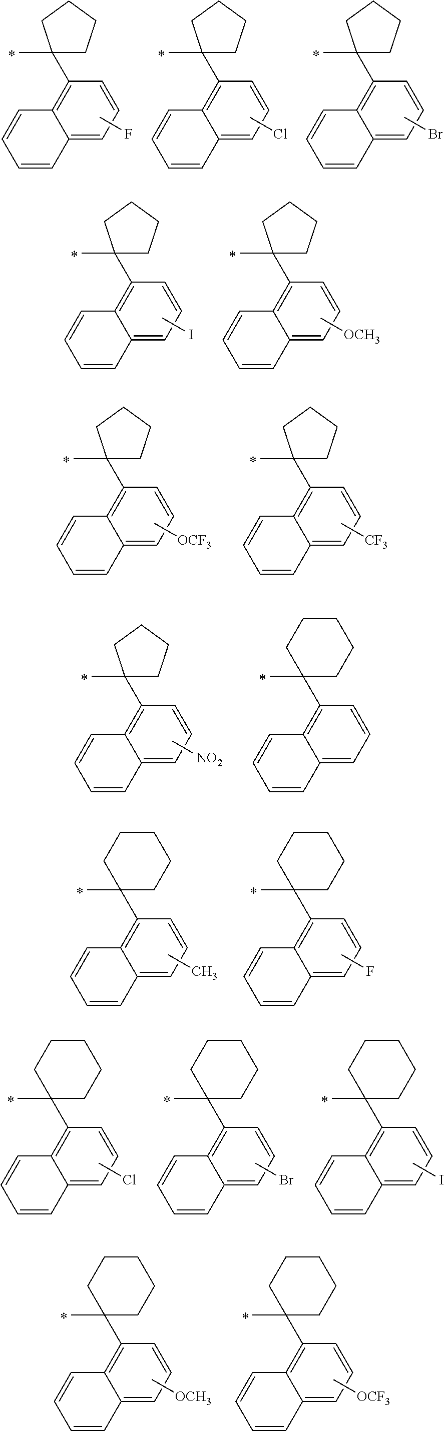

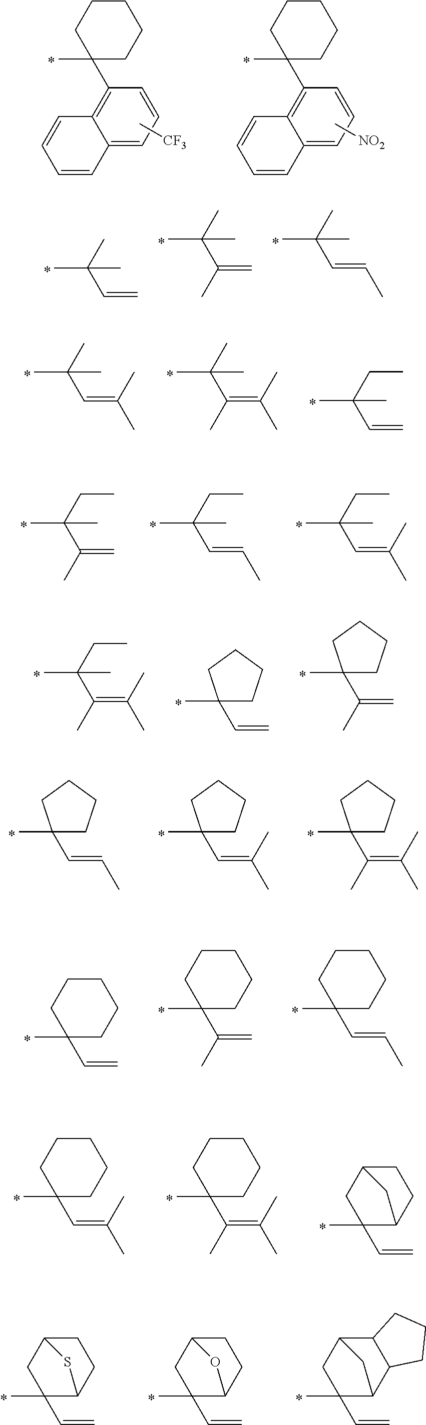

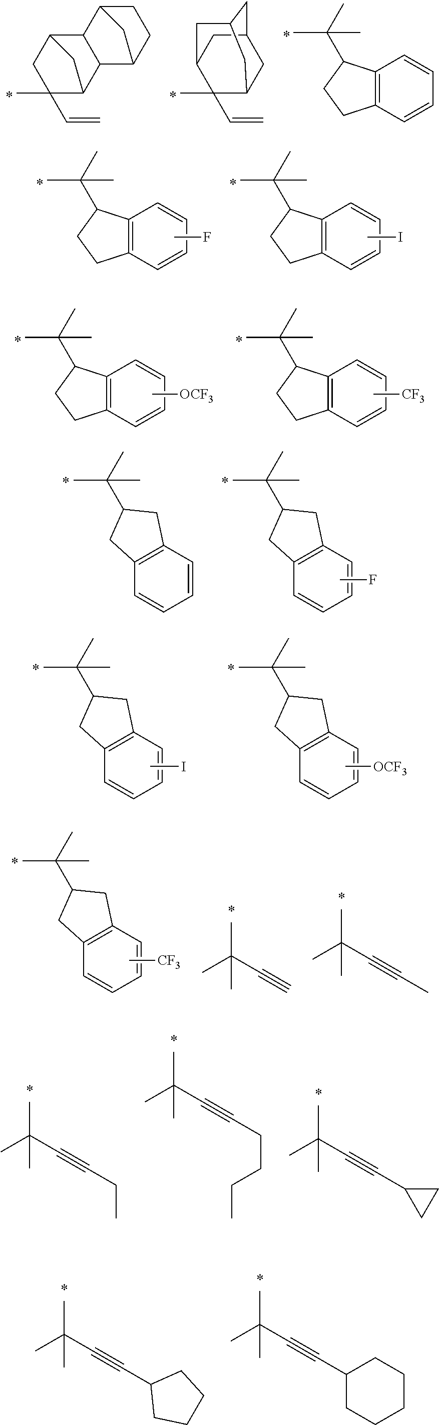

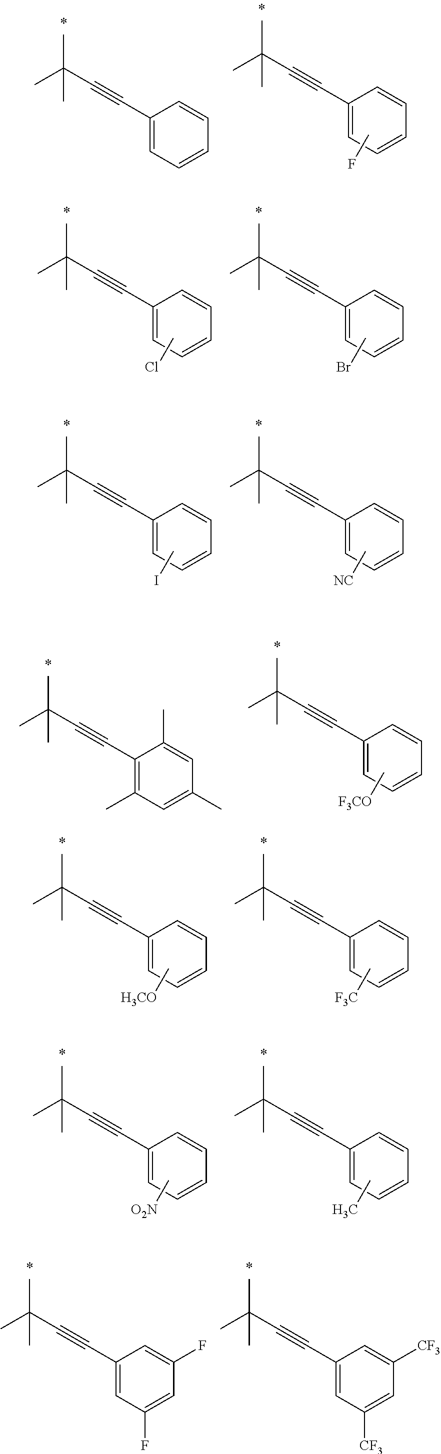

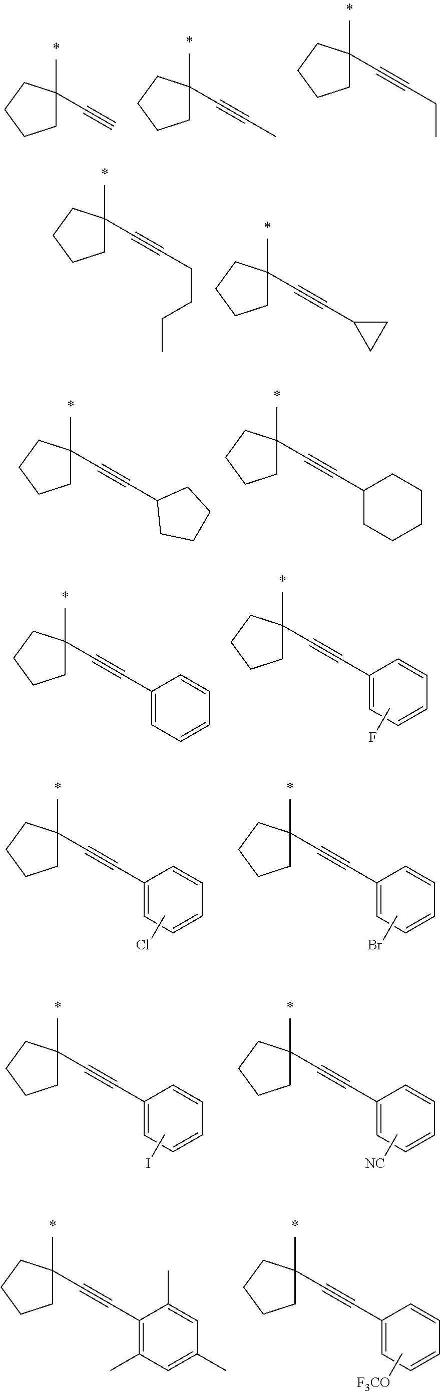

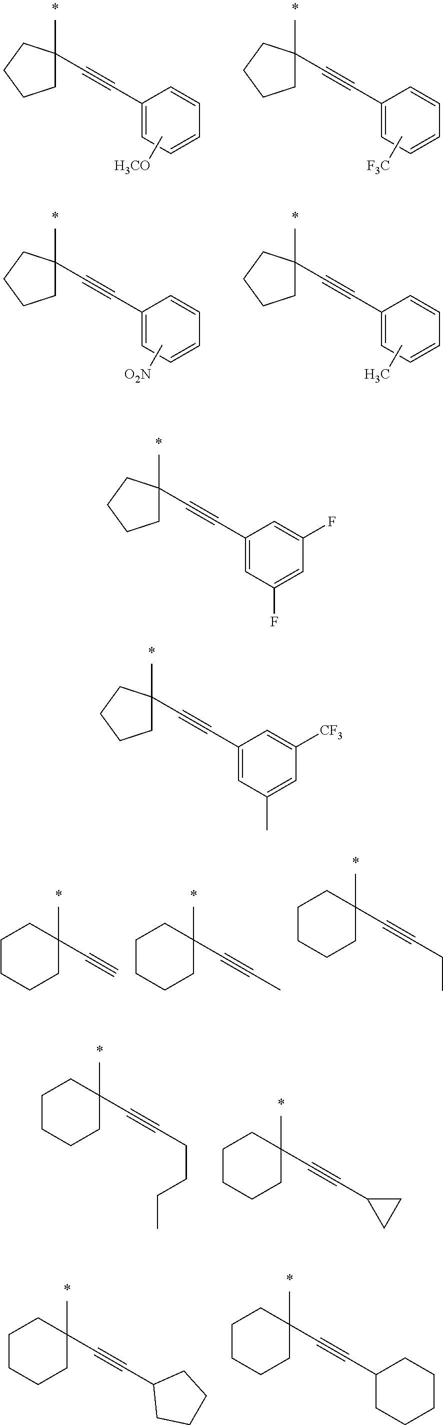

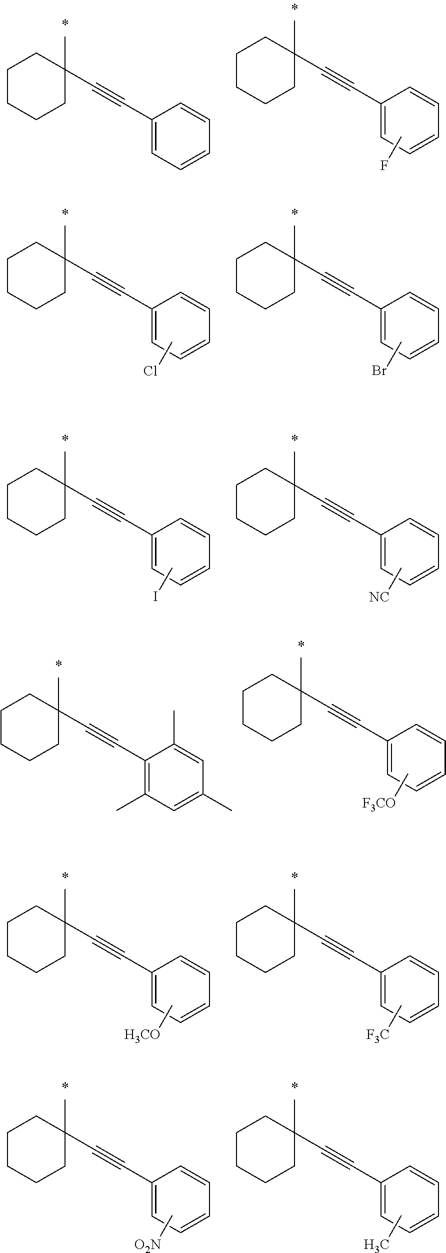

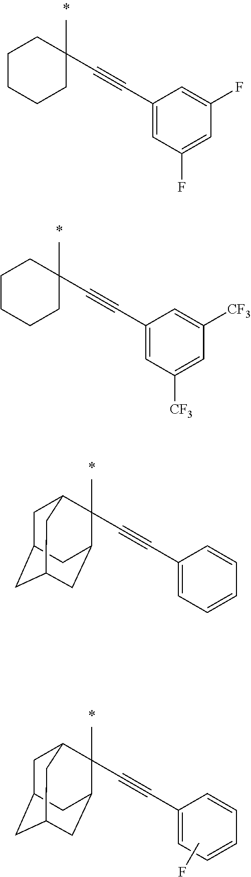

Specific examples of the acid labile group having the formula (AL-2) are shown below, but not limited thereto. * designates a point of attachment to the adjacent —O—.

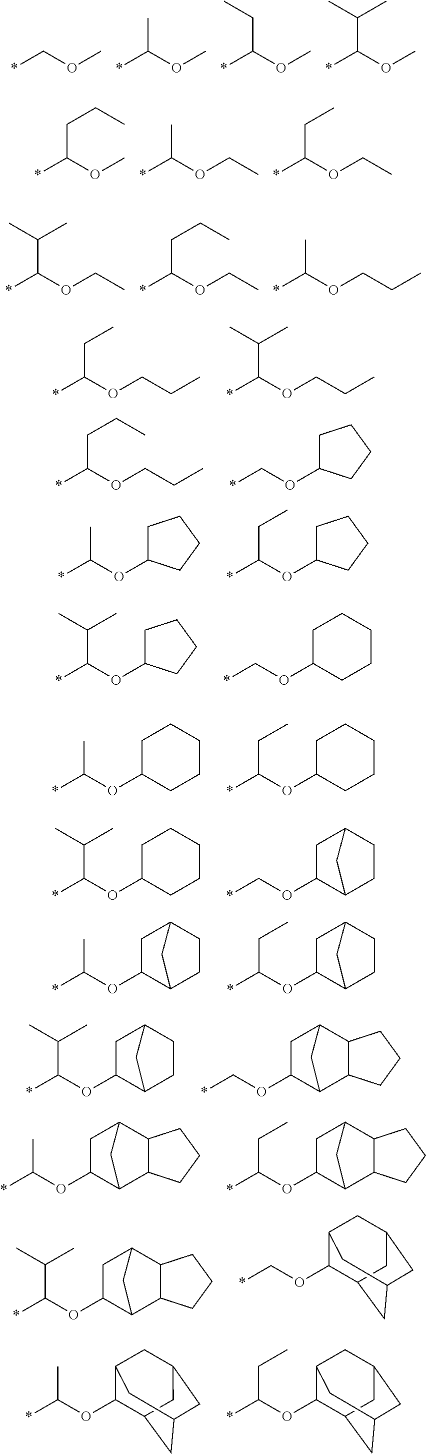

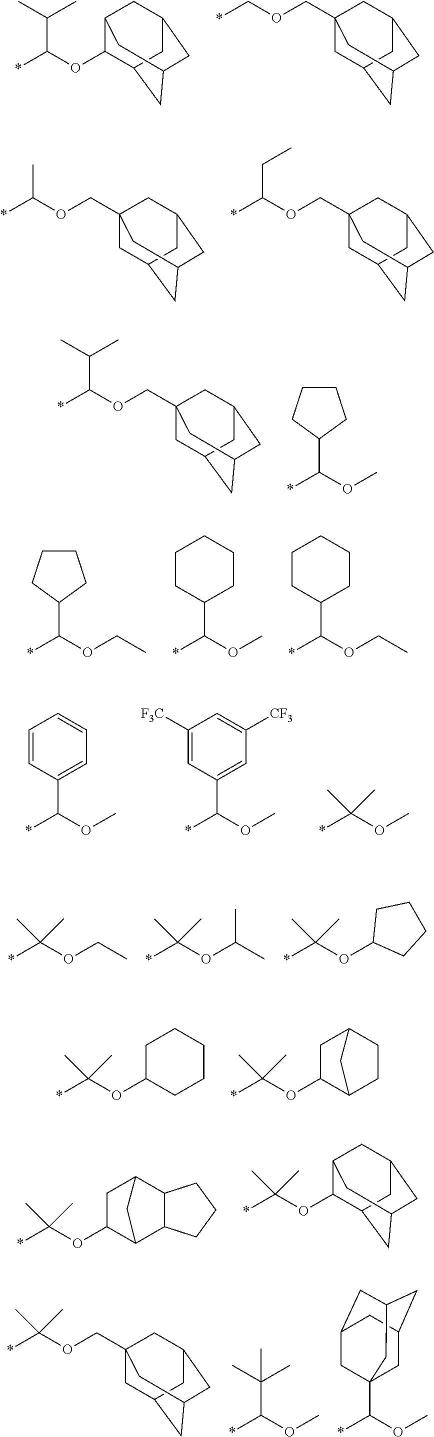

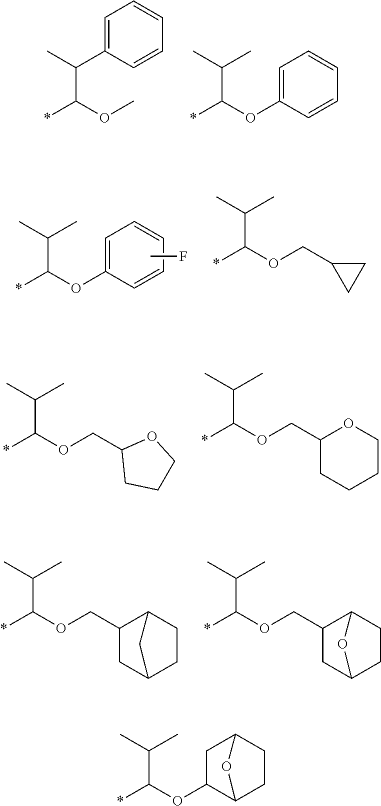

In the formula (1), at least one —SFs group and at least one —O—R^{AL} are attached to mutually adjacent carbon atoms. By being adjacent to each other, the acidity of the aromatic alcohol after —R^{AL} is deprotected is improved, and the dissolution contrast is improved.

In the formula (1), R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom include a fluorine atom, a chlorine atom, a bromine atom, and an iodine atom, and the halogen atom is preferably a fluorine atom or an iodine atom. The hydrocarbyl group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group and a naphthyl group; C₇-C₂₀ aralkyl groups such as a benzyl group, a 1-phenylethyl group, and a 2-phenylethyl group; and a group obtained by combining these groups. Among them, an aryl group is preferred. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. When n4 is 2, two R¹'s may be identical or different. Further, when n4 is 2, two R¹'s may bond together to form a ring with the carbon atom to which they are attached. The ring is preferably a 5- to 8-membered ring.

As the onium salt having the formula (1), an onium salt having the formula (1A) is preferred.

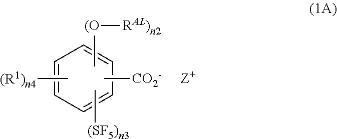

In the formula, n2 to n4, R¹, R^{AL}, and Z⁺ are as defined above.

Specific examples of the anion of the onium salt having the formula (1) are shown below, but not limited thereto. In the following formulae, Me is a methyl group. The substitution position of the substituent on the aromatic ring is not limited thereto as long as at least one —O—R^{AL} and at least one —SFs are disposed adjacent to each other.

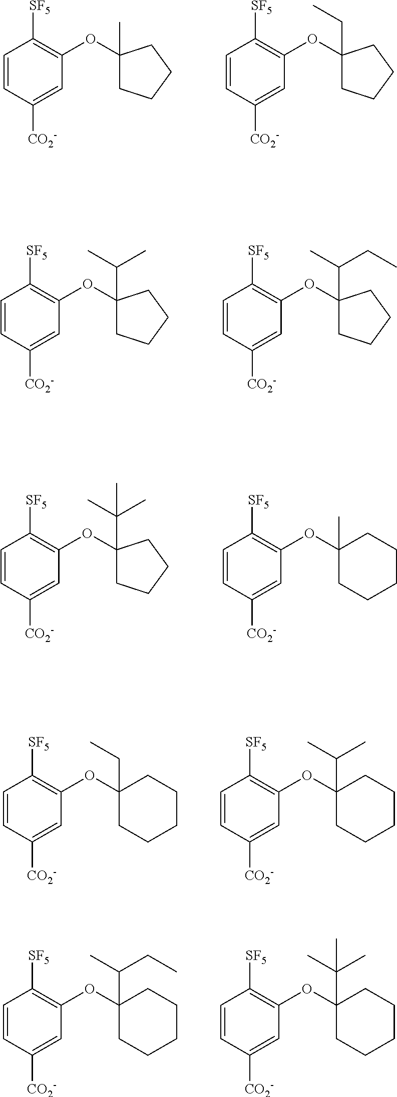

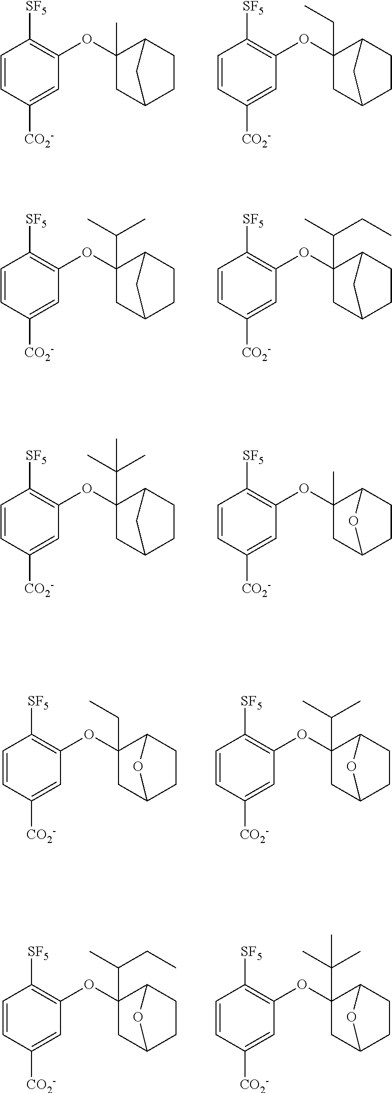

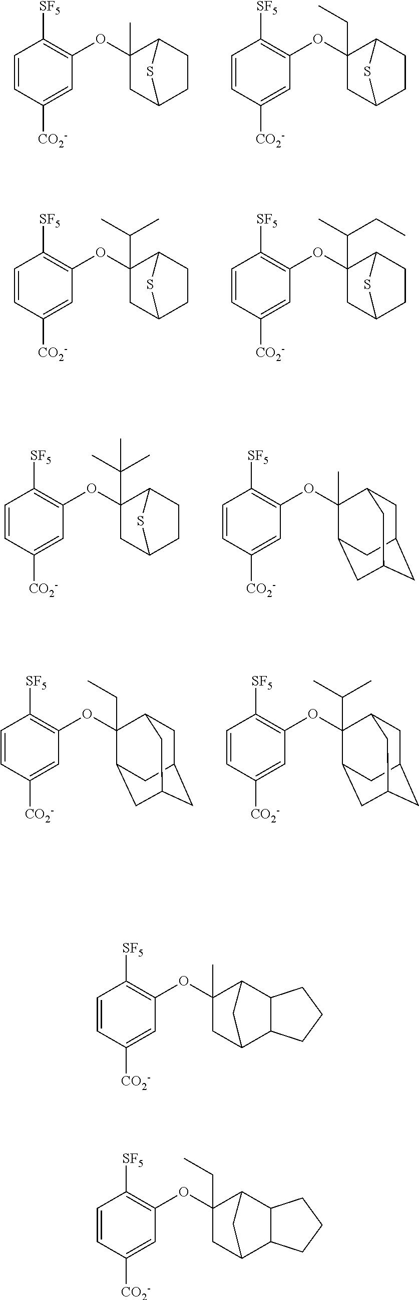

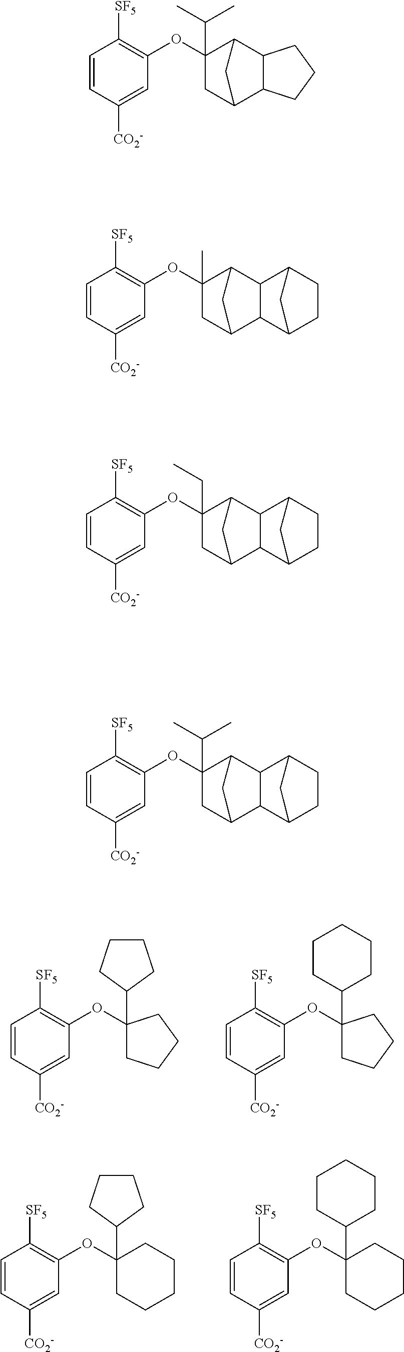

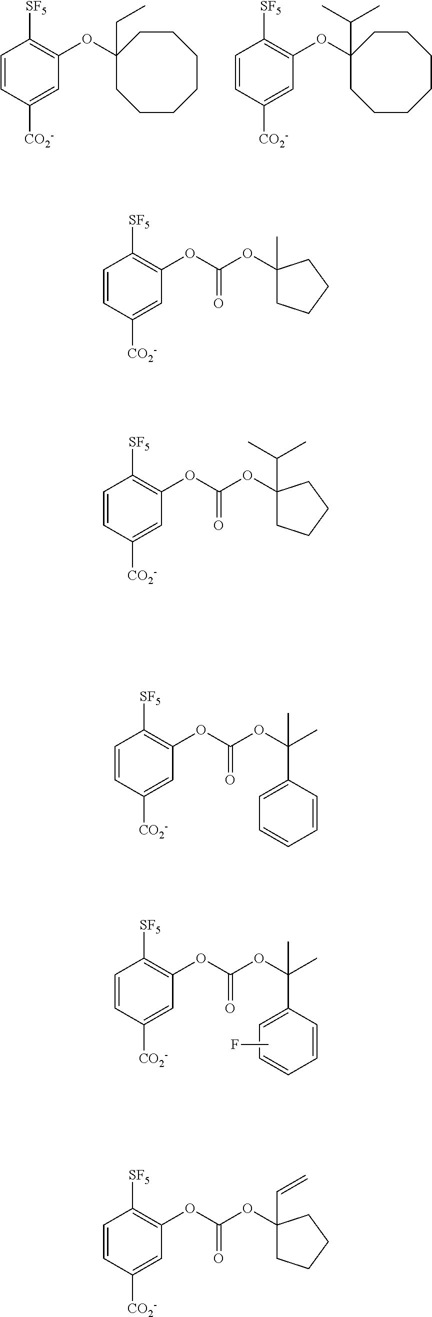

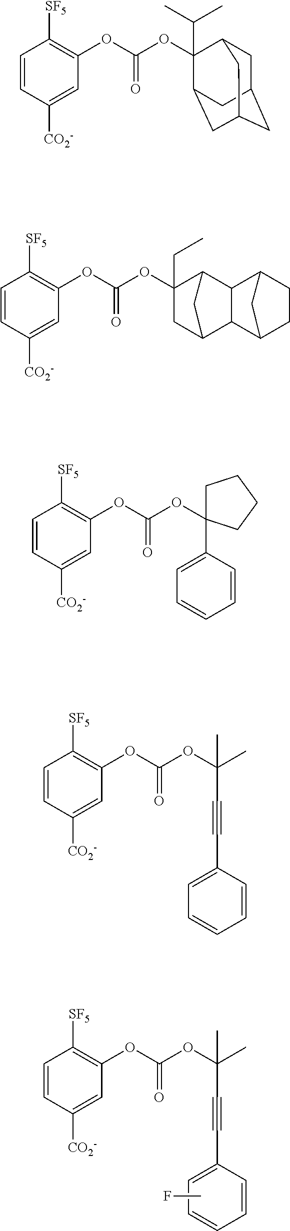

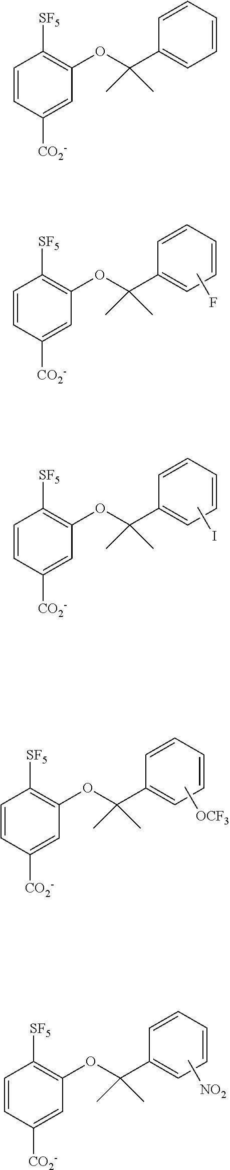

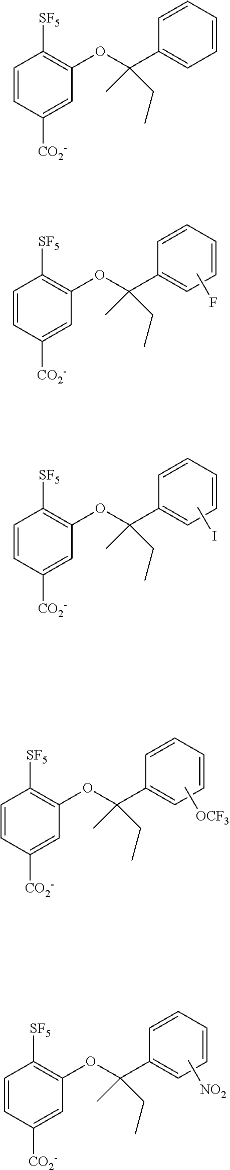

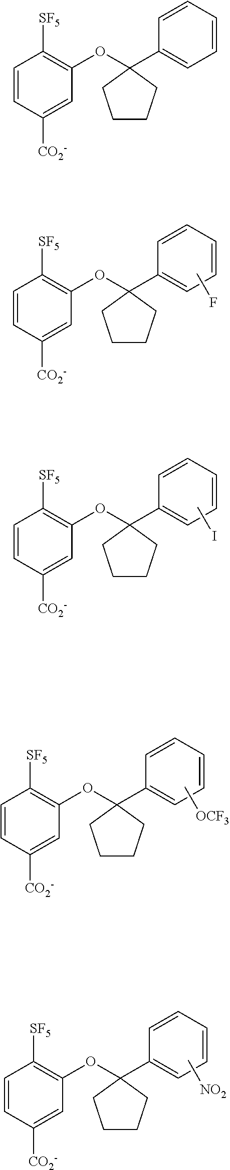

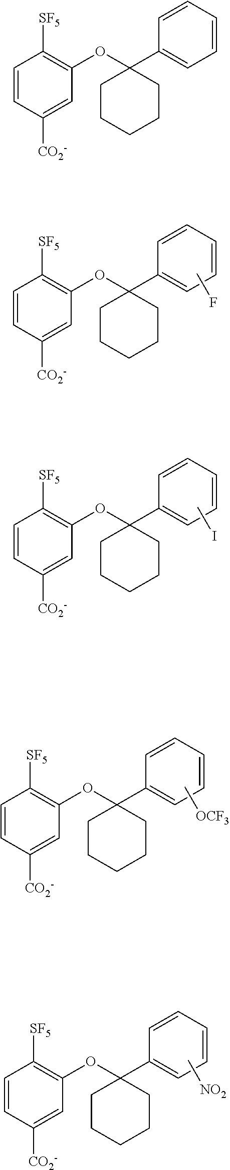

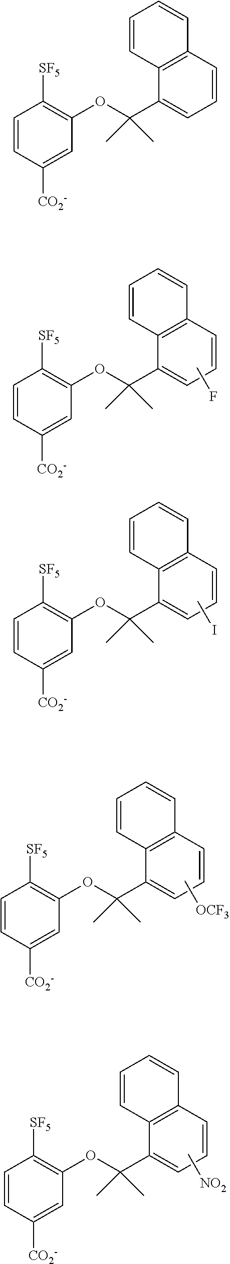

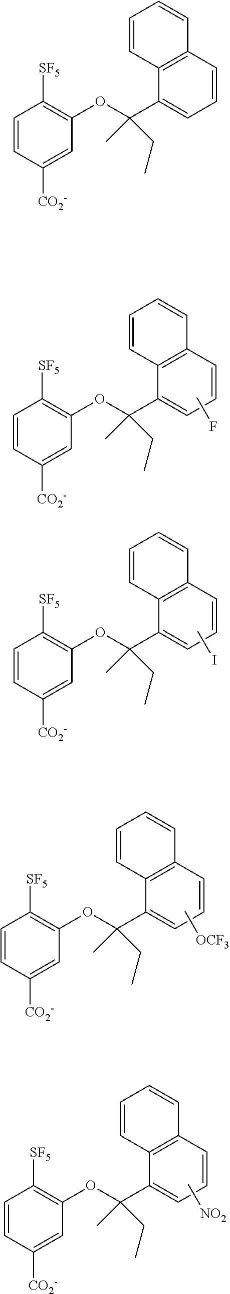

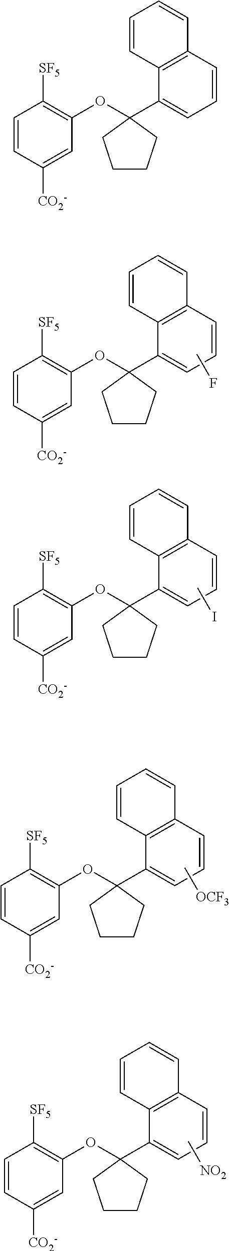

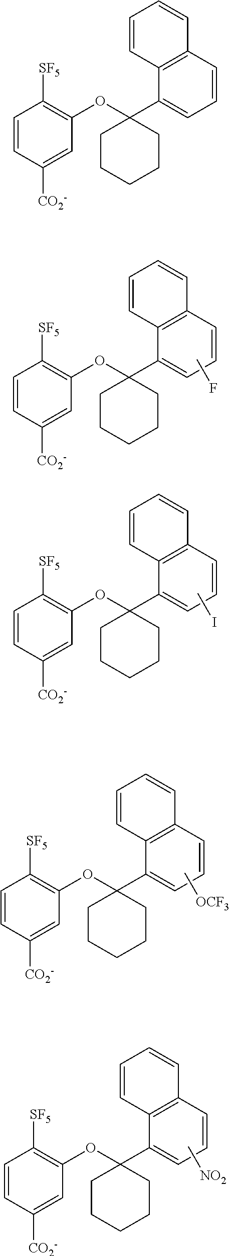

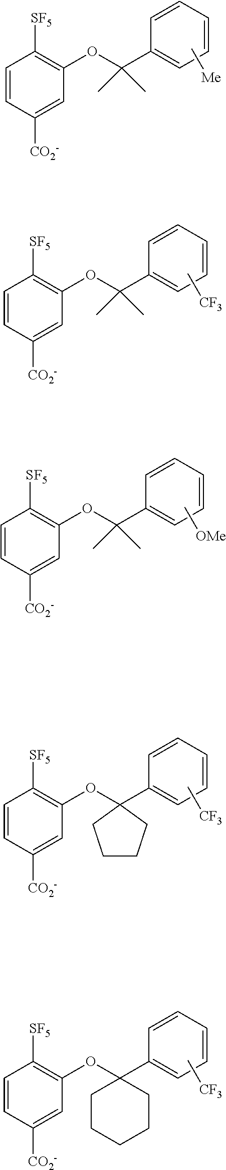

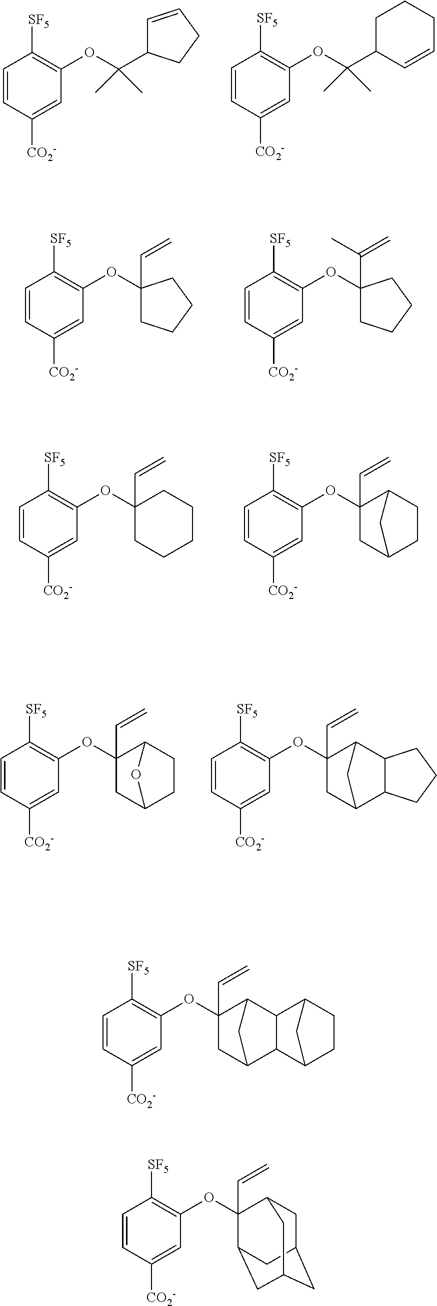

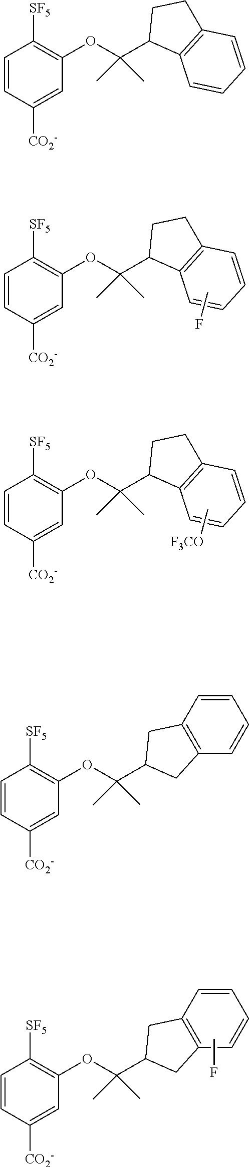

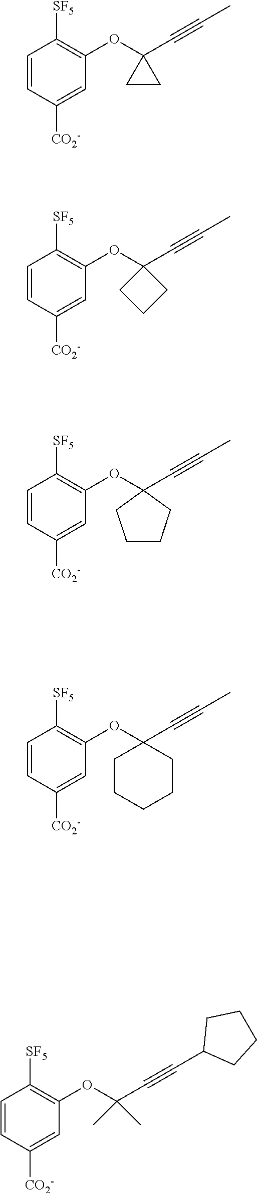

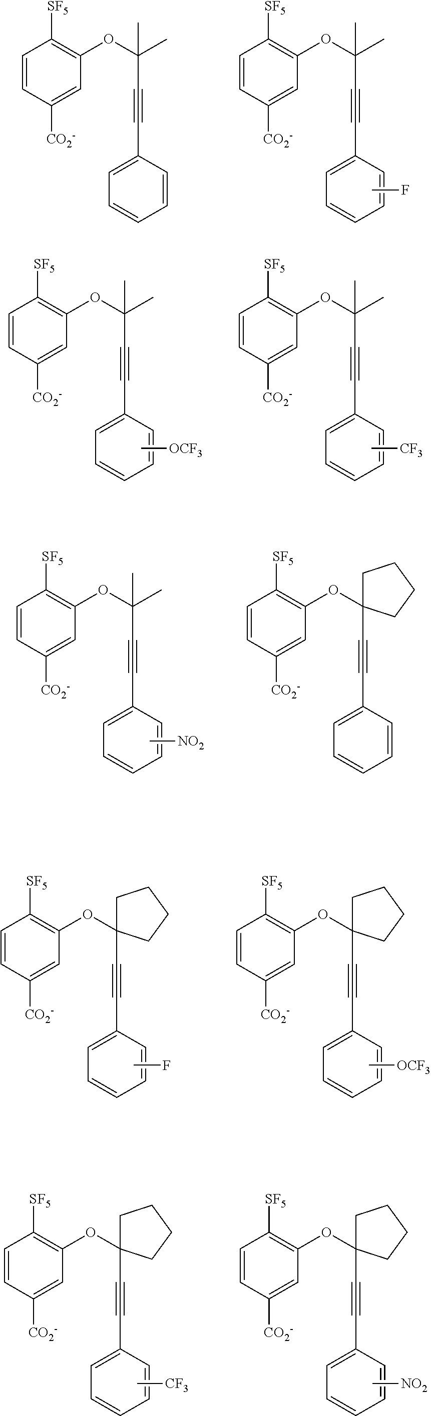

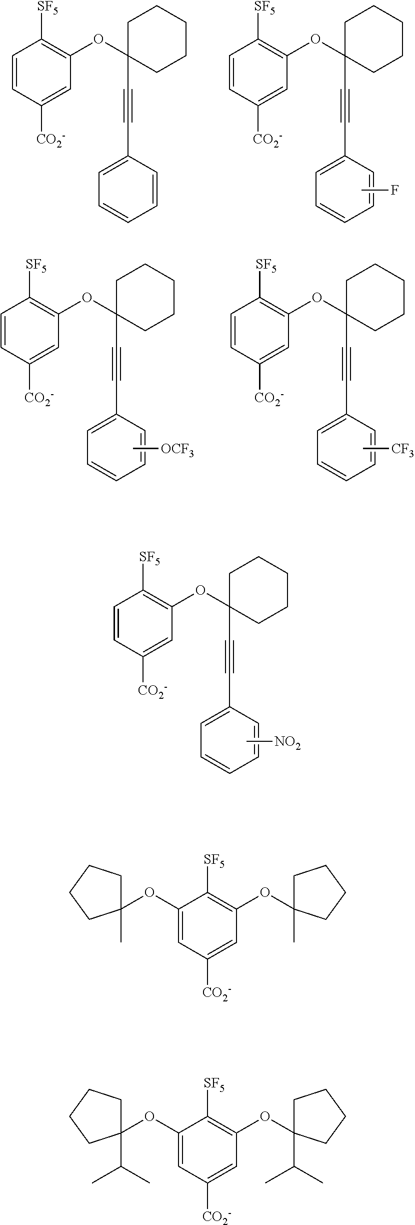

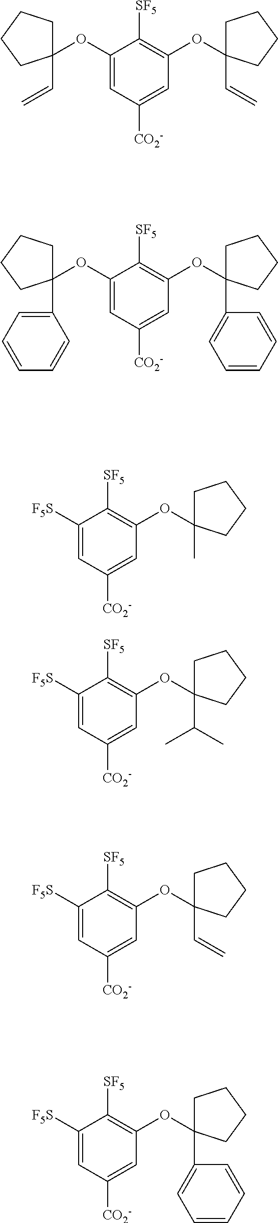

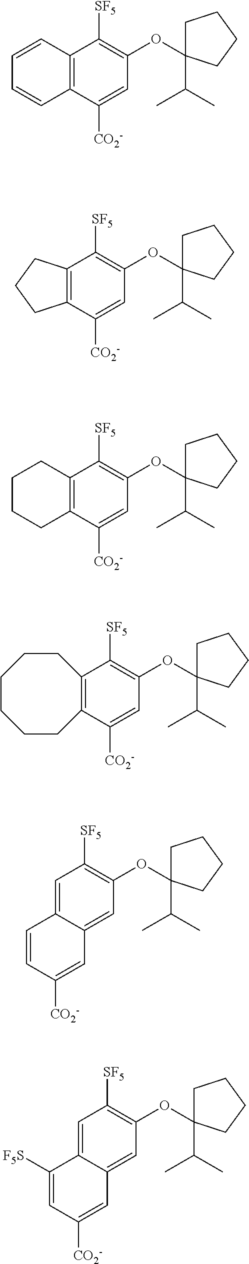

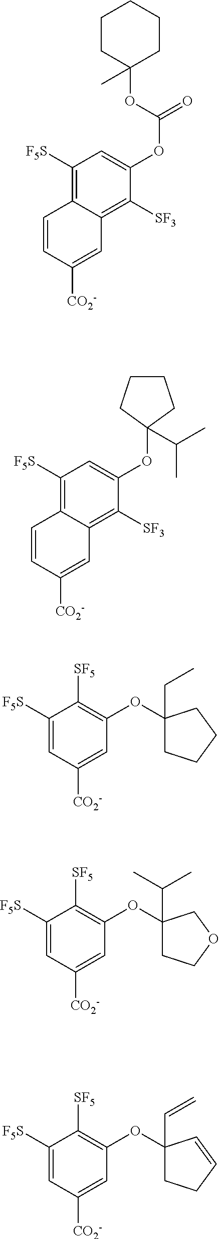

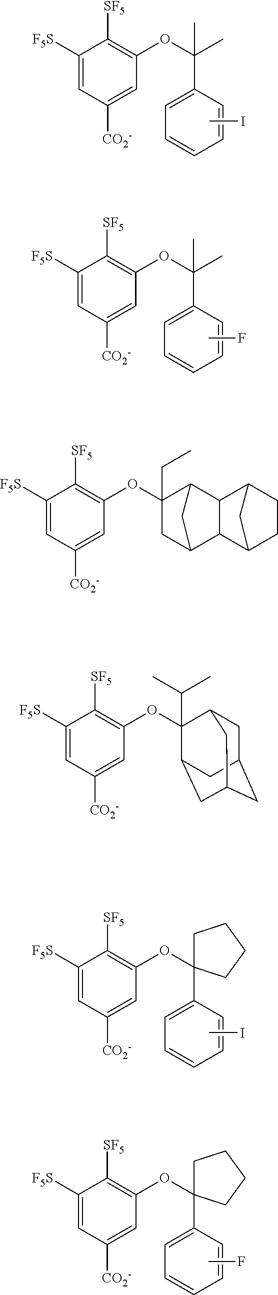

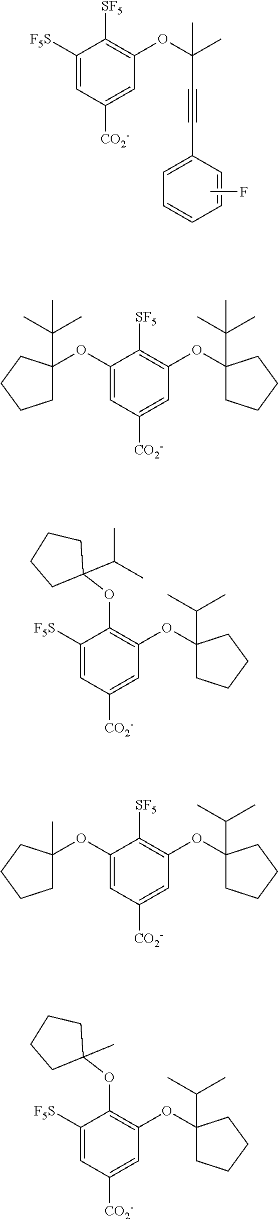

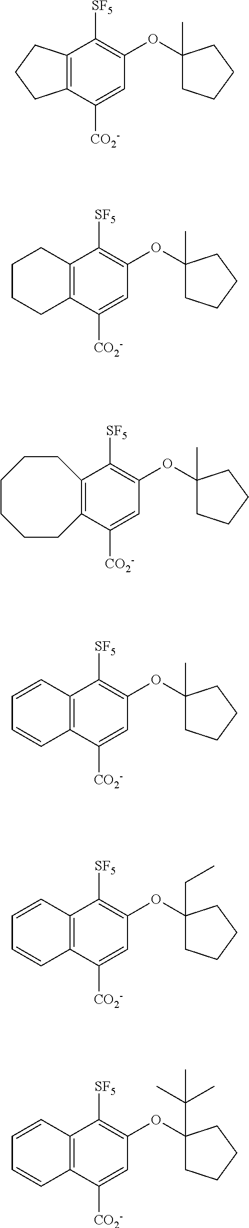

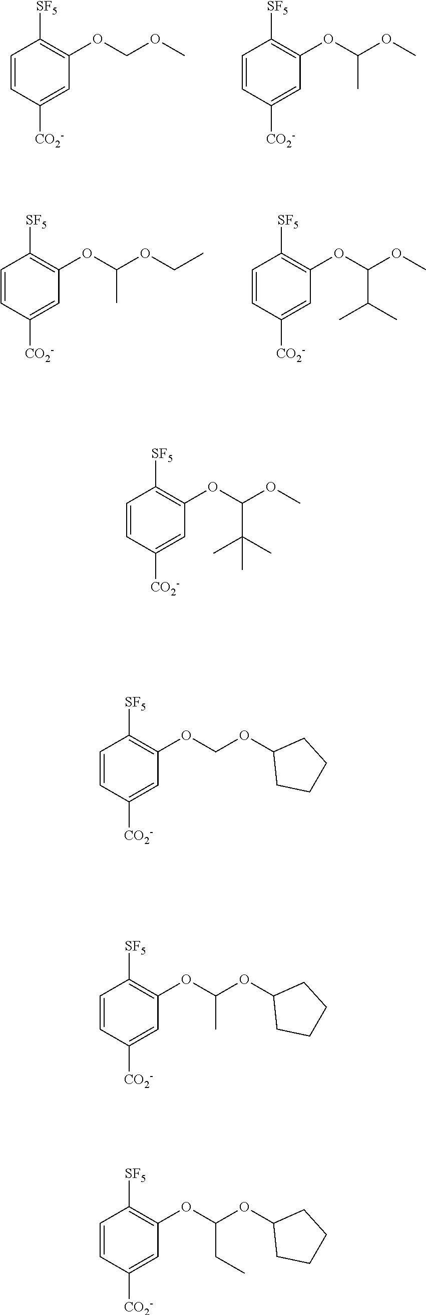

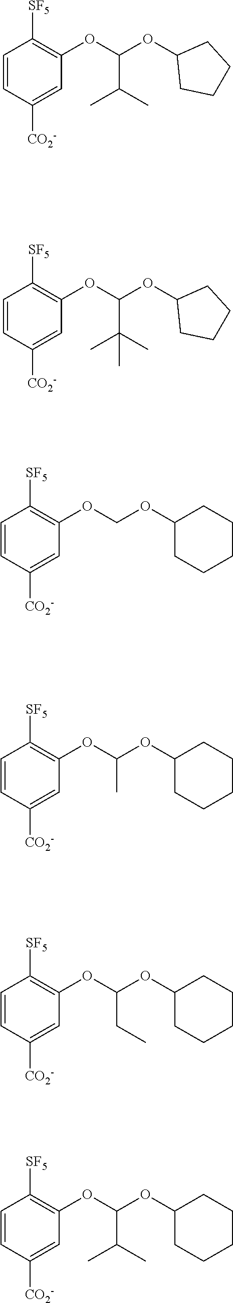

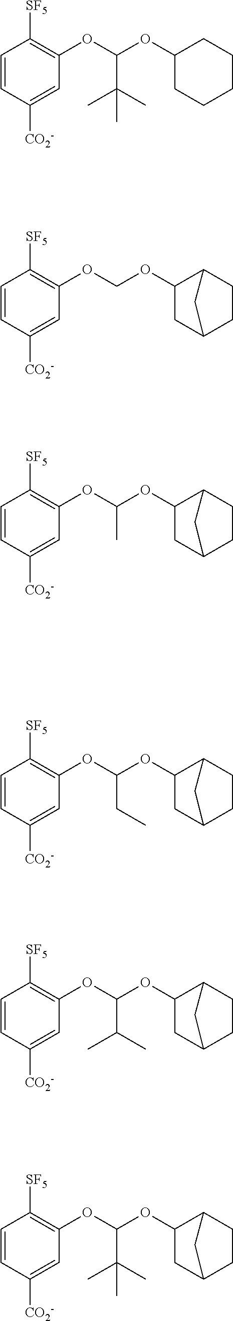

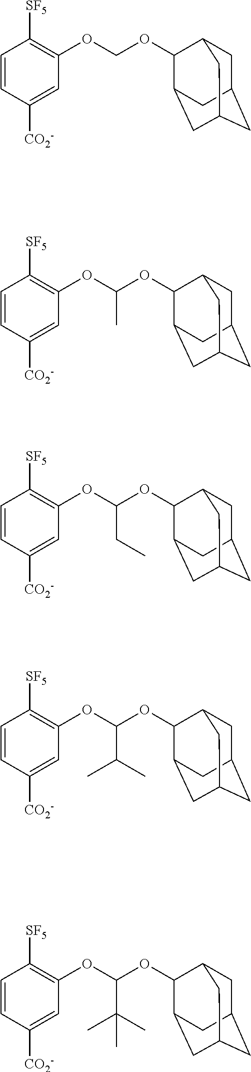

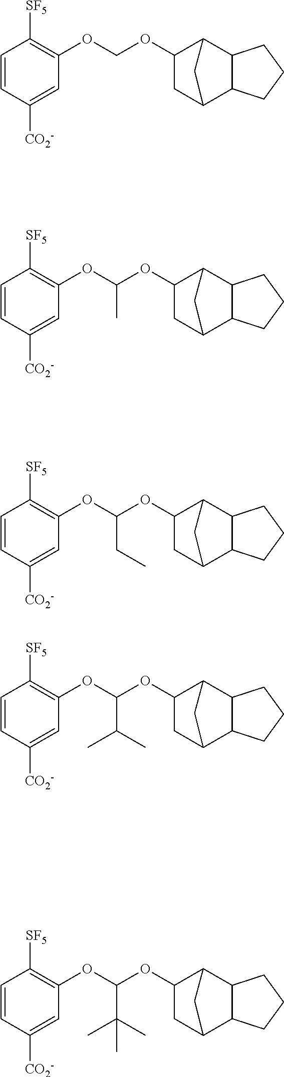

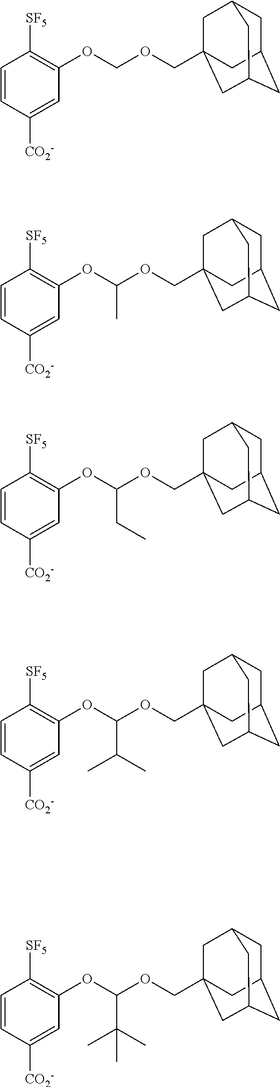

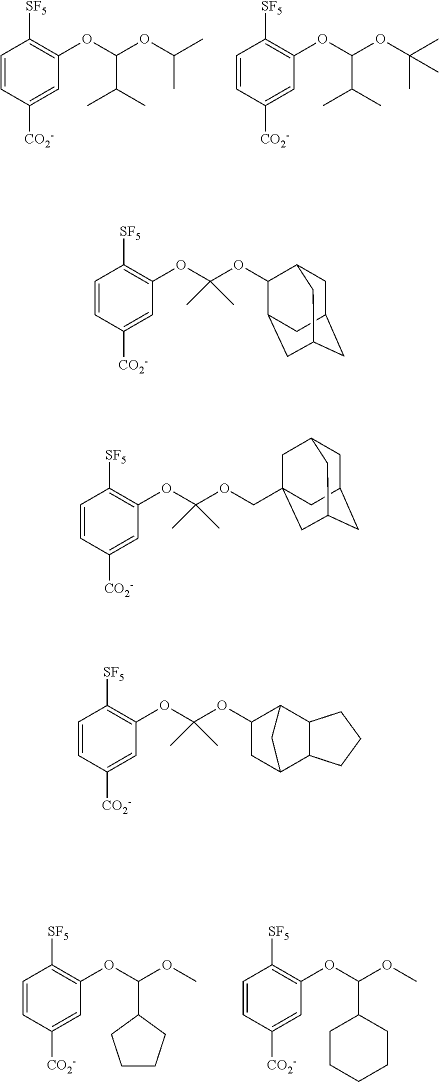

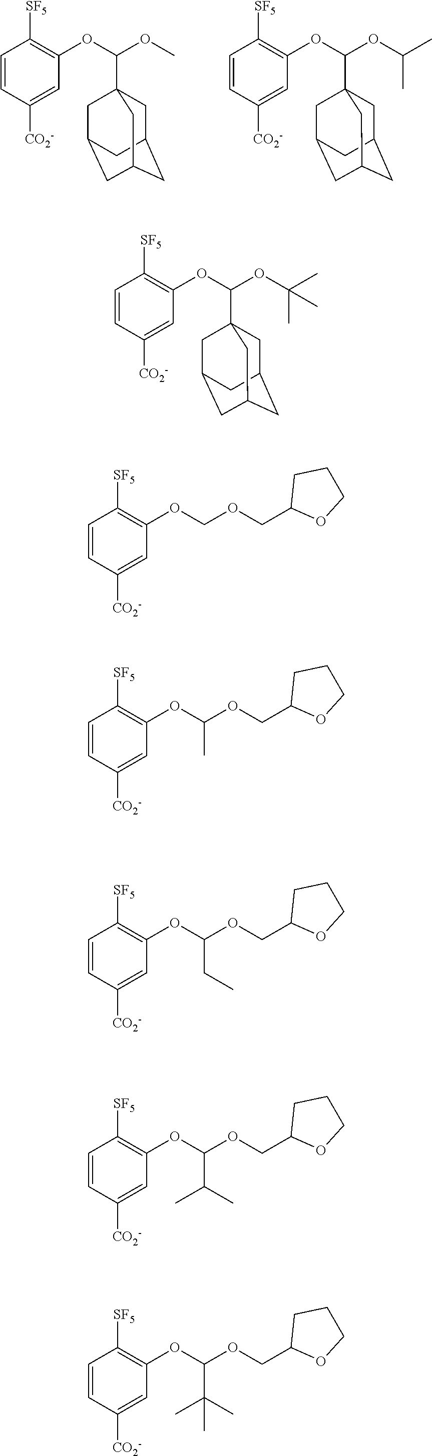

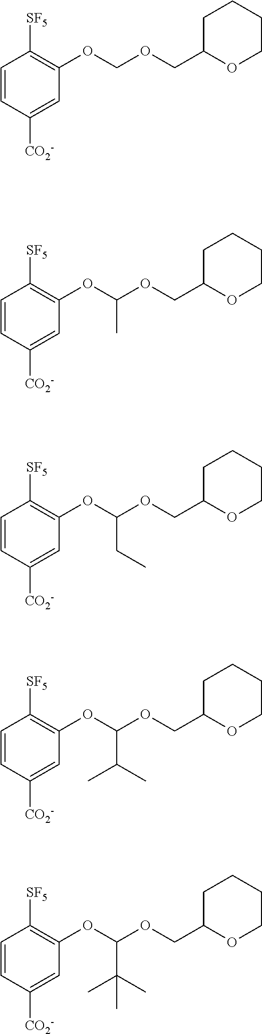

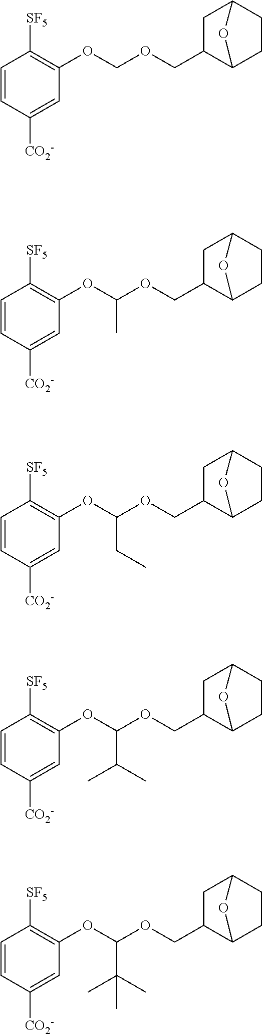

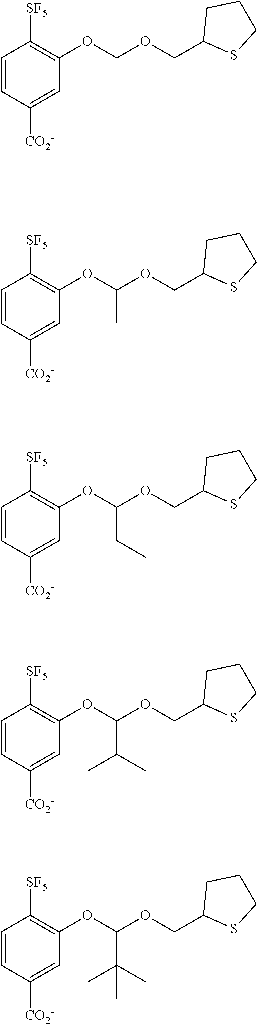

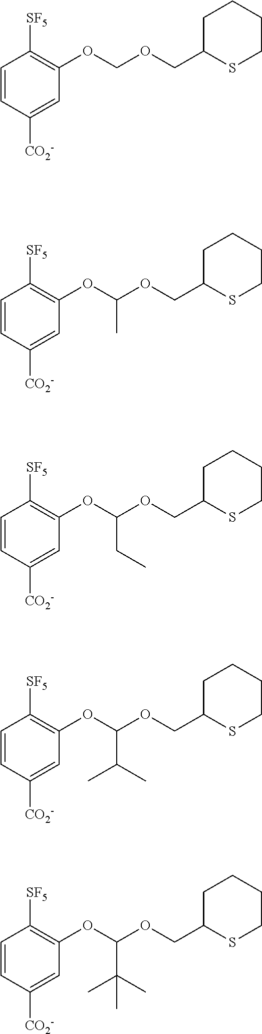

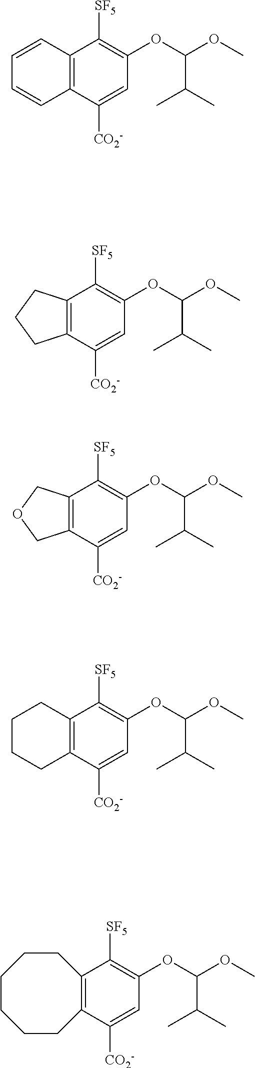

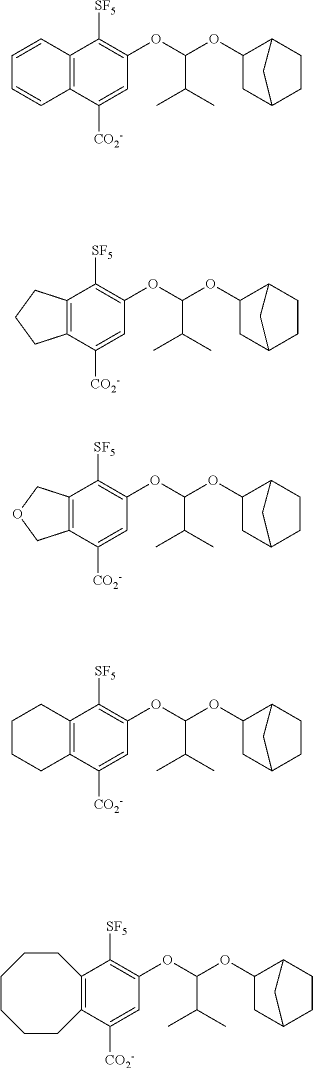

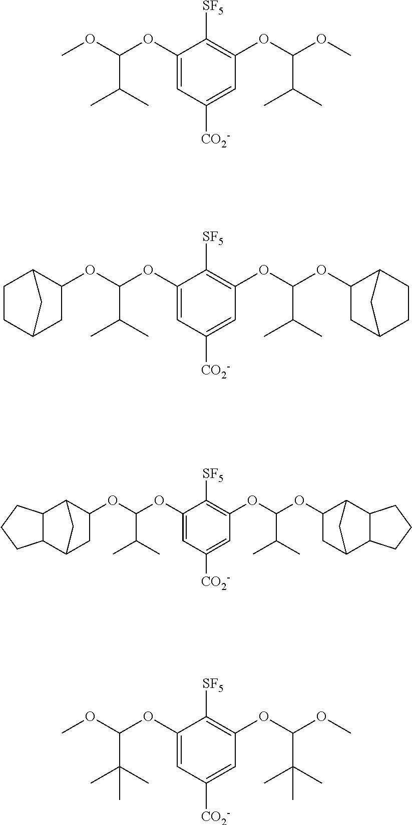

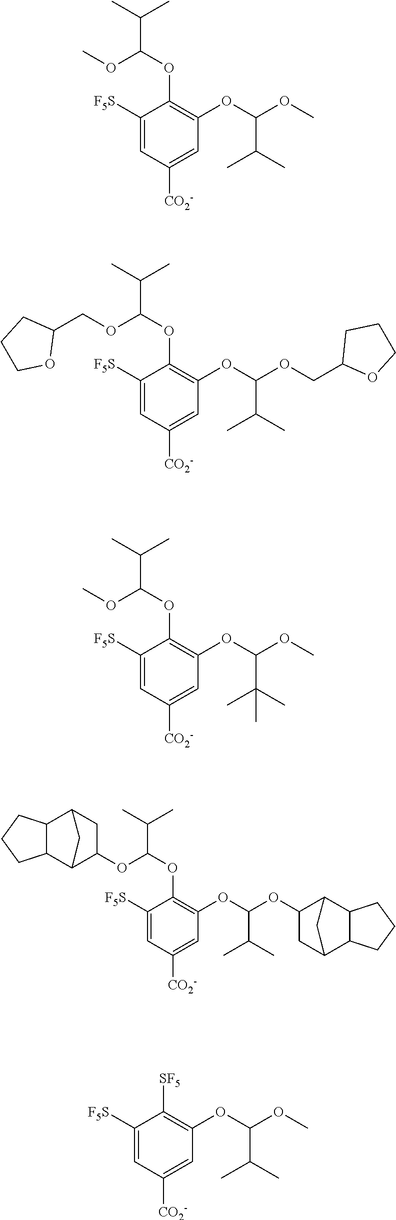

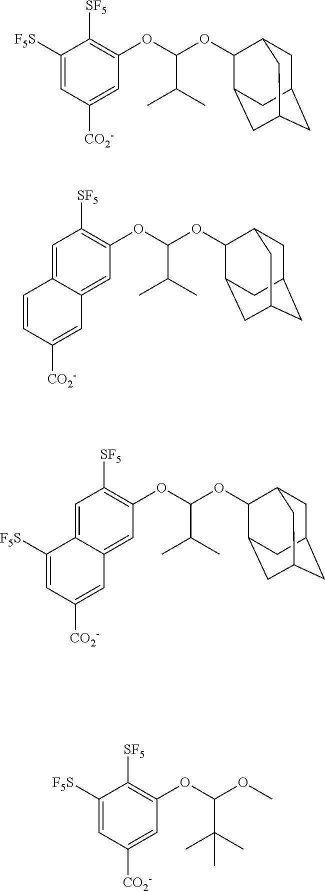

In the formula (1), Z⁺ is an onium cation. Examples of the onium cation include a sulfonium cation, an iodonium cation, an ammonium cation, and a phosphonium cation, and in particular, a sulfonium cation having the formula (Z-1), an iodonium cation having the formula (Z-2), or an ammonium cation having the formula (Z-3) is preferred.

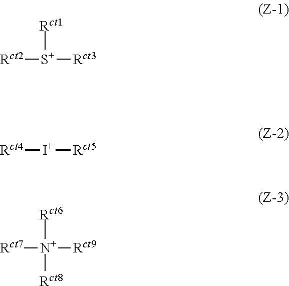

In the formulae (Z-1) to (Z-3), R^{ct1} to R^{ct9} are each independently a halogen atom or a C₁-C₃₀ hydrocarbyl group which may contain a heteroatom.

Specific examples of the halogen atom of Retl to R^{ct9} include a fluorine atom, a chlorine atom, a bromine atom, and an iodine atom.

The hydrocarbyl group of R^{ct1} to R^{ct9} may be saturated or unsaturated and straight, branched, or cyclic. Specific examples thereof include C₁-C₃₀ alkyl groups such as a methyl group, an ethyl group, a n-propyl group, an isopropyl group, a n-butyl group, an isobutyl group, a sec-butyl group, and a tert-butyl group; C₃-C₃₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₃₀ alkenyl groups such as a vinyl group, a 1-propenyl group, a 2-propenyl group, a butenyl group, and a hexenyl group; C₃-C₃₀ cyclic unsaturated hydrocarbyl groups such as a cyclohexenyl group; C₆-C₃₀ aryl groups such as a phenyl group, a naphthyl group, and a thienyl group; C₇-C₃₀ aralkyl groups such as a benzyl group, a 1-phenylethyl group, and a 2-phenylethyl group; and a group obtained by combining these groups, and aryl groups are preferred. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

R^{ct1} and R^{ct2} may bond together to form a ring with the sulfur atom to which they are attached. At this time, specific examples of the structure of the ring include those having the following formulae.

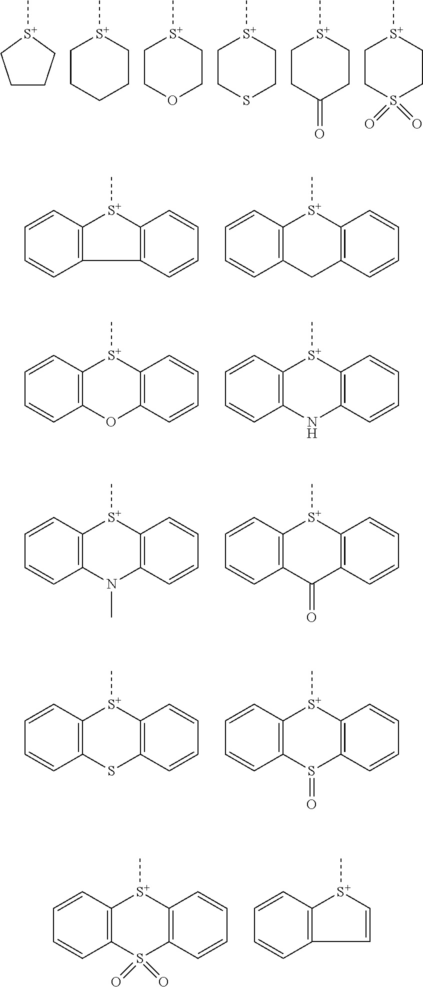

In the formulae, a broken line designates a point of attachment to R^{ct3}.

Further, any two of R^{ct6} to R^{ct9} may bond together to form a ring with the nitrogen atom to which they are attached.

Specific examples of the sulfonium cation having the formula (Z-1) include those described in JP-A 2024-003744, paragraphs to and those described in JP-A 2023-169812, paragraphs to [0085], but are not limited thereto.

Specific examples of the iodonium cation having the formula (Z-2) include those described in JP-A 2024-000259, paragraph [0181], but are not limited thereto.

Specific examples of the ammonium cation having the formula (Z-3) are shown below, but not limited thereto.

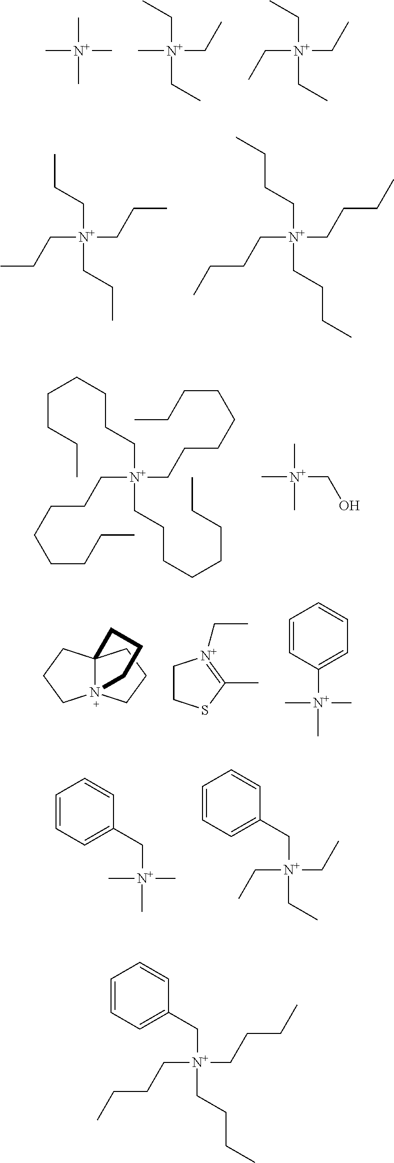

As the onium cation of Z⁺, a sulfonium cation having the formula (Z-4) is also preferred.

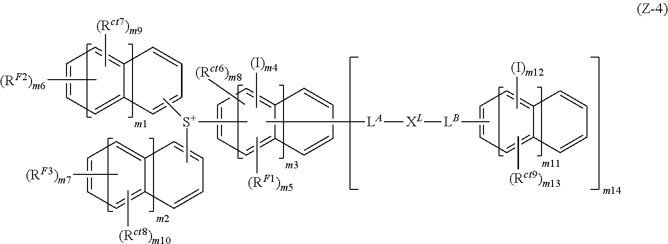

In the formula (Z-4), m1 is 0 or 1. The relevant structure is a benzene ring when m1 is 0 and a naphthalene ring when m1 is 1, and from the aspect of solvent solubility, a benzene ring when m1 is 0 is preferred. m2 is 0 or 1. The relevant structure is a benzene ring when m2 is 0 and a naphthalene ring when m2 is 1, and from the aspect of solvent solubility, a benzene ring when m2 is 0 is preferred. m3 is 0 or 1. The relevant structure is a benzene ring when m3 is 0 and a naphthalene ring when m3 is 1, and from the aspect of solvent solubility, a benzene ring when m3 is 0 is preferred.

In the formula (Z-4), m4 is 0, 1, 2, 3, or 4. The larger the number of iodine atoms in the cationic structure is, the more the absorption to EUV is particularly enhanced, but since there is a concern that the solvent solubility is reduced and precipitation occurs in the resist composition, and therefore m4 is preferably 0, 1, 2, or 3, and more preferably 0, 1, or 2.

In the formula (Z-4), m5 is 0, 1, 2, 3, or 4. From the aspect of raw material procurement, m5 is preferably 0, 1, 2, or 3, and more preferably 0, 1, or 2. m6 is 0, 1, 2, 3, 4, 5, or 6. From the aspect of raw material procurement, m6 is preferably 0, 1, 2, or 3, and more preferably 0, 1, or 2. m7 is 0, 1, 2, 3, 4, 5, or 6. From the aspect of raw material procurement, m7 is preferably 0, 1, 2, or 3, and more preferably 0, 1, or 2.

In the formula (Z-4), m8 is 0, 1, or 2. From the aspect of raw material procurement, m8 is preferably 0 or 1. m9 is 0, 1, or 2. From the aspect of raw material procurement, m9 is preferably 0 or 1. m10 is 0, 1, or 2. From the aspect of raw material procurement, m10 is preferably 0 or 1.

In the formula (Z-4), m11 is 0 or 1. The relevant structure is a benzene ring when m11 is 0 and a naphthalene ring when m11 is 1, and from the aspect of solvent solubility, a benzene ring when m11 is 0 is preferred.

In the formula (Z-3), m12 is 0, 1, 2, 3, or 4. The larger the number of iodine atoms in the cationic structure is, the more the absorption to EUV is particularly enhanced, but since there is a concern that the solvent solubility is reduced and precipitation occurs in the resist composition, and therefore m12 is preferably 0, 1, 2, or 3, and more preferably 0, 1, or 2.

In the formula (Z-4), m13 is 0, 1, or 2. From the aspect of raw material procurement, m13 is preferably 0 or 1. m14 is 0, 1, or 2. From the aspect of synthesis, m14 is preferably 0 or 1.

These subscripts meet 0≤m6+m9≤4 when m1 is 0, 0≤m6+m9≤6 when m1 is 1, 0≤m7+m10≤4 when m2 is 0, 0≤m7+m10≤6 when m2 is 1, 1≤m4+m5+m8+m14≤4 when m3 is 0, 1≤m4+m5+m8+m14≤6 when m3 is 1, 0≤m12+m13≤4 when m11 is 0, and 0≤m12+m13≤6 when m11 is 1, and m4+m12≥1.

In the formula (Z-4), RFI to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Among them, a trifluoromethyl group, a trifluoromethoxy group, and a trifluorothiomethoxy group are preferred. When m5 is 2 or more, a plurality of RFI's may be identical or different, when m6 is 2 or more, a plurality of R^{F2}'s may be identical or different, when m7 is 2 or more, a plurality of R^{F3}'s may be identical or different.

In the formula (Z-4), R^{ct6} to R^{ct9} are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and the hydrocarbyl moiety of the hydrocarbyloxy group and the hydrocarbylthio group may be saturated or unsaturated and straight, branched, or cyclic. Specific examples thereof are as exemplified above for the hydrocarbyl group of R¹ in the description of the formula (1). Some or all hydrogen atoms in the hydrocarbyl group and the hydrocarbyl moiety of the hydrocarbyloxy group and the hydrocarbylthio group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Further, when m8 is 2, two R^{ct6}'s may be identical or different and two R^{ct6}'s may bond together to form a ring with the carbon atom to which they are attached, when m9 is 2, two R^{ct7}'s may be identical or different and two R^{ct7}'s may bond together to form a ring with the carbon atom to which they are attached, when m10 is 2, two R^{ct8}'s may be identical or different and two R^{ct8}'s may bond together to form a ring with the carbon atom to which they are attached, when m13 is 2, two R^{ct9}'s may be identical or different and two R^{ct9}'s may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. Some or all hydrogen atoms in the ring may be substituted with a group containing a heteroatom such as an oxygen atom, a sulfur atom, a nitrogen atom, or a halogen atom, and some —CH₂— in the ring may be replaced by a group containing a heteroatom such as an oxygen atom, a sulfur atom, or a nitrogen atom, so that the ring may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

The aromatic rings directly attached to S⁺ in the sulfonium cation having the formula (Z-4) may bond together to form a ring with S⁺. At this time, specific examples of the structure of the ring include those having the following formulae.

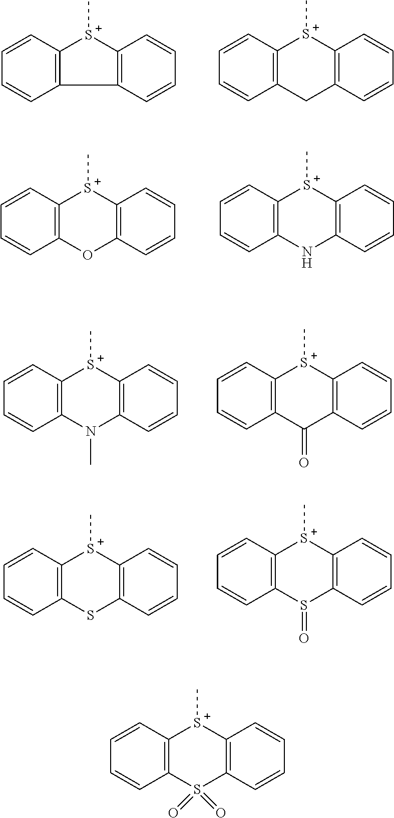

In the formulae, a broken line designates a point of attachment.

In the formula (Z-4), L^{A} and L^{B} are each independently a single bond, an ether bond, an ester bond, an amide bond, a sulfonate ester bond, a sulfonamide bond, a carbonate bond, or a carbamate bond. Among them, L^{A} is preferably a single bond, an ether bond, an ester bond, or a sulfonate ester bond, and more preferably an ester bond or a sulfonate ester bond. L^{B} is preferably a single bond, an ether bond, or an ester bond, and more preferably a single bond.

In the formula (B), X^{L} is a single bond or a C₁-C₄₀ hydrocarbylene group which may contain a heteroatom. The hydrocarbylene group may be straight, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the C₁-C₄₀ hydrocarbylene group which may contain a heteroatom of X^{L} are shown below, but not limited thereto. In the following formulae. * each designates a point of attachment to L^{A} or L^{B}.

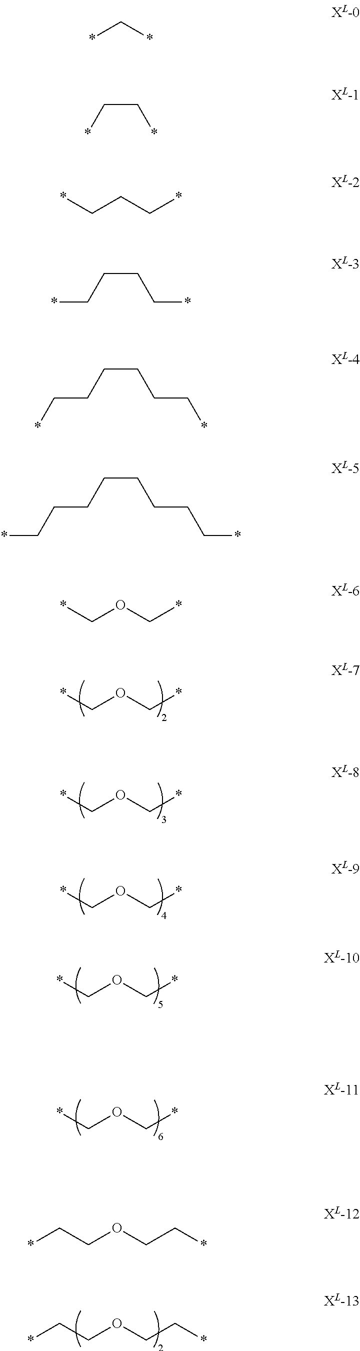

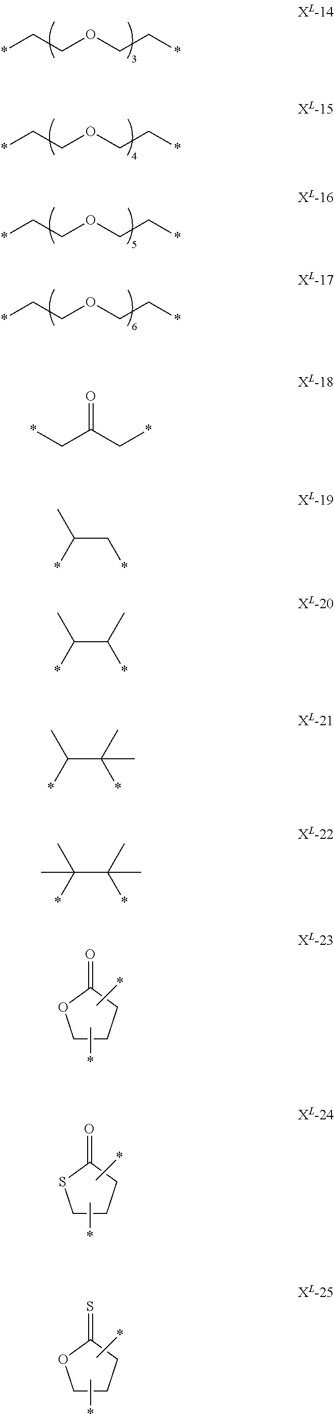

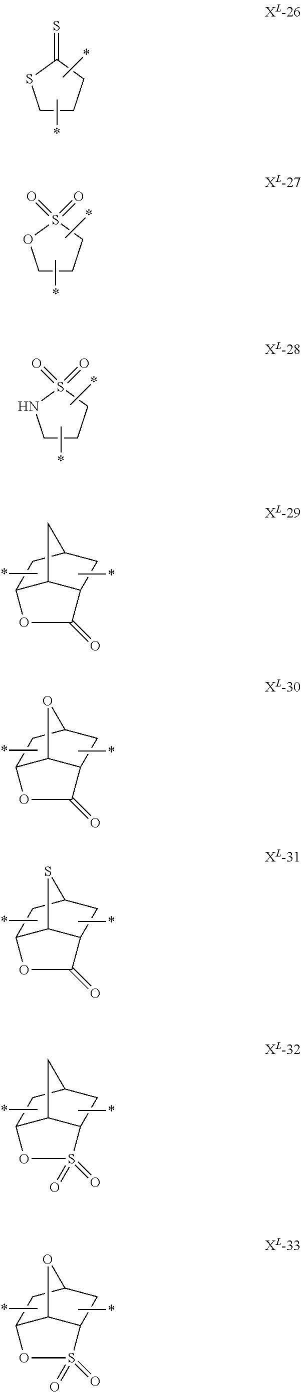

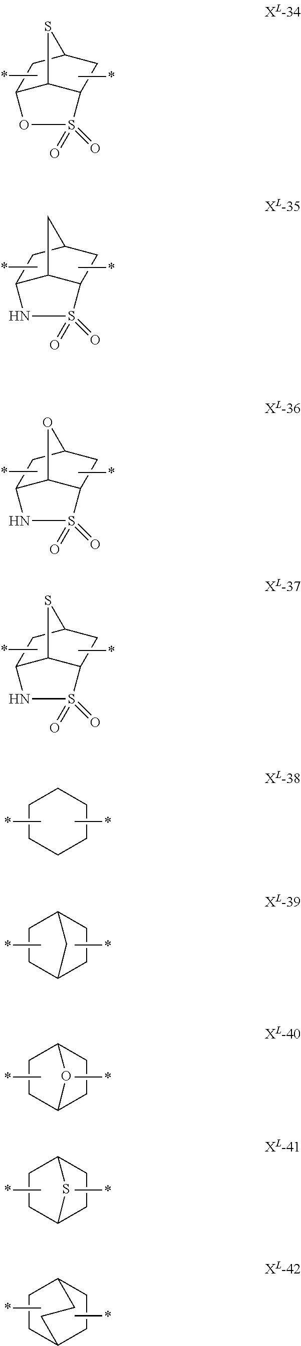

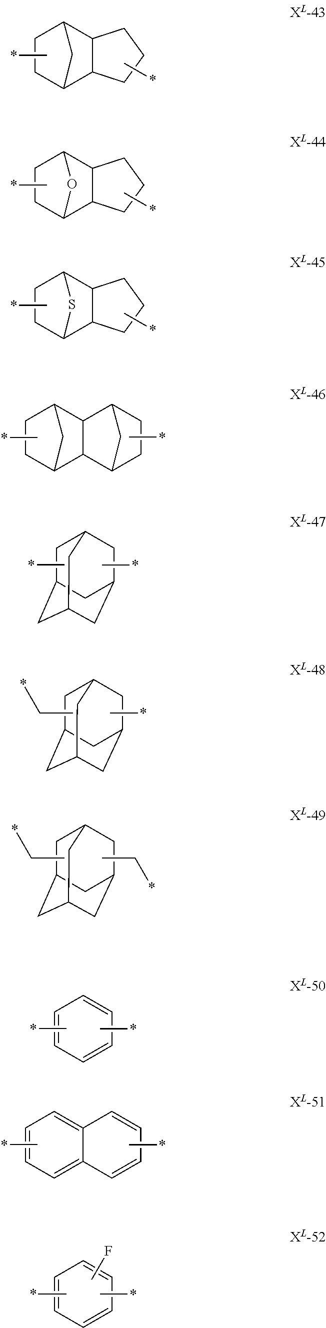

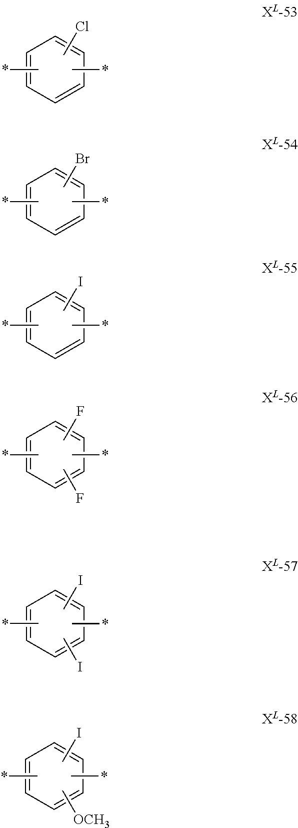

Among them, X^{L}-0 to X^{L}-22, X^{L}-29 to X^{L}-34, and X^{L}-47 to X¹-58 are preferred. As the sulfonium cation having the formula (Z-4), a sulfonium cation having the formula (Z-4-1) is preferred.

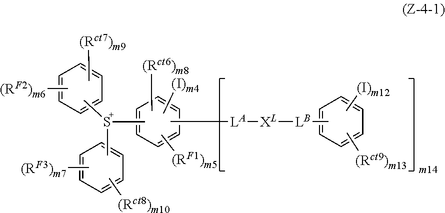

In the formula, m4 to m10, m12 to m14, R^{F1} to R^{F3}, Rot6 to R^{ct9}, L^{A}, L^{B}, and X^{L} are as defined above.

As the cation having the formula (Z-4-1), a cation having the formula (Z-4-2) is preferred.

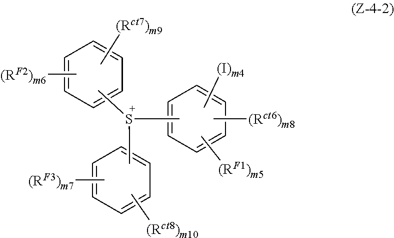

In the formula, m4 to m10, RFI to R^{F3}, and R^{ct6} to R^{ct8} are as defined above.

Specific examples of the sulfonium cation having the formula (Z-4) are shown below. but not limited thereto. In the following formulae. Me is a methyl group.

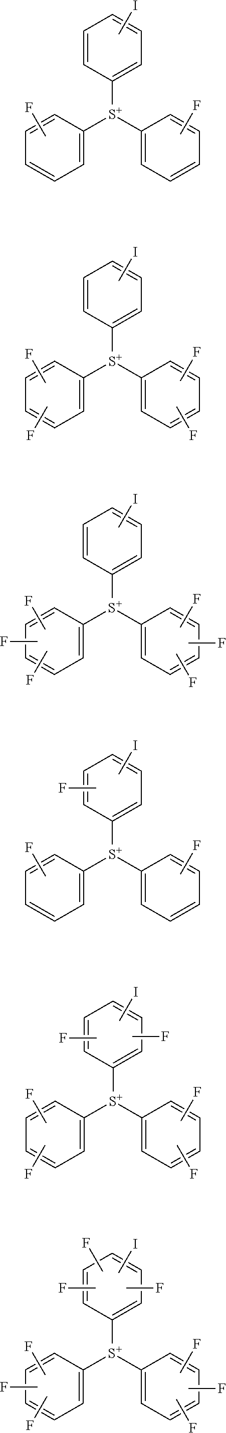

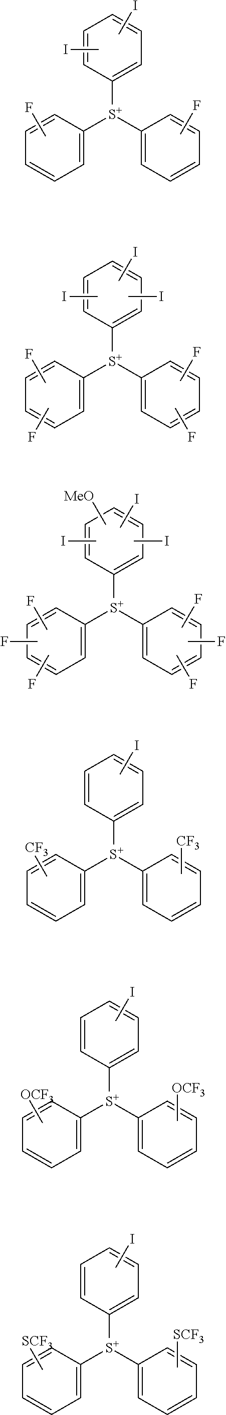

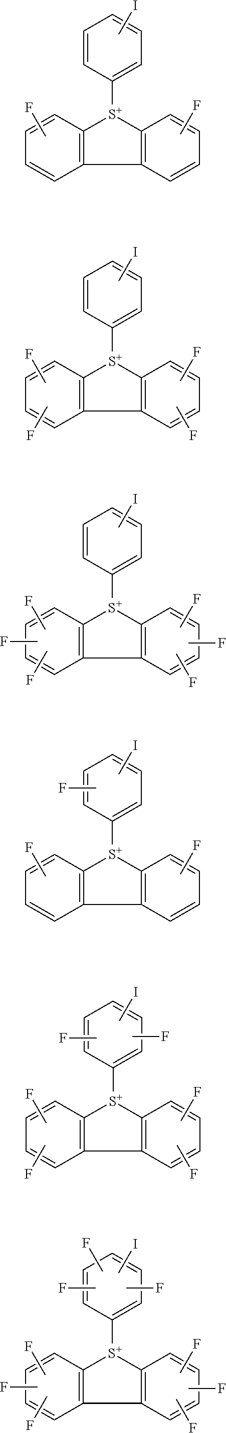

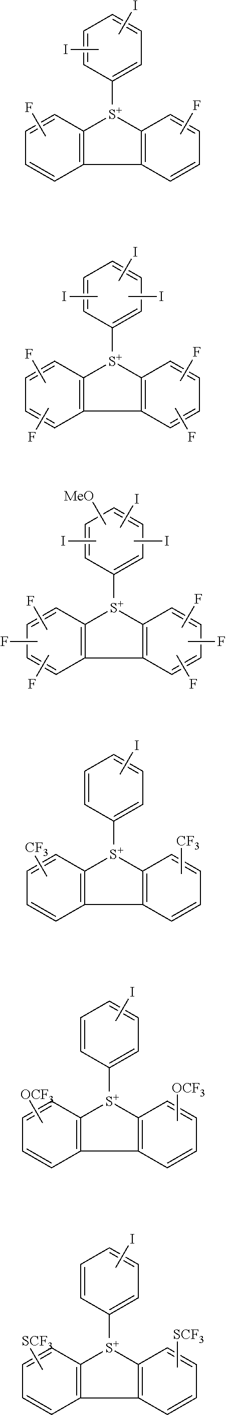

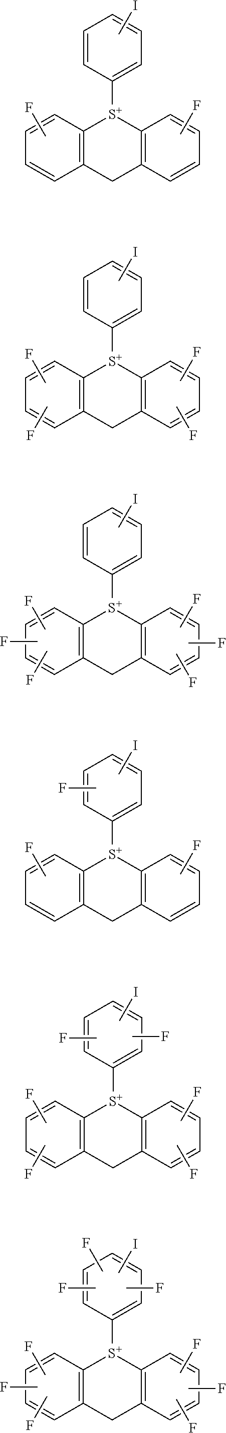

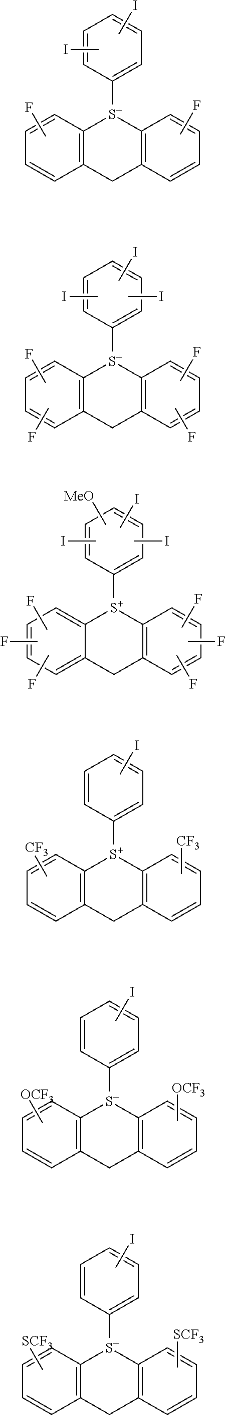

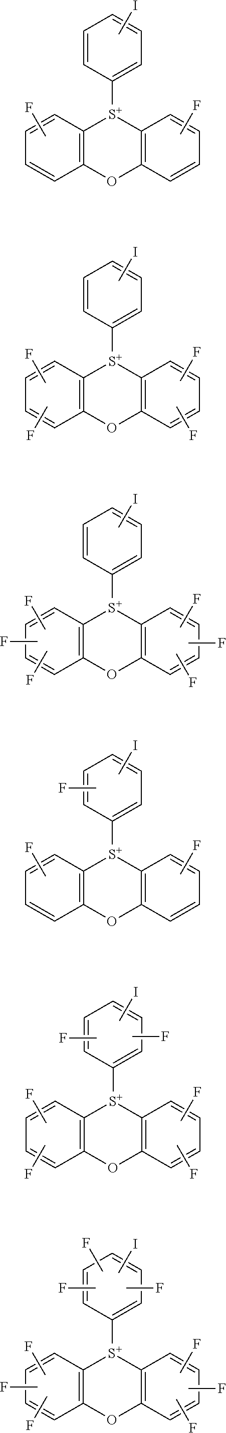

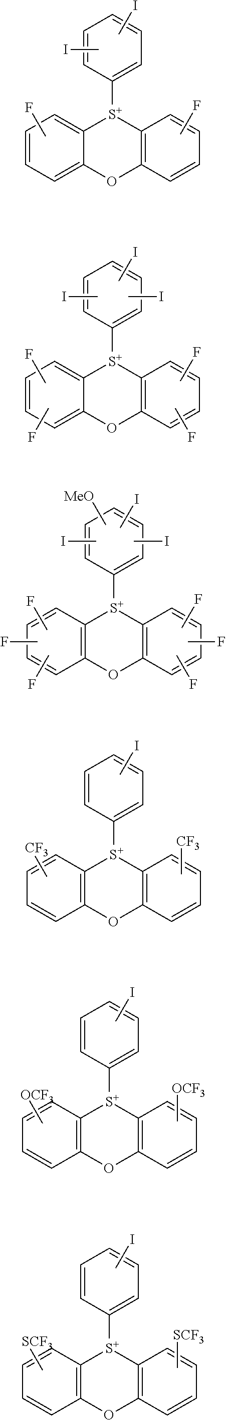

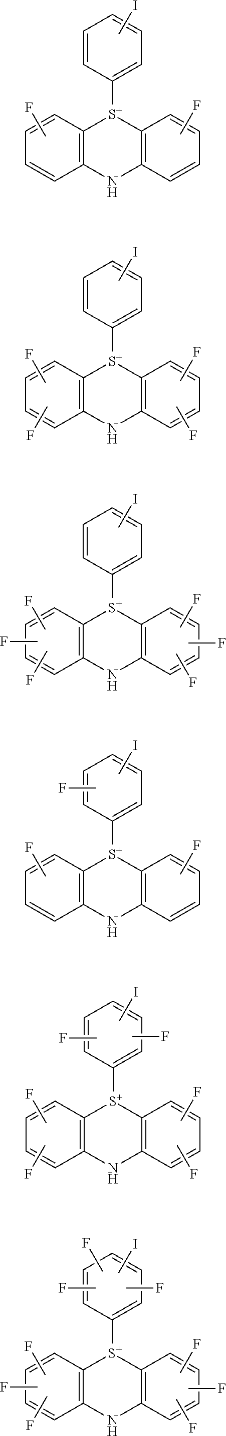

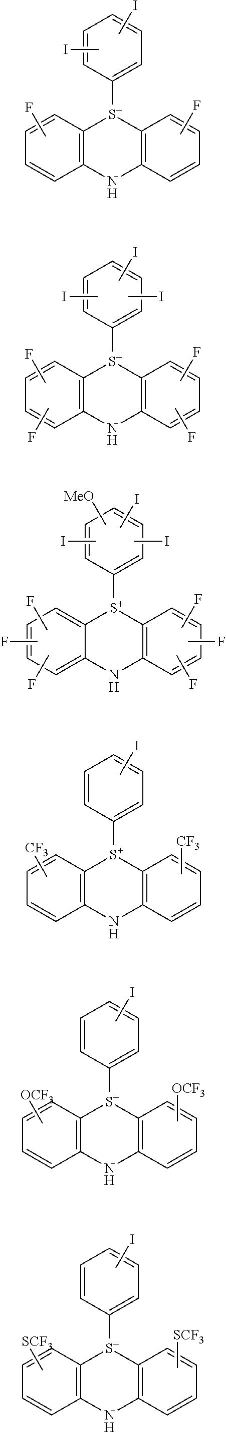

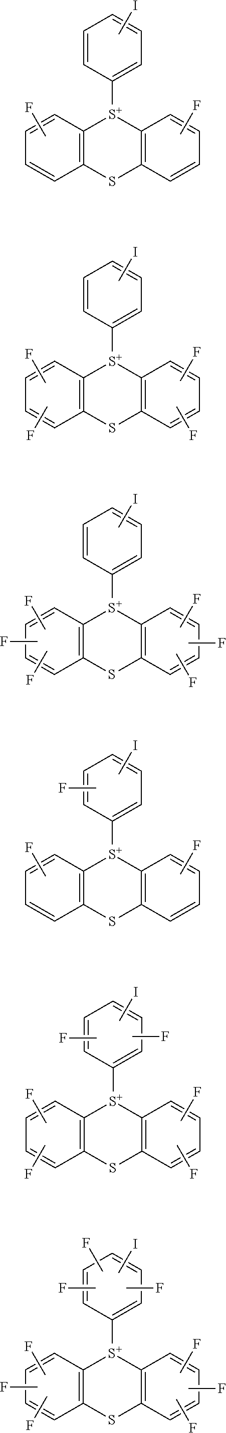

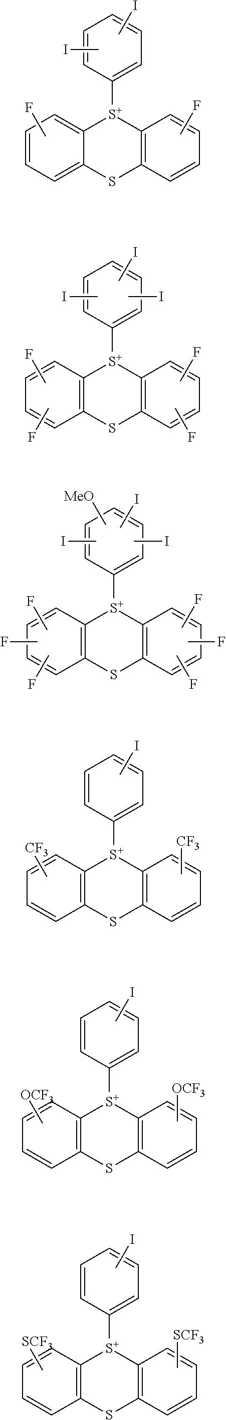

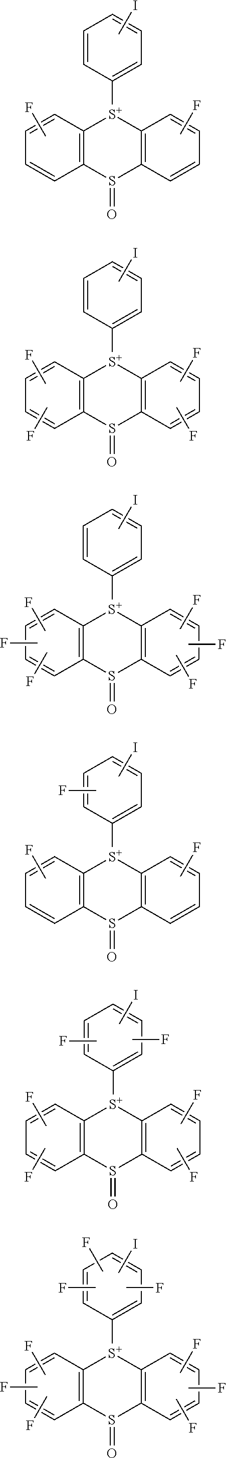

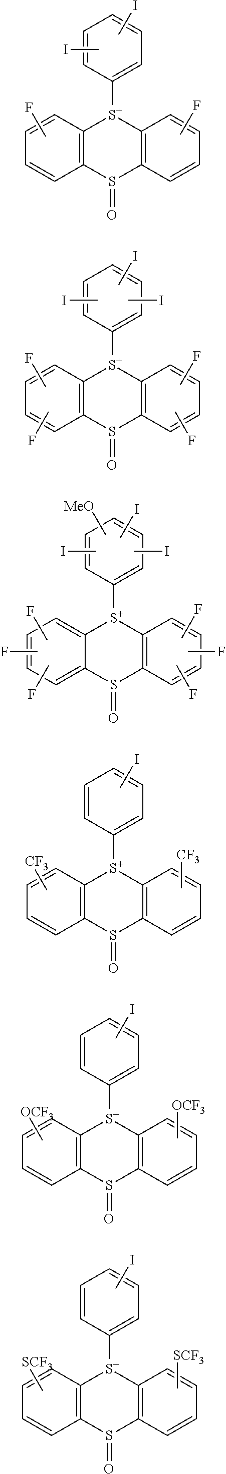

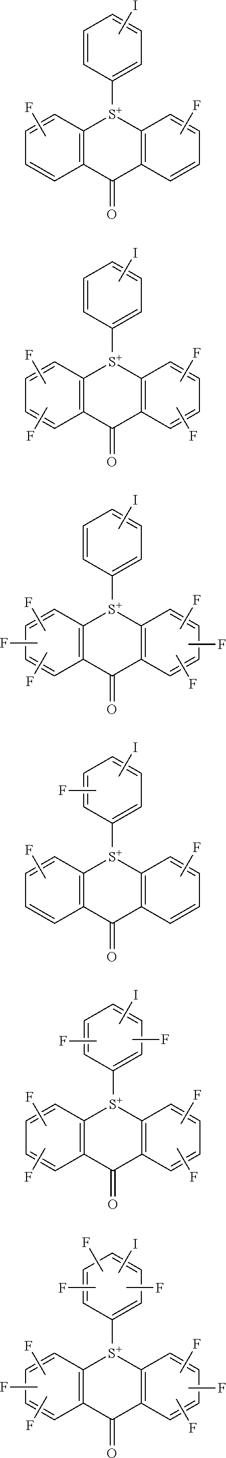

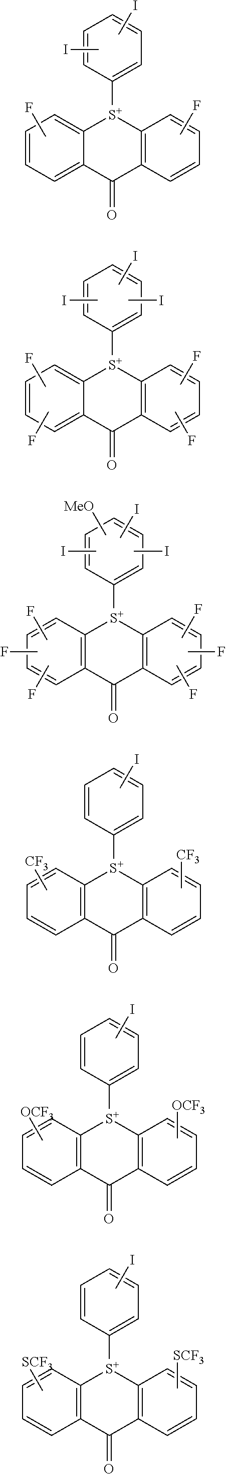

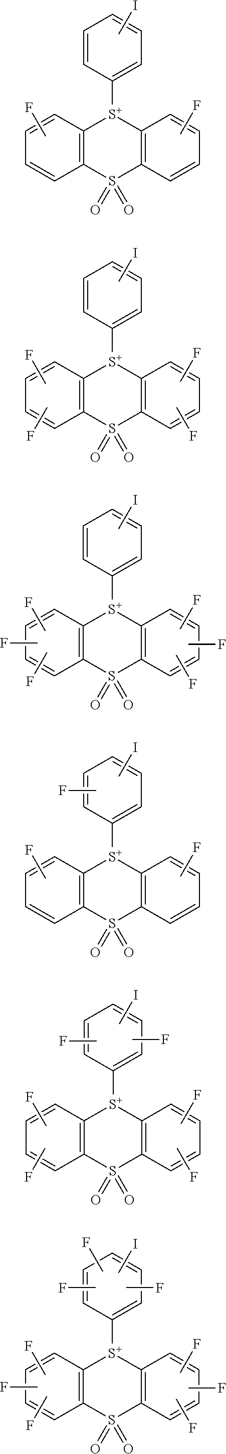

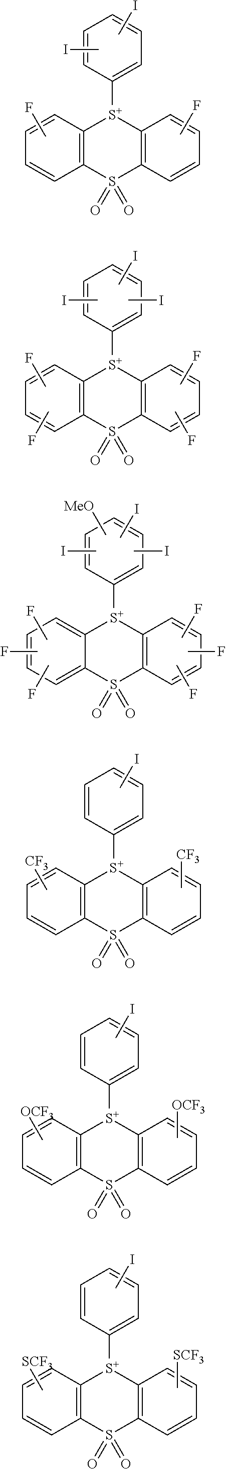

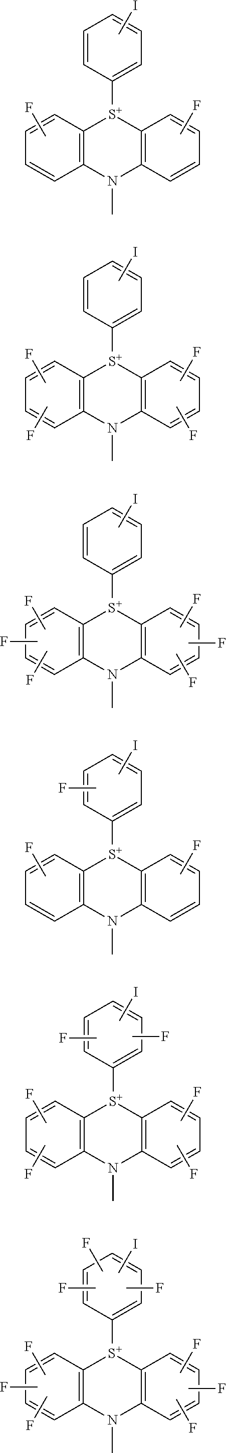

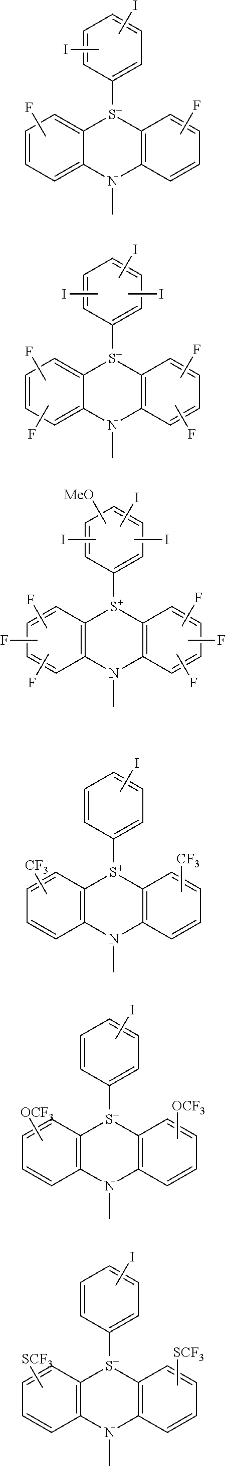

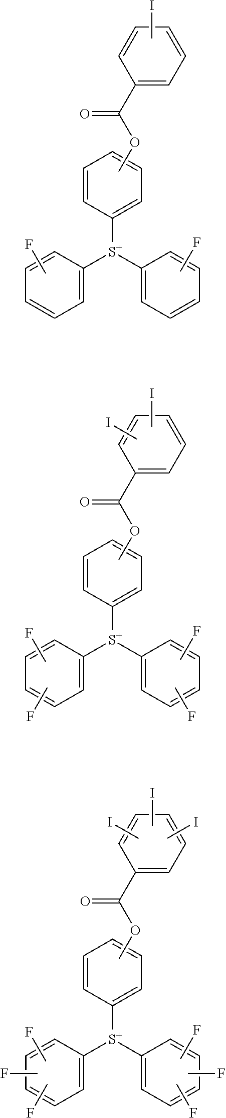

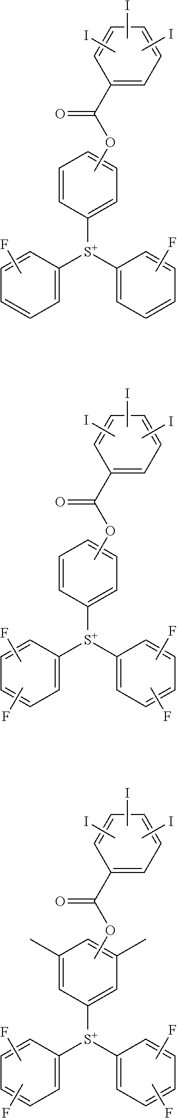

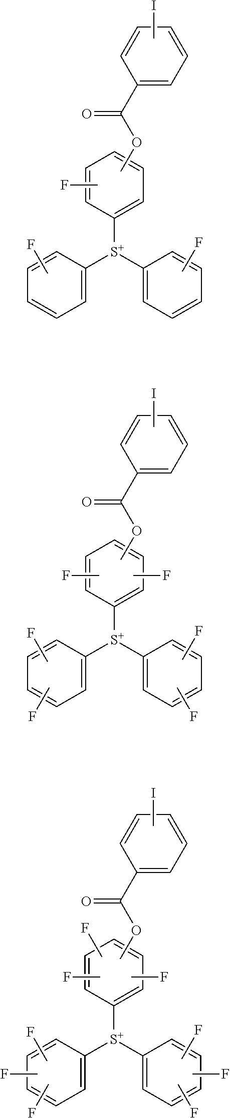

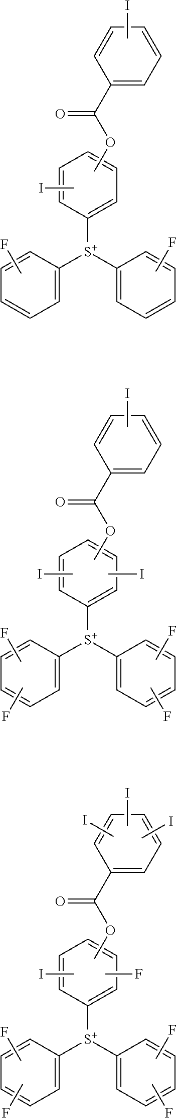

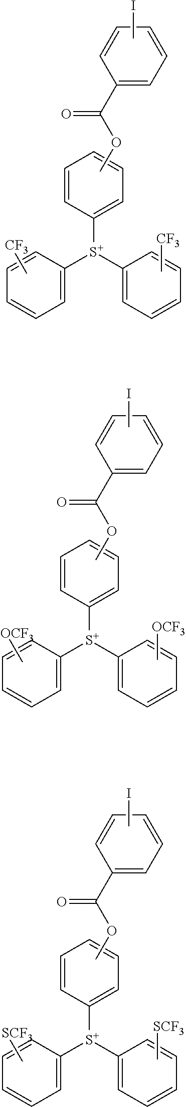

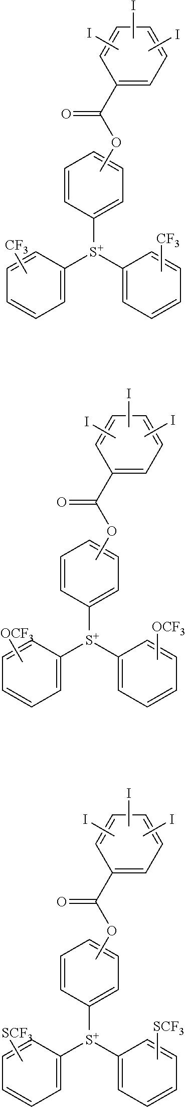

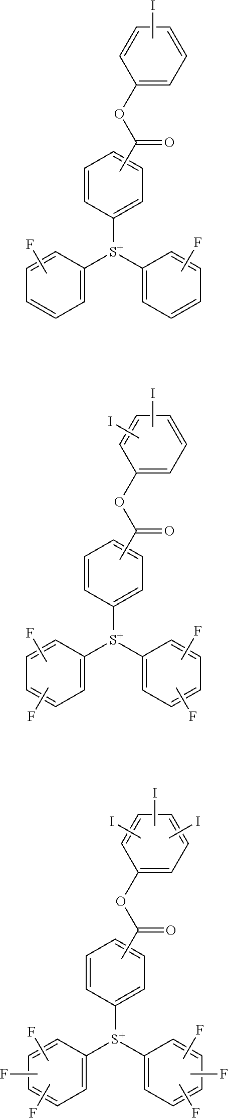

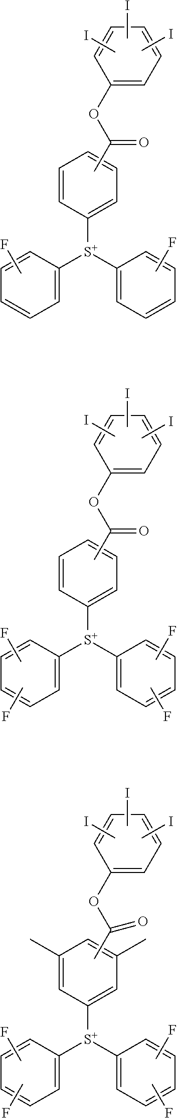

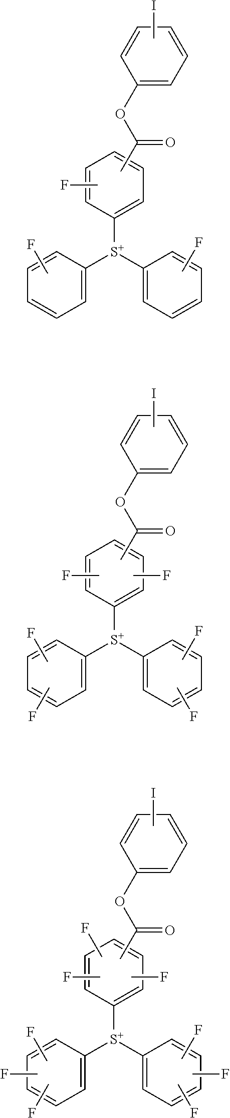

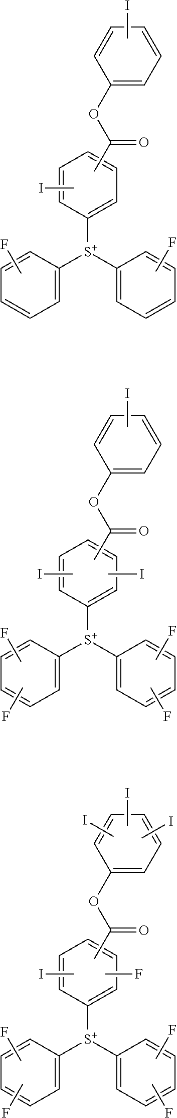

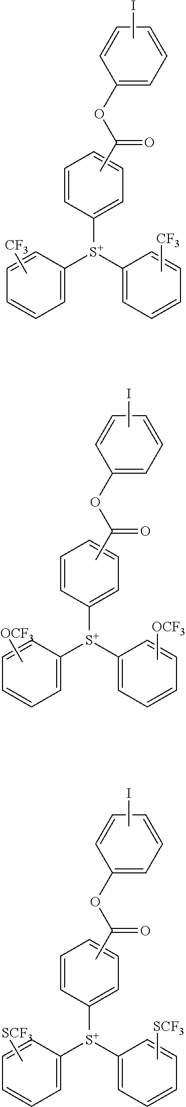

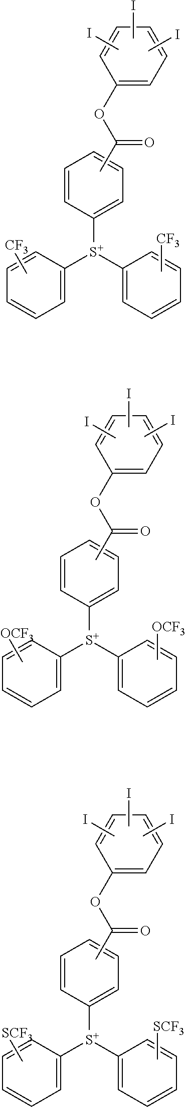

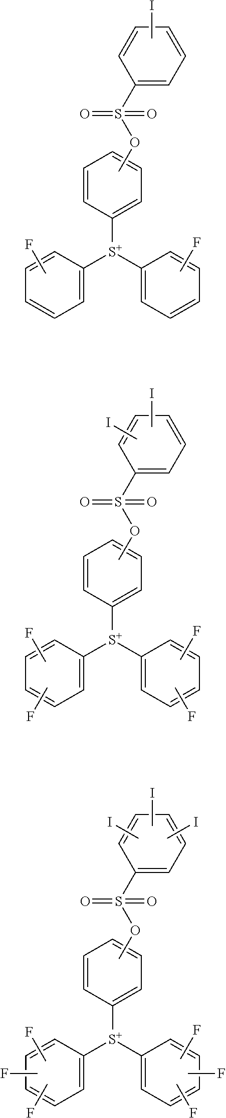

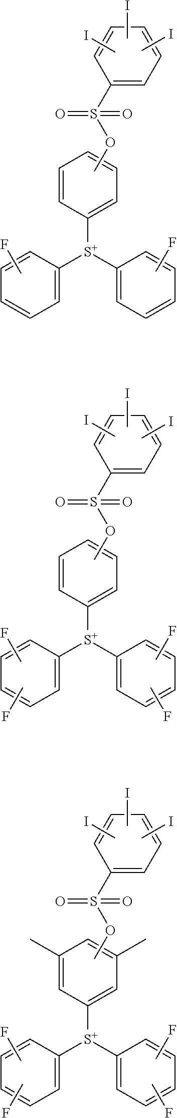

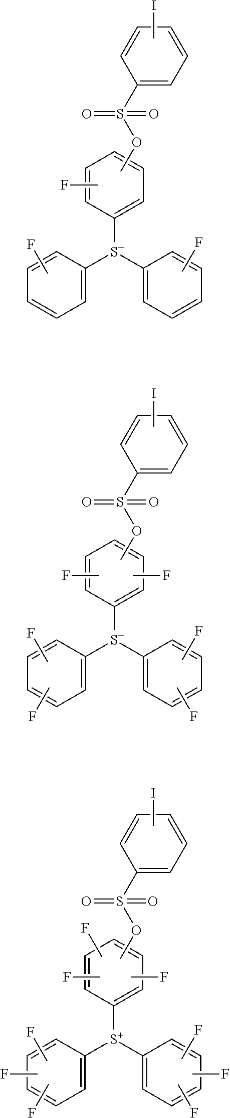

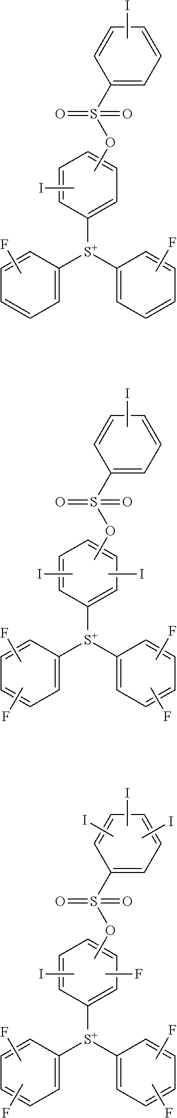

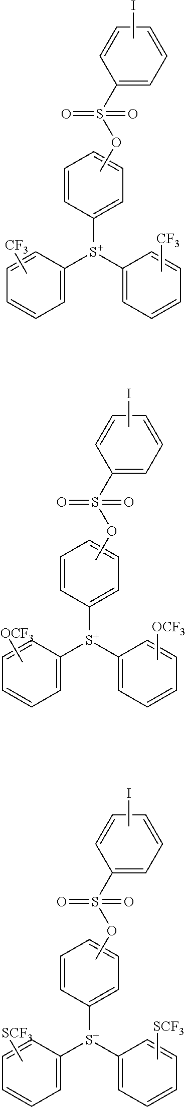

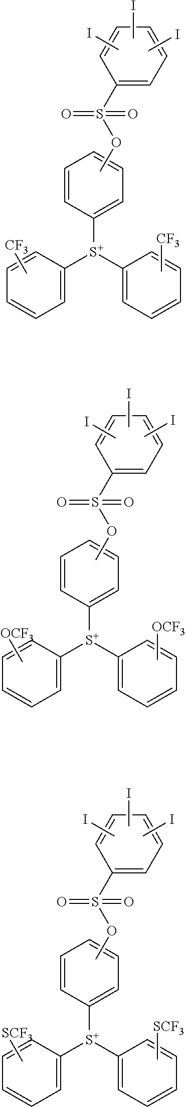

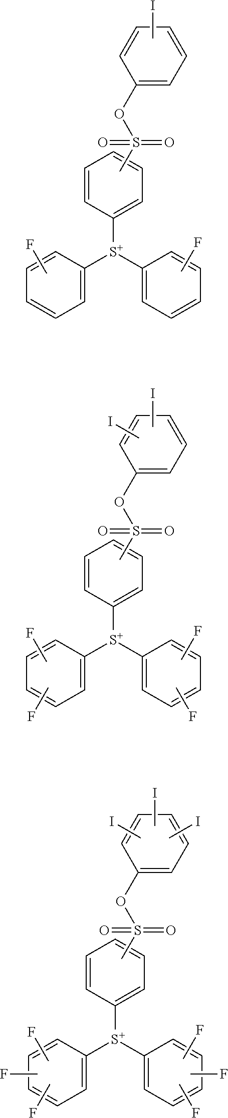

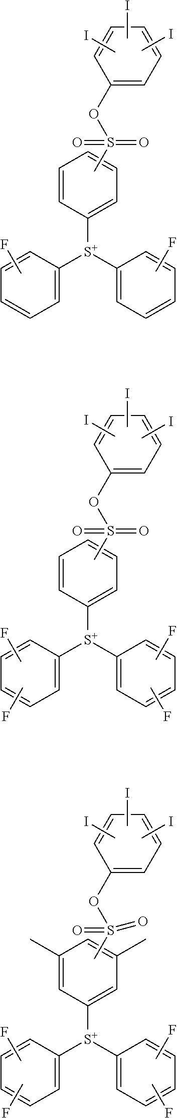

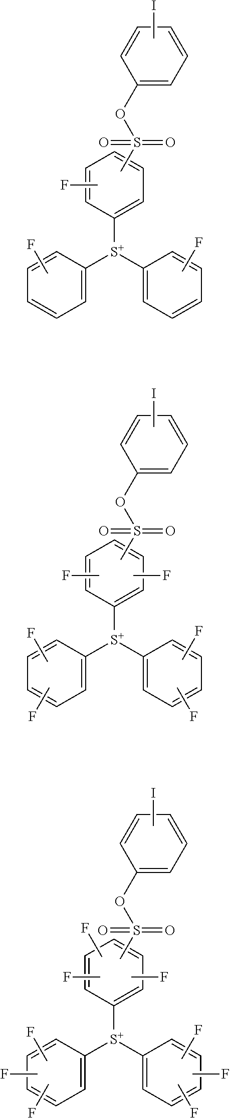

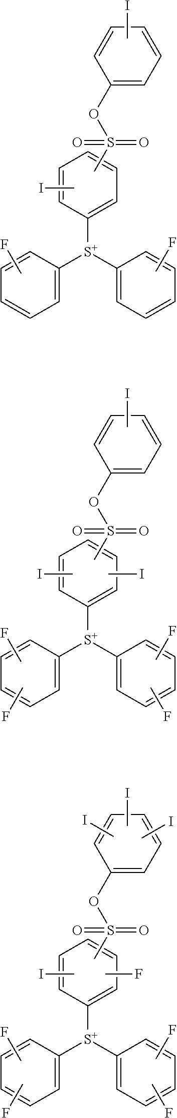

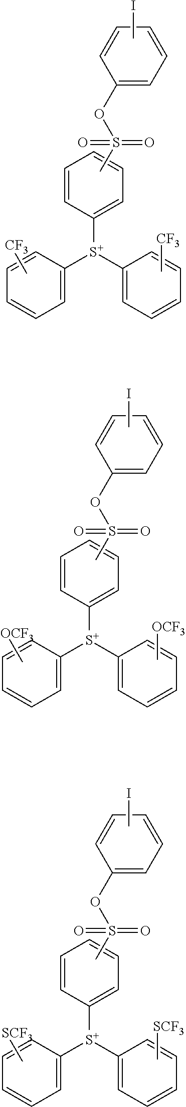

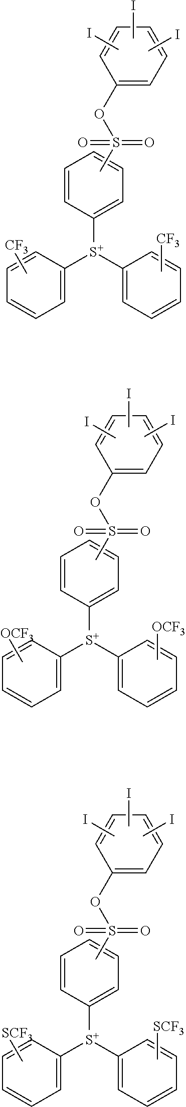

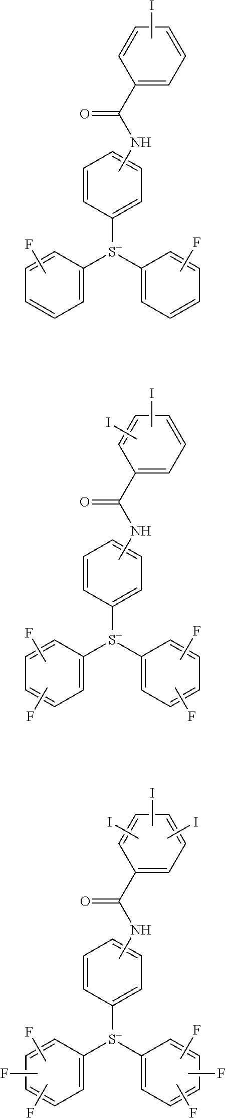

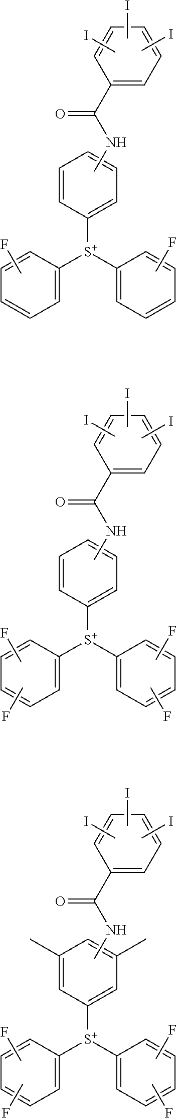

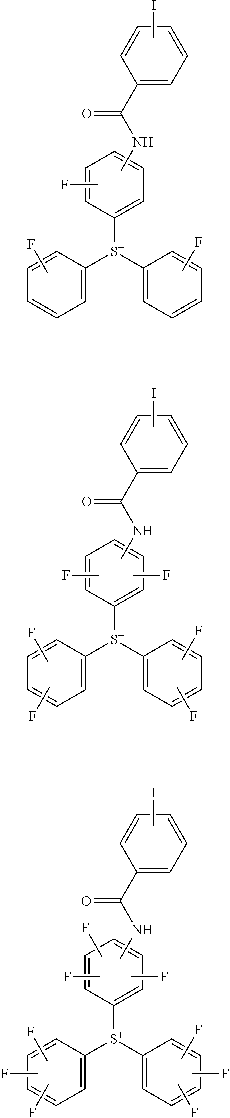

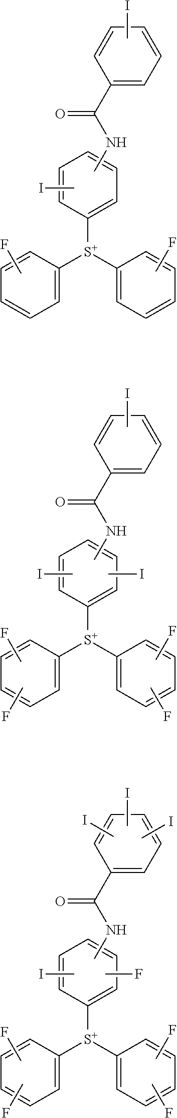

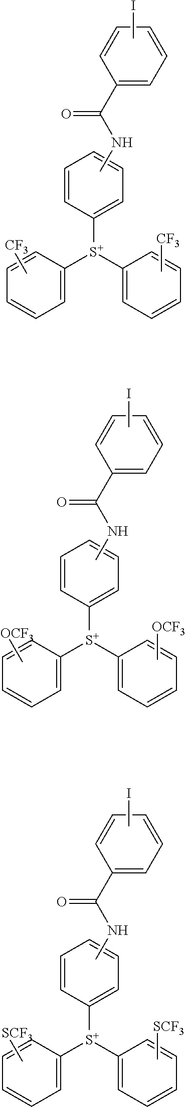

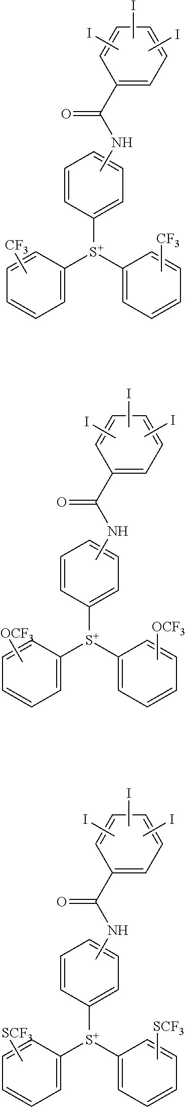

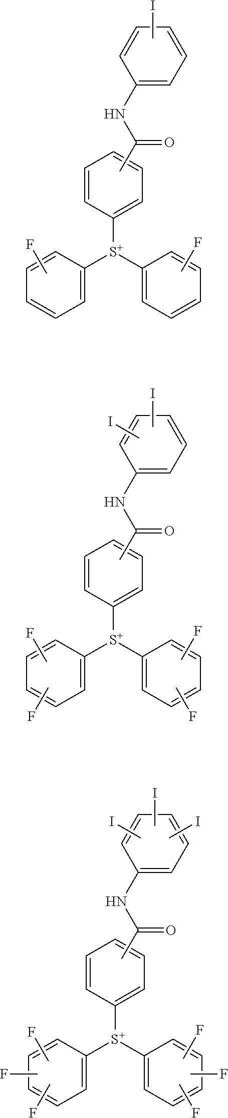

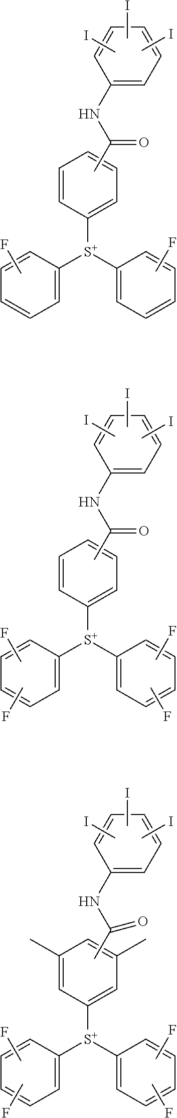

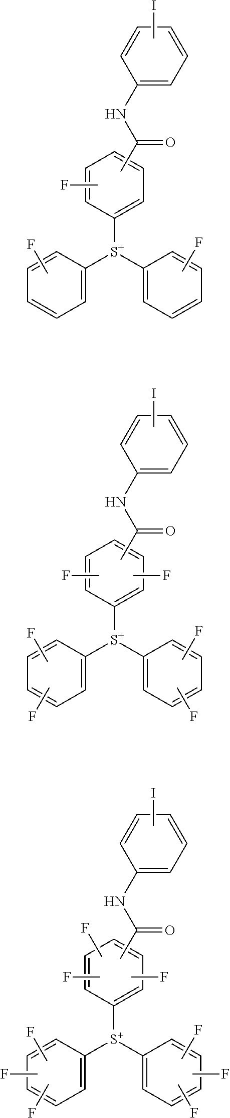

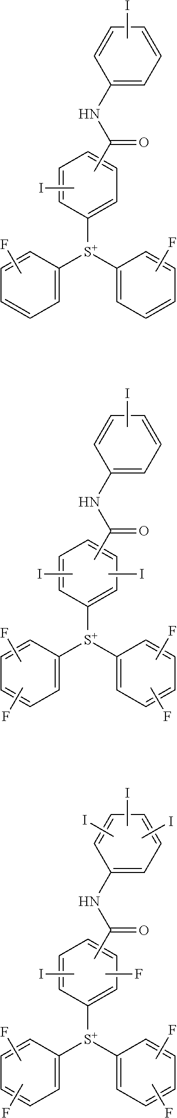

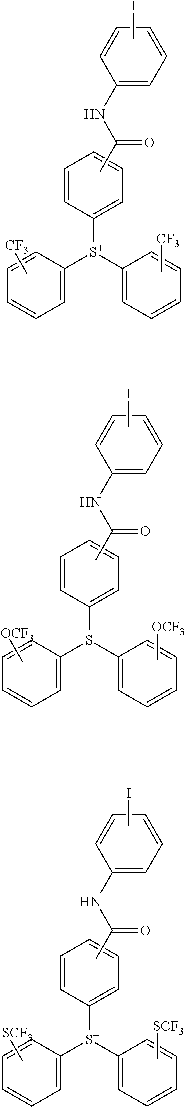

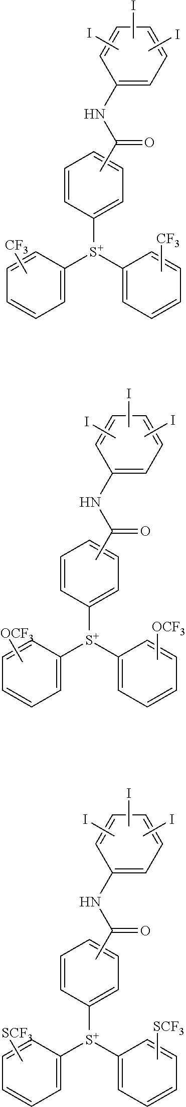

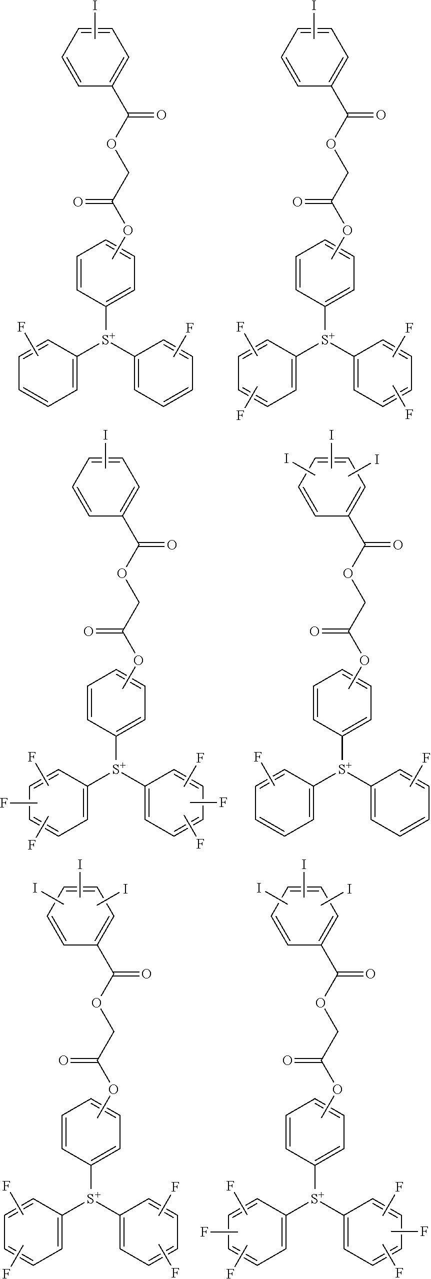

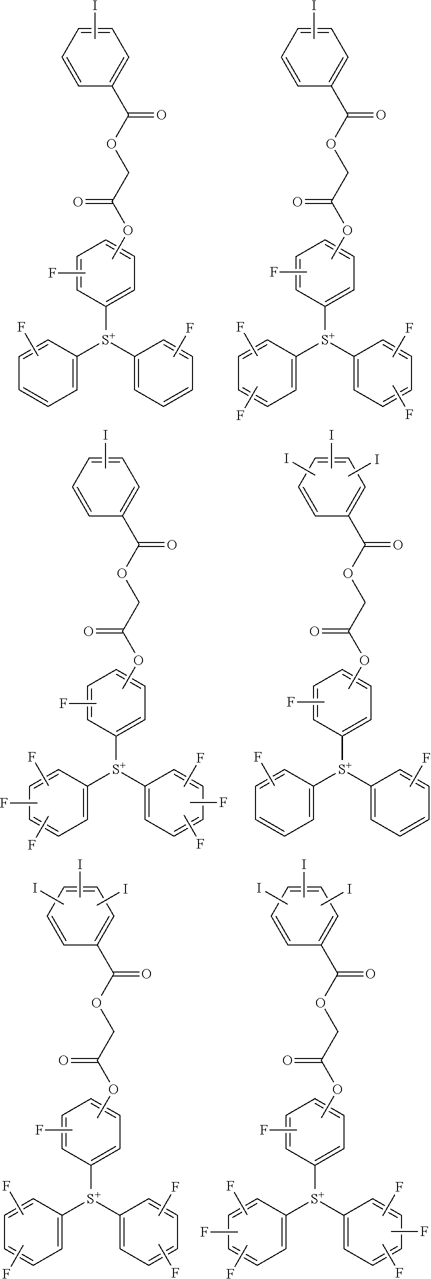

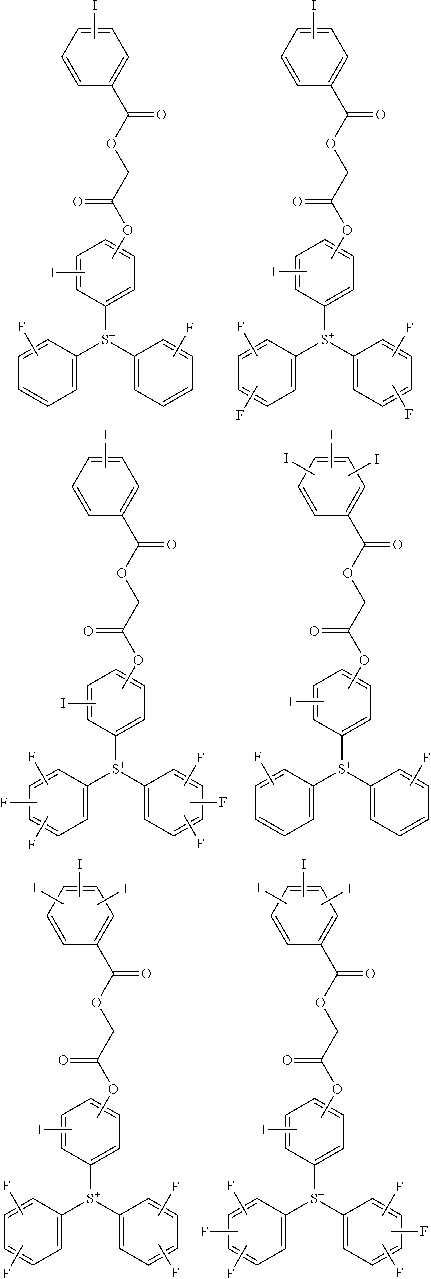

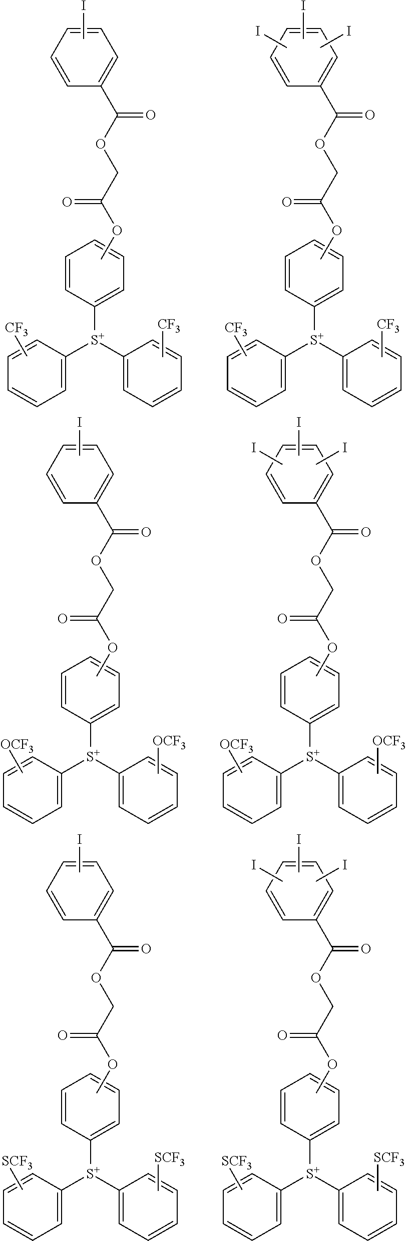

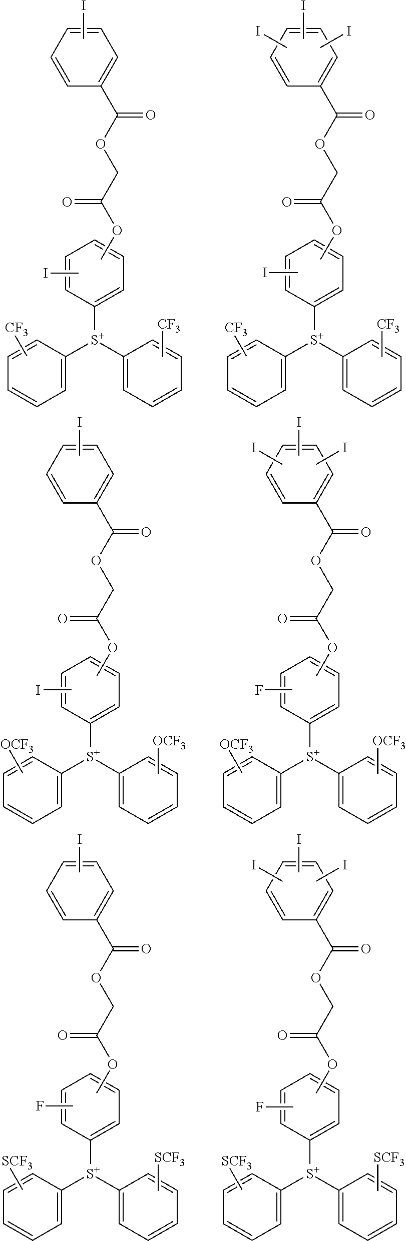

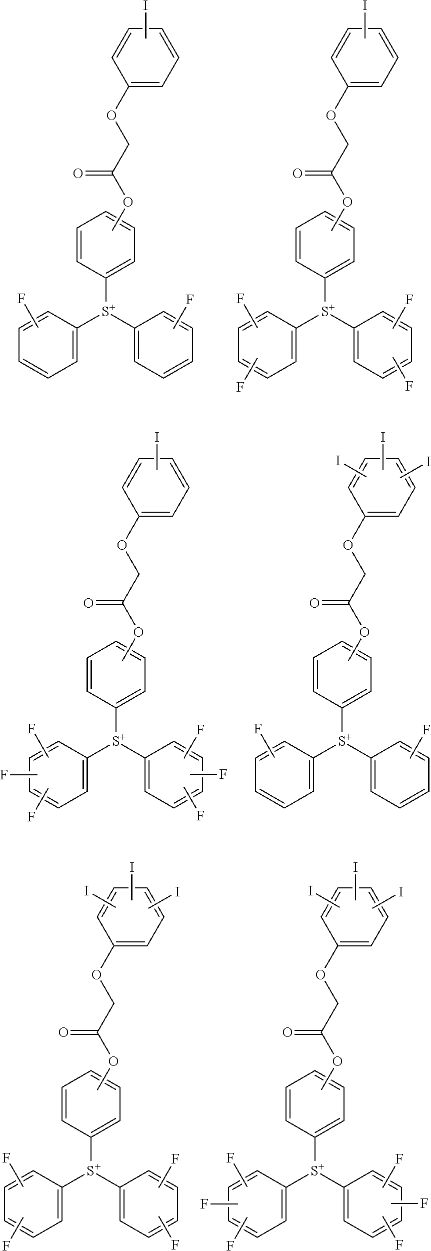

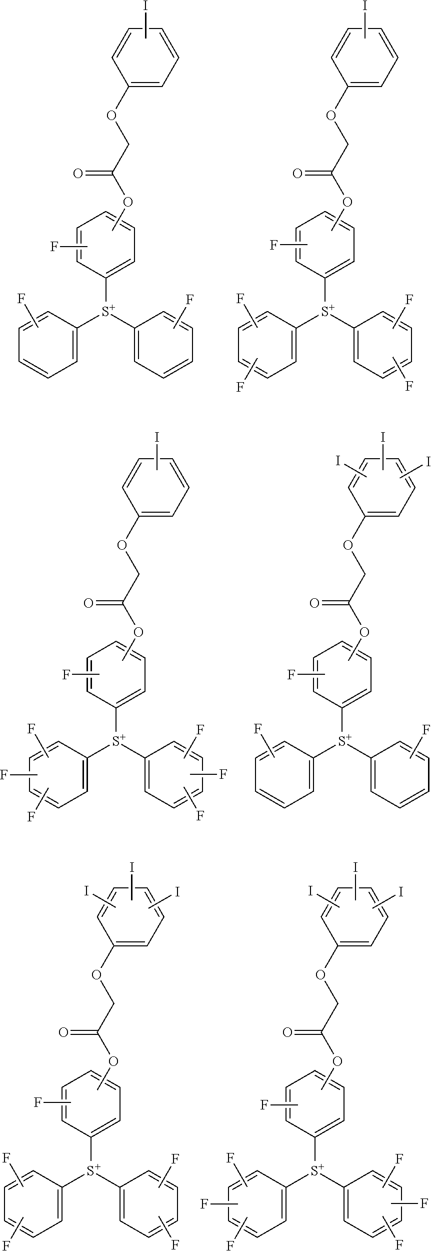

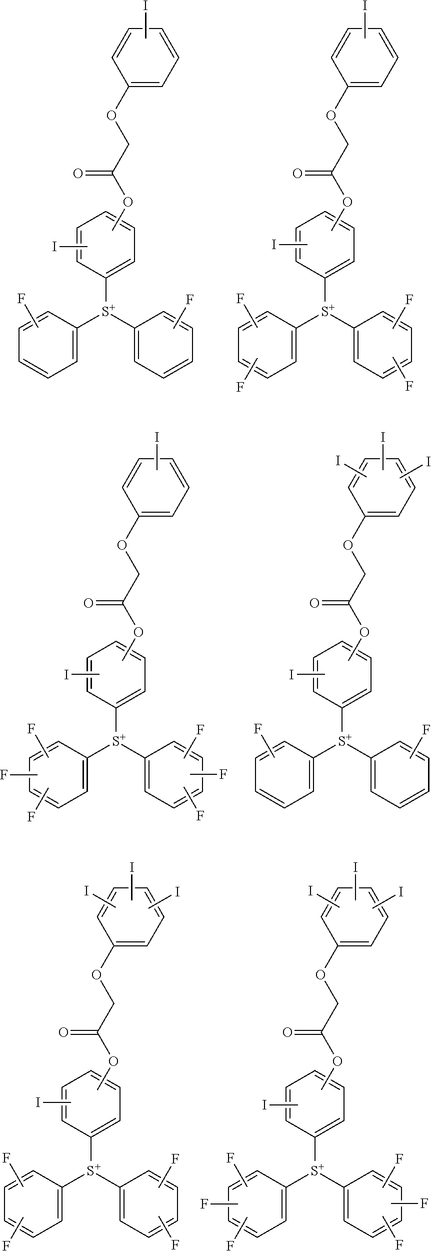

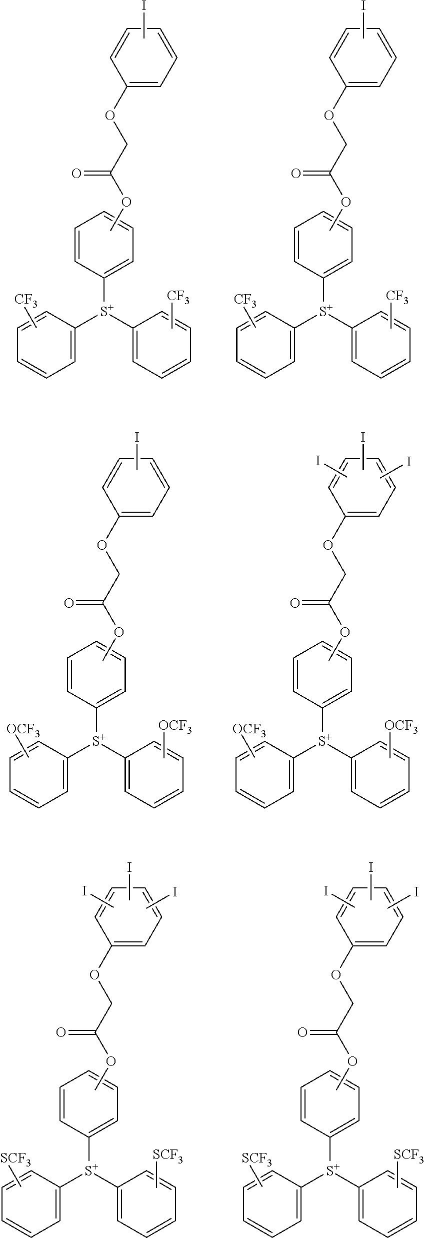

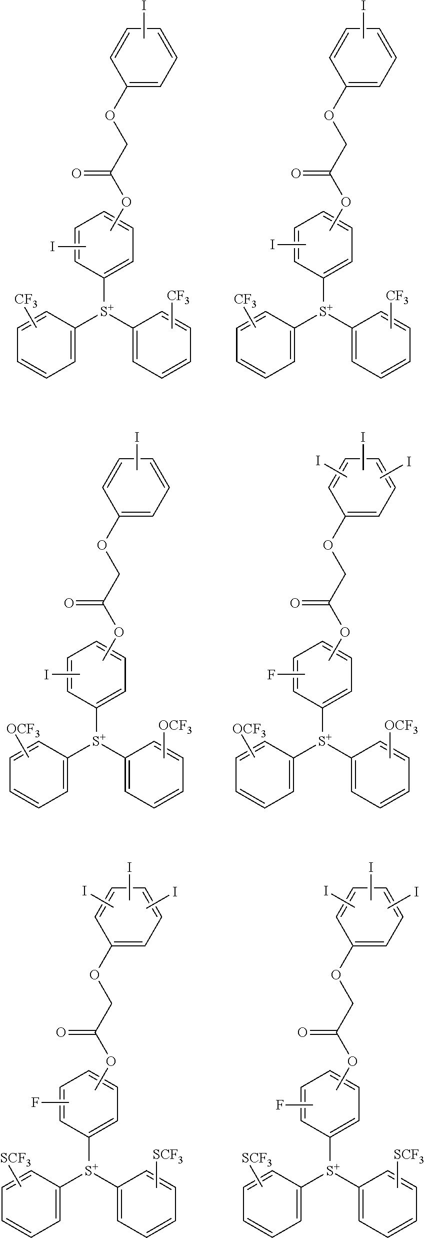

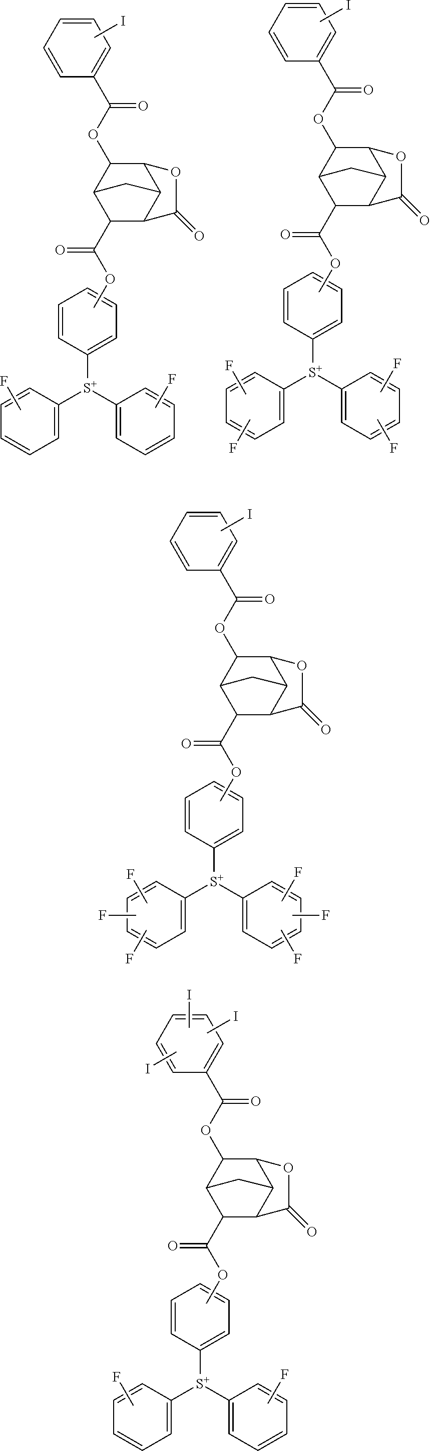

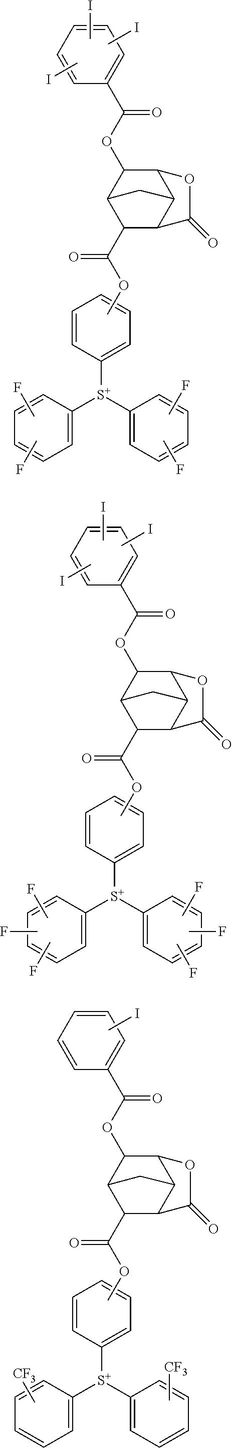

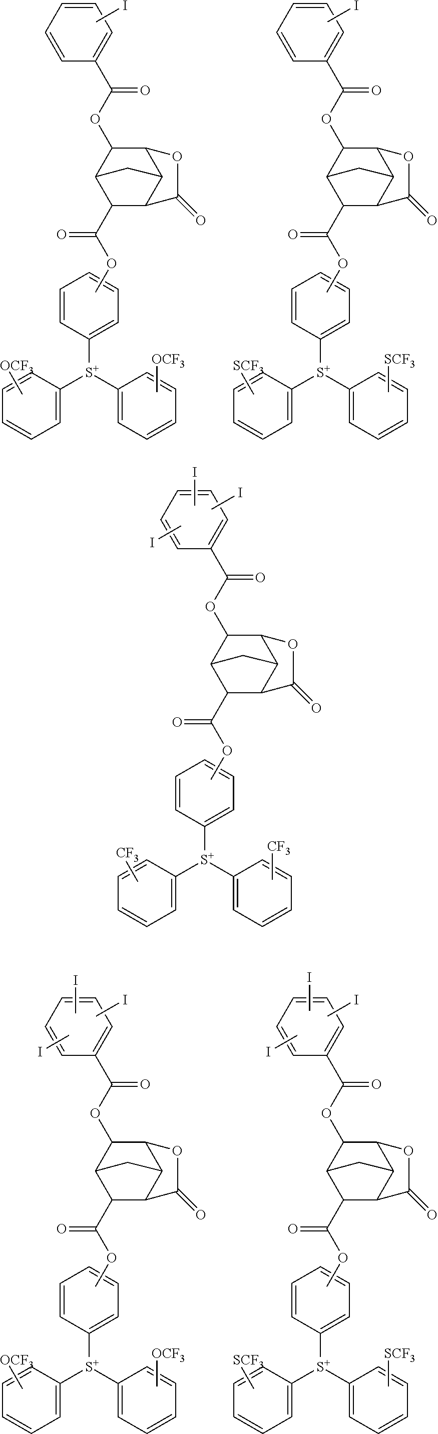

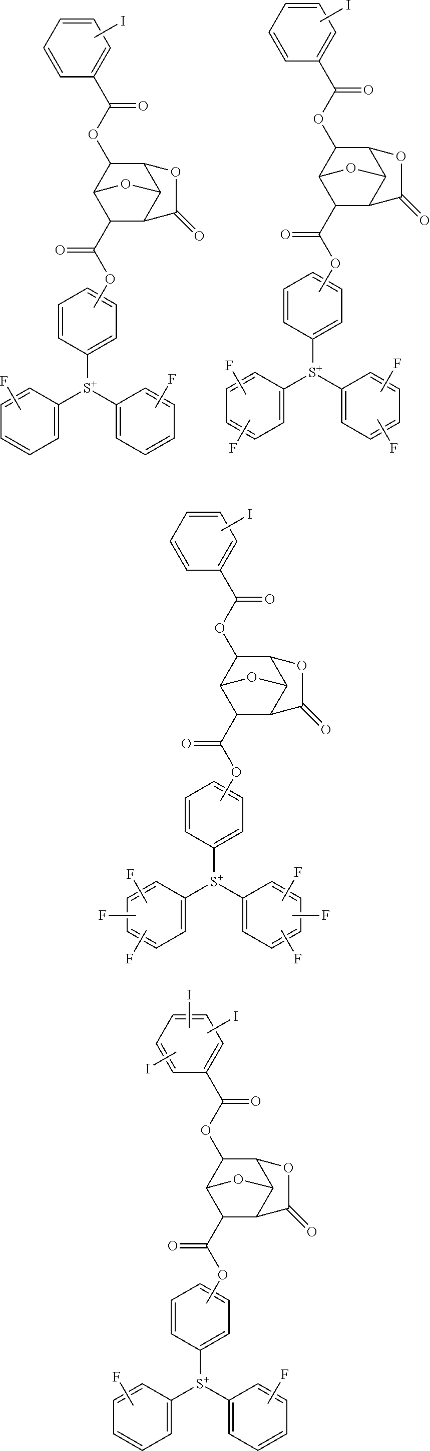

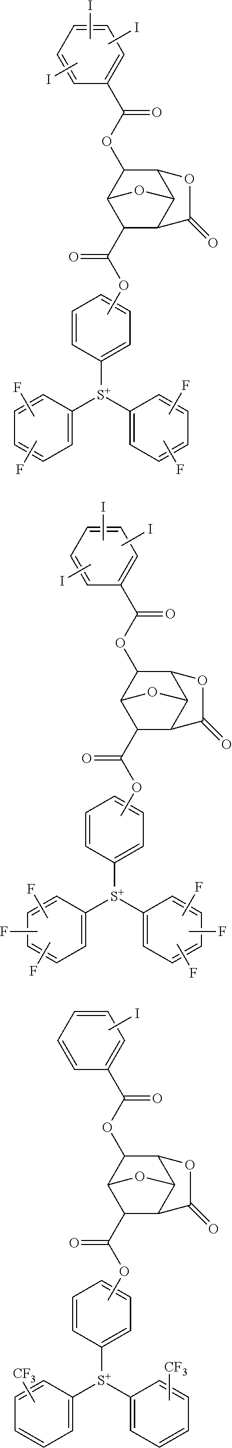

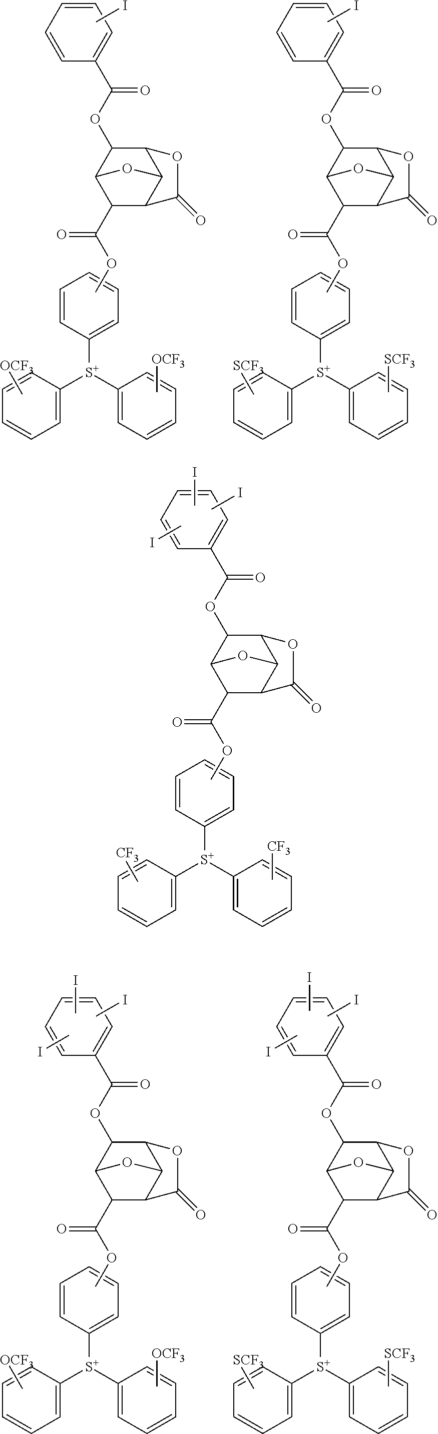

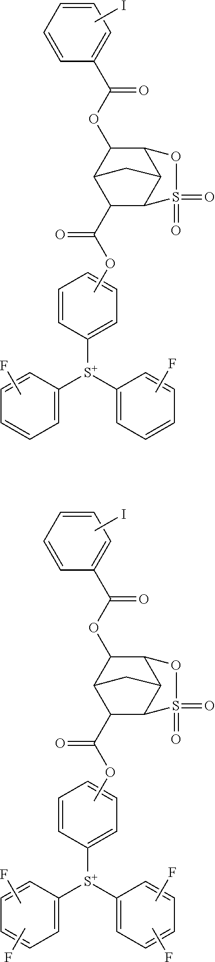

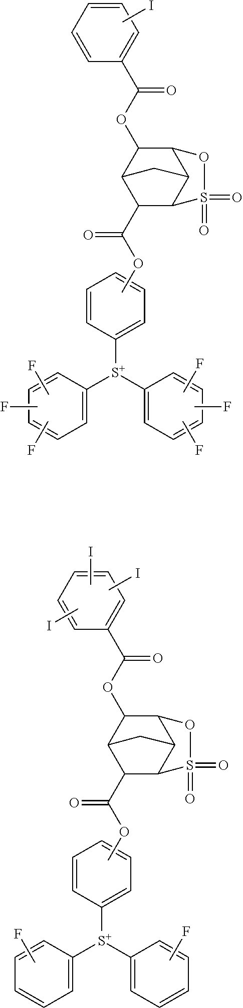

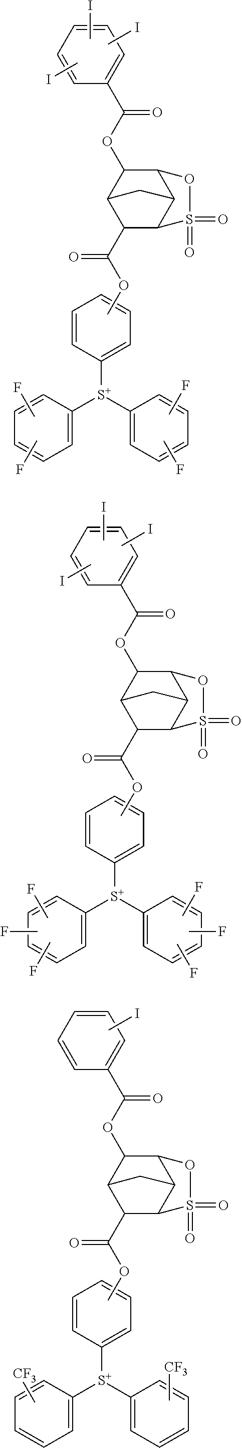

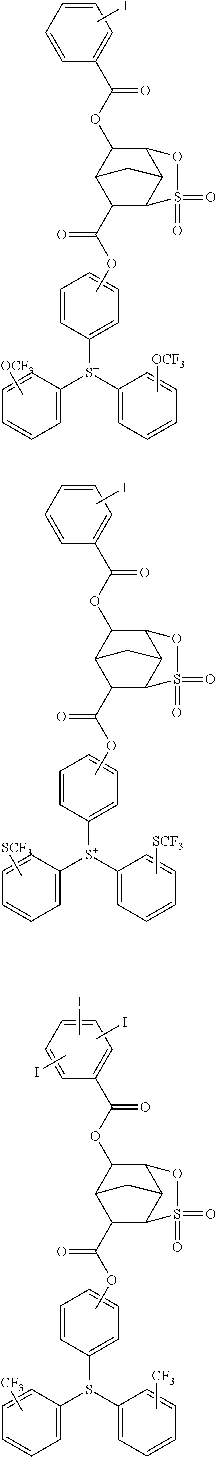

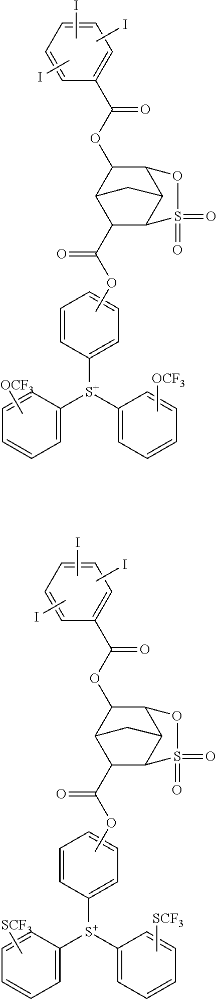

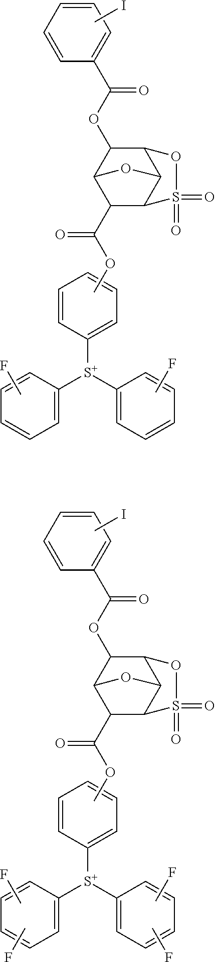

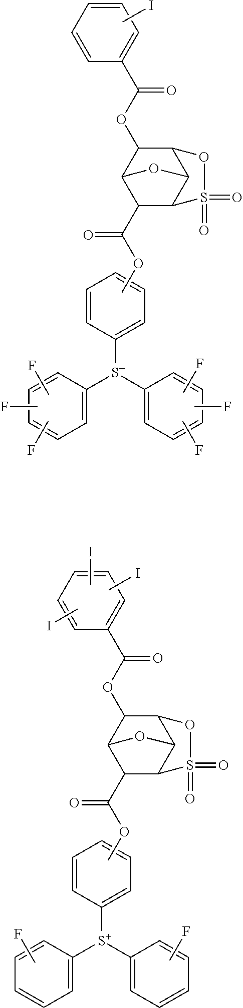

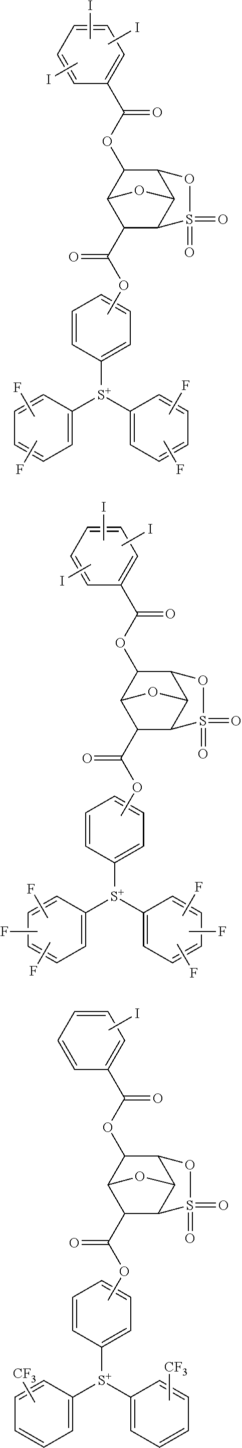

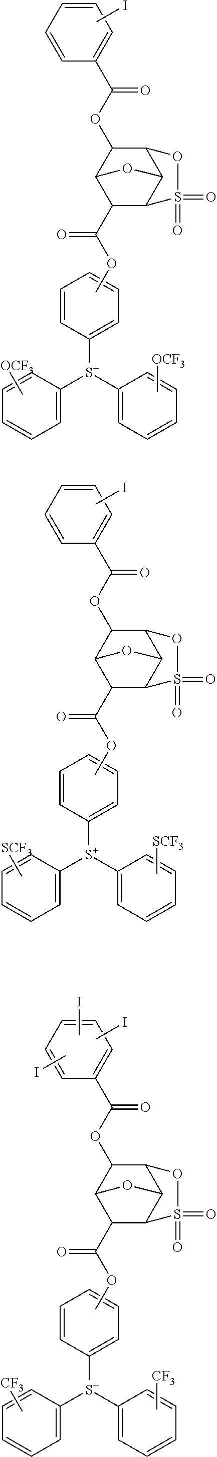

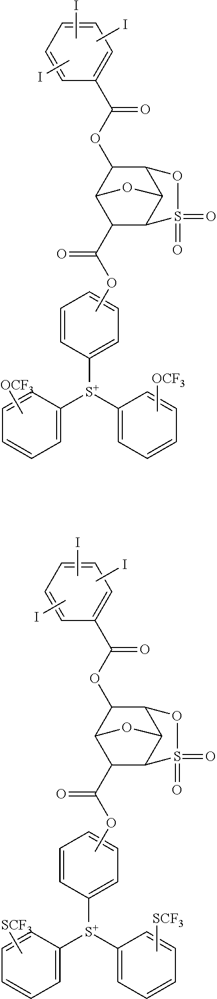

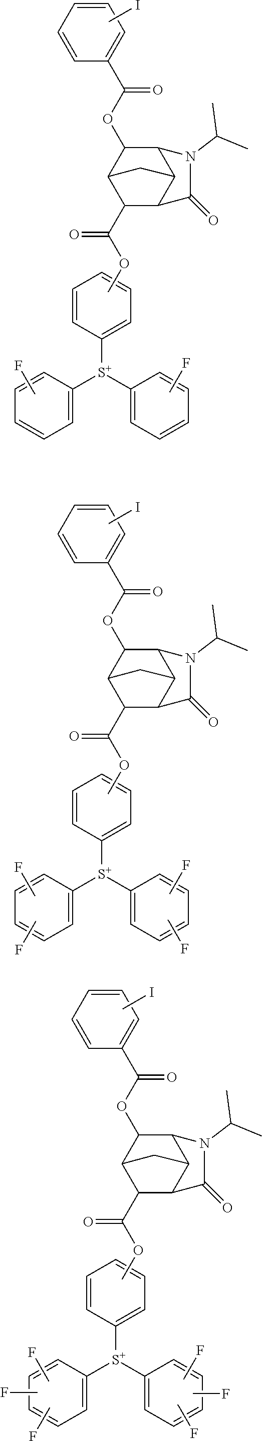

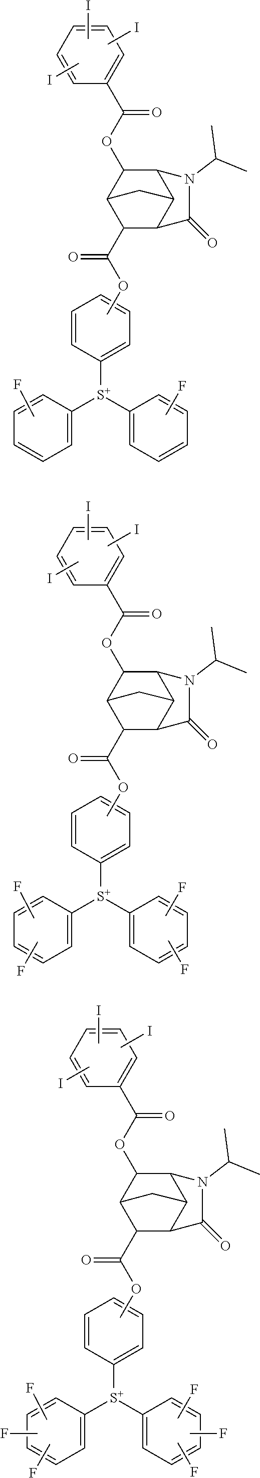

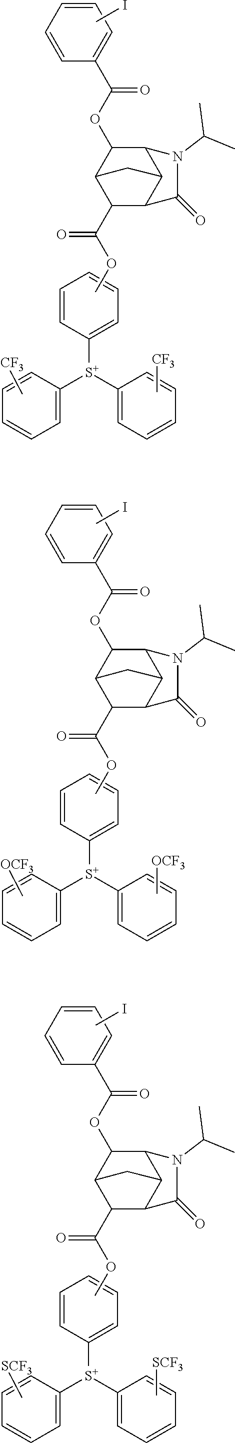

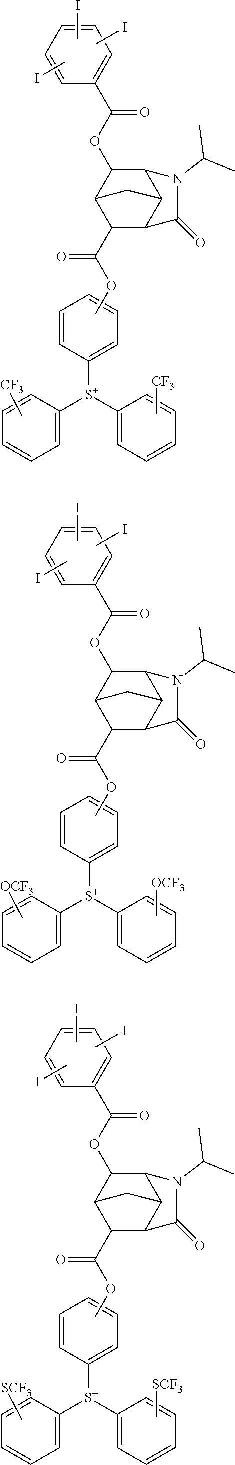

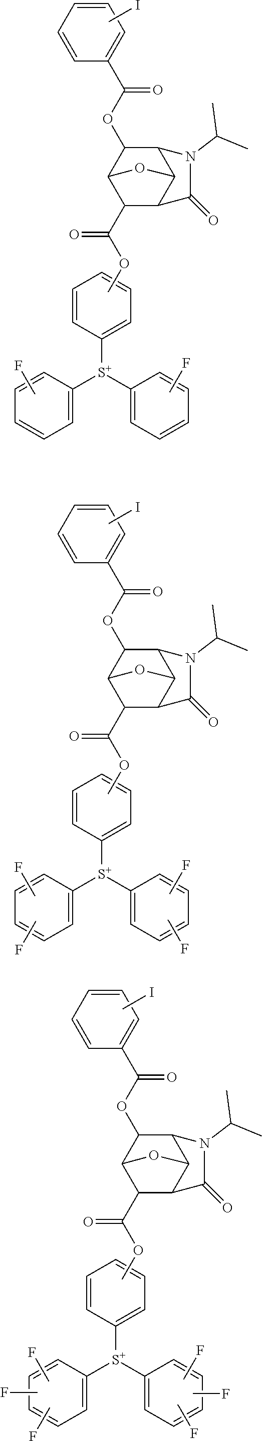

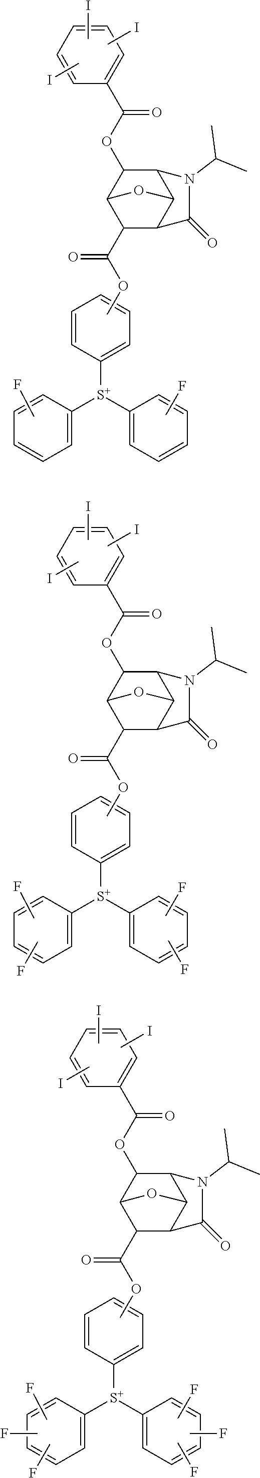

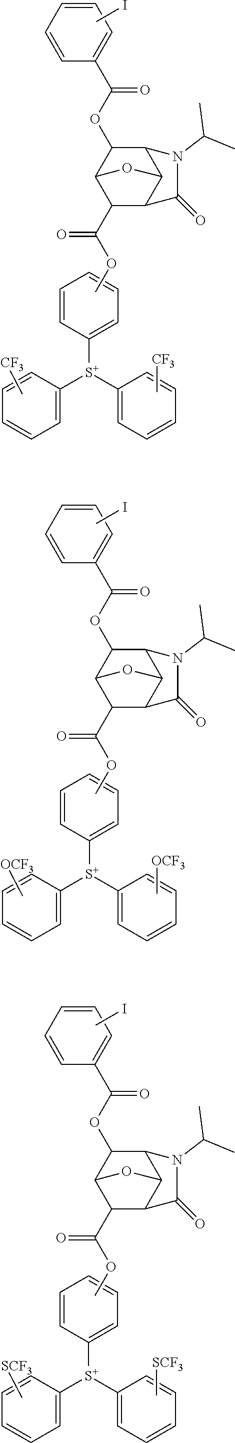

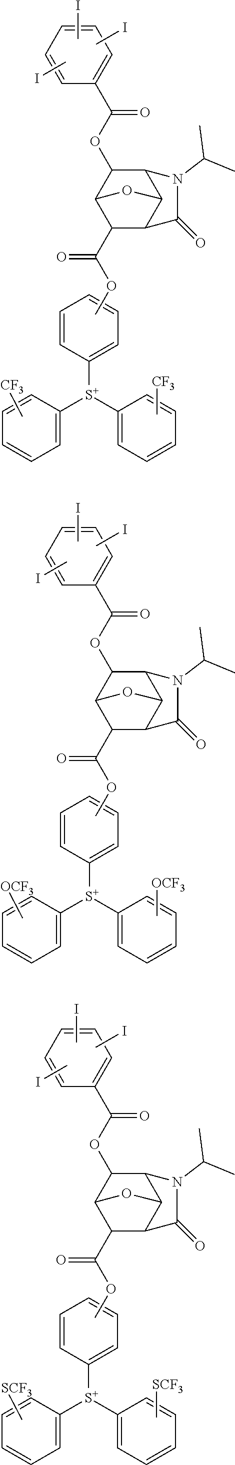

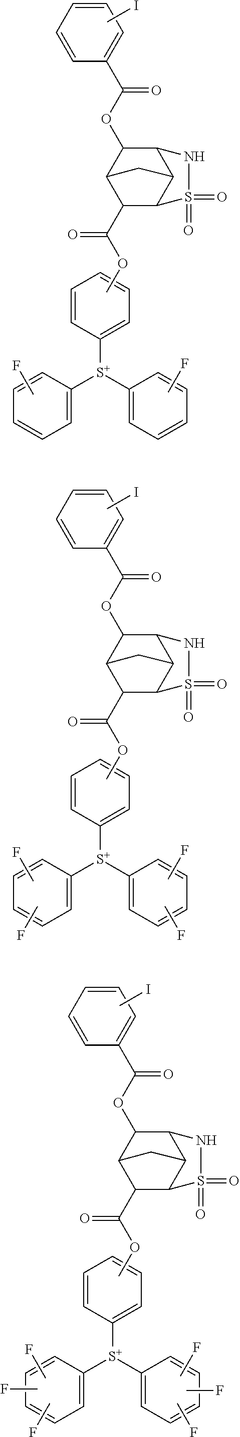

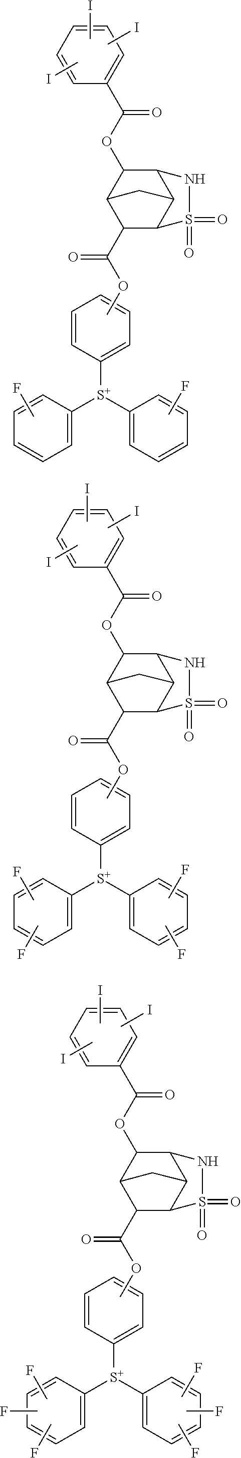

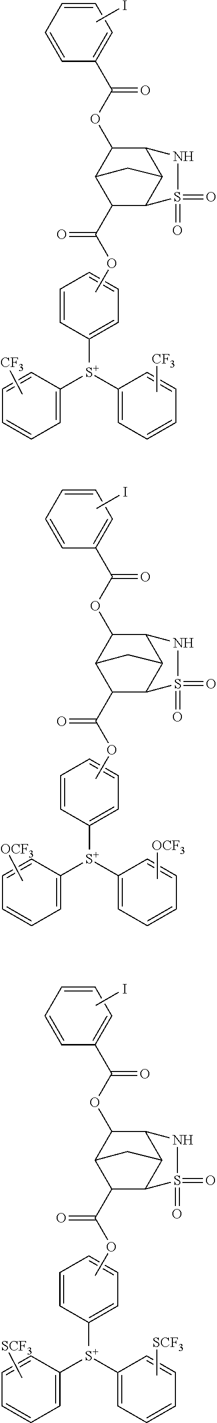

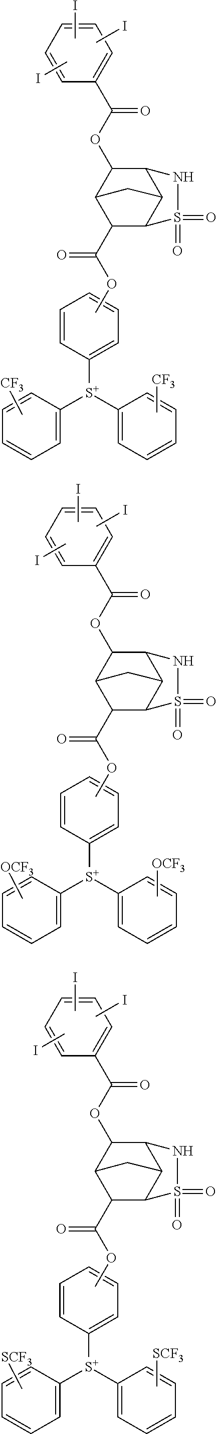

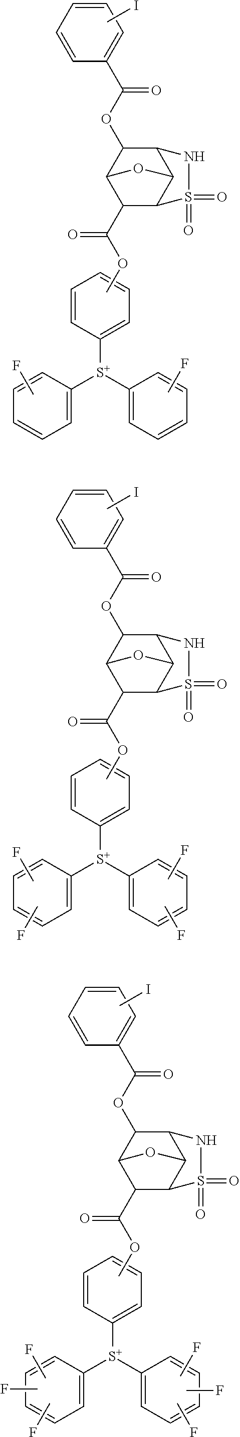

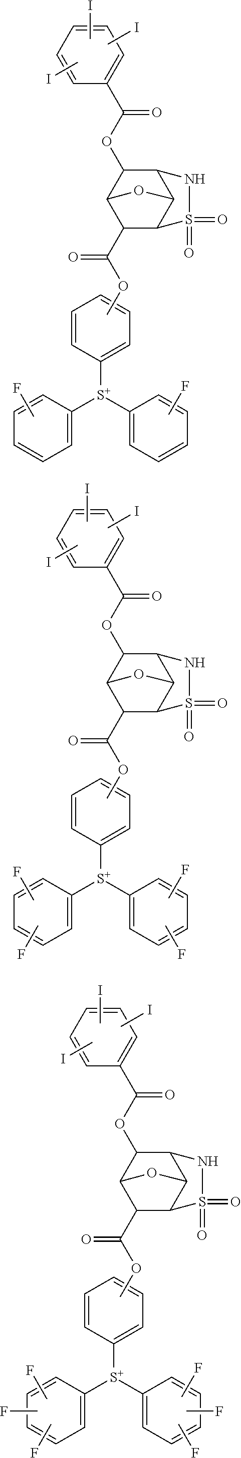

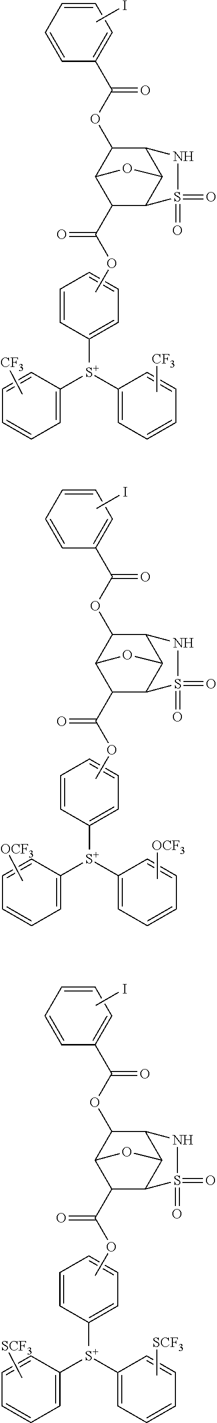

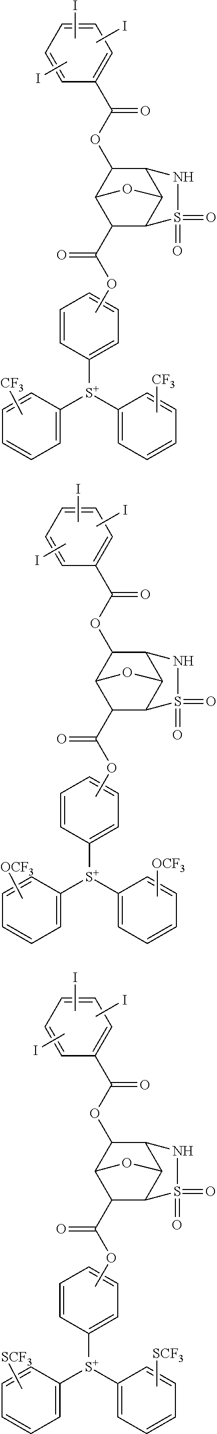

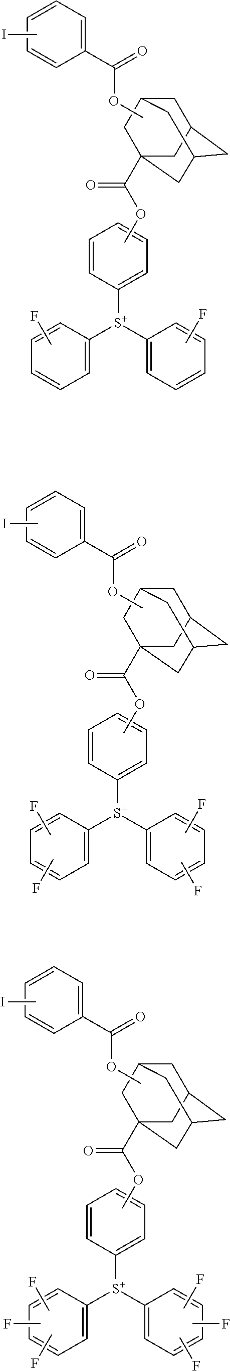

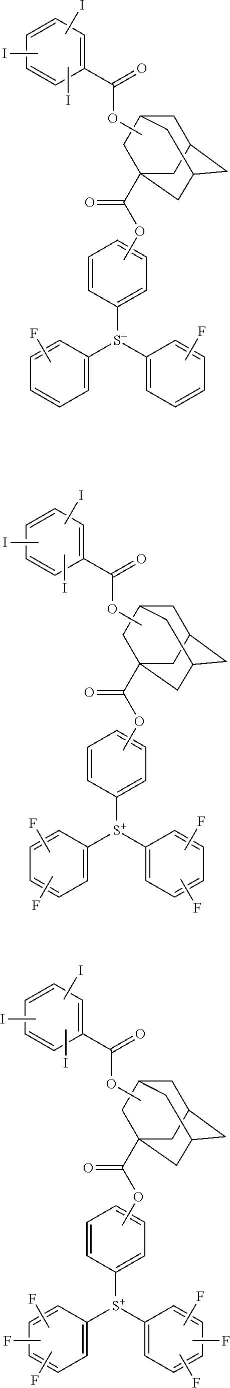

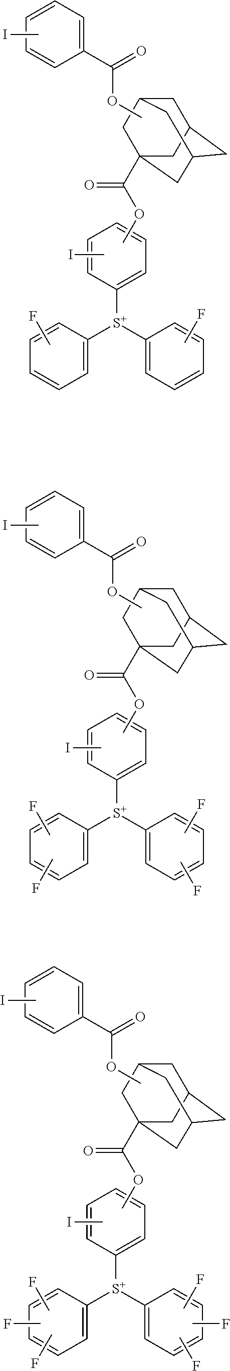

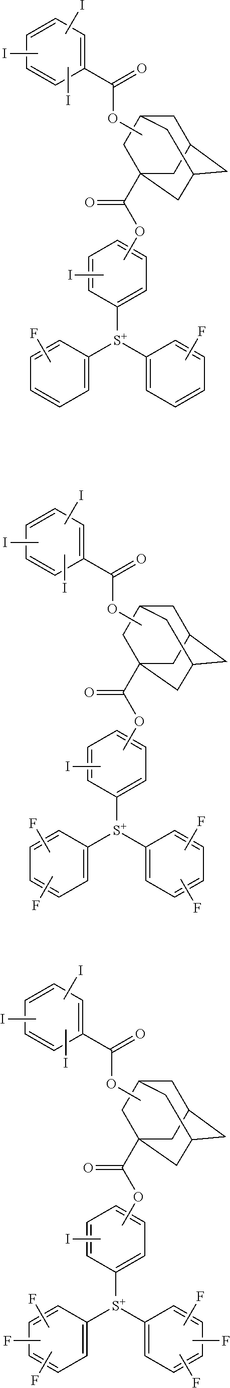

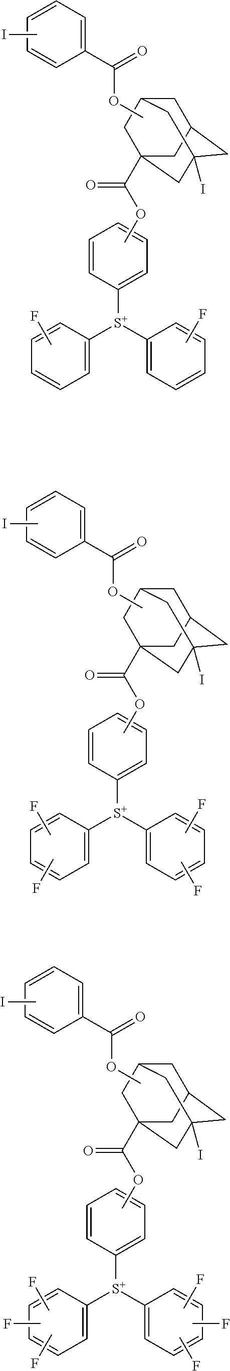

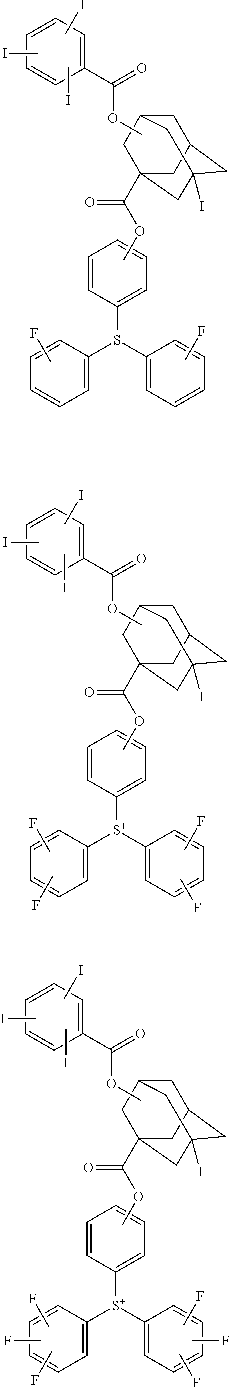

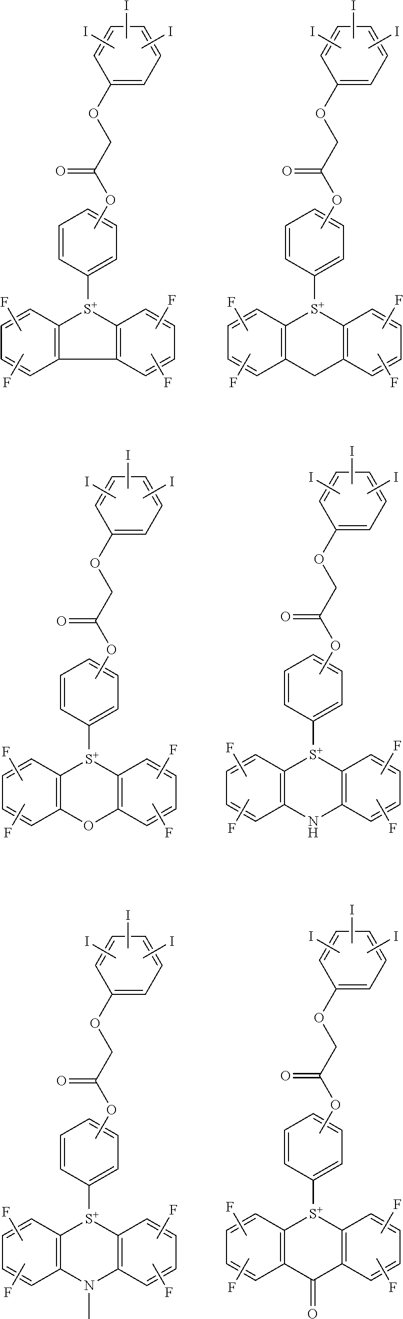

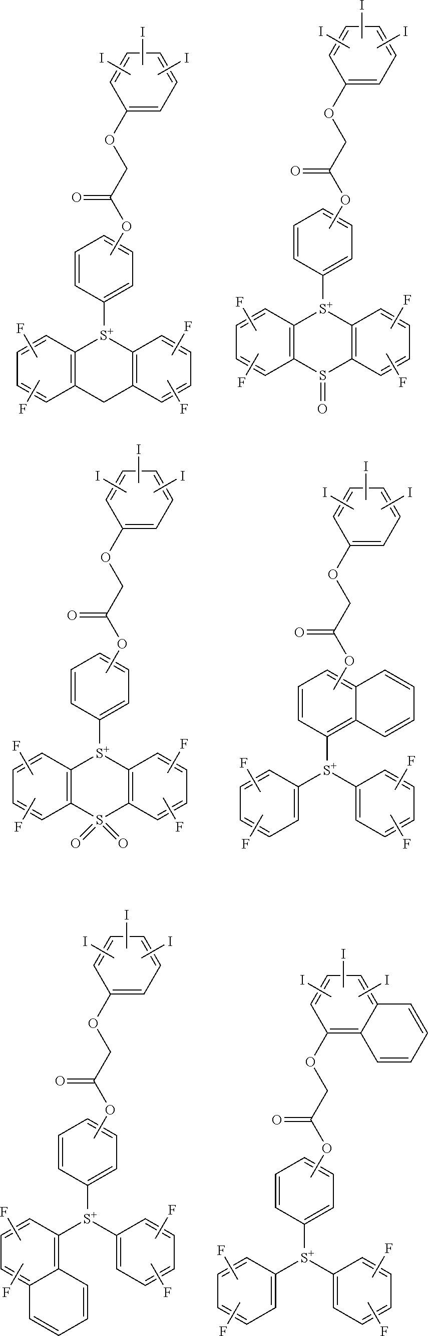

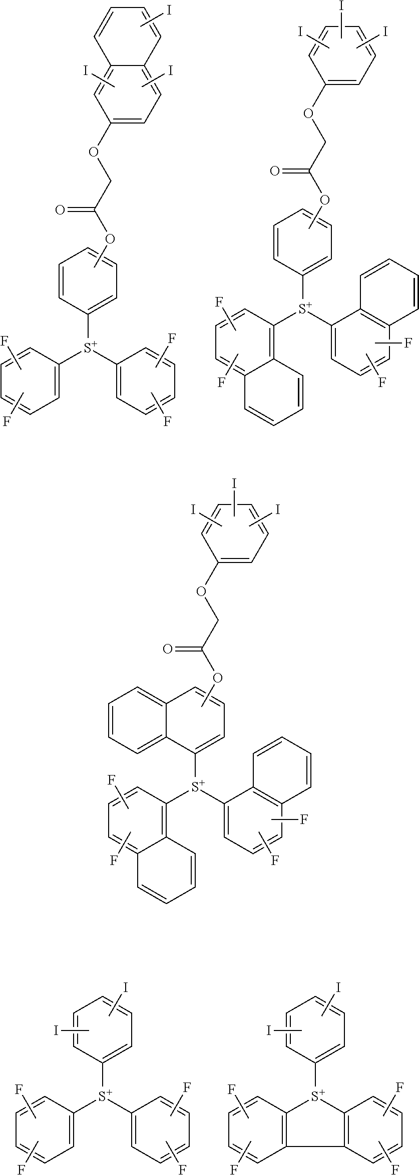

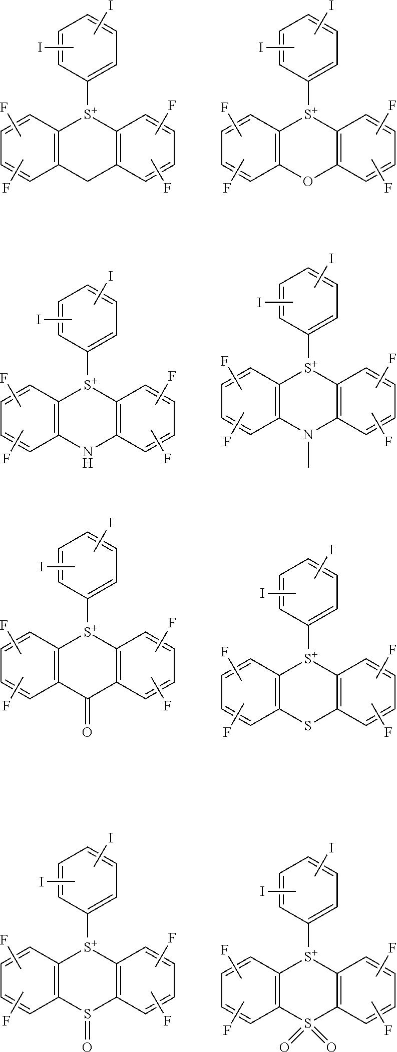

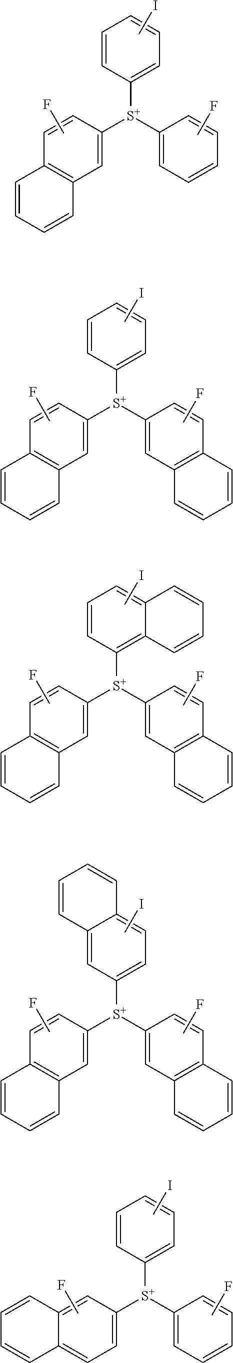

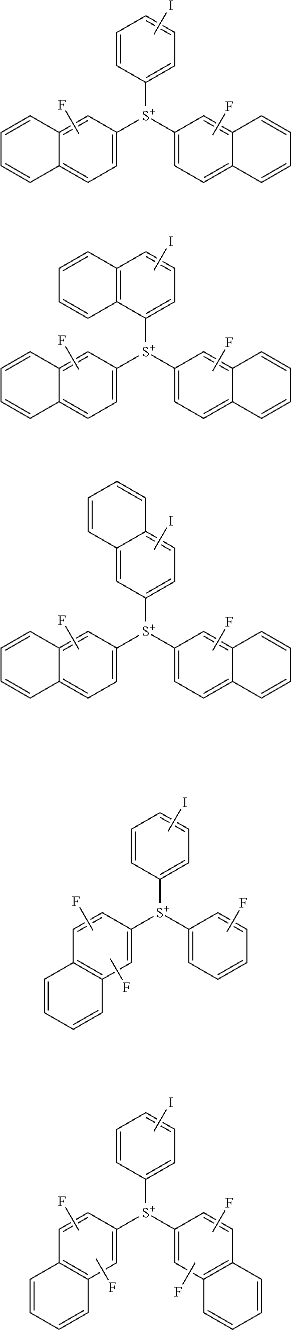

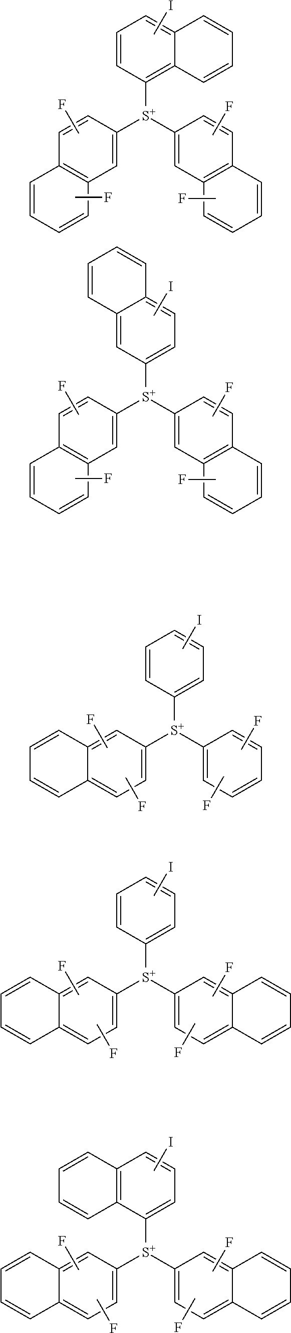

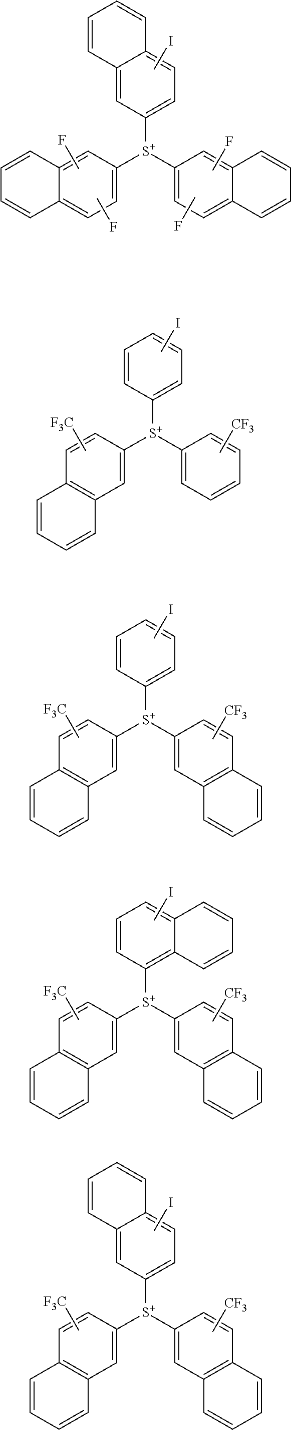

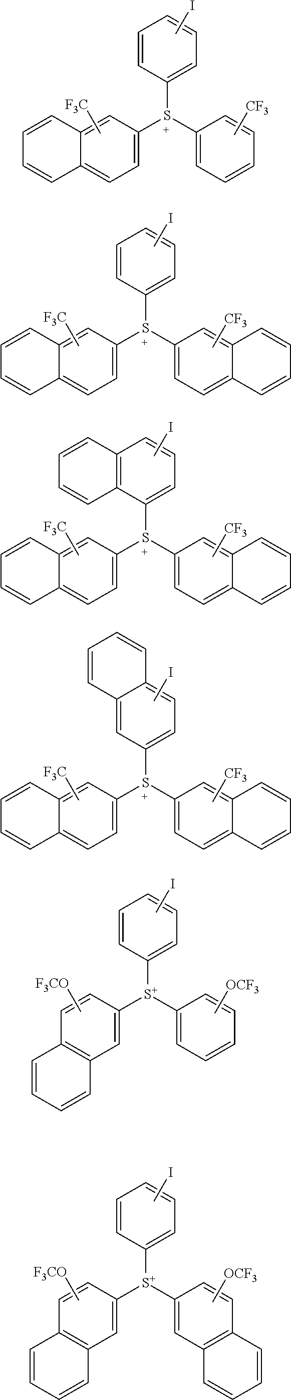

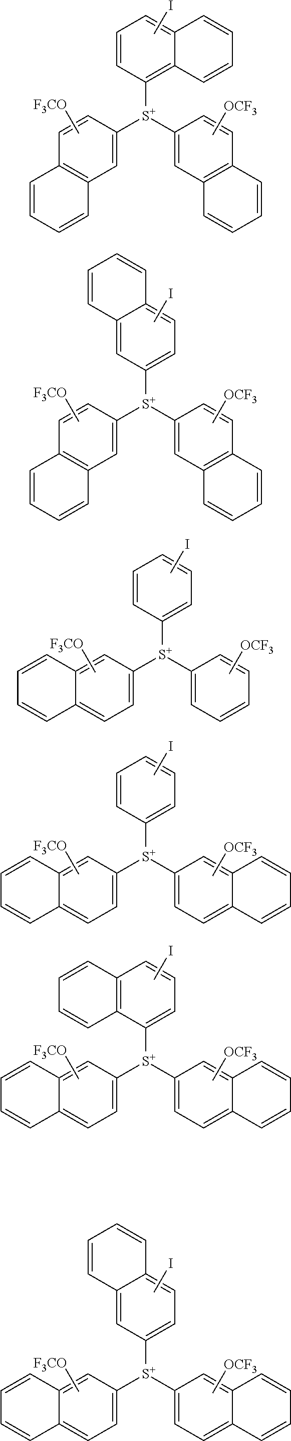

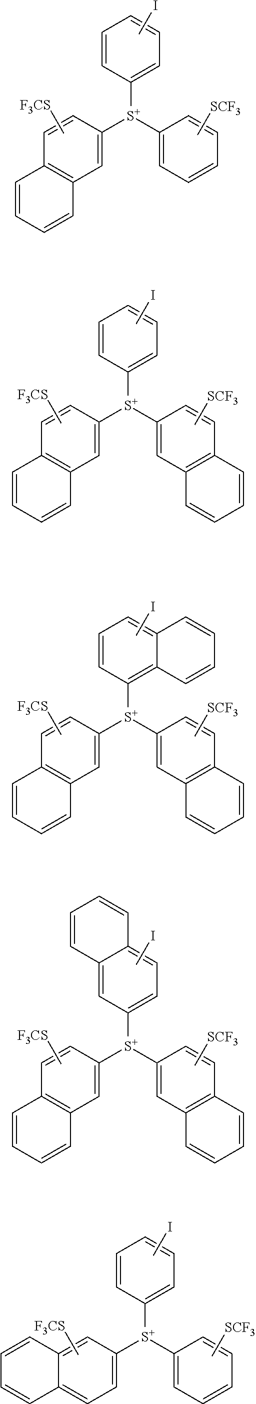

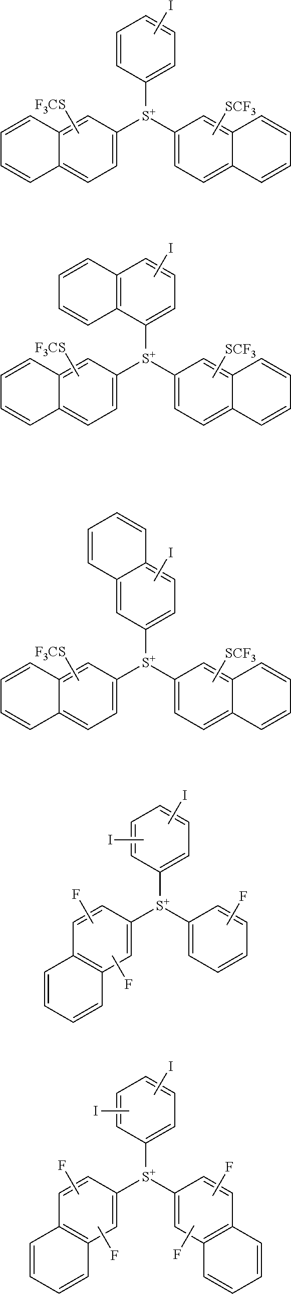

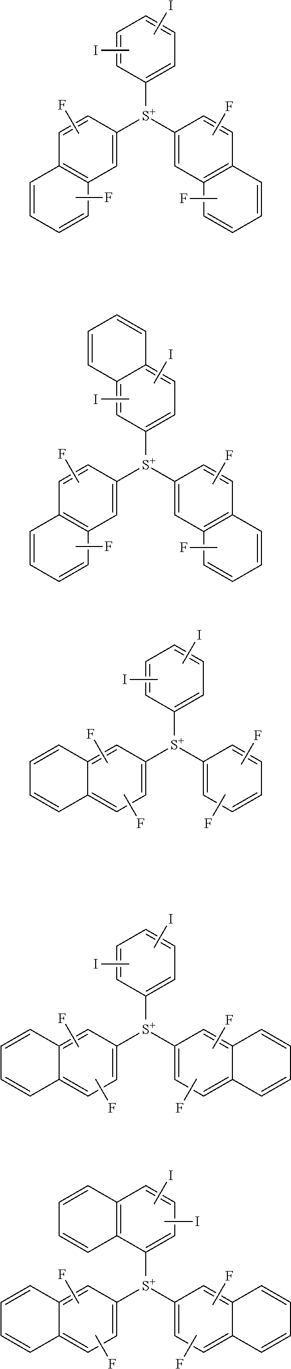

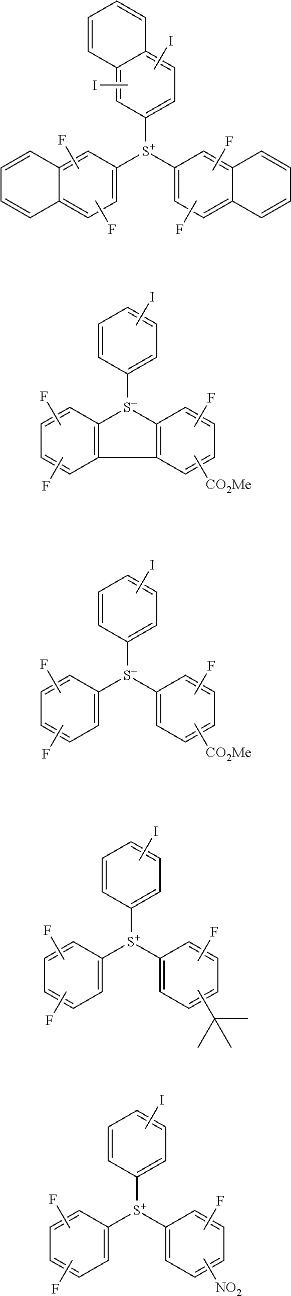

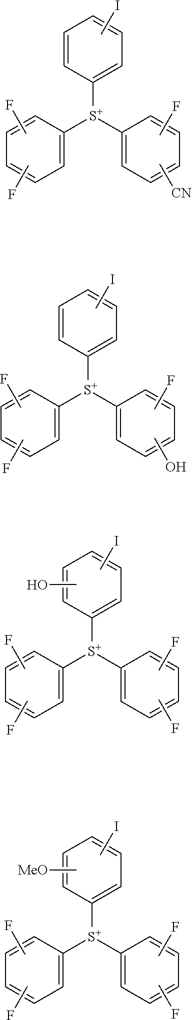

Specific examples of the onium salt of the invention include arbitrary combinations of anions with cations, both as exemplified above.

The onium salt of the invention can be synthesized, for example, by subjecting a hydrochloride or a carbonate having an onium cation to ion exchange with a corresponding benzoic acid derivative.

When the onium salt of the invention and an onium salt of a type that generates a strong acid such as a sulfonic acid which is fluorinated at α-position, an imidic acid, or a methidic acid (hereinafter, these are collectively defined as a strong acid) are allowed to coexist, a corresponding carboxylic acid and a strong acid are generated by light irradiation. On the other hand, the onium salt that is not decomposed is present in a large amount in a portion where the exposure dose is small. The strong acid functions as a catalyst for causing a deprotection reaction of a base resin, but hardly causes a deprotection reaction in the case of the onium salt of the invention. The strong acid undergoes ion exchange with the remaining sulfonium carboxylate to become an onium salt of the strong acid, and a carboxylic acid is released instead. In other words, by ion exchange, the strong acid is neutralized by the onium carboxylate. That is, the onium salt of the invention functions as a quencher. In this onium salt type quencher, LWR of resist patterns generally tends to be smaller than that of a quencher using an amine compound.

The salt exchange between the strong acid and the onium carboxylate is repeated countless times. The place where the strong acid is generated at the end of exposure is different from the place where the onium salt of a type that generates a strong acid is present at the beginning. It is presumed that the cycle of generation of an acid by light and salt exchange is repeated many times to average the generation points of the acid, thereby decreasing the LWR of resist patterns after development.

Further, the onium salt of the invention has an acid labile group and a —SF₅ group adjacent thereto in an aromatic ring in an anion as a structural feature in addition to the function as a quencher. The acid labile group in the exposed portion causes a deprotection reaction by the generated acid, and an aromatic hydroxy group is generated. As a result, the contrast between the exposed portion and the unexposed portion is improved. In addition, the adjacent-SFs group improves the resist solvent solubility of the sulfonium salt itself, and also improves the acidity of the aromatic hydroxy group generated in the exposed portion due to its strong electron withdrawing property. When the resist film is developed with an alkaline developer after exposure, the affinity between the generated aromatic hydroxy group and the alkaline developer is improved, so that the exposed portion is effectively removed by the developer. Further, it is considered that the aromatic hydroxy group adjacent to the —SF₅ group has an effect of reducing swelling caused by the alkaline developer without drawing the alkaline developer to the unexposed portion as compared with the carboxyl group due to the effect of water repellency of a fluorine atom. As a result, the collapse of the resist pattern occurring in the unexposed portion is suppressed. Due to these synergistic effects, when the onium salt of the invention is used, the dissolution contrast is high, the LWR of line patterns and the CDU of hole patterns are improved, and pattern formation resistant to pattern collapse becomes possible.

### Chemically Amplified Resist Composition

The chemically amplified resist composition of the invention contains (A) a quencher formed of an onium salt having the formula (1). In the invention, the quencher is a material for forming a desired pattern by trapping an acid generated from a photoacid generator in a chemically amplified resist composition to prevent diffusion to an unexposed portion.

In the chemically amplified resist composition of the invention, the content of the quencher formed of the onium salt having the formula (1) as the component (A) is preferably 0.001 to 30 parts by weight, and more preferably 0.01 to 20 parts by weight per 80 parts by weight of the base polymer described later. As the quencher (A), one type may be used alone or two or more types may be combined and used.

### (B) Base Polymer

The chemically amplified resist composition of the invention may contain a base polymer as a component (B). In the case of a positive resist composition, examples of the base polymer (B) include those containing a repeat unit having the formula (a1) (hereinafter also referred to as repeat unit (a1)) or a repeat unit having the formula (a2) (hereinafter also referred to as repeat unit (a2)).

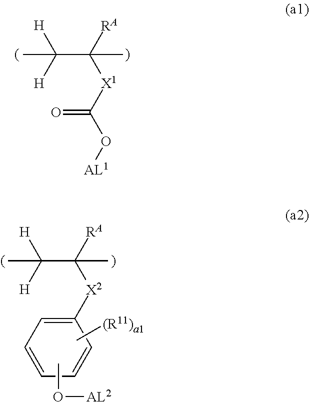

In the formulae (a1) and (a2), R^{A}'s are each independently a hydrogen atom, a fluorine atom, a methyl group, or a trifluoromethyl group.

In the formula (a1), X¹ is a single bond, a phenylene group, a naphthylene group, or *—C(═O)—O—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring, and * designates a point of attachment to the carbon atom in the backbone.

In the formula (a1), X² is a single bond or *—C(═O)—O—, * designates a point of attachment to the carbon atom in the backbone. R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, and a1 is 0, 1, 2, 3, or 4, preferably 0 or 1.

In the formulae (a1) and (a2), AL¹ and AL² are each independently an acid labile group. Specific examples of the acid labile group include those described in JP-A 2013-80033 and JP-A 2013-83821.

Typically, specific examples of the acid labile group include those having the formulae (AL-3) to (AL-5).

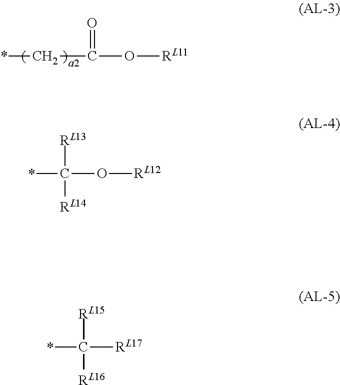

In the formulae, * designates a point of attachment.

In the formulae (AL-3) and (AL-4), R^{L11} and R^{L12} are each independently a C₁-C₄₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyloxy group.

In the formula (AL-3), a2 is an integer of 0 to 10, and is preferably 1, 2, 3, 4, or 5.

In the formula (AL-4), R^{L13} and R^{L14} are each independently a hydrogen atom, a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyloxy group. Any two of R^{L12}, R^{L13}, and R^{L14} may bond together to form a C₃-C₂₀ ring with the carbon atom or the carbon atom and the oxygen atom to which they are attached. The ring is preferably a C₄-C₁₆ ring, and particularly preferably an alicyclic ring.

In the formula (AL-5), R^{L15}, R^{L16}, and R^{L17} are each independently a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyloxy group. Any two of R^{L15}, R^{L16}, and R^{L17} may bond together to form a C₃-C₂₀ ring with the carbon atom to which they are attached. The ring is preferably a C₄-C₁₆ ring, and particularly preferably an alicyclic ring.

Specific examples of the repeat unit (a1) are shown below, but not limited thereto. In the following formulae. R^{A} and AL′ are as defined above.

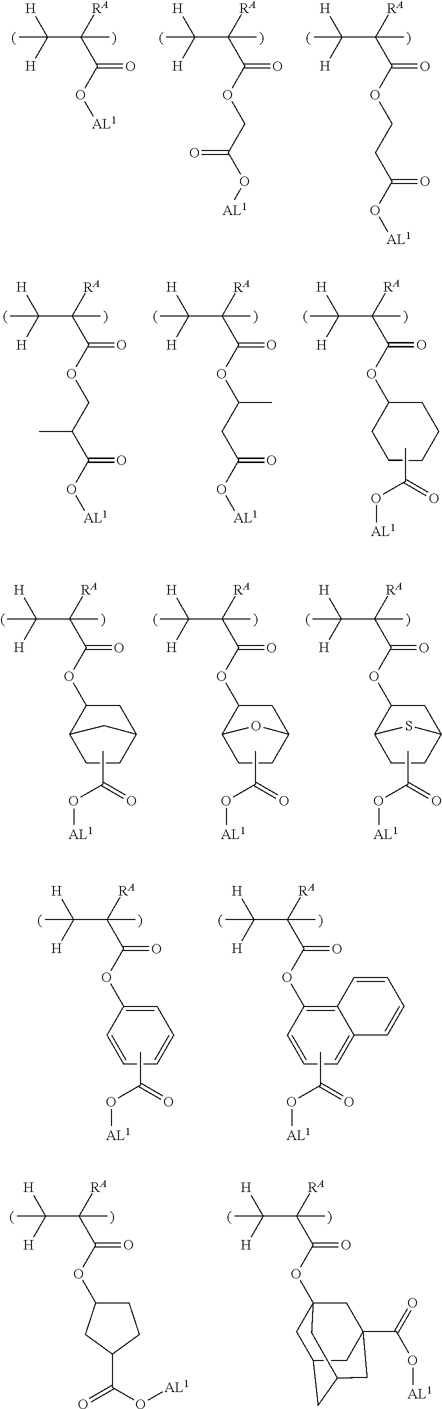

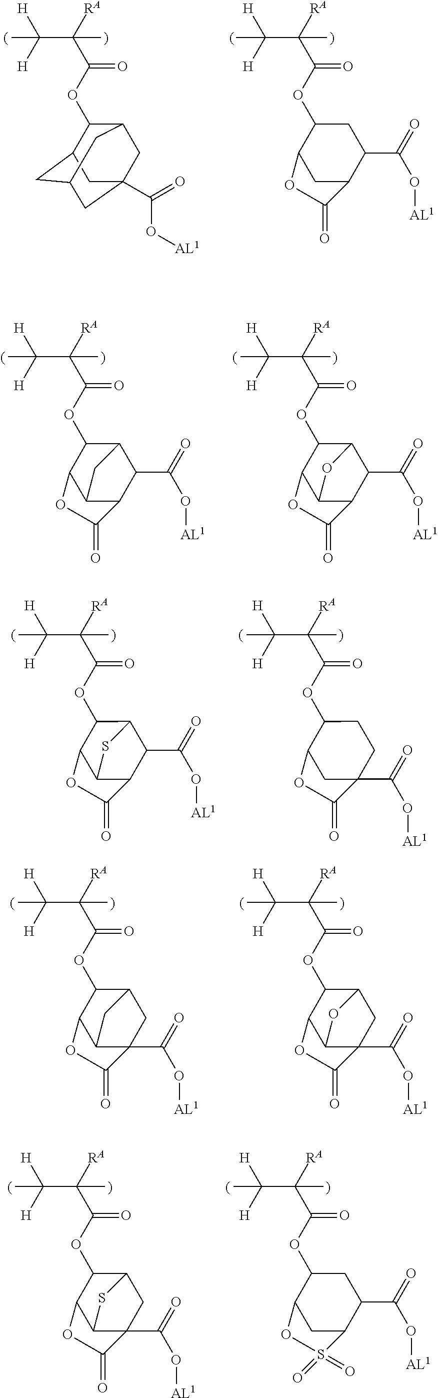

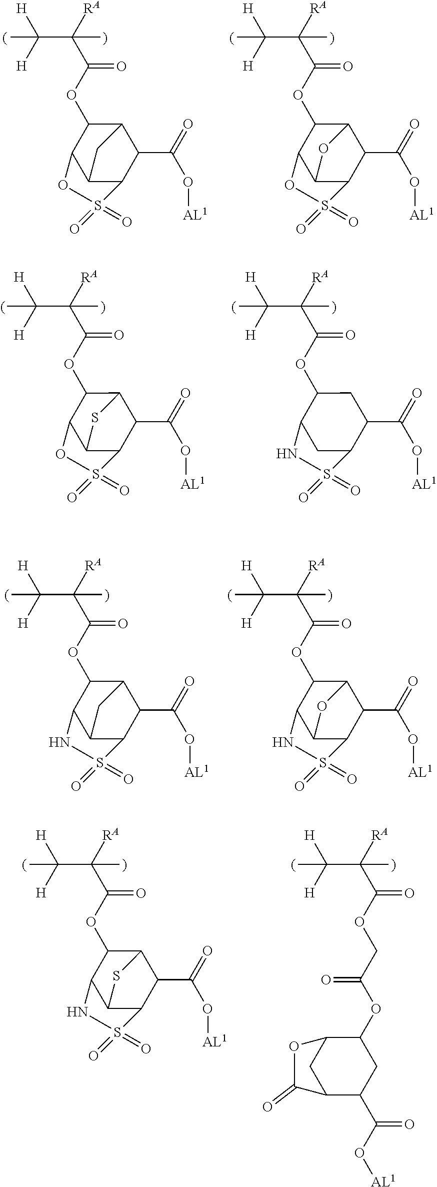

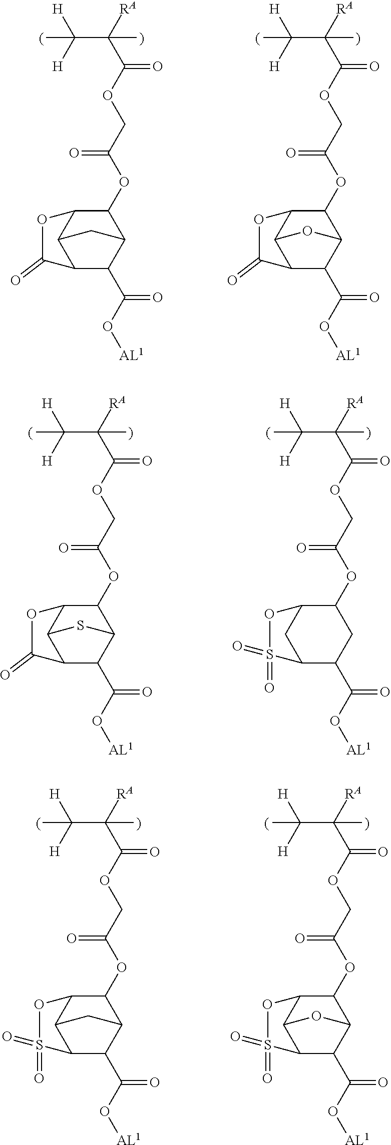

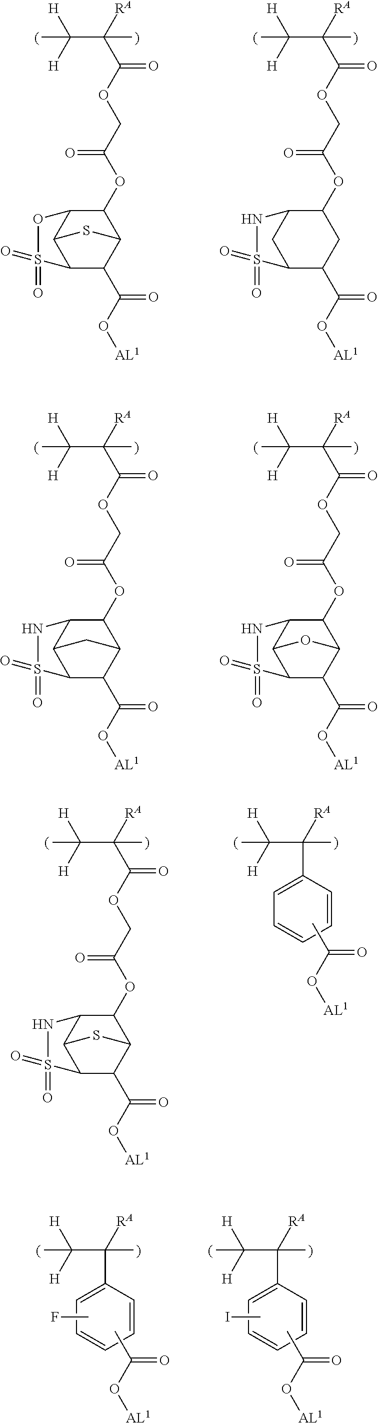

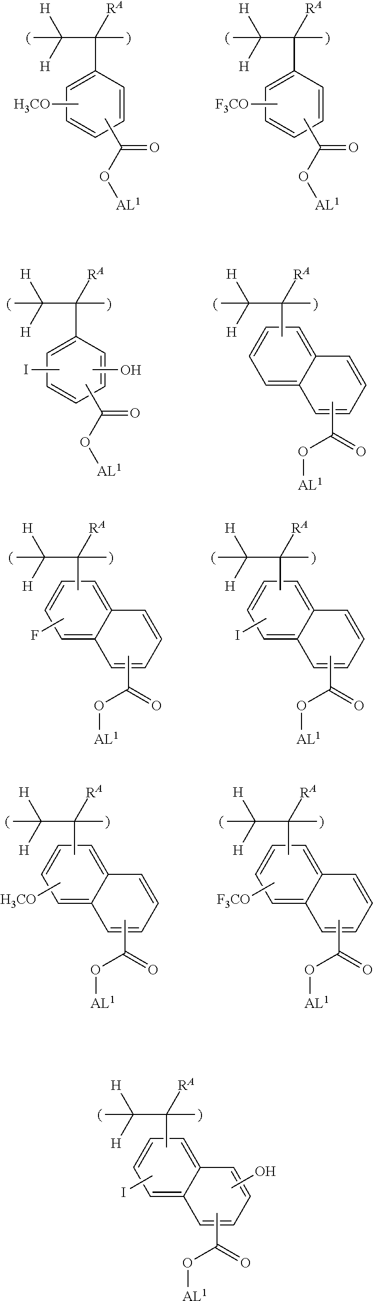

Specific examples of the repeat unit (a2) are shown below, but not limited thereto. In the following formulae. R^{A} and AL? are as defined above.

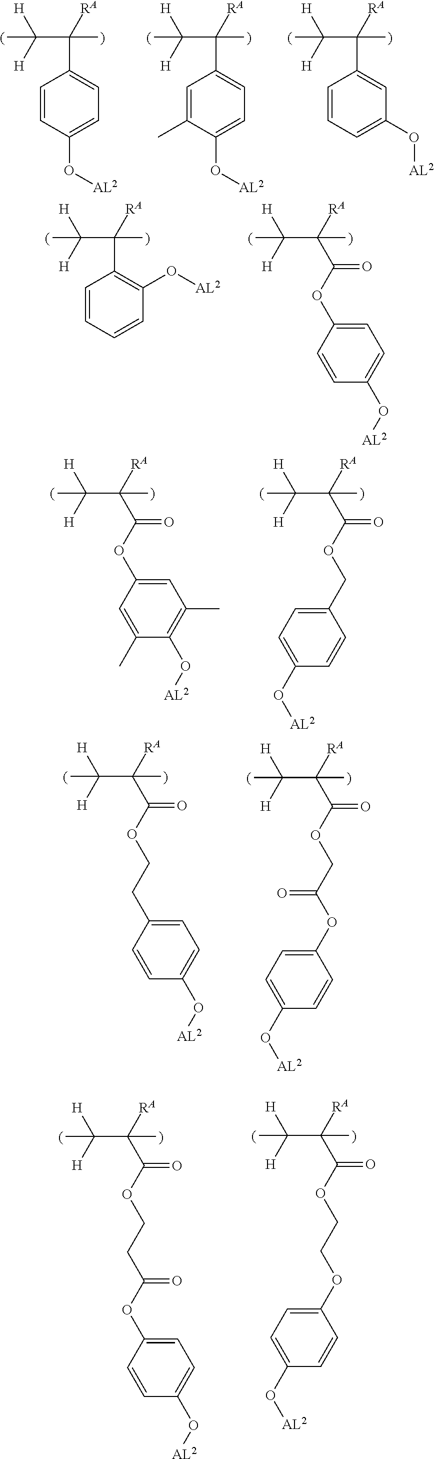

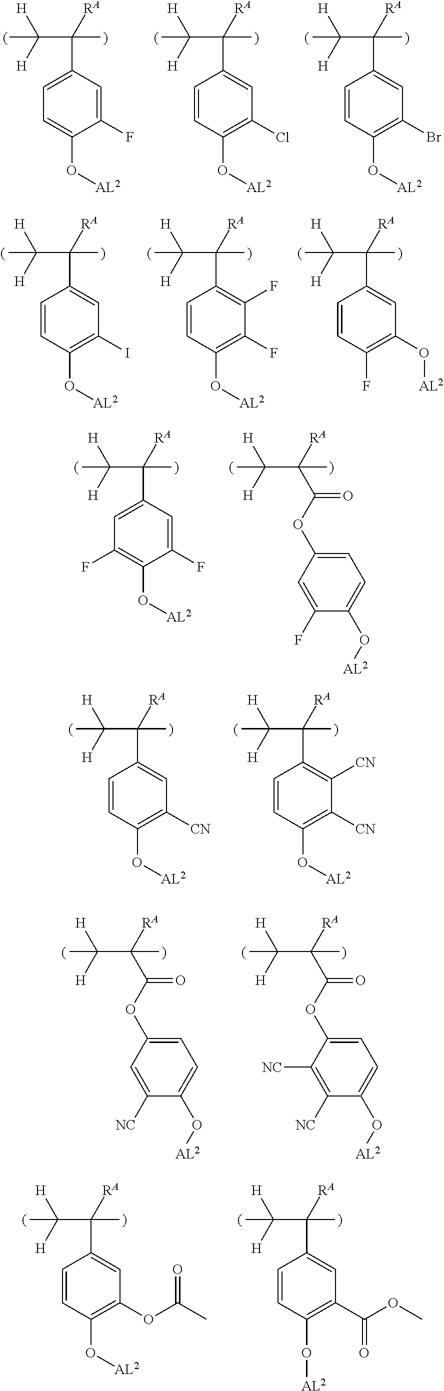

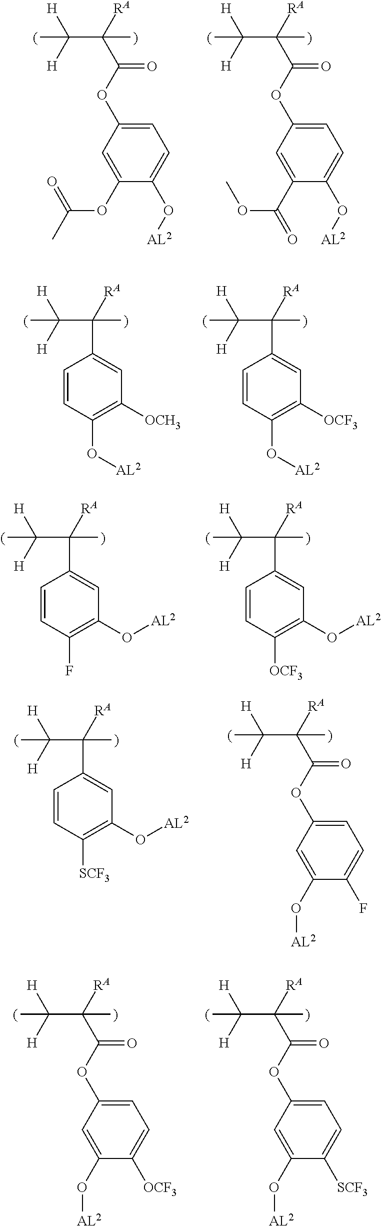

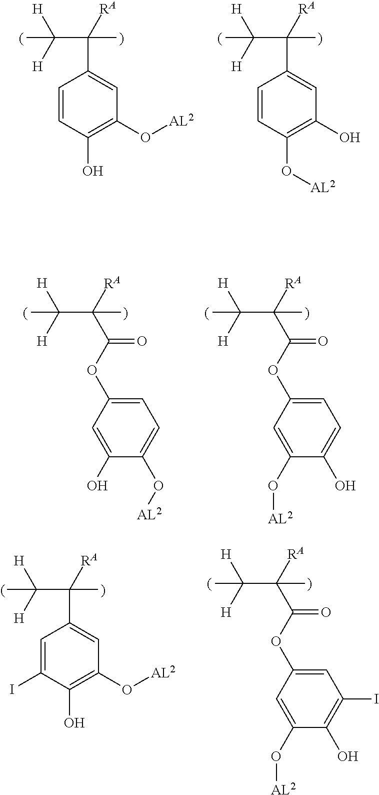

When the chemically amplified resist composition of the invention is of positive tone, the base polymer may contain a repeat unit having the formula (a3) (hereinafter also referred to as repeat unit (a3)).

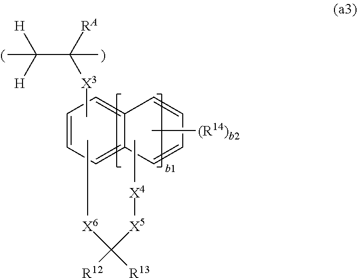

In the formula (a3), b1 is 0 or 1. The relevant structure is a benzene ring when b1 is 0 and a naphthalene ring when b1 is 1, and from the aspect of solvent solubility, a benzene ring when b1 is 0 is preferred, b2 is 0, 1, 2, or 3 when b1 is 0, and is 0, 1, 2, 3, 4, or 5 when b1 is 1. From the aspect of raw material procurement, b2 is preferably 0, 1, 2, or 3, and more preferably 0, 1, or 2.

In the formula (a3), R^{A} is a hydrogen atom, a fluorine atom, a methyl group, or a trifluoromethyl group, Among them, a hydrogen atom and a methyl group are preferred, and a hydrogen atom is more preferred.

In the formula (a3), X³ is a single bond, *—C(═O)—O—, or *—C(═O)—NH—. * designates a point of attachment to the carbon atom in the backbone. Among them, a single bond and *—C(═O)—O—are preferred, and a single bond is more preferred.

In the formula (a3), X⁴ is a single bond, a C₁-C₄ aliphatic hydrocarbylene group, a carbonyl group, a sulfonyl group, or a group obtained by combining these groups. Among them, a single bond, a carbonyl group, or a sulfonyl group is preferred from the aspect of raw material procurement, and a single bond or a carbonyl group is more preferred from the aspect of a polar group generated after the reaction.

In the formula (a3), X⁵ and X⁶ are each independently an oxygen atom or a sulfur atom. X⁴ and X⁶ are attached to the adjacent carbon atoms on the aromatic rings. X⁵ and X⁶ may be identical or different, but X⁵ and X⁶ are both preferably an oxygen atom from the aspect of reactivity.

In the formula (a3), R¹² and R¹³ are each independently a hydrogen atom or a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group and a naphthyl group; C₇-C₂₀ aralkyl groups such as a benzyl group, a 1-phenylethyl group, and a 2-phenylethyl group; and a group obtained by combining these groups. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

R¹² and R¹³ may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. Some or all hydrogen atoms in the ring may be substituted with a group containing a heteroatom such as an oxygen atom, a sulfur atom, a nitrogen atom, or a halogen atom, and some —CH₂— in the ring may be replaced by a group containing a heteroatom such as an oxygen atom, a sulfur atom, or a nitrogen atom, so that the ring may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

In the formula (a3), R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A}) (R^{14B}). R^{14A} and R^{14B} are each independently a hydrogen atom or a C₁-C₆ hydrocarbyl group. The halogen atom is preferably a fluorine atom, a chlorine atom, a bromine atom, or an iodine atom, and more preferably a fluorine atom or an iodine atom.

The hydrocarbyl group and the hydrocarbyl moiety of the hydrocarbyloxy group, the hydrocarbyloxycarbonyl group, and the hydrocarbylthio group may be saturated or unsaturated and straight, branched, or cyclic. Specific examples thereof are as exemplified above for the hydrocarbyl group of R¹² and R¹³. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. When b2 is 2 or more, a plurality of R¹⁴'s may be identical or different.

When b2 is 2 or more, a plurality of R¹⁴'s may bond together to form a ring with the carbon atom on an aromatic ring to which they are attached. Specific examples of the ring formed at this time include a cyclopropane ring, a cyclobutane ring, a cyclopentane ring, a cyclohexane ring, a norbornane ring, and an adamantane ring. Some or all hydrogen atoms in the ring may be substituted with a group containing a heteroatom such as an oxygen atom, a sulfur atom, a nitrogen atom, or a halogen atom, and some —CH₂— in the ring may be replaced by a group containing a heteroatom such as an oxygen atom, a sulfur atom, or a nitrogen atom, so that the ring may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Specific examples of the repeat unit (a3) are shown below, but not limited thereto. In the following formulae, R^{A} is as defined above, and Me is a methyl group. The positions of attachment of substituents on the aromatic ring are interchangeable.

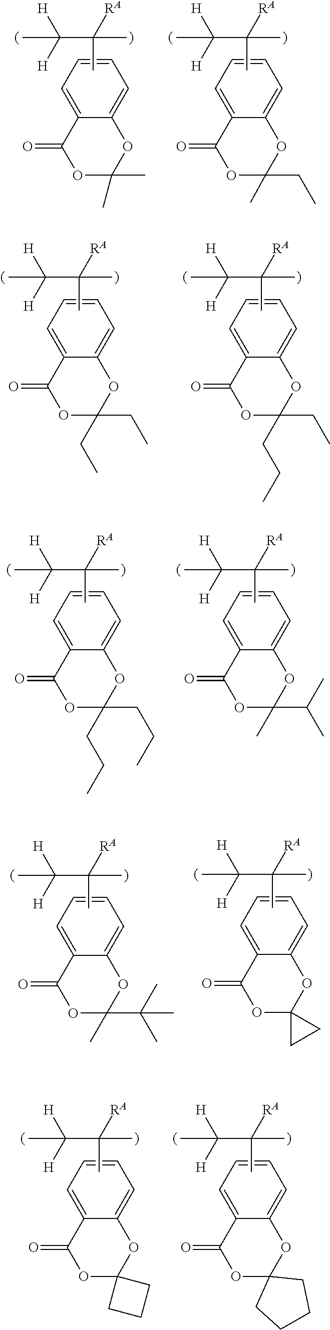

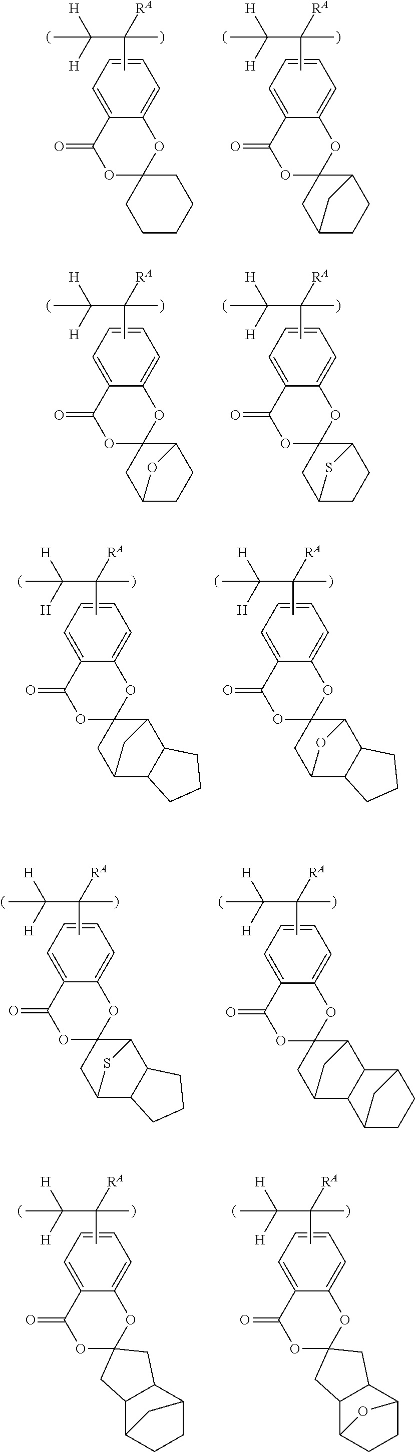

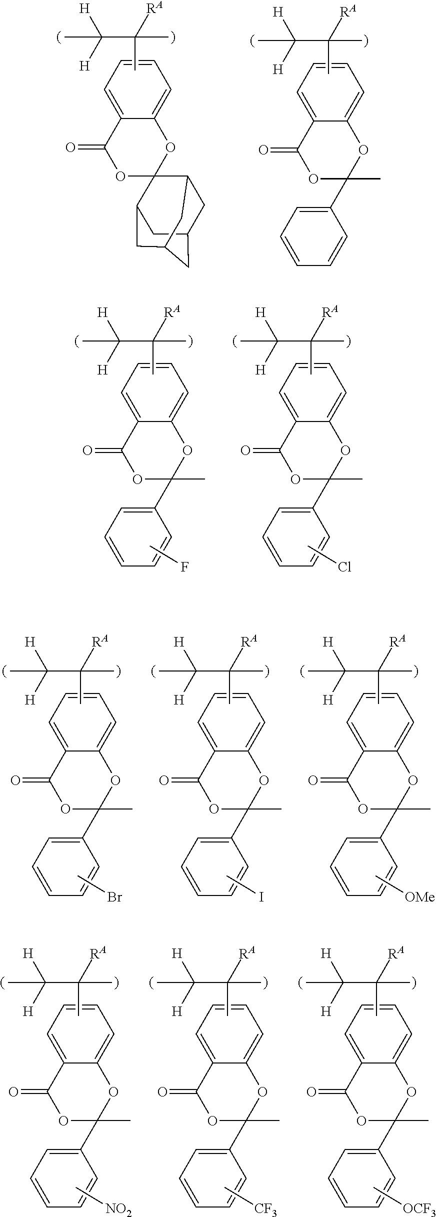

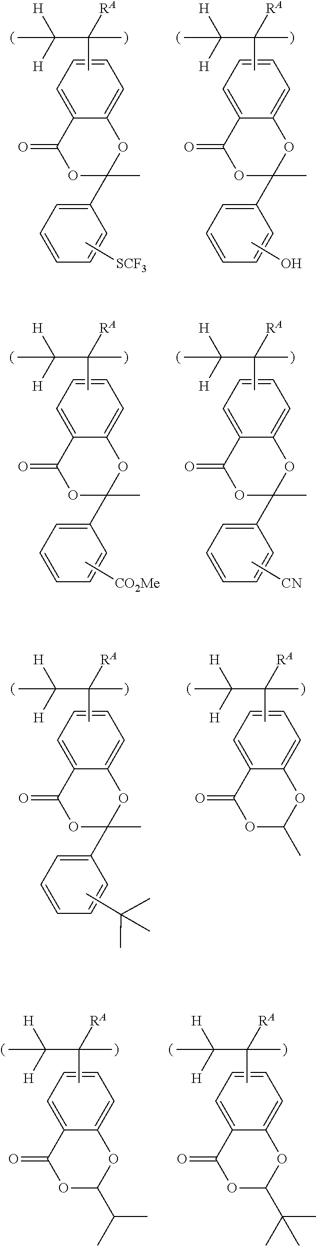

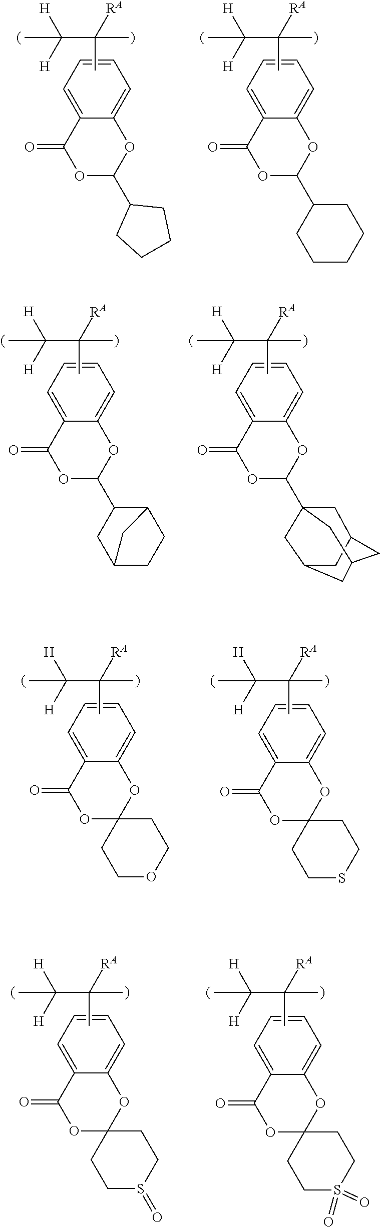

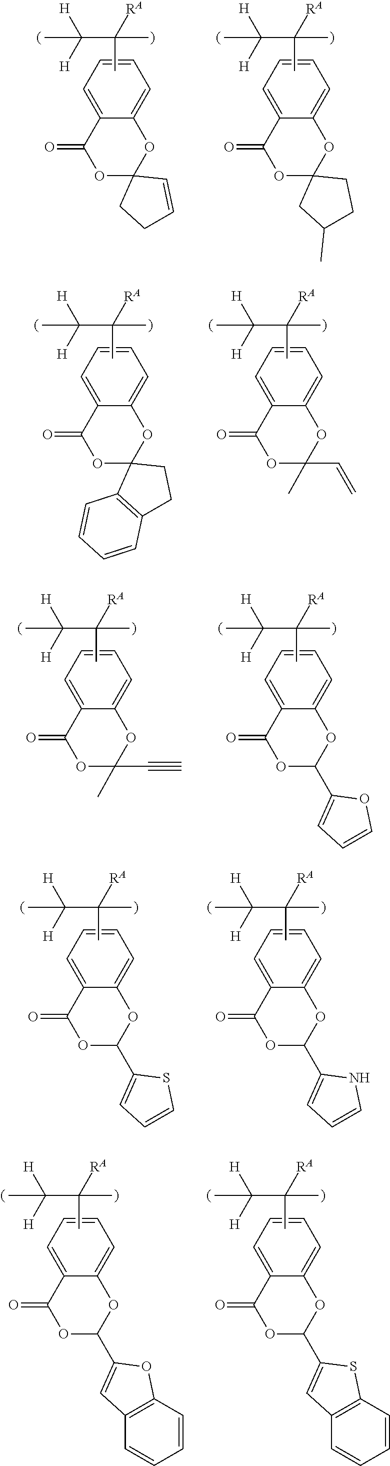

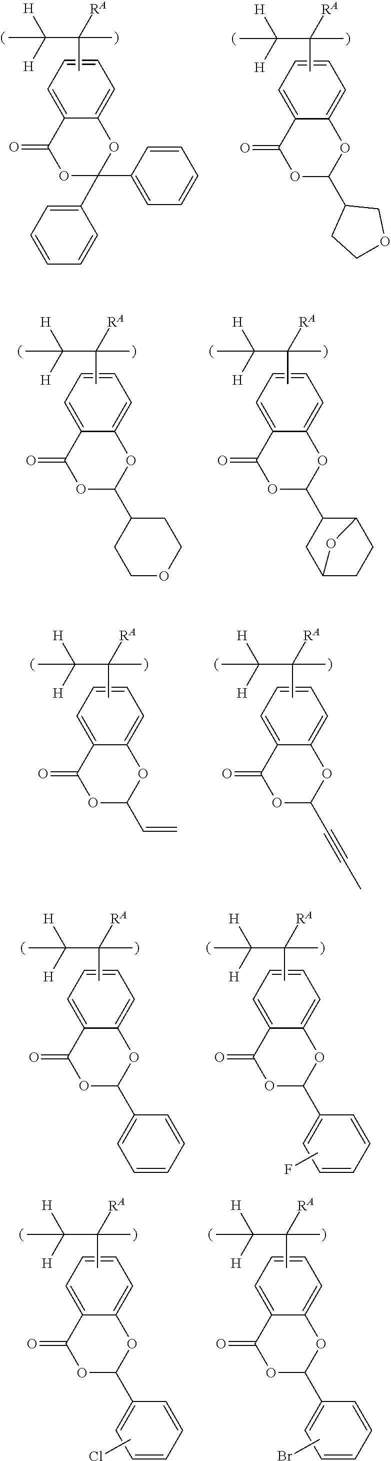

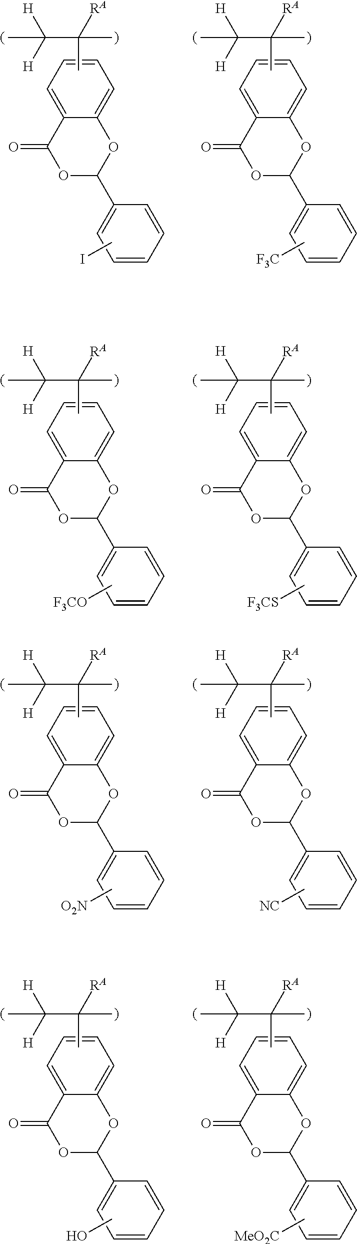

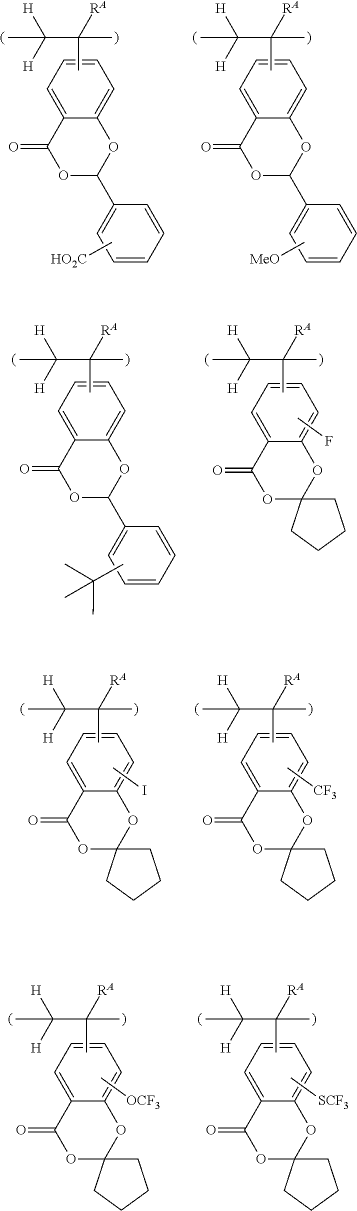

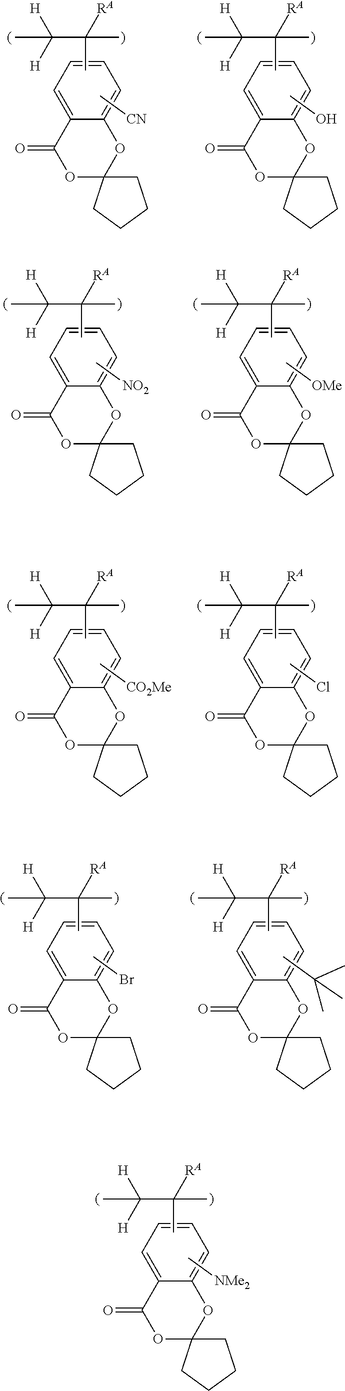

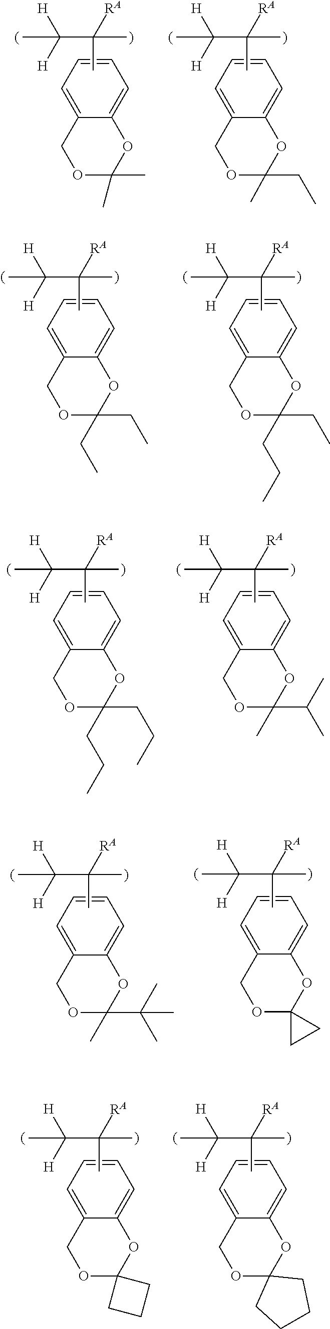

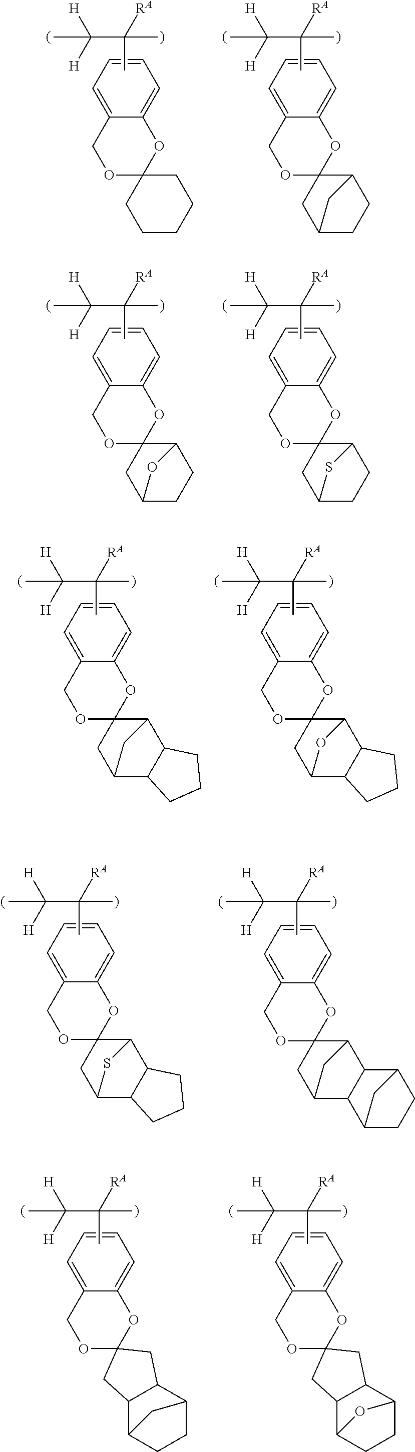

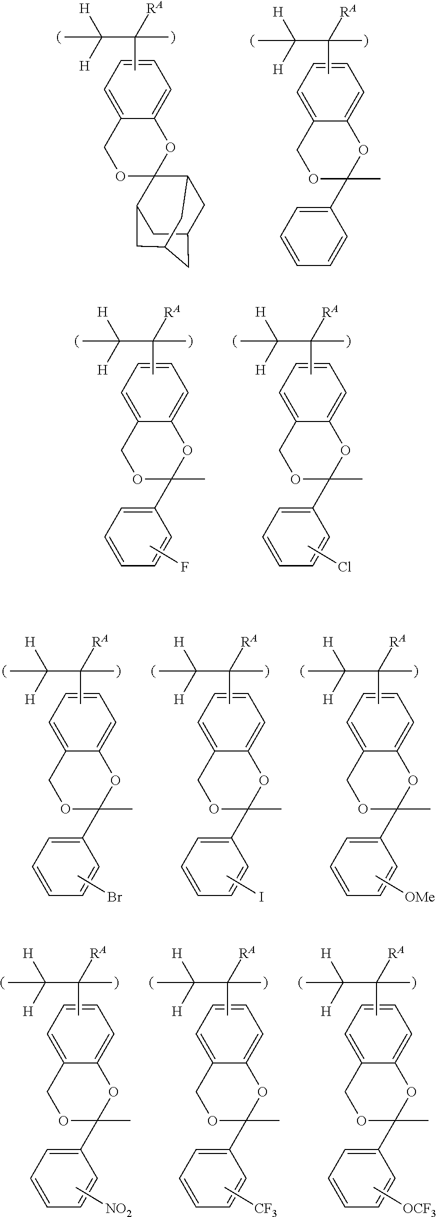

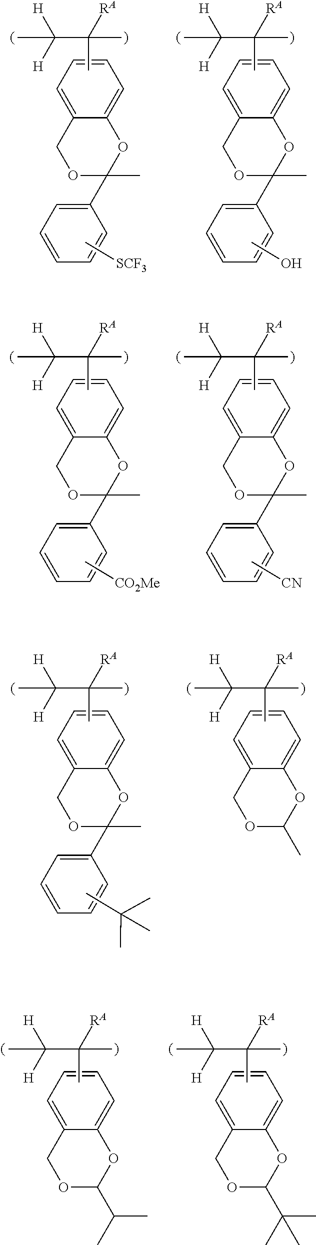

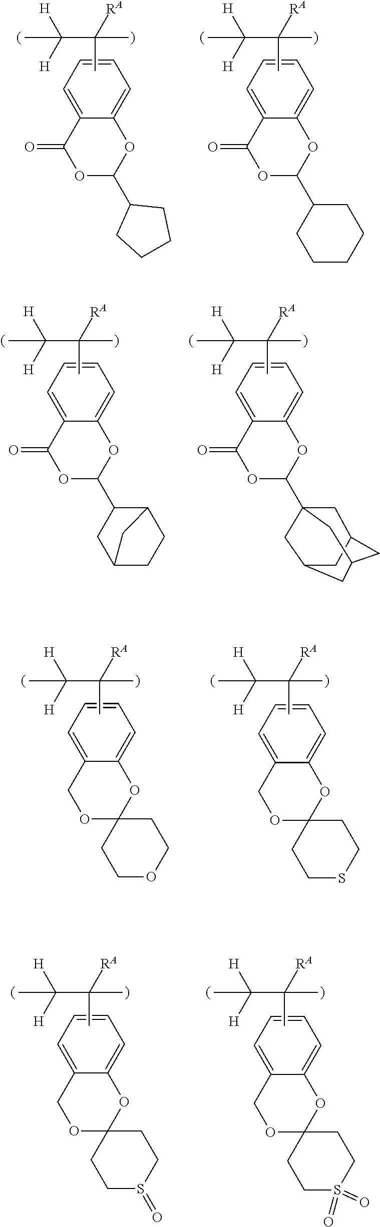

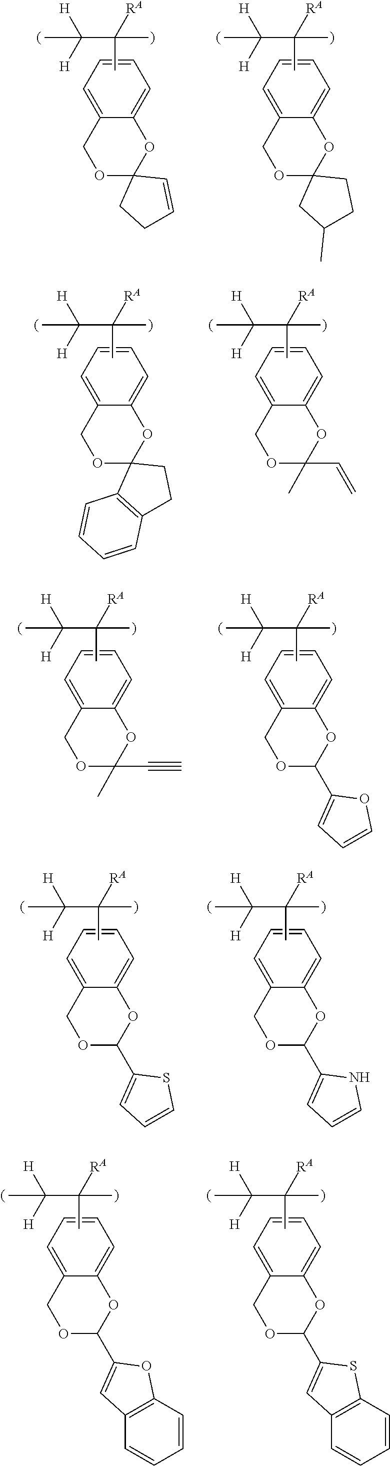

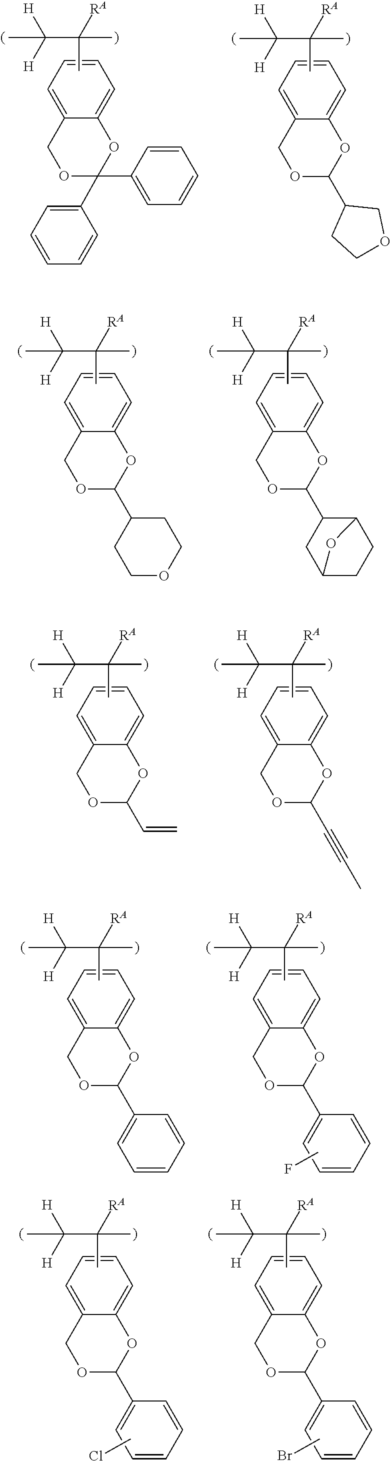

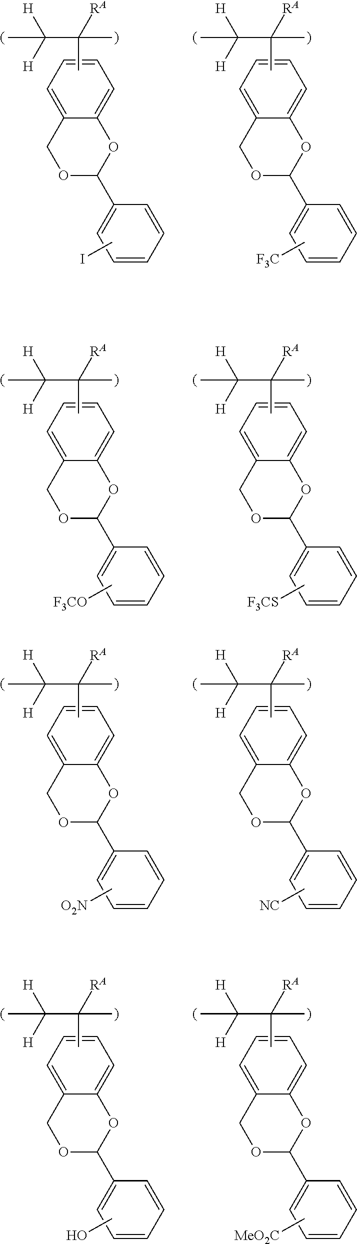

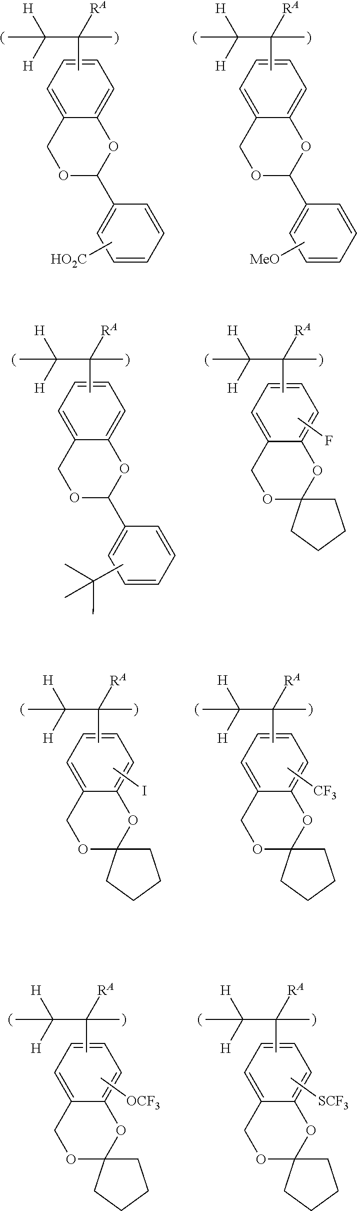

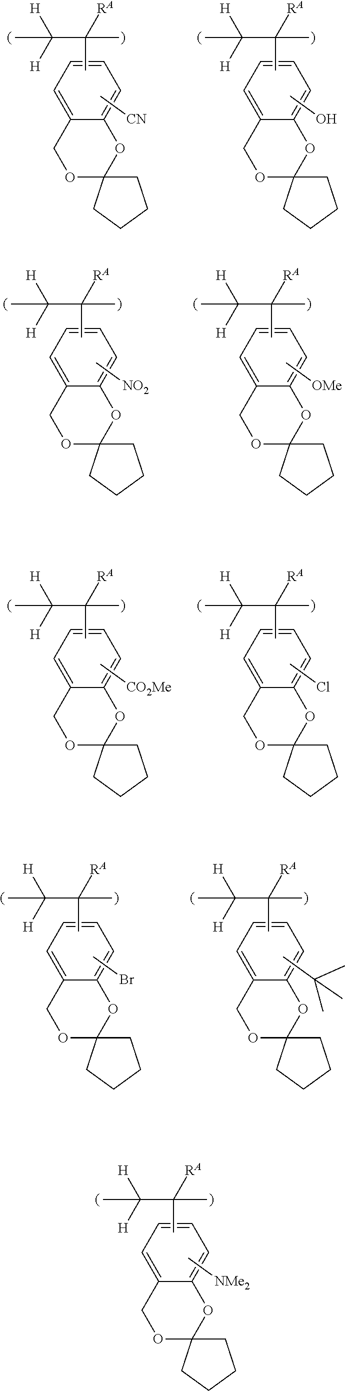

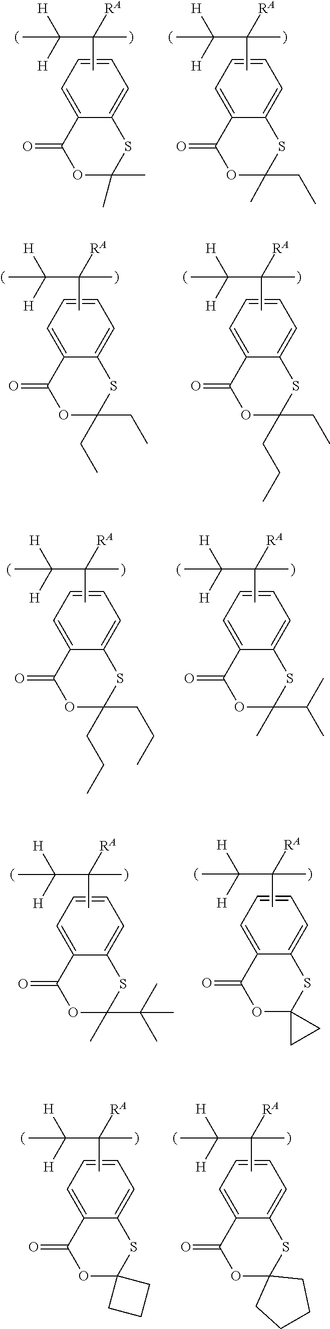

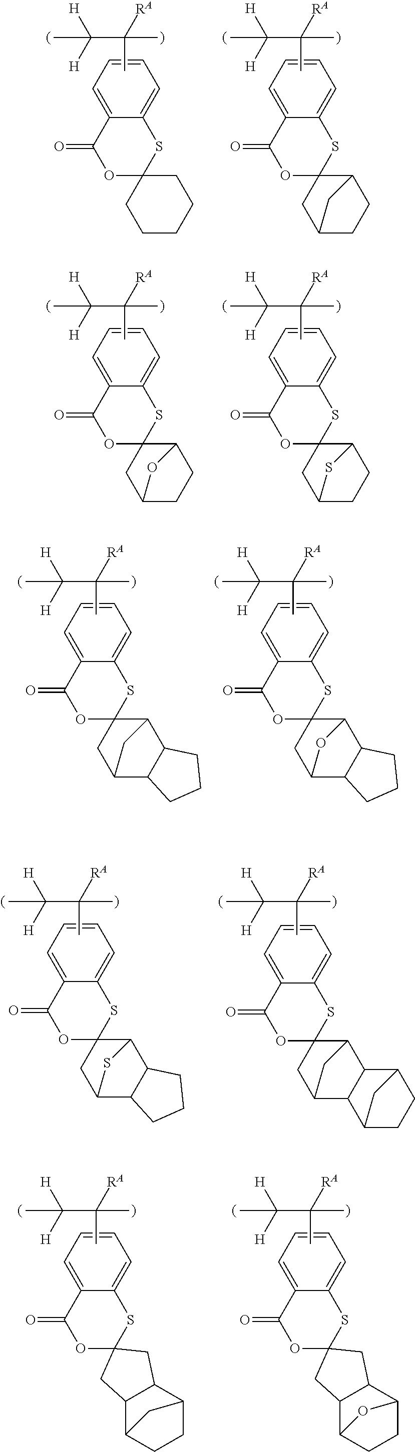

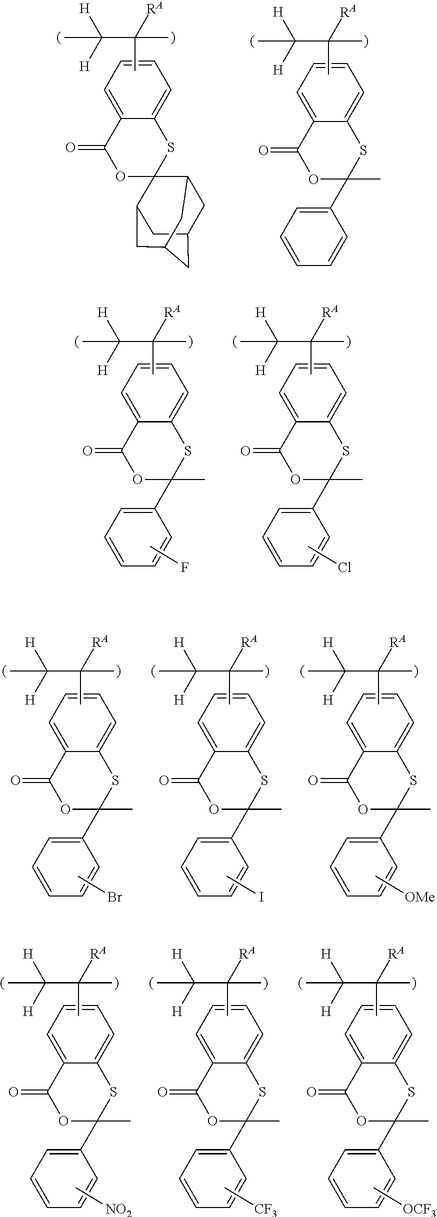

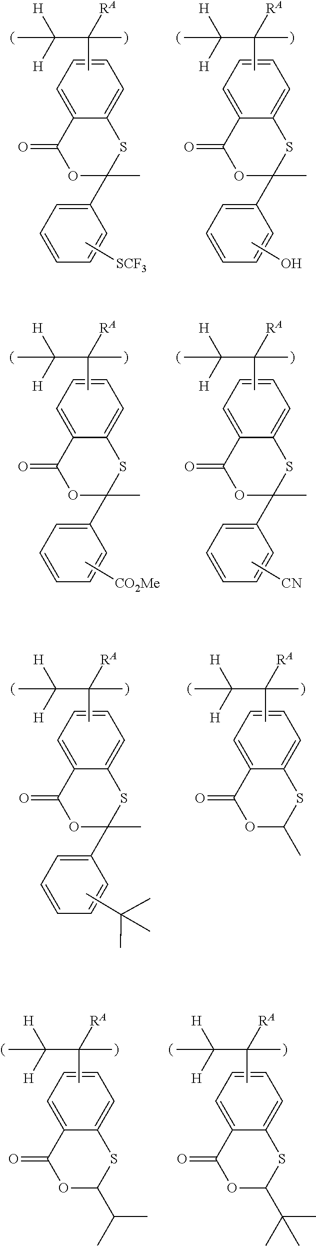

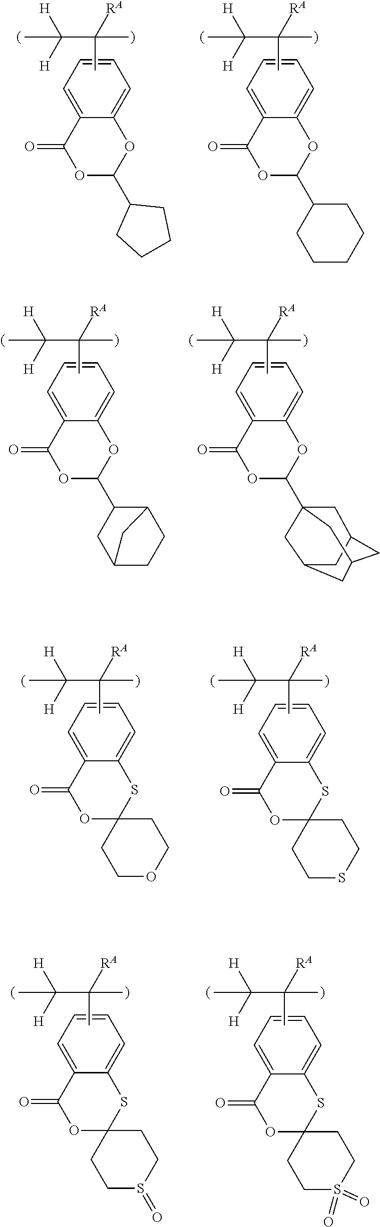

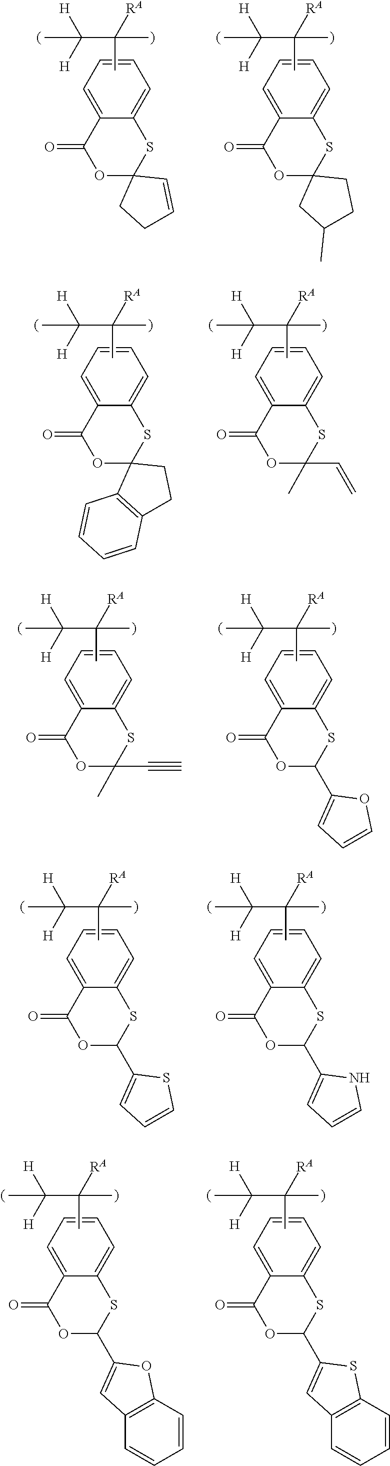

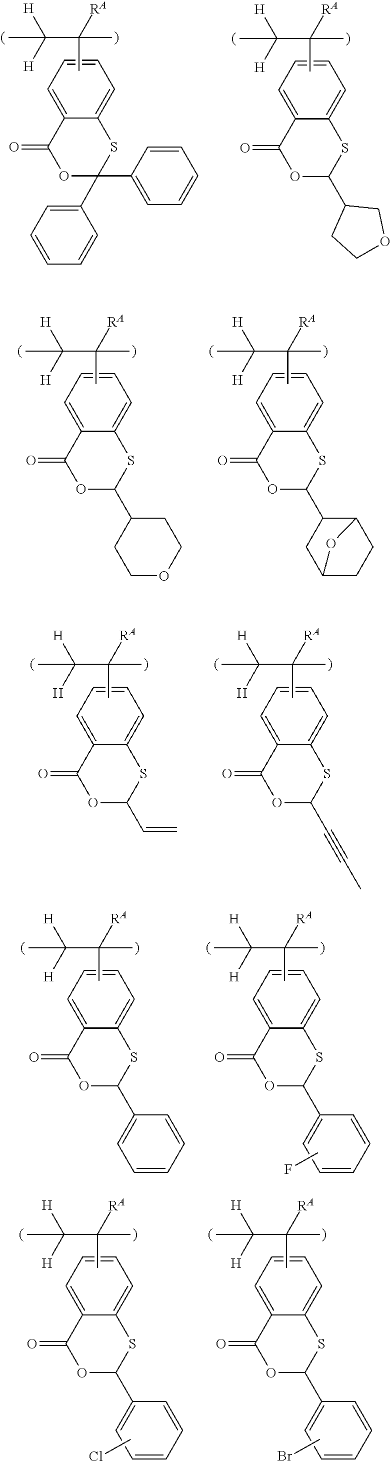

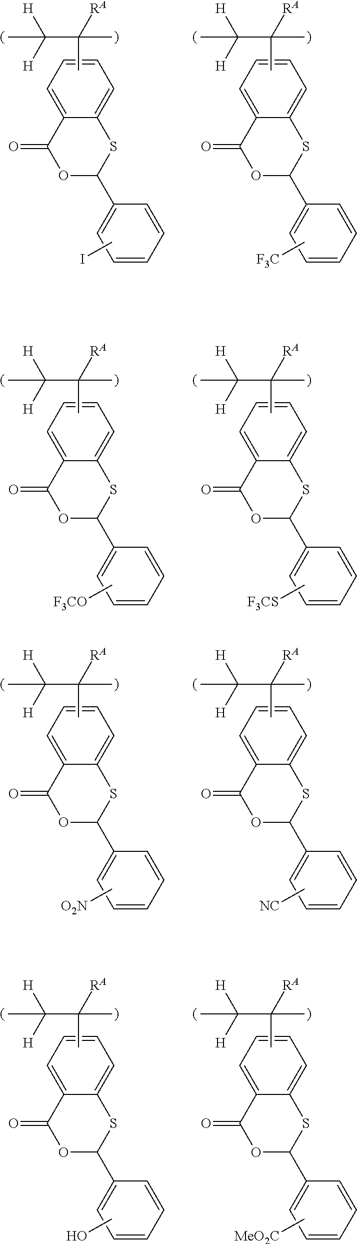

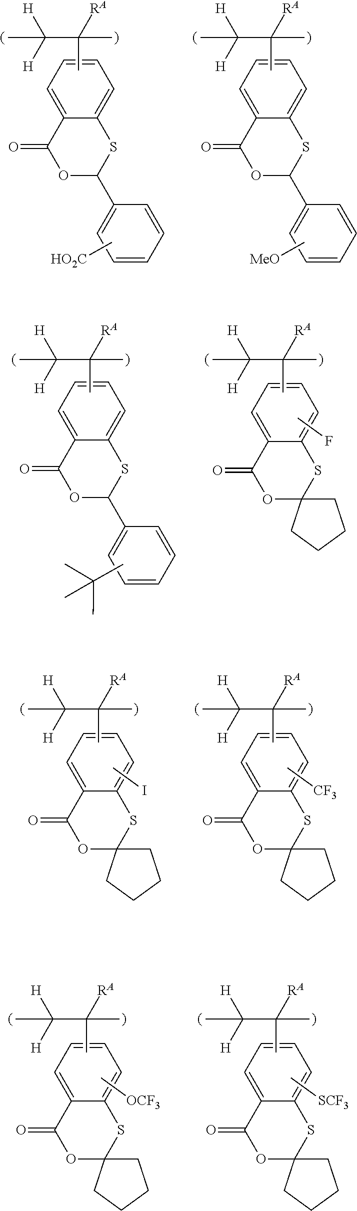

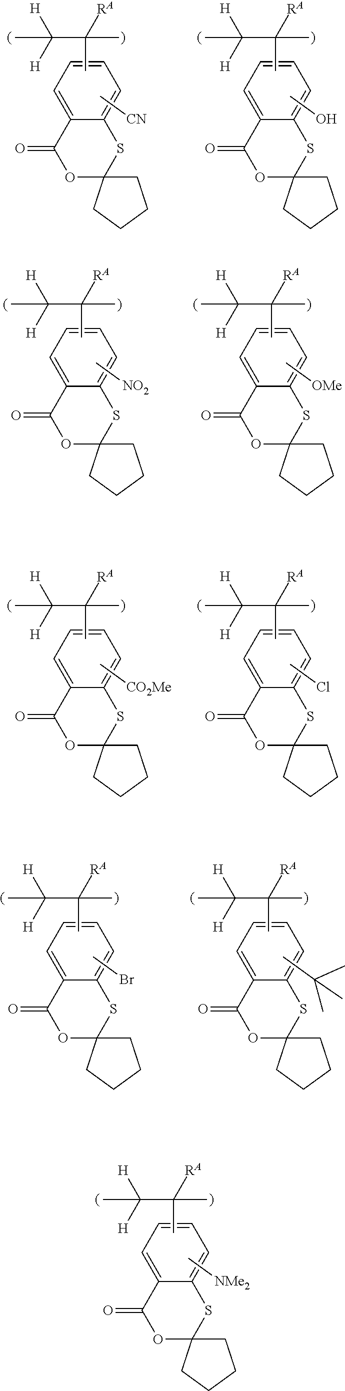

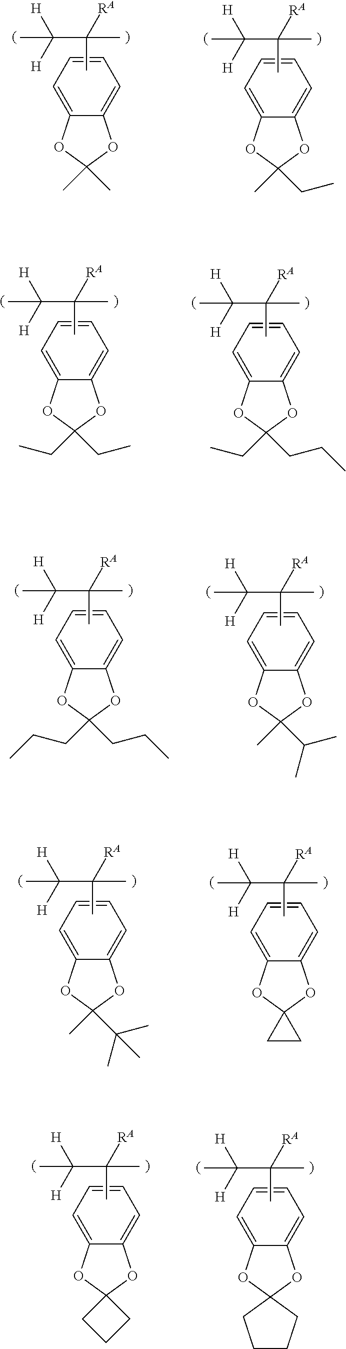

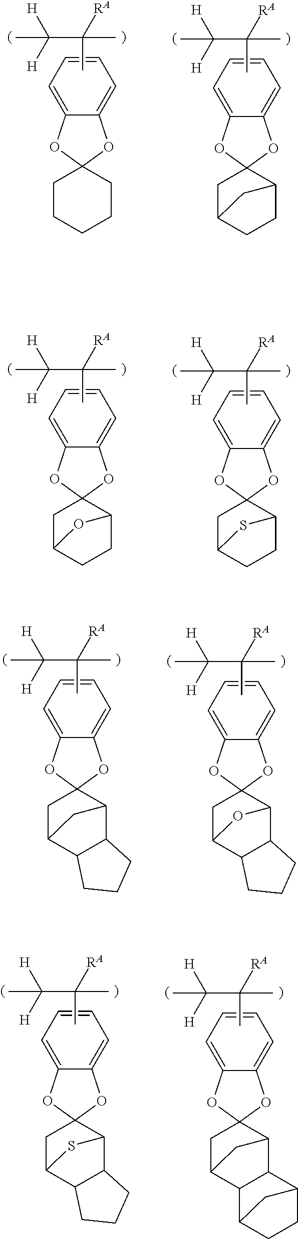

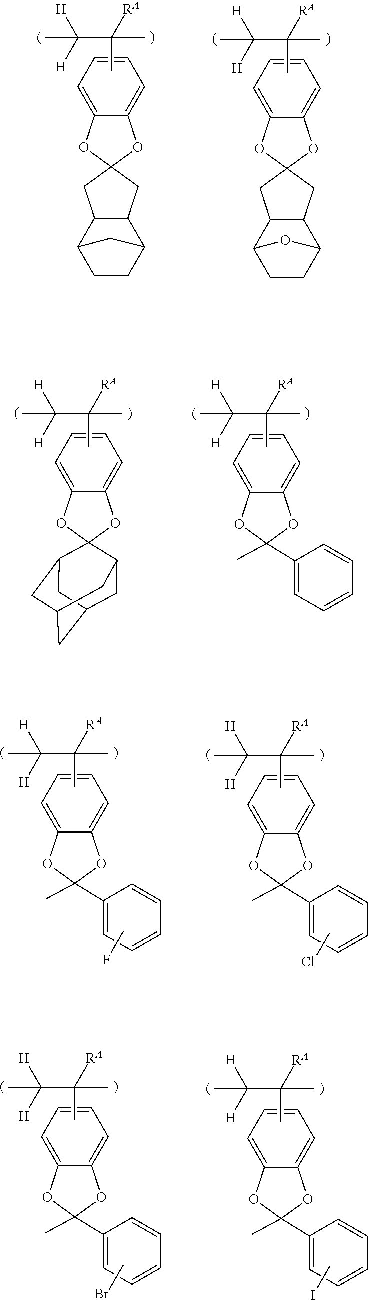

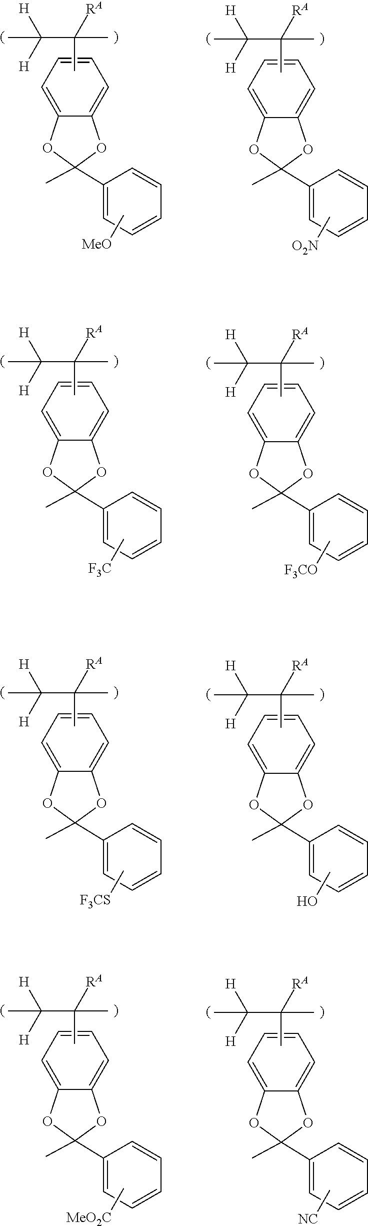

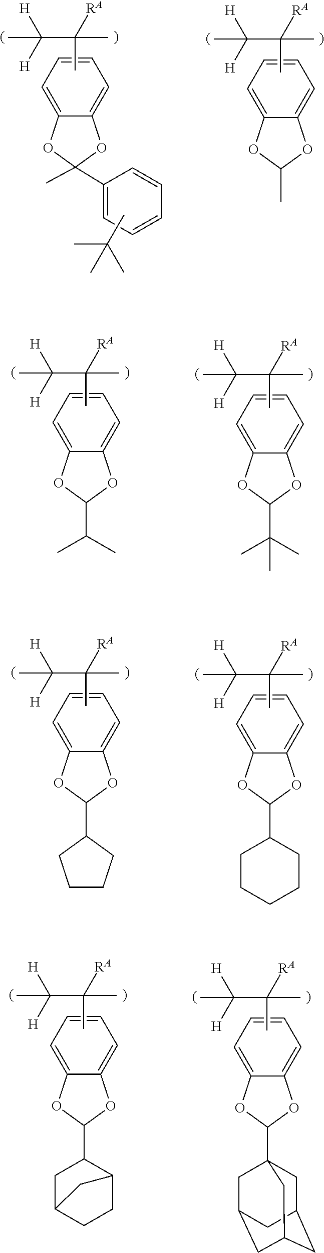

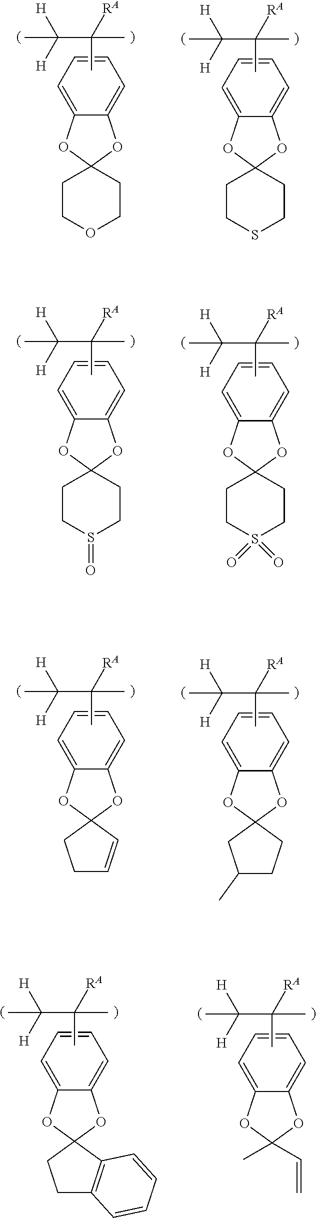

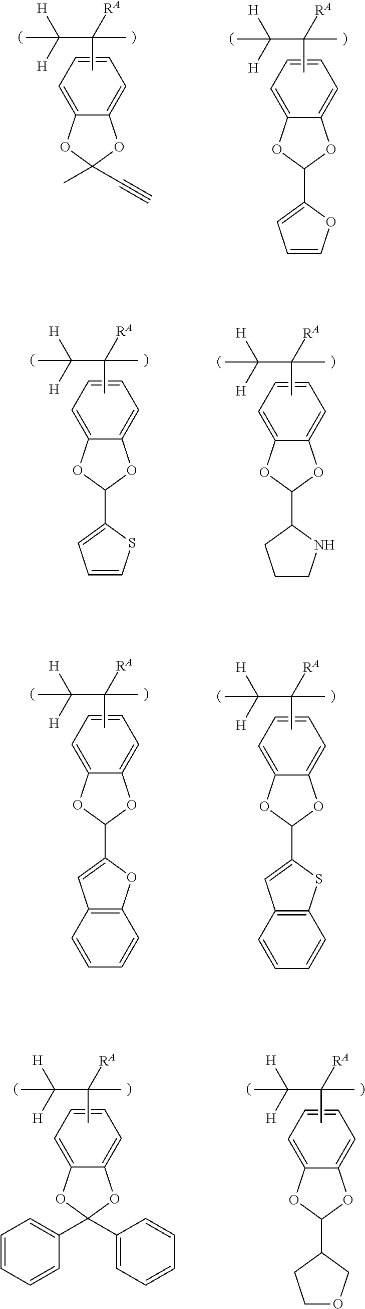

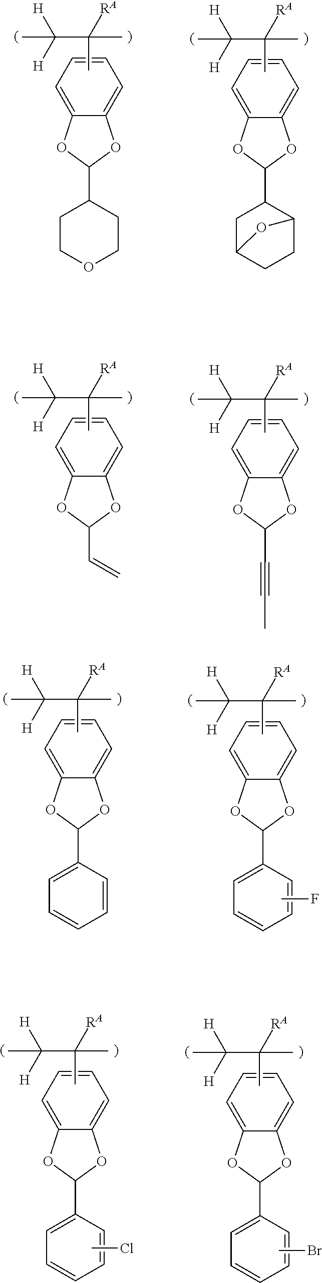

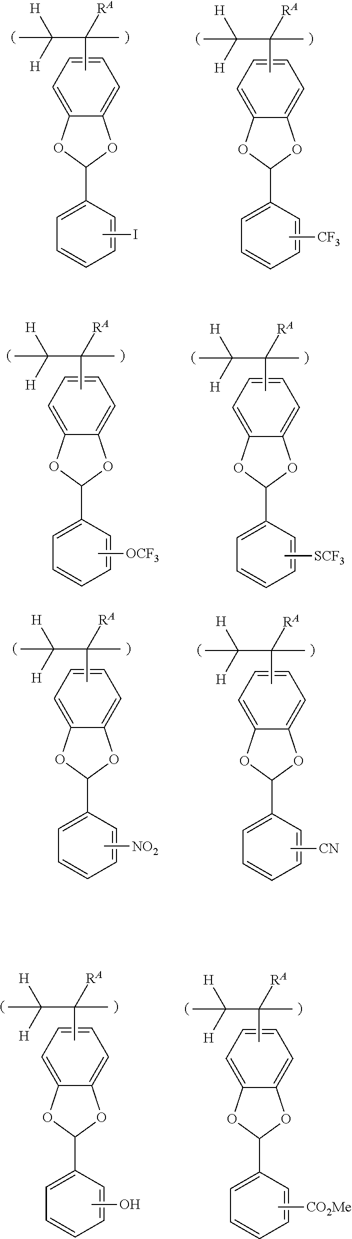

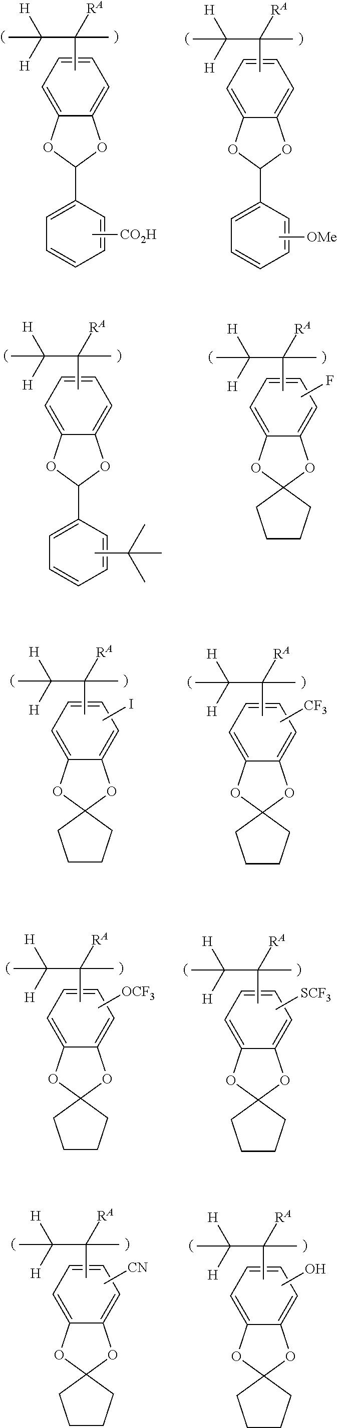

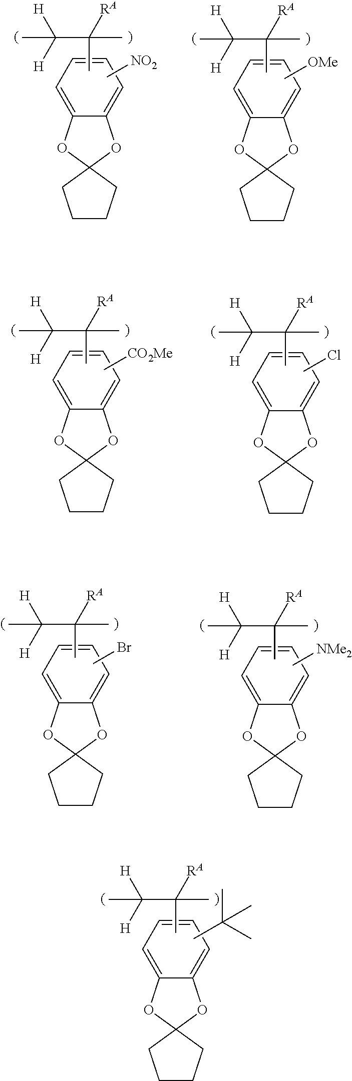

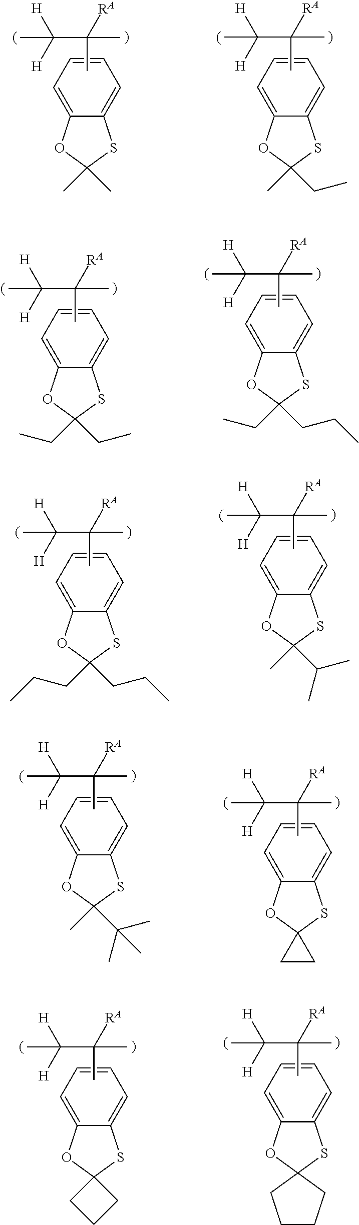

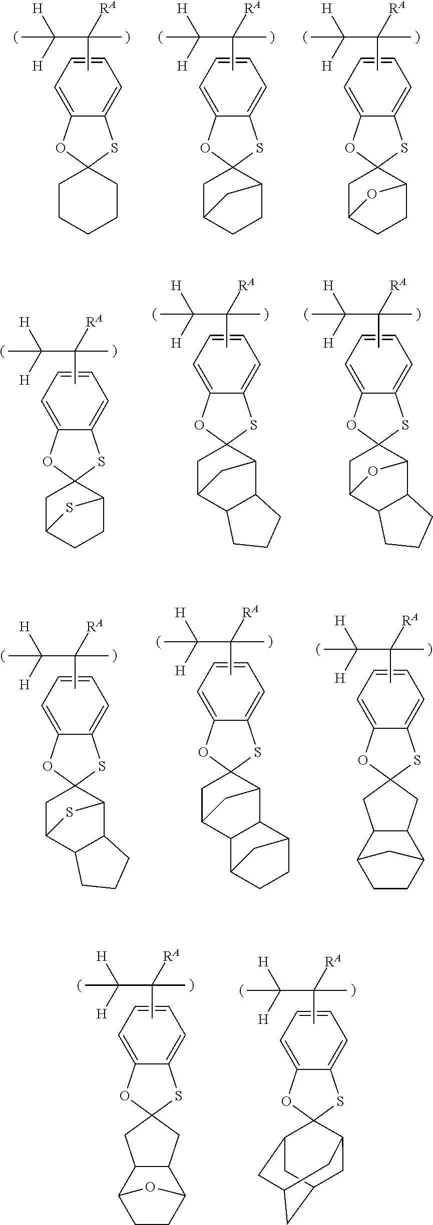

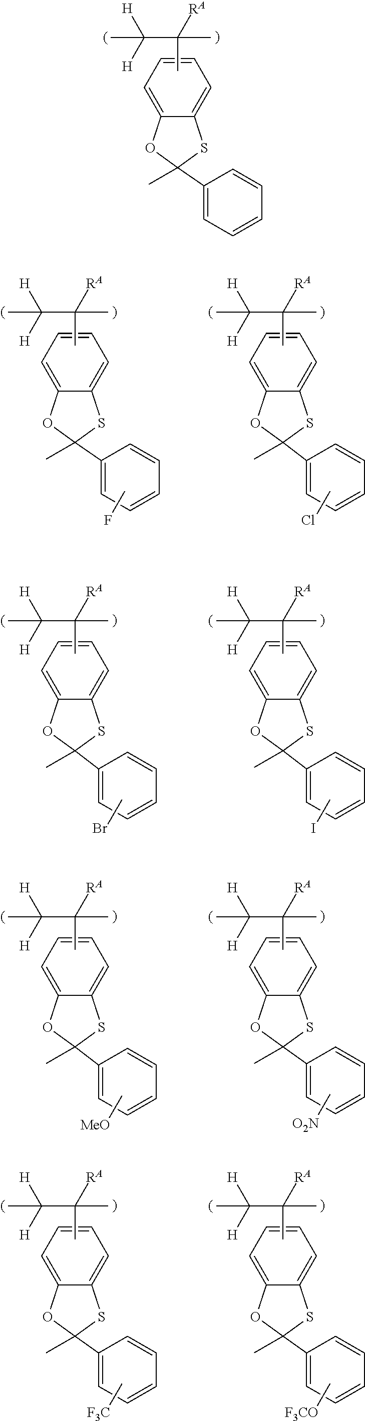

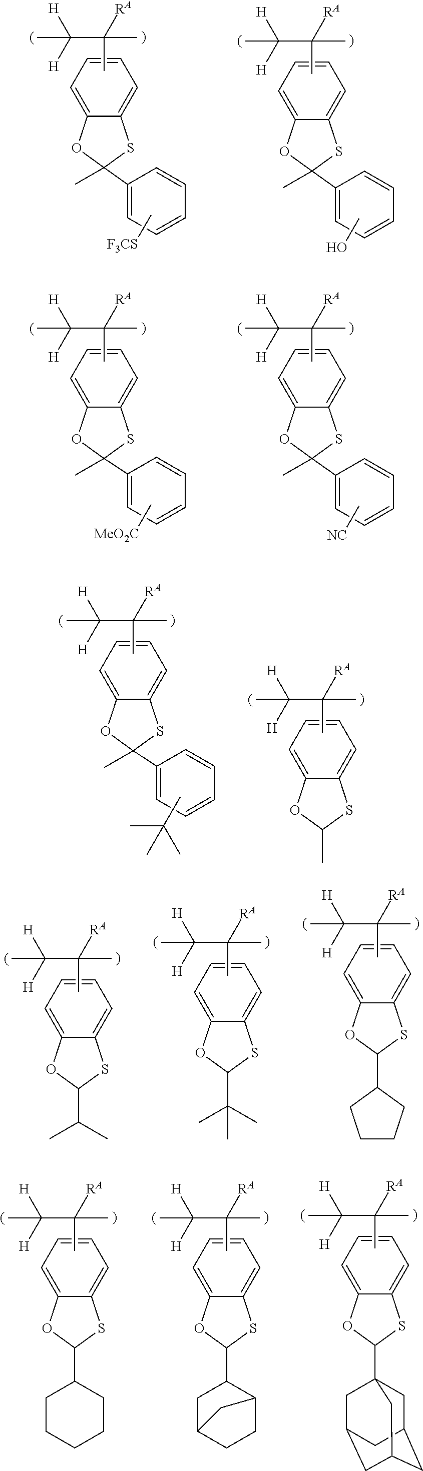

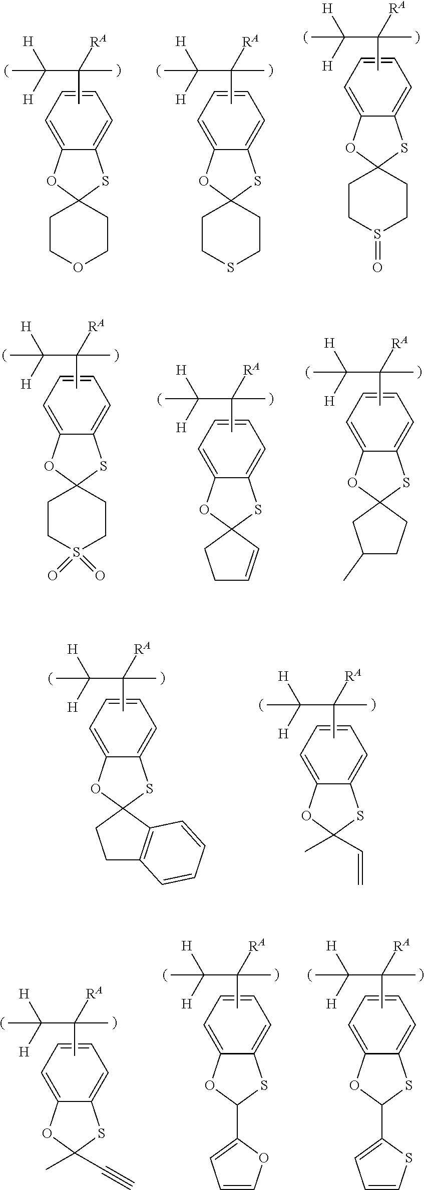

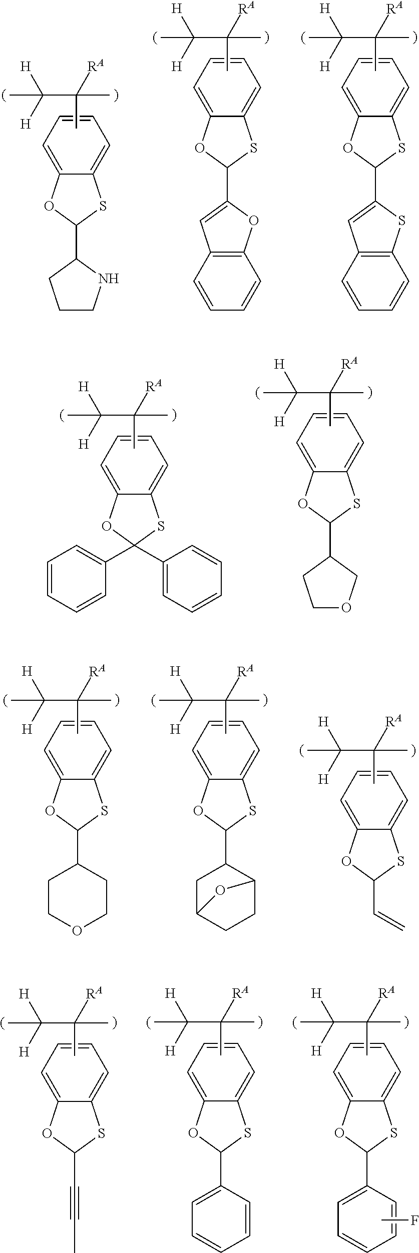

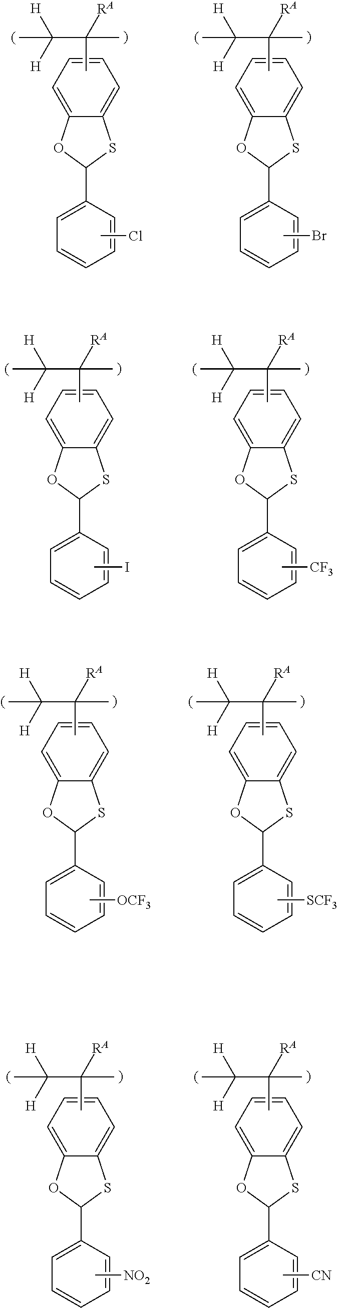

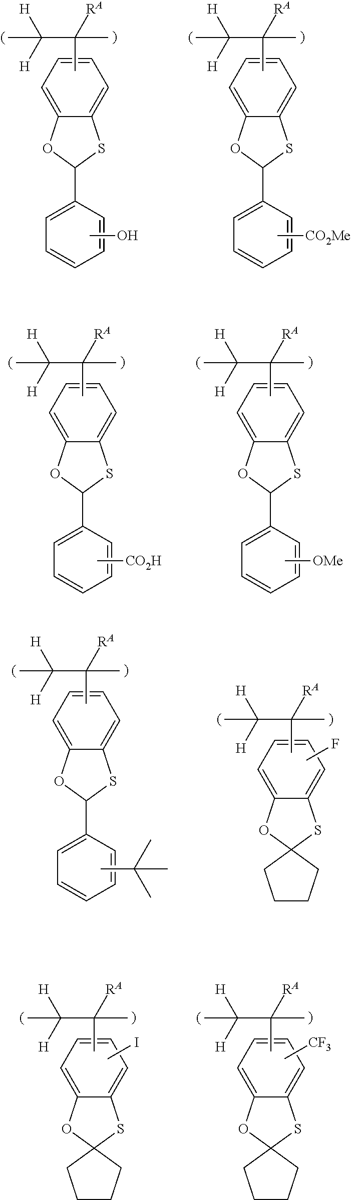

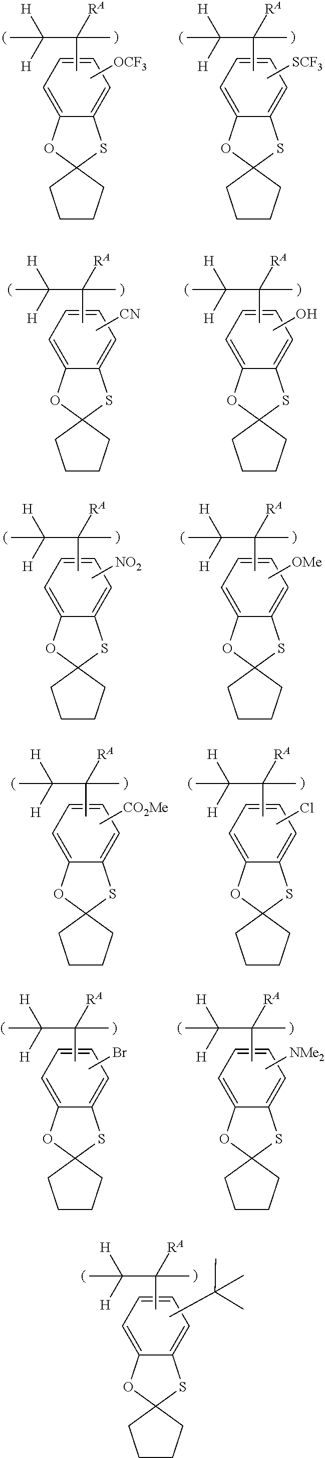

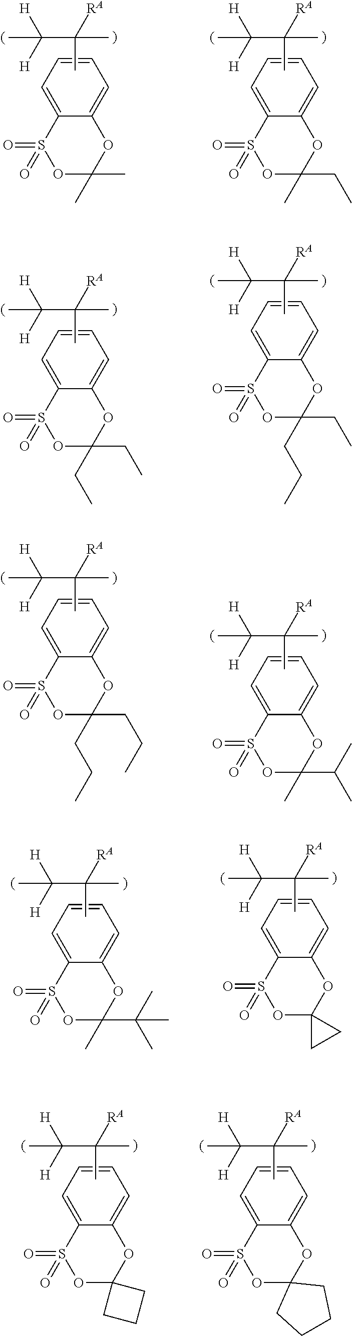

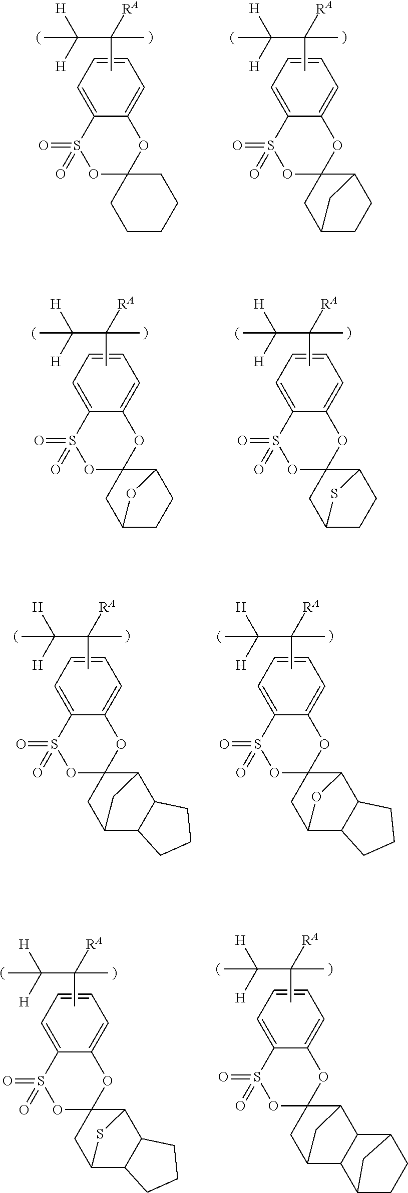

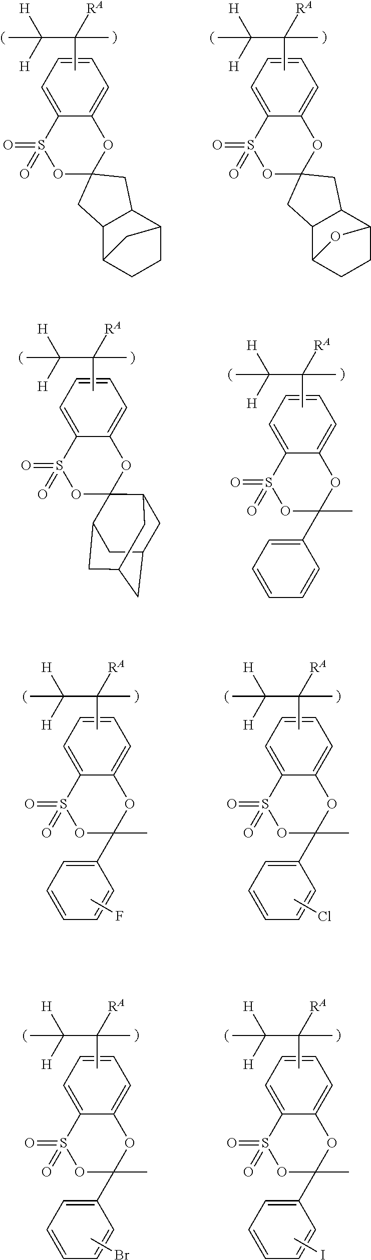

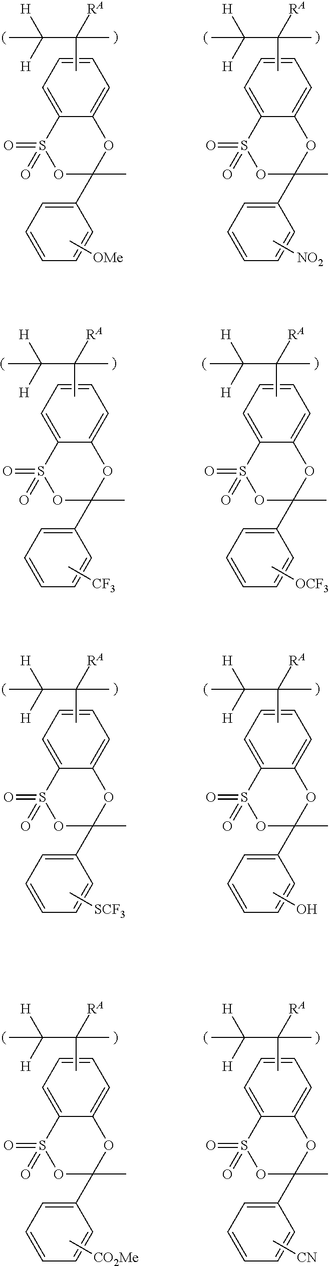

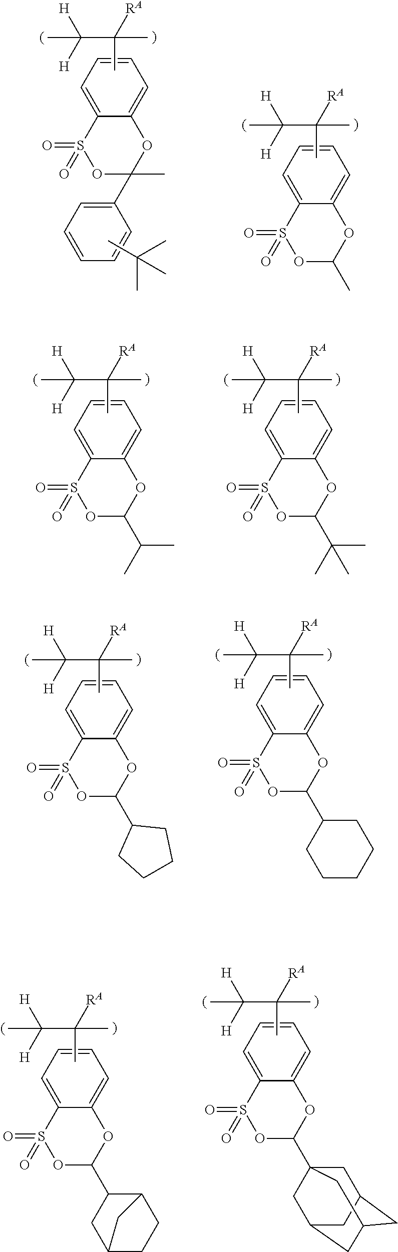

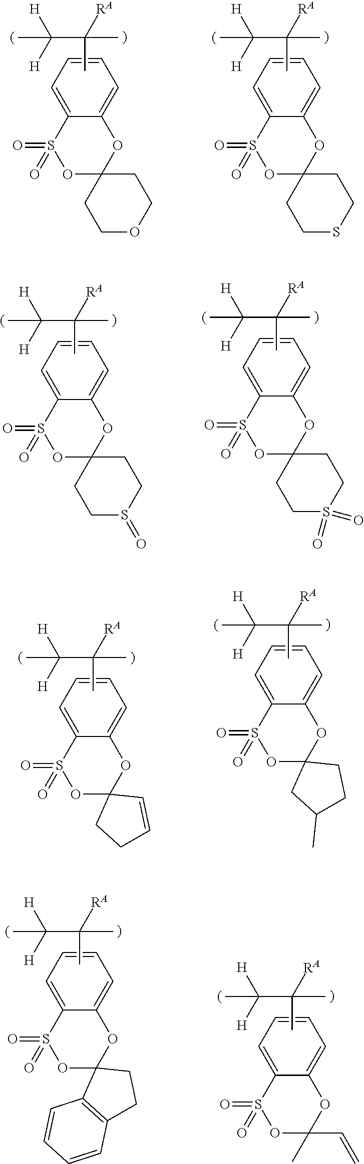

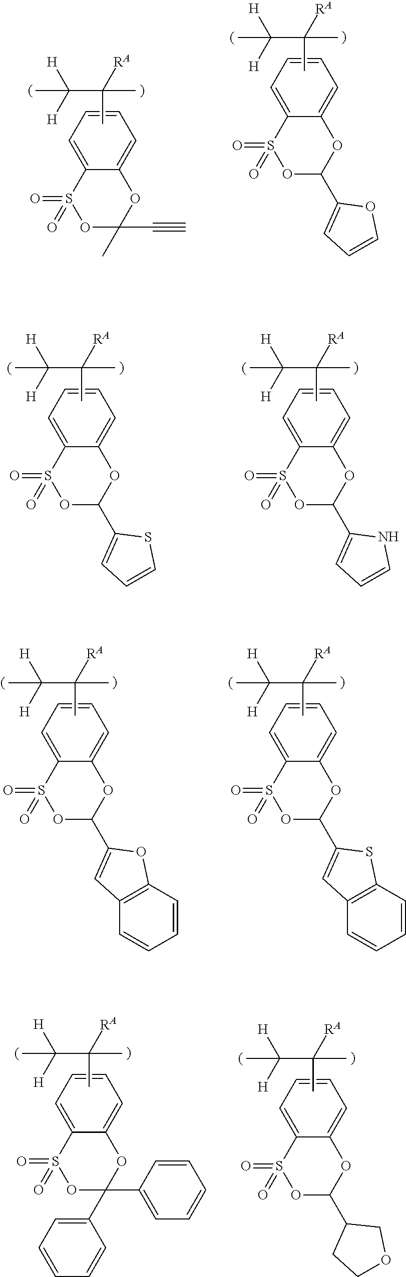

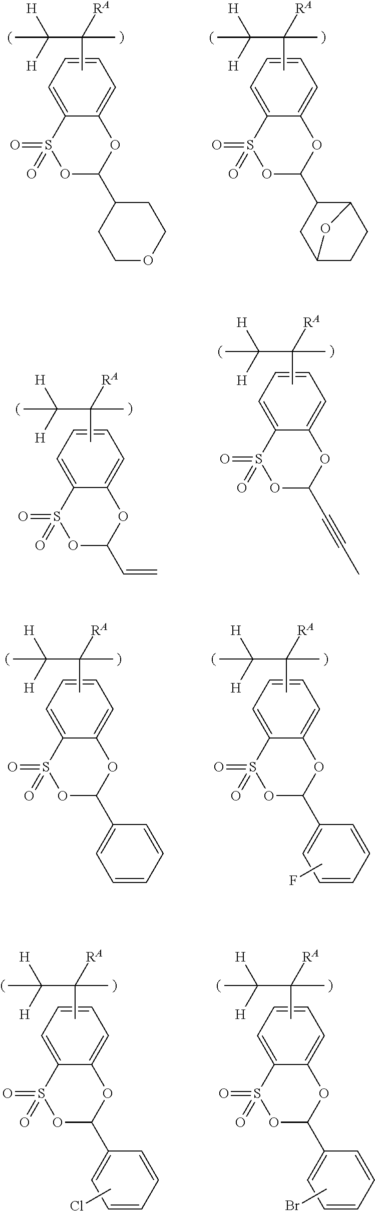

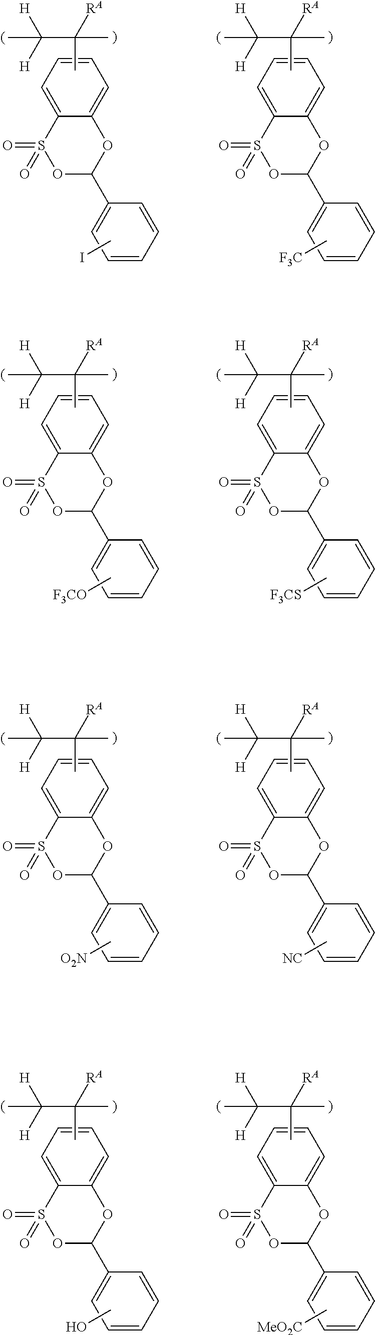

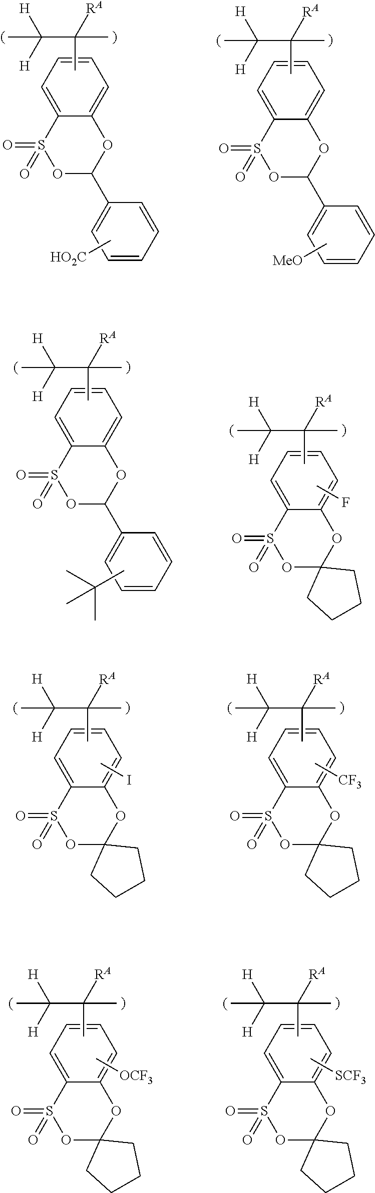

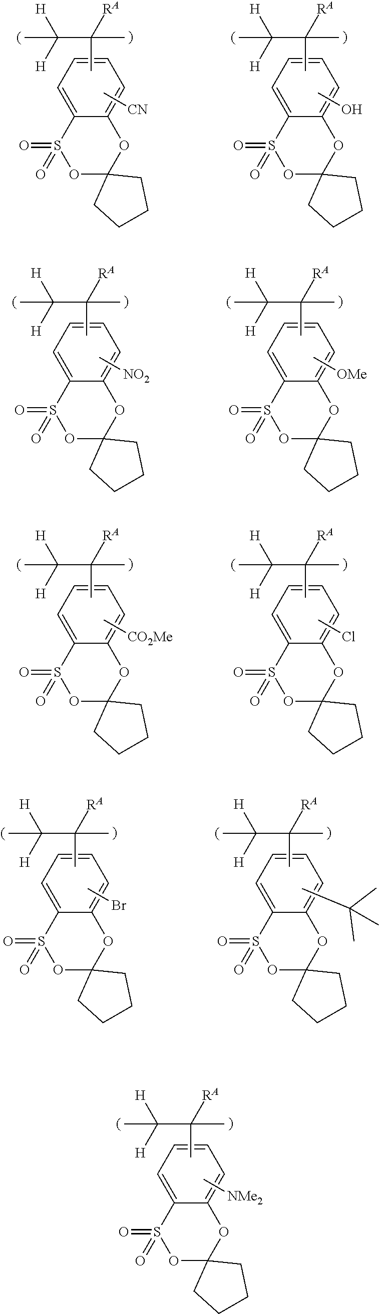

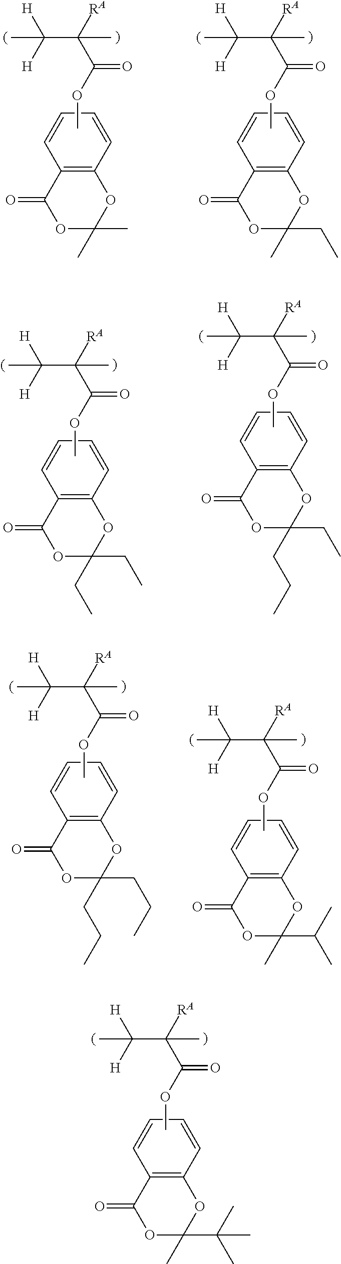

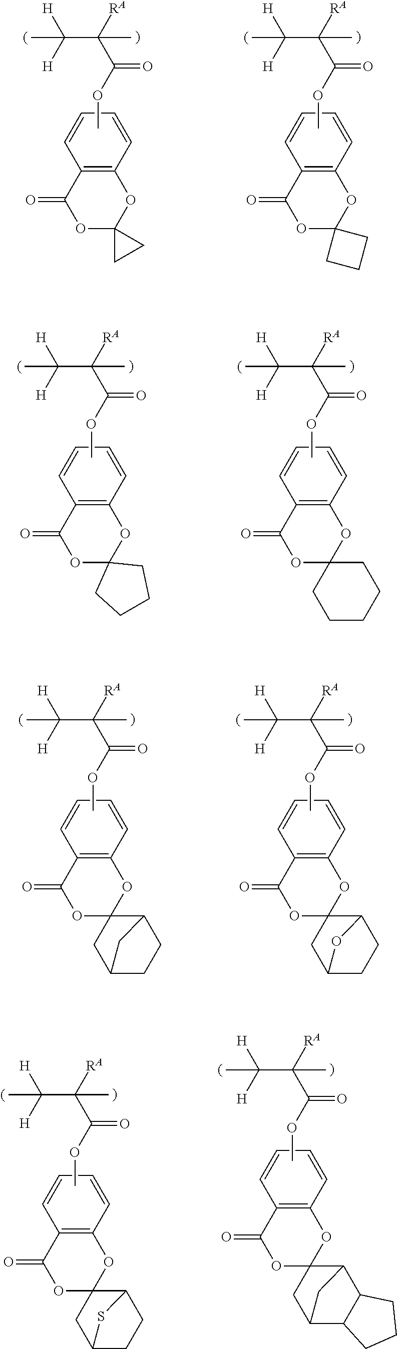

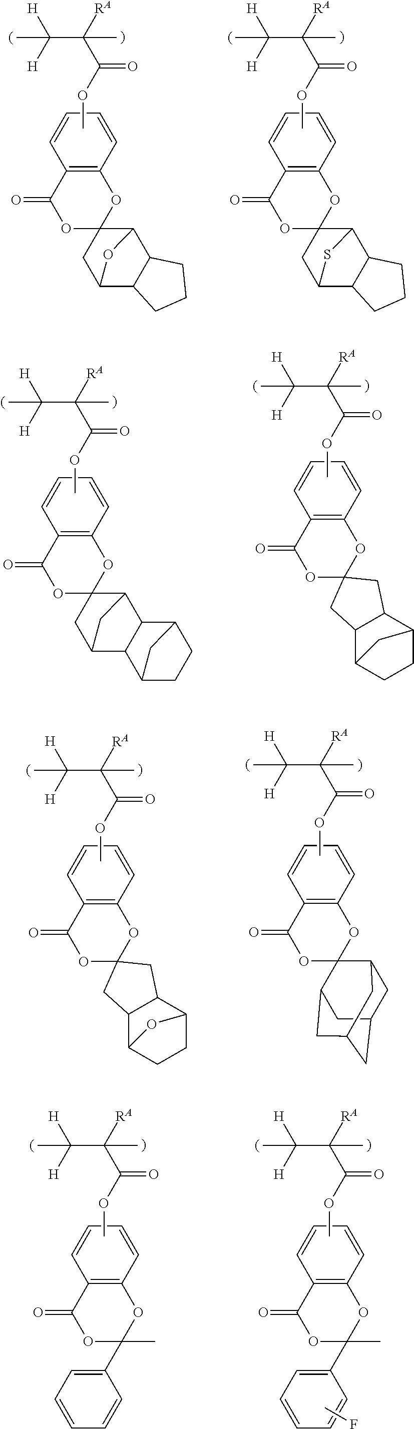

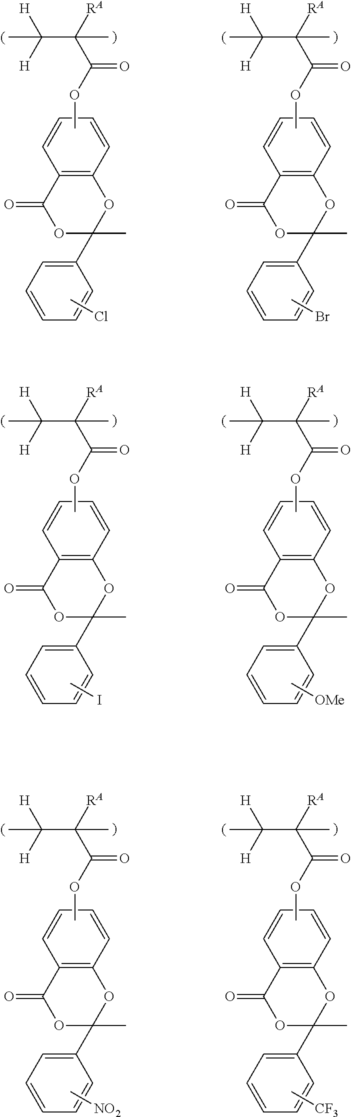

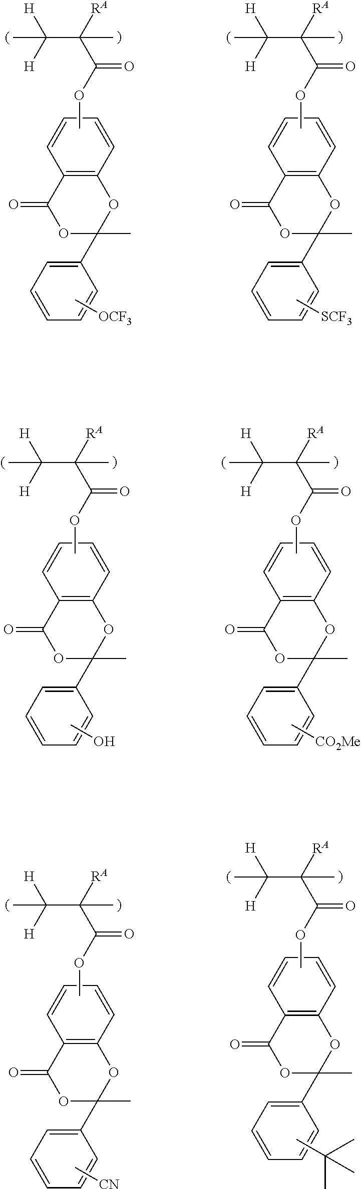

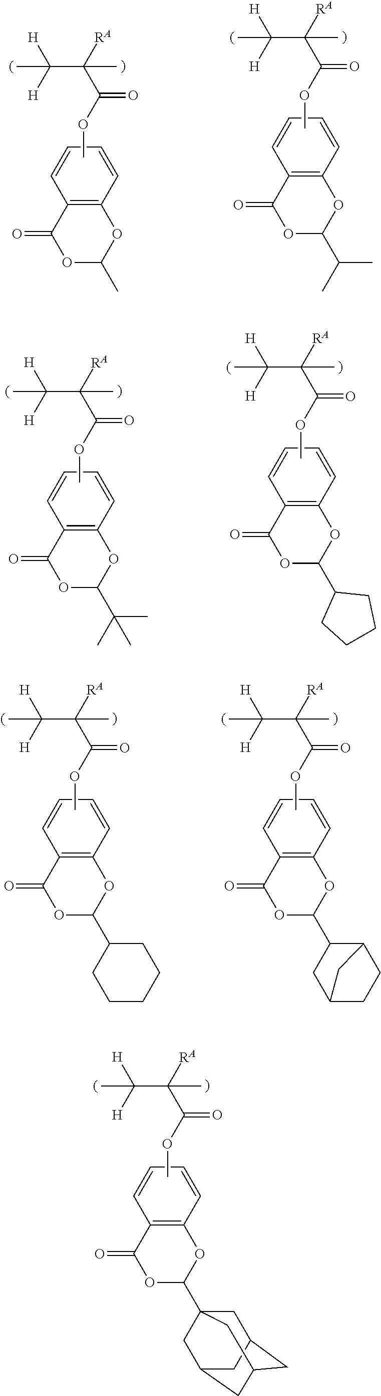

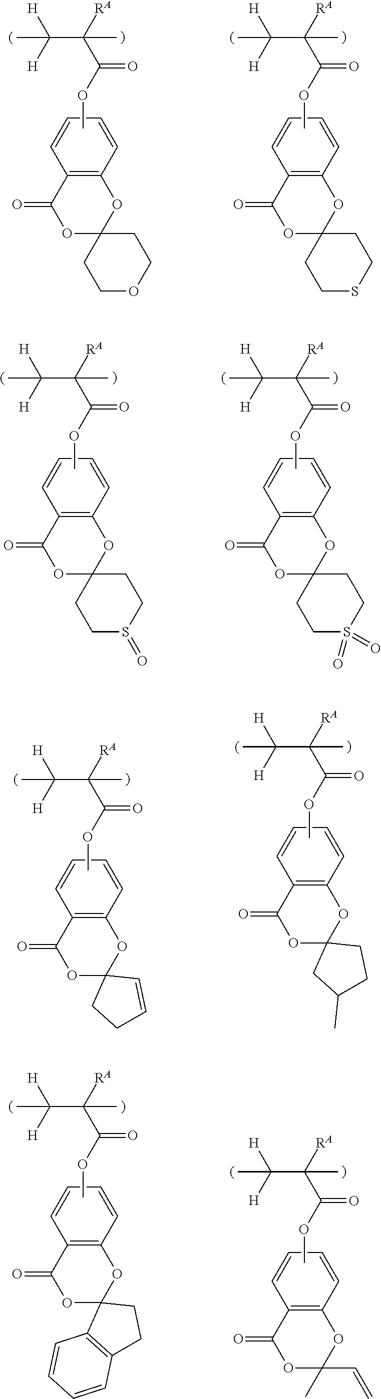

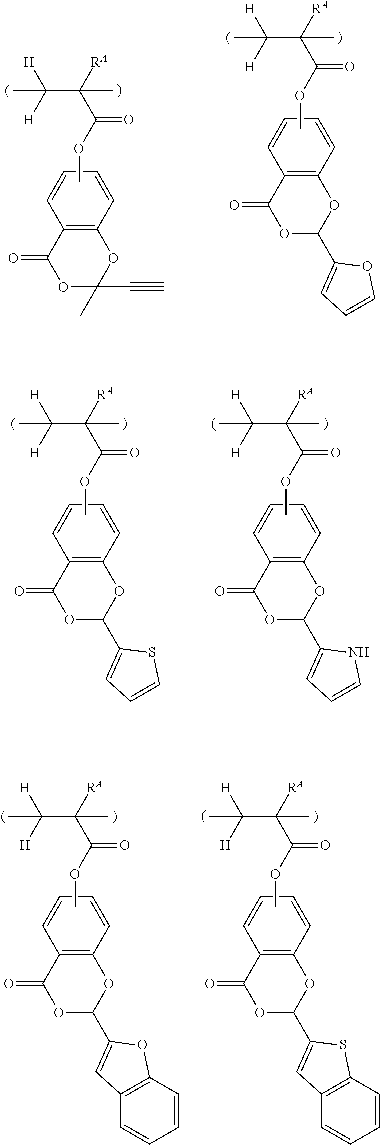

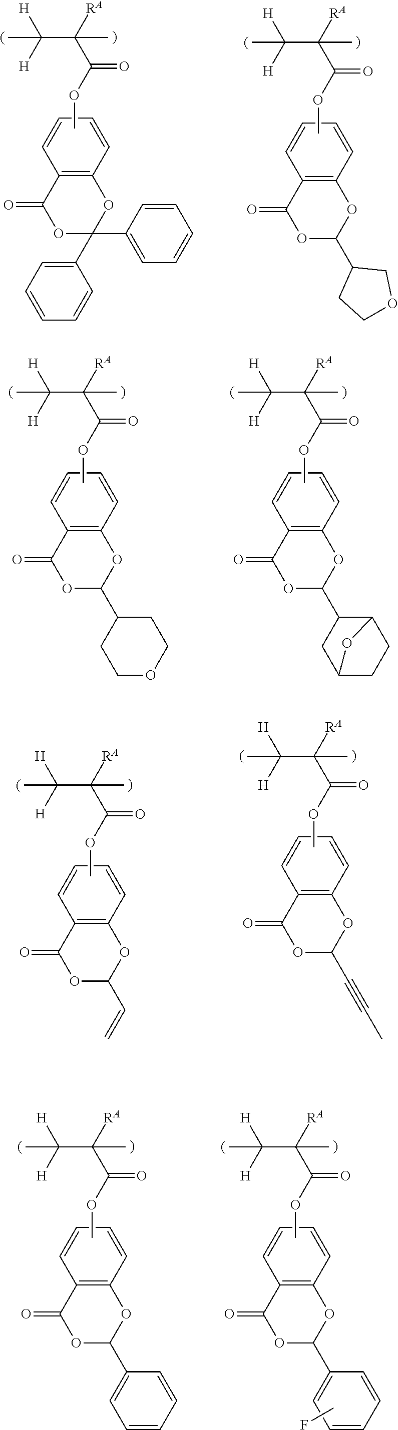

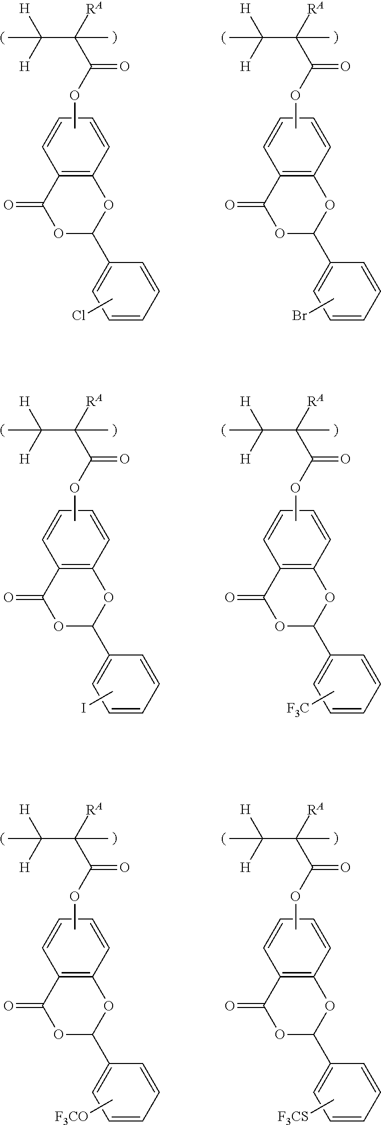

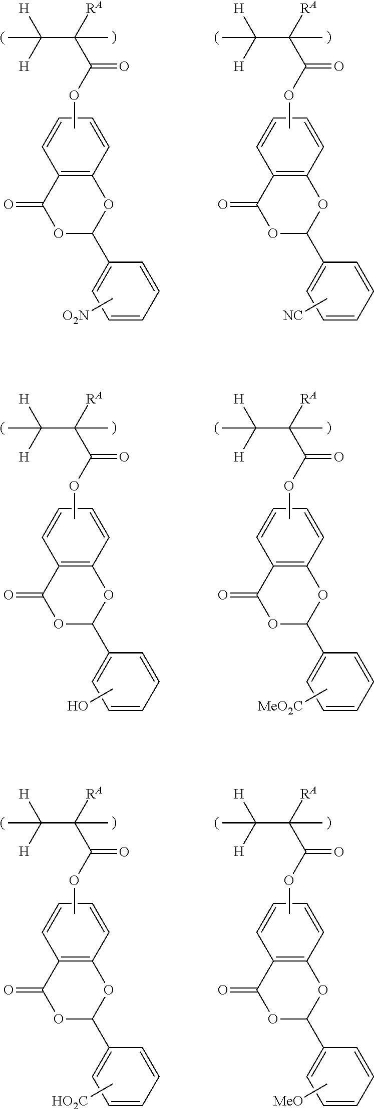

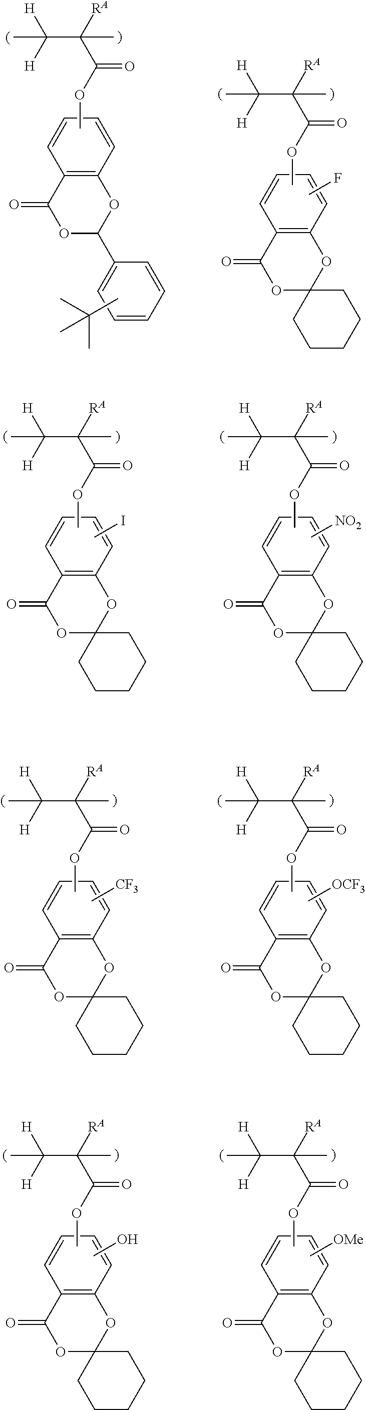

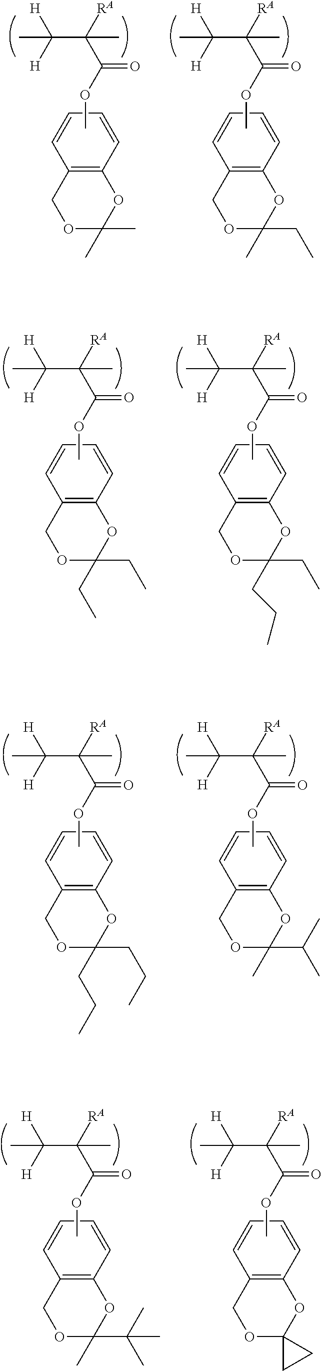

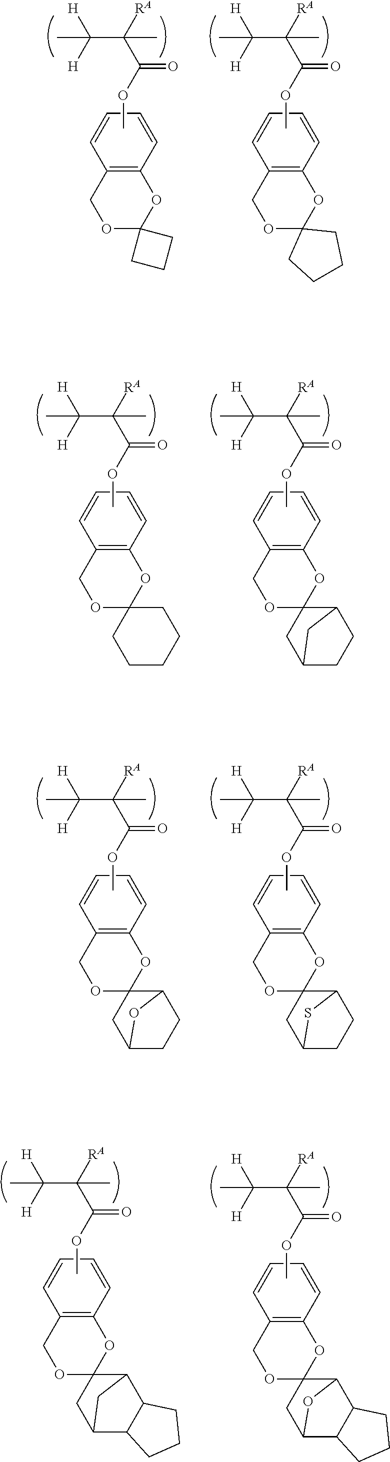

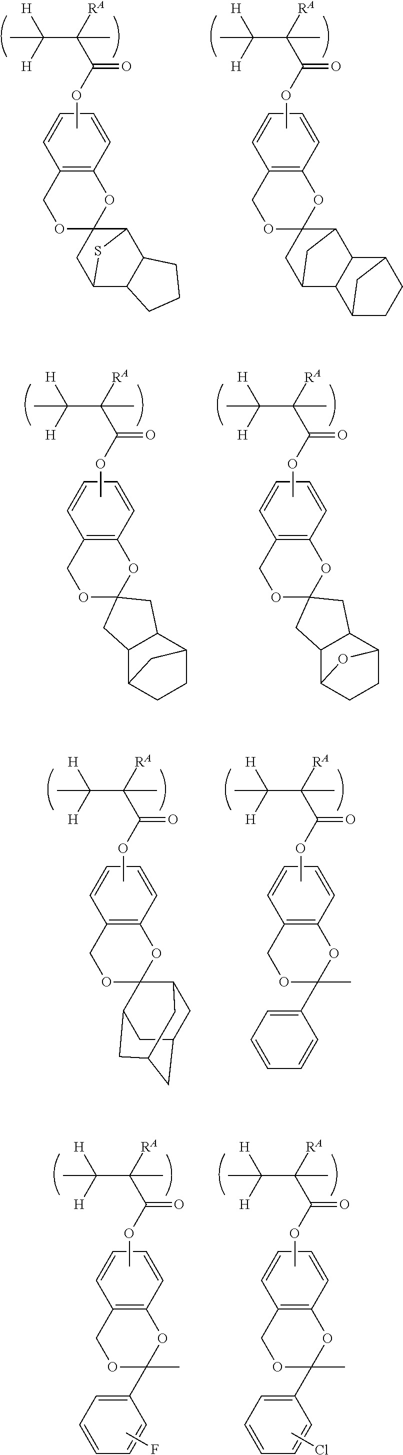

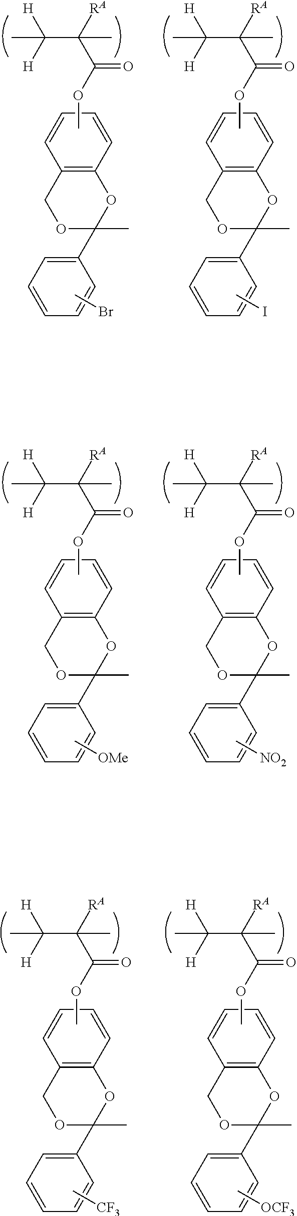

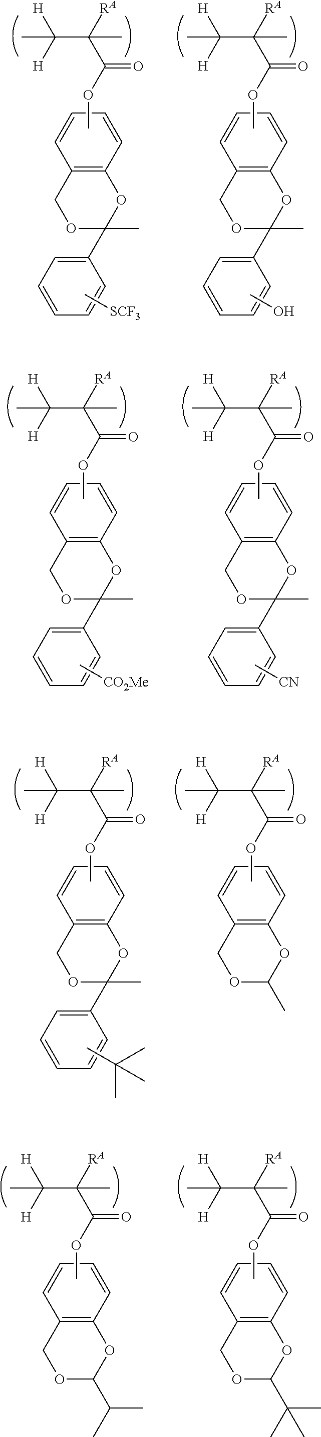

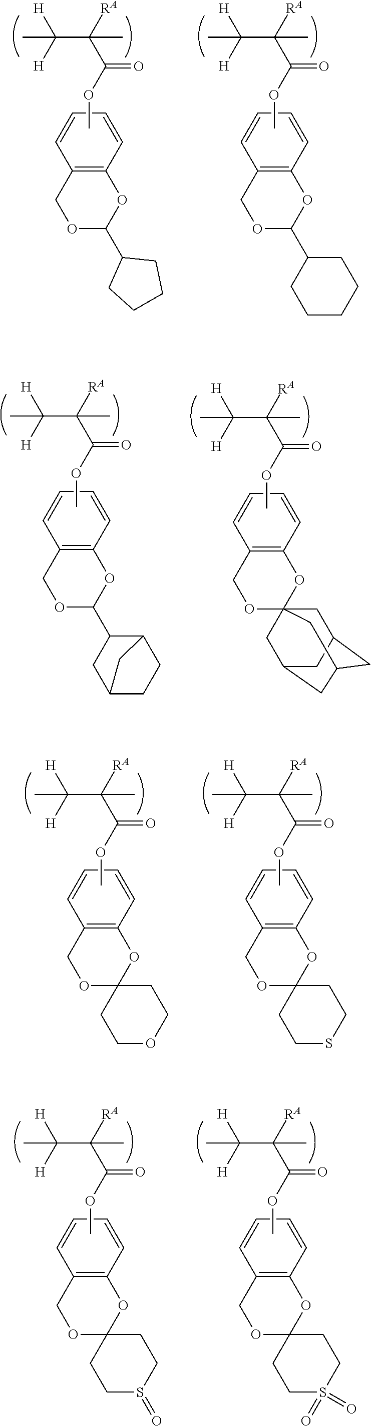

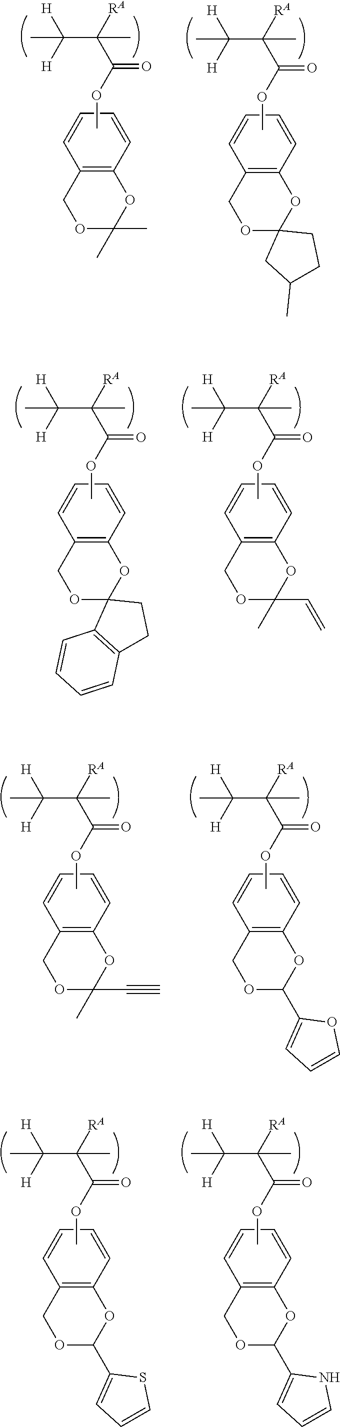

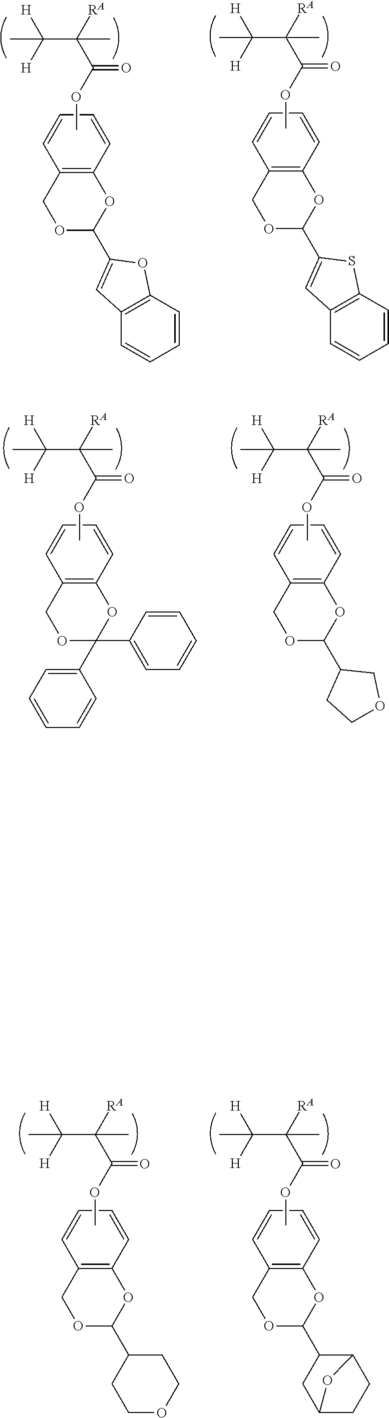

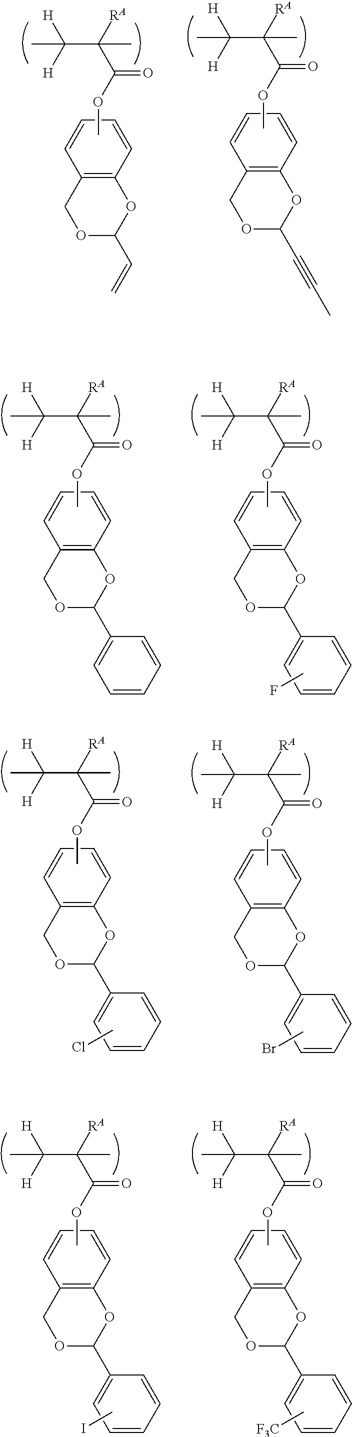

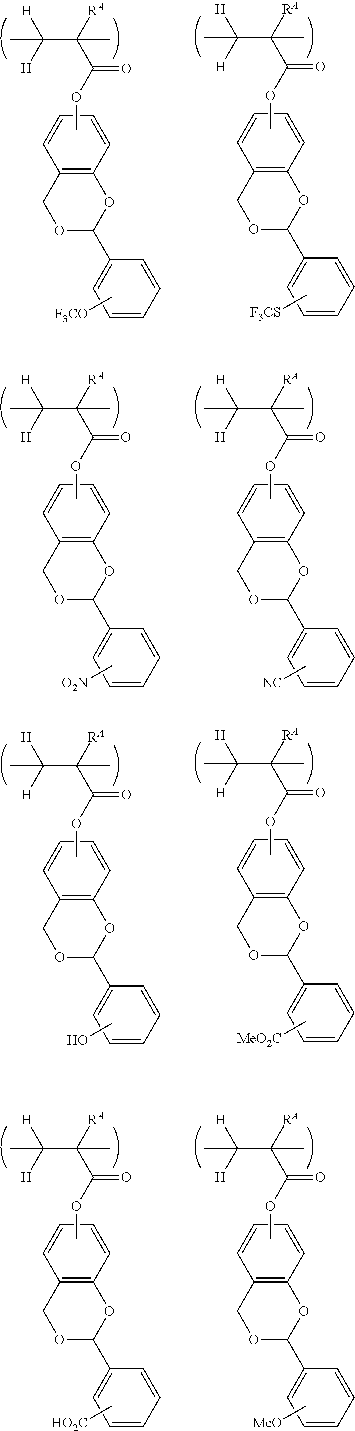

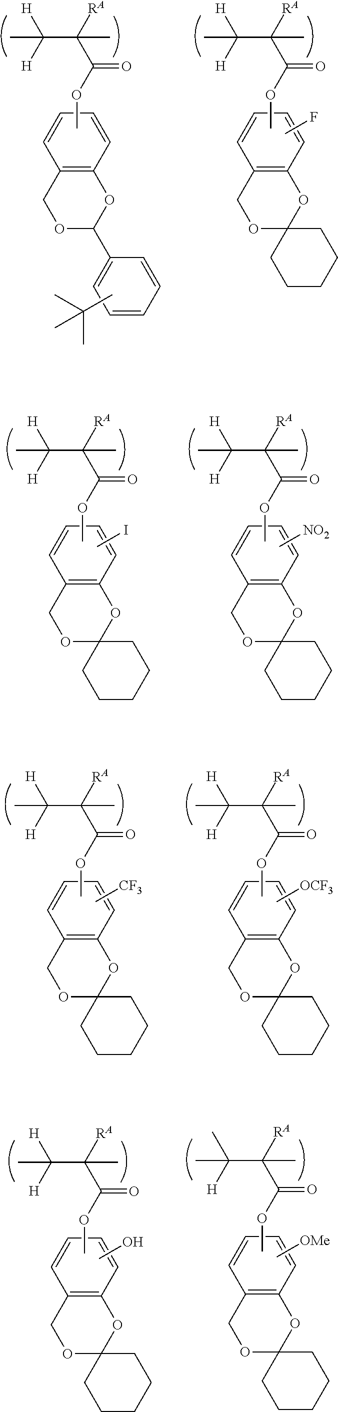

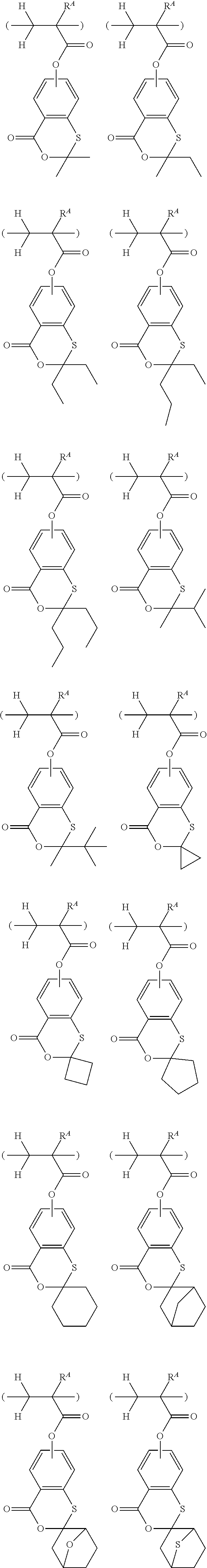

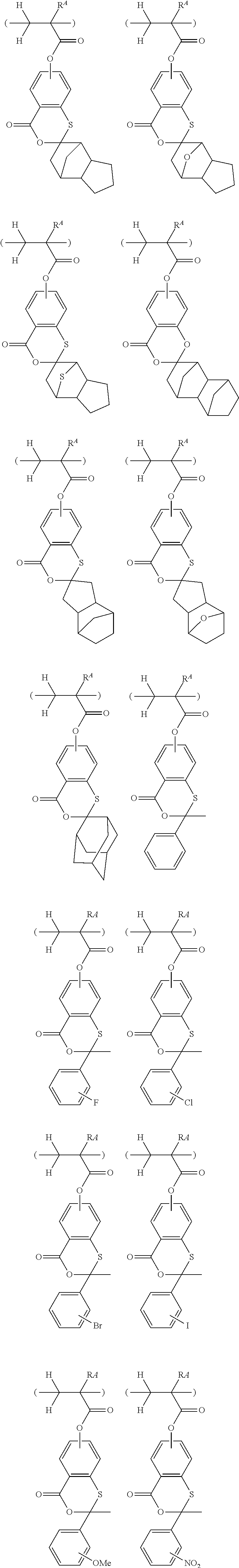

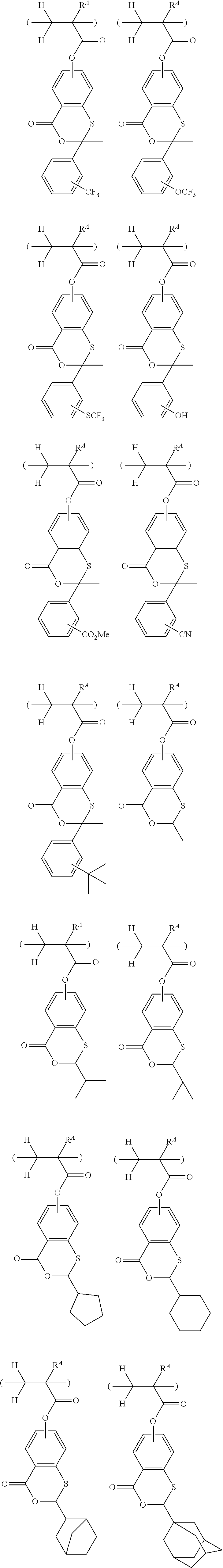

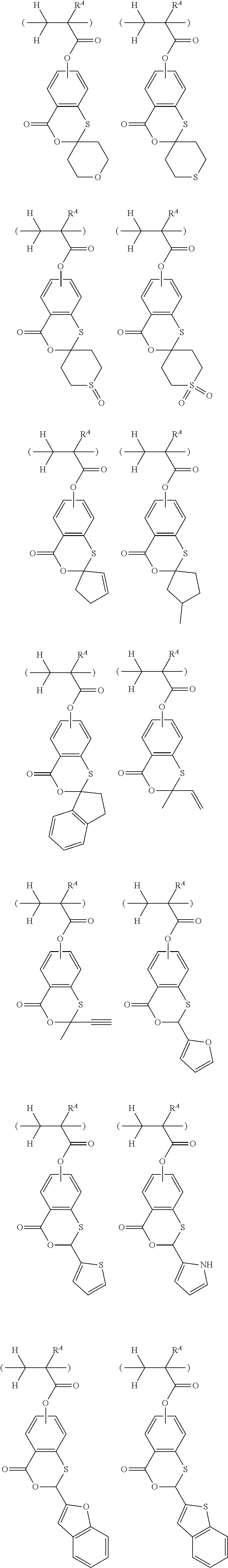

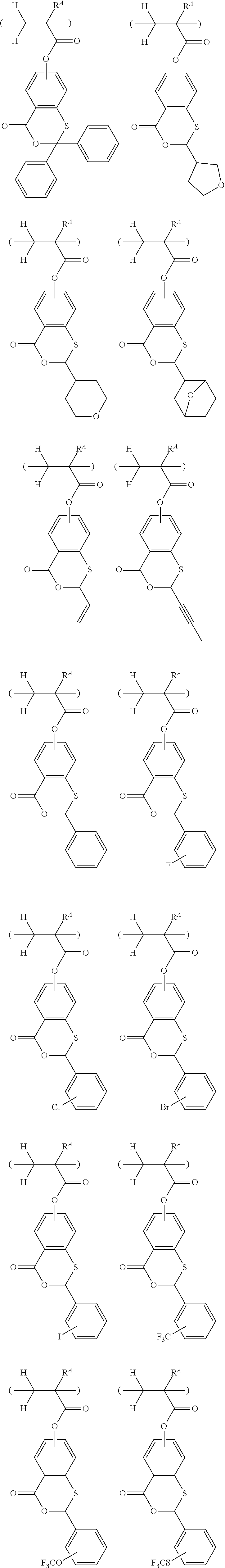

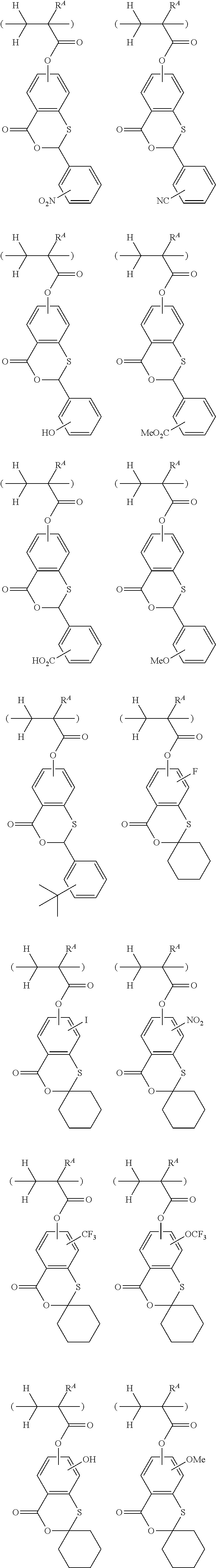

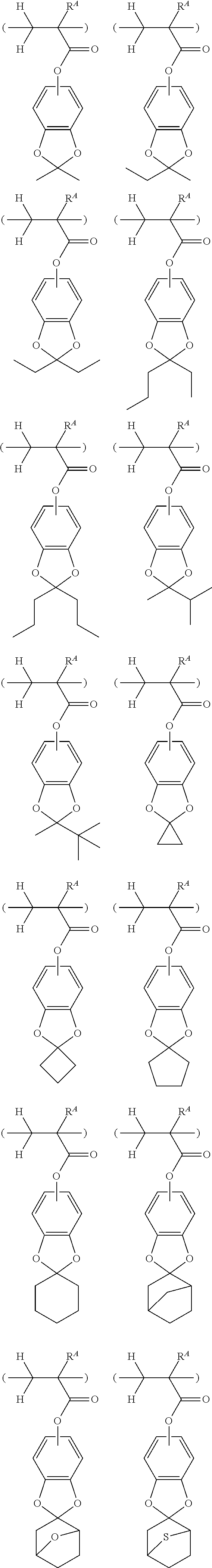

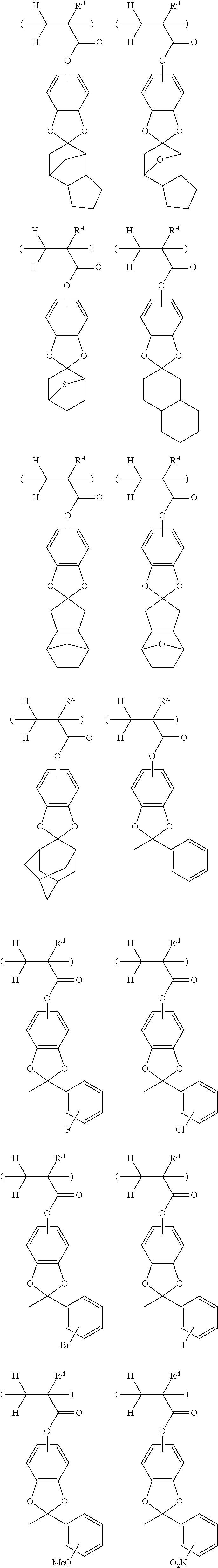

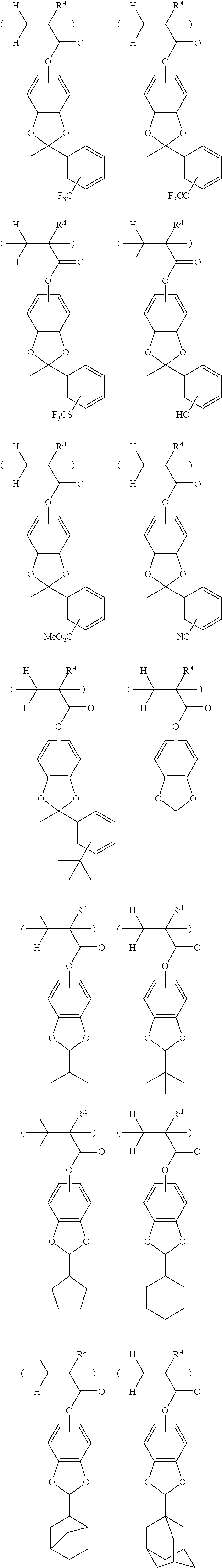

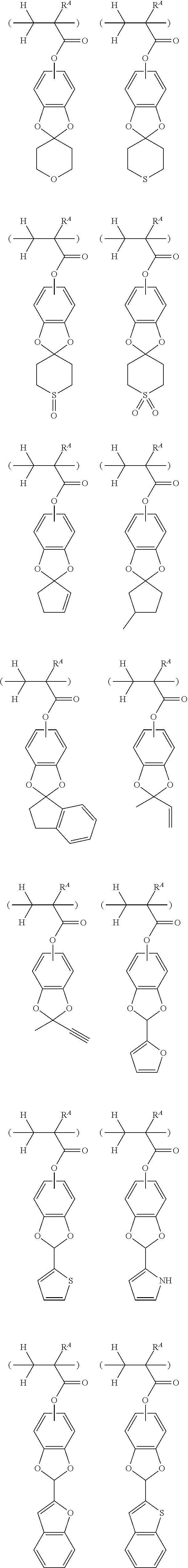

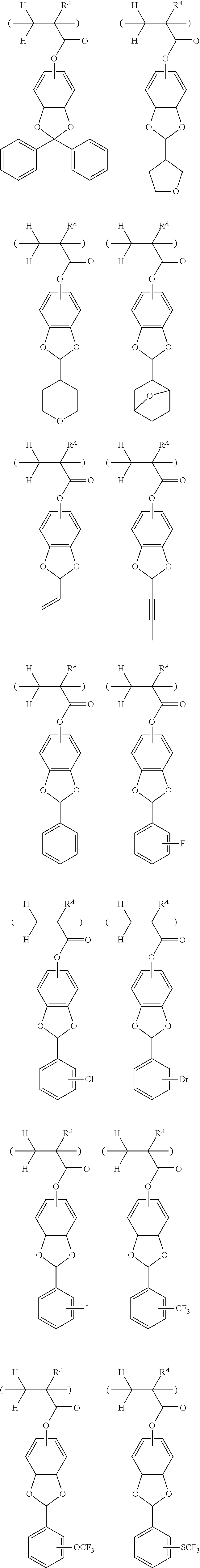

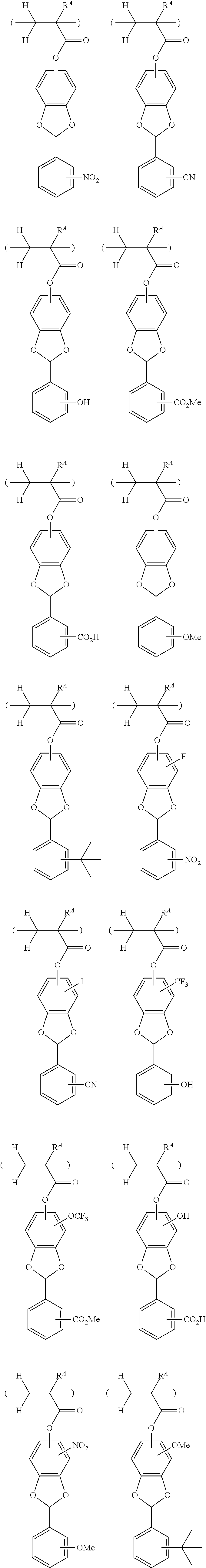

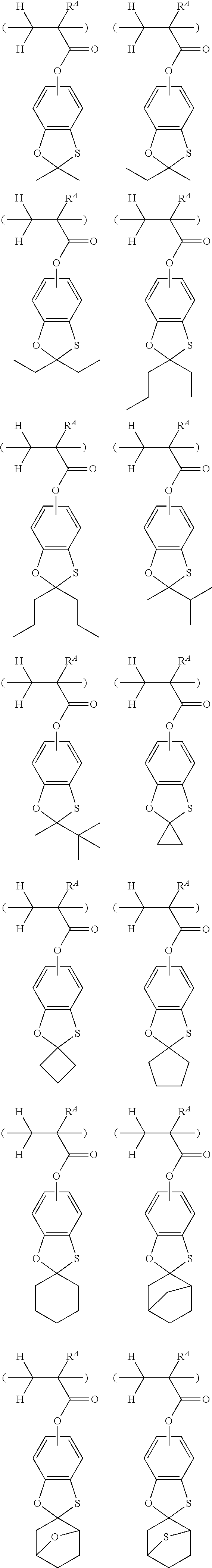

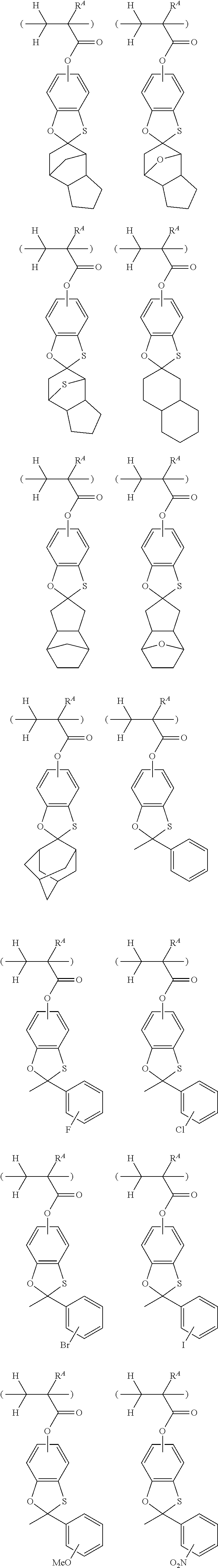

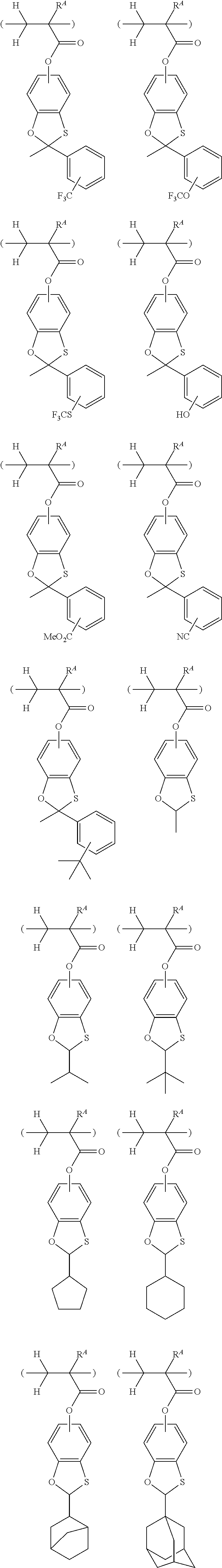

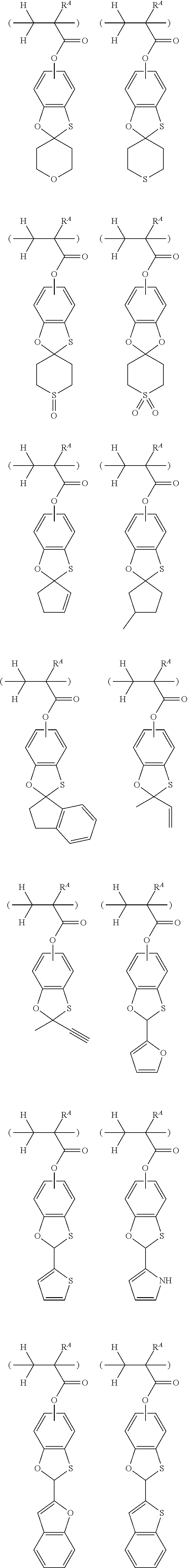

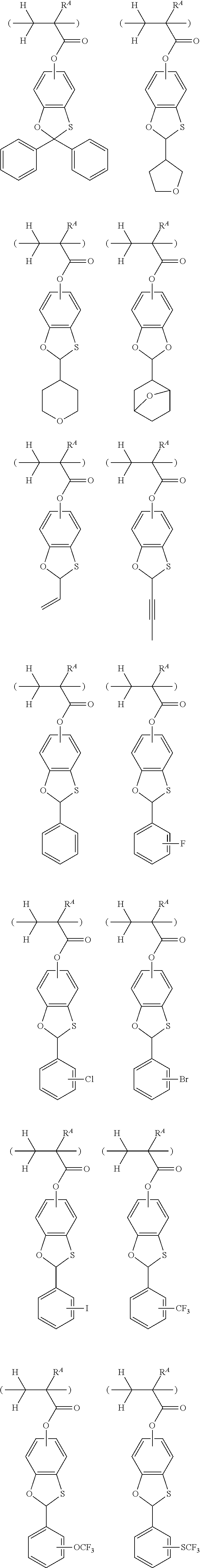

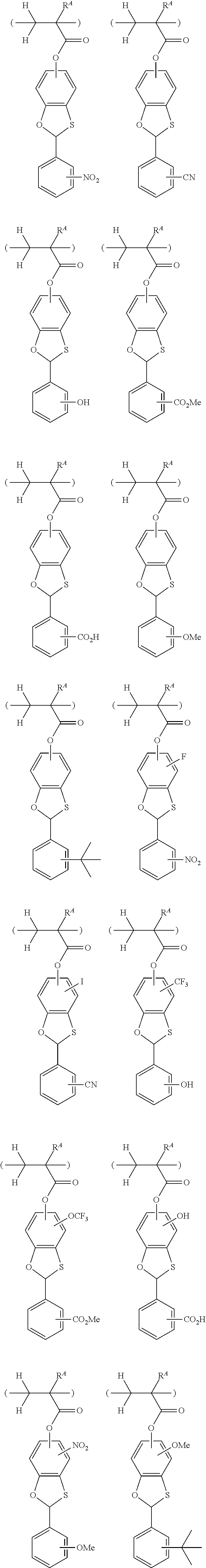

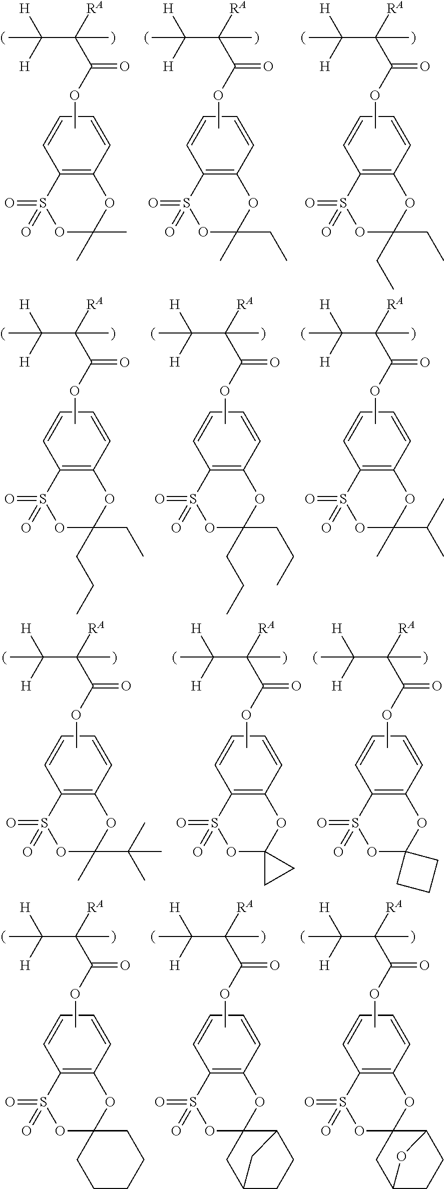

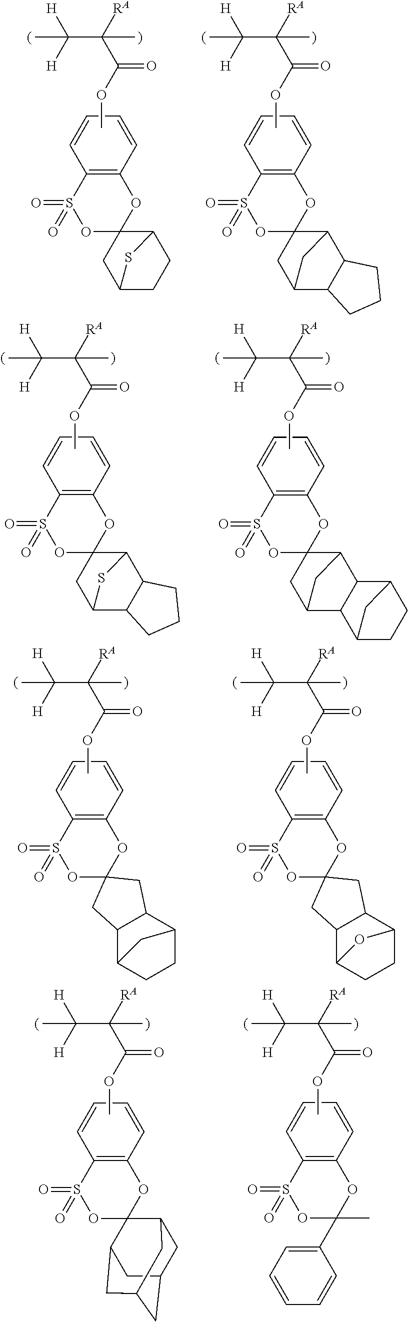

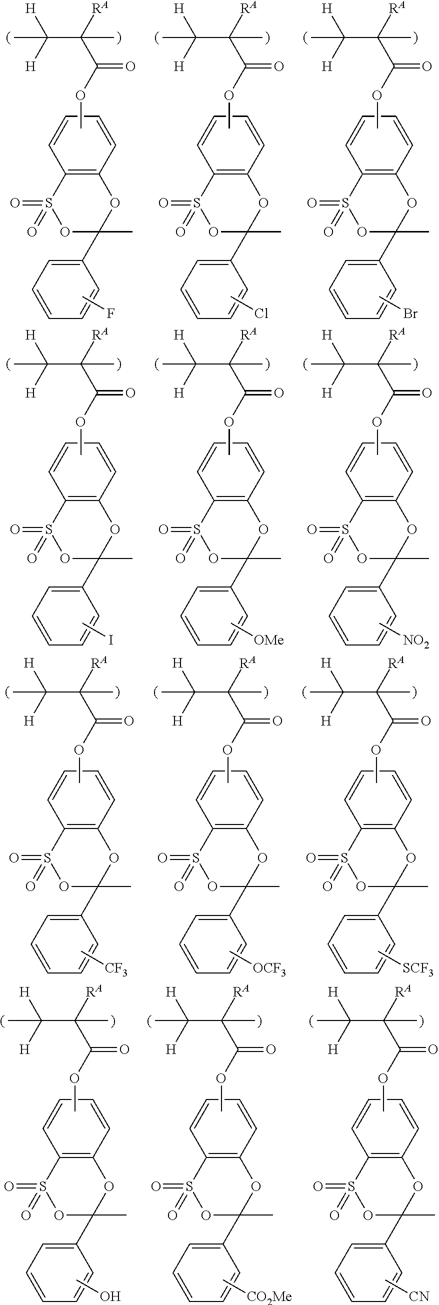

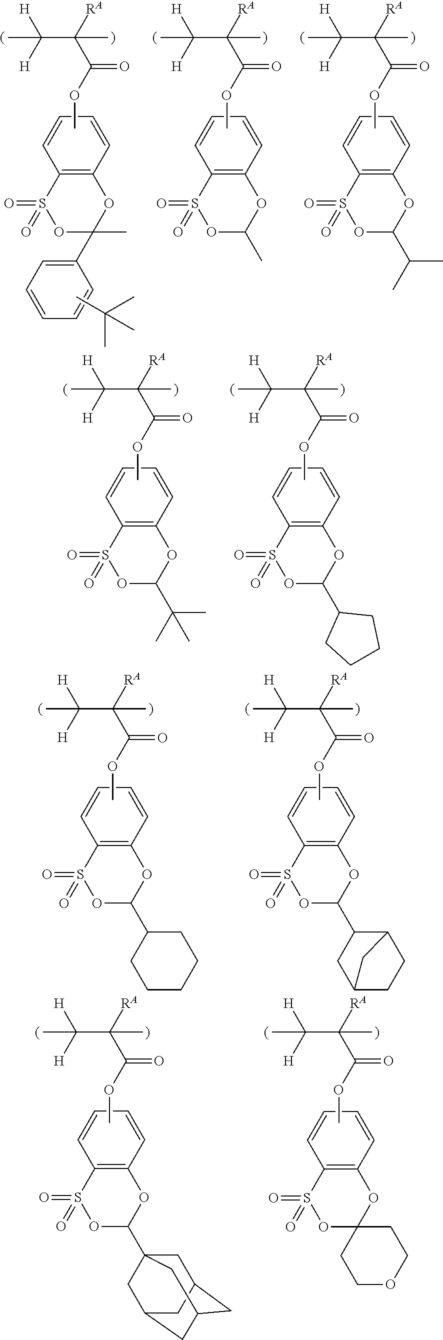

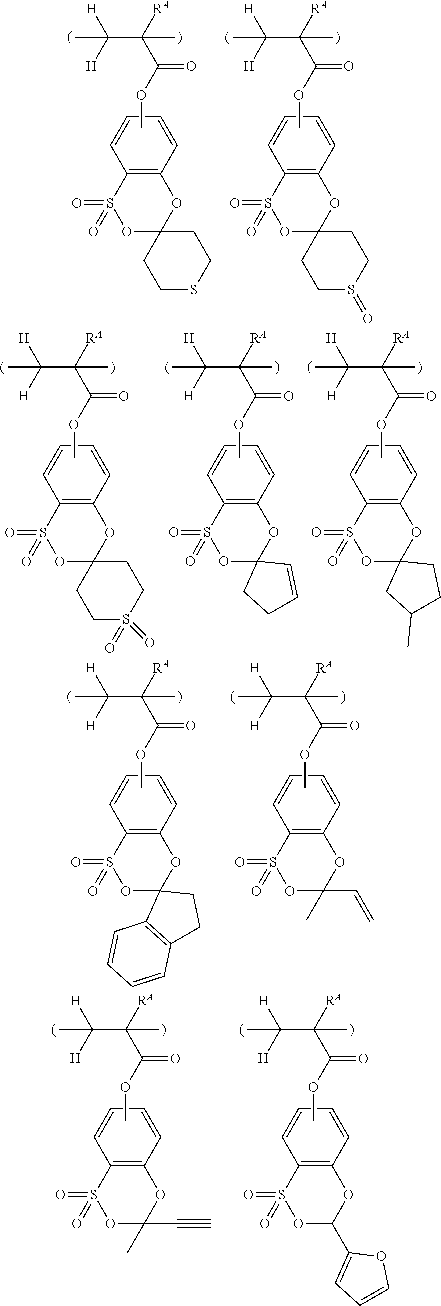

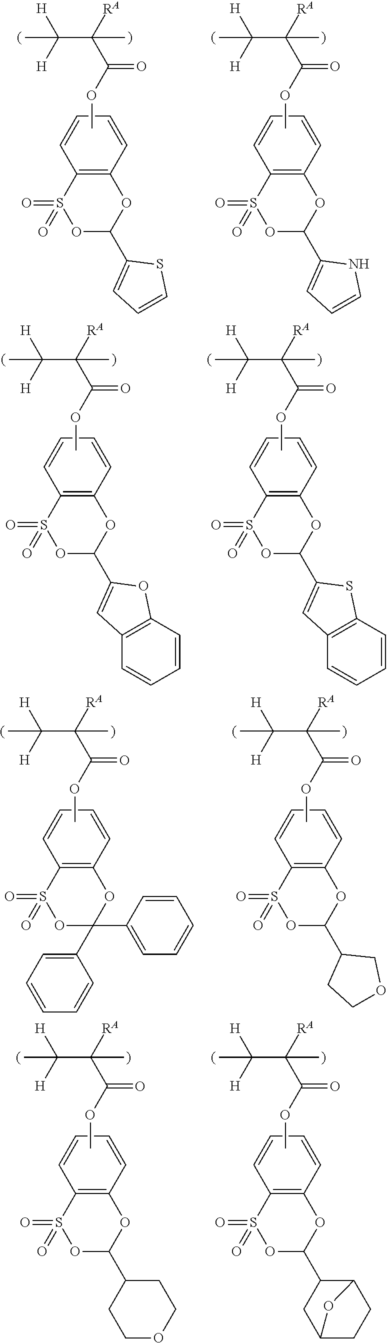

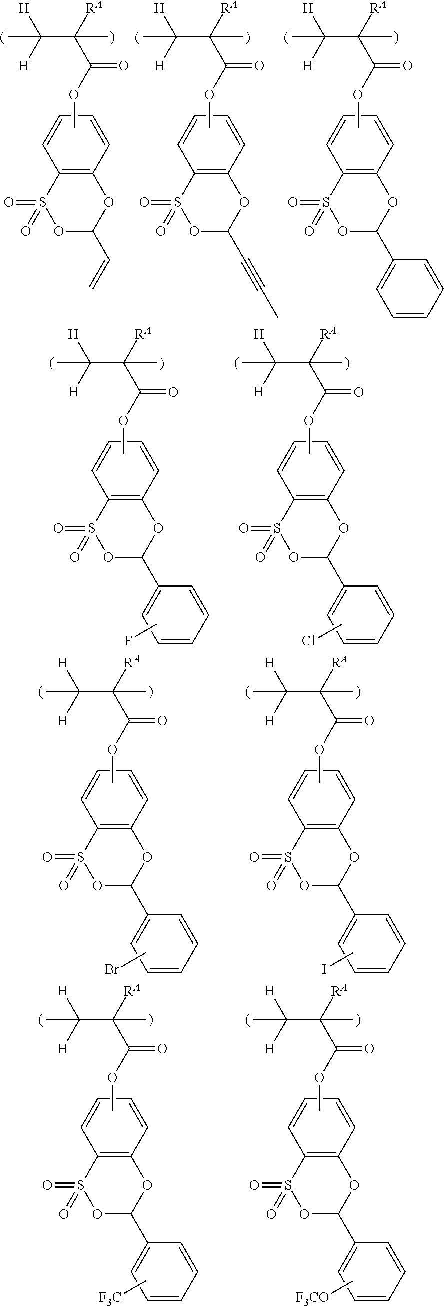

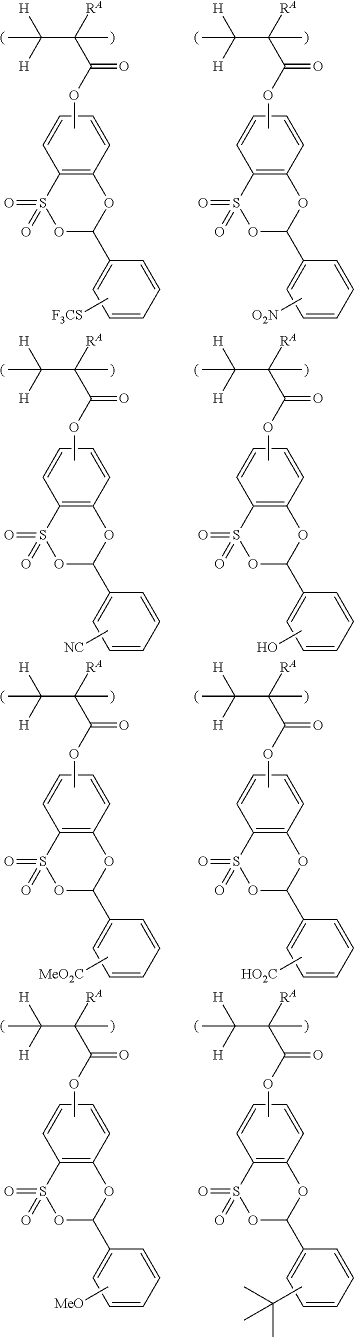

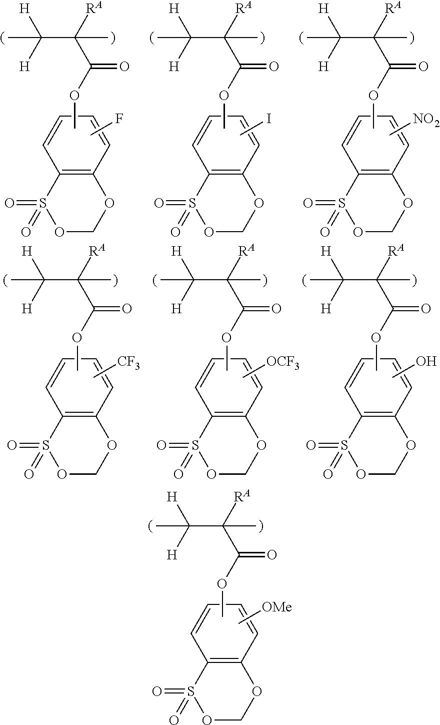

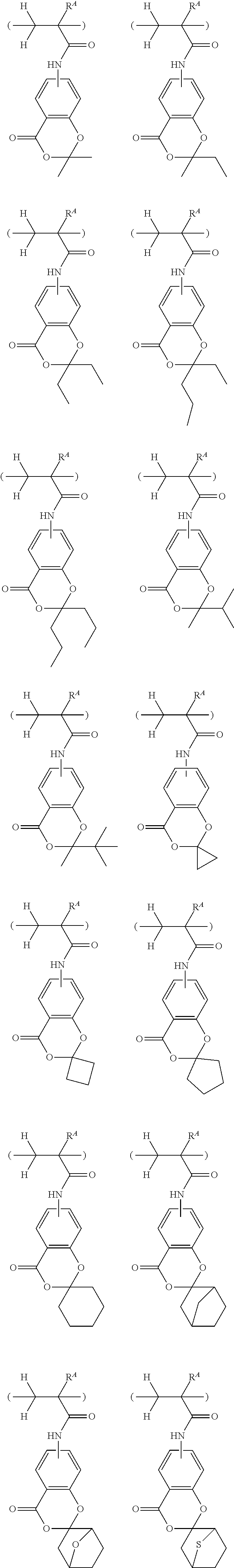

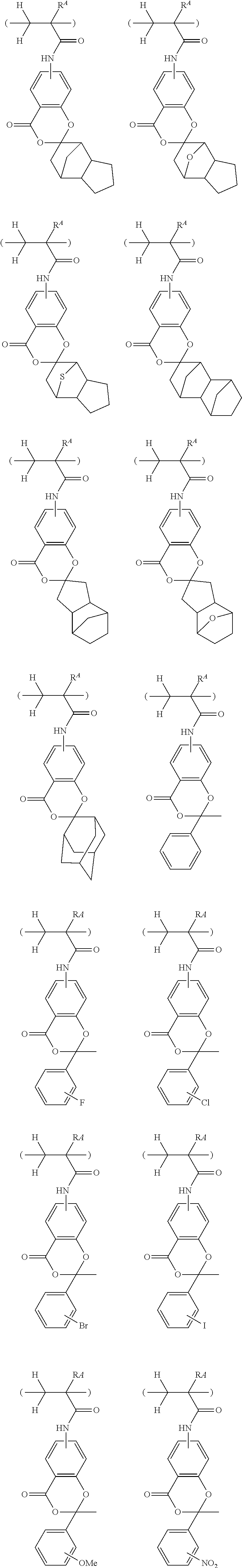

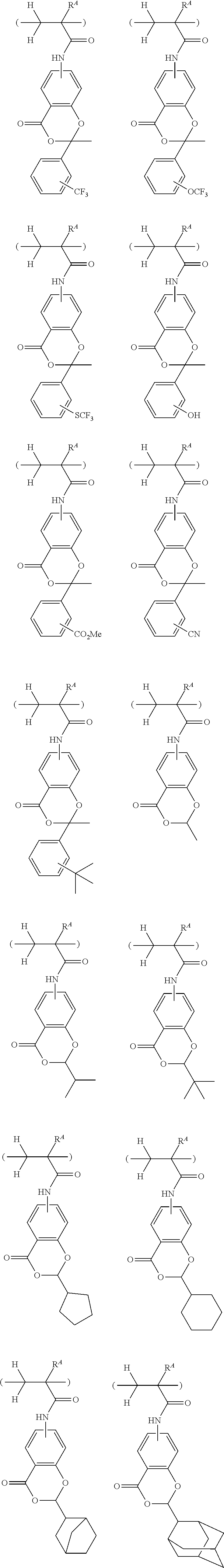

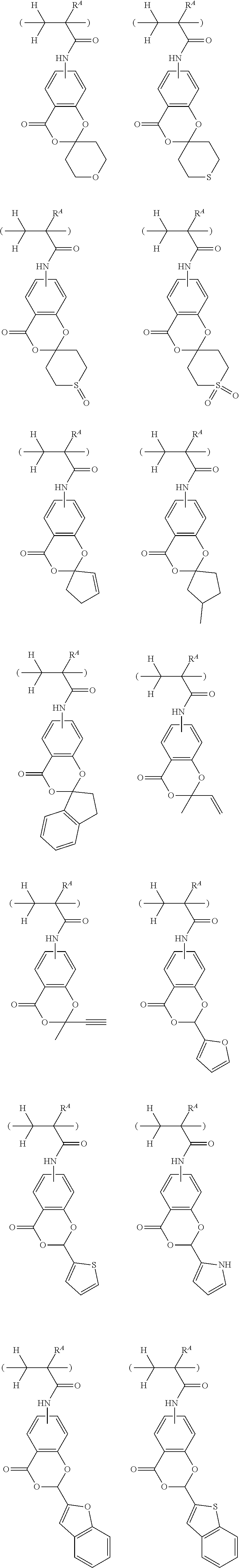

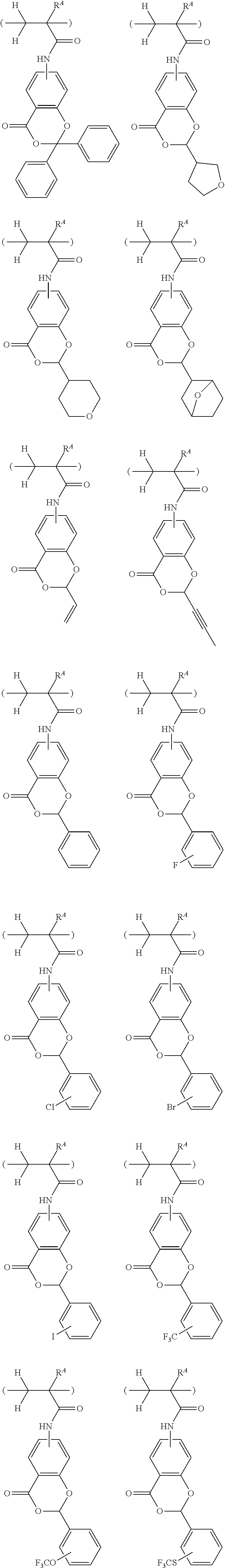

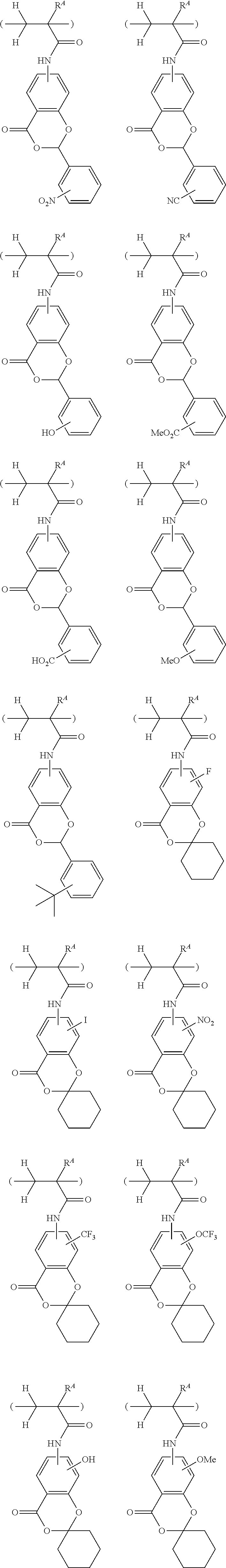

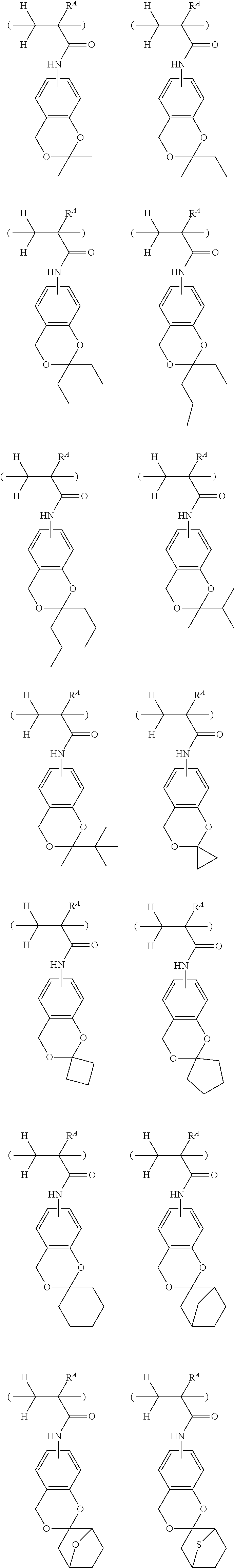

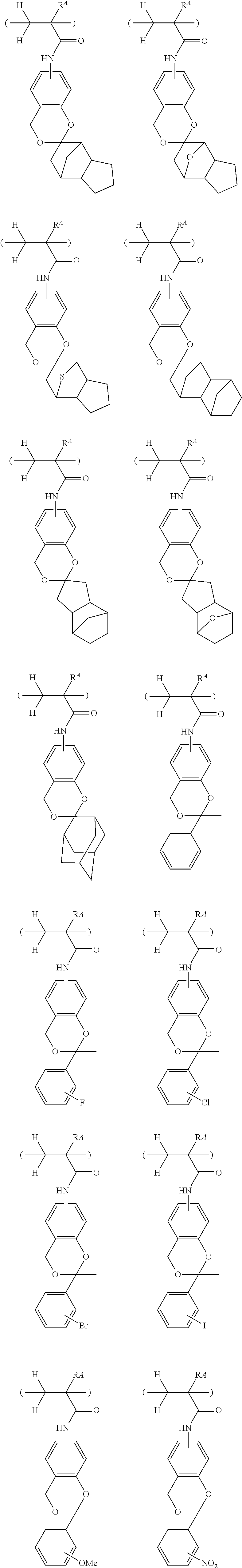

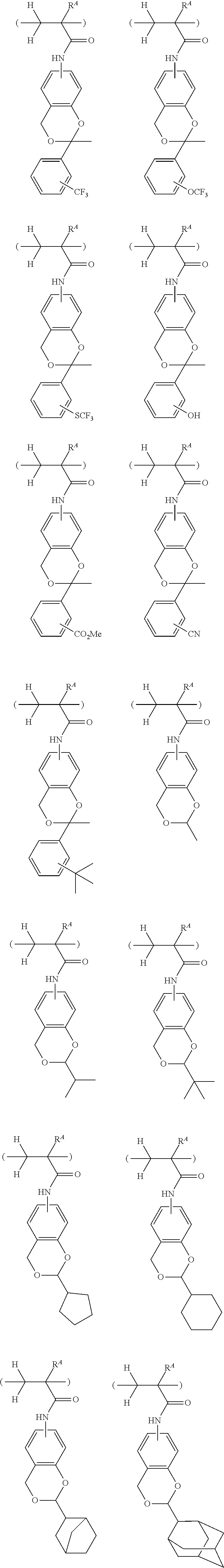

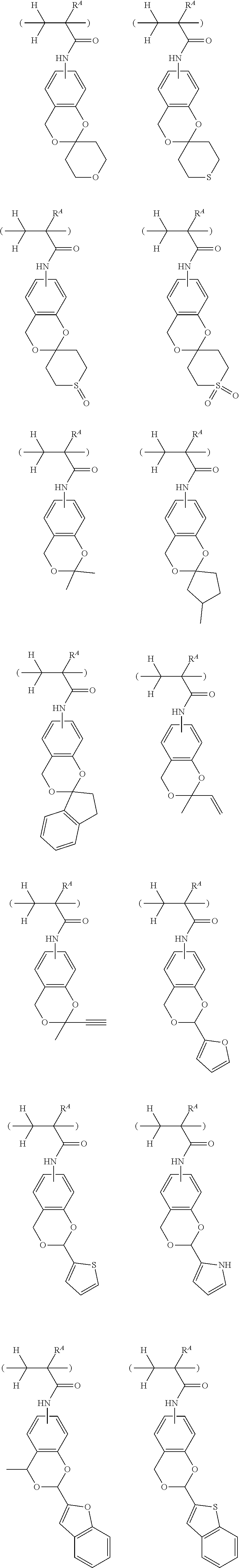

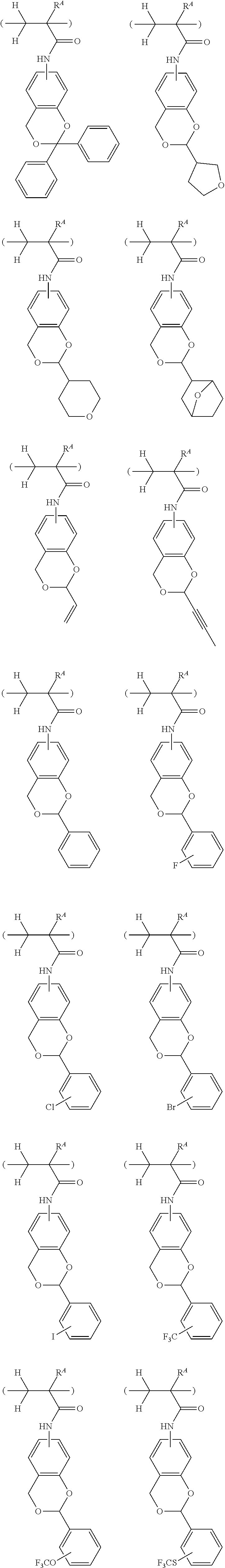

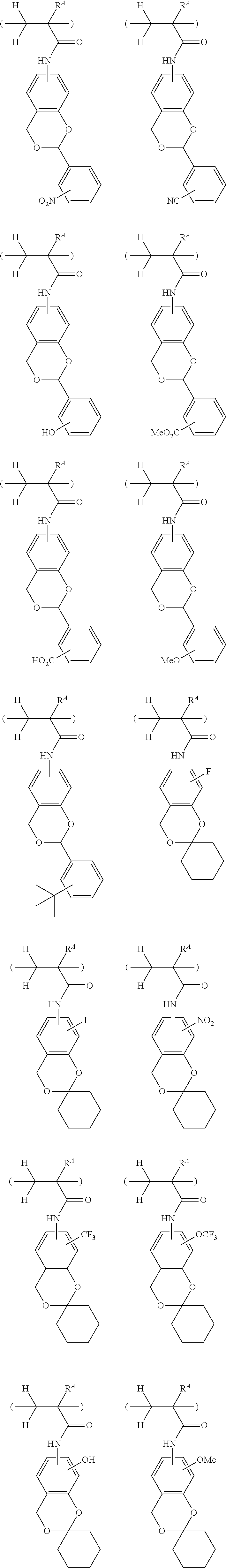

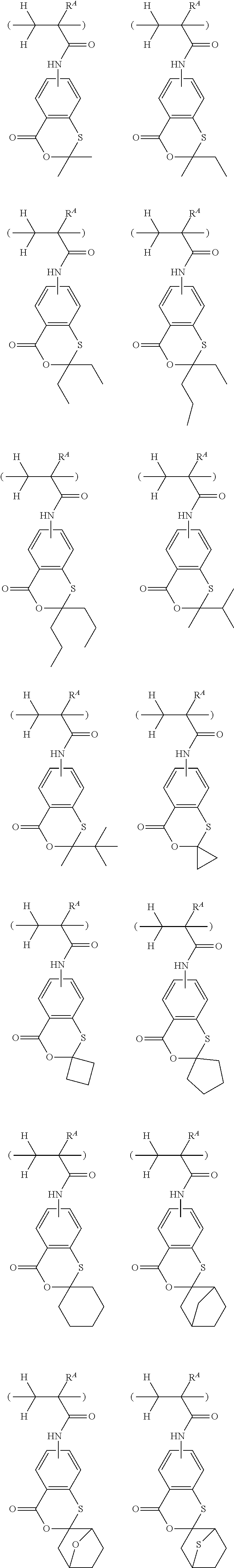

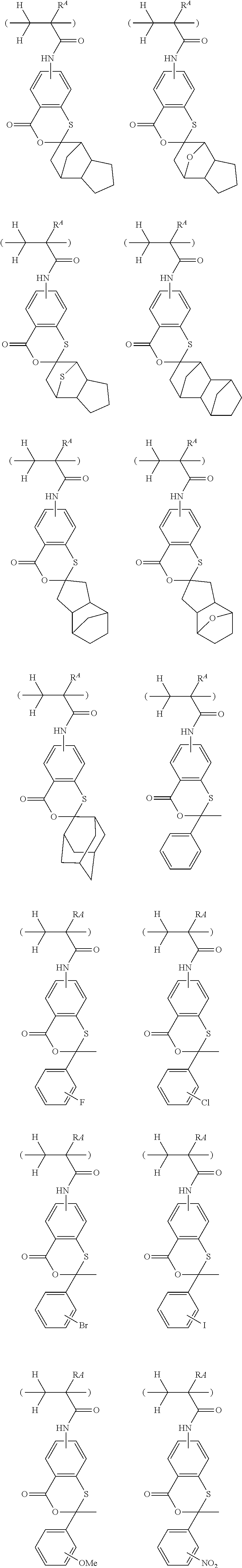

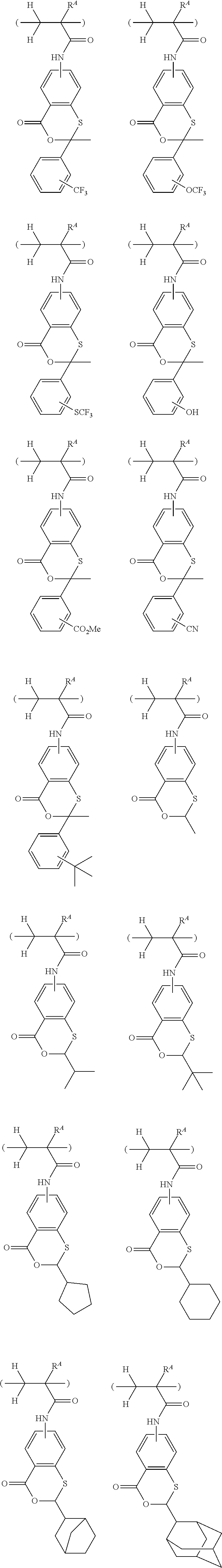

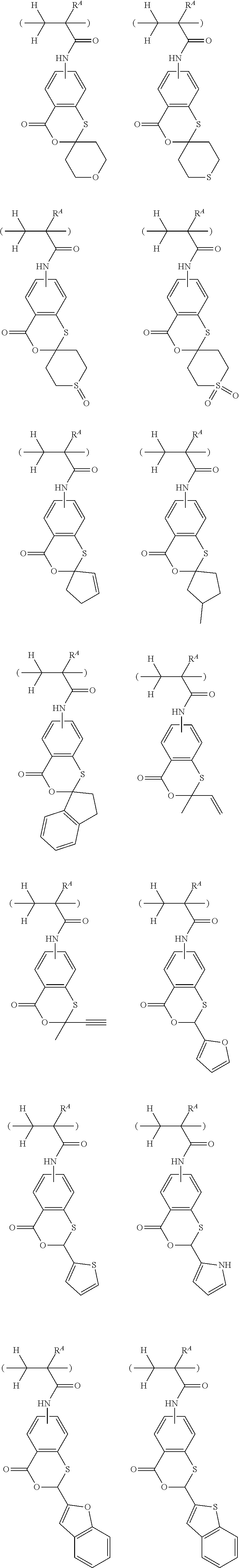

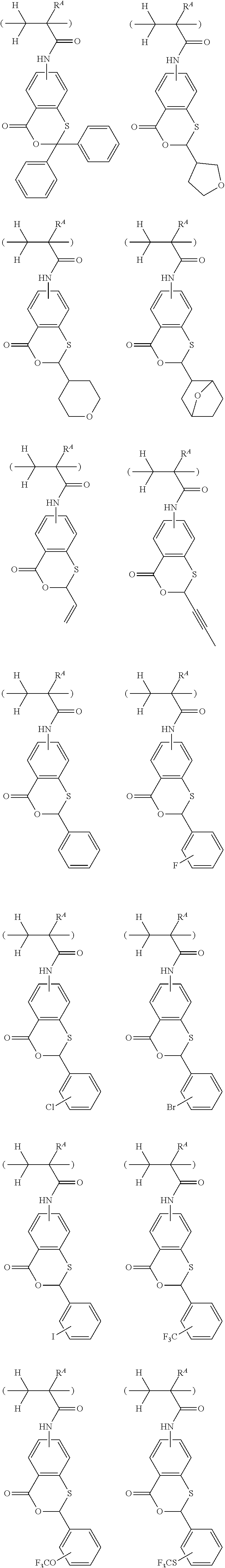

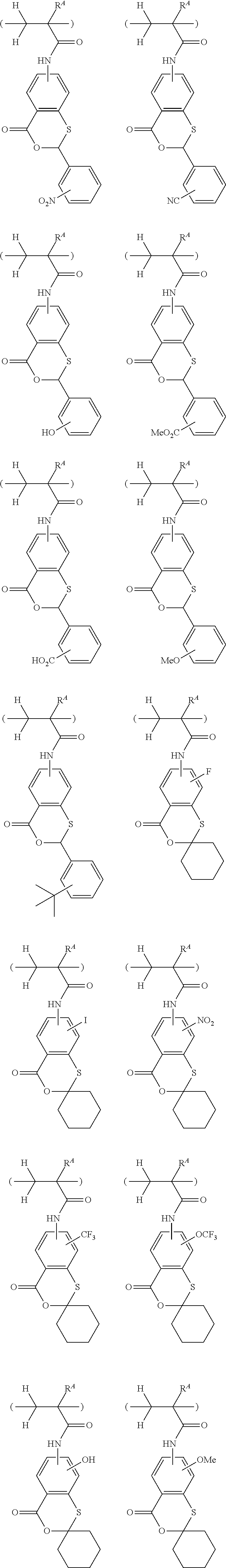

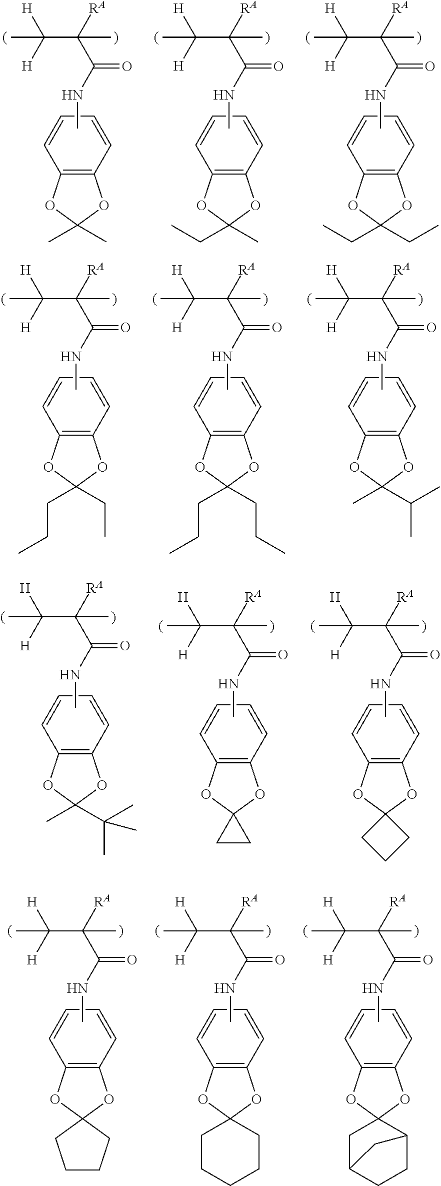

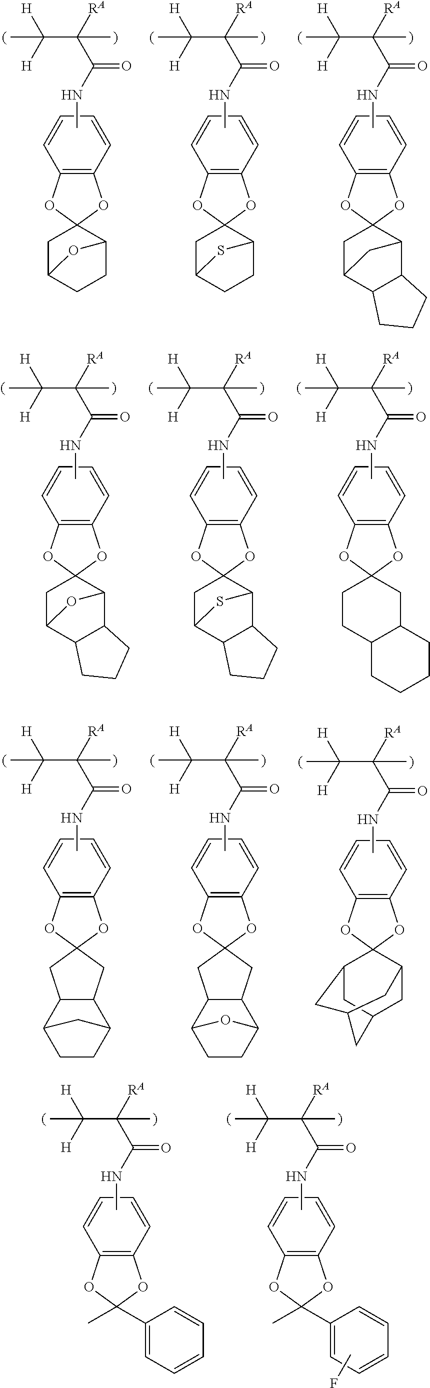

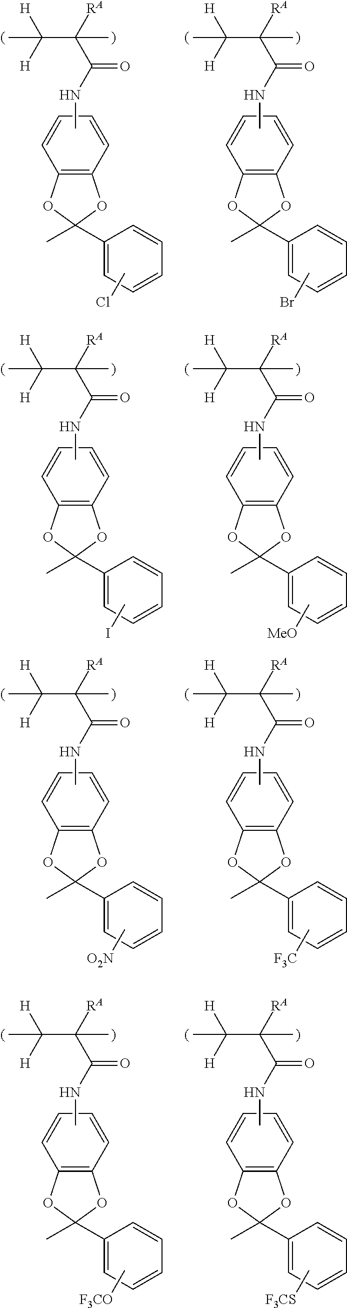

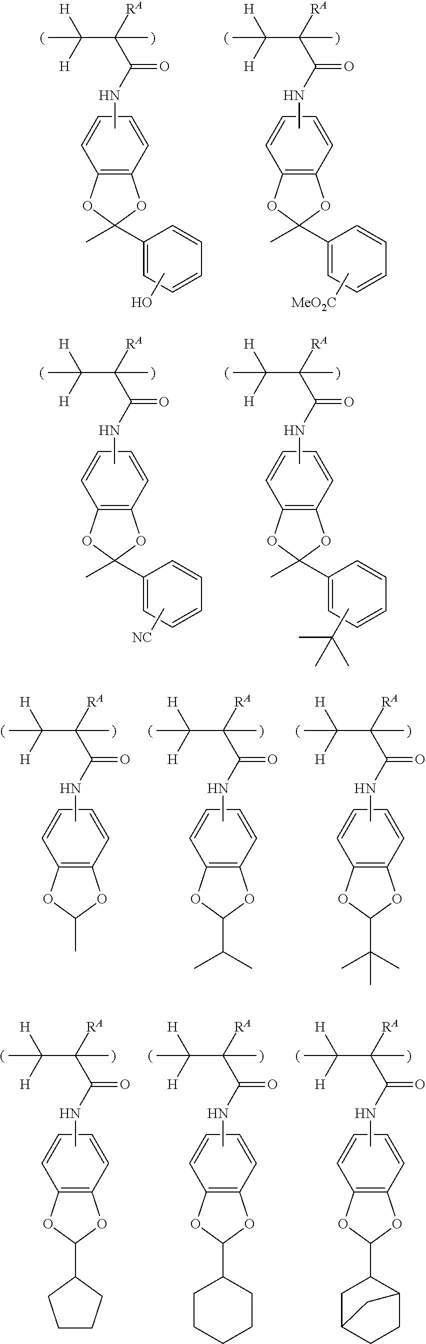

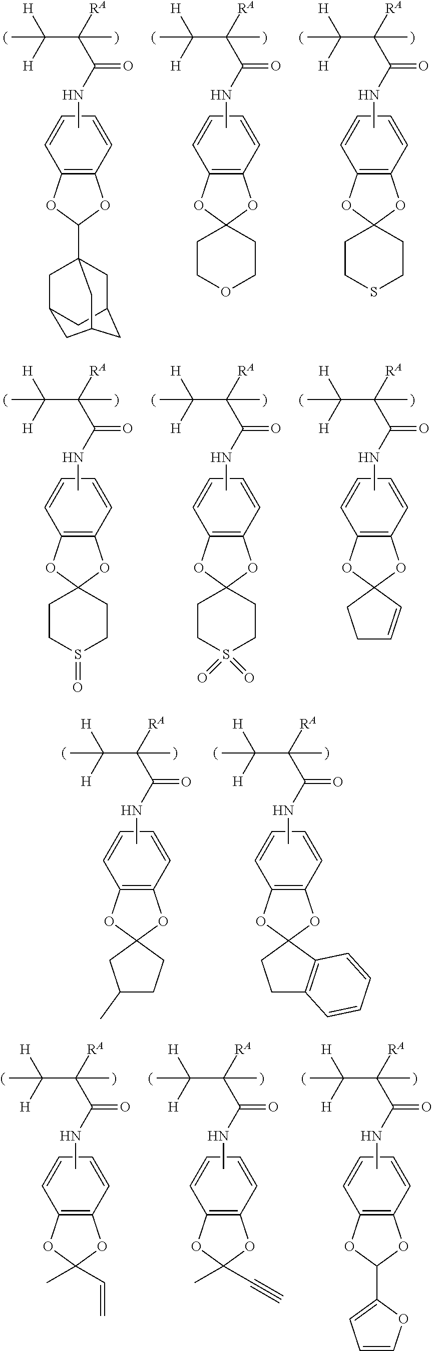

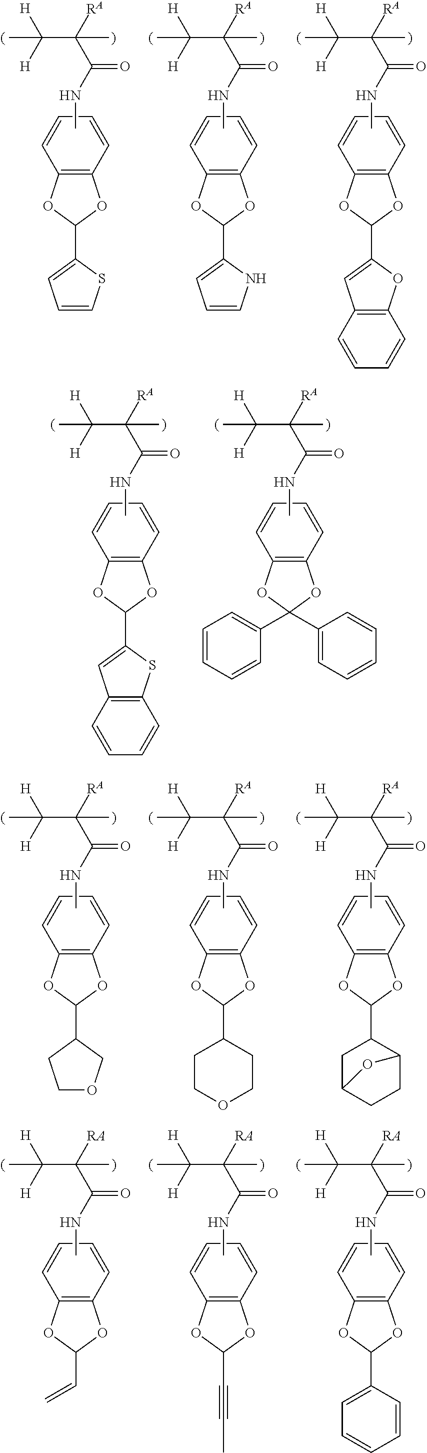

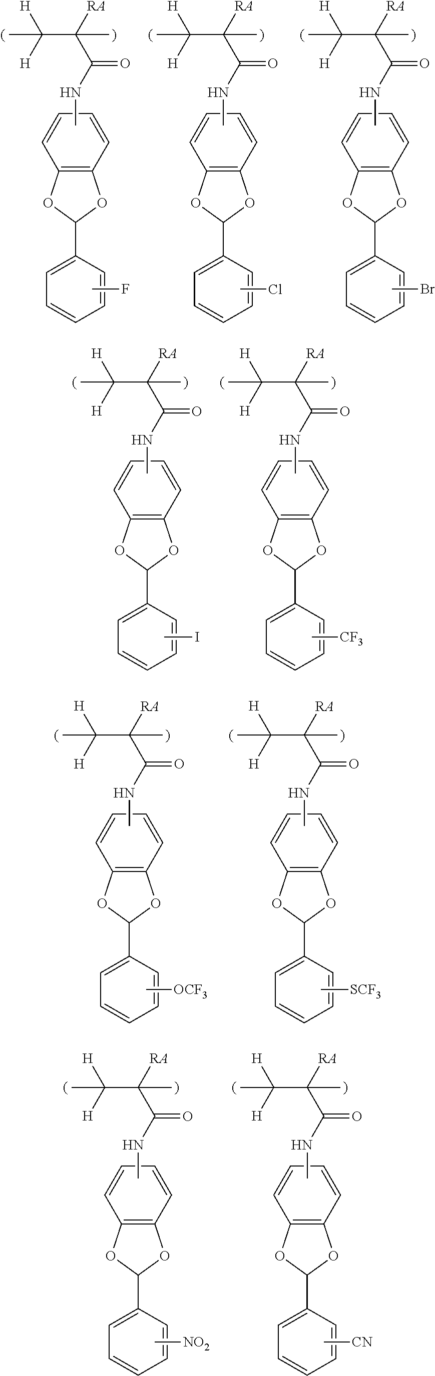

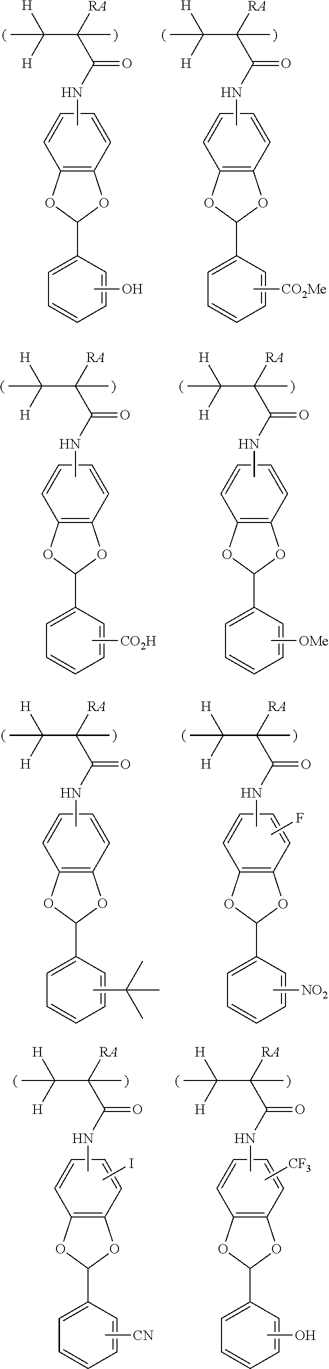

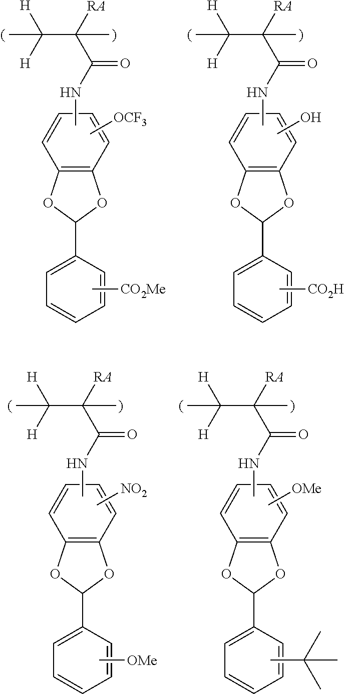

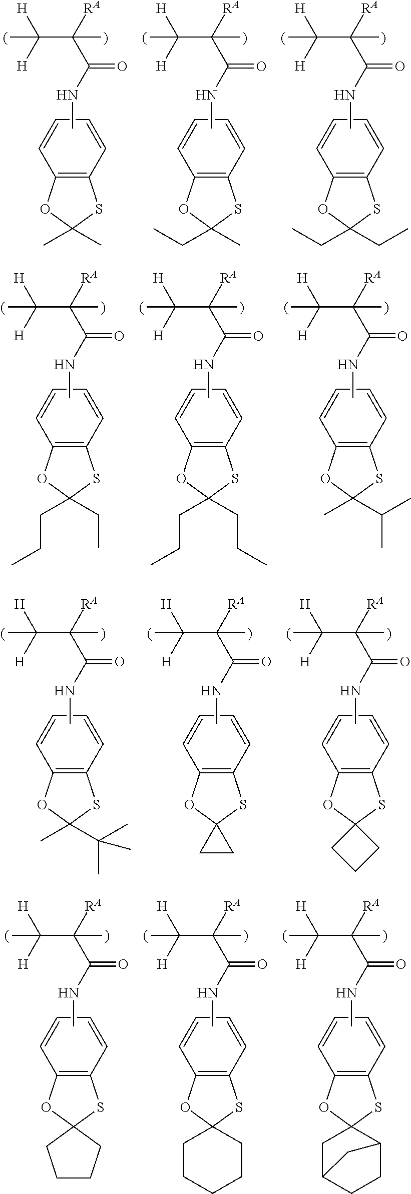

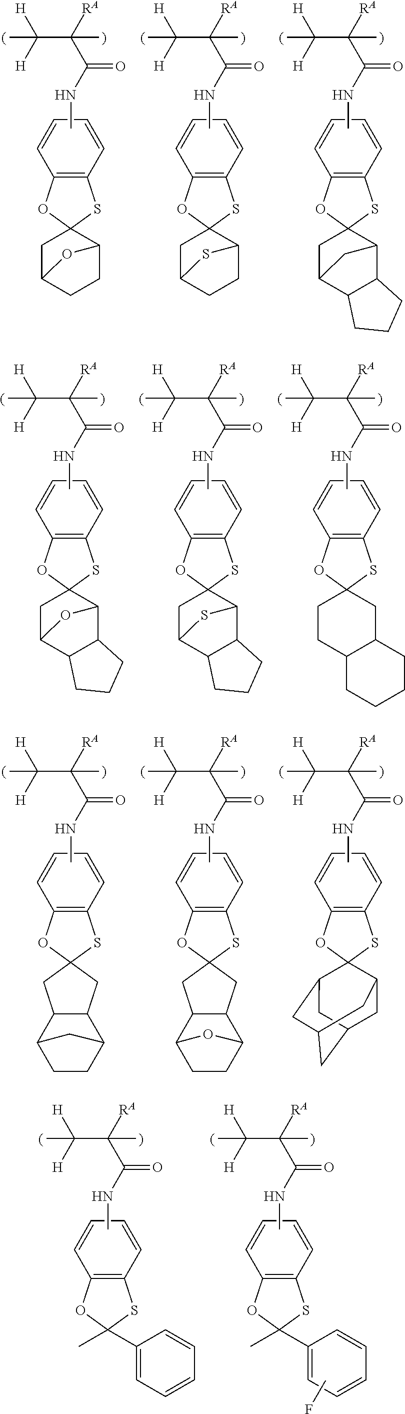

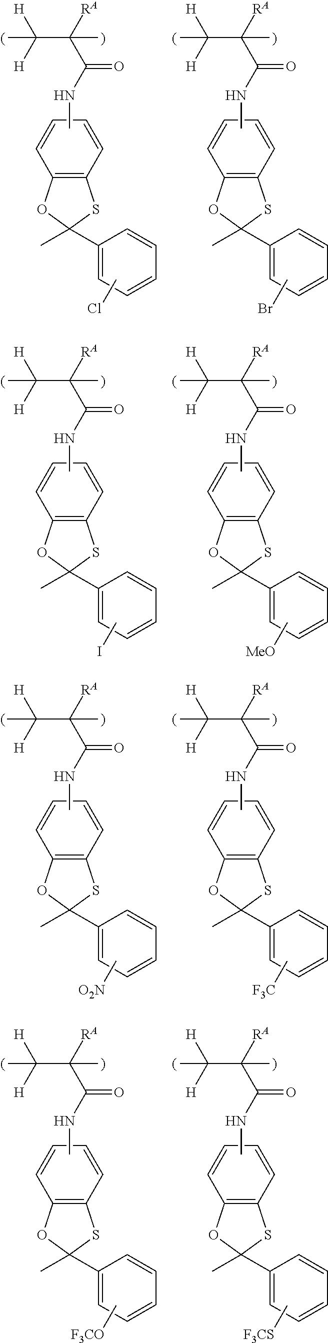

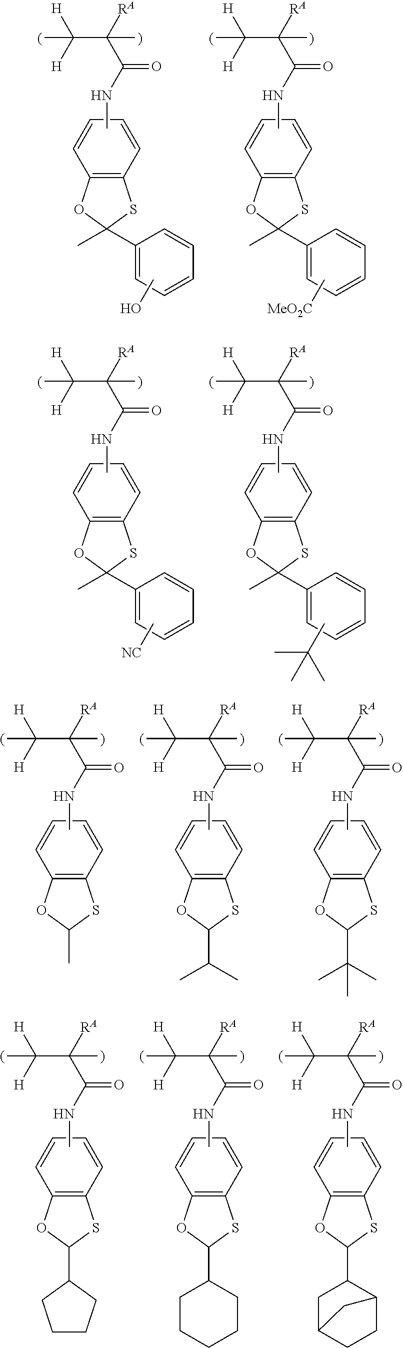

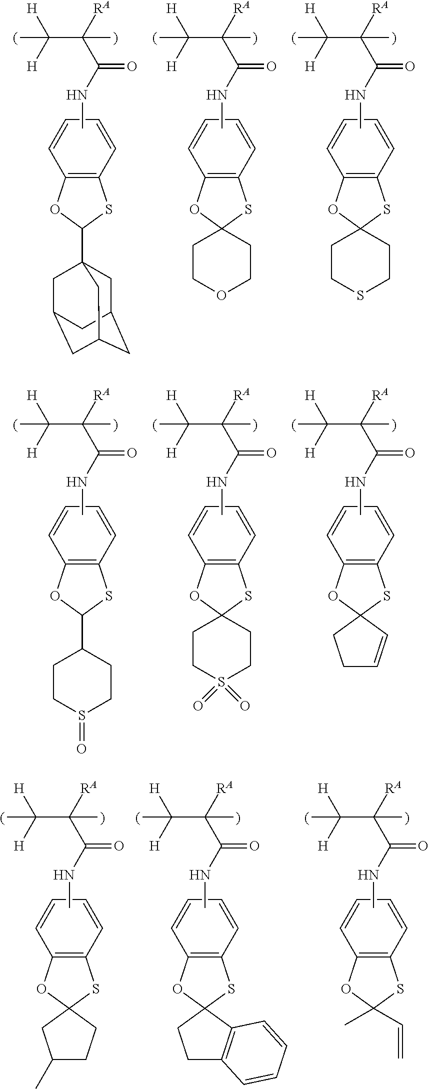

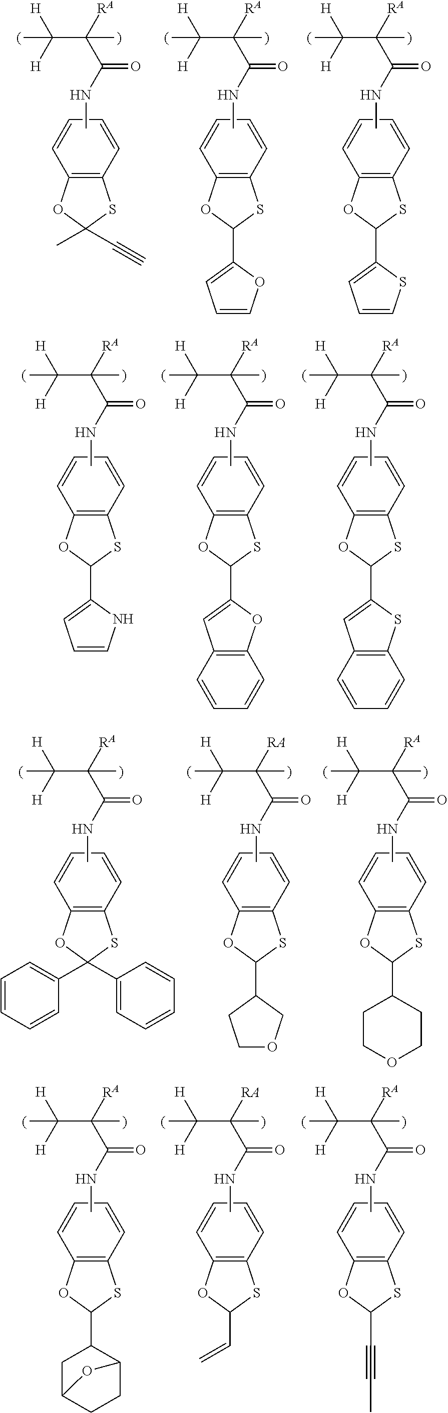

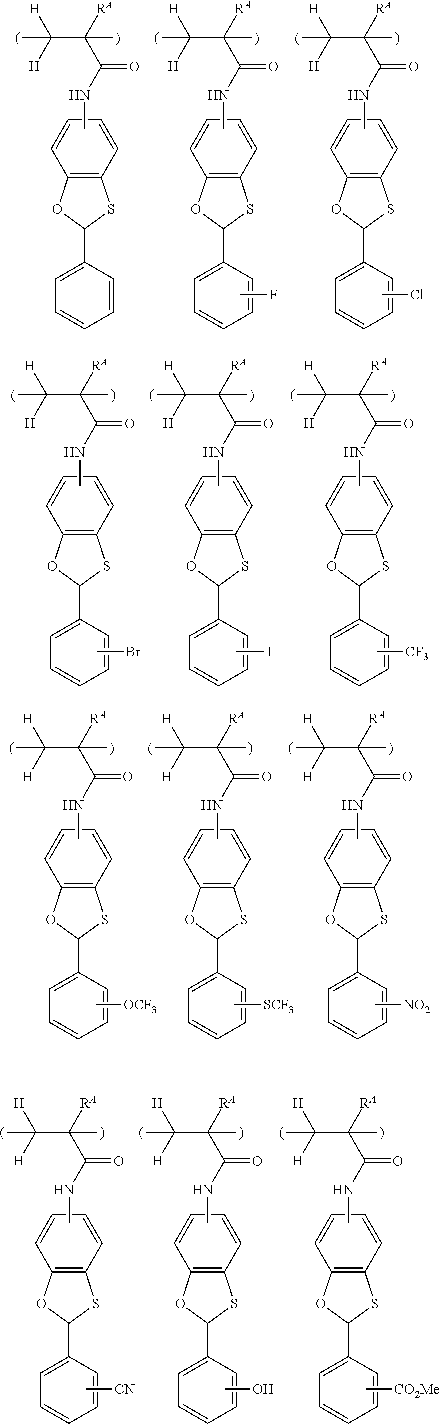

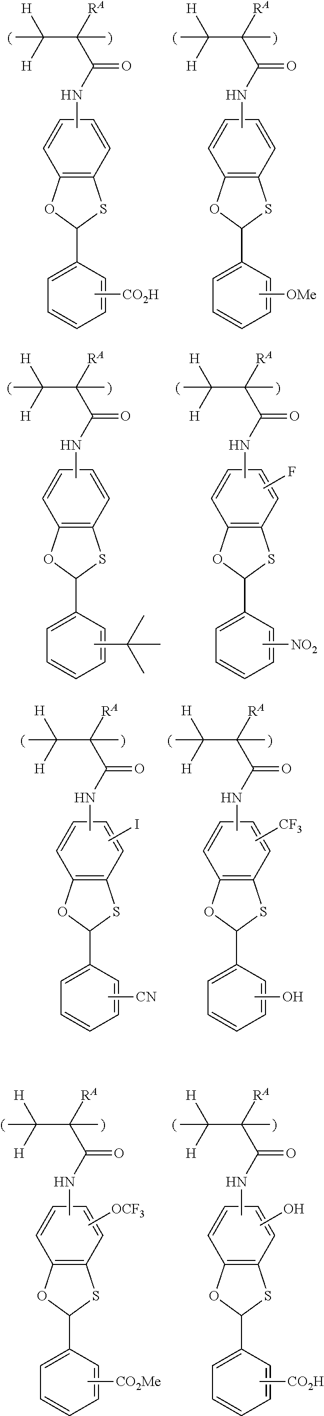

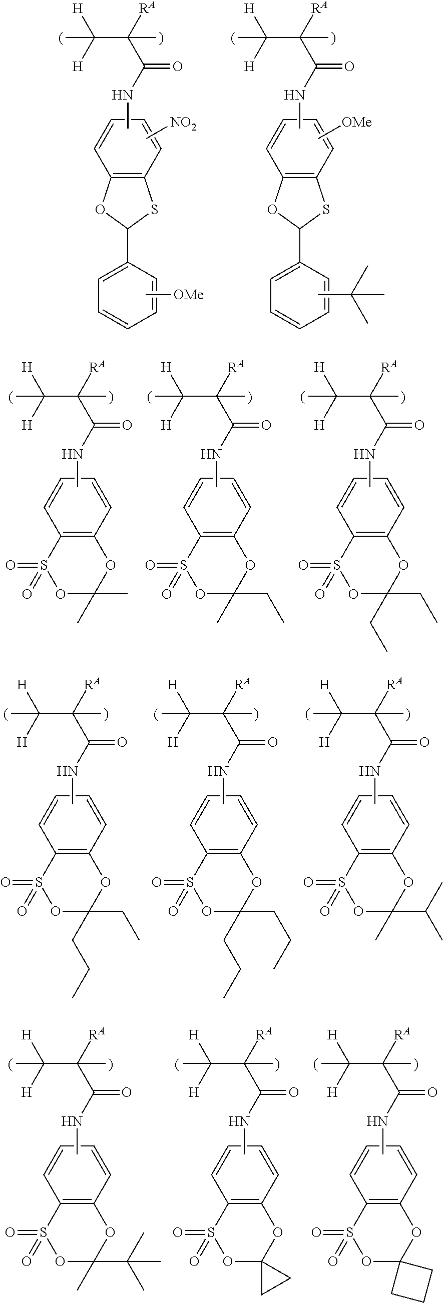

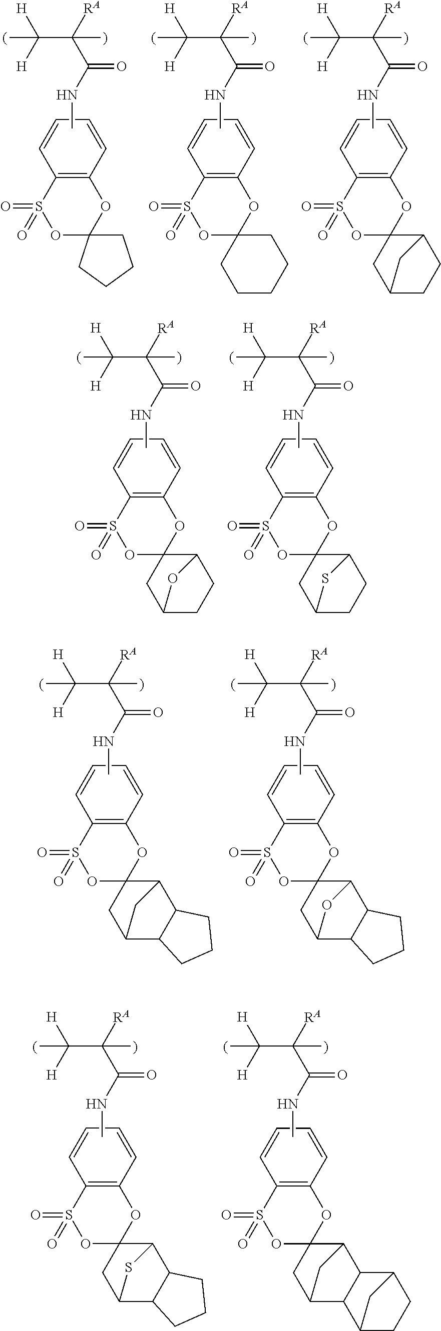

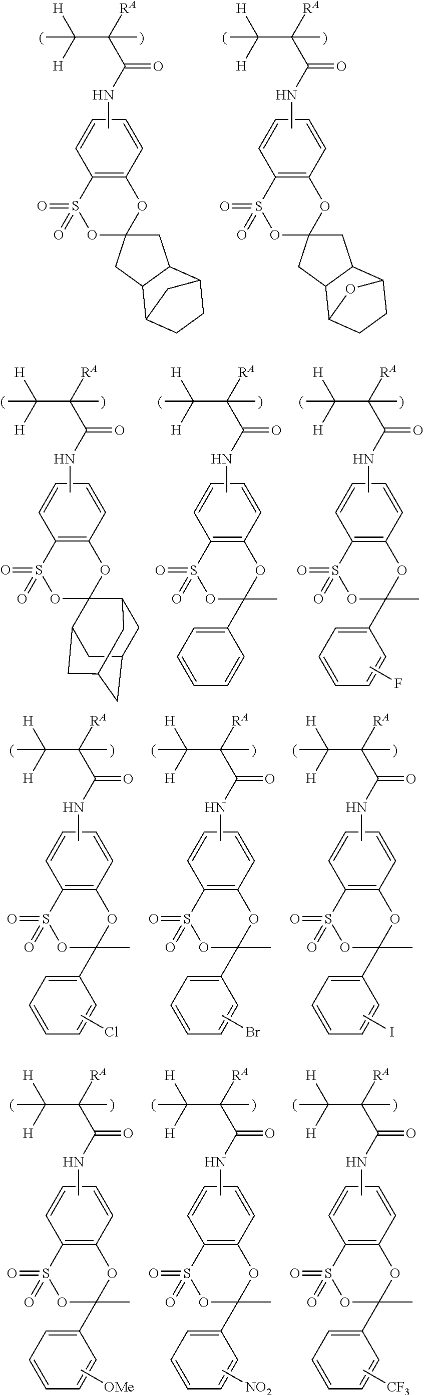

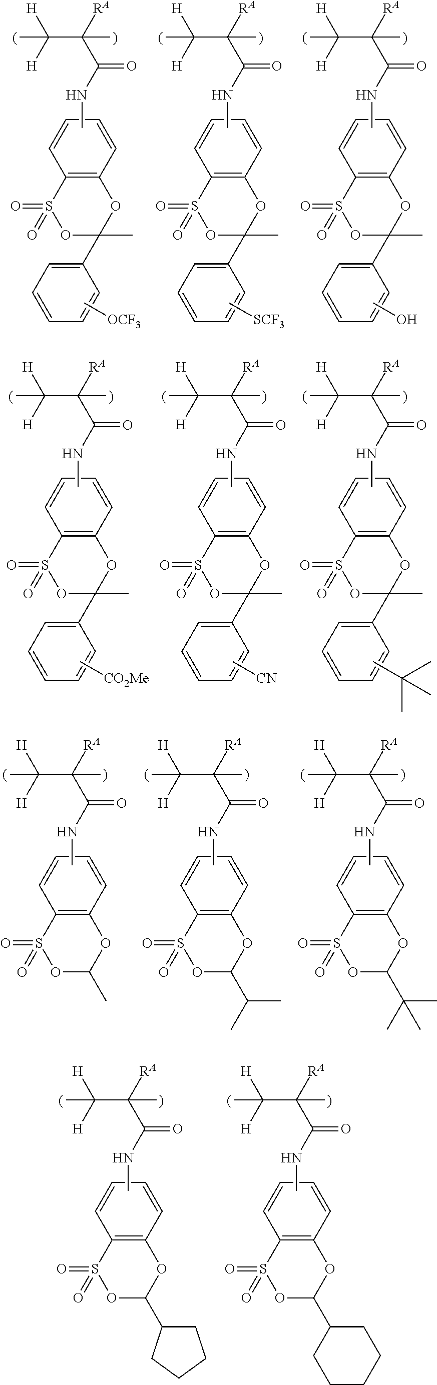

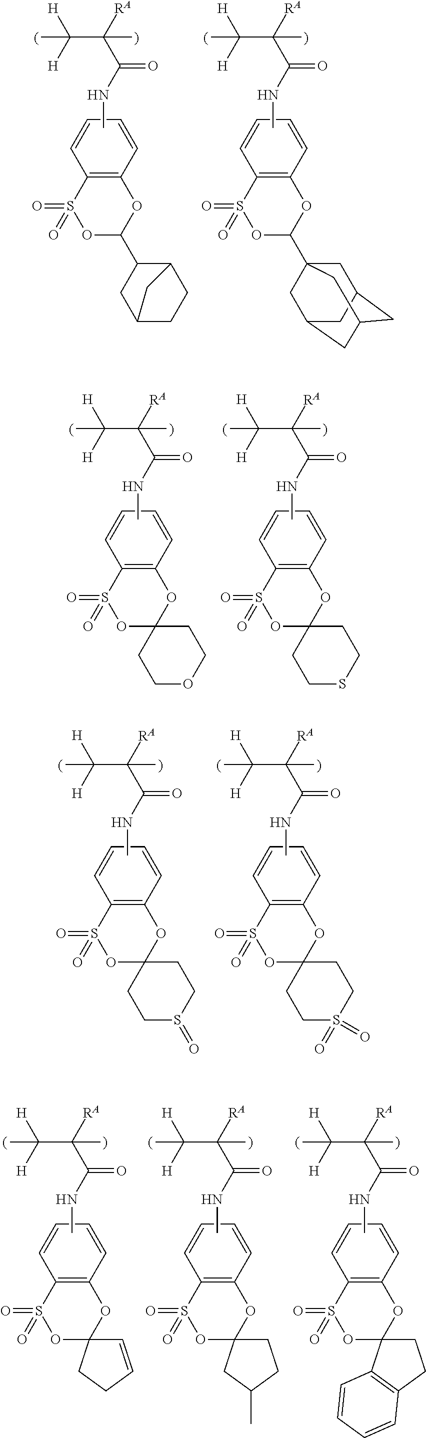

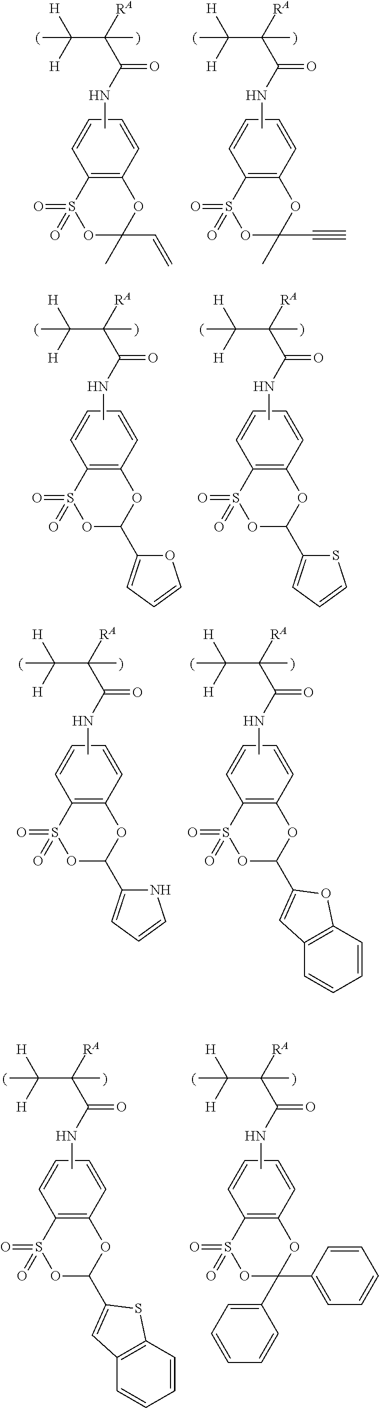

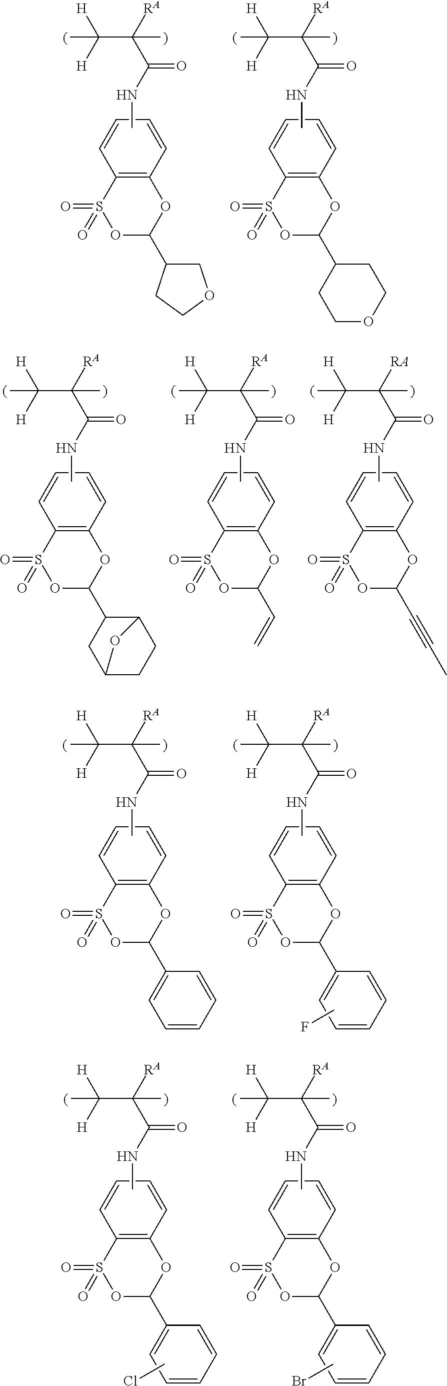

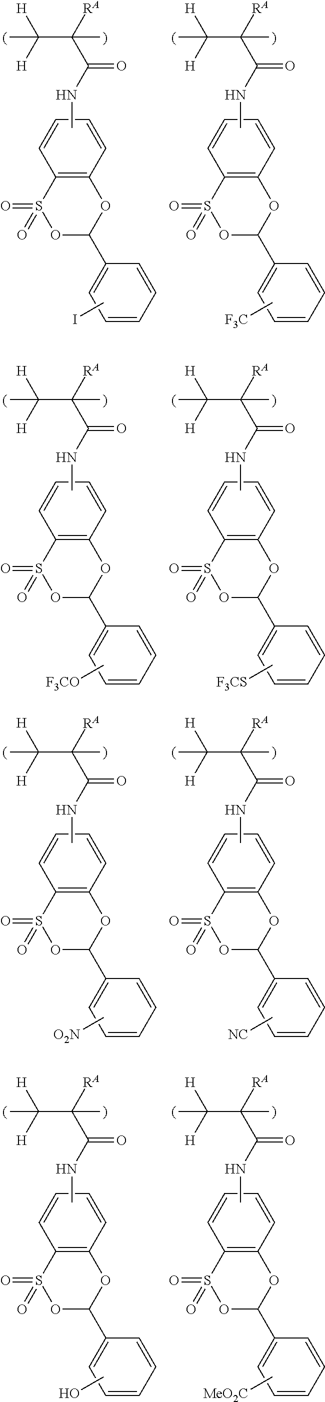

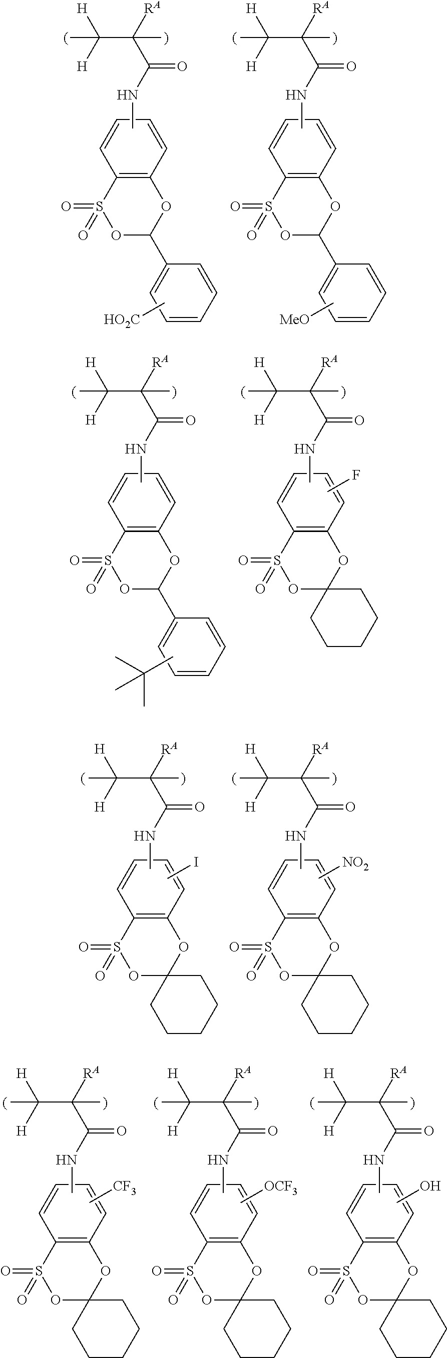

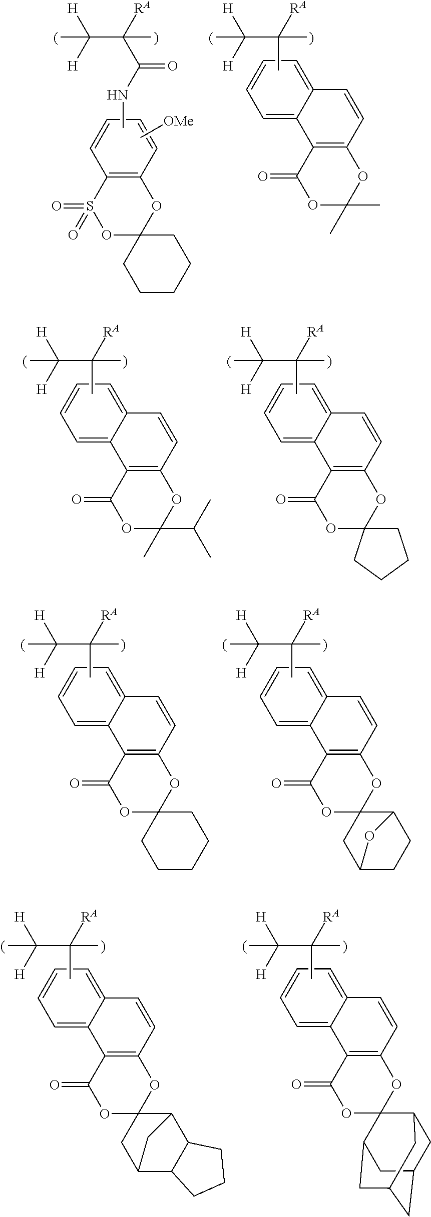

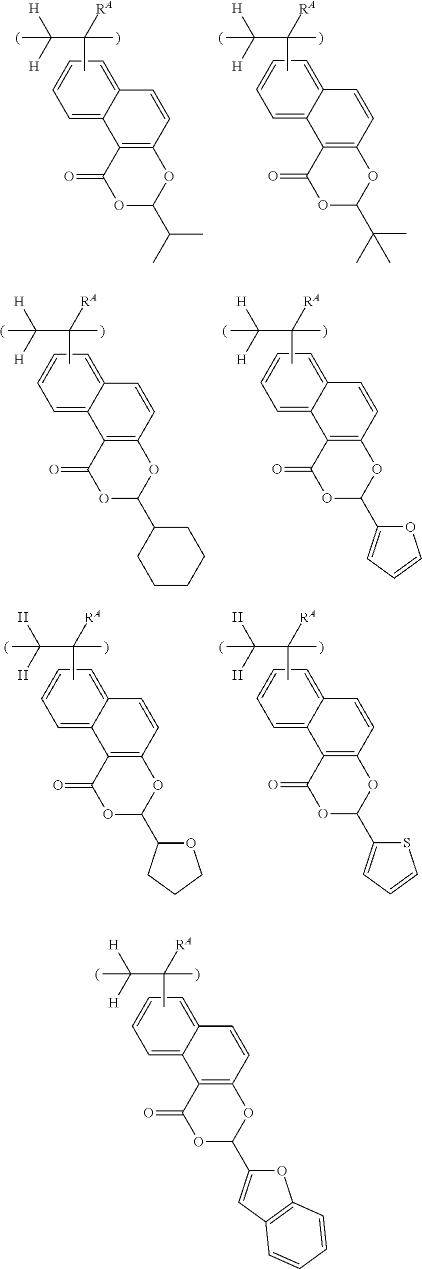

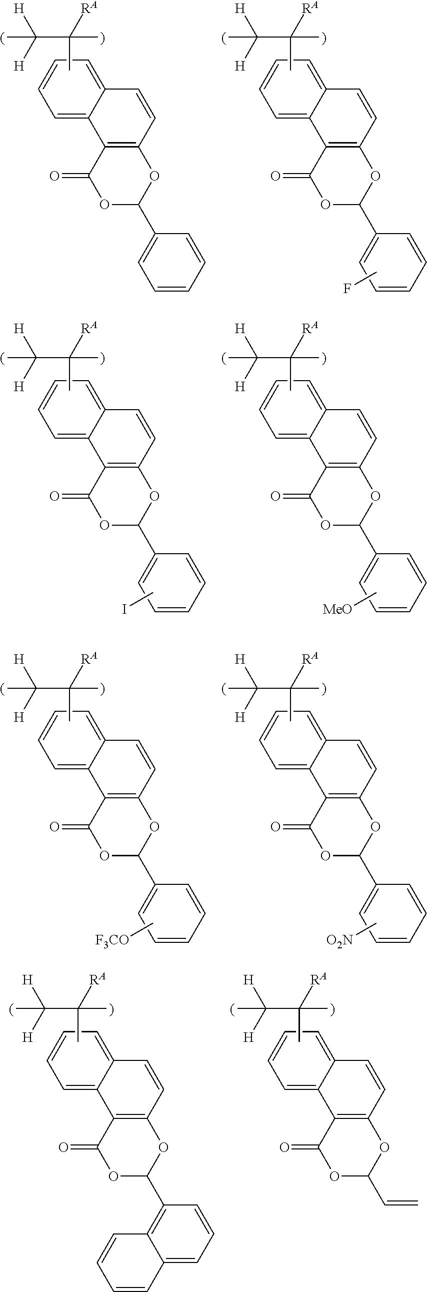

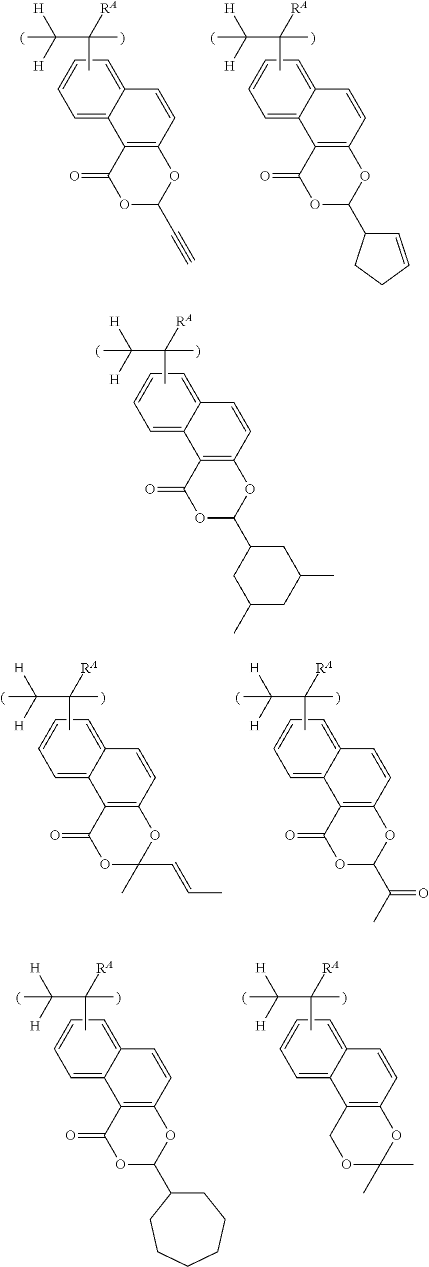

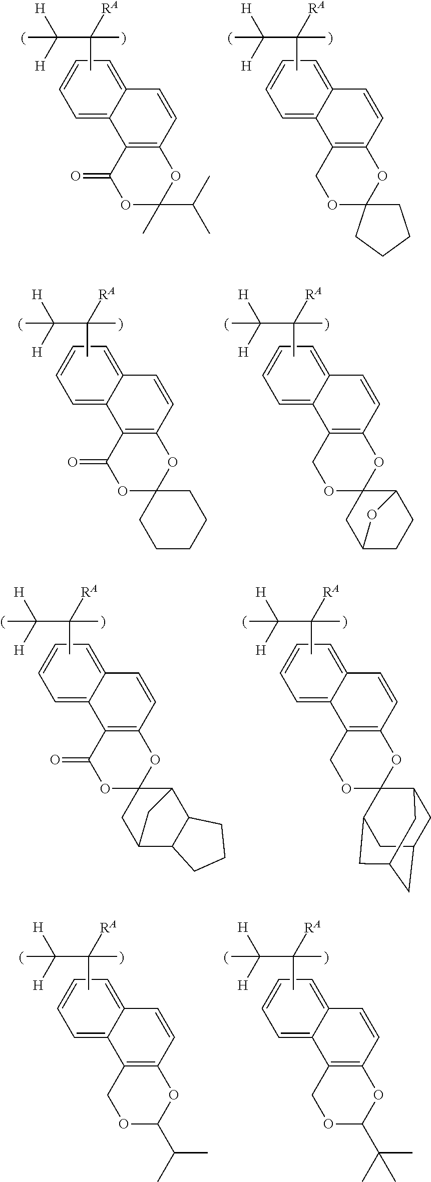

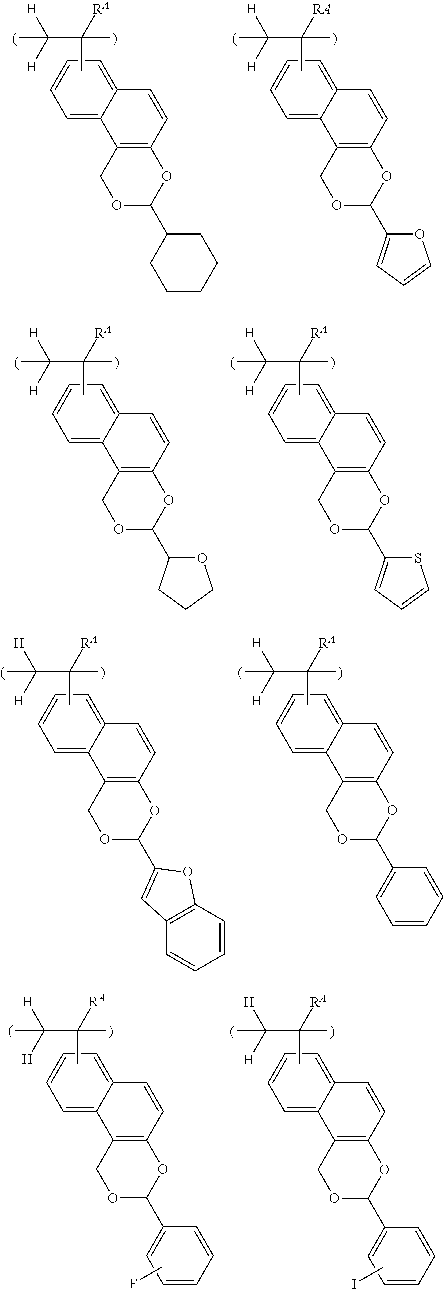

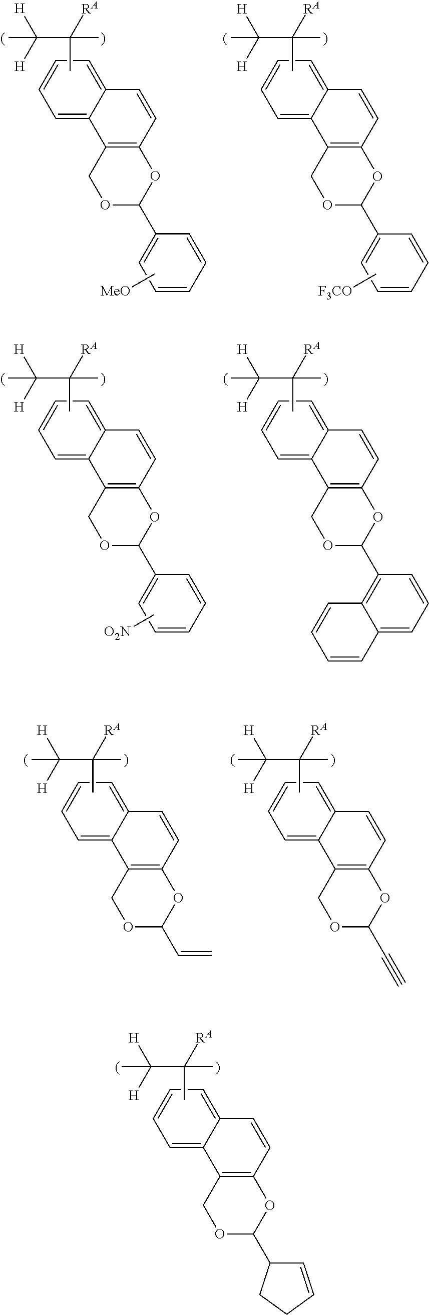

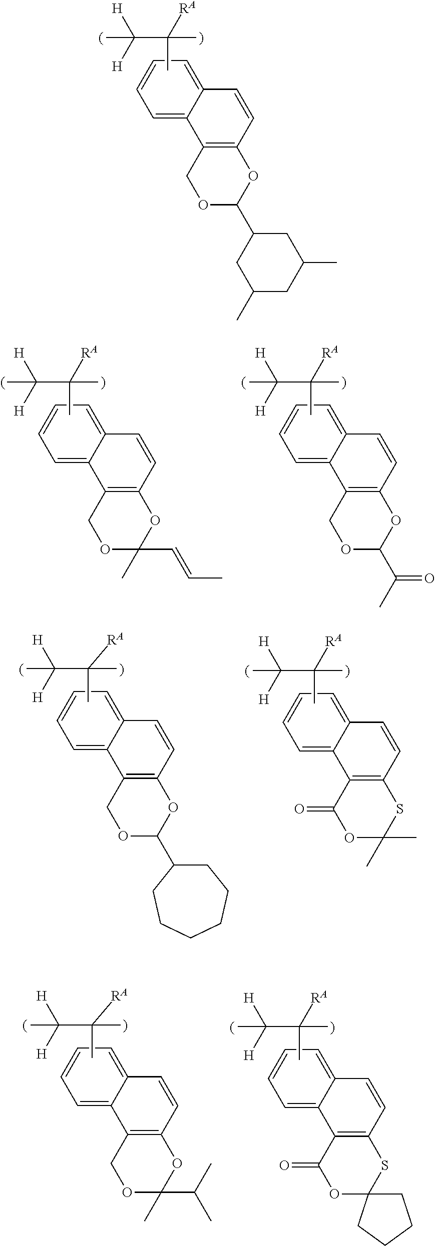

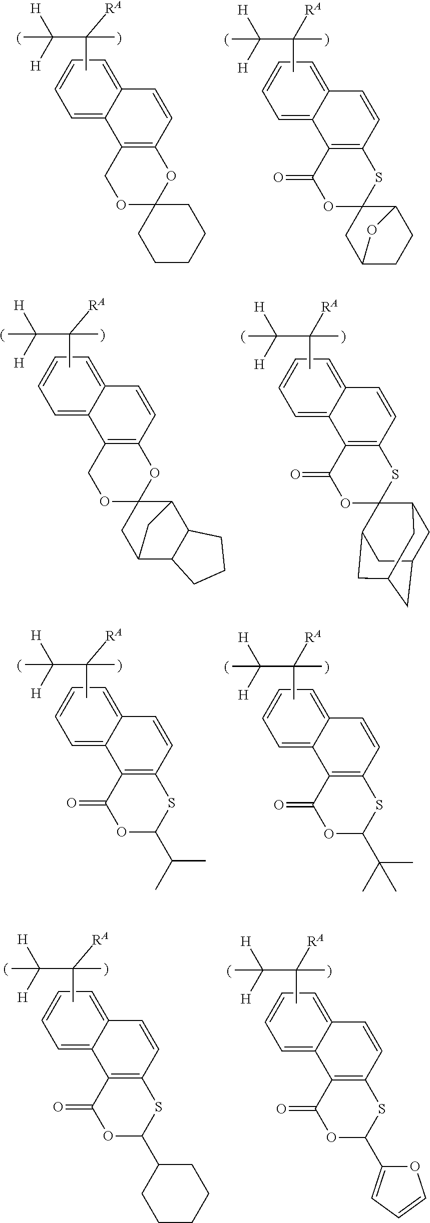

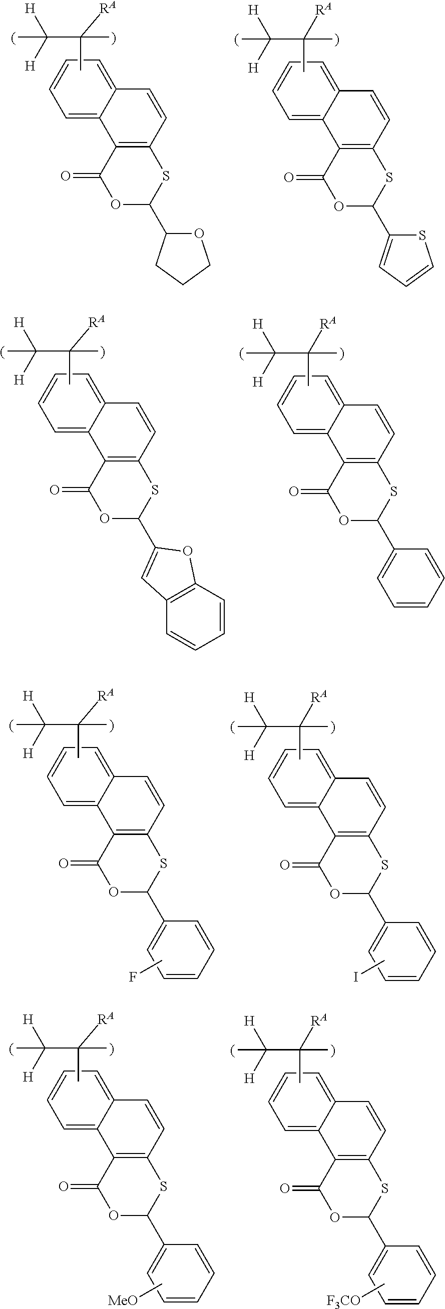

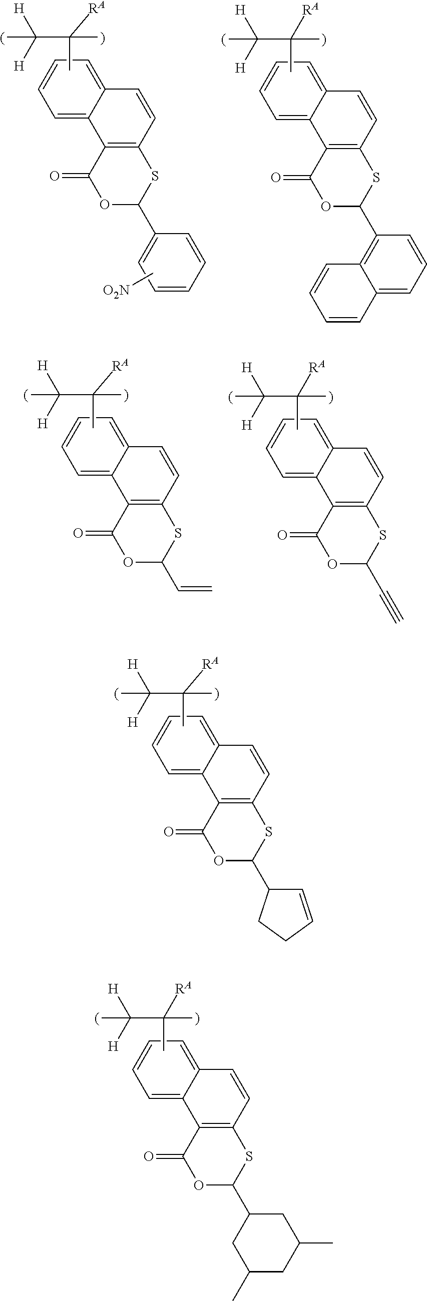

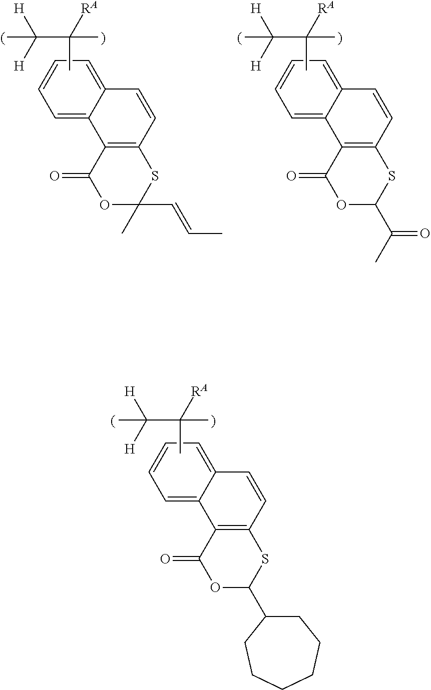

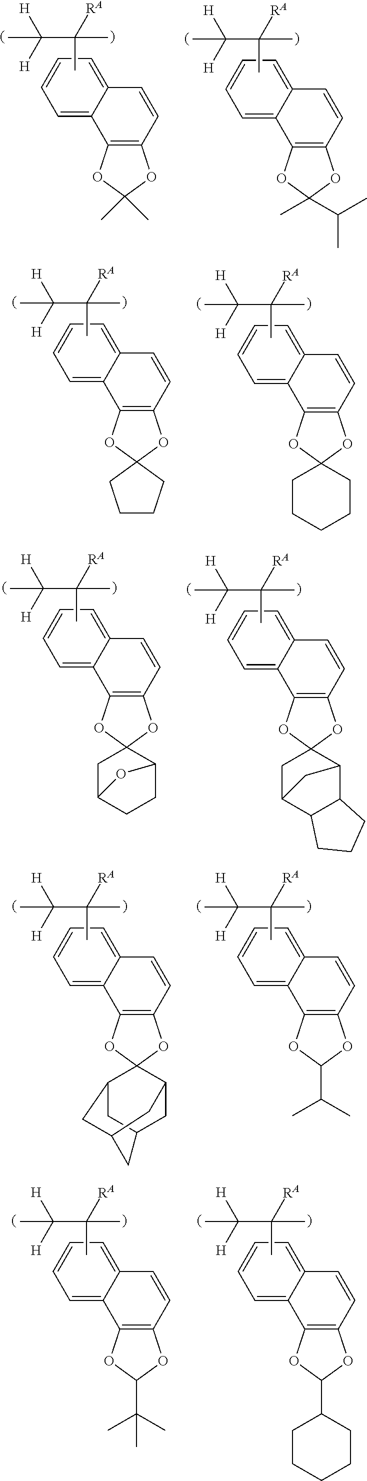

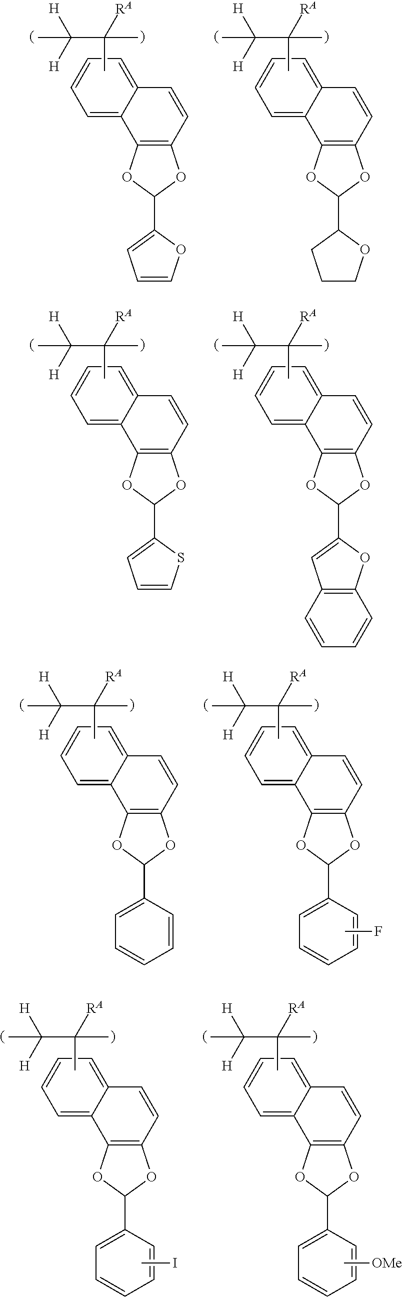

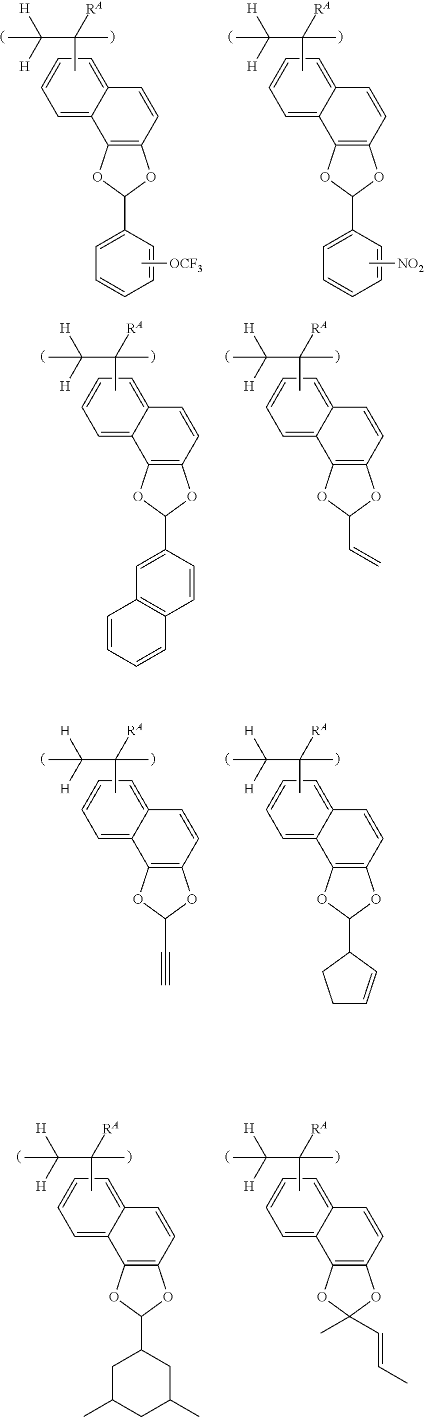

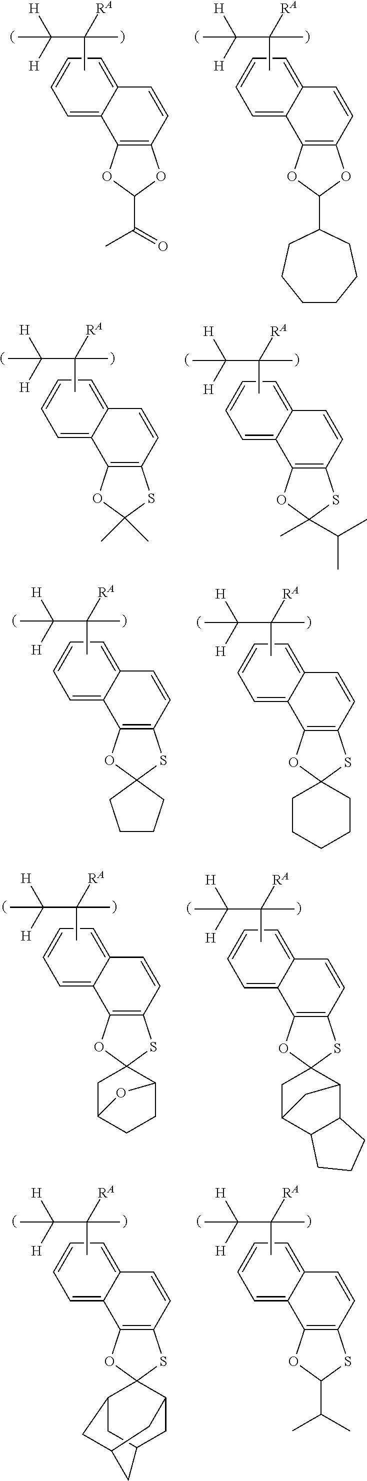

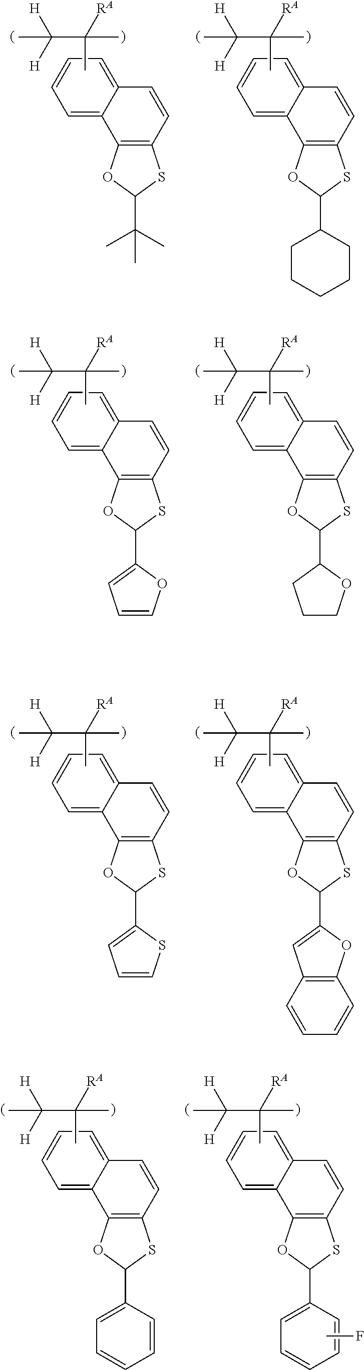

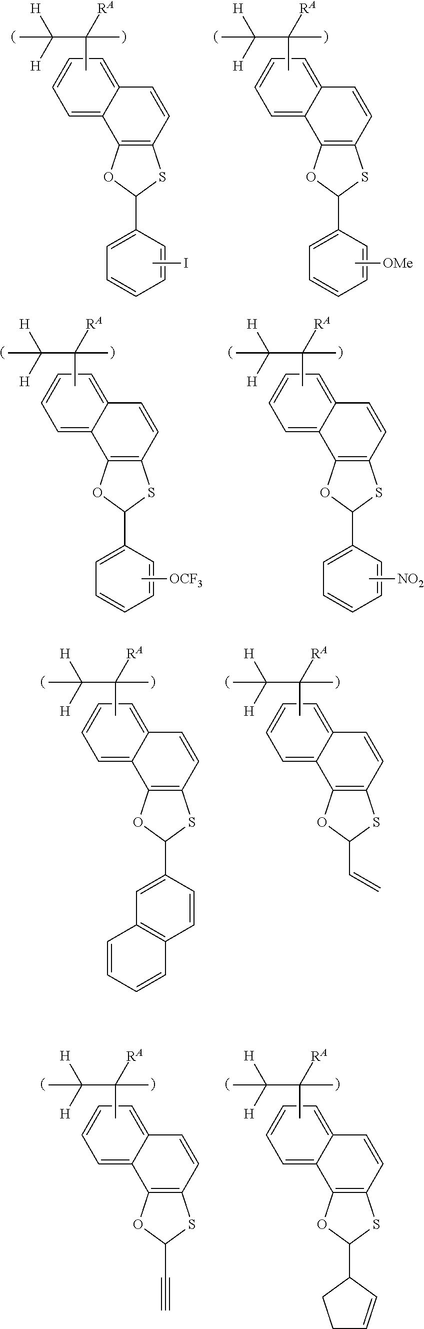

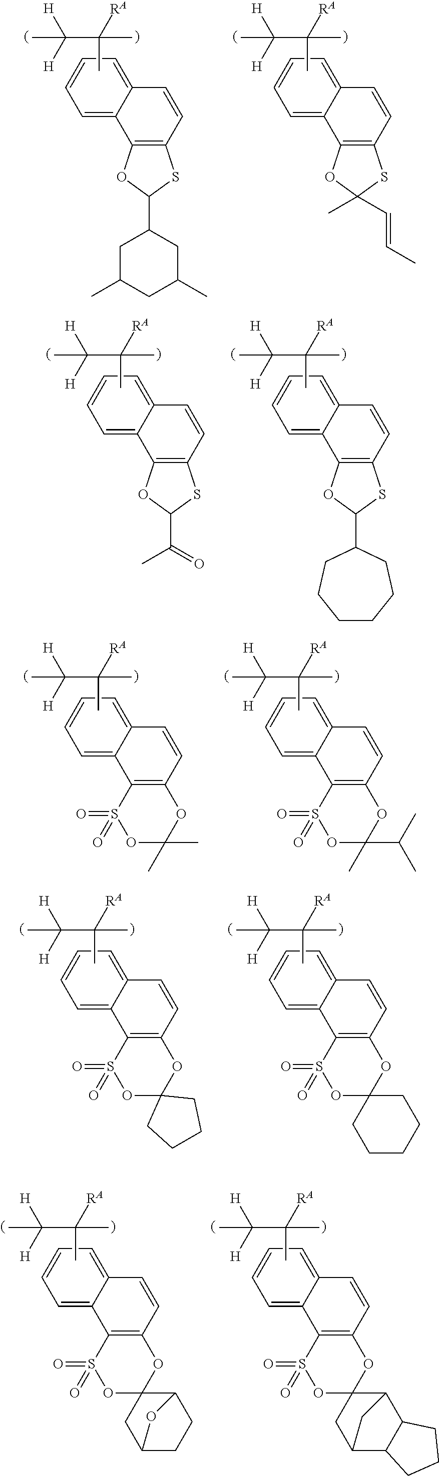

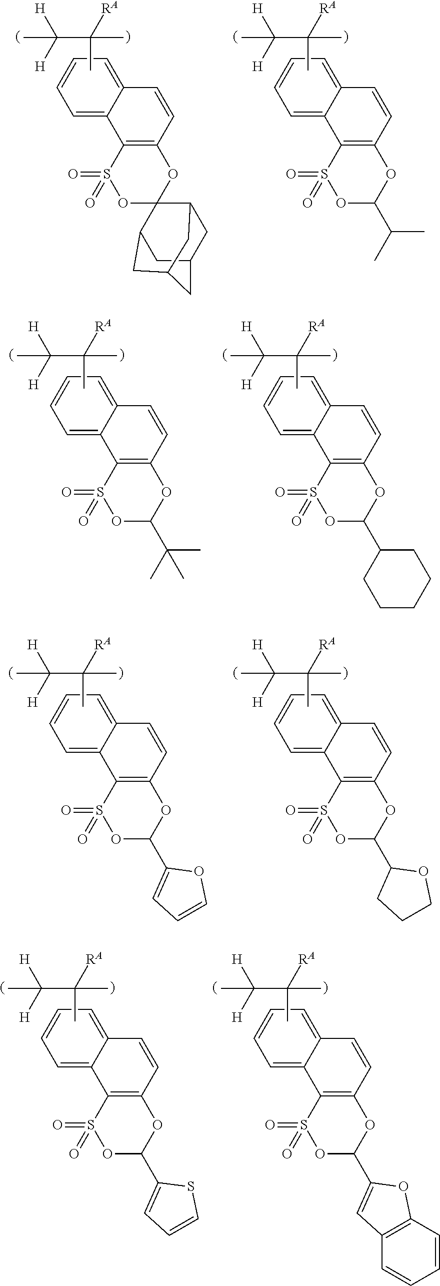

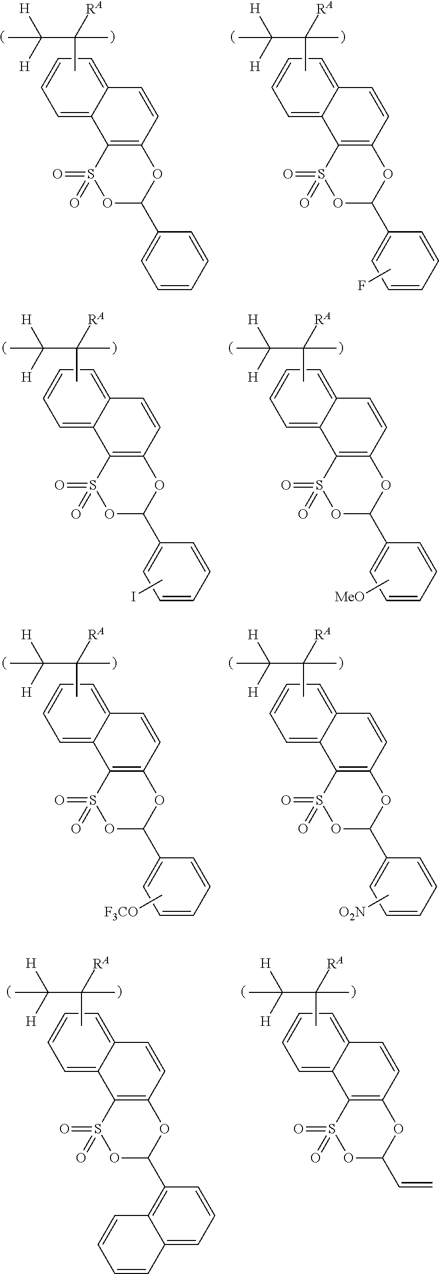

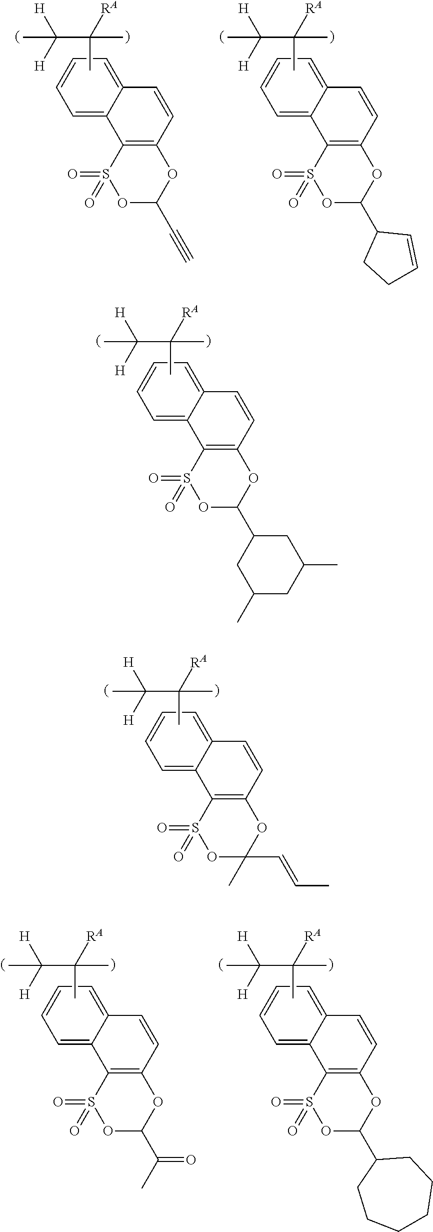

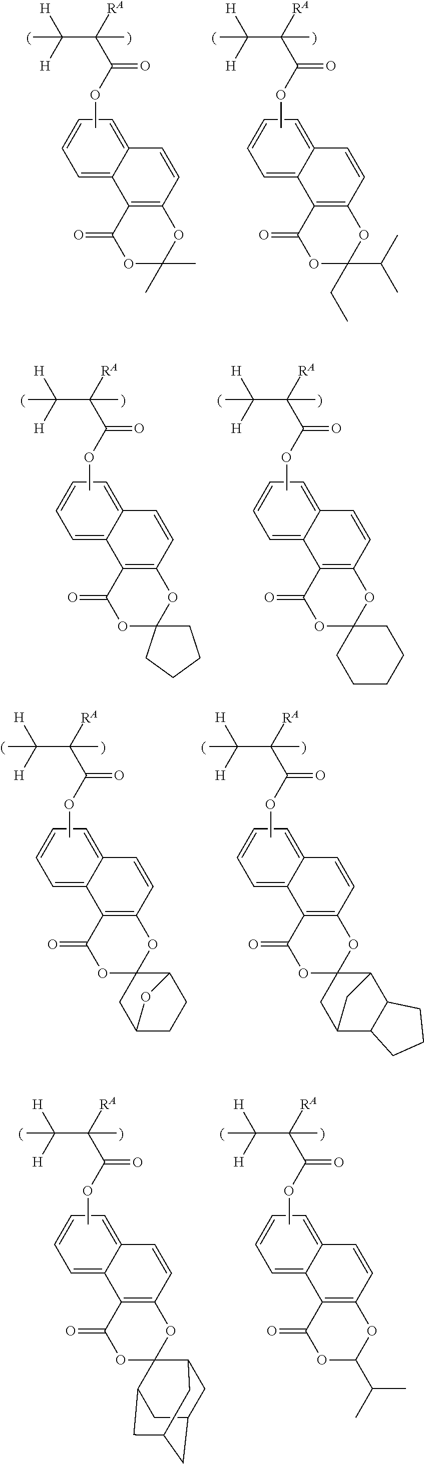

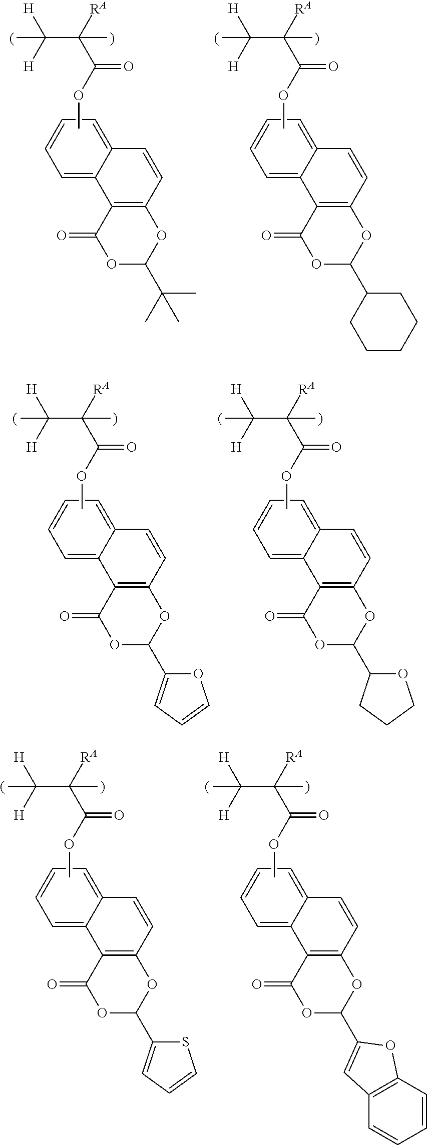

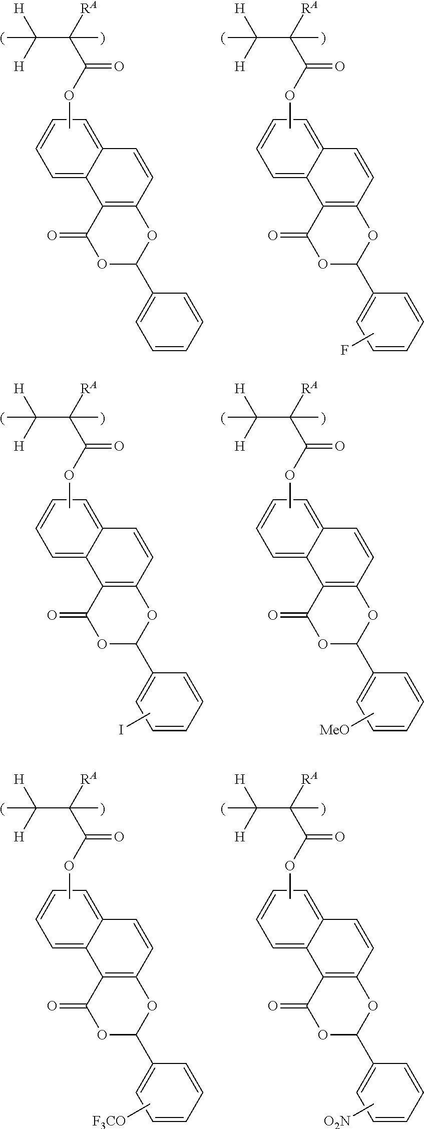

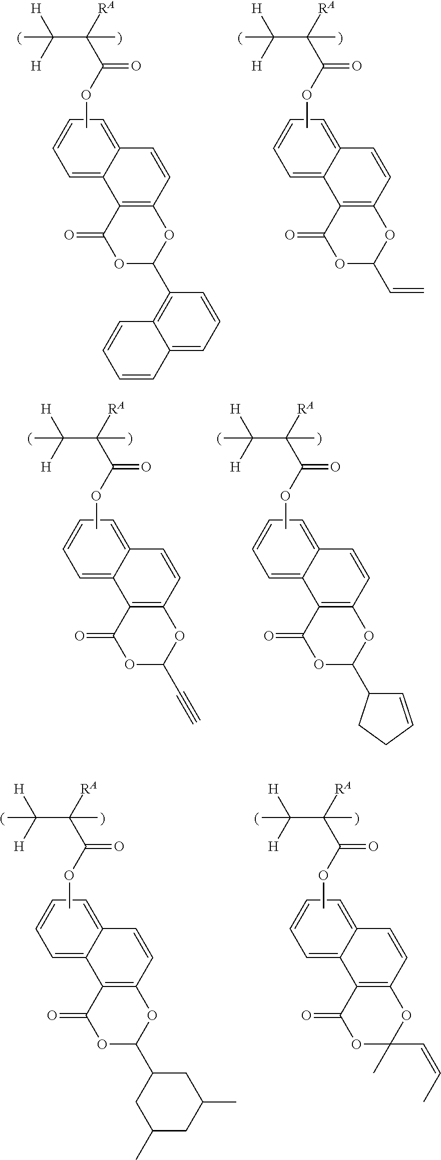

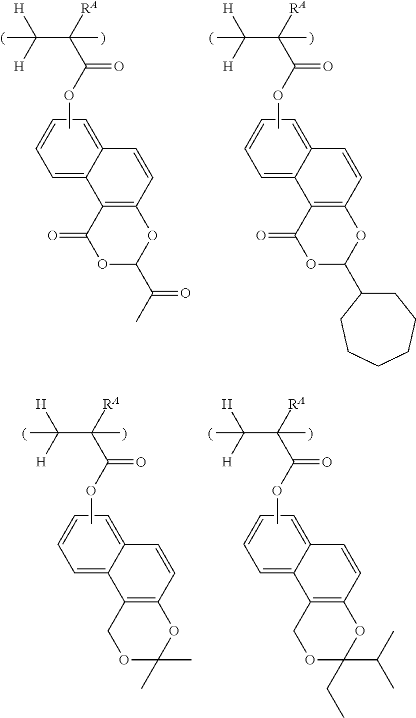

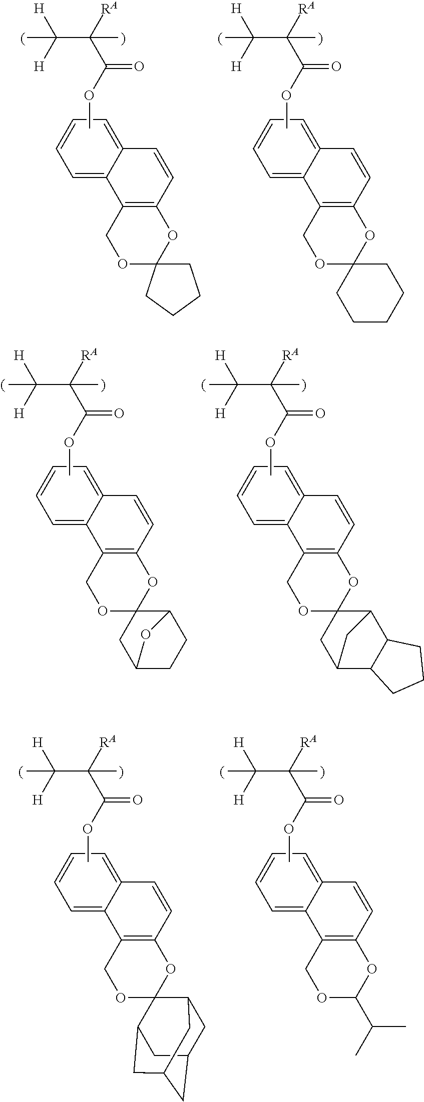

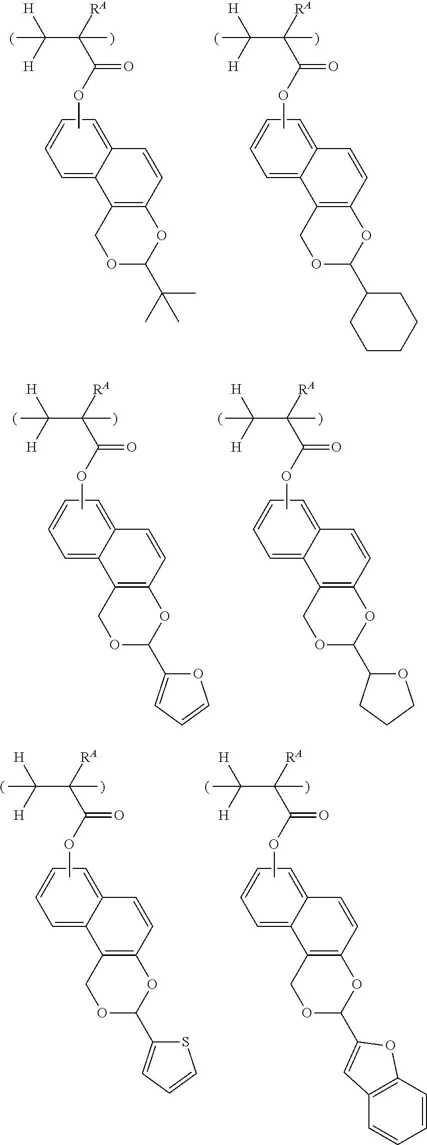

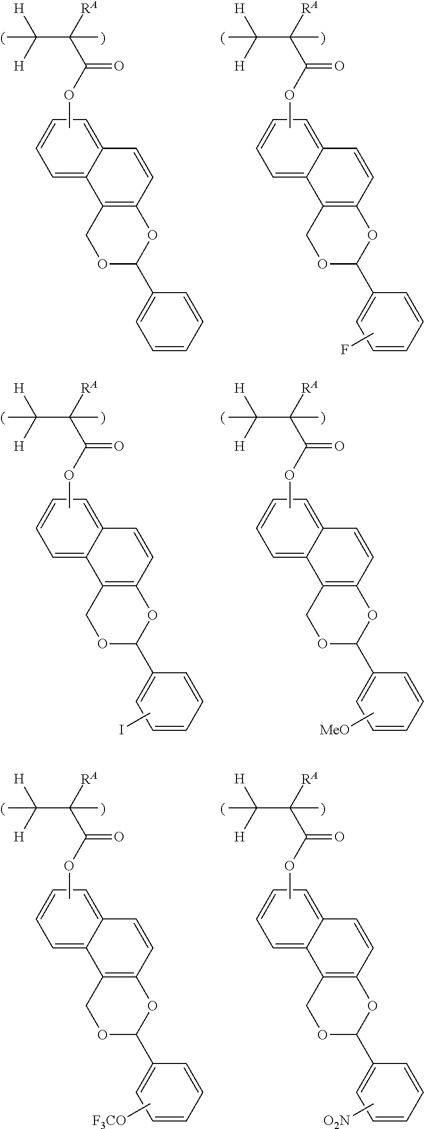

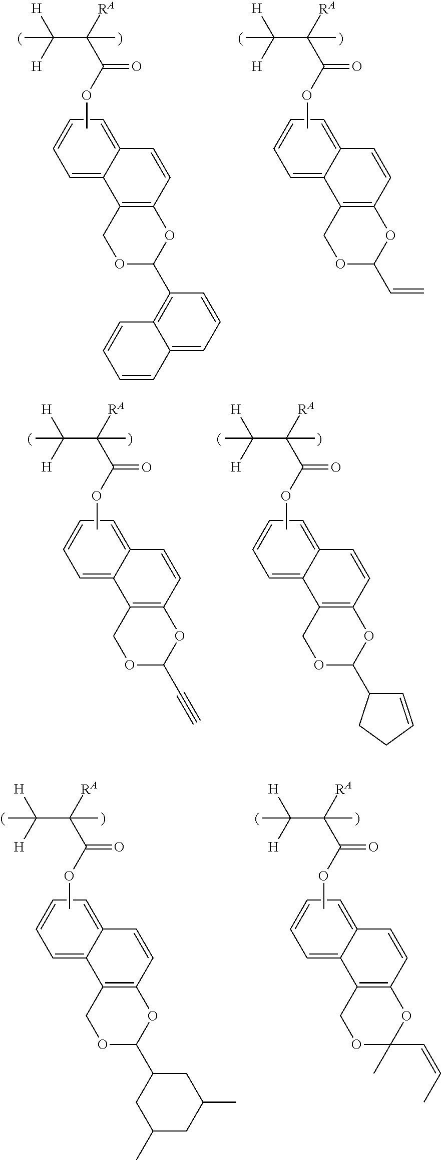

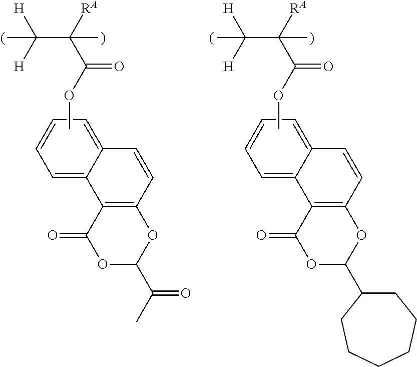

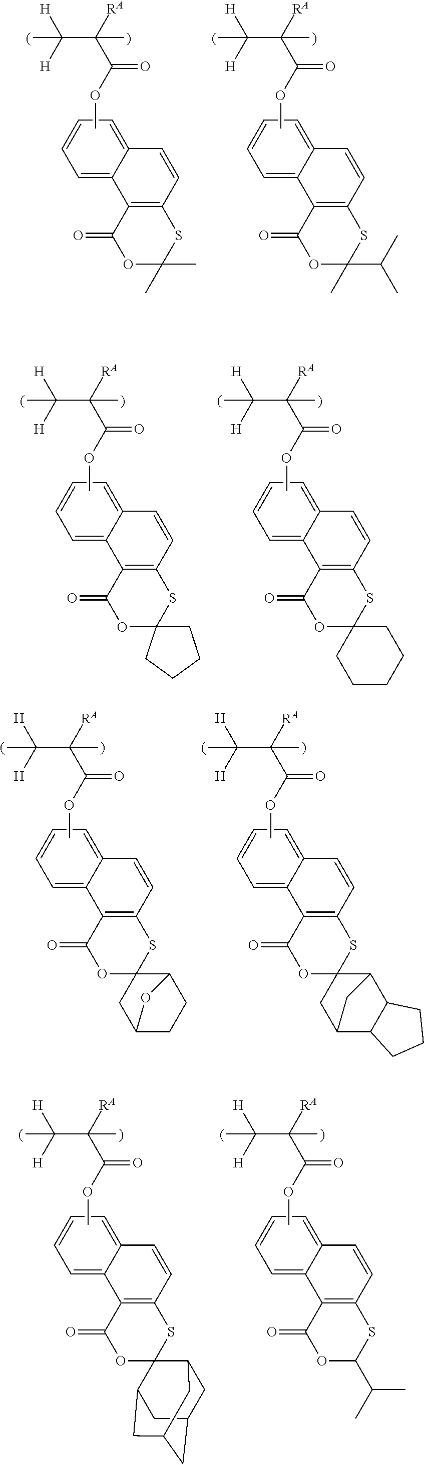

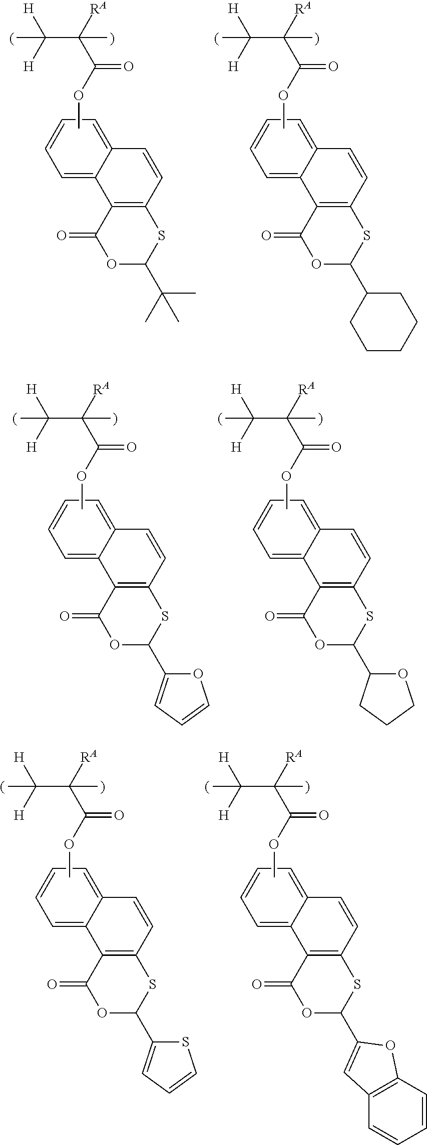

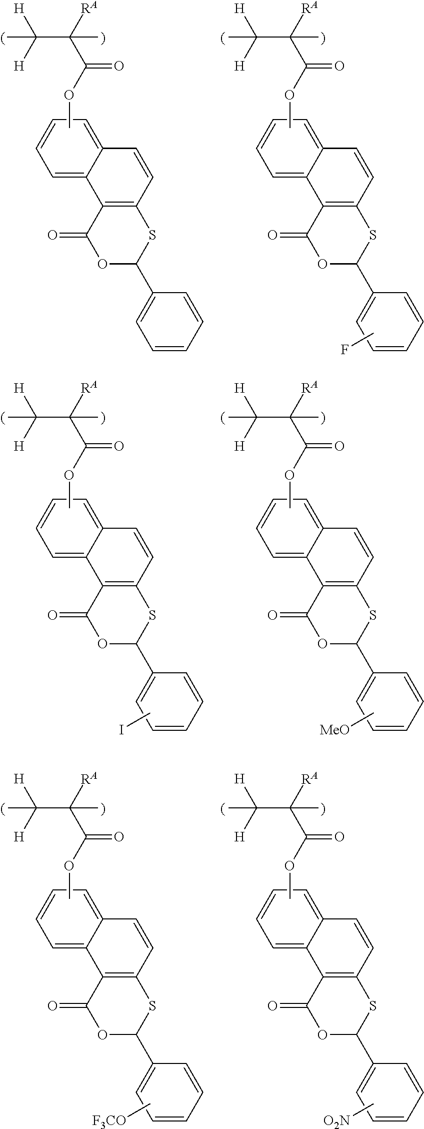

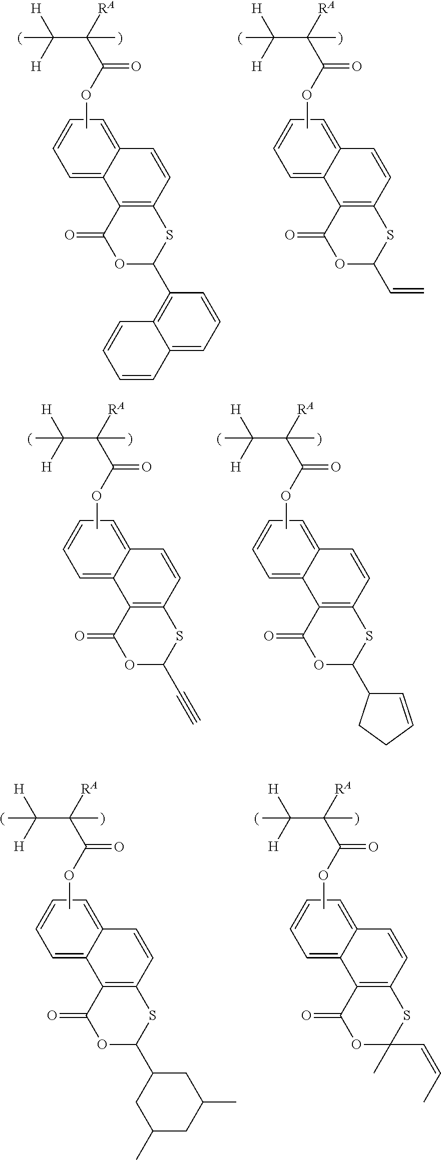

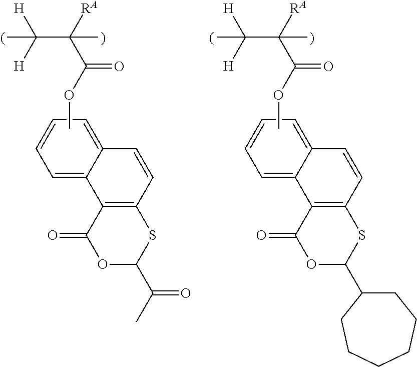

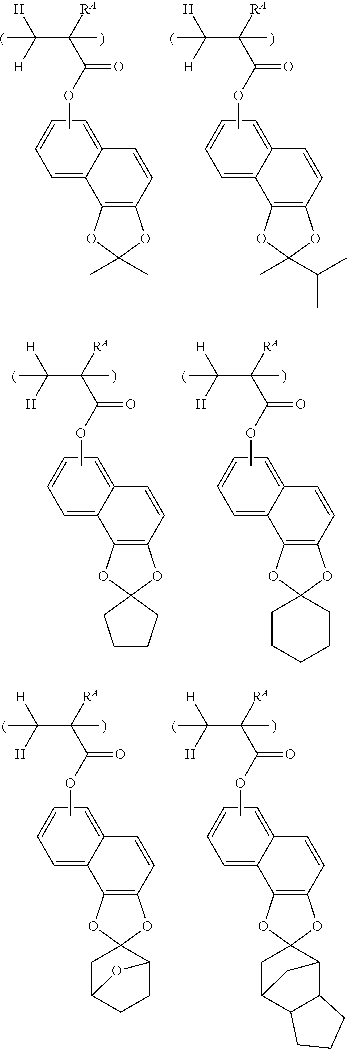

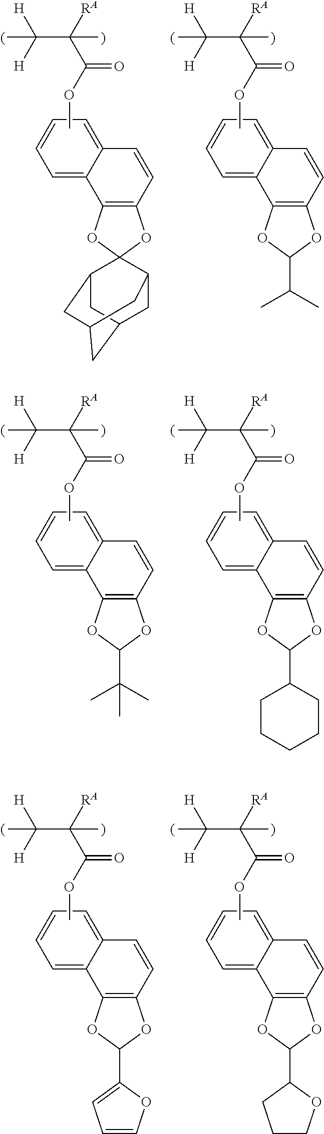

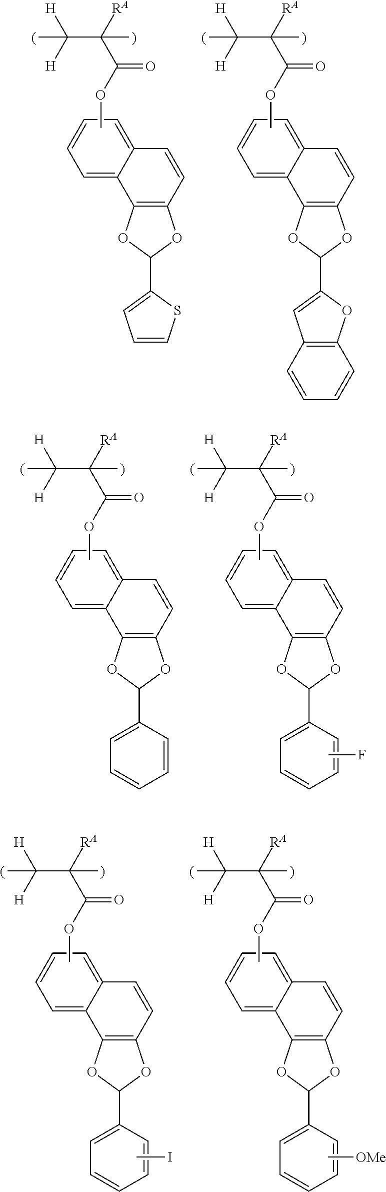

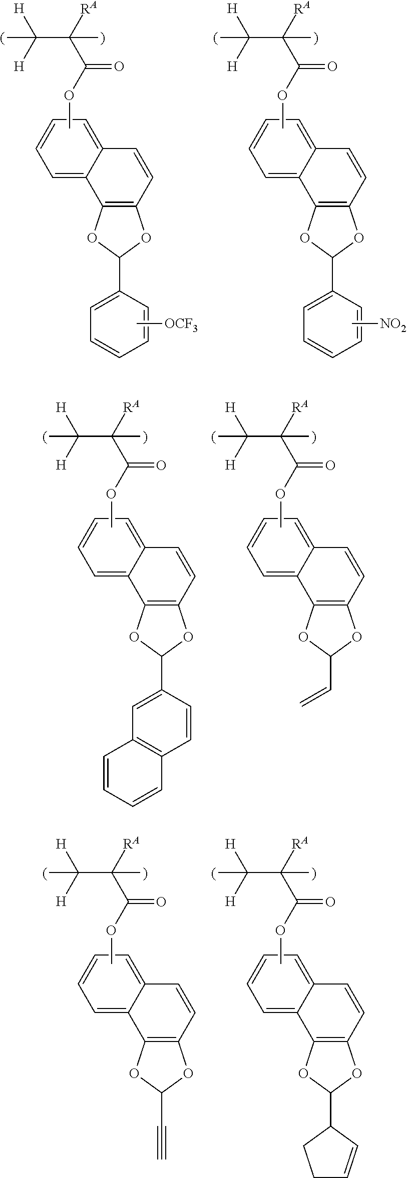

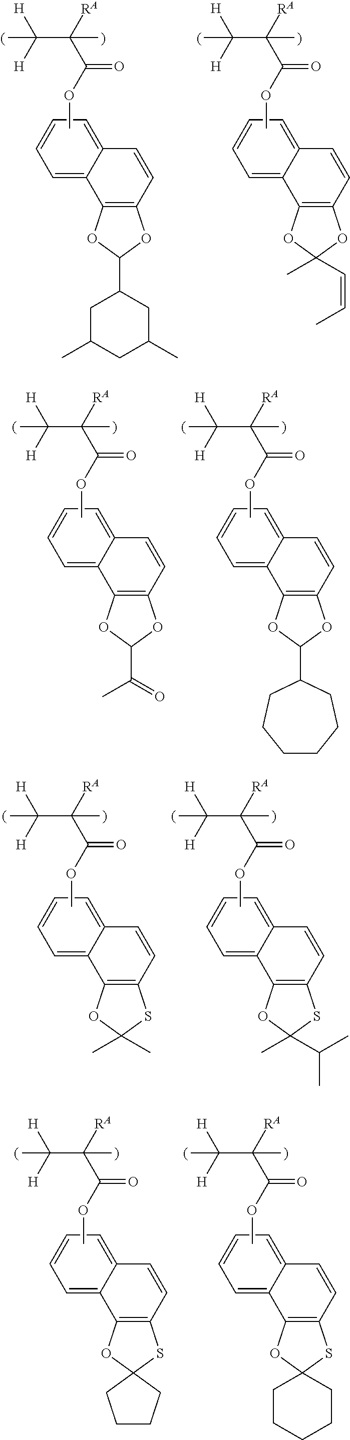

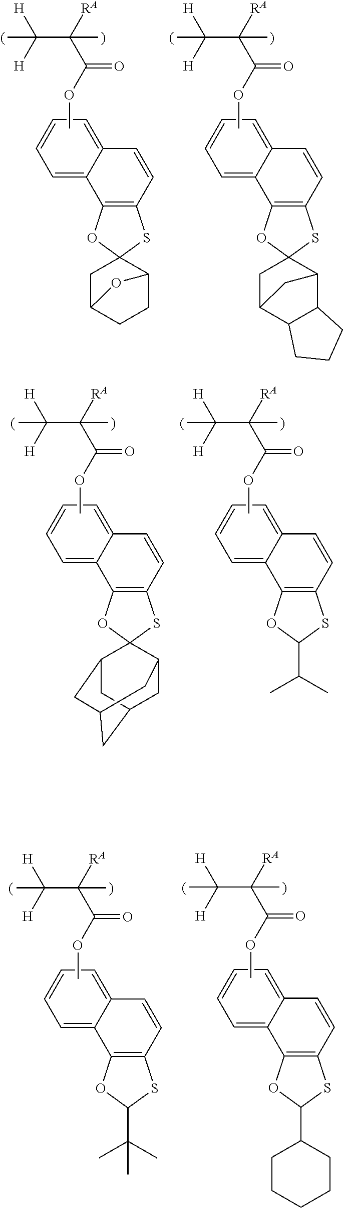

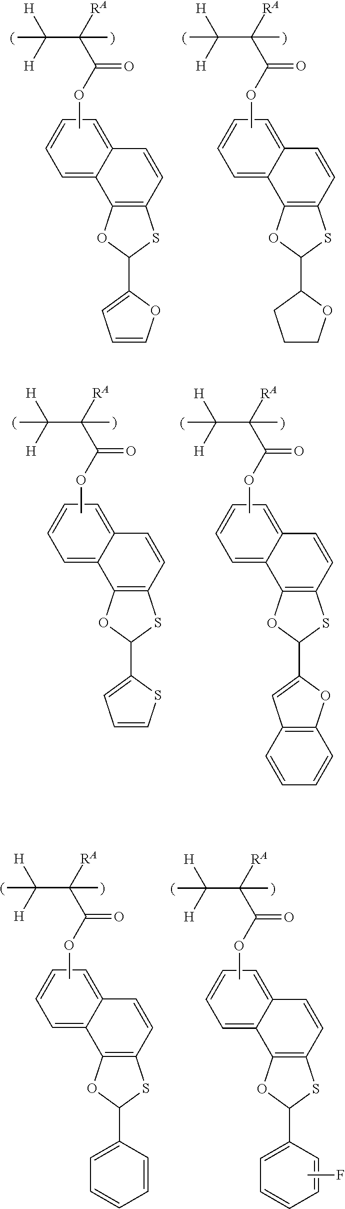

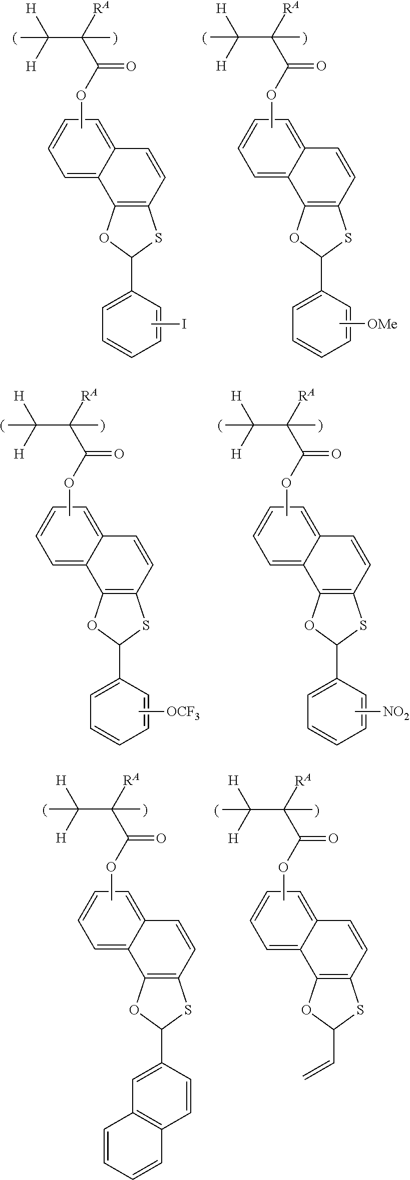

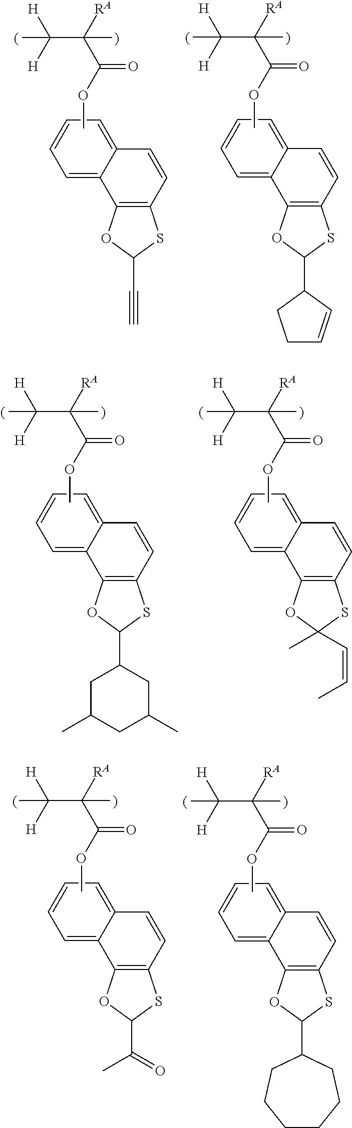

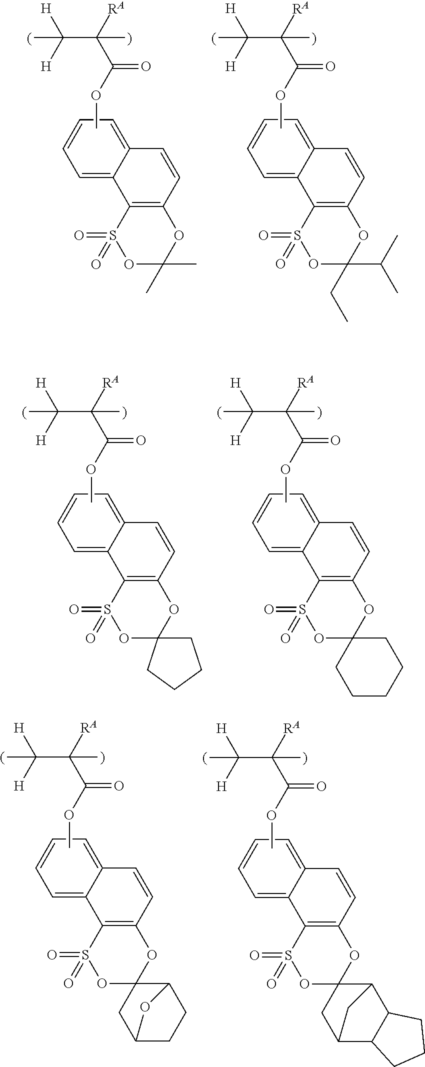

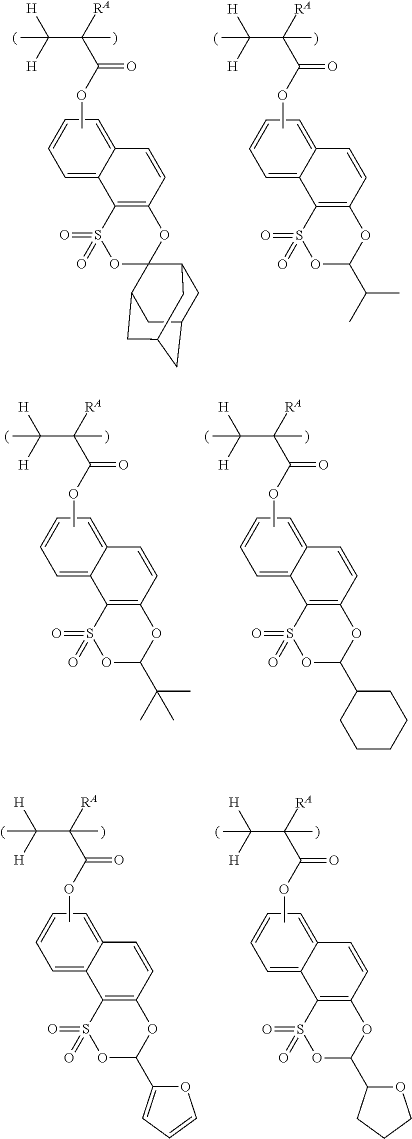

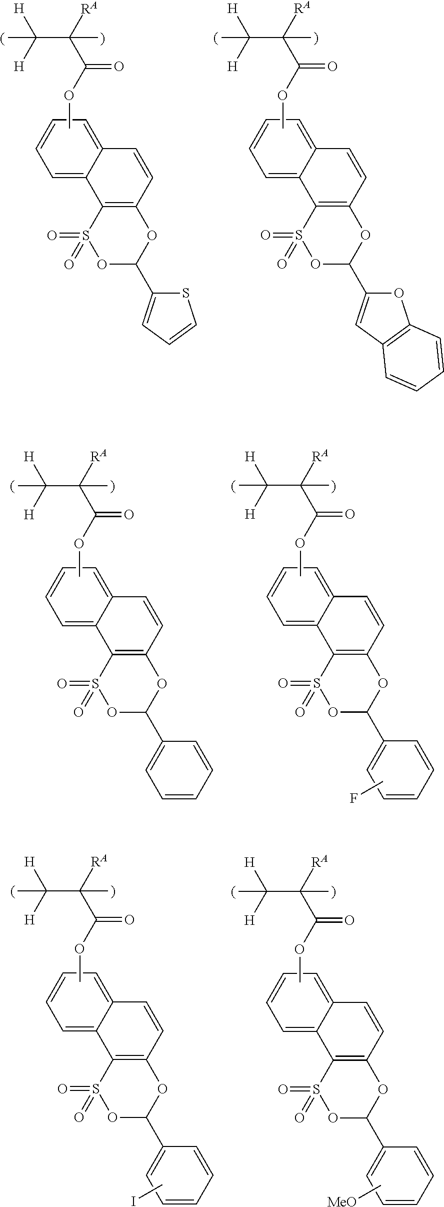

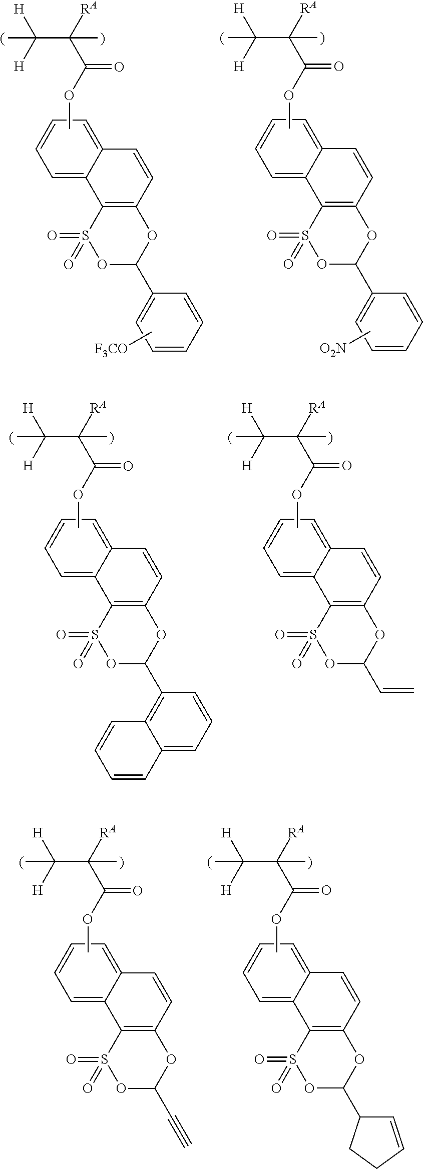

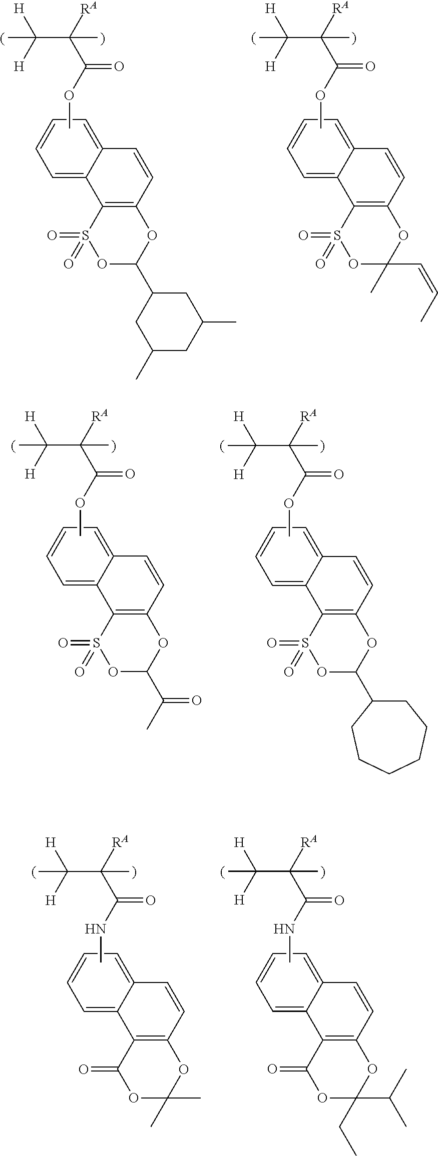

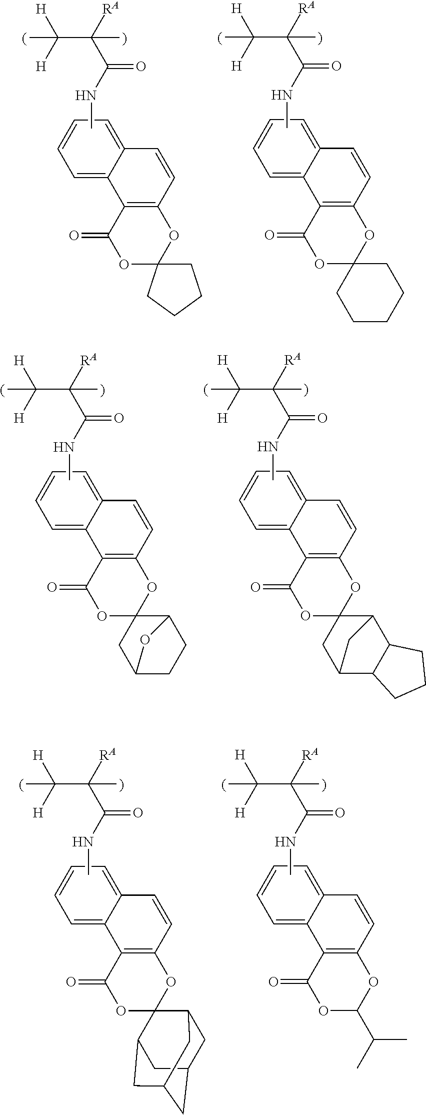

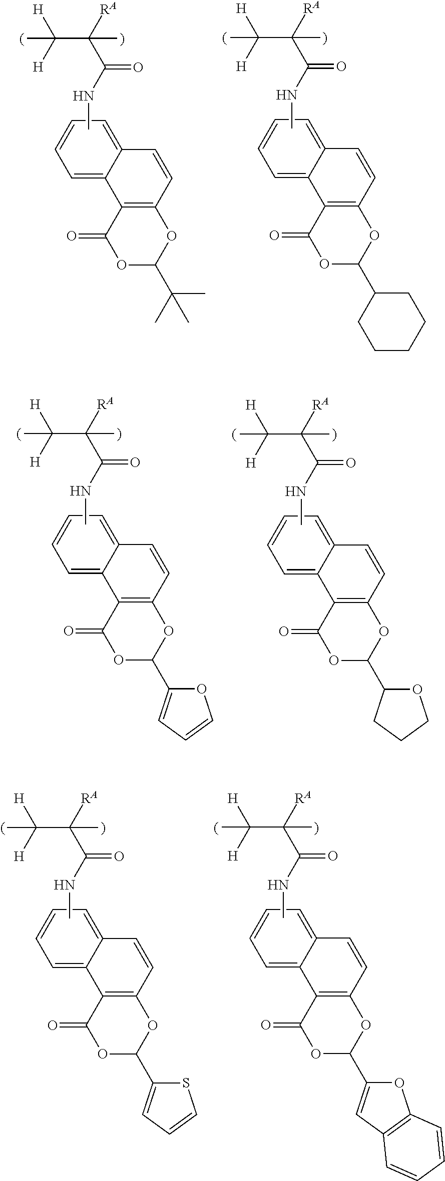

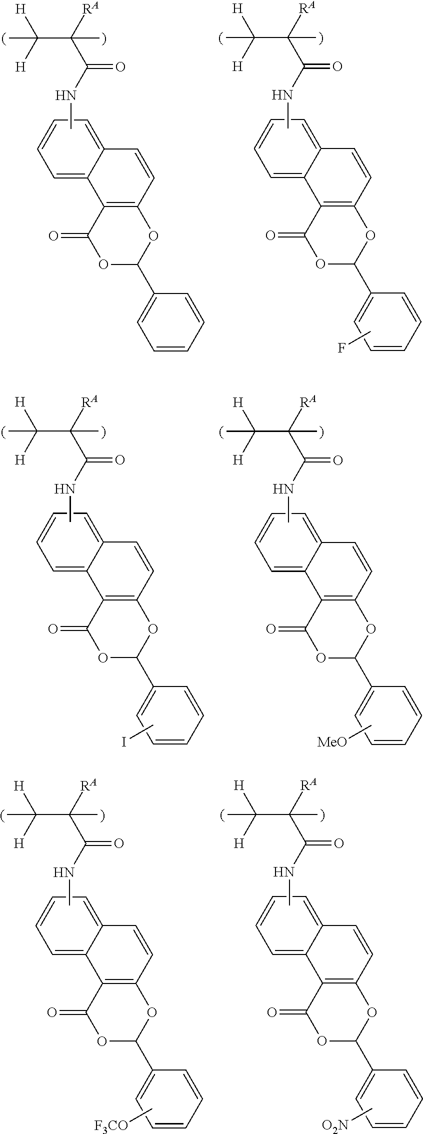

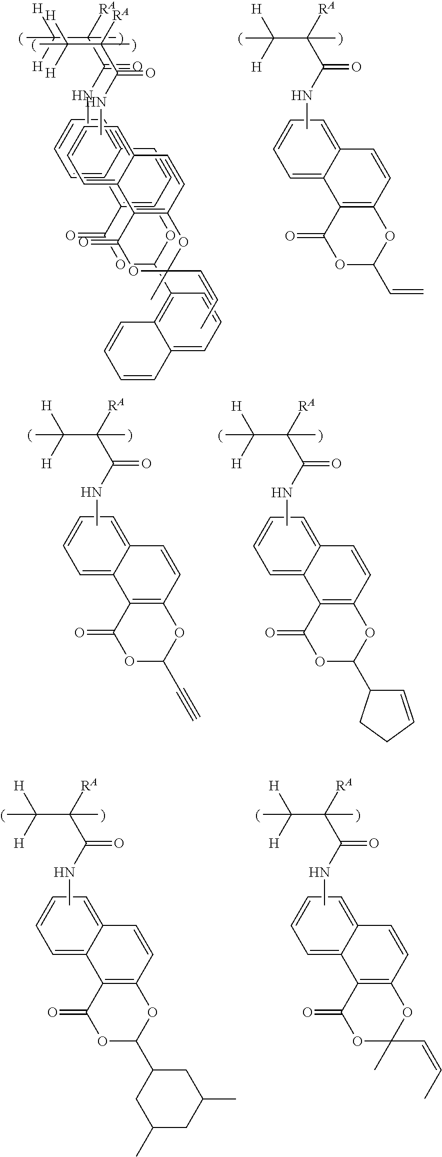

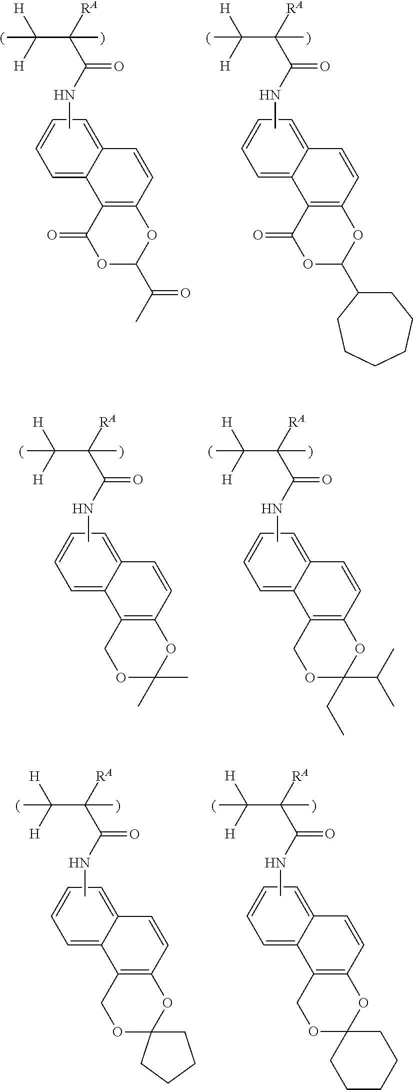

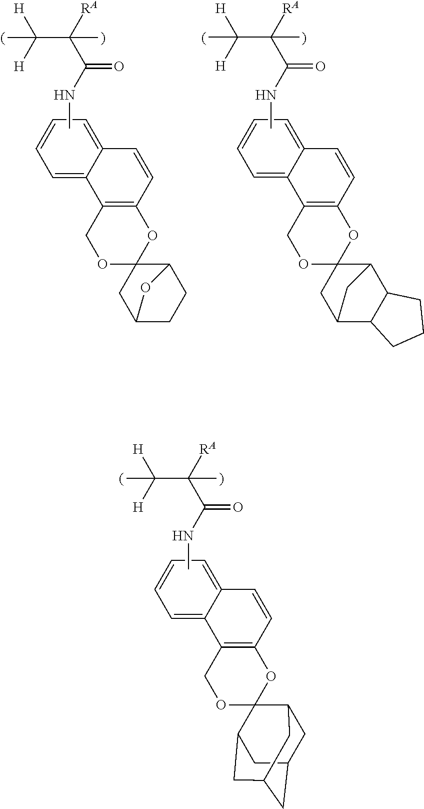

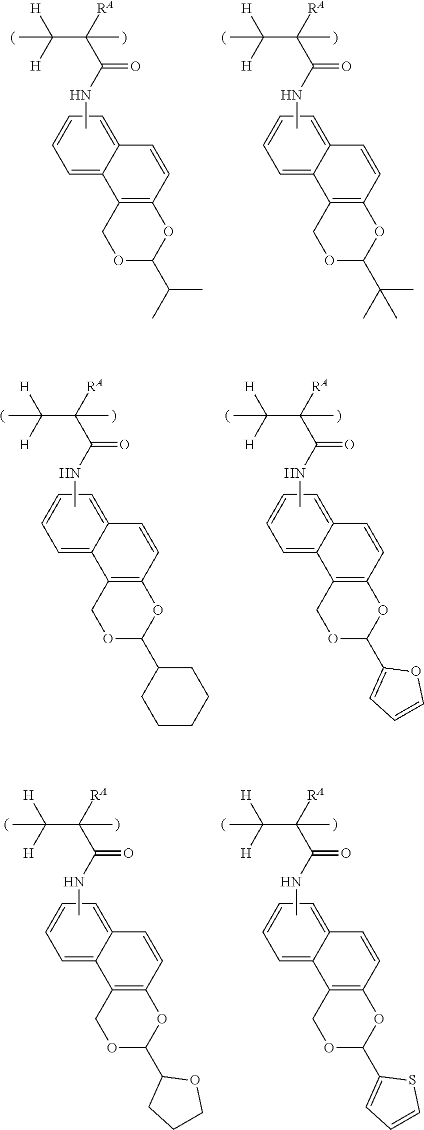

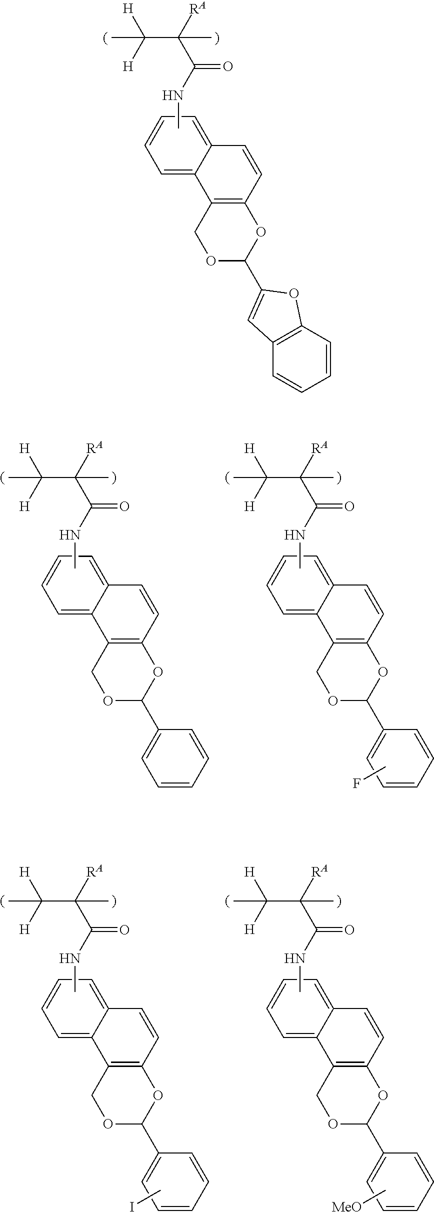

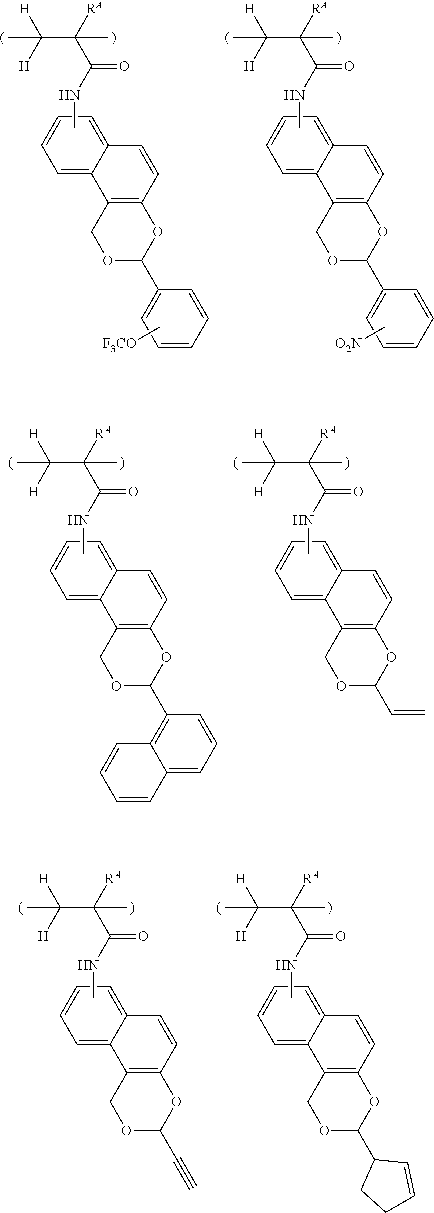

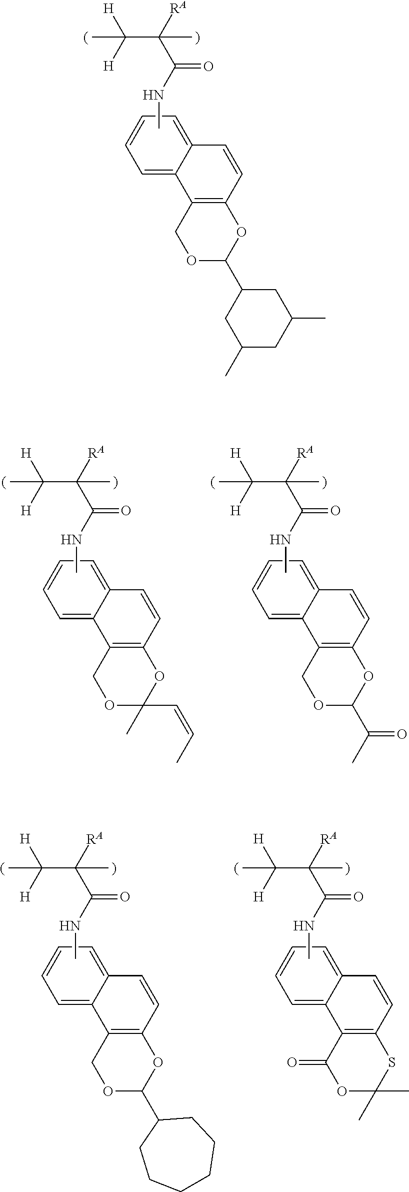

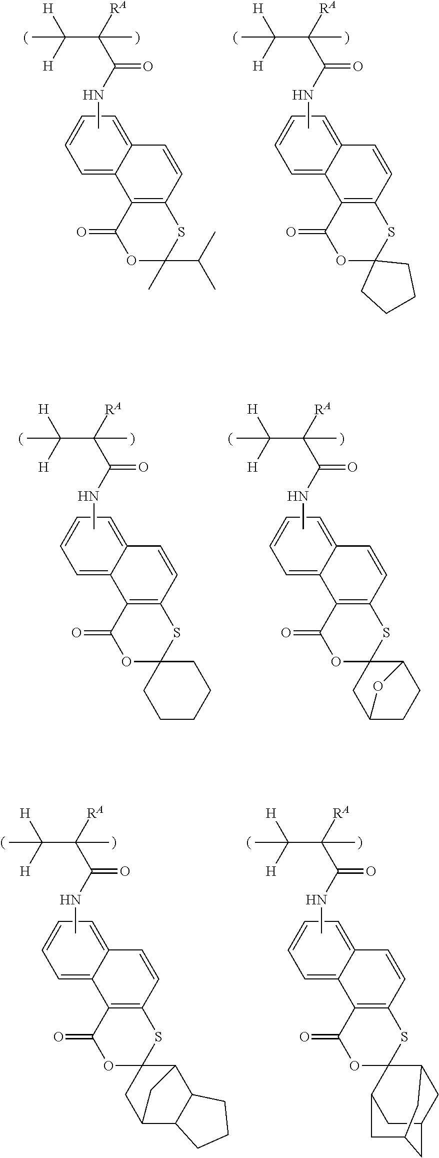

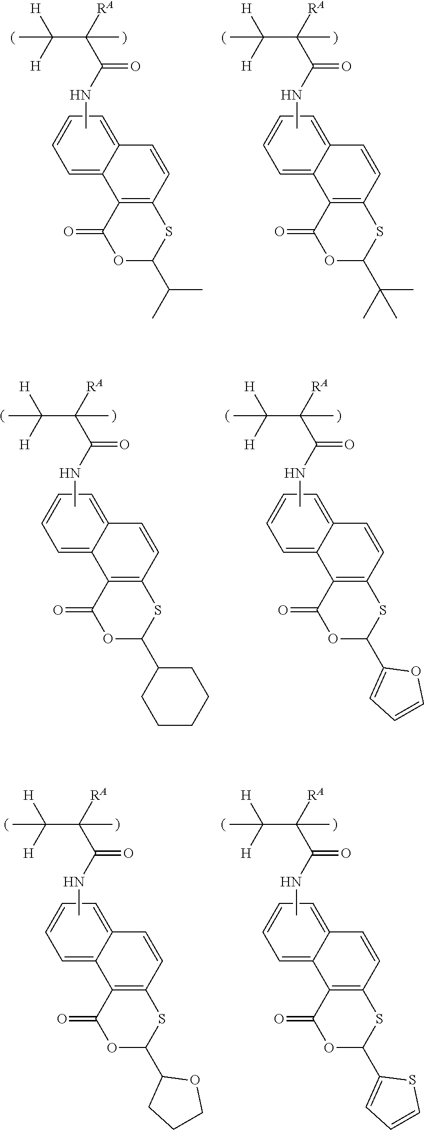

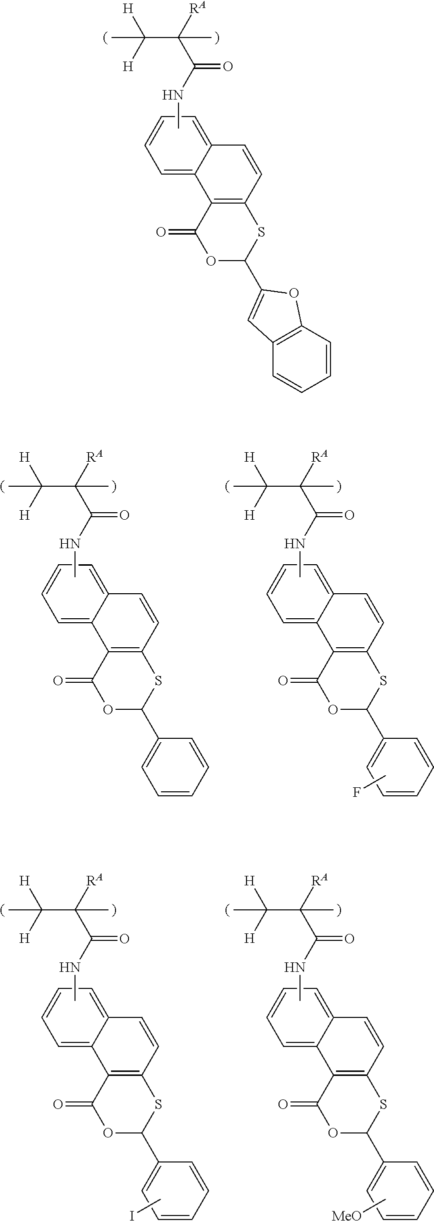

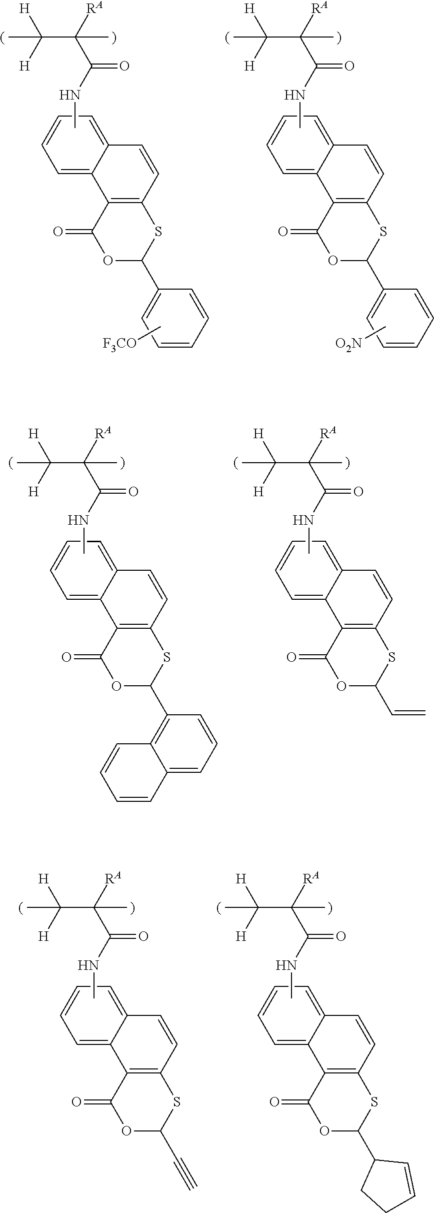

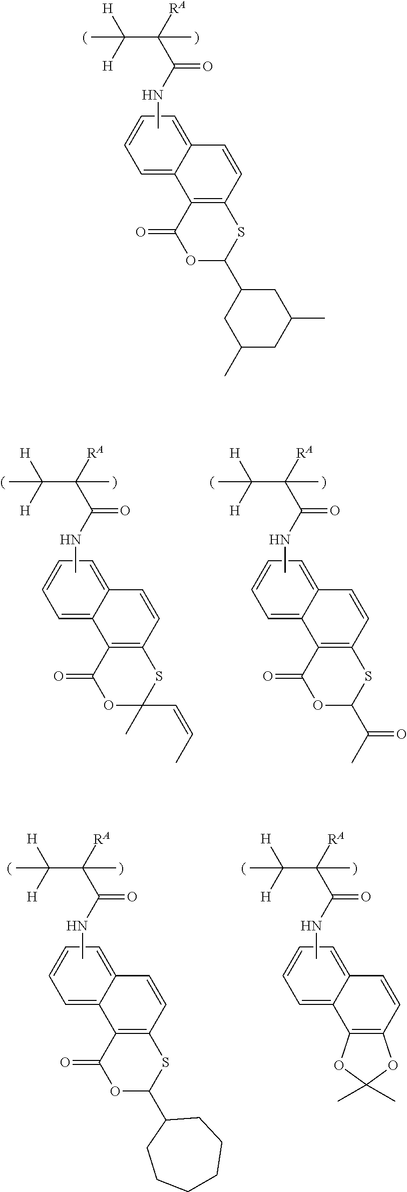

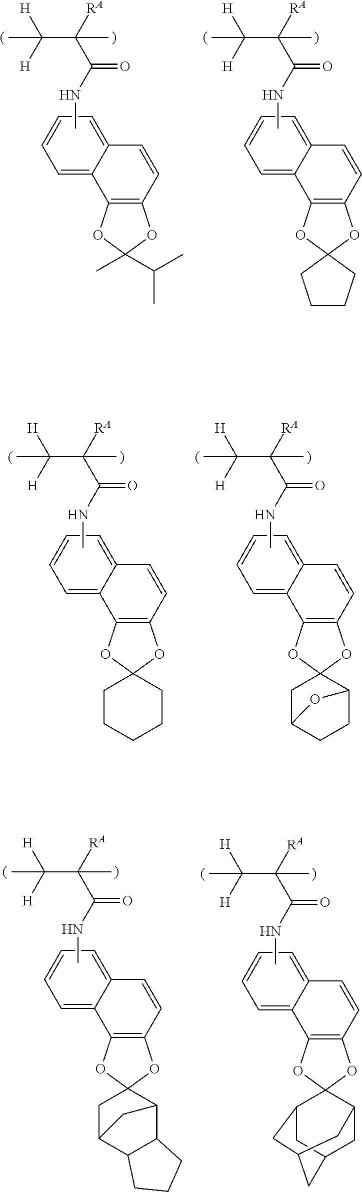

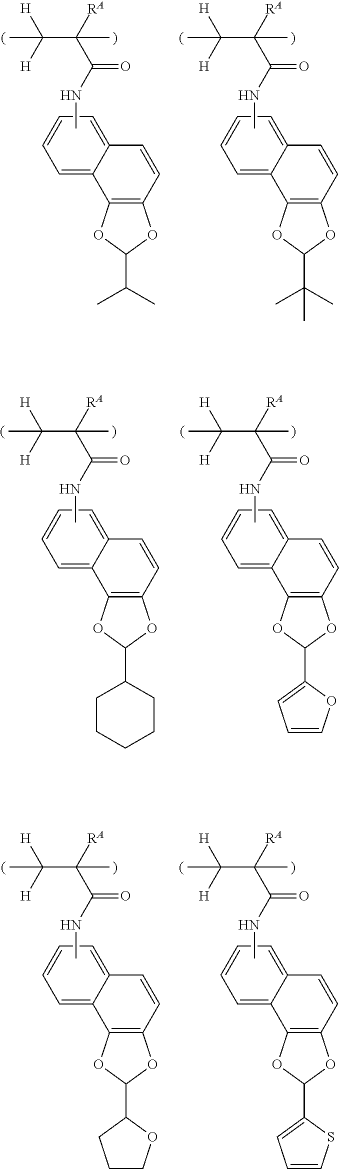

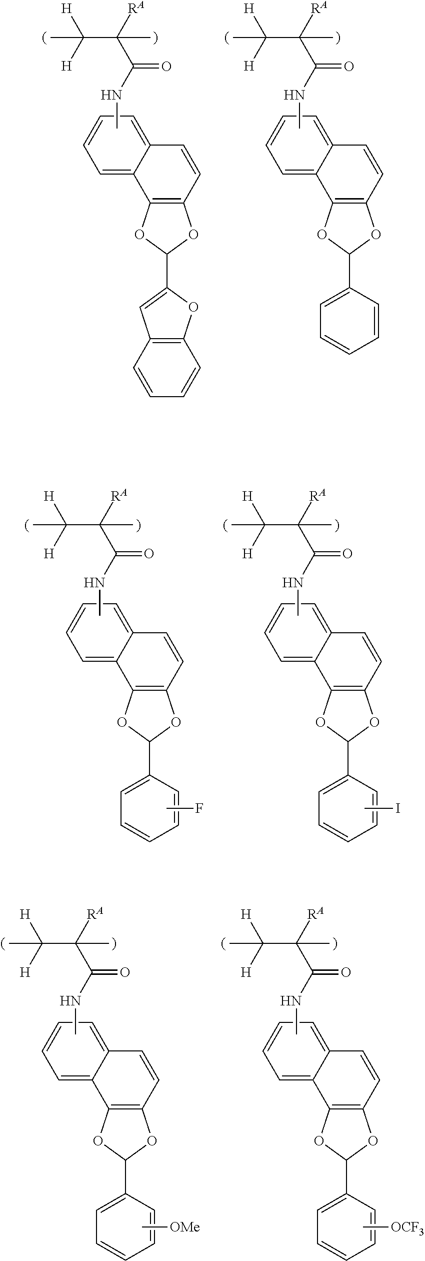

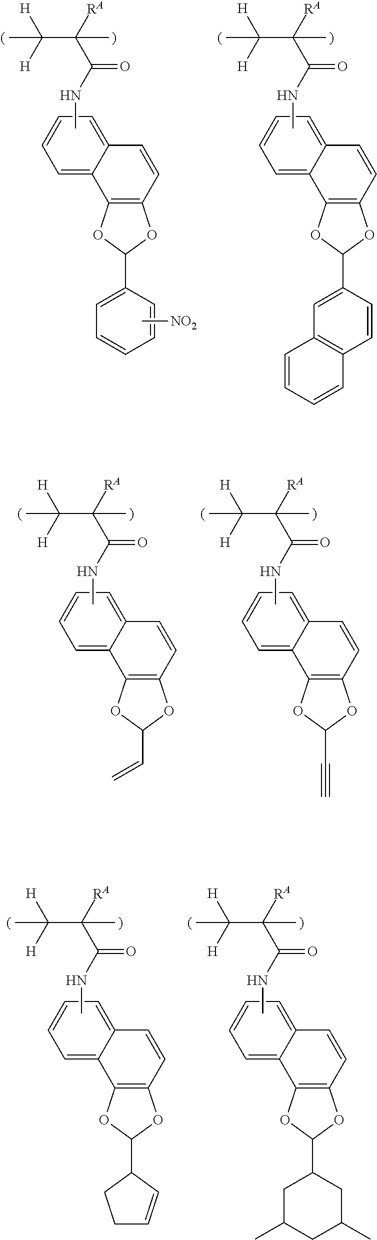

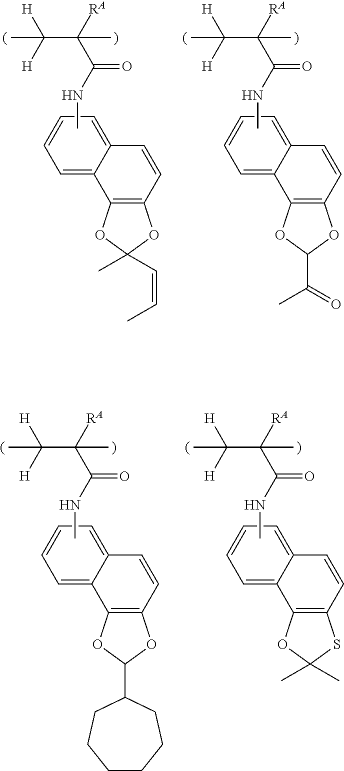

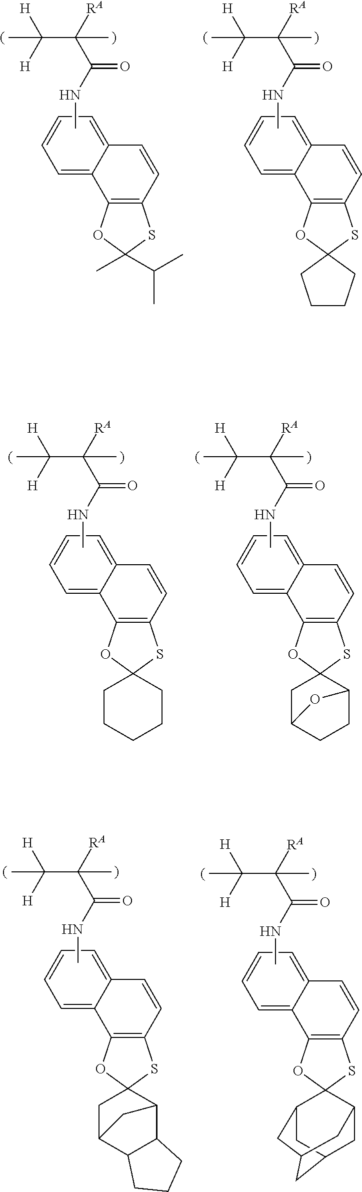

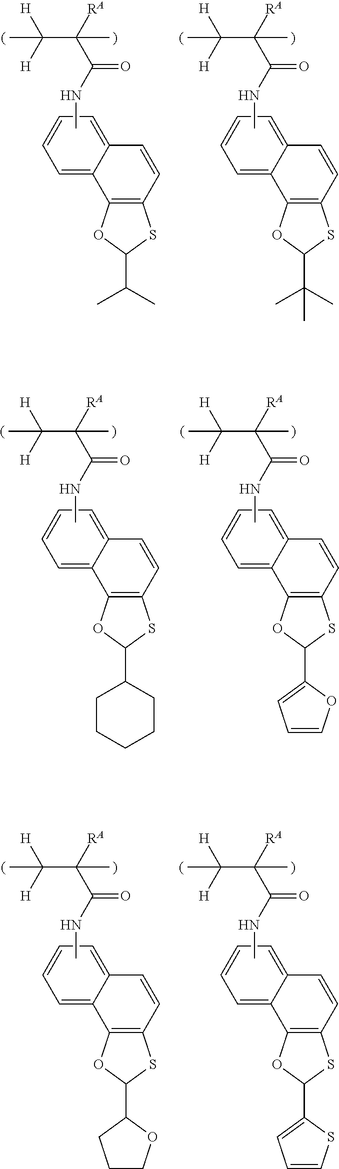

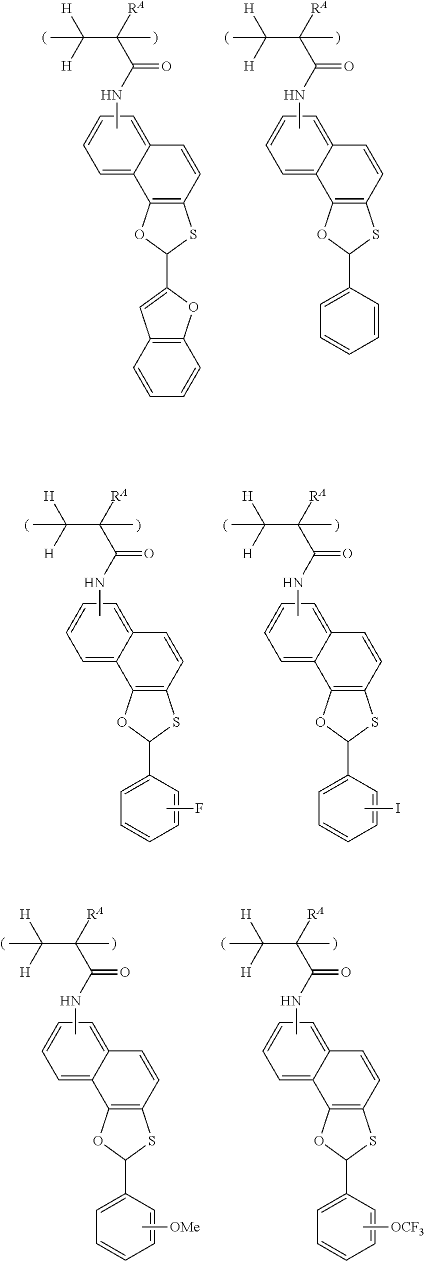

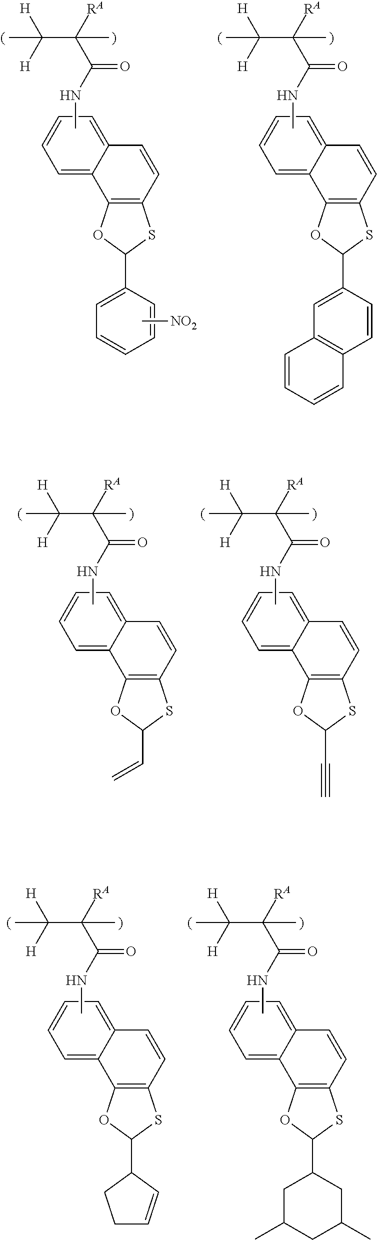

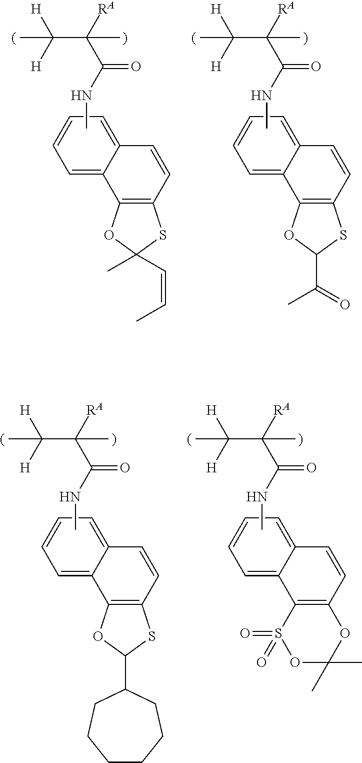

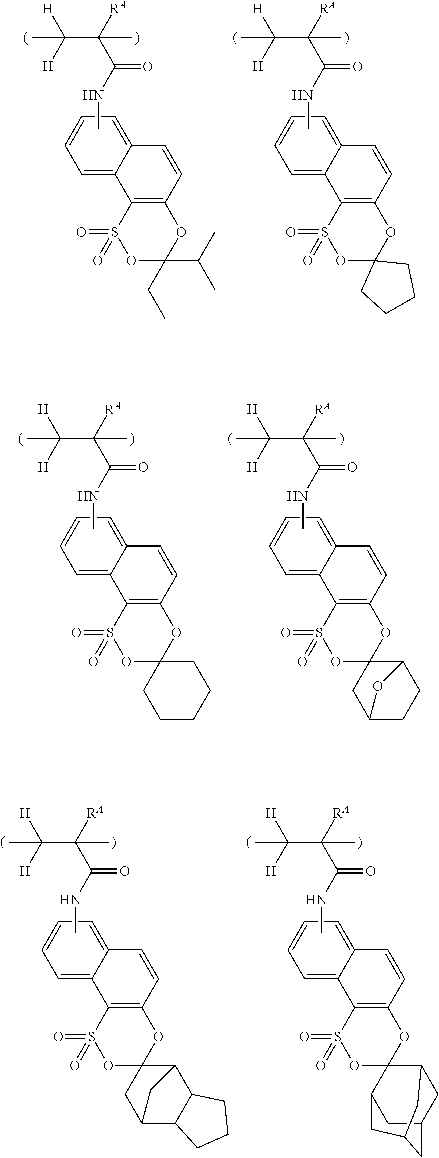

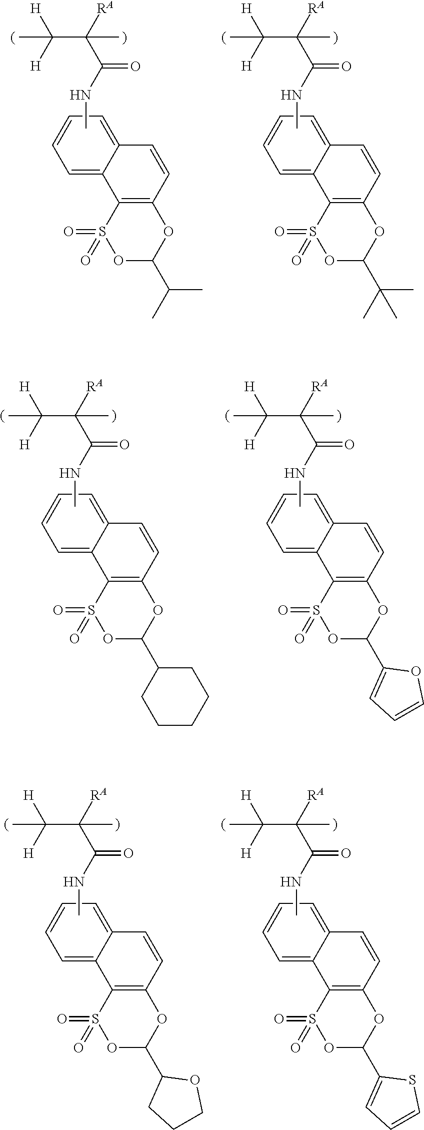

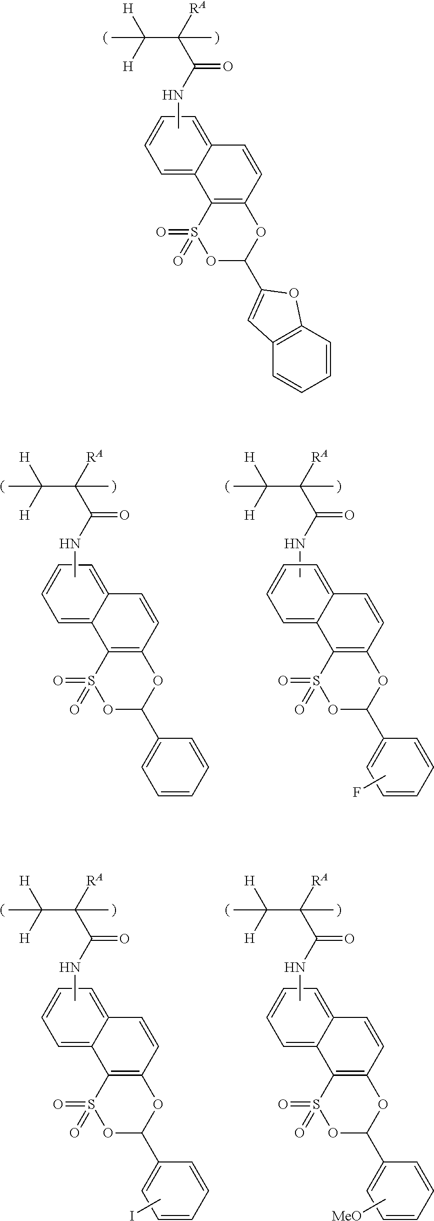

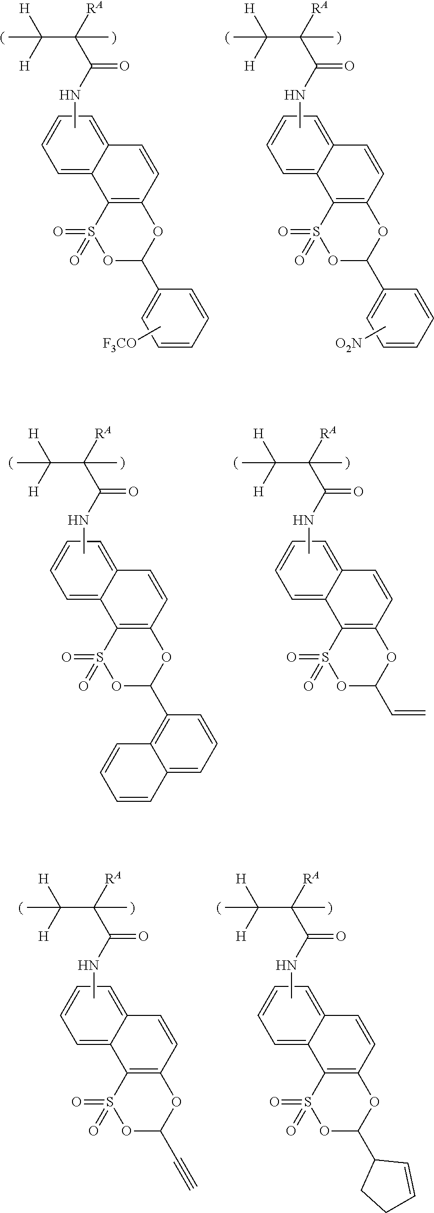

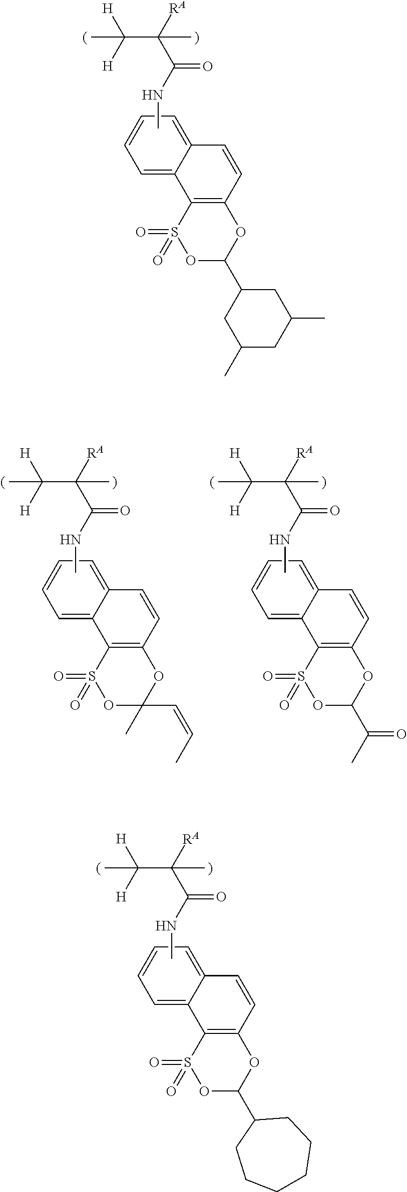

When the chemically amplified resist composition of the invention is of negative tone, the base polymer does not contain an acid labile group. That is, the chemically amplified resist composition does not contain the above-mentioned repeat units (a1) to (a3) or the below-mentioned repeat unit (d).

When the chemically amplified resist composition of the invention is of negative tone, the base polymer preferably contains a repeat unit having the formula (a4) (hereinafter also referred to as repeat unit (a4)).

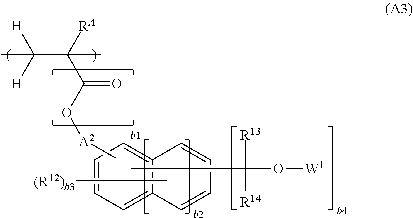

The repeat unit (a4) is a repeat unit in which-O-WA causes an elimination reaction by the action of an acid generated from an acid generator when irradiated with high-energy radiation to induce insolubilization in an alkaline developer and a crosslinking reaction between polymers. By the action of the repeat units (a3) and (a4), the negative resist formation reaction can be more efficiently carried out, so that resolution performance can be improved.

In the formula (a4), b11 is 0 or 1, b12 is 0, 1, or 2, b13 is an integer meeting 0≤b3≤5+2 (b2)-b4, and b14 is 1, 2, or 3.

In the formula (a4), R^{A}'s are each independently a hydrogen atom, a fluorine atom, a methyl group, or a trifluoromethyl group.

In the formula (a4), R¹⁵ is a halogen atom, or a C₁-C₂₀ hydrocarbyl group which may contain a heteroatom, Specific examples of the halogen atom of R³¹ include a fluorine atom, a chlorine atom, a bromine atom, and an iodine atom. The C₁-C₂₀ hydrocarbyl group of R¹⁵ may be saturated or unsaturated and straight, branched, or cyclic. Specific examples thereof are as exemplified above for the hydrocarbyl group of R¹ in the description of the formula (1). When b13 is 2 or more, a plurality of R¹⁵'s may be identical or different.

In the formula (a4), R¹⁶ and R¹⁷ are each independently a hydrogen atom, a C₁-C₁₅ saturated hydrocarbyl group, or a C₆-Cis aryl group, the hydrocarbyl group may be substituted with a hydroxy group or a C₁-C₆ saturated hydrocarbyloxy group, and the aryl group may have a substituent. It is excluded that R¹⁶ and R¹⁷ are a hydrogen atom at the same time. R¹⁶ and R¹⁷ may bond together to form a ring with the carbon atom to which they are attached, some —CH₂— in the ring may be replaced by —O— or —S—. Preferred examples of R¹⁶ and R¹⁷ include alkyl groups such as a methyl group, an ethyl group, a propyl group, a butyl group, and structural isomers thereof, and those in which some hydrogen atoms thereof are substituted with a hydroxy group or a saturated hydrocarbyloxy group.

In the formula (a4), A1 is a single bond or a C₁-C₁₀ saturated hydrocarbylene group, and some —CH₂— in the saturated hydrocarbylene group may be replaced by —O—. The saturated hydrocarbylene group may be straight, branched, or cyclic, and specific examples thereof include a methylene group, alkanediyl groups such as an ethane-1,2-diyl group, a propane-1,3-diyl group, a butane-1,4-diyl group, a pentane-1,5-diyl group, a hexane-1,6-diyl group, and structural isomers thereof; cyclic saturated hydrocarbylene groups such as a cyclopropanediyl group, a cyclobutanediyl group, a cyclopentanediyl group, and a cyclohexanediyl group; and a group obtained by combining these groups. In the case where the saturated hydrocarbylene group contains an ether bond, when b11 in the formula (a4) is 1, the ether bond may be inserted at any position except between a carbon atom at α-position and a carbon atom at β-position with respect to an ester oxygen atom. When b11 is 0, an atom attached to the backbone is an ether oxygen atom, and a second ether bond may be inserted at any position except between a carbon atom at α-position and a carbon atom at β-position with respect to the ether oxygen atom.

In the formula (a4), WA's are each independently a hydrogen atom, a C₁-C₁₀ aliphatic hydrocarbyl group, a C₂-C₁₀ aliphatic hydrocarbylcarbonyl group, or a C₆-C₁₅ aryl group, and the aryl group may have a substituent.

The C₁-C₁₀ aliphatic hydrocarbyl group of WA may be saturated or unsaturated and straight, branched, or cyclic. Specific examples thereof include C₁-C₁₀ alkyl groups such as a methyl group, an ethyl group, a n-propyl group, an isopropyl group, a n-butyl group, an isobutyl group, a sec-butyl group, a tert-butyl group, a n-pentyl group, a neopentyl group, n-hexyl group, a n-heptyl group, a n-octyl group, a 2-ethylhexyl group, a n-nonyl group, and a n-decyl group; C₃-C₁₀ cyclic saturated hydrocarbyl groups such as a cyclopentyl group and a cyclohexyl group; C₂-C₁₀ alkenyl groups such as a vinyl group, a 1-propenyl group, a 2-propenyl group, a butenyl group, and a hexenyl group; and C₃-C₁₀ cyclic unsaturated hydrocarbyl groups such as a cyclohexenyl group. The hydrocarbyl moiety of the C₂-C₁₀ aliphatic hydrocarbylcarbonyl group of WA may be saturated or unsaturated and straight, branched, or cyclic. Specific examples thereof include C₁-C₉ aliphatic hydrocarbyl groups among the specific examples of the C₁-C₁₀ aliphatic hydrocarbyl group described above. Specific examples of the C₆-Cis aryl group of WA include a phenyl group, a naphthyl group, and an anthryl group, and among these groups, a phenyl group is preferred. The aryl group may have a substituent, and specific examples of the substituent include a halogen atom, a C₁-C₆ saturated hydrocarbyl group which may be substituted with a halogen atom, and a C₁-C₆ saturated hydrocarbyloxy group which may be substituted with a halogen atom. The repeat unit (a4) is preferably a group having the formula (a4-1) or (a4-2).

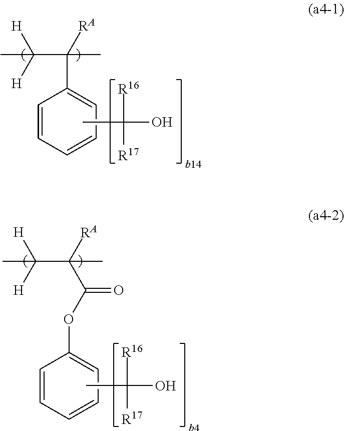

In the formulae, b14, R^{A}, R¹⁶, and R¹⁷ are as defined above.

Specific examples of the repeat unit (a4) are shown below, but not limited thereto. In the following formulae. R^{A} is as defined above. Me is a methyl group, and Ac is an acetyl group.

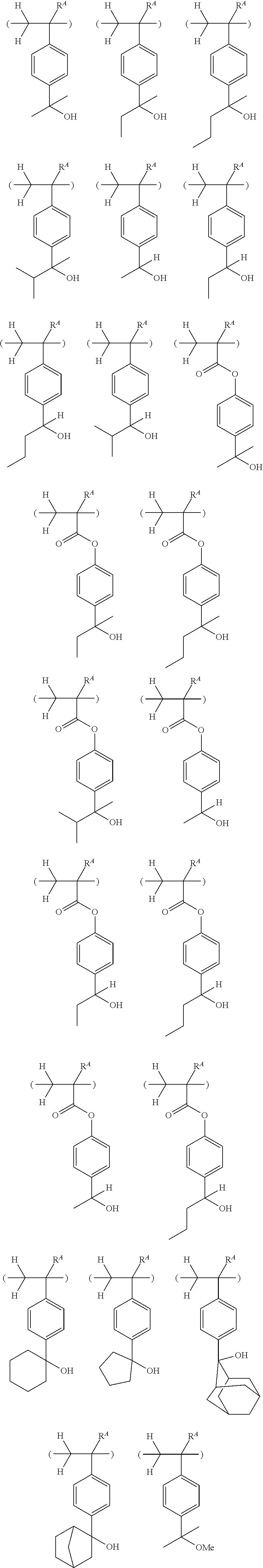

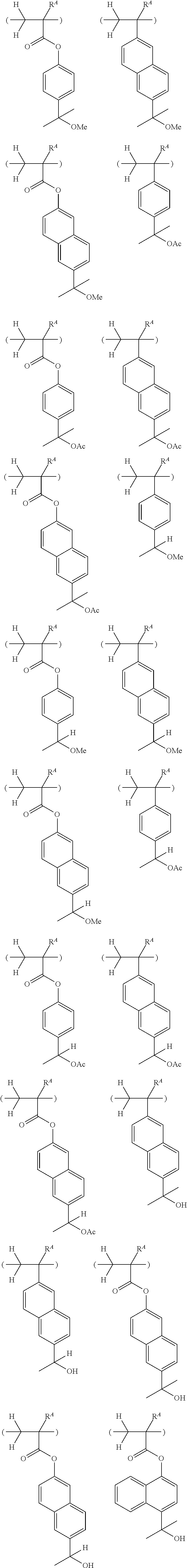

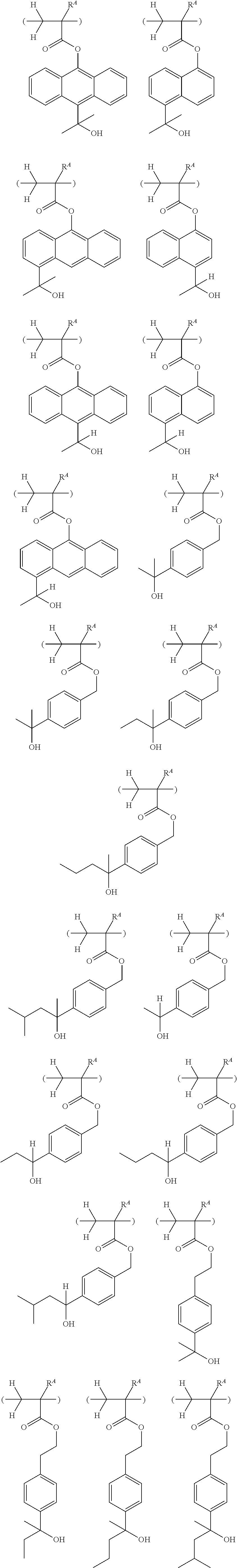

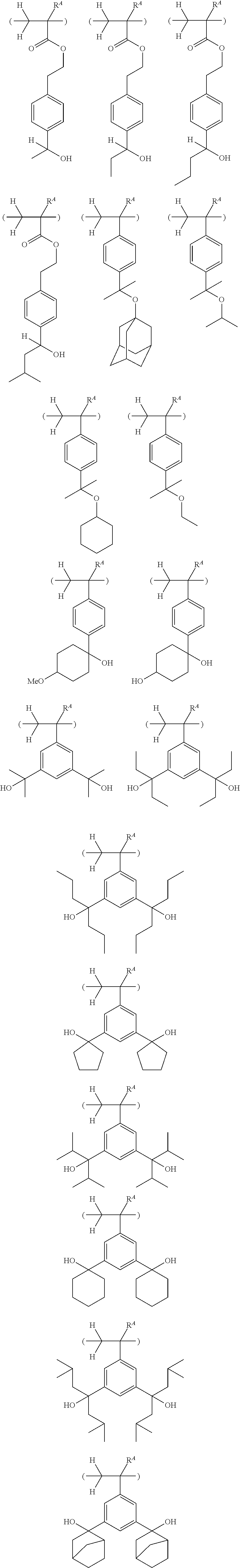

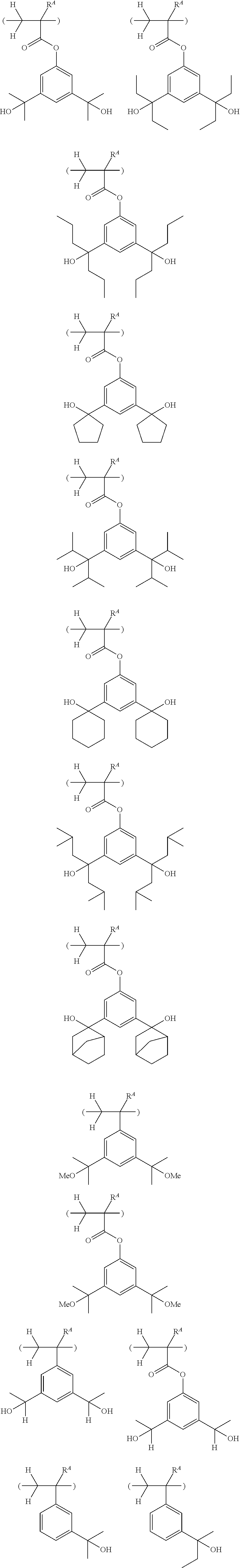

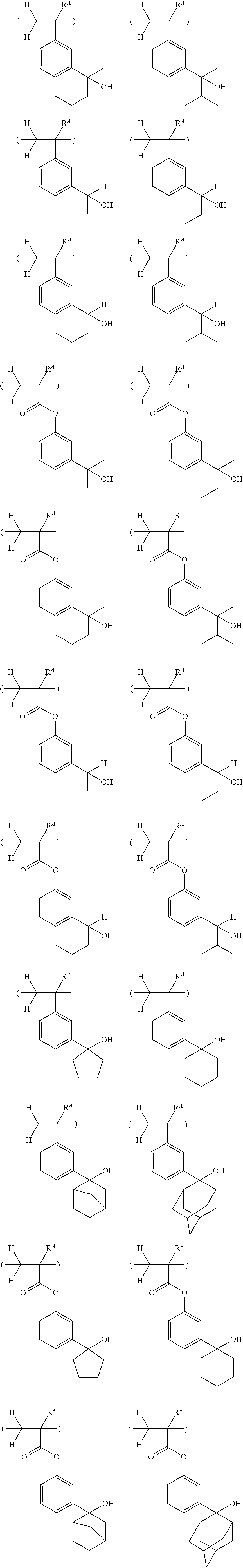

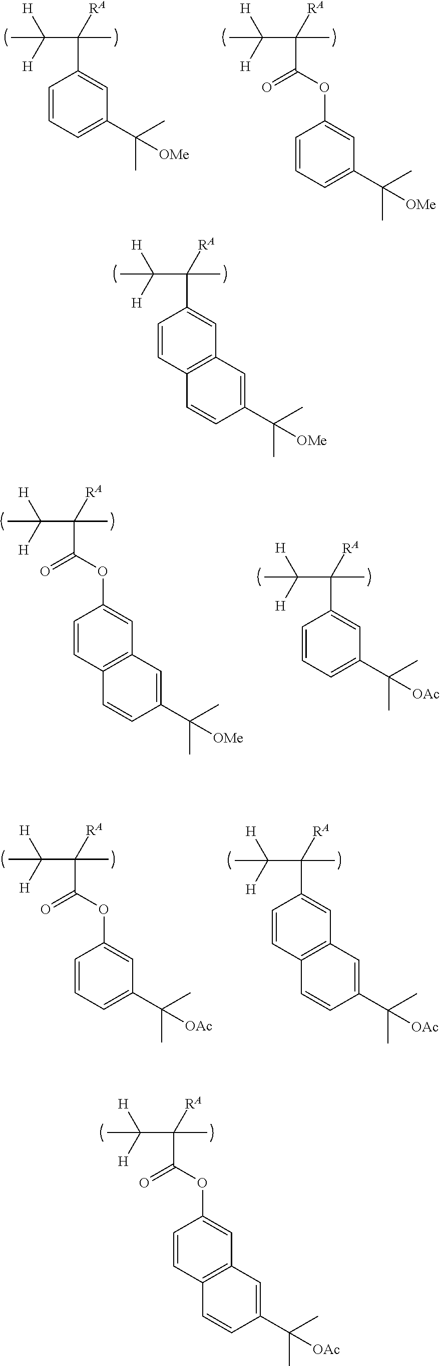

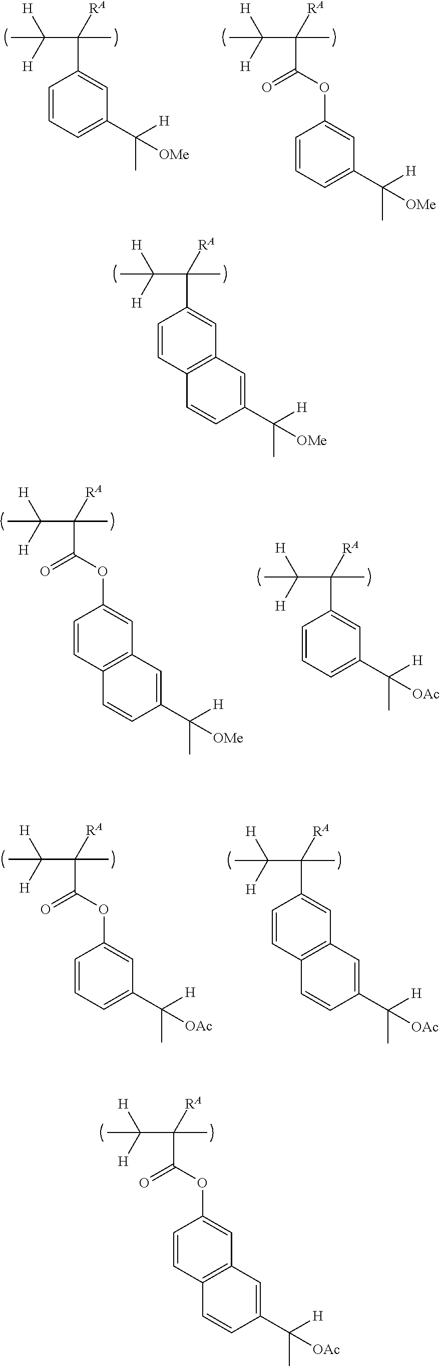

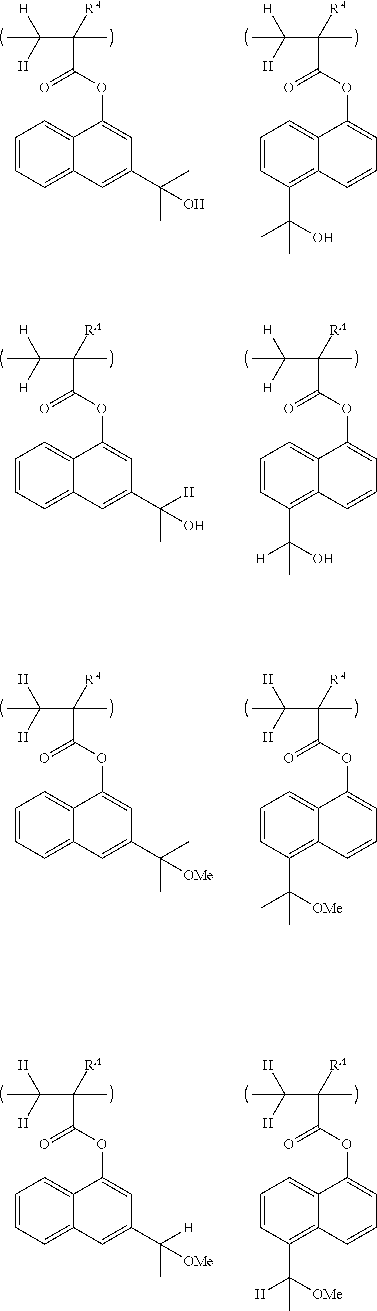

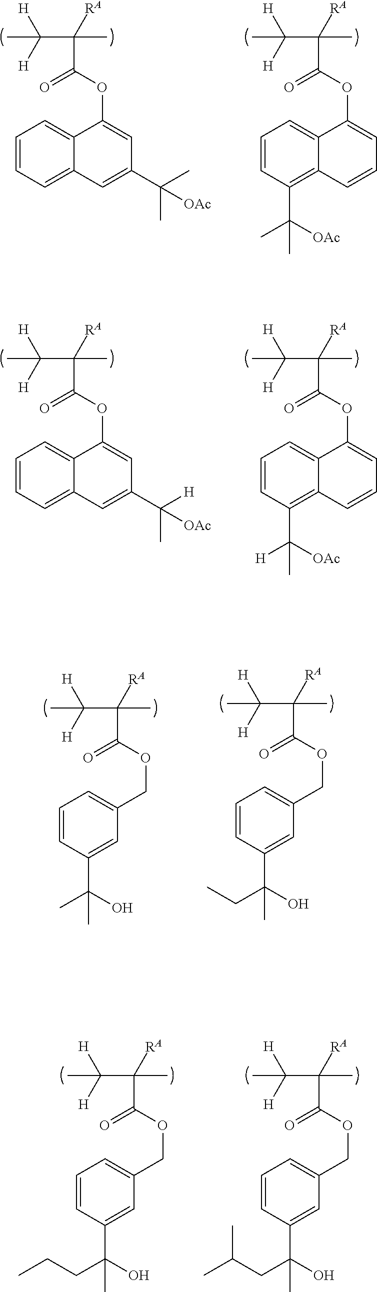

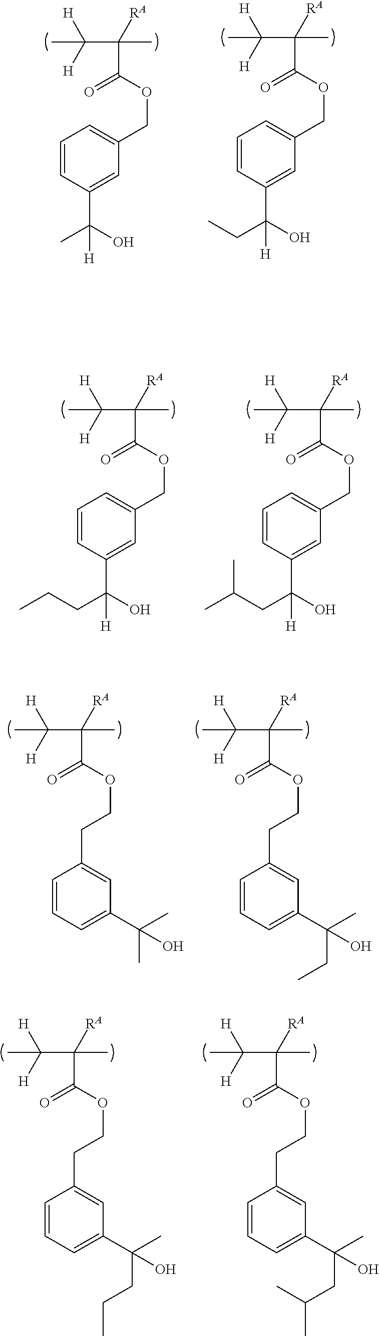

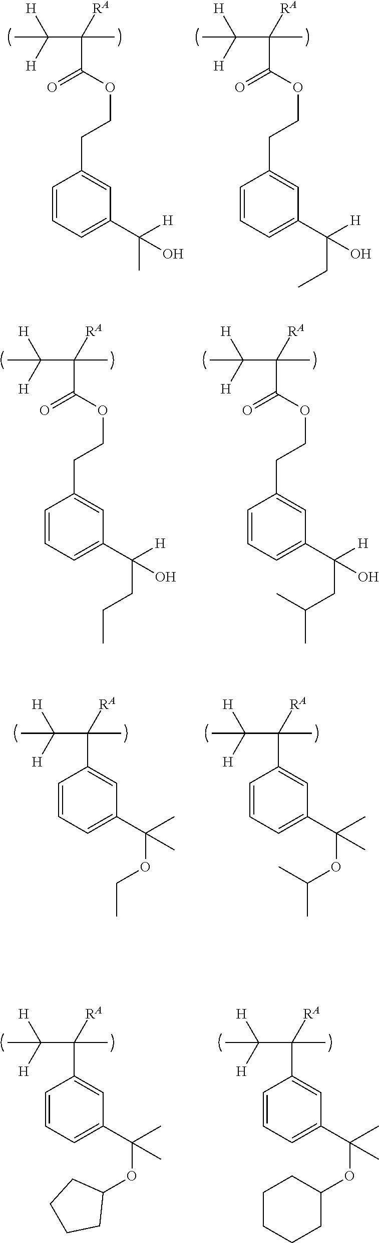

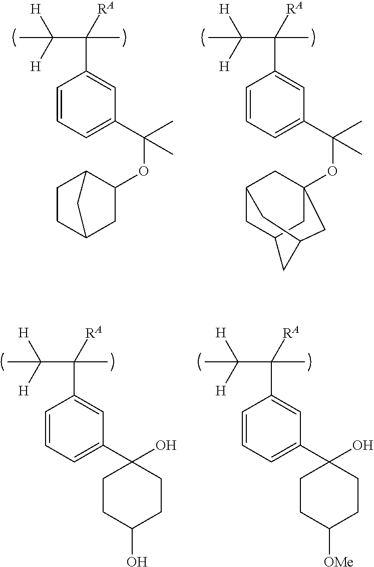

The base polymer preferably contains at least one repeat unit selected from a repeat unit having the formula (b1) (hereinafter also referred to as repeat unit (b1)) and a repeat unit having the formula (b2) (hereinafter also referred to as repeat unit (b2)).

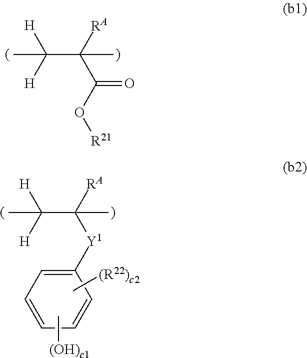

In the formulae (b1) and (b2), R^{A}'s are each independently a hydrogen atom, a fluorine atom, a methyl group, or a trifluoromethyl group, Y1 is a single bond or *—C(═O)—O—, * designates a point of attachment to the carbon atom in the backbone. R²¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—), R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, c1 is 1, 2, 3 or 4, and c2 is 0, 1, 2, 3, or 4, meeting 1≤c1+c2≤5.

Specific examples of the repeat unit (b1) are shown below, but not limited thereto. In the following formulae. R^{A} is as defined above.

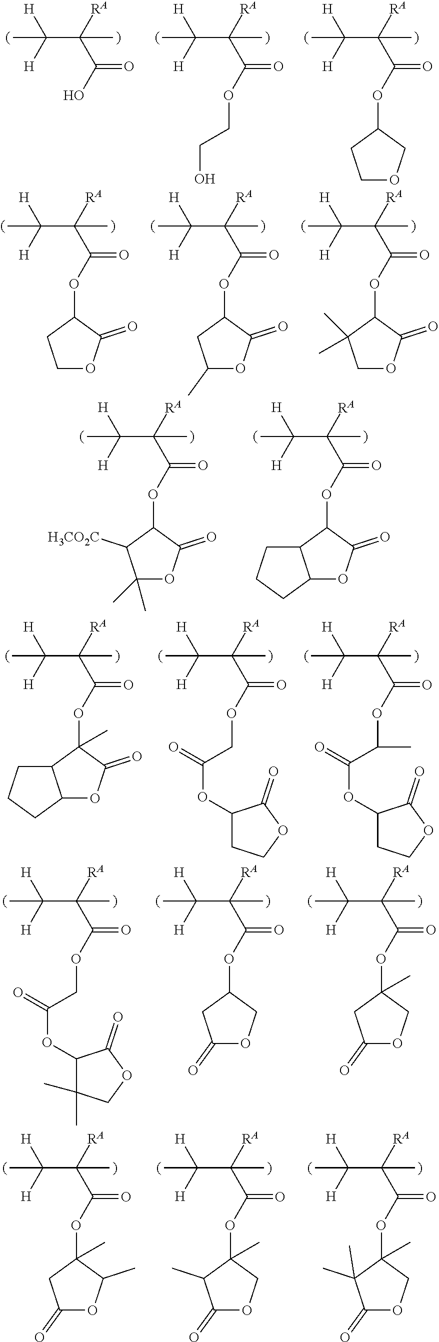

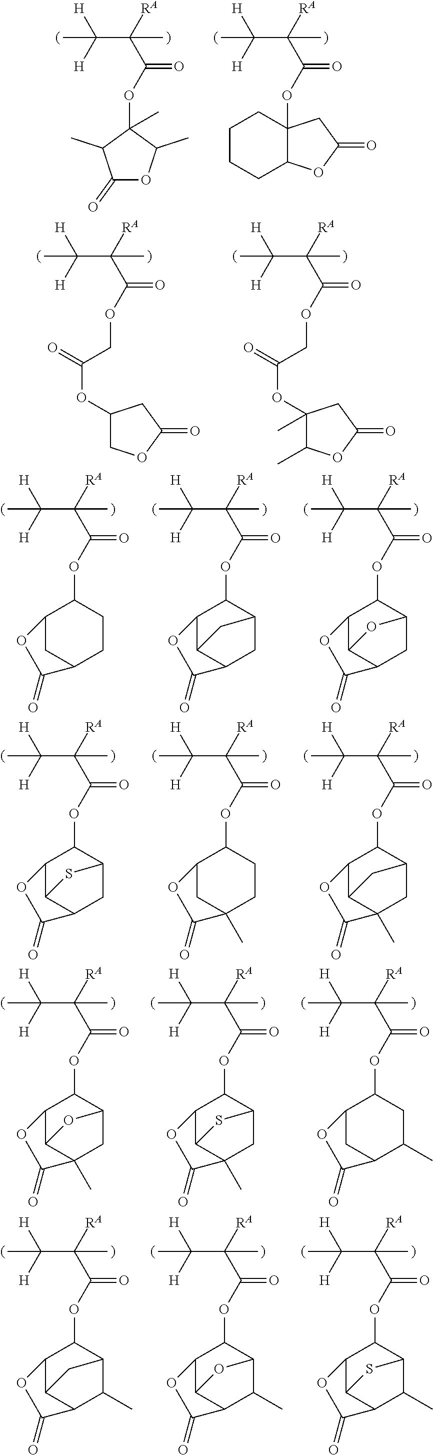

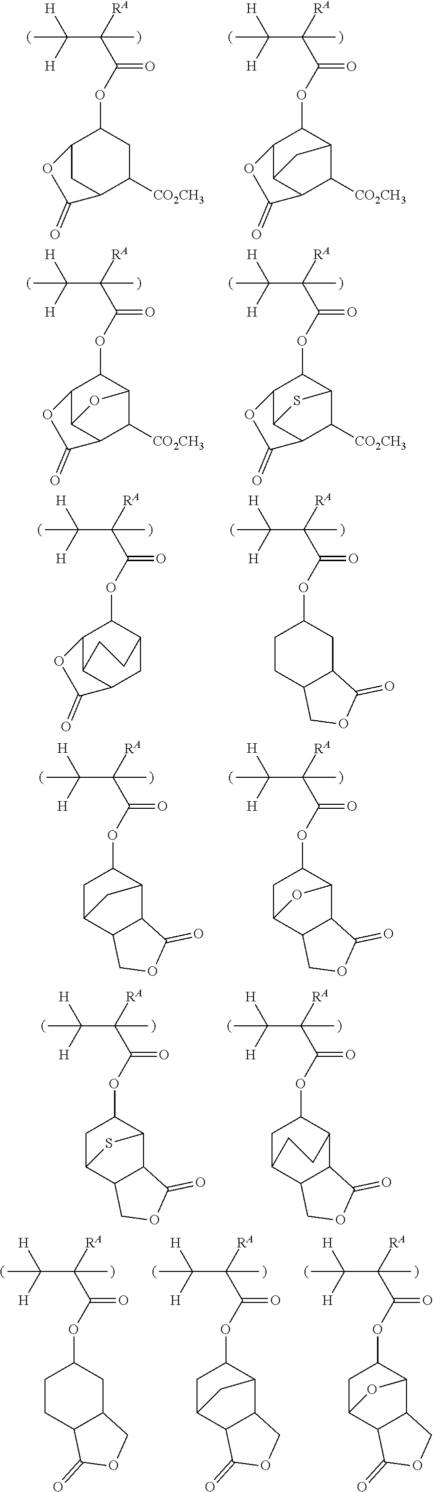

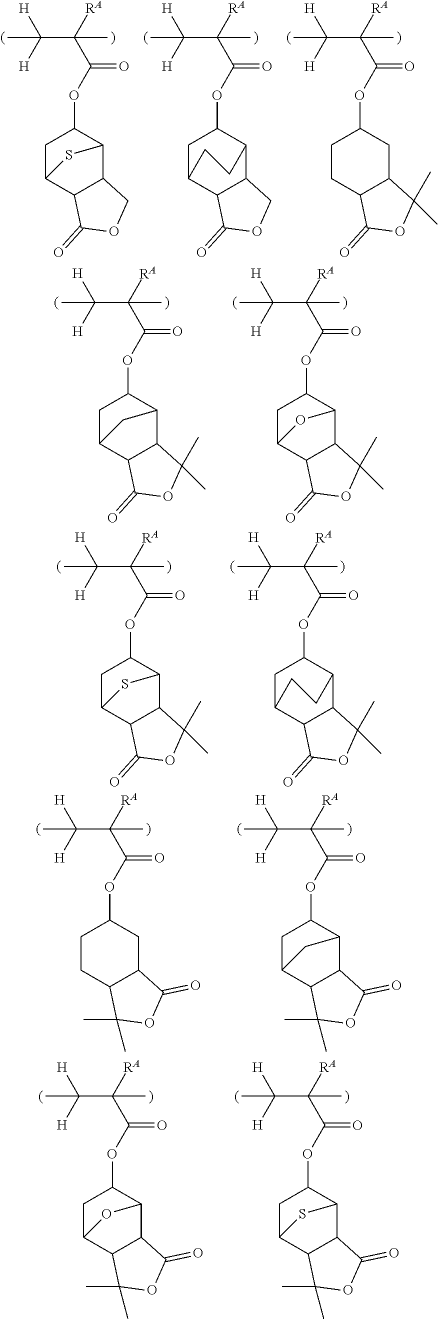

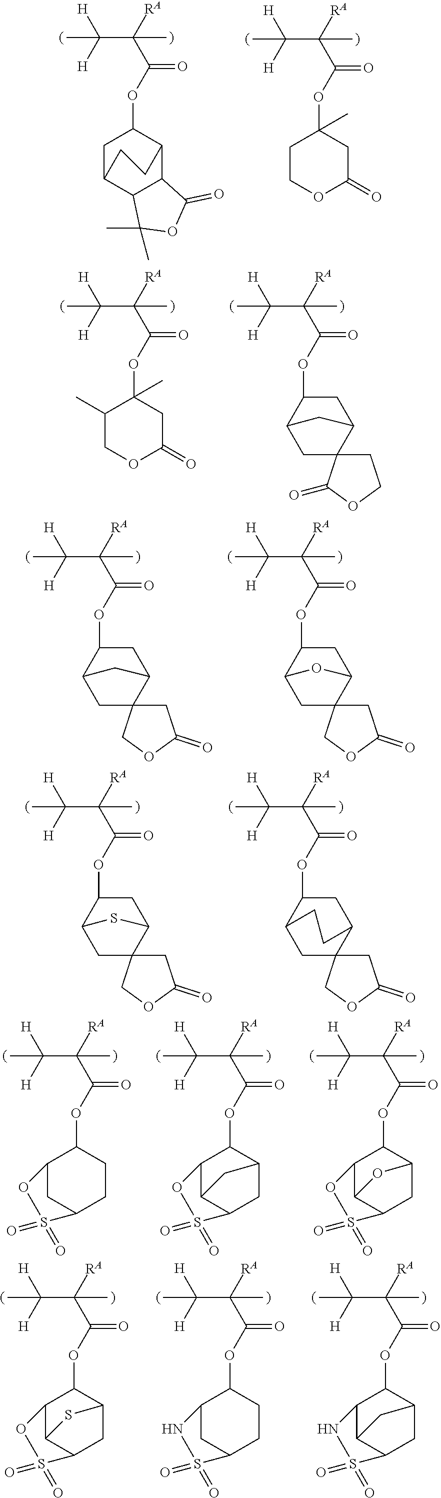

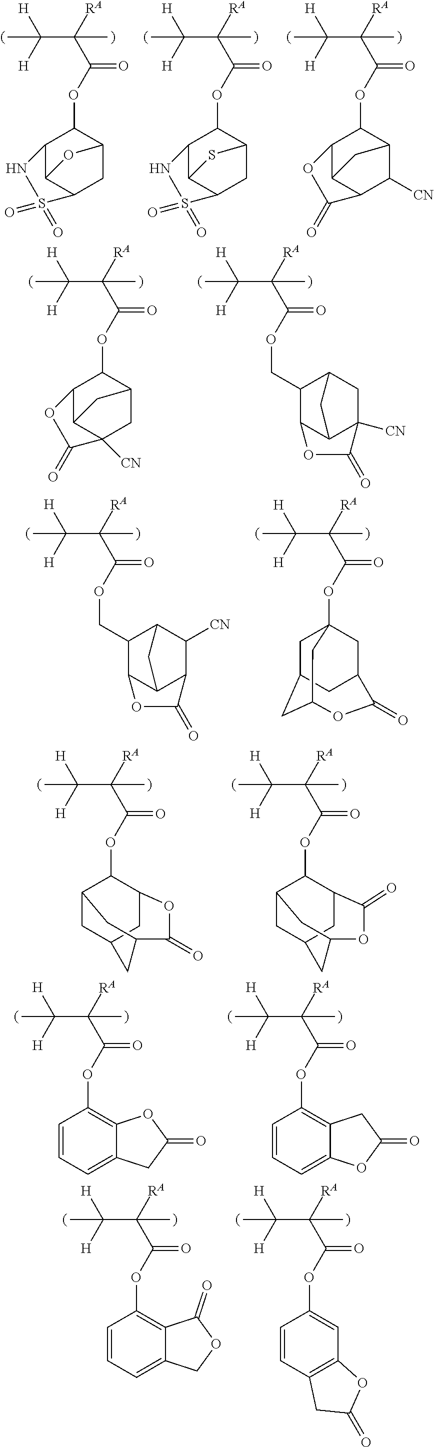

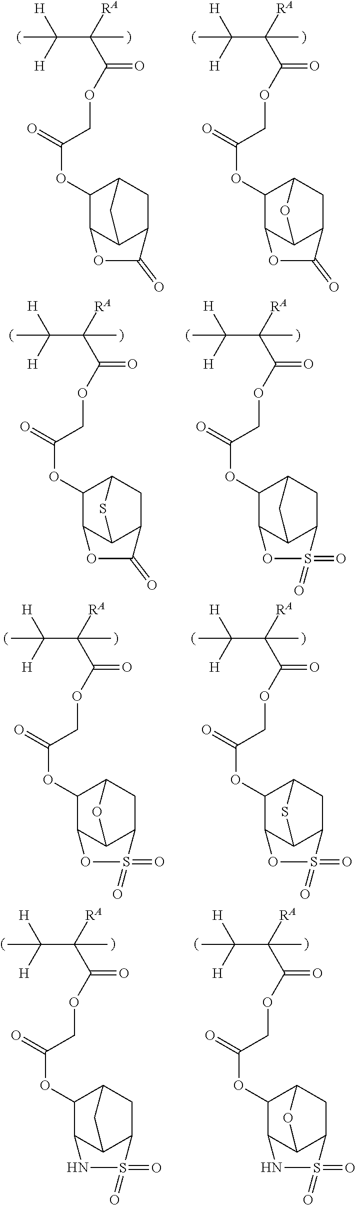

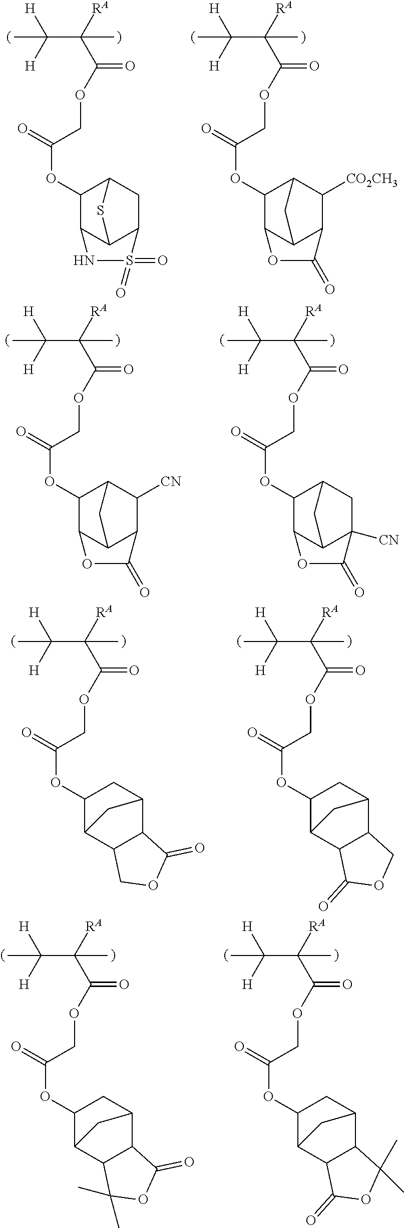

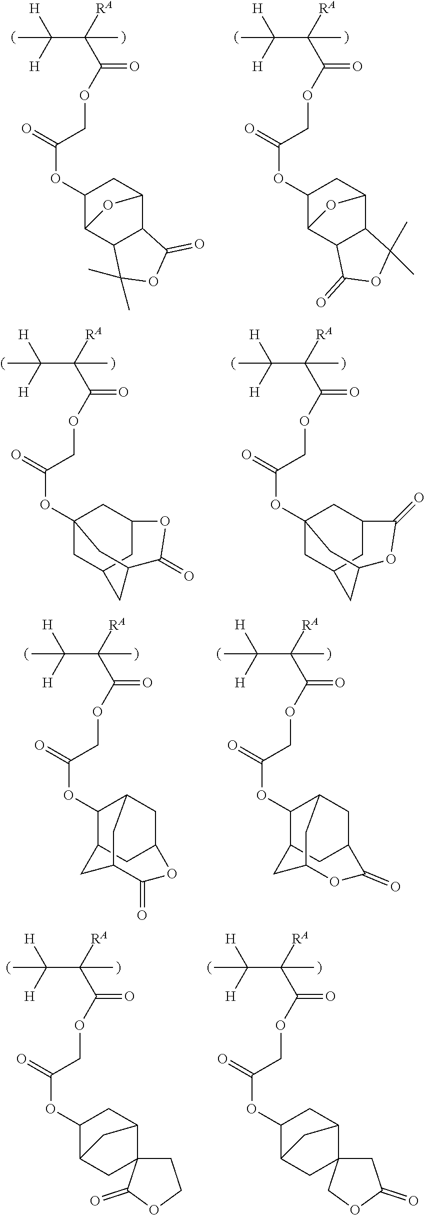

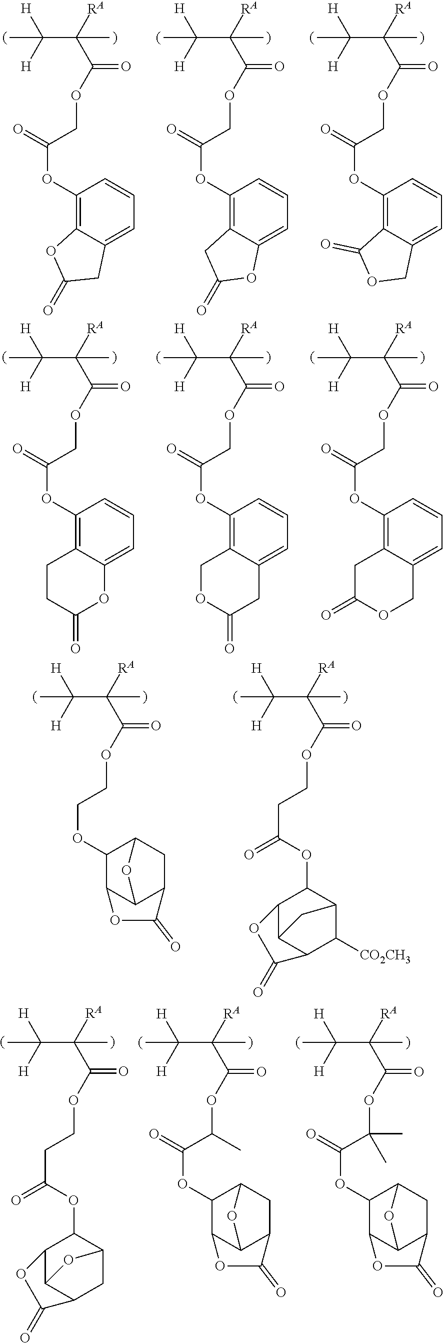

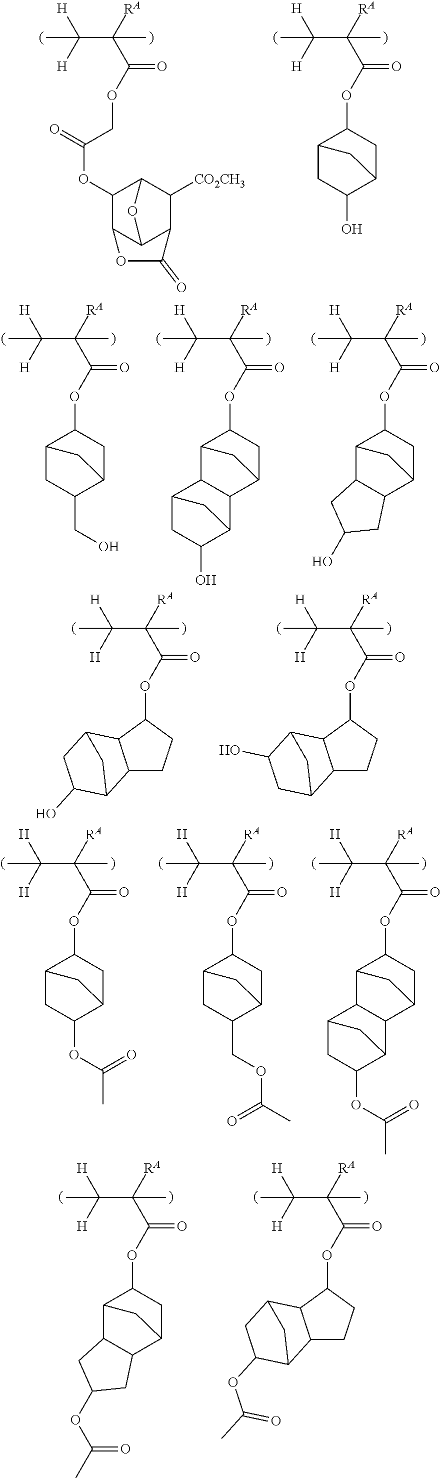

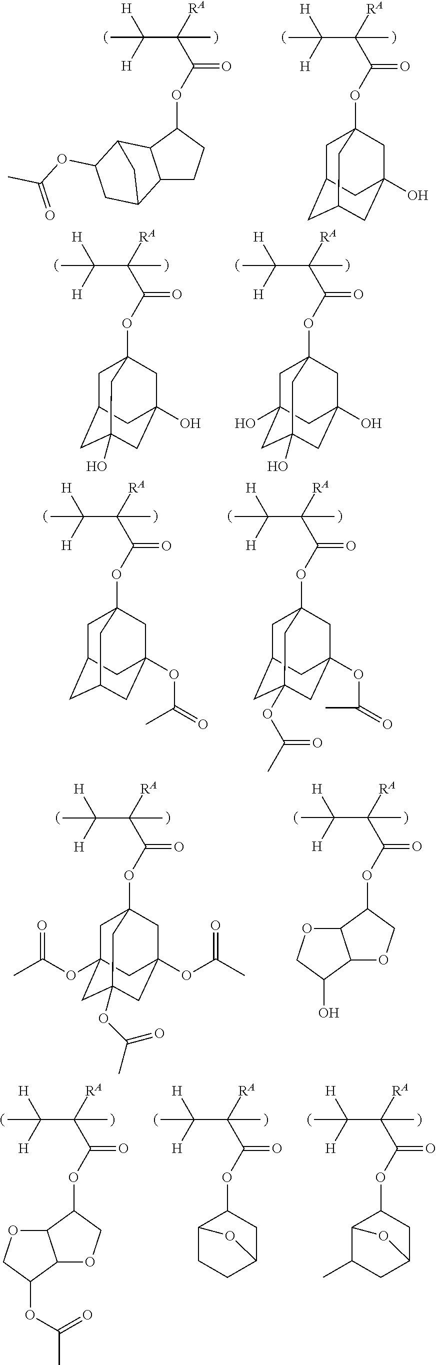

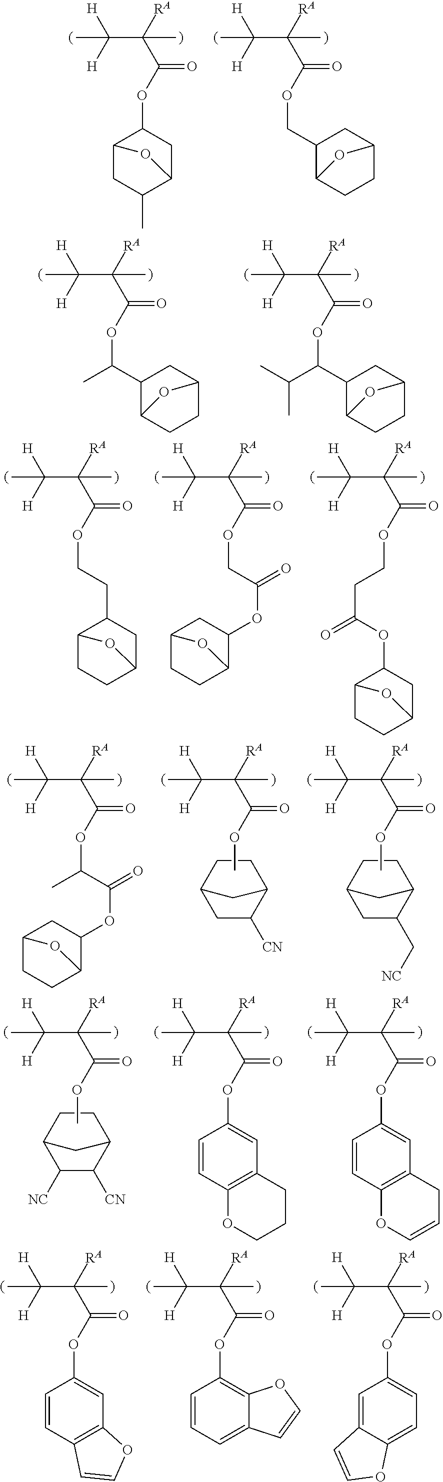

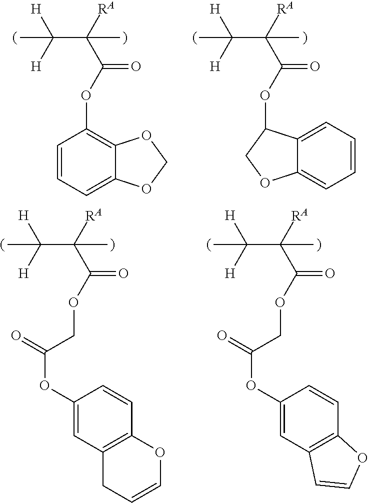

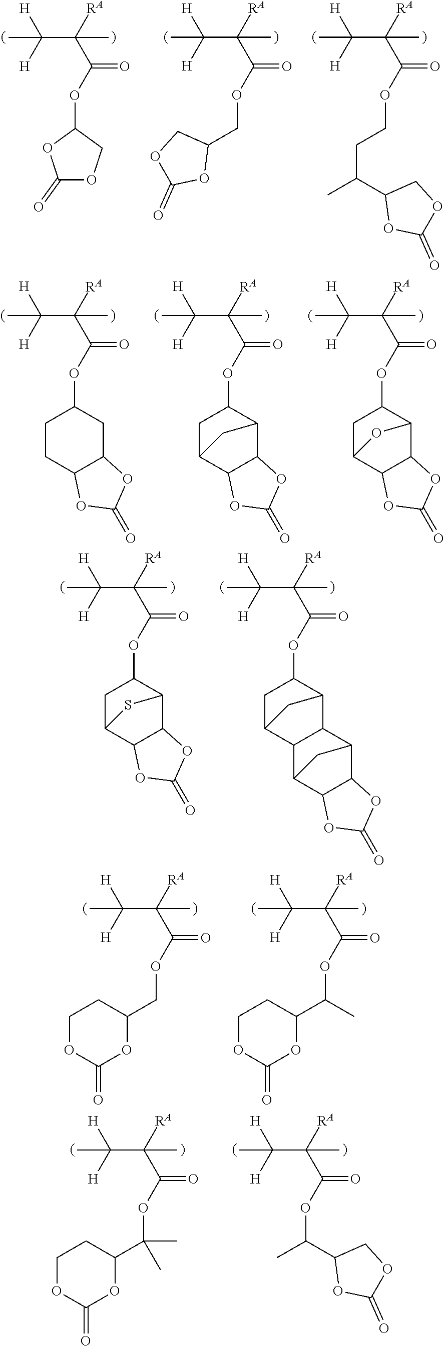

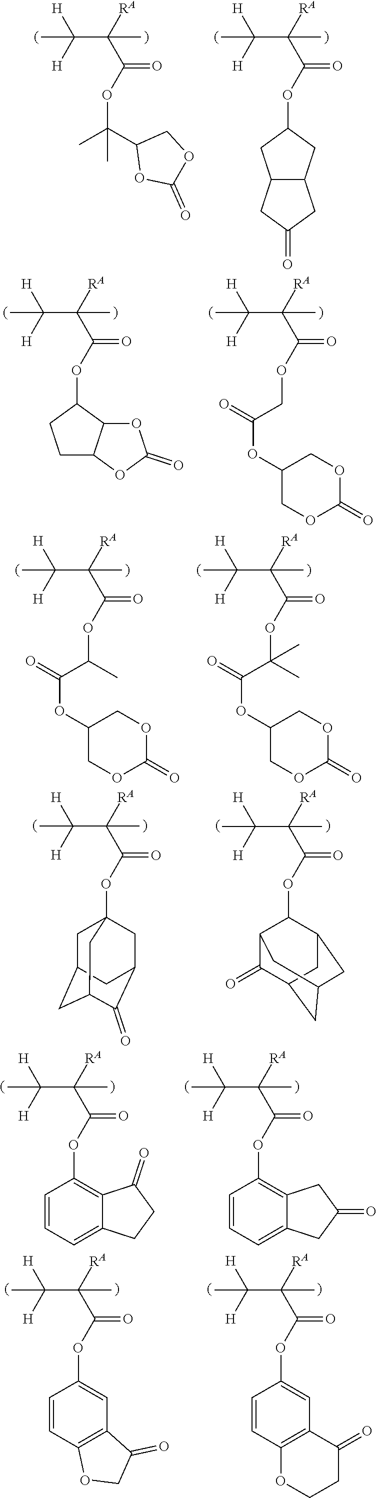

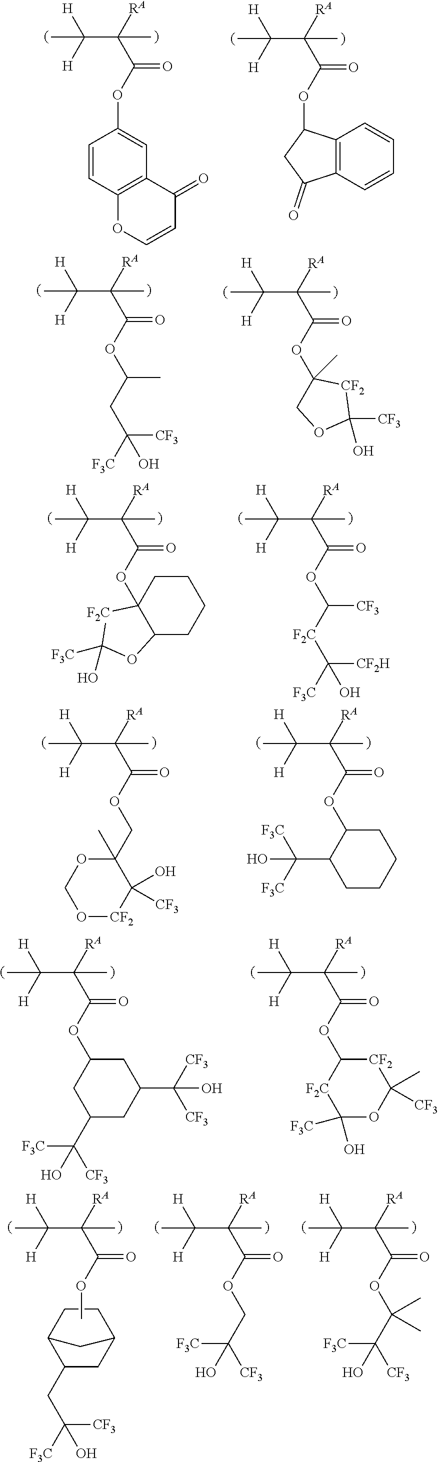

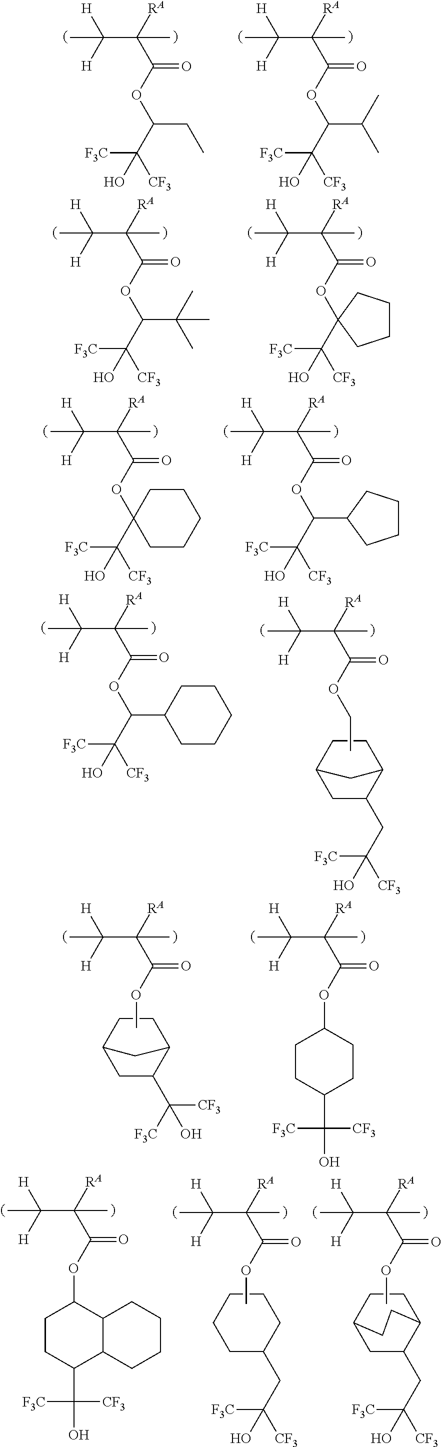

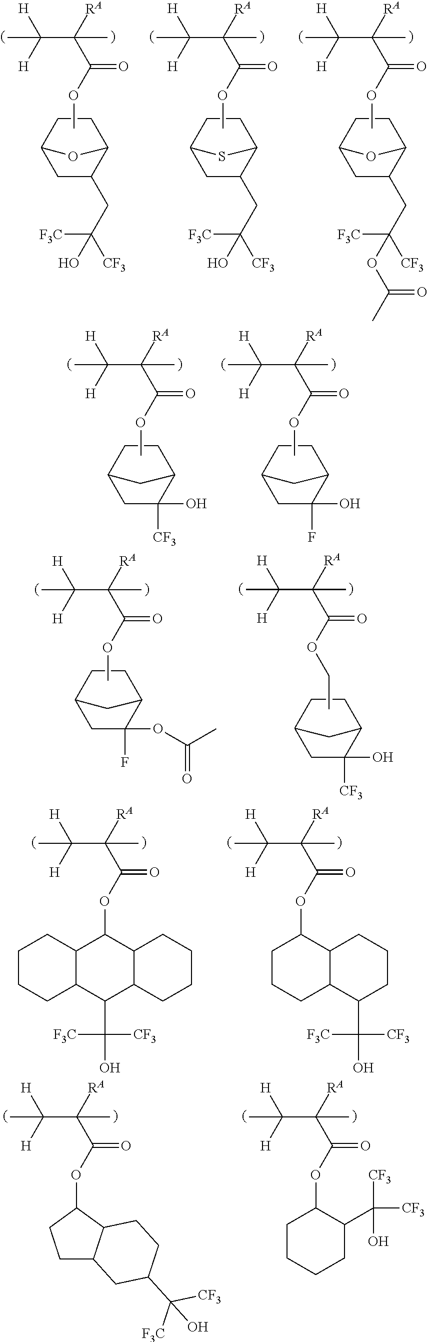

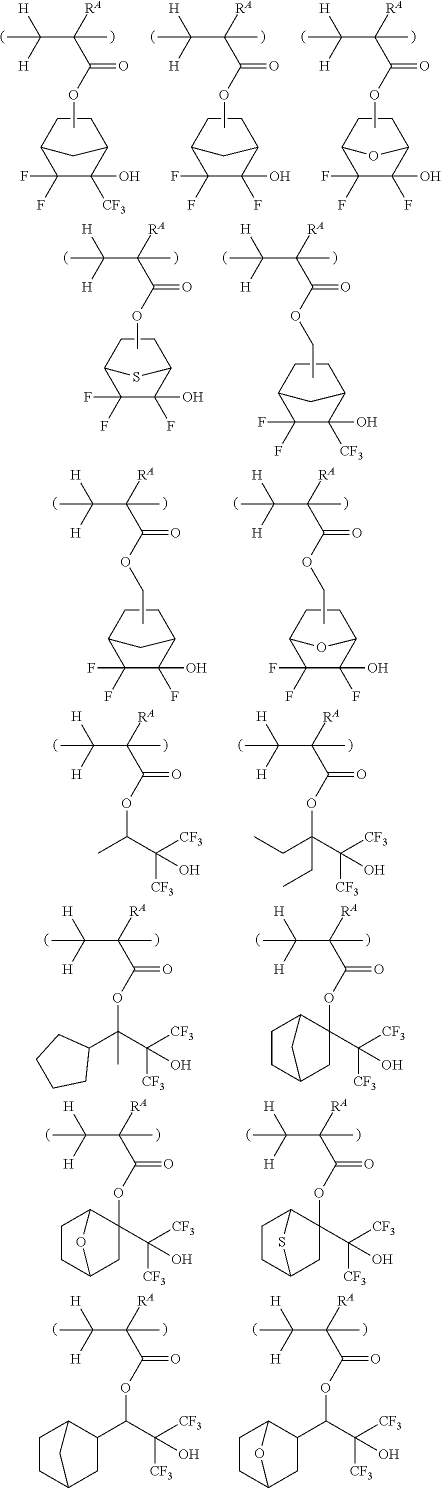

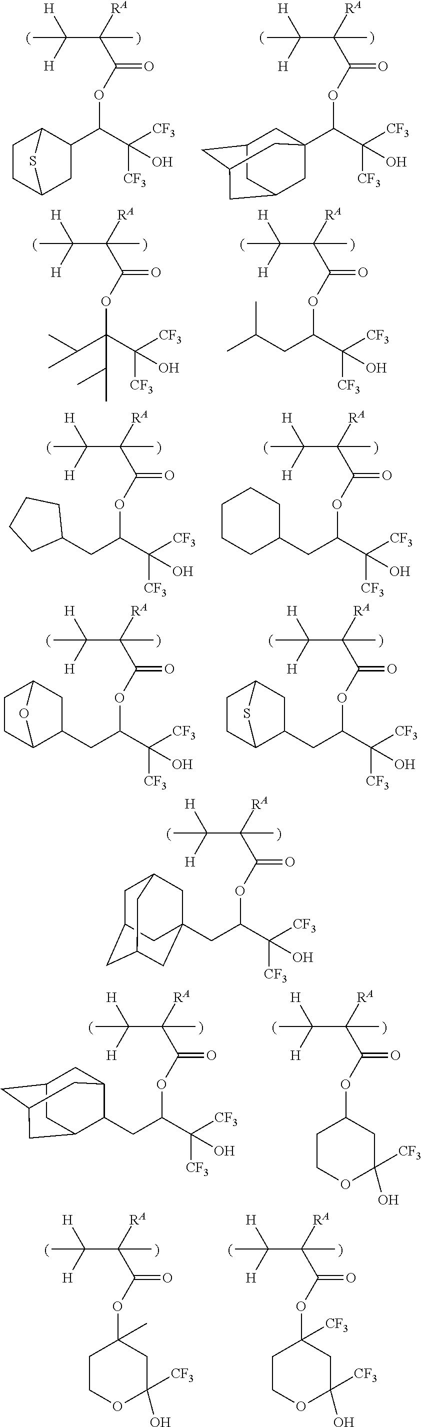

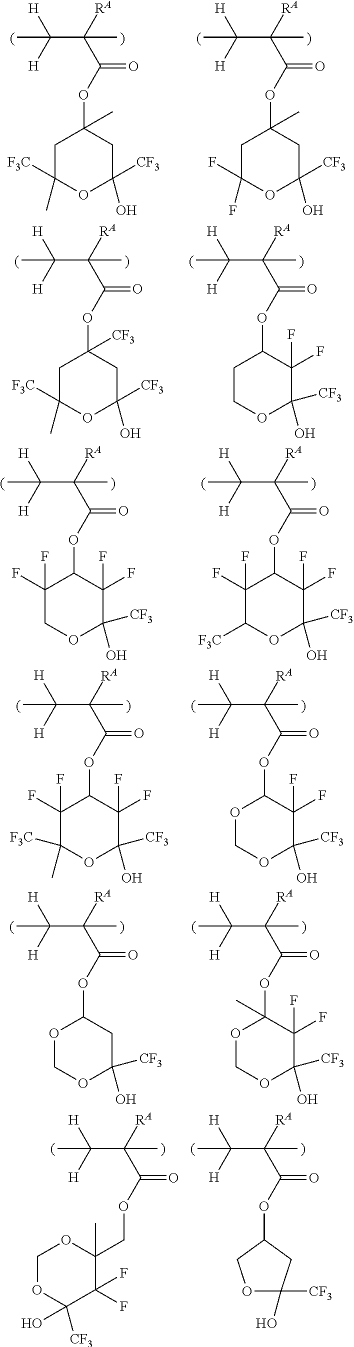

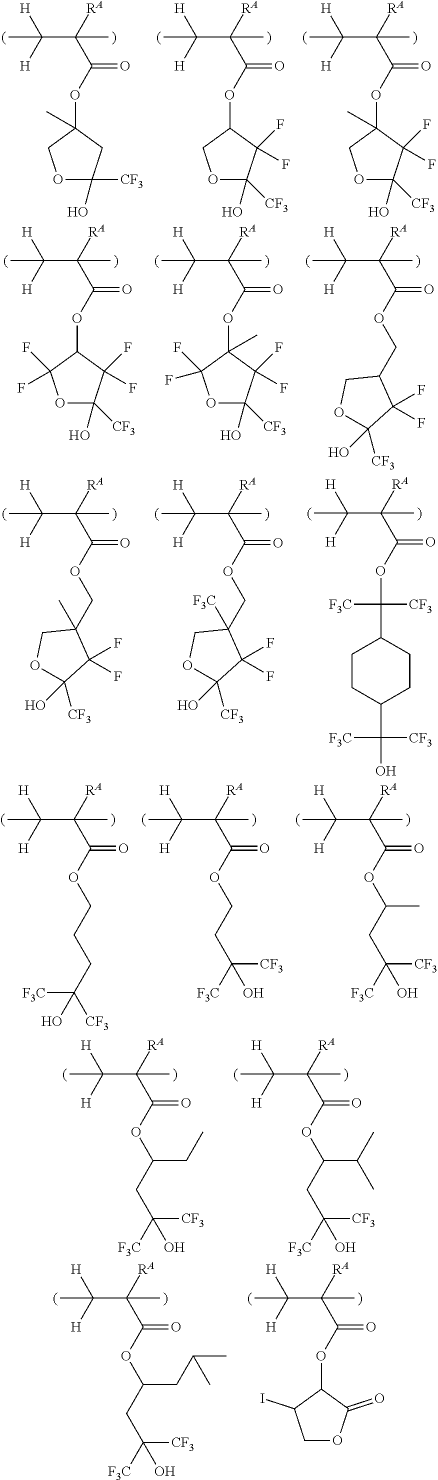

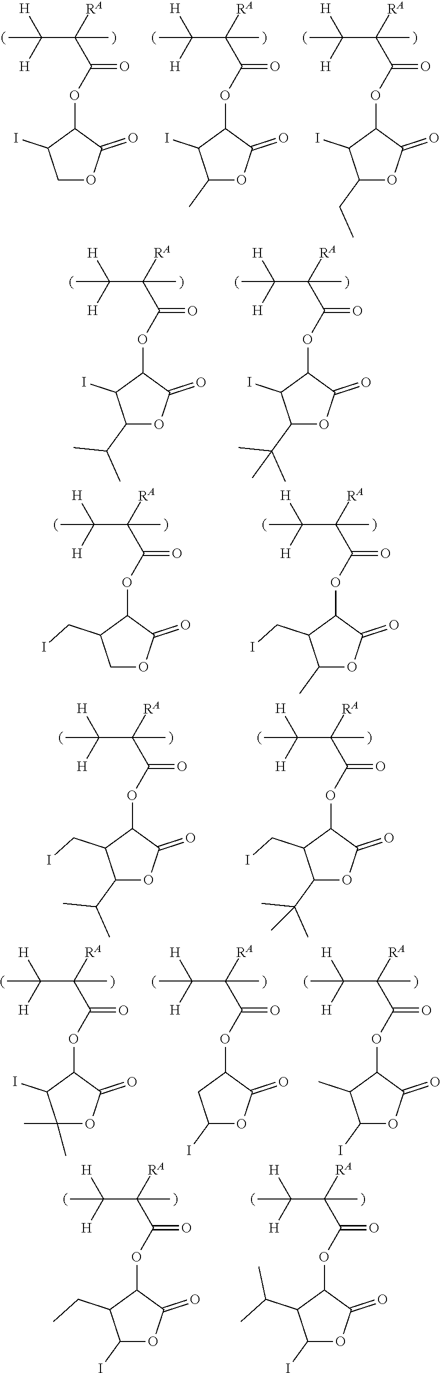

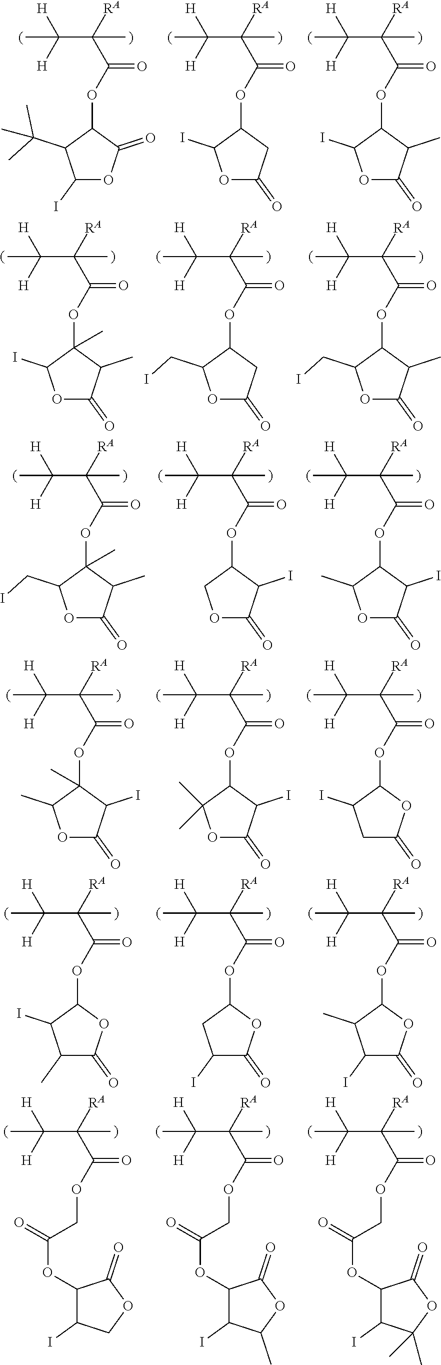

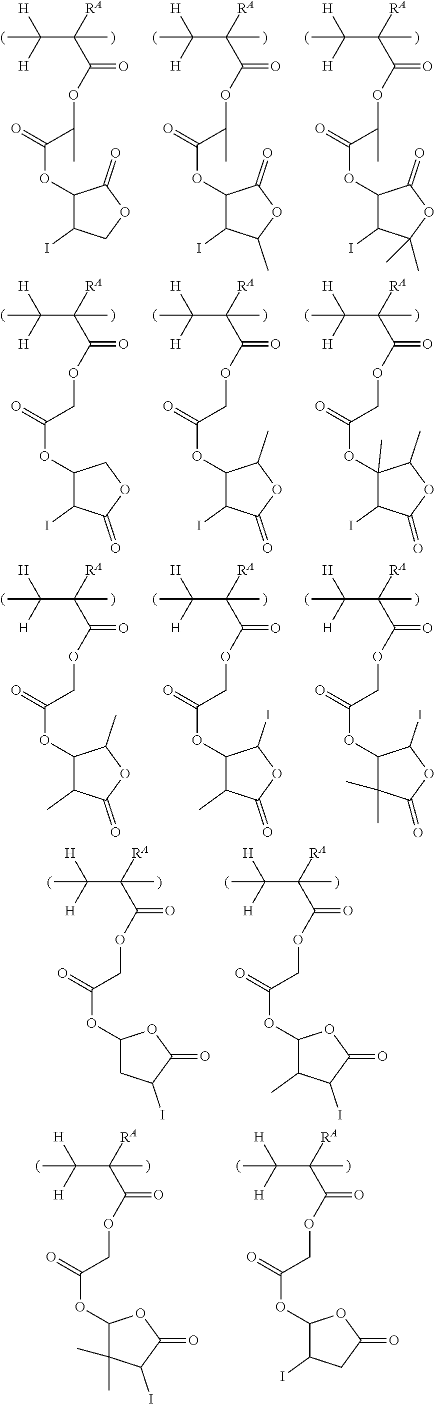

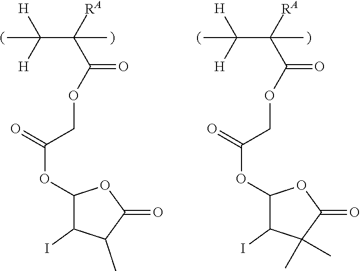

Specific examples of the repeat unit (b2) are shown below, but not limited thereto. In the following formulae. R^{A} is as defined above.

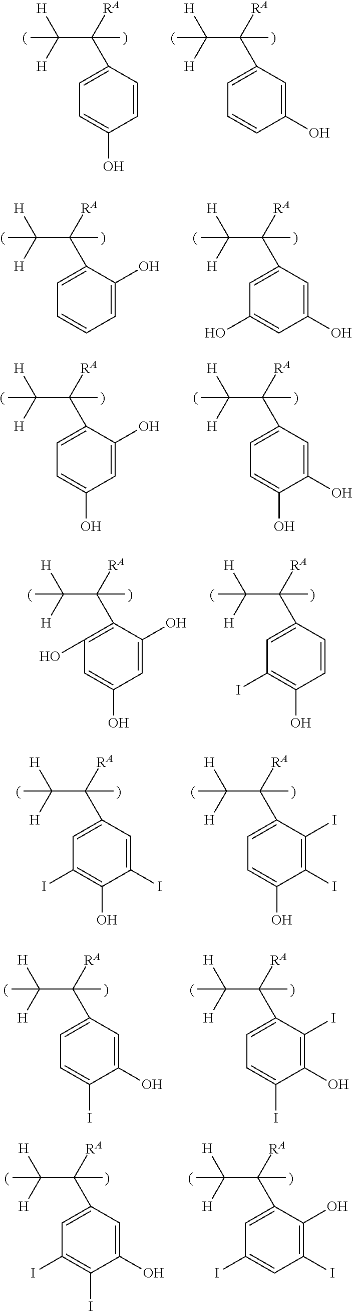

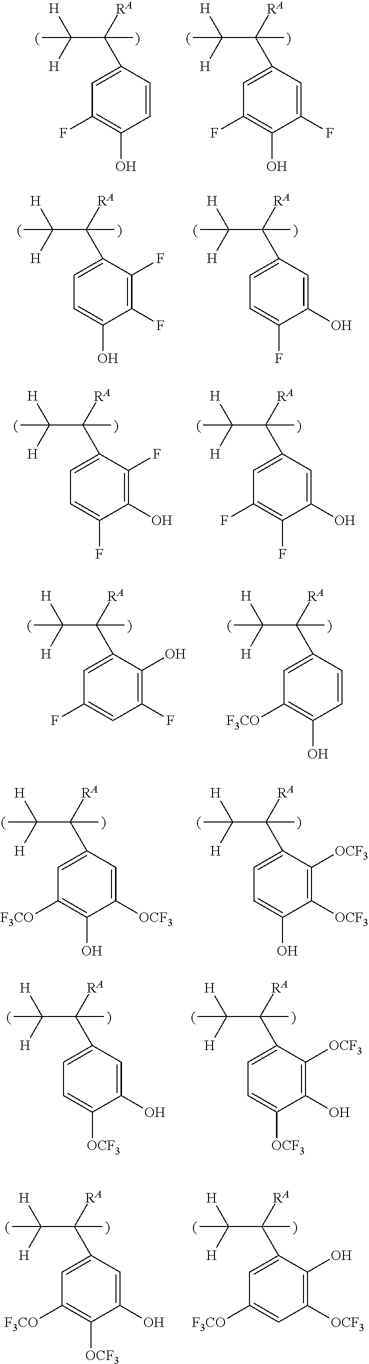

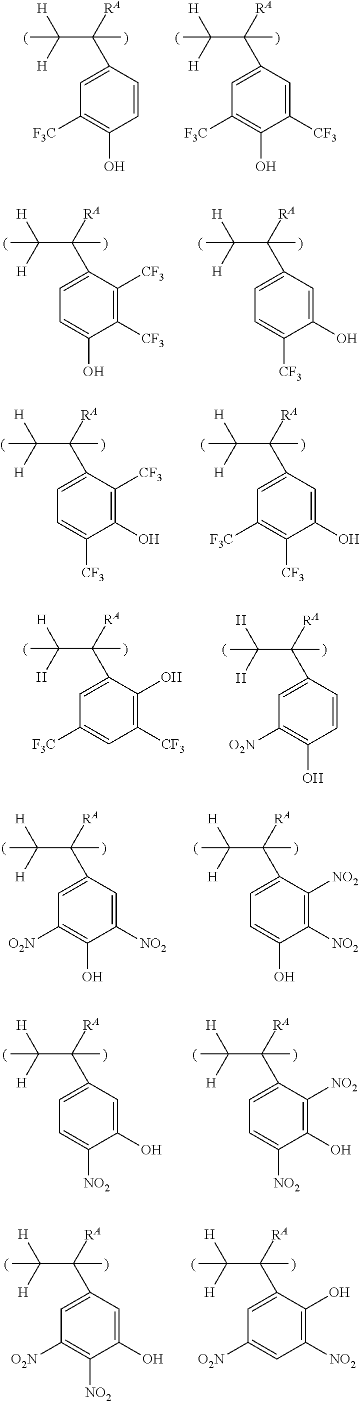

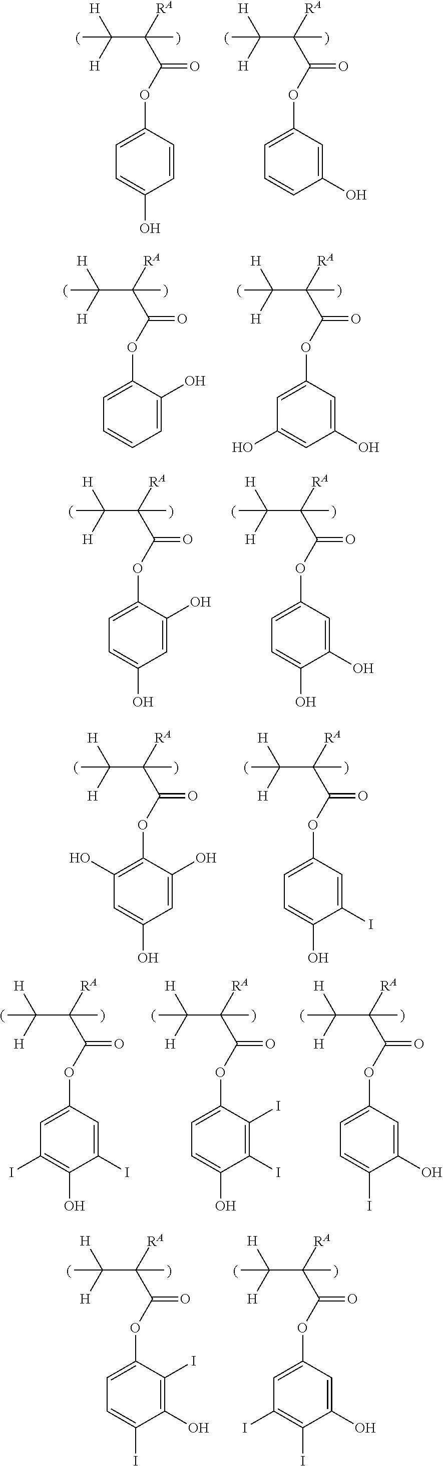

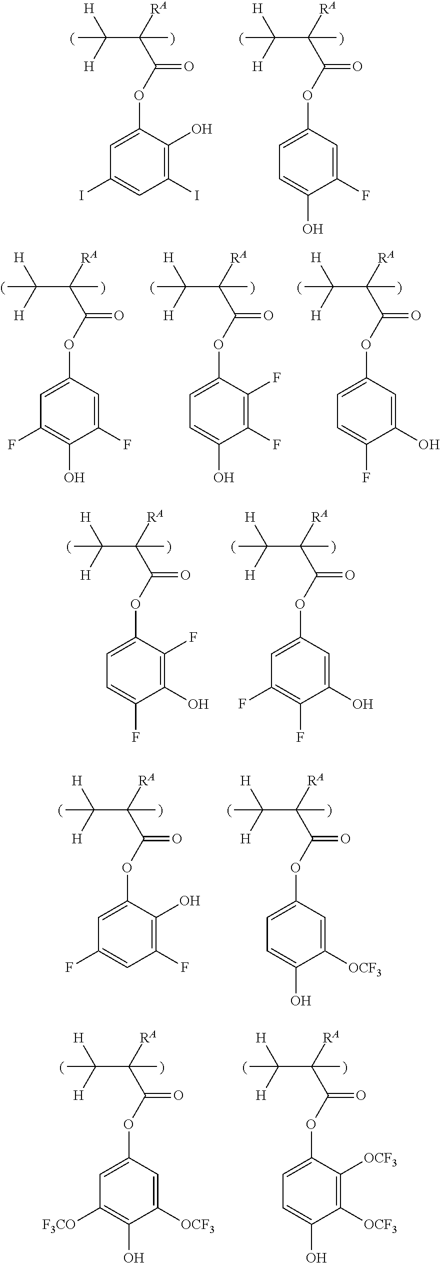

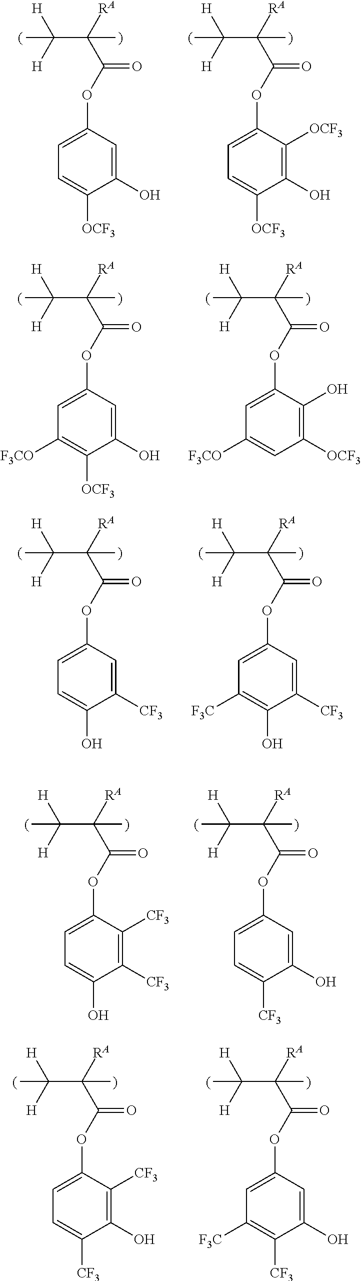

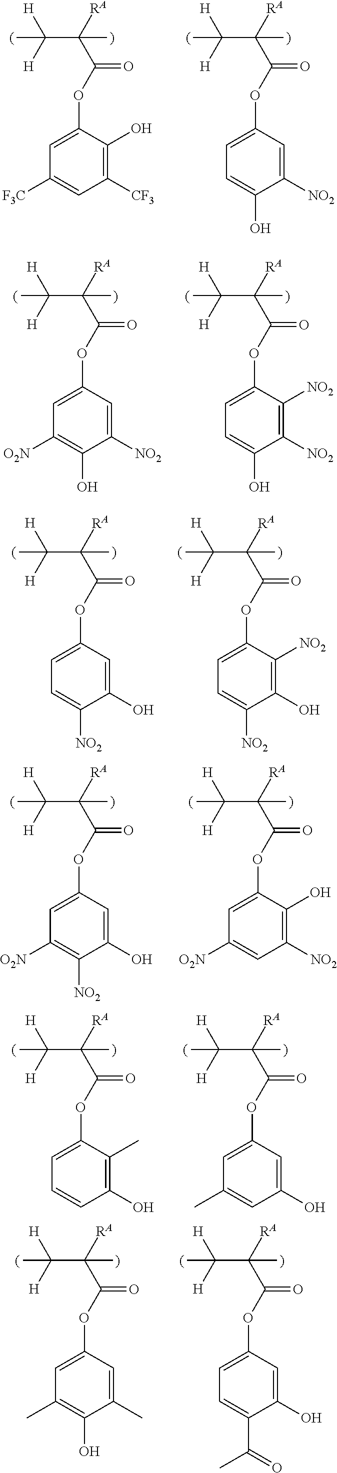

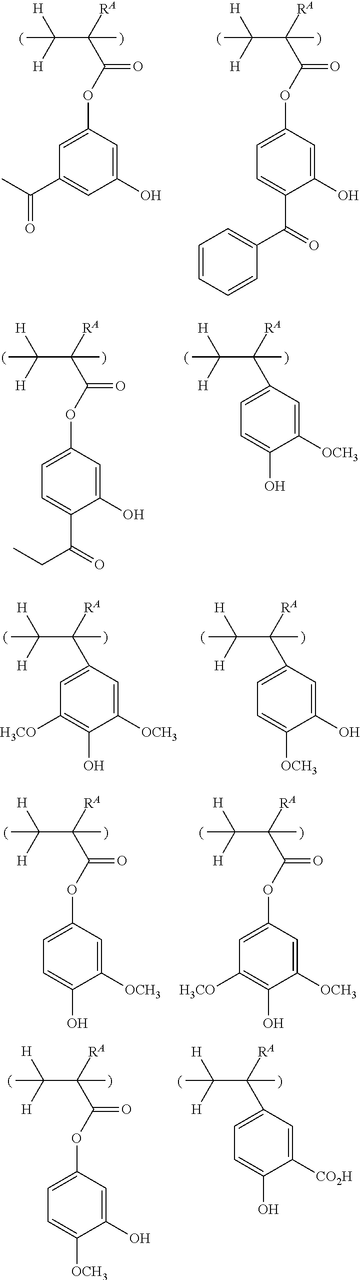

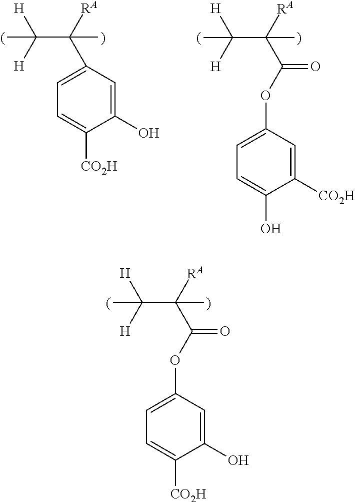

As the repeat unit (b1) or (b2), particularly a repeat unit having a lactone ring as a polar group is preferred in the case of ArF lithography, and a repeat unit having a phenol site is preferred in the case of KrF lithography, EB lithography, and EUV lithography.

The base polymer may contain at least one repeat unit selected from a repeat unit having the formula (c1) (hereinafter also referred to as repeat unit (c1)), a repeat unit having the formula (c2) (hereinafter also referred to as repeat unit (c2)), a repeat unit having the formula (c3) (hereinafter also referred to as repeat unit (c3)), a repeat unit having the formula (c4) (hereinafter also referred to as repeat unit (c4)), and a repeat unit having the formula (c5) (hereinafter also referred to as repeat unit (c5)).

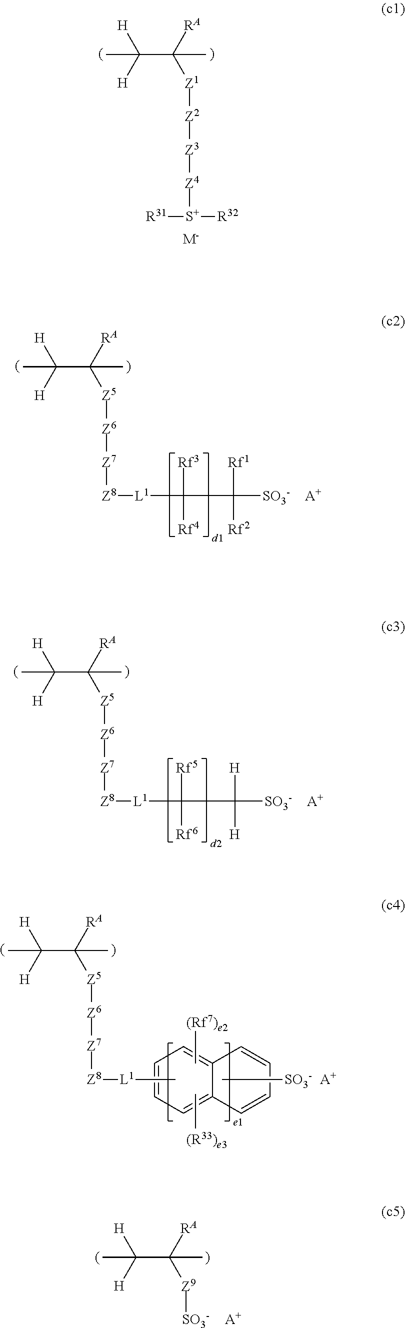

In the formulae (c1) to (c5), R^{A}'s are each independently a hydrogen atom, a fluorine atom, a methyl group, or a trifluoromethyl group, Z¹ is a single bond or a phenylene group which may have a substituent, Z² is a single bond, **—C(═O)—O—Z²¹—, **—C(═O)—NH—Z²¹—, or **—O—Z²¹., Z²¹ is a C₁-C₆ aliphatic hydrocarbylene group, a phenylene group, or a divalent group obtained by combining these groups, and may contain a halogen atom, a carbonyl group, an ester bond, an ether bond, or a hydroxy group, Z³ is a single bond, an ether bond, an ester bond, a sulfonate ester bond, an amide bond, a sulfonamide bond, a carbonate bond, or a carbamate bond, Z⁴ is a single bond, or a C₁-C₆ aliphatic hydrocarbylene group, a phenylene group, or a divalent group obtained by combining these groups, and may contain a halogen atom, a carbonyl group, an ester bond, an ether bond, or a hydroxy group, Z⁵'s are each independently a single bond, a phenylene group which may have a substituent, a naphthylene group, or *—C(═O)—O—Z⁵¹—, Z⁵¹ is a C₁-C₁₀ aliphatic hydrocarbylene group, a phenylene group, or a naphthylene group, and the aliphatic hydrocarbylene group may contain a halogen atom, a hydroxy group, an ether bond, an ester bond, or a lactone ring,

Z′ is a single bond, an ether bond, an ester bond, a sulfonate ester bond, an amide bond, a sulfonamide bond, a carbonate bond, or a carbamate bond, Z⁷'s are each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom, Z⁸'s are each independently a single bond, ****—Z⁸¹—C(═O)—O—, ****—C(═O)—NH—Z⁸¹—, or ****—O—Z⁸¹—, Z⁸¹ is a C₁-C₂₀ hydrocarbylene group which may contain a heteroatom, Z⁹ is a single bond, a methylene group, an ethylene group, a phenylene group, a fluorinated phenylene group, a phenylene group substituted with a trifluoromethyl group, *—C(═O)—O—Z°1—, *—C(═O)—N(H)—Z⁹¹—, or *—O—Z⁹¹—, Z°1 is a C₁-C₆ aliphatic hydrocarbylene group, a phenylene group, a fluorinated phenylene group, or a phenylene group substituted with a trifluoromethyl group, and may contain a carbonyl group, an ester bond, an ether bond, or a hydroxy group, * designates a point of attachment to the carbon atom in the backbone. ** designates a point of attachment to Z¹, *** designates a point of attachment to Z⁶, and **** designates a point of attachment to Z⁷.

The aliphatic hydrocarbylene group of Z²¹, z⁵¹, and Z⁹¹ may be straight, branched, or cyclic, and specific examples thereof include alkanediyl groups such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,1-diyl group, a butane-1,2-diyl group, a butane-1,3-diyl group, a butane-2,3-diyl group, a butane-1,4-diyl group, a 1,1-dimethylethane-1,2-diyl group, a pentane-1,5-diyl group, a 2-methylbutane-1,2-diyl group, and a hexane-1,6-diyl group; cycloalkanediyl groups such as a cyclopropanediyl group, a cyclobutanediyl group, a cyclopentanediyl group, and a cyclohexanediyl group; and a group obtained by combining these groups.

The hydrocarbylene group which may contain a heteroatom of Z⁷¹ and Z⁸¹ may be saturated or unsaturated and straight, branched, or cyclic. Specific examples thereof are shown below, but not limited thereto.

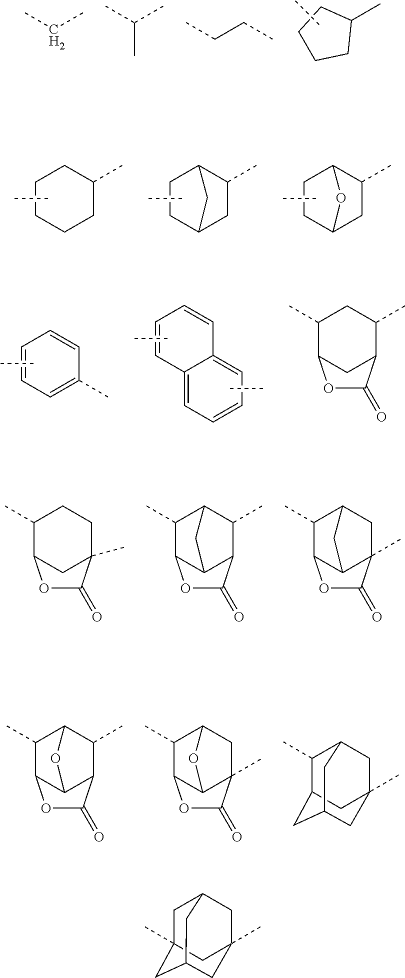

In the formulae, a broken line designates a point of attachment.

In the formula (c1), R³¹ and R³² are each independently a C₁-C₂₀ hydrocarbyl group which may contain a heteroatom, The hydrocarbyl group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, and a tert-butyl group; C₃-C₂₀ Cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group, a naphthyl group, and a thienyl group; C₇-C₂₀ aralkyl groups such as a benzyl group, a 1-phenylethyl group, and a 2-phenylethyl group; and a group obtained by combining these groups, and aryl groups are preferred. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

R³¹ and R³² may bond together to form a ring with the sulfur atom to which they are attached. At this time, specific examples of the ring include those having the following formulae.

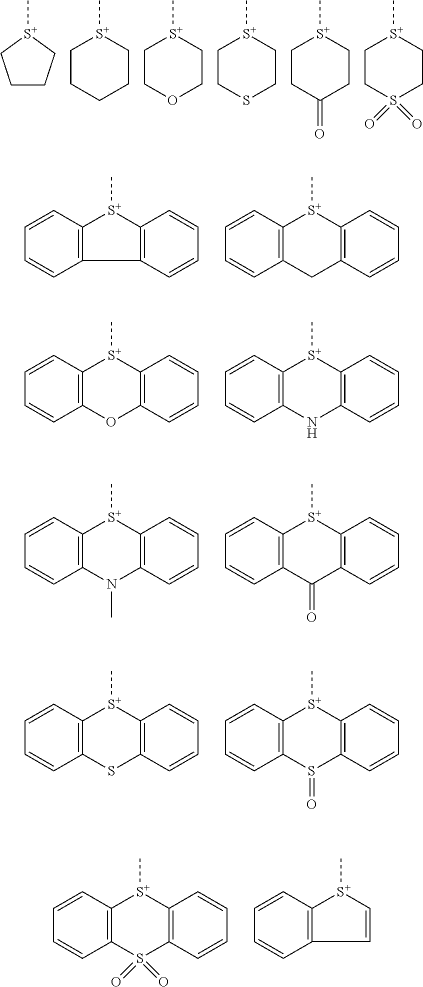

In the formulae, a broken line designates a point of attachment to Z⁴.

Specific examples of the cation of the repeat unit (c1) are shown below, but not limited thereto. In the following formulae. R^{A} is as defined above.

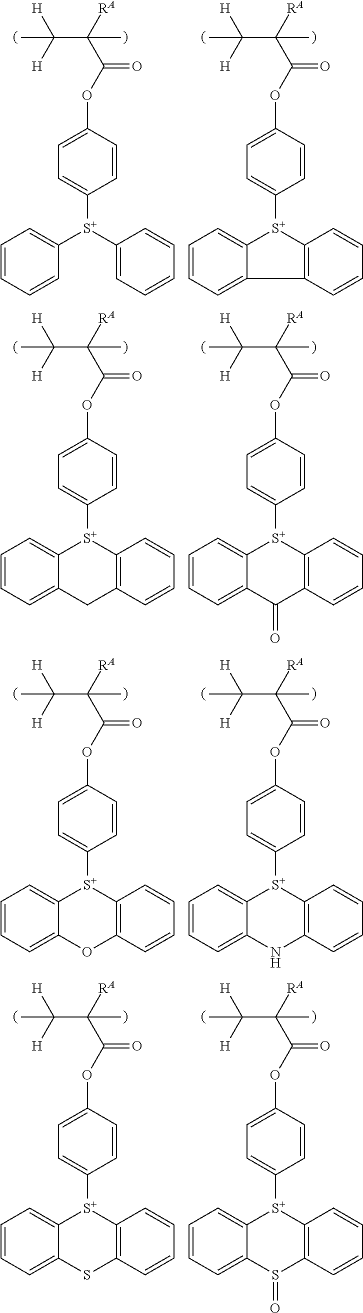

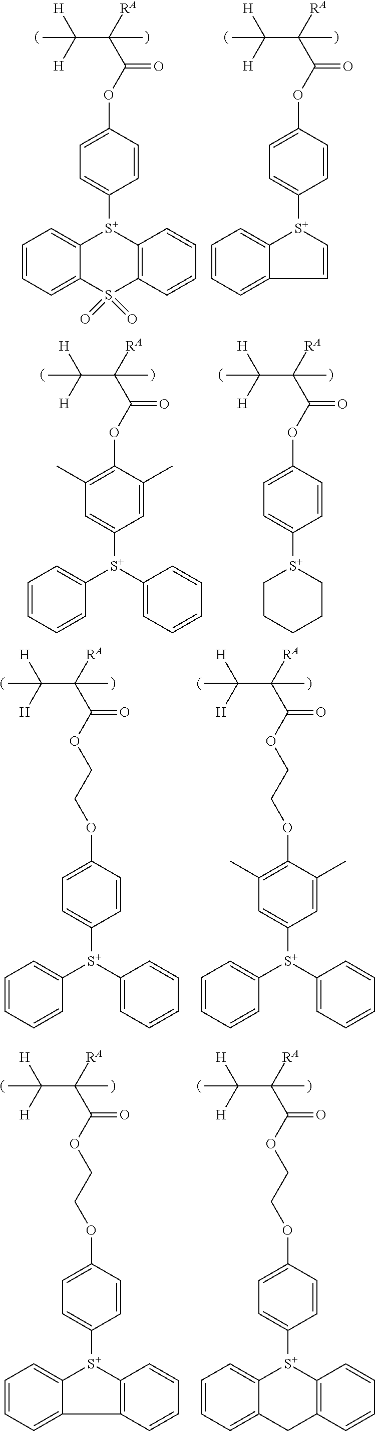

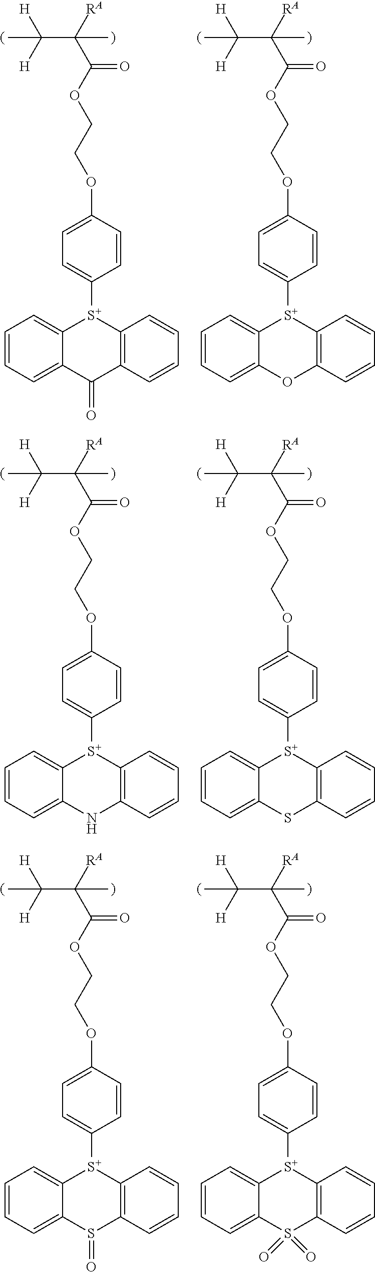

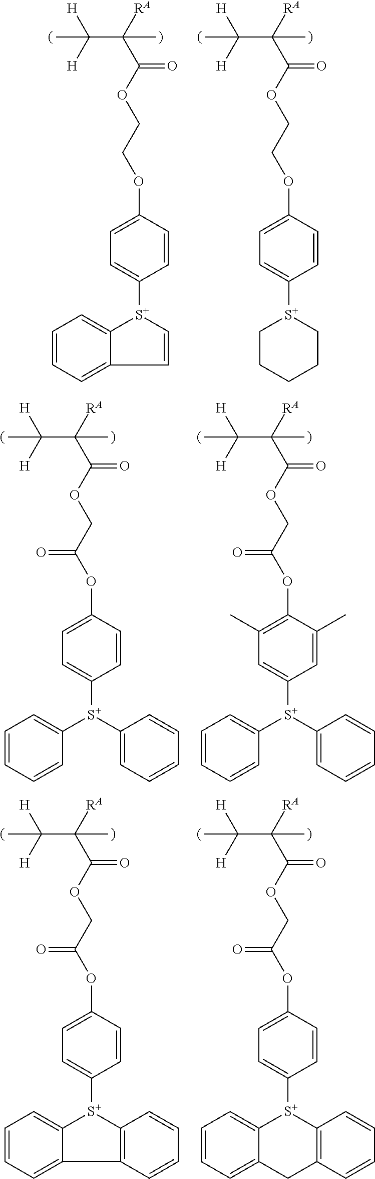

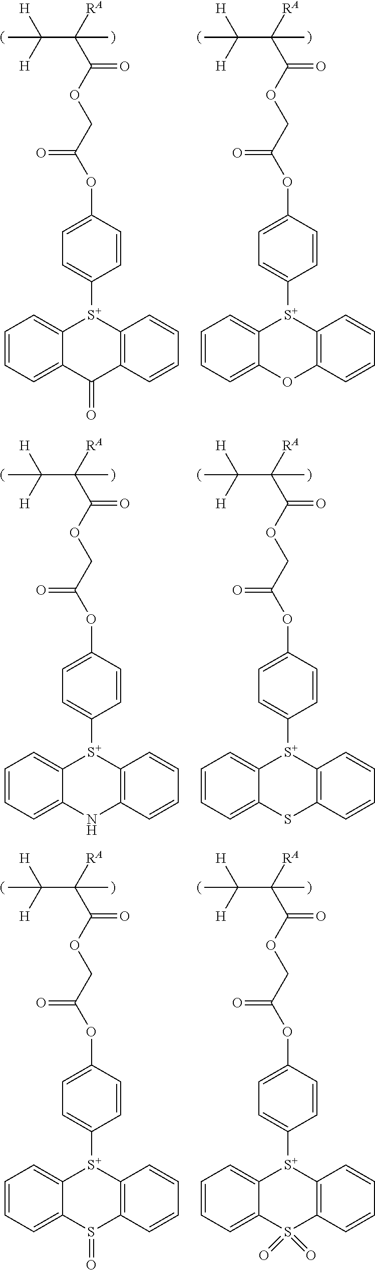

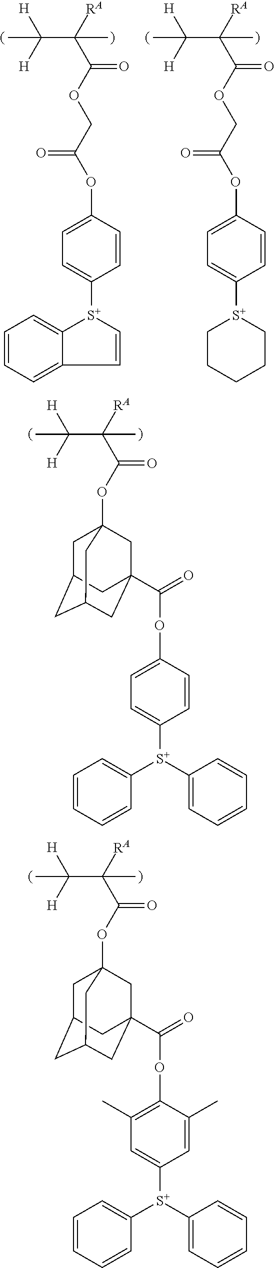

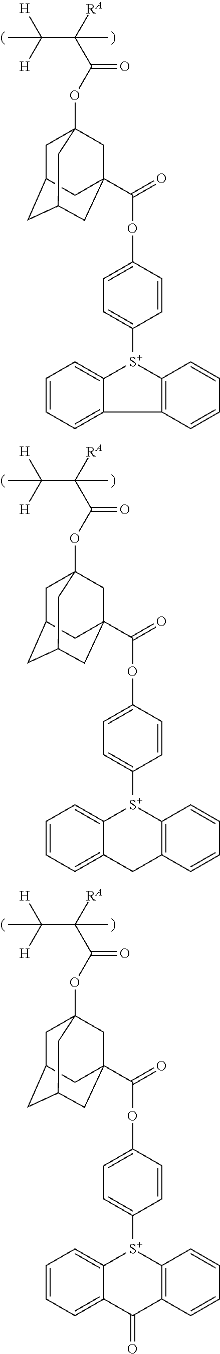

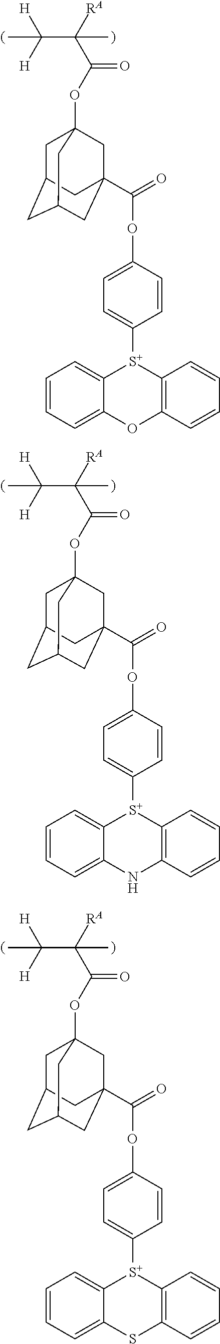

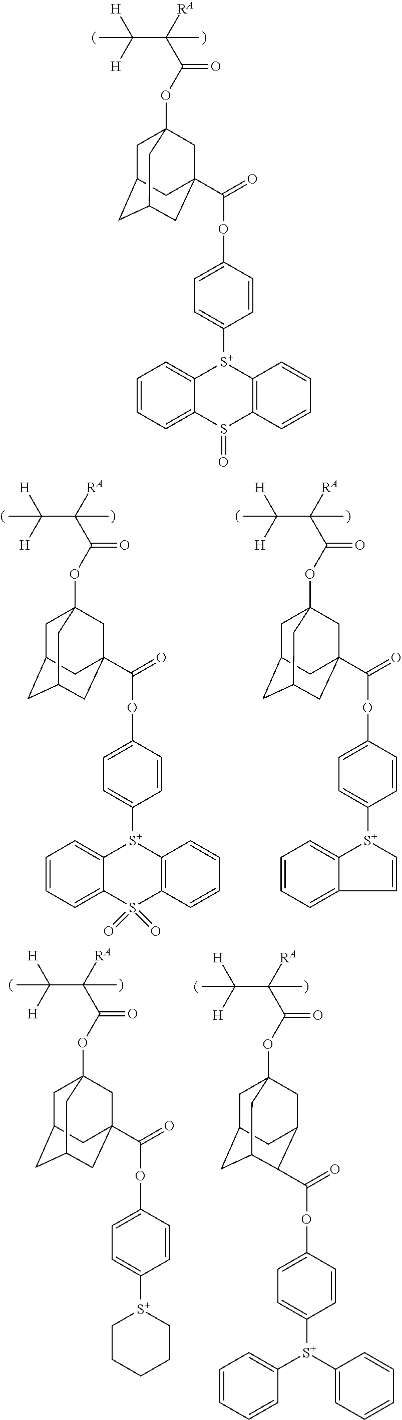

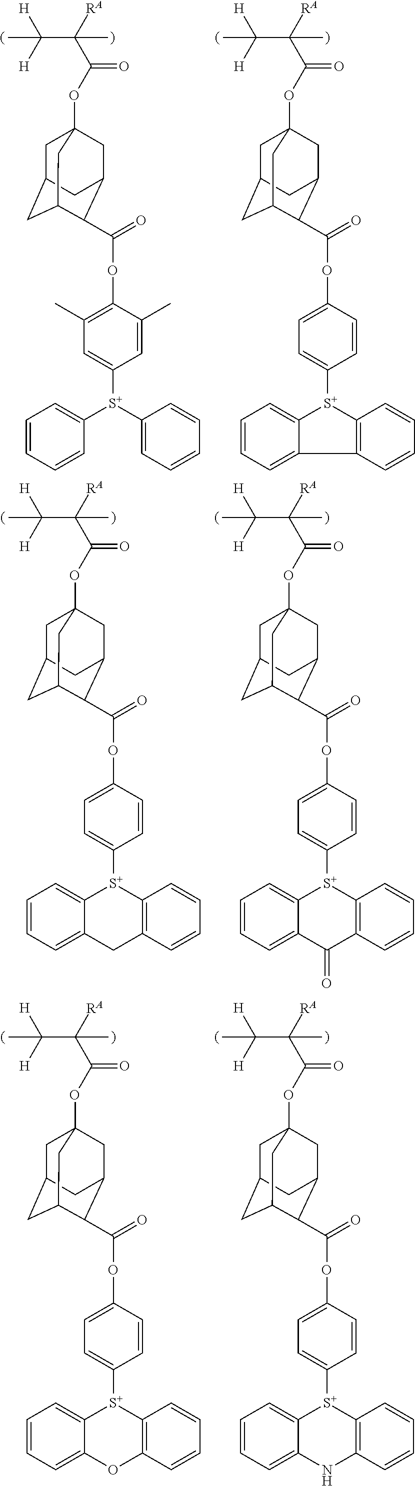

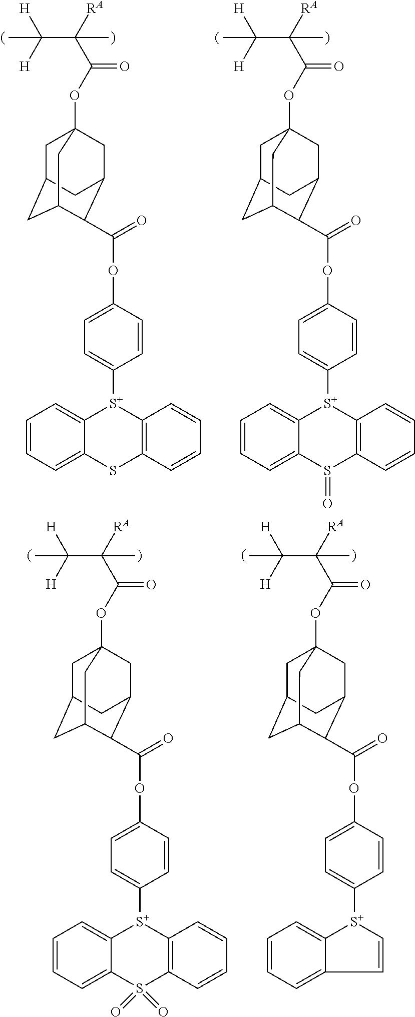

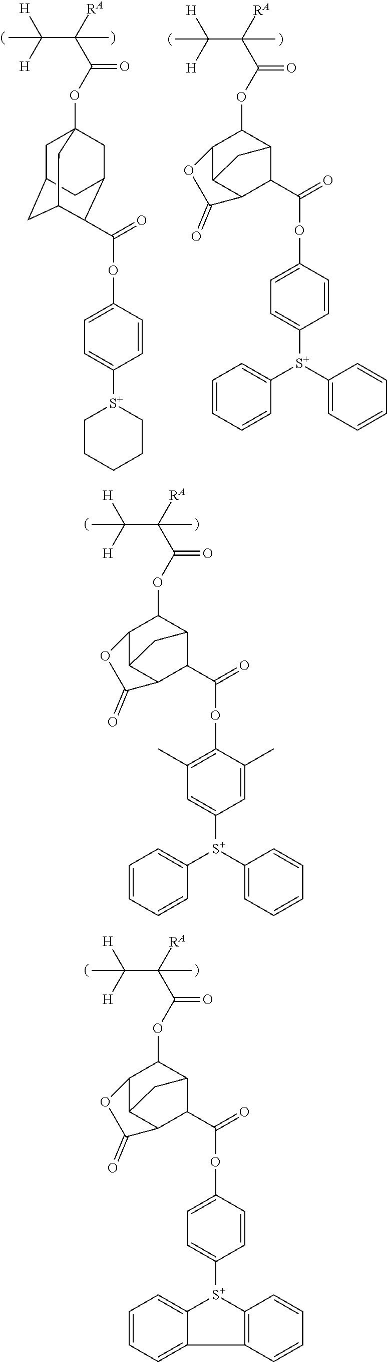

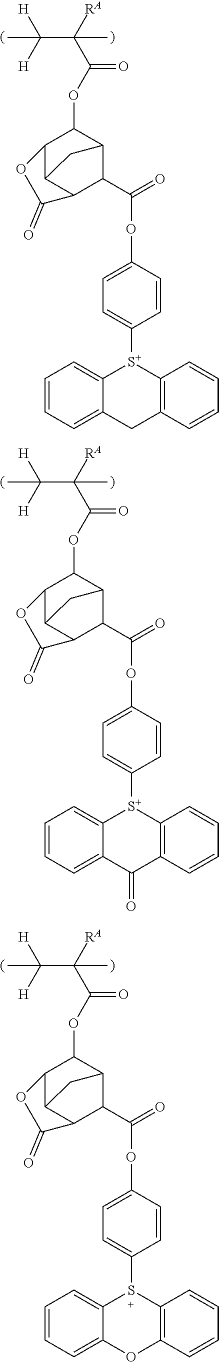

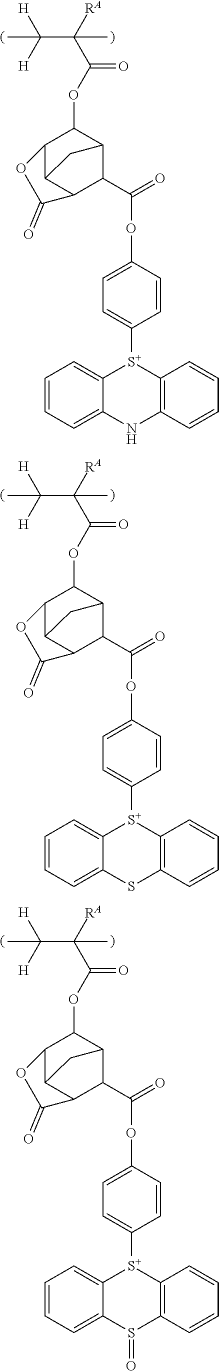

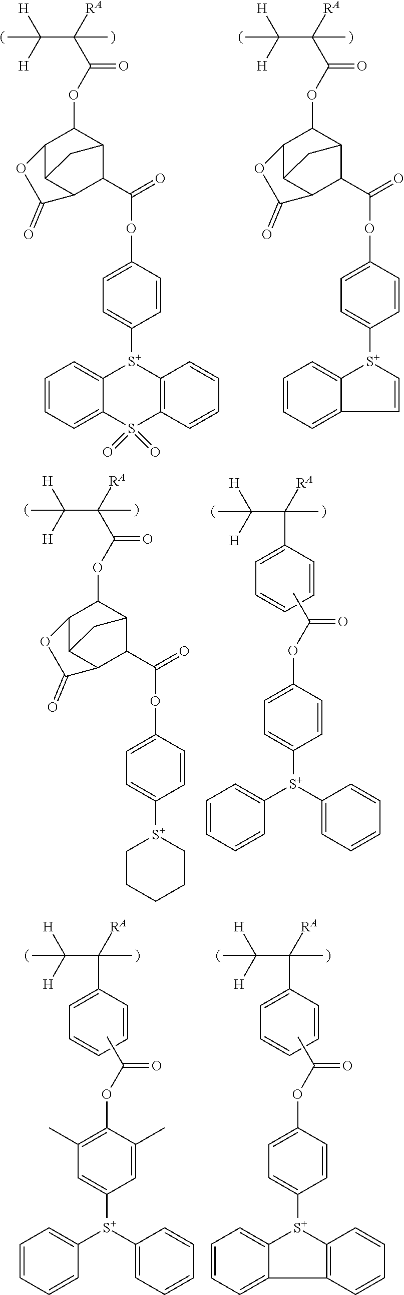

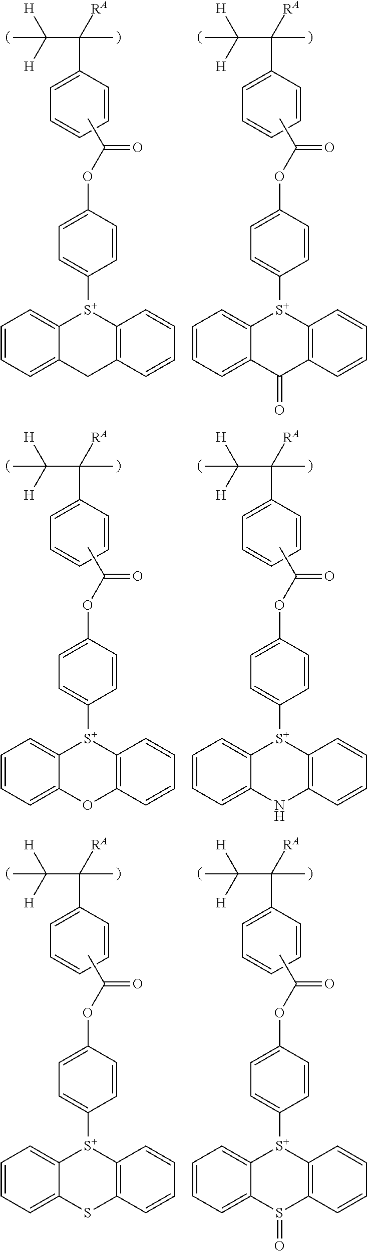

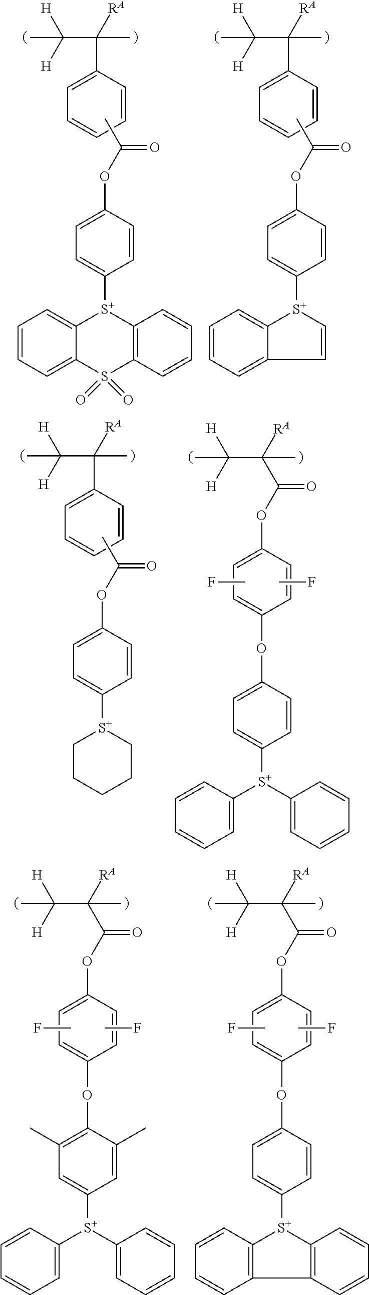

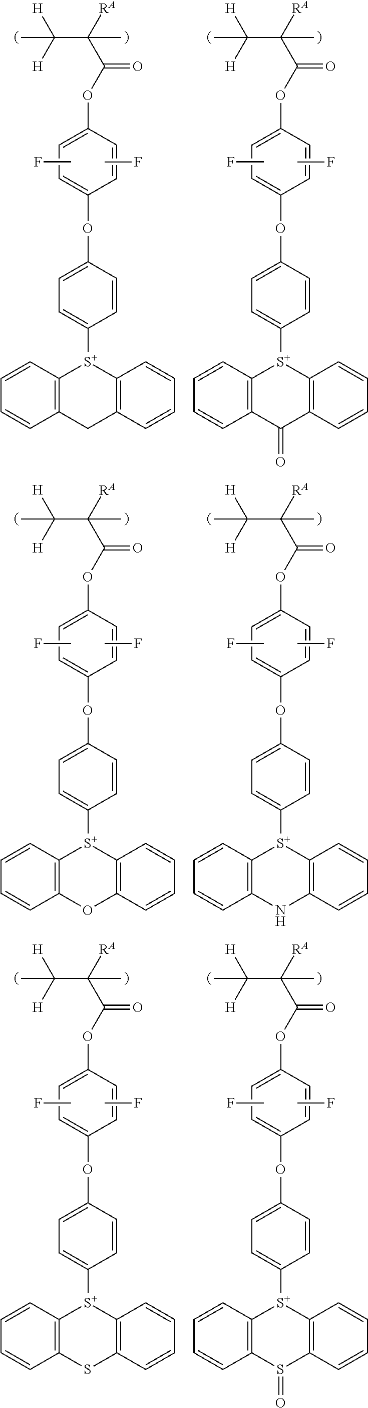

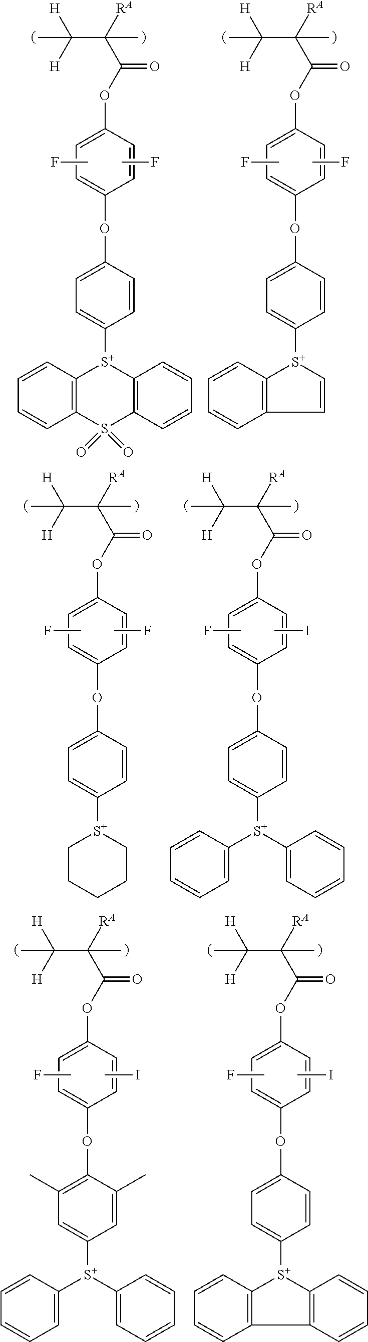

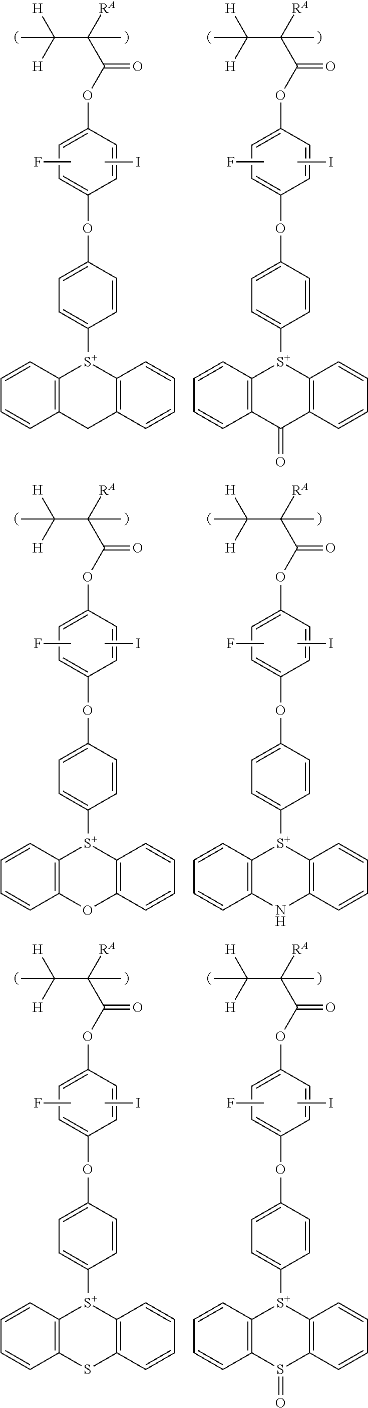

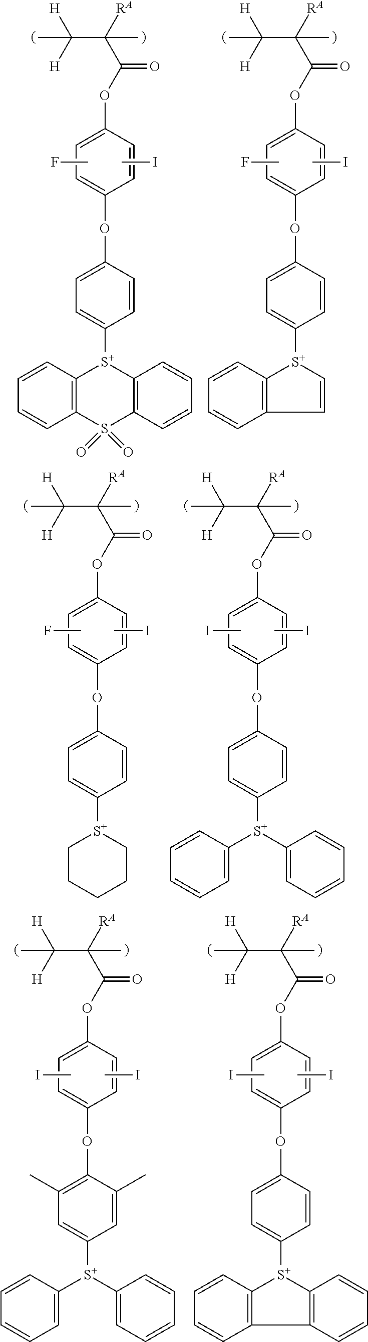

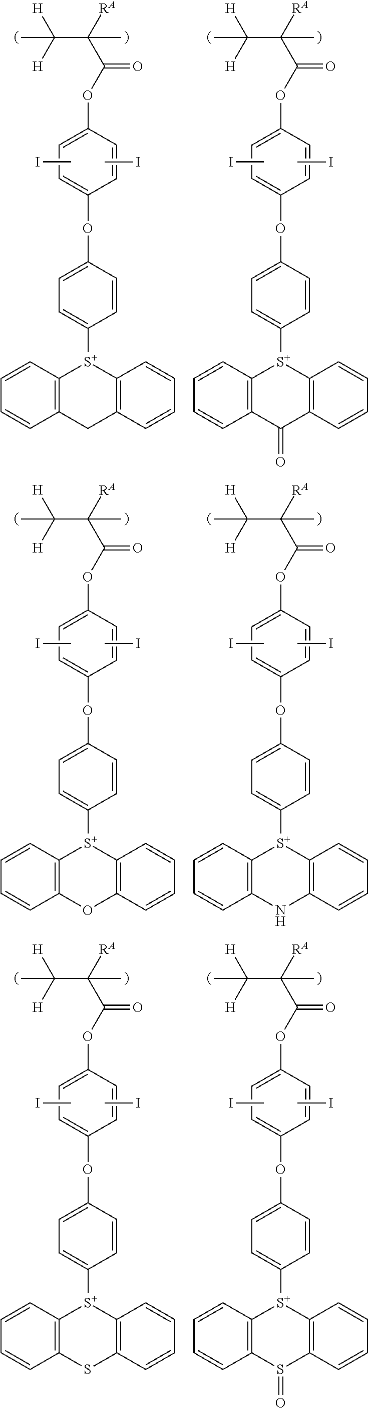

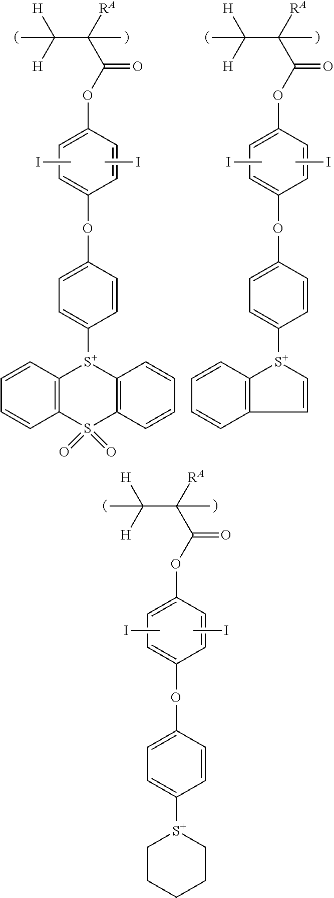

In the formula (c1), M⁻ is a non-nucleophilic counter ion, and The non-nucleophilic counter ion is preferably a halide ion, a sulfonate anion, an imide anion, or a methide anion. Specific examples of the halide ion include a chloride ion and a bromide ion. Specific examples of the sulfonate anion (sulfonate ion) include fluoroalkyl sulfonate ions such as a triflate ion, a 1,1,1-trifluoroethane sulfonate ion, and a nonafluorobutane sulfonate ion; aryl sulfonate ions such as a tosylate ion, a benzene sulfonate ion, a 4-fluorobenzenesulfonate ion, a 1,2,3,4,5-pentafluorobenzenesulfonate ion; and alkyl sulfonate ions such as a mesylate ion and a butanesulfonate ion. Specific examples of the imide anion (imide ion) include a bis(trifluoromethylsulfonyl)imide ion, a bis(perfluoroethylsulfonyl)imide ion, and a bis(perfluorobutylsulfonyl)imide ion. Specific examples of the methide anion (methide ion) include a tris(trifluoromethylsulfonyl) methide ion and a tris(perfluoroethylsulfonyl) methide ion.

Other examples of the non-nucleophilic counter ion include anions having the formulae (c1-1) to (c1-4).

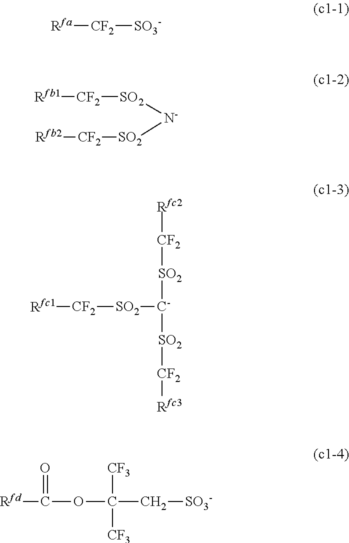

In the formula (c1-1), Rfa is a fluorine atom or a C₁-C₄₀ hydrocarbyl group which may contain a heteroatom, The hydrocarbyl group may be saturated or unsaturated and straight, branched, or cyclic. Specific examples thereof are as exemplified later for the hydrocarbyl group of R^{fa1} in the formula (c1-1-1).

As the anion having the formula (c1-1), an anion having the formula (c1-1-1) is preferred.

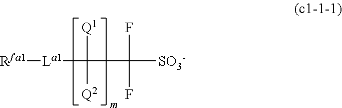

In the formula (c1-1-1), Q1 and Q2 are each independently a hydrogen atom, a fluorine atom, or a C₁-C₆ fluorinated saturated hydrocarbyl group, but at least one of Q1 and Q2 is preferably a trifluoromethyl group for improving the solvent solubility. m is 0, 1, 2, 3, or 4, and is particularly preferably 1. Ztal is a C₁-C₃₅ hydrocarbyl group which may contain a heteroatom, The heteroatom is preferably an oxygen atom, a nitrogen atom, a sulfur atom, a halogen atom, or the like, and more preferably an oxygen atom. As the hydrocarbyl group, a C₆-C₃₀ hydrocarbyl group is particularly preferred from the aspect of obtaining high resolution in fine pattern formation.

In the formula (c1-1-1), the C₁-C₃₅ hydrocarbyl group of R^{fa1} may be saturated or unsaturated and straight, branched, or cyclic. Specific examples thereof include C₁-C₃₅ alkyl groups such as a methyl group, an ethyl group, a n-propyl group, an isopropyl group, a n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; C₃-C₃₅ cyclic saturated hydrocarbyl groups such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; C₂-C₃₅ unsaturated aliphatic hydrocarbyl groups such as a 2-propenyl group and a 3-cyclohexenyl group; C₆-C₃₅ aryl groups such as a phenyl group, a 1-naphthyl group, a 2-naphthyl group, and a 9-fluorenyl group; C₇-C₃₅ aralkyl groups such as a benzyl group and a diphenylmethyl group; and a group obtained by combining these groups.

Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

In the formula (c1-1-1), Lal is a single bond, an ether bond, an ester bond, a sulfonate ester bond, a carbonate bond, or a carbamate bond, but is preferably an ether bond or an ester bond, and more preferably an ester bond from the aspect of synthesis.

Specific examples of the anion having the formula (c1-1) are shown below, but not limited thereto. In the following formulae. Q′ is as defined above, and Ac is an acetyl group.

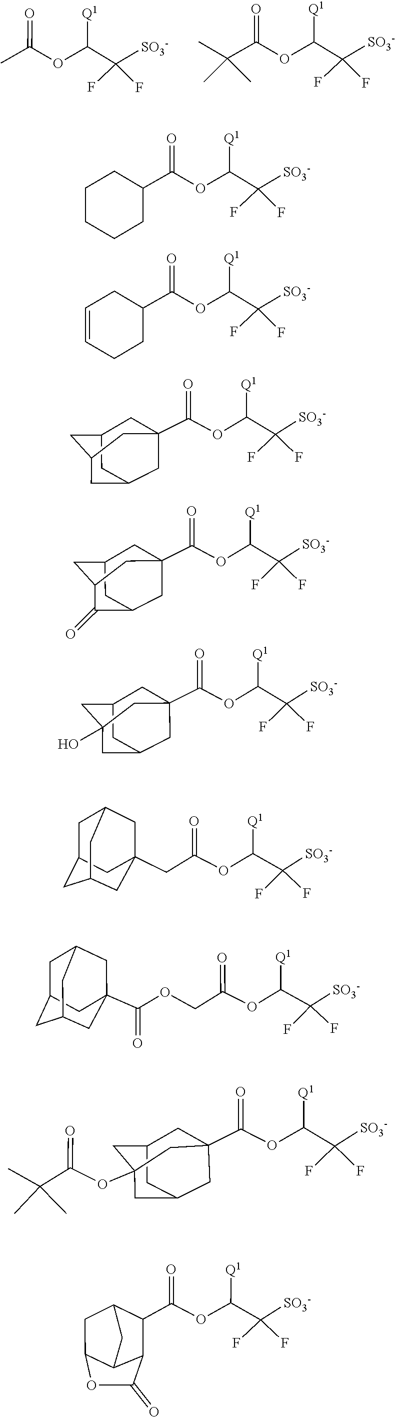

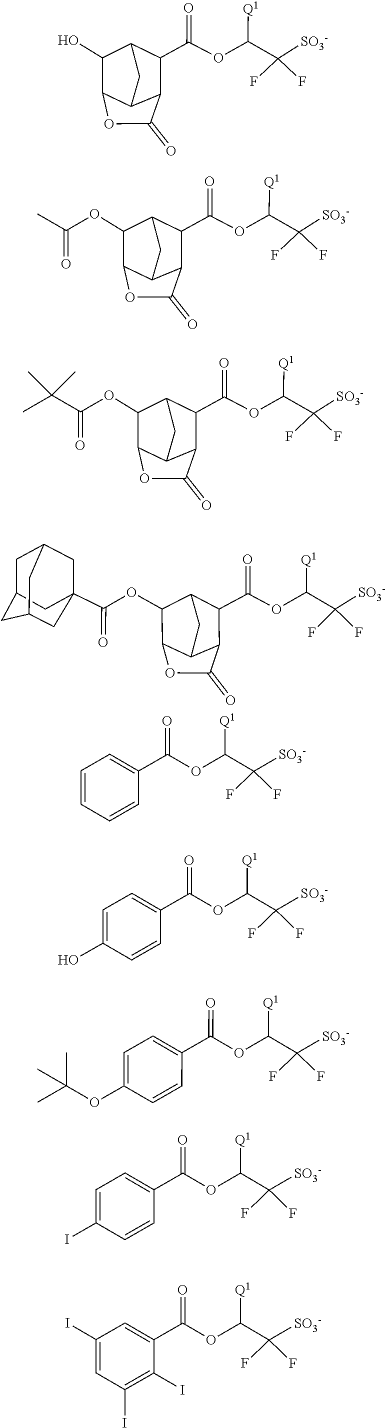

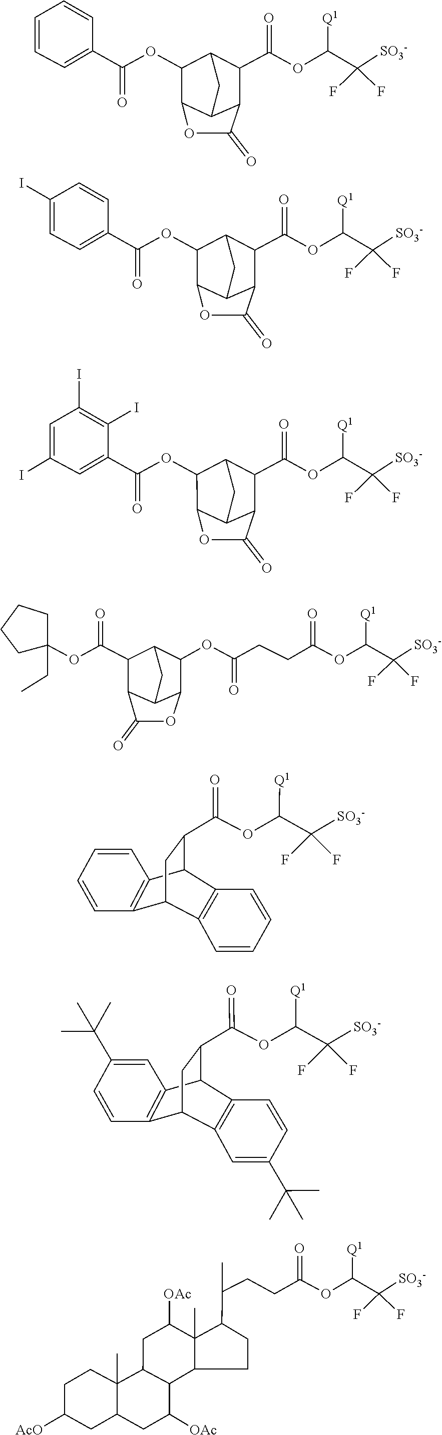

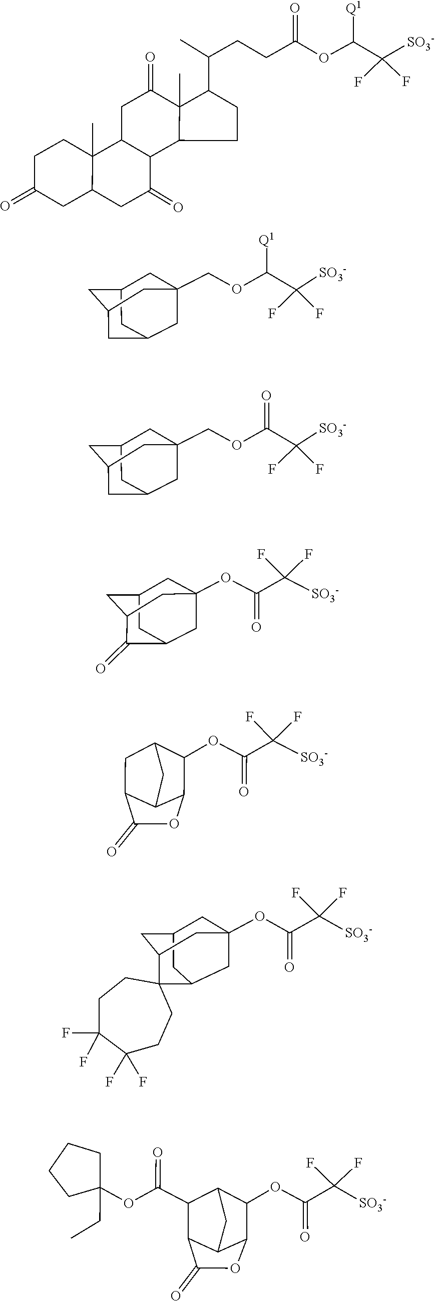

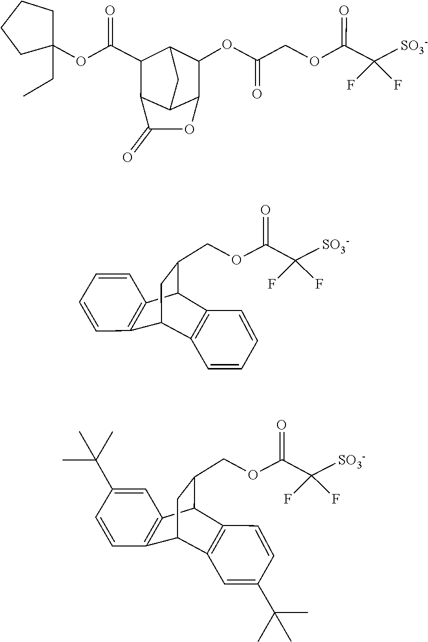

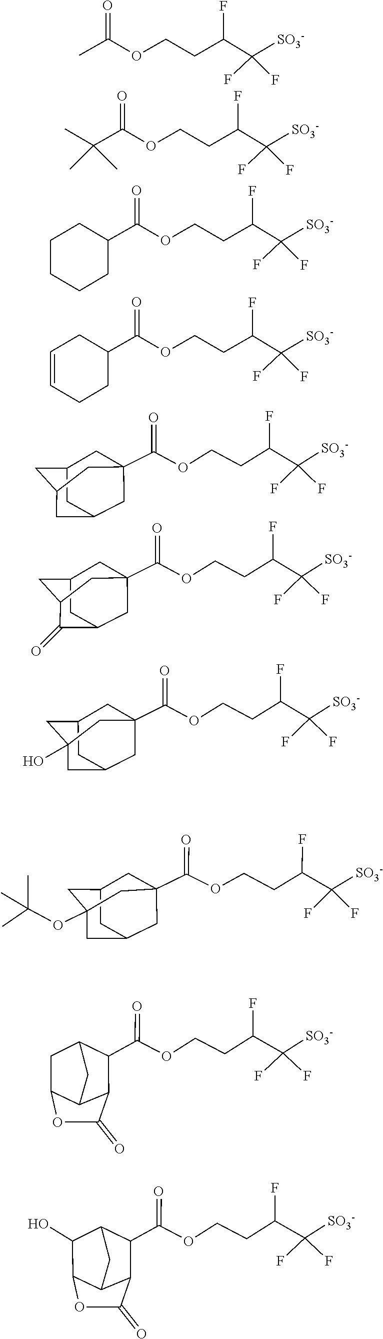

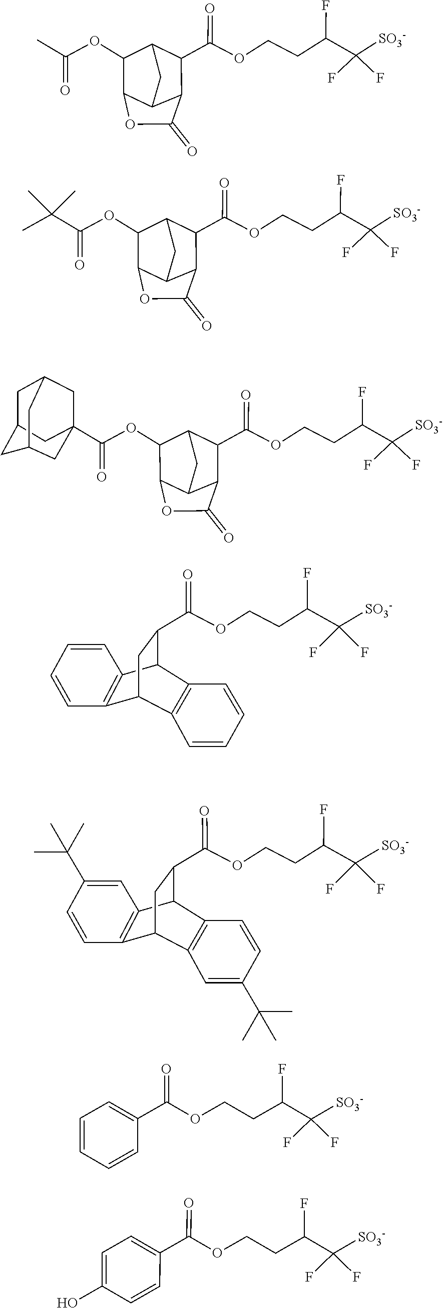

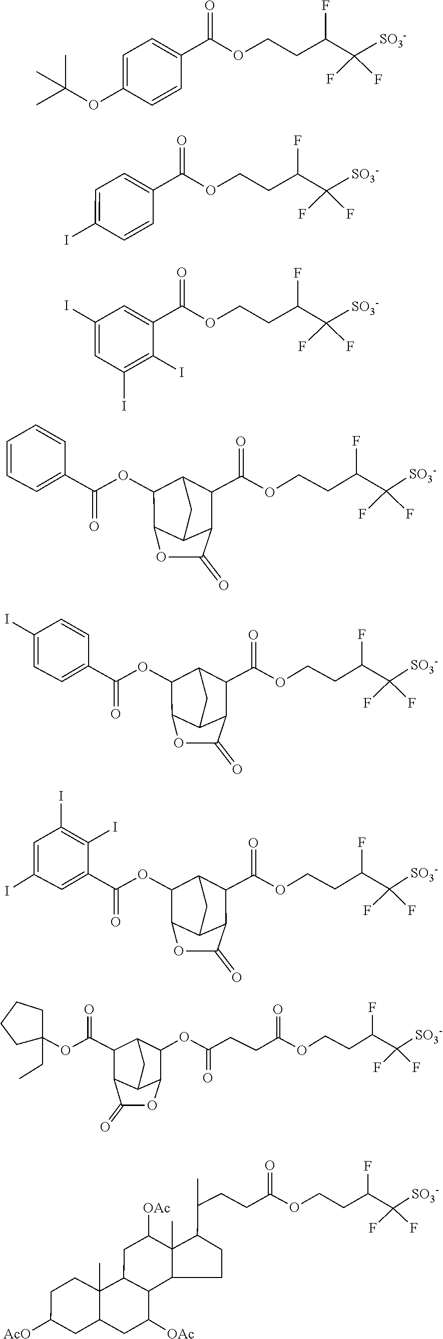

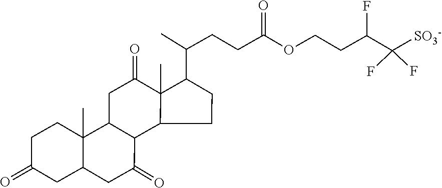

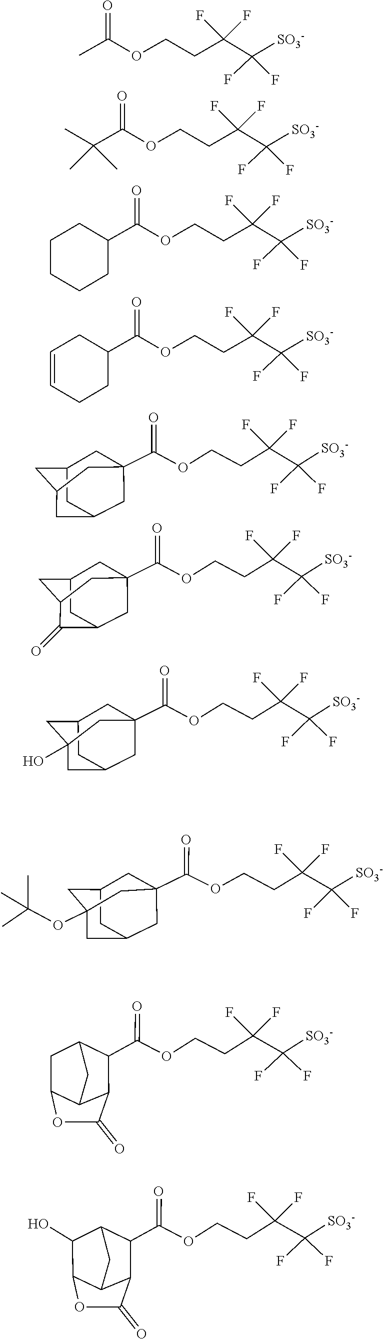

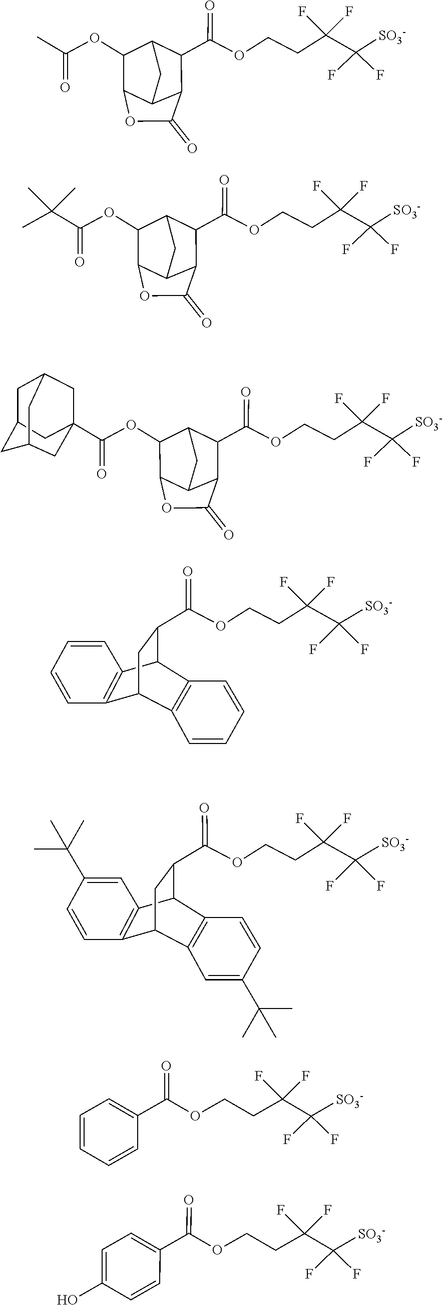

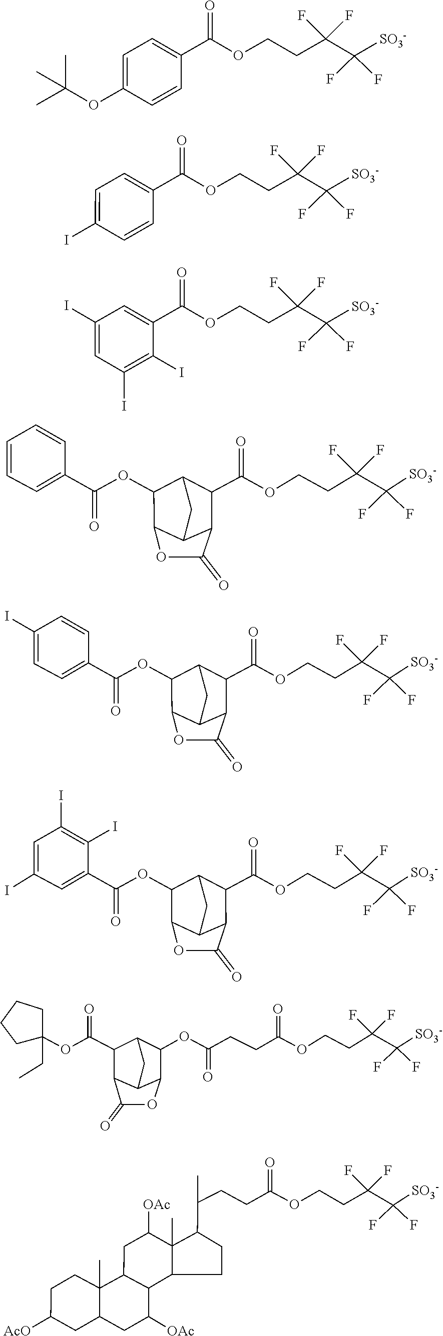

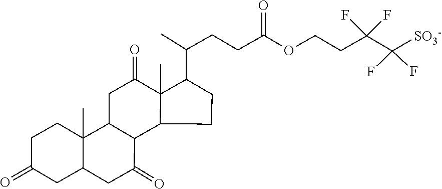

In the formula (c1-2), Rfb¹ and Rfb² are each independently a fluorine atom or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group of R^{fa1} in the formula (c1-1-1). Rfb¹ and Rfb² are preferably a fluorine atom or a straight C₁-C₄ fluorinated alkyl group. Rfb¹ and Rfb² may bond together to form a ring with a group (—CF₂—SO₂—N—SO₂—CF₂—) to which they are attached, and in this case, the group obtained by bonding Rfb¹ and Rfb² is preferably a fluorinated ethylene group or a fluorinated propylene group.

In the formula (c1-3), Rfc1, Rfc2, and Rfc3 are each independently a fluorine atom or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group of R^{fa1} in the formula (c1-1-1). Rfc¹, Rfc², and Rfc³ are preferably a fluorine atom or a straight C₁-C₄ fluorinated alkyl group. Rfc¹ and Rfc² may bond together to form a ring with a group (—CF₂—SO₂—C—SO₂—CF₂—) to which they are attached, and in this case, the group obtained by bonding Rfel and Rfc² is preferably a fluorinated ethylene group or a fluorinated propylene group.

In the formula (c1-4), Rfd is a C₁-C₄₀ hydrocarbyl group which may contain a heteroatom, The hydrocarbyl group may be saturated or unsaturated and straight, branched, or cyclic. Specific examples thereof are as exemplified above for the hydrocarbyl group of R^{fa1} in the formula (c1-1-1).

Specific examples of the anion having the formula (c1-4) are shown below, but not limited thereto.

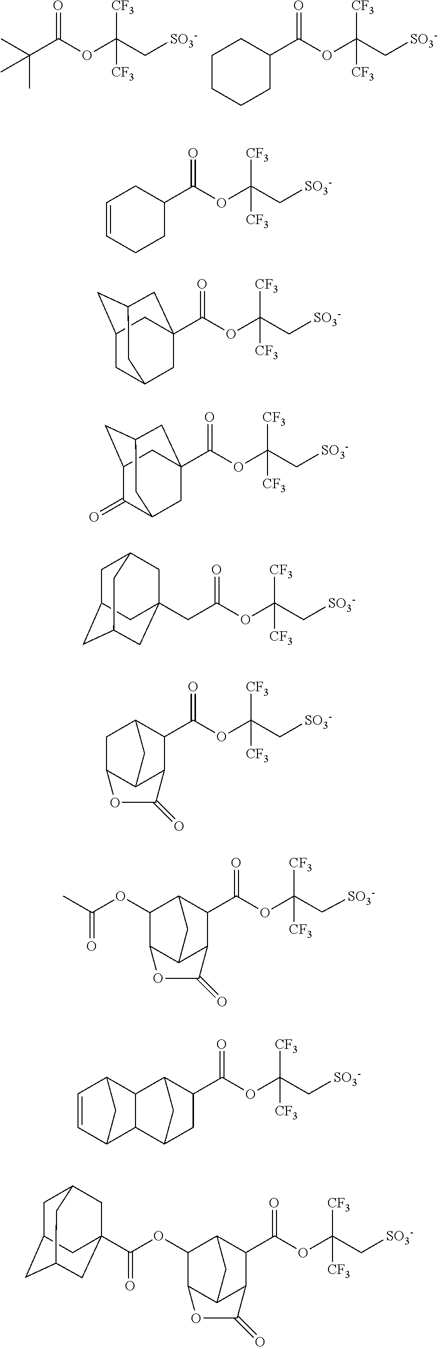

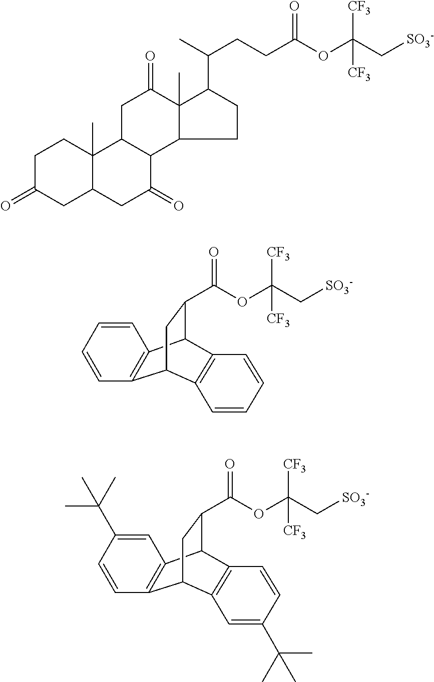

Examples of the non-nucleophilic counter ion further include an anion having an aromatic ring substituted with an iodine atom or a bromine atom. Specific examples of such an anion include anions having the formula (c1-5).

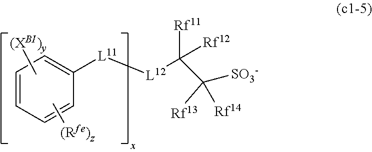

In the formula (c1-5), x is 1, 2, or 3, y is 1, 2, 3, 4, or 5, z is 0, 1, 2, or 3, meeting 1≤y+z≤5, y is preferably 1, 2, or 3, and more preferably 2 or 3, and z is preferably 0, 1, or 2.

In the formula (c1-5), XBI is an iodine atom or a bromine atom, and when x and/or y is 2 or more, XBI's may be identical or different.

In the formula (c1-5), L¹¹ is a single bond, an ether bond, an ester bond, or a C₁-C₆ saturated hydrocarbylene group which may contain an ether bond or an ester bond. The saturated hydrocarbylene group may be straight, branched, or cyclic.

In the formula (c1-5), L¹² is a single bond or a C₁-C₂₀ divalent linking group when x is 1, and a C₁-C₂₀ (x+1)-valent linking group when x is 2 or 3, and the linking group may contain an oxygen atom, a sulfur atom, or a nitrogen atom.

In the formula (c1-5), Rfe is a hydroxy group, a carboxy group, a fluorine atom, a chlorine atom, a bromine atom, or an amino group, or a C₁-C₂₀ hydrocarbyl group, a C₁-C₂₀ hydrocarbyloxy group, a C₂-C₂₀ hydrocarbylcarbonyl group, a C₂-C₂₀ hydrocarbyloxycarbonyl group, a C₂-C₂₀ hydrocarbylcarbonyloxy group, or a C₁-C₂₀ hydrocarbylsulfonyloxy group, which may contain a fluorine atom, a chlorine atom, a bromine atom, a hydroxy group, an amino group, or an ether bond, or —N(Rf^{feA})(R^{feB}), —N(R^{feC})—C(═O)—R^{feD}, or —N(R^{fec})—C(═O)—O—R^{feD}. R^{feA} and R^{feB} are each independently a hydrogen atom or a C₁-C₆ saturated hydrocarbyl group. R^{feC} is a hydrogen atom or a C₁-C₆ saturated hydrocarbyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-C₁₅ aralkyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched, or cyclic. The hydrocarbyl group, the hydrocarbyloxy group, the hydrocarbylcarbonyl group, the hydrocarbyloxycarbonyl group, the hydrocarbylcarbonyloxy group, and the hydrocarbylsulfonyloxy group may be straight, branched, or cyclic. When x and/or z is 2 or more, a plurality of Rfe's may be identical or different.

Among them, R^{fe} is preferably a hydroxy group, —N(R^{feC})—C(═O)—R^{feD}, —N(R^{fec})—C(═O)—O—R^{feD}, a fluorine atom, a chlorine atom, a bromine atom, a methyl group, a methoxy group, or the like.

In the formula (c1-5), Rf¹¹ to Rf¹⁴ are each independently a hydrogen atom, a fluorine atom, or a trifluoromethyl group, and at least one of them is a fluorine atom or a trifluoromethyl group. Rf¹¹ and Rf¹² may be combined to form a carbonyl group. In particular, both Rf¹³ and Rf¹⁴ are preferably fluorine atoms.

Specific examples of the anion having the formula (c1-5) are shown below, but not limited thereto. In the following formulae. X^{BI} is as defined above.

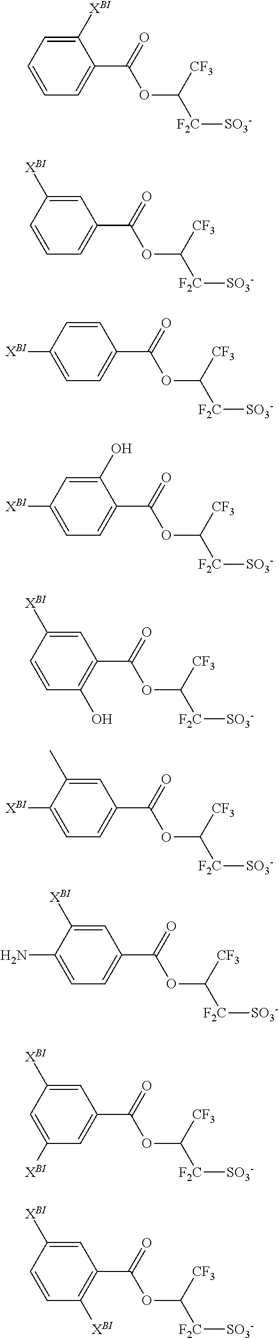

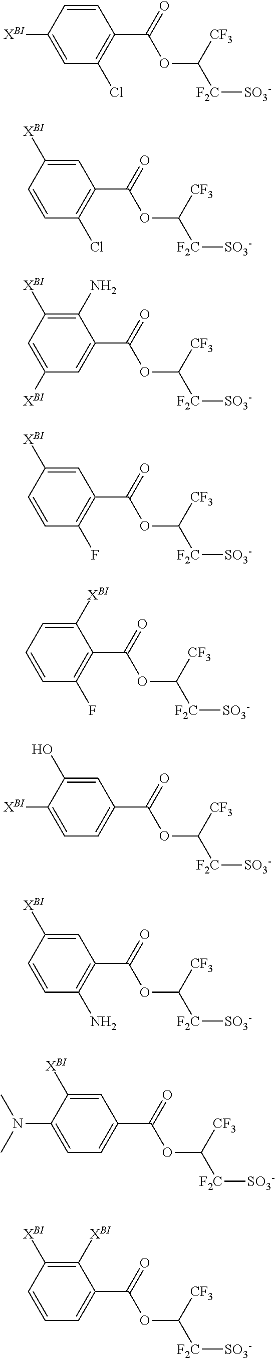

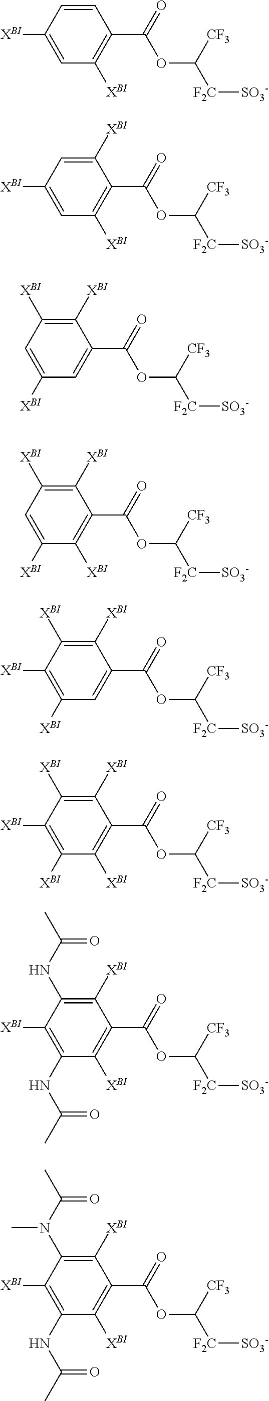

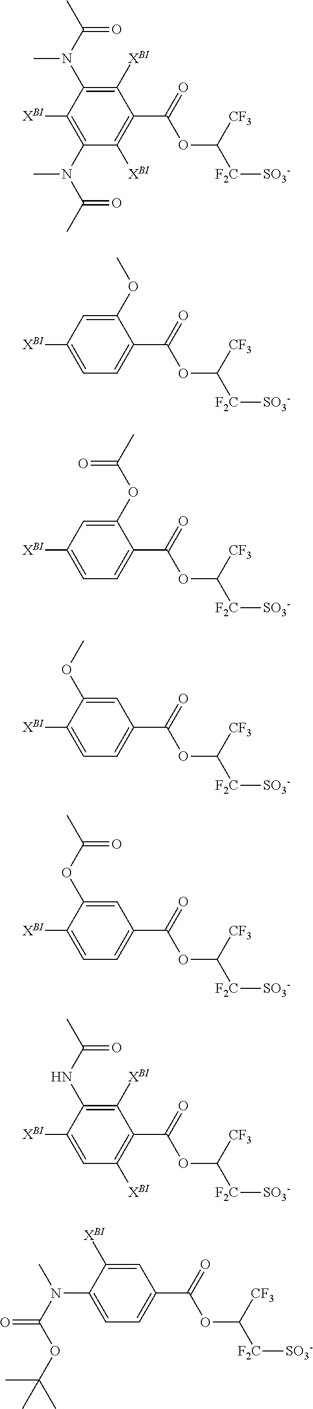

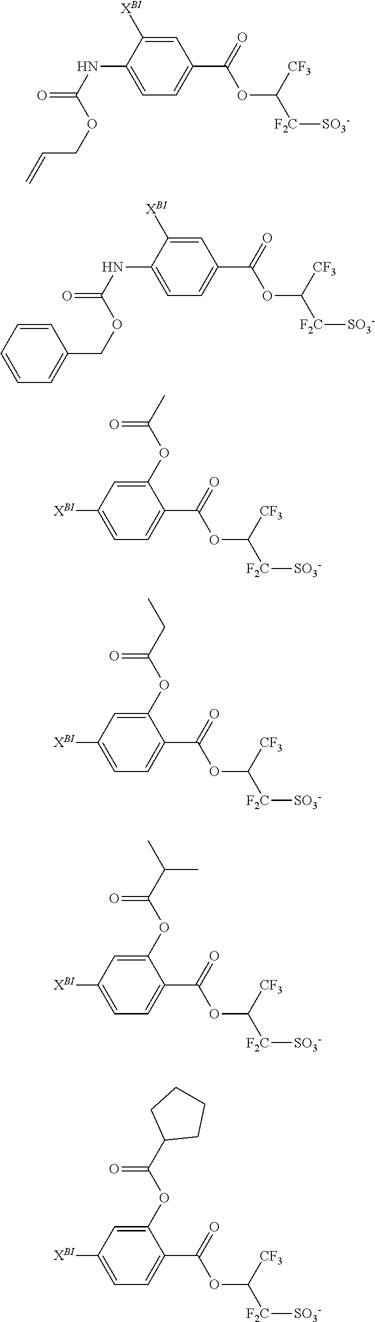

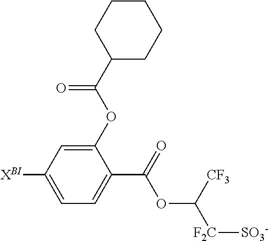

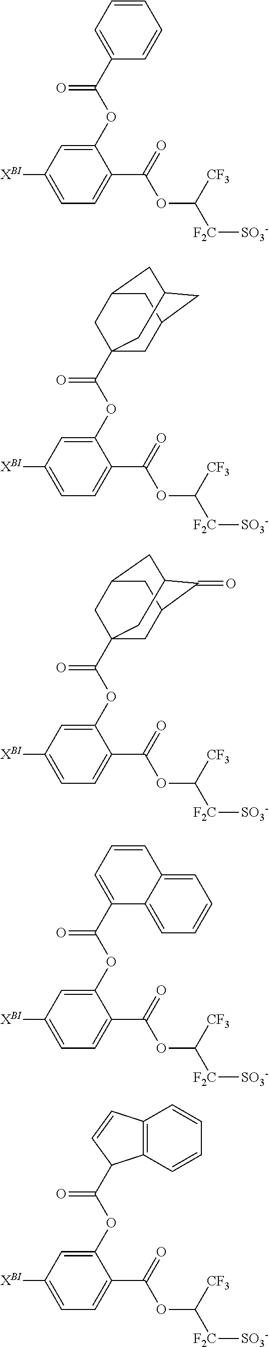

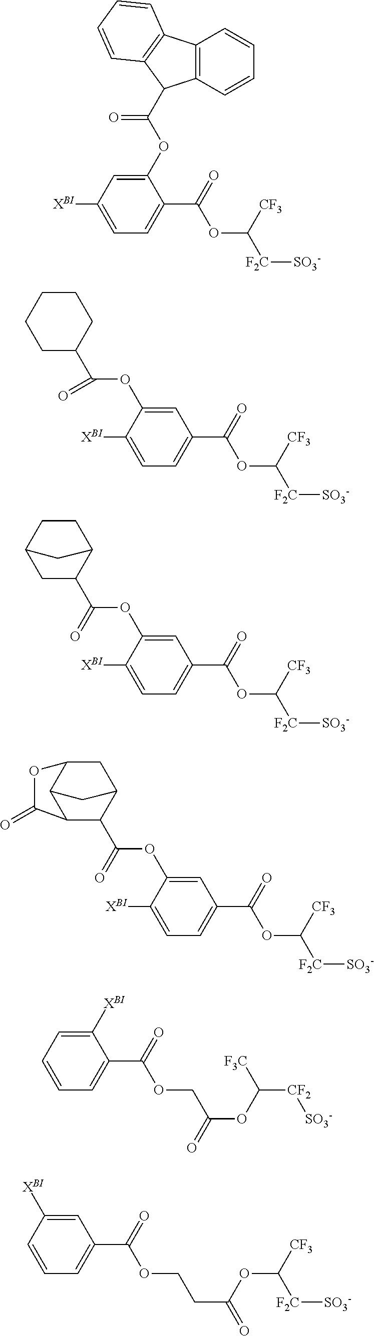

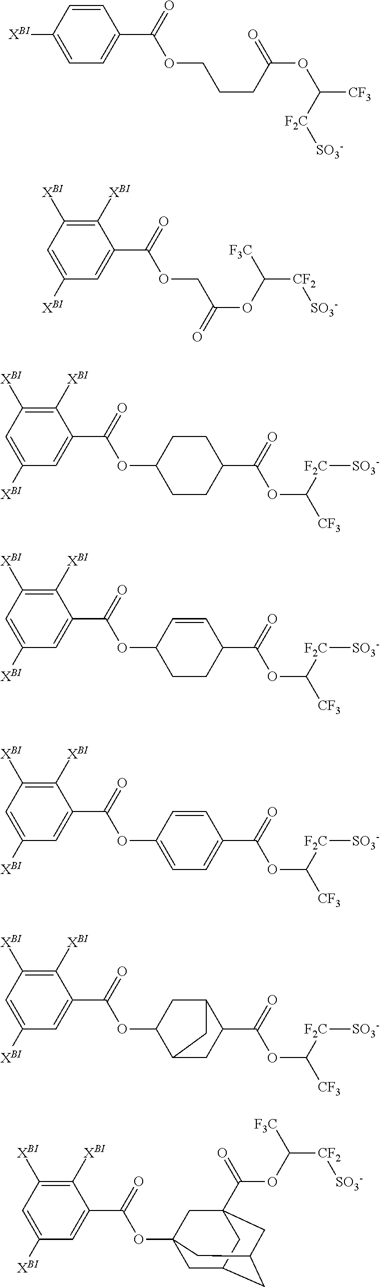

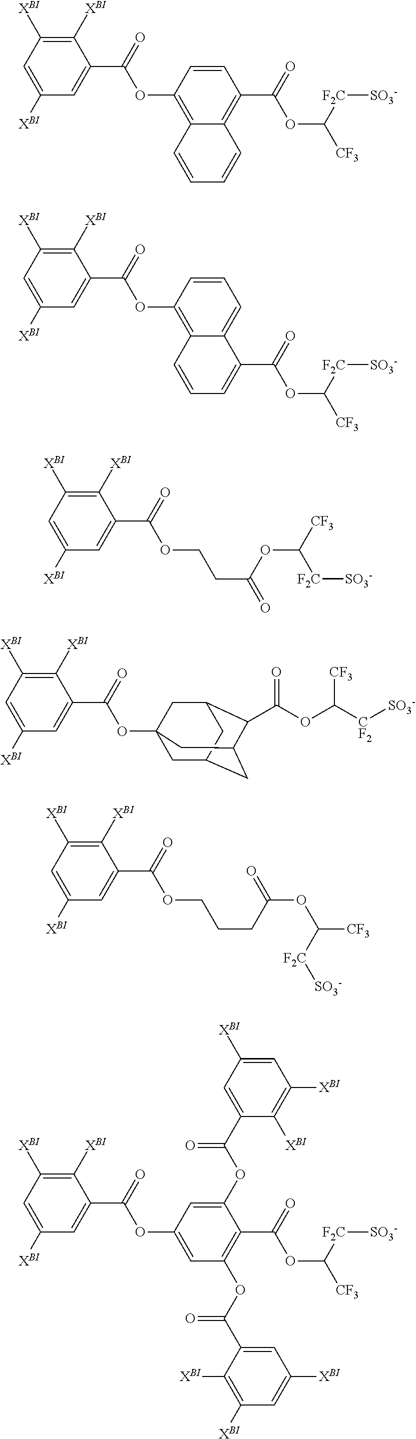

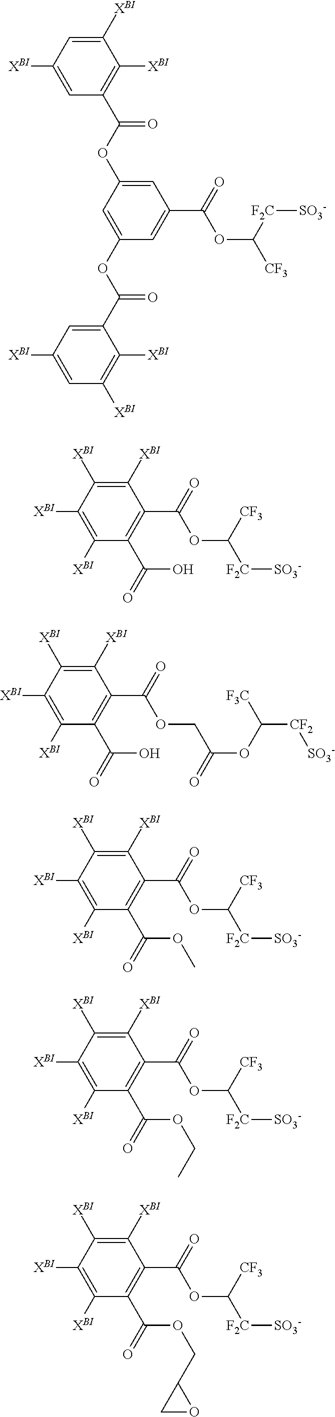

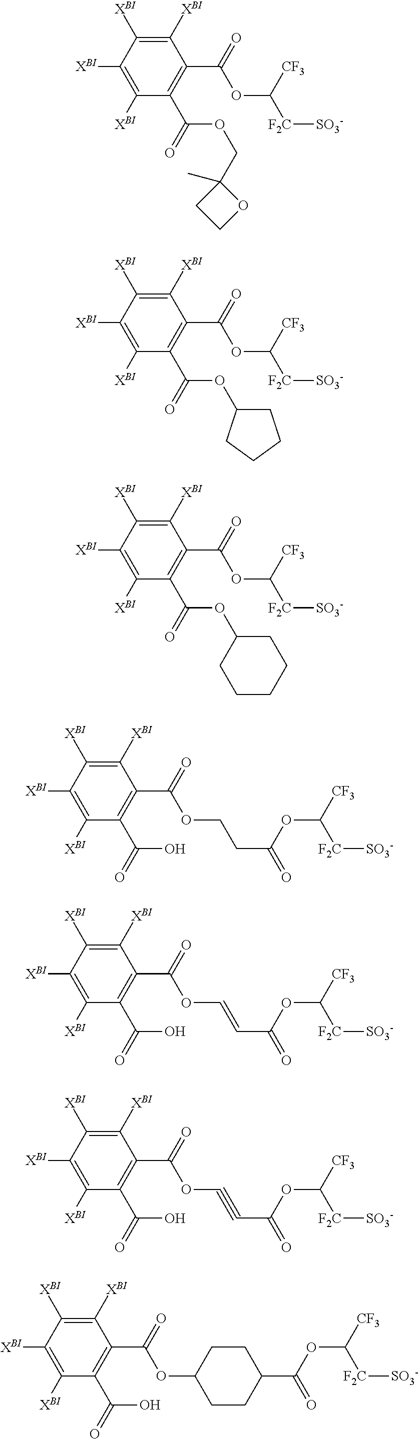

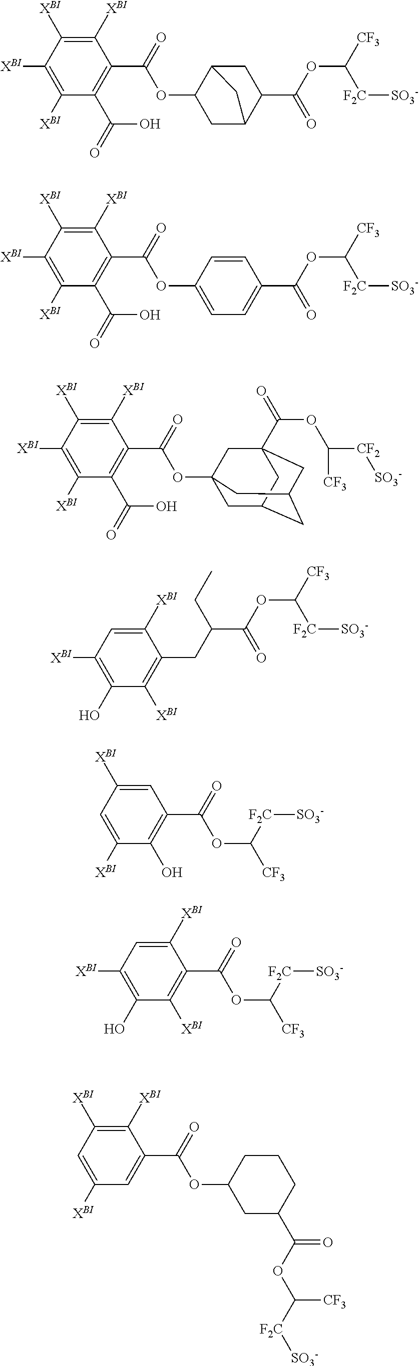

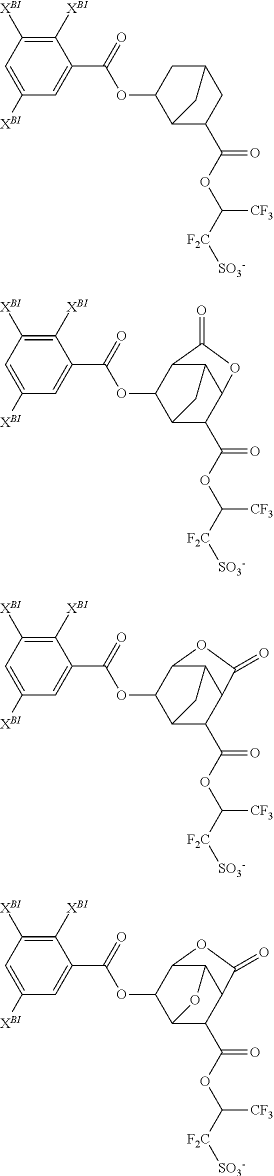

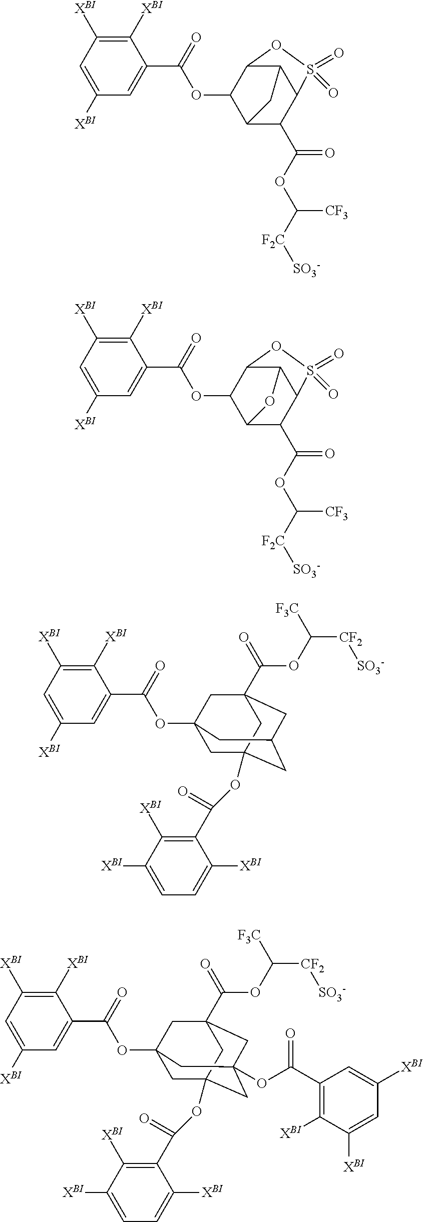

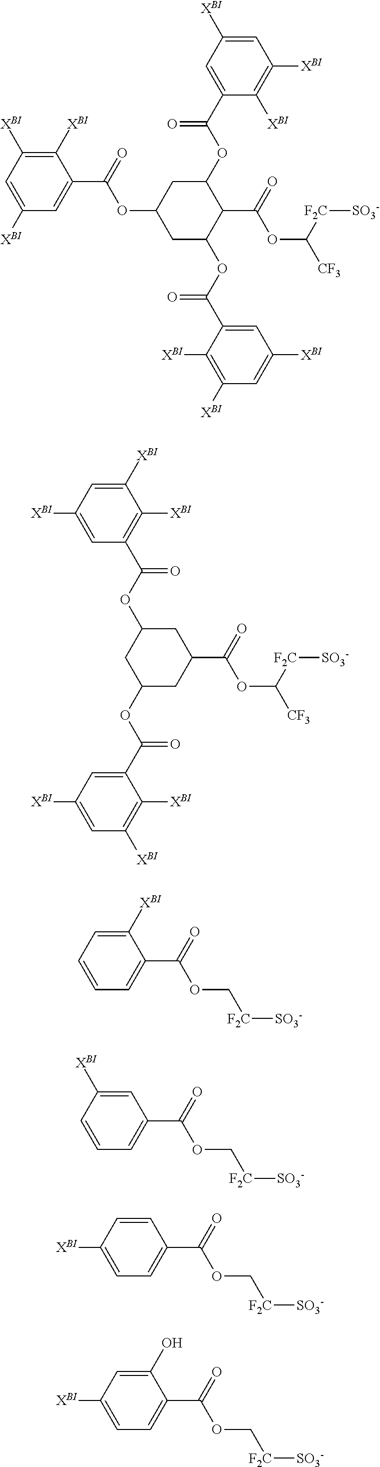

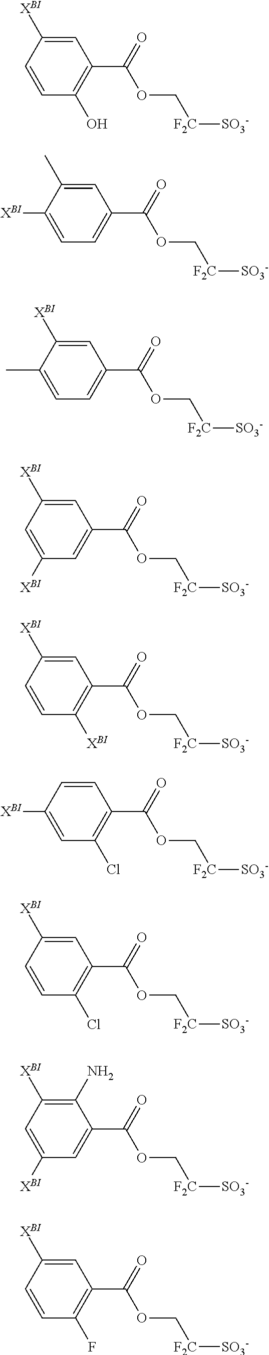

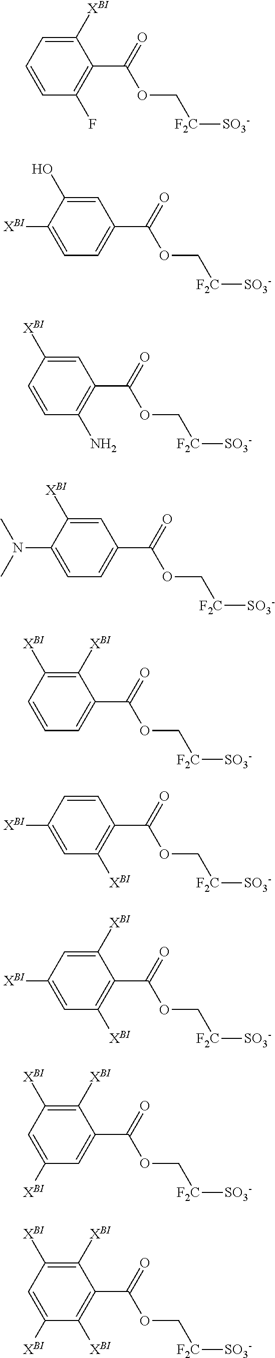

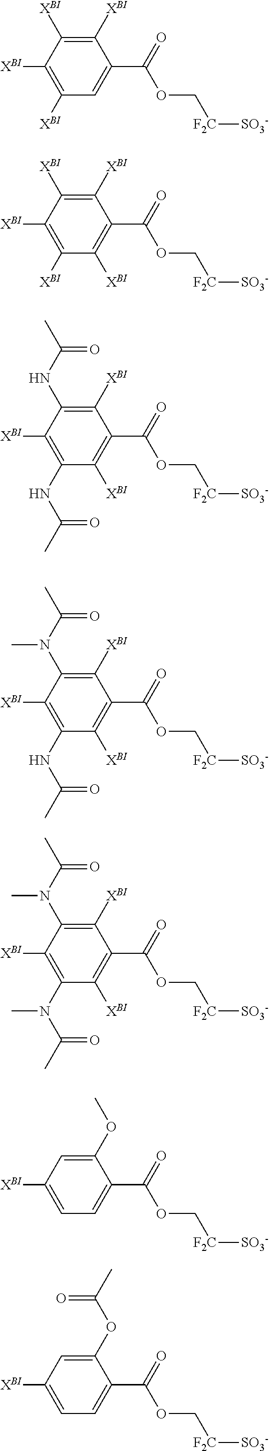

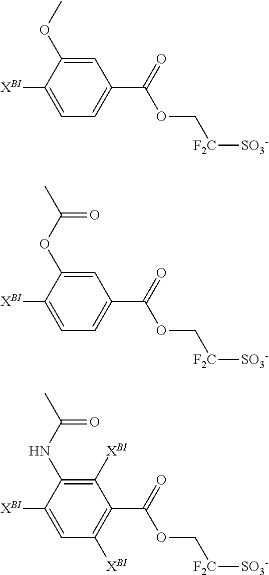

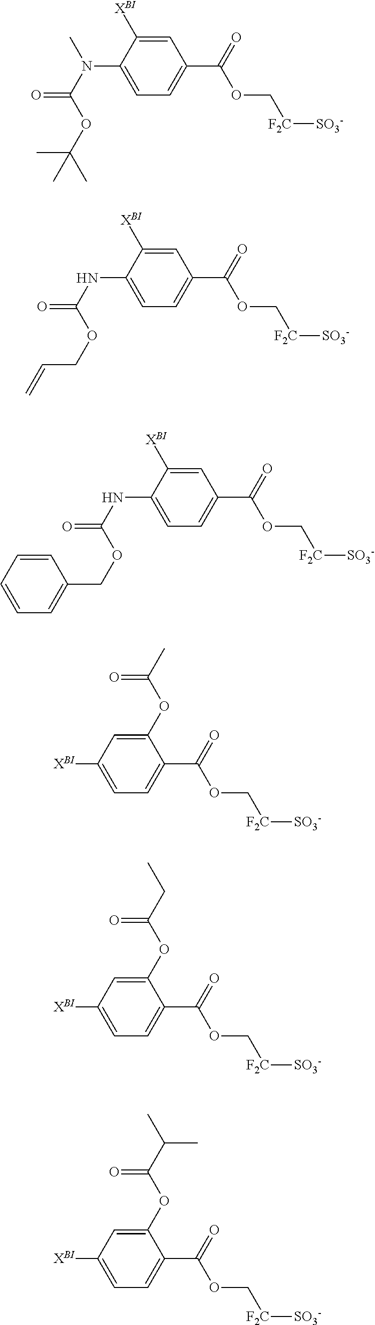

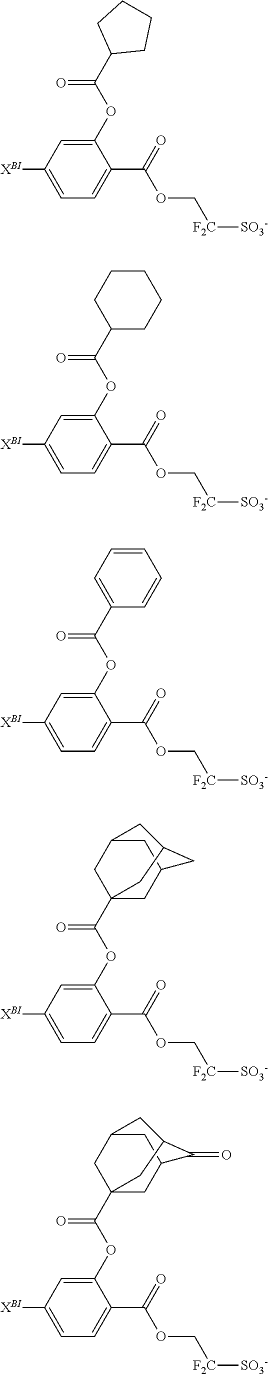

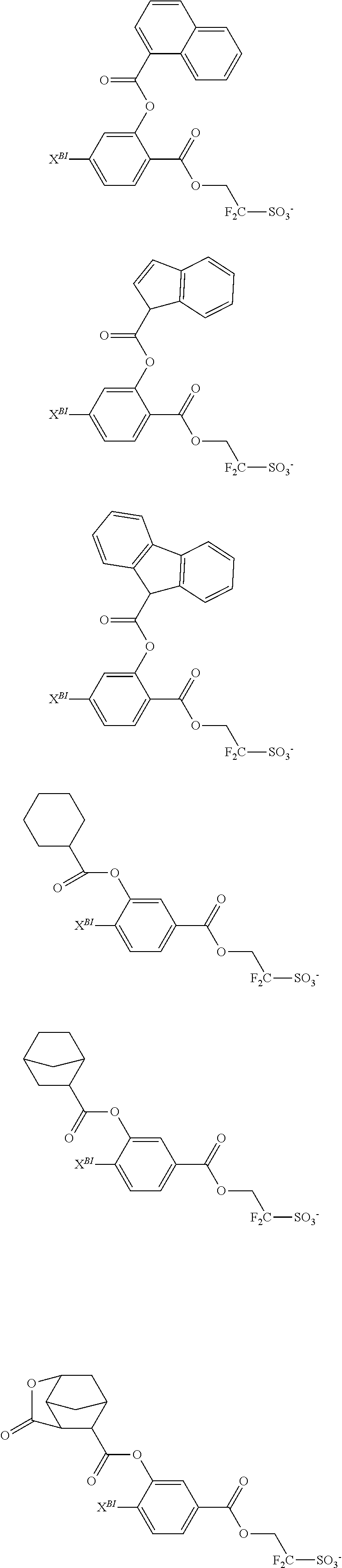

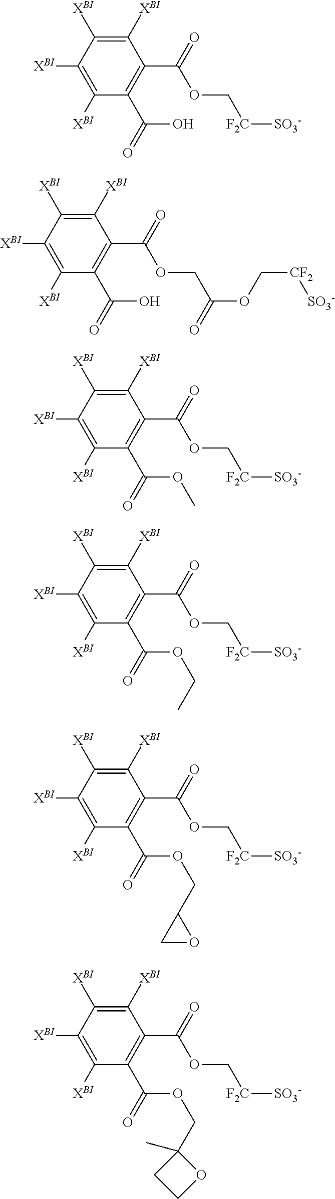

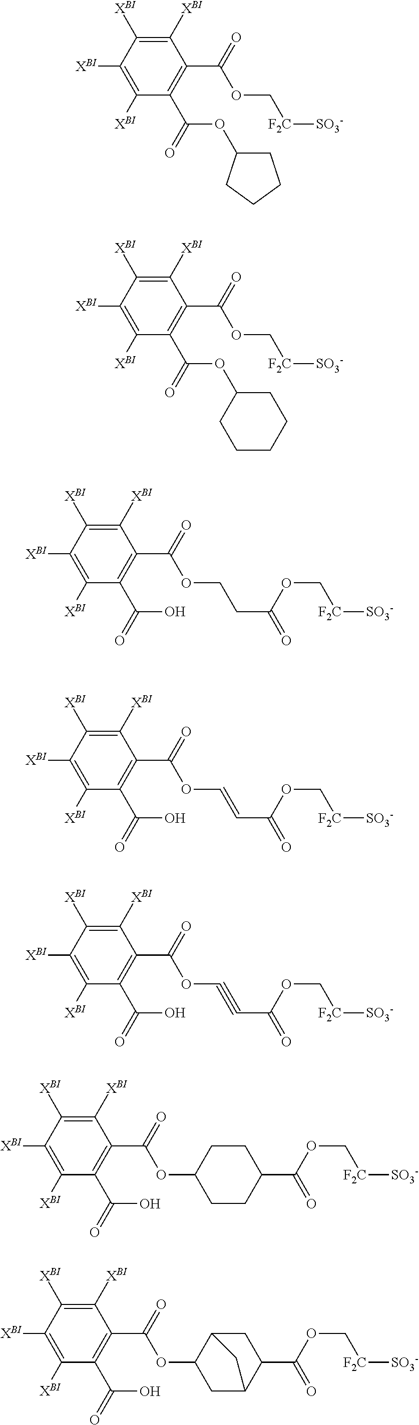

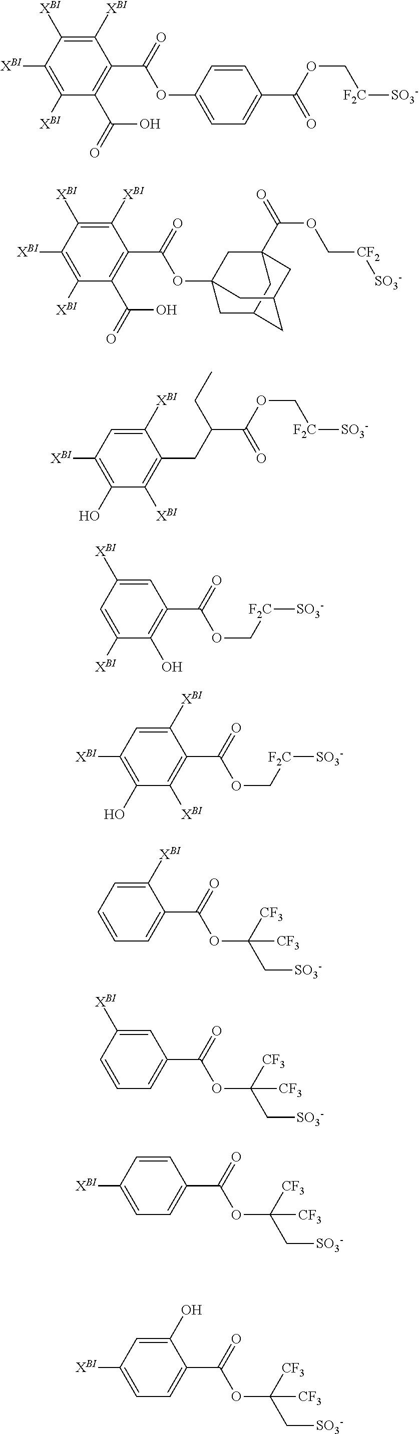

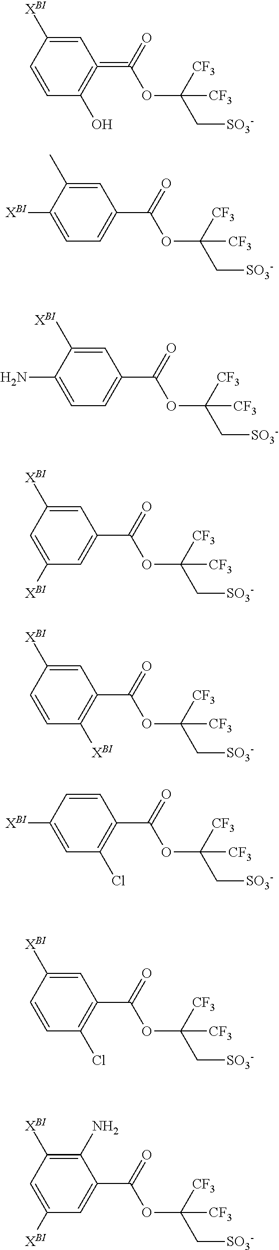

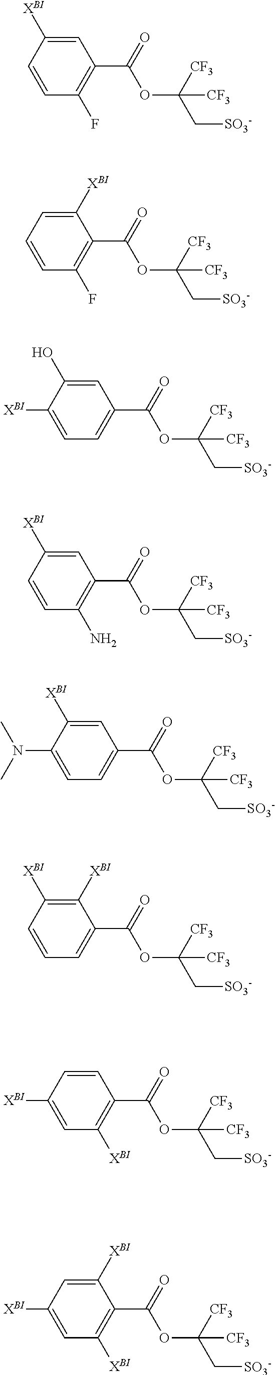

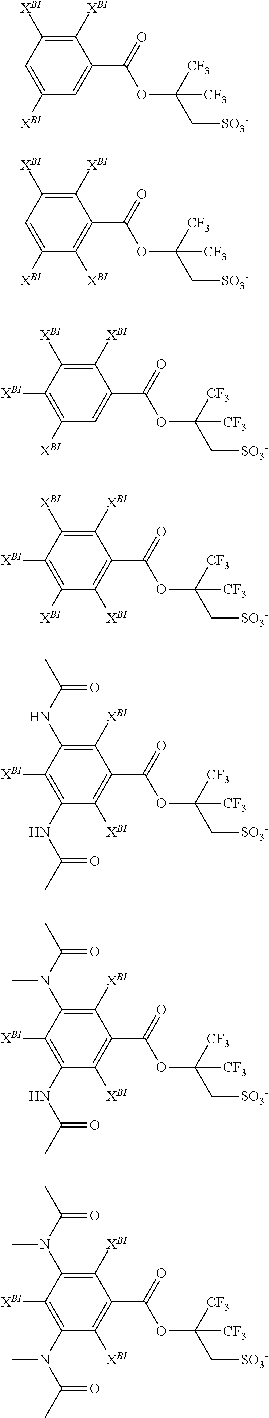

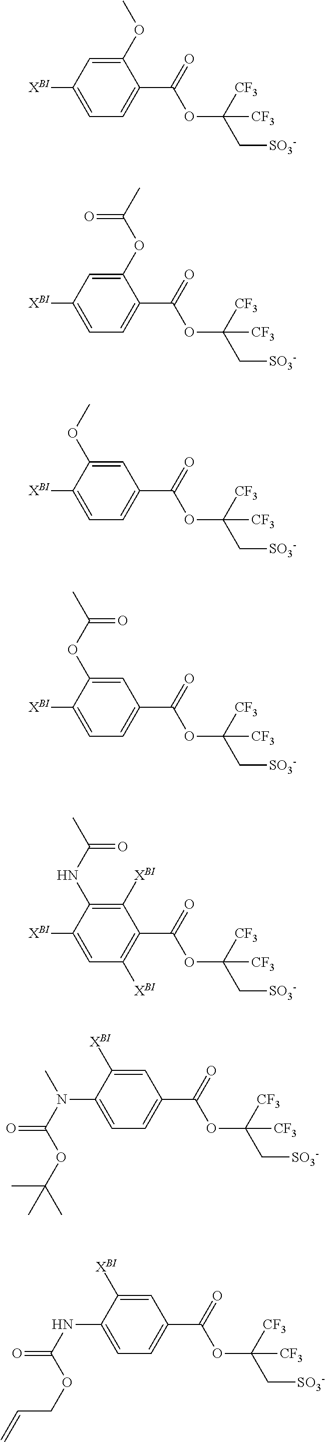

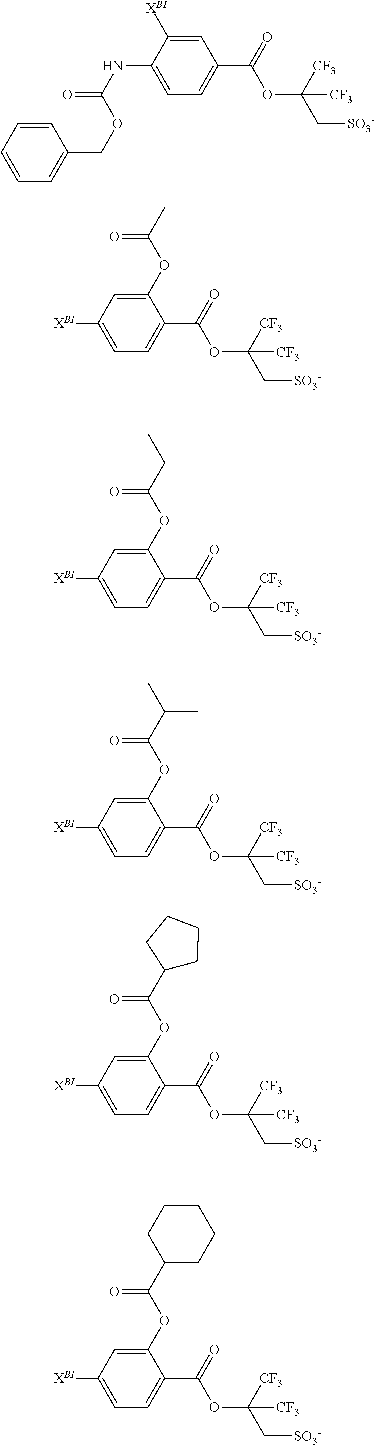

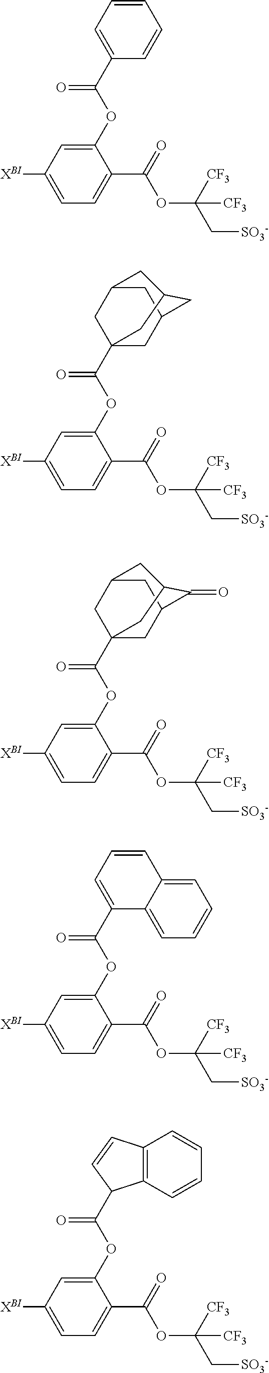

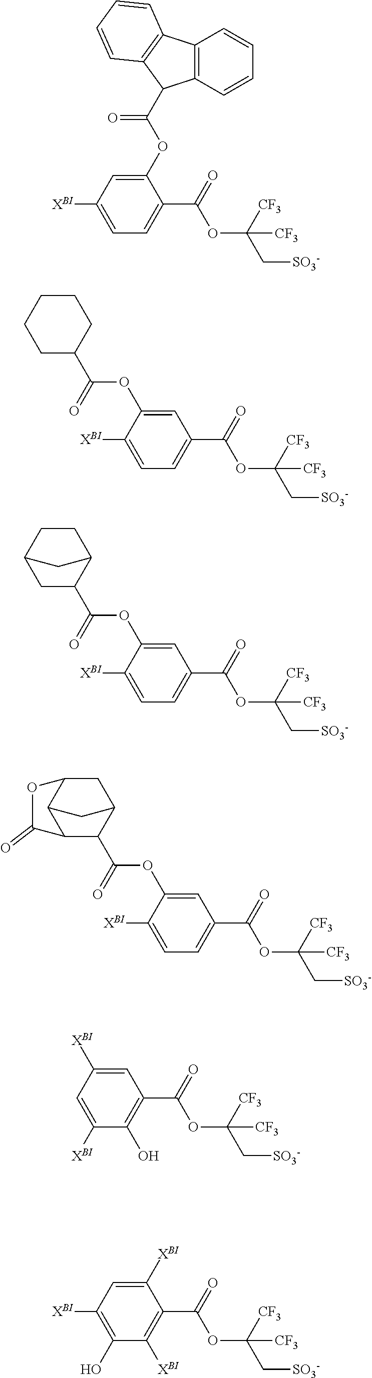

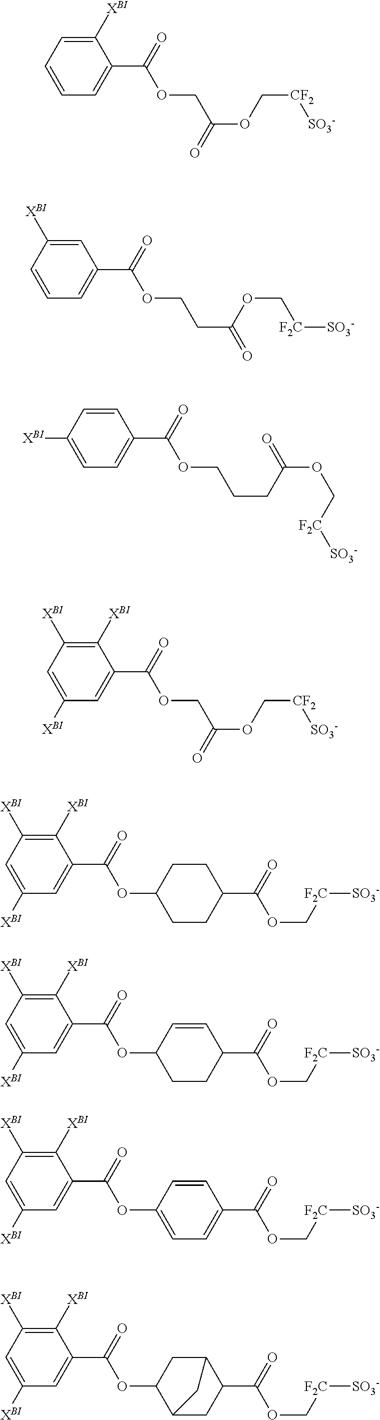

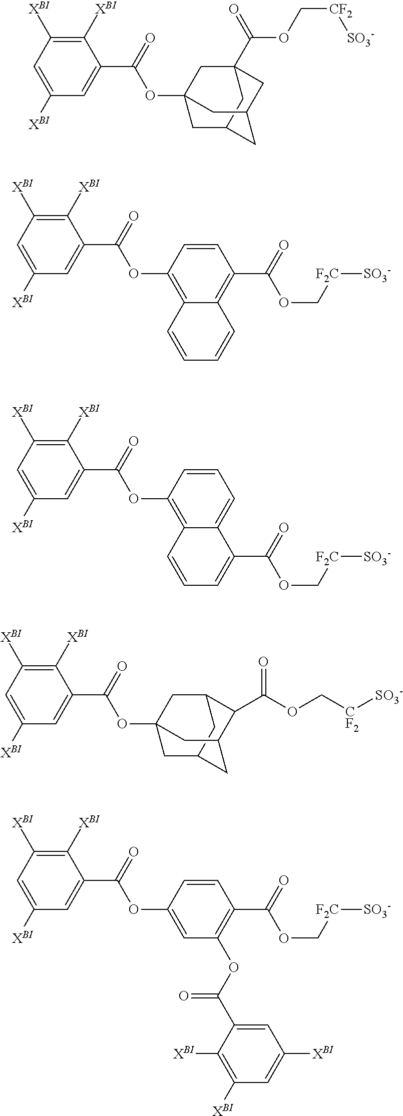

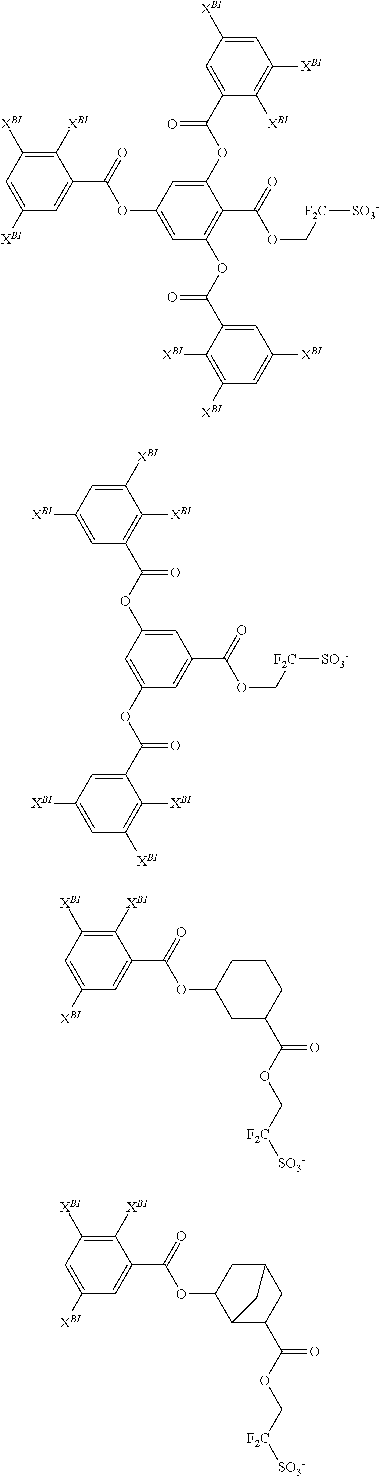

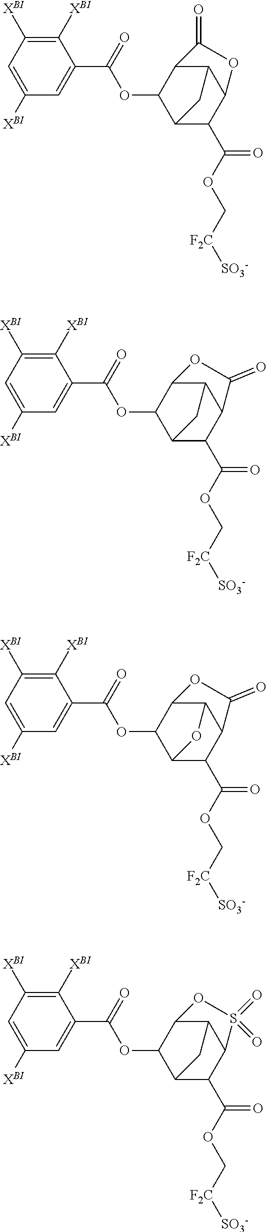

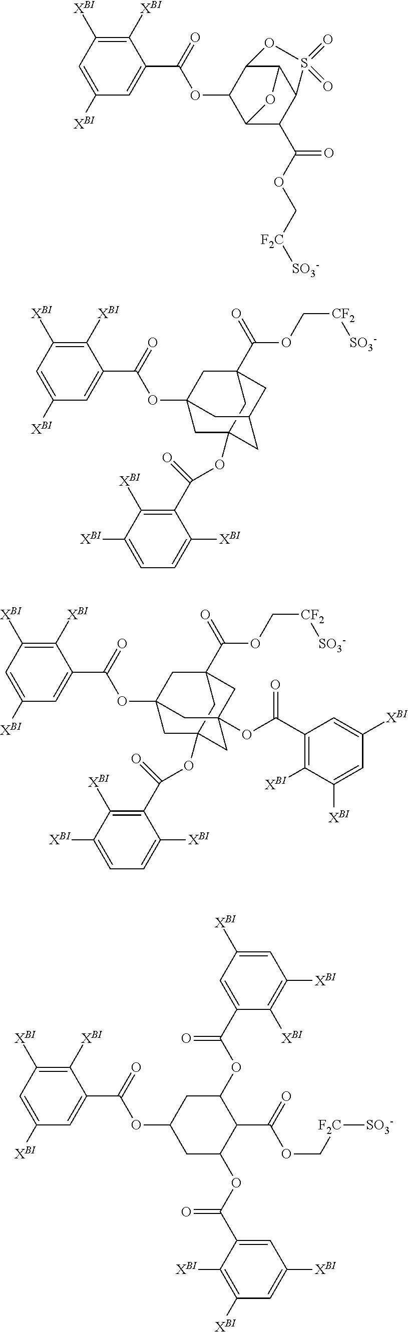

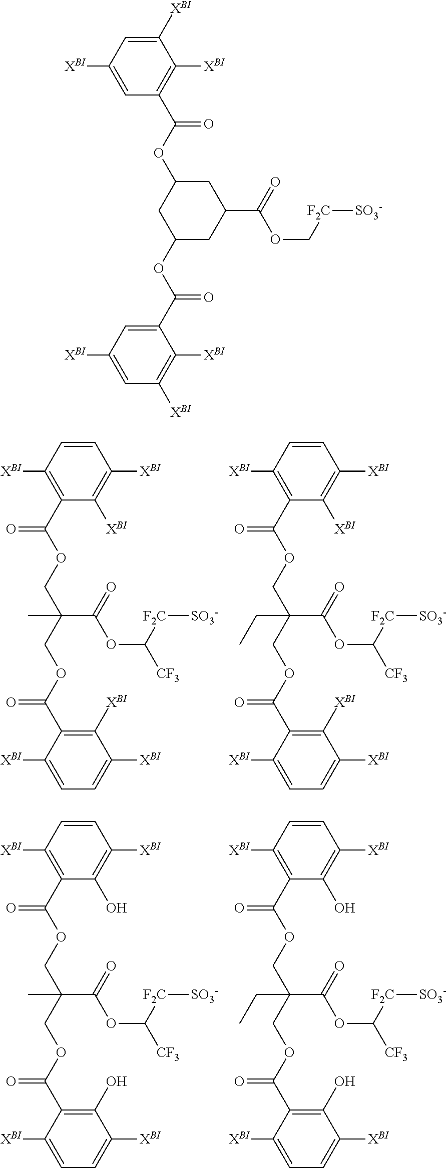

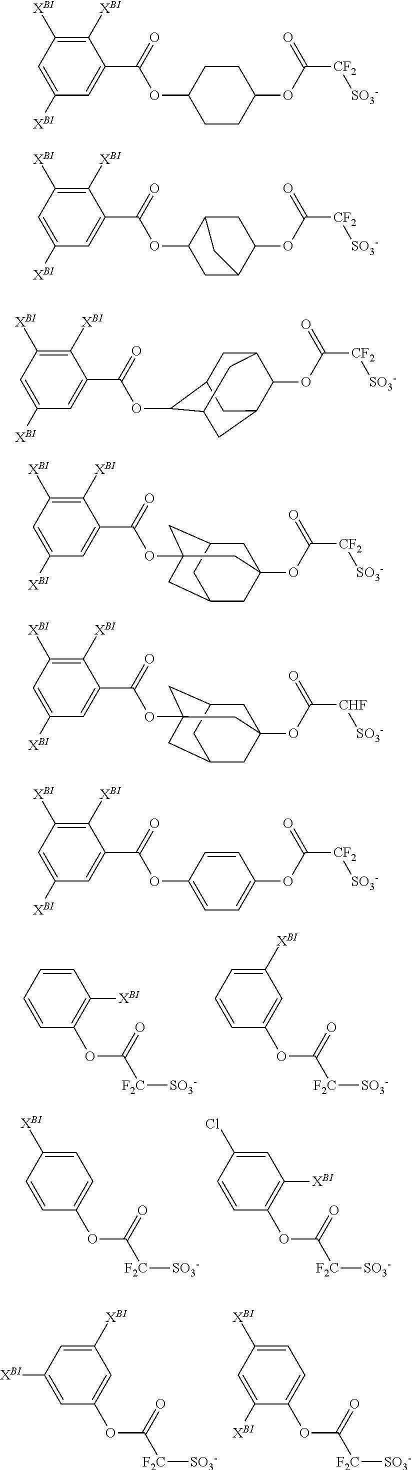

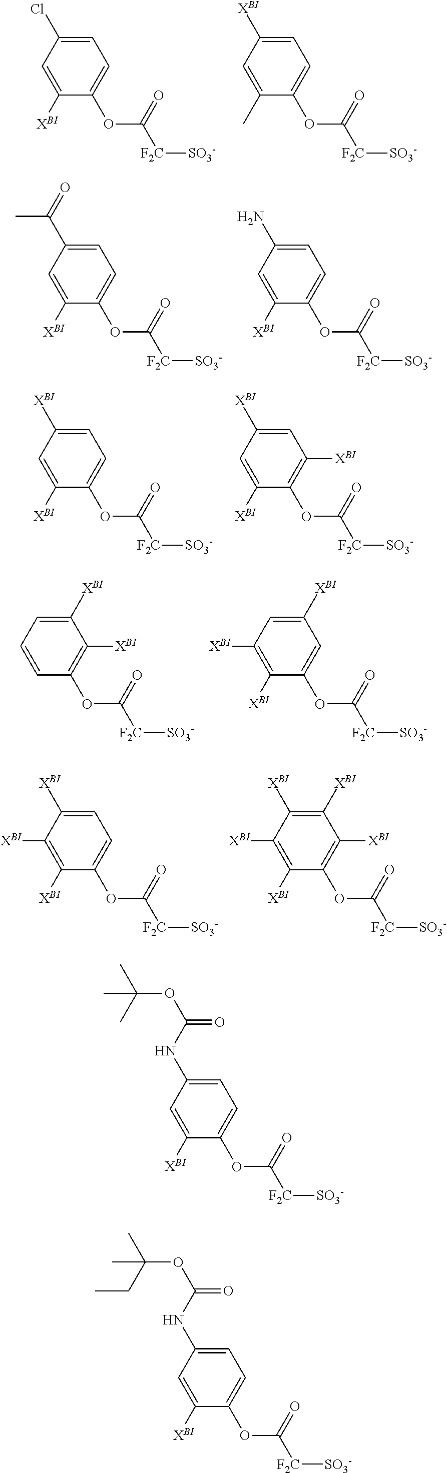

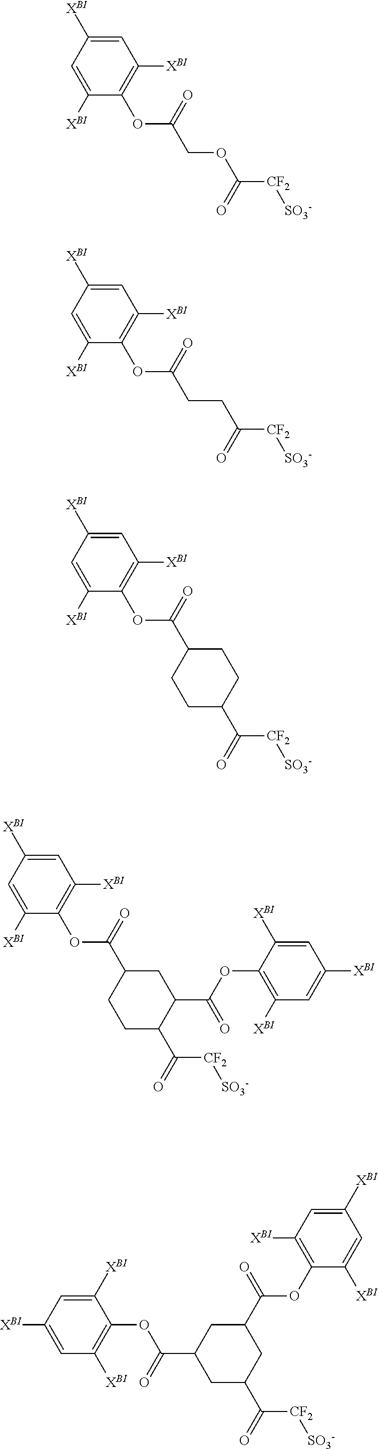

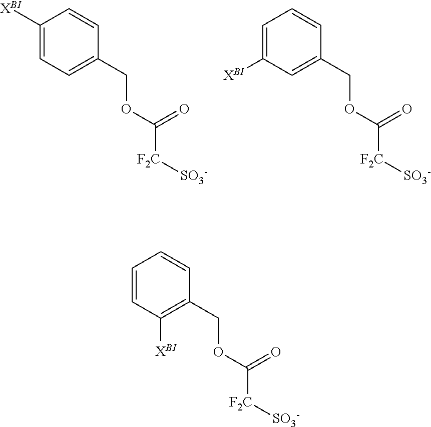

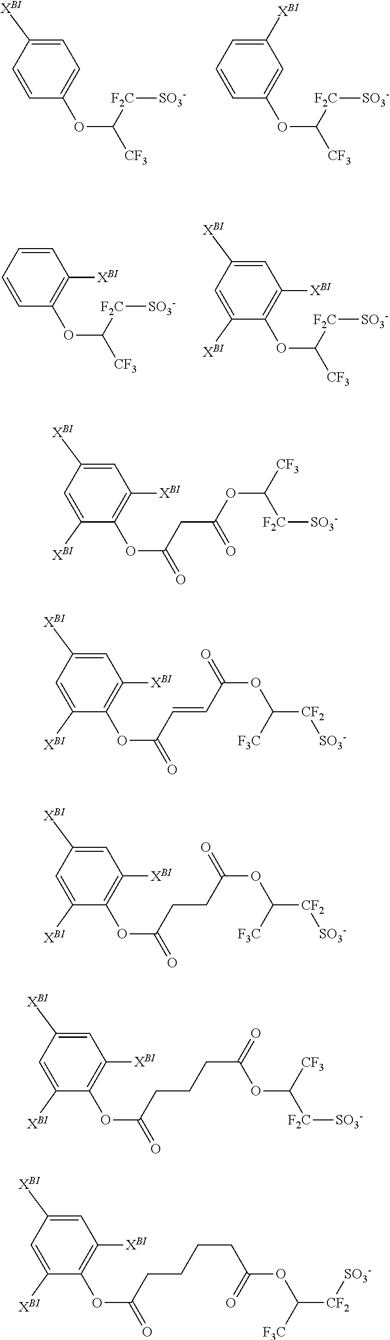

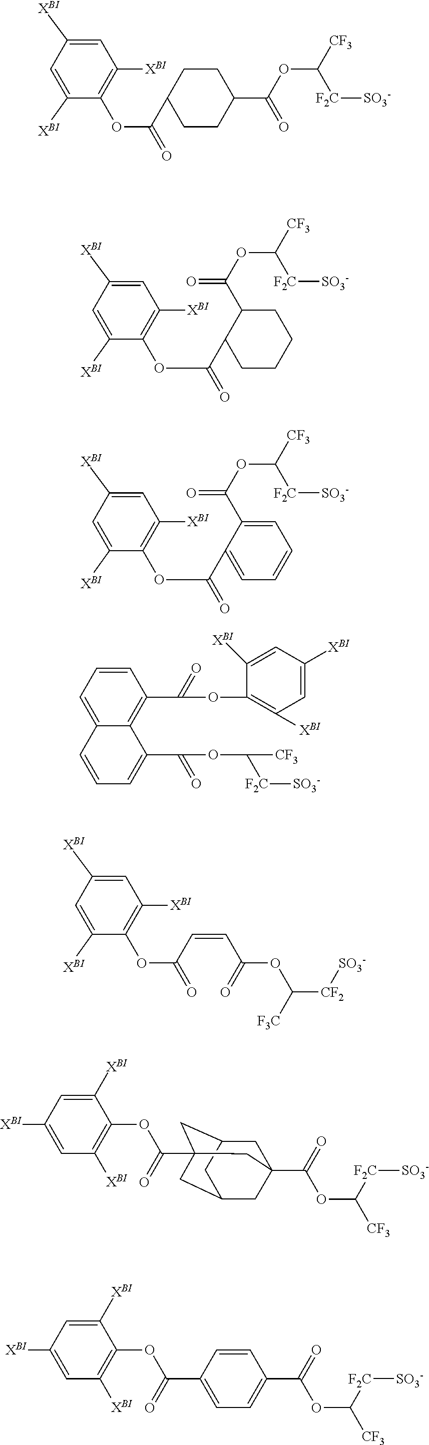

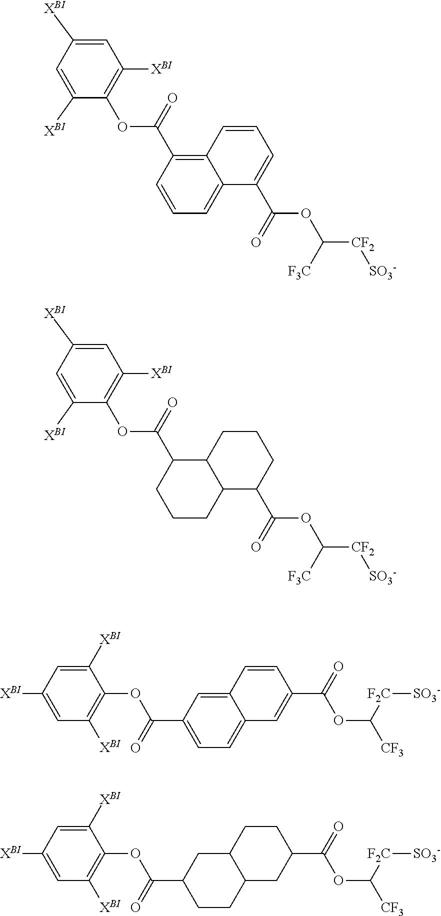

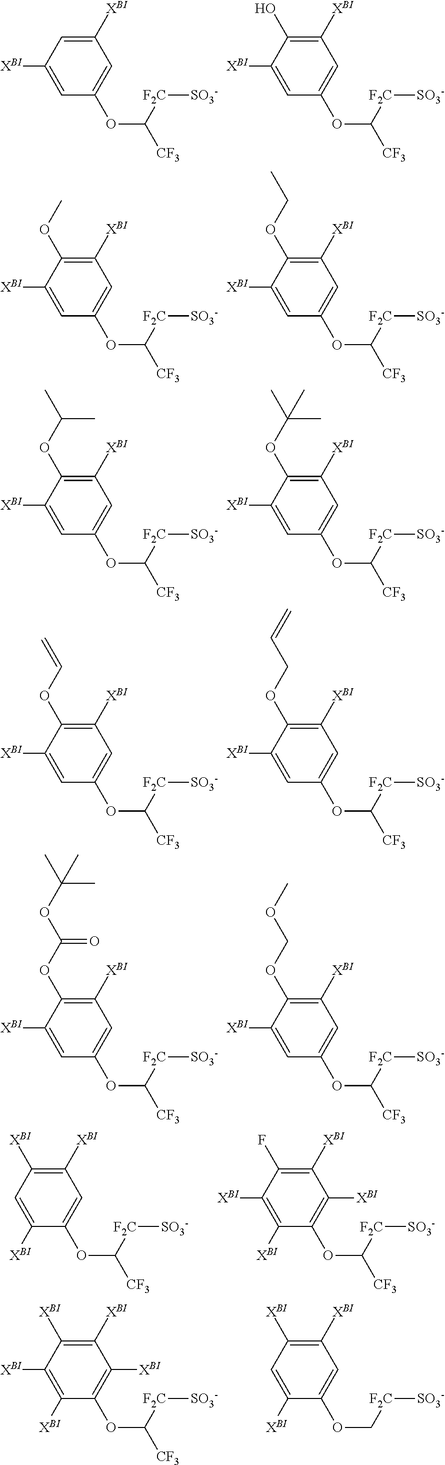

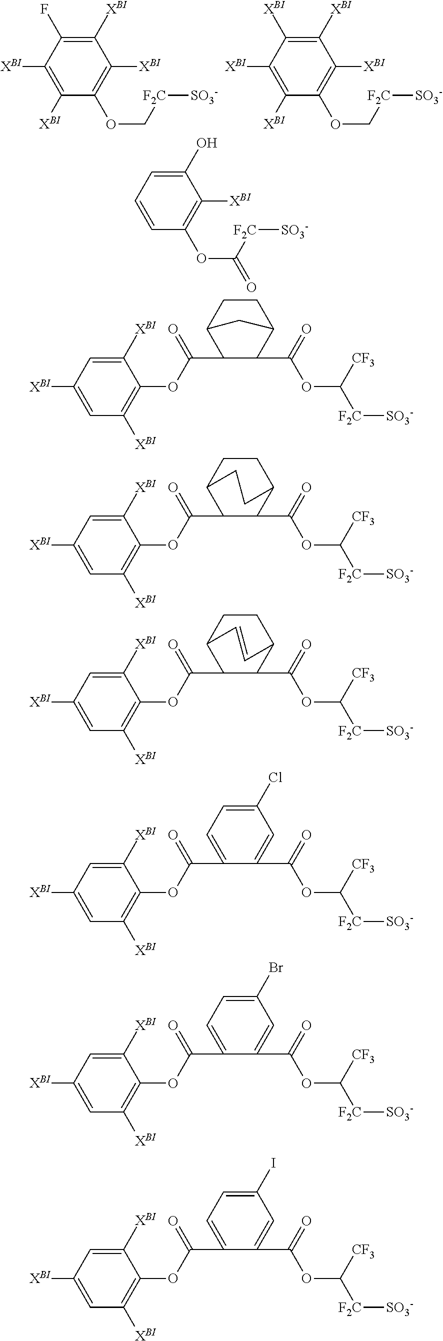

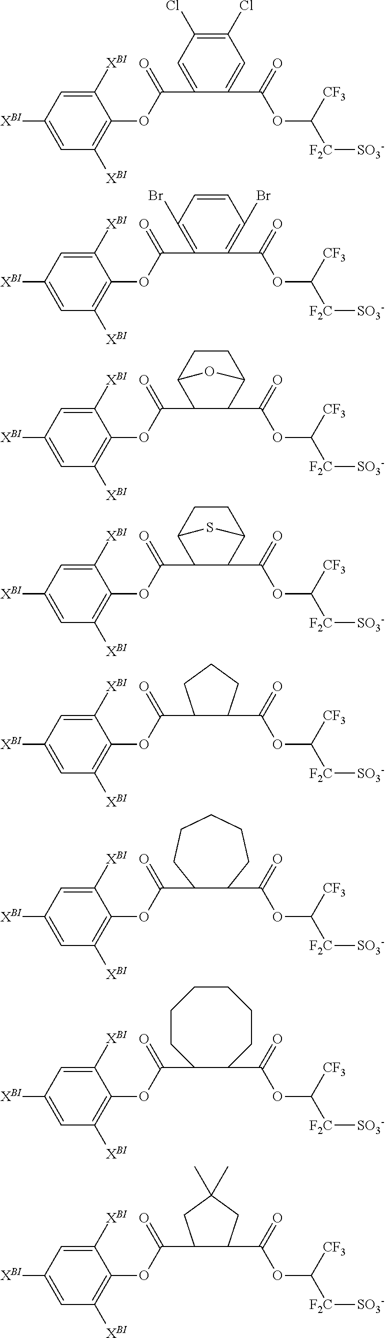

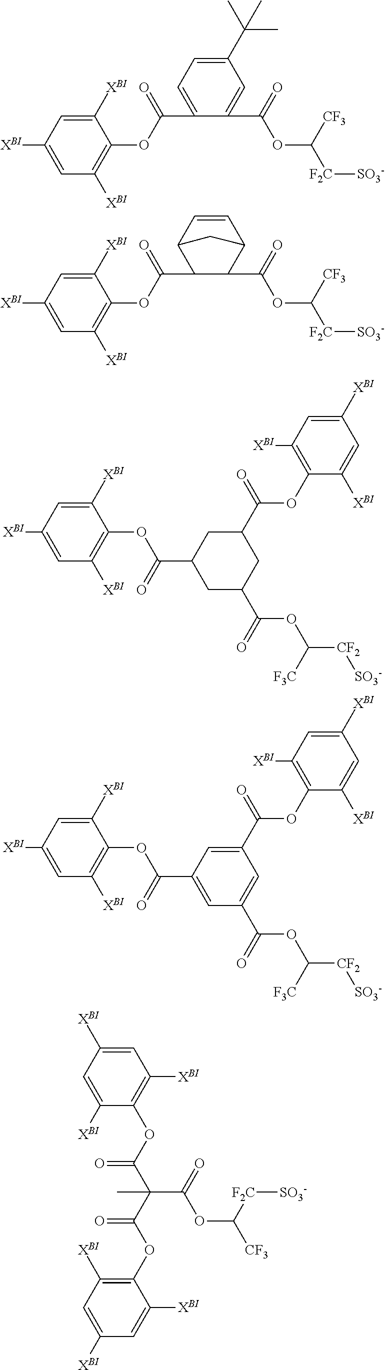

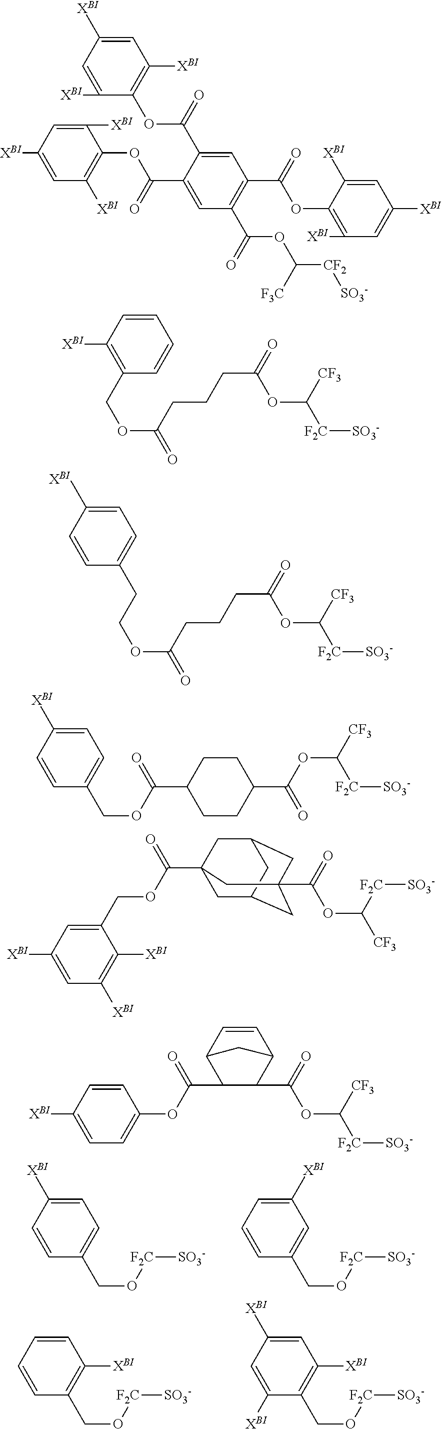

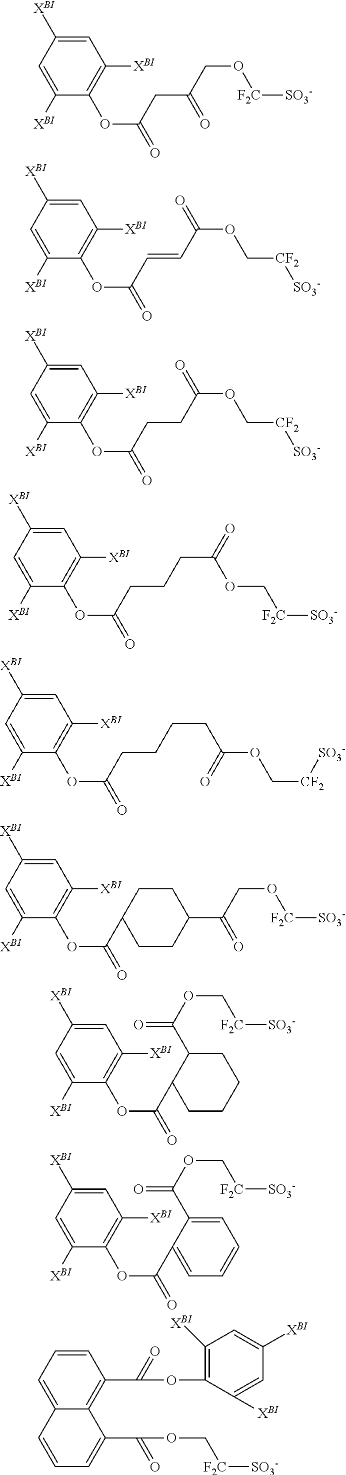

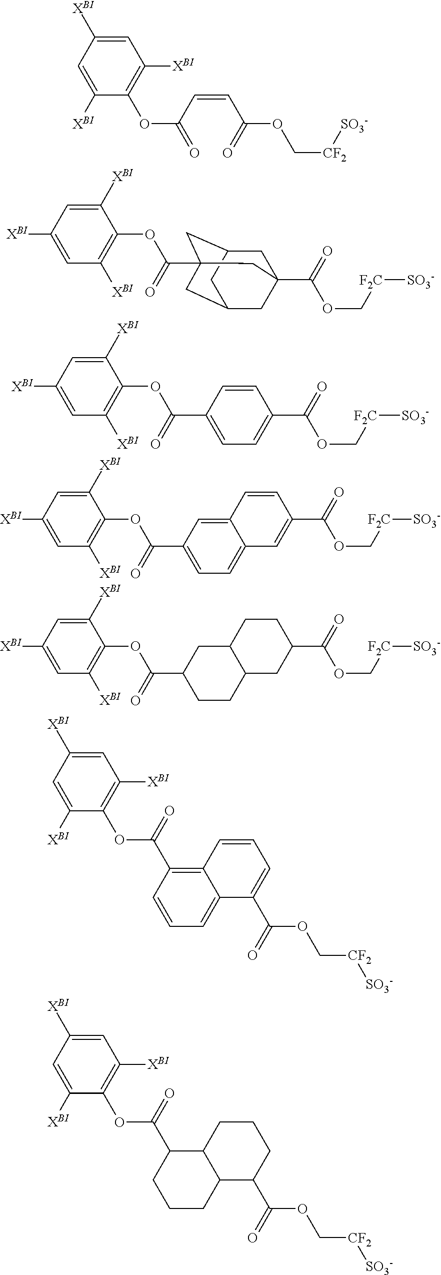

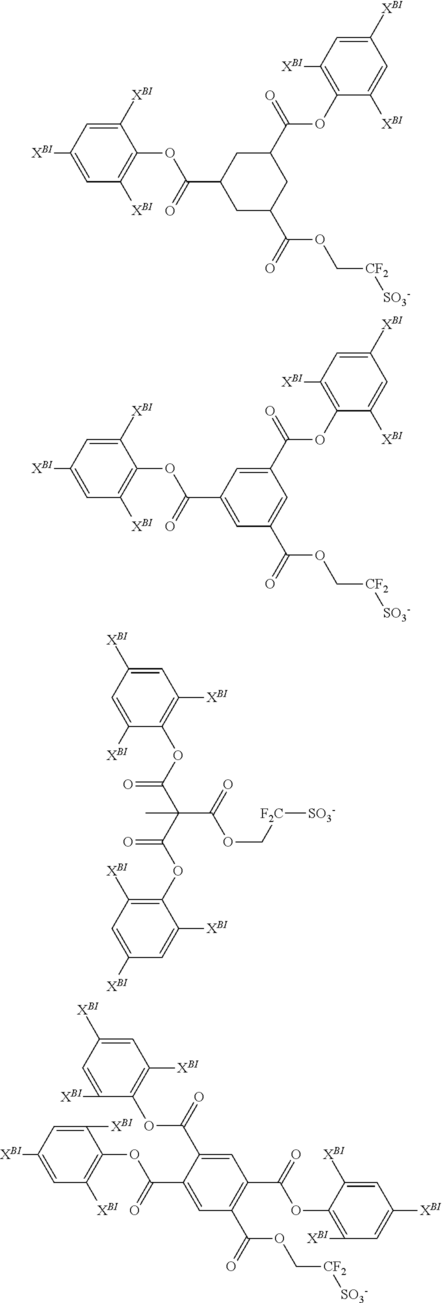

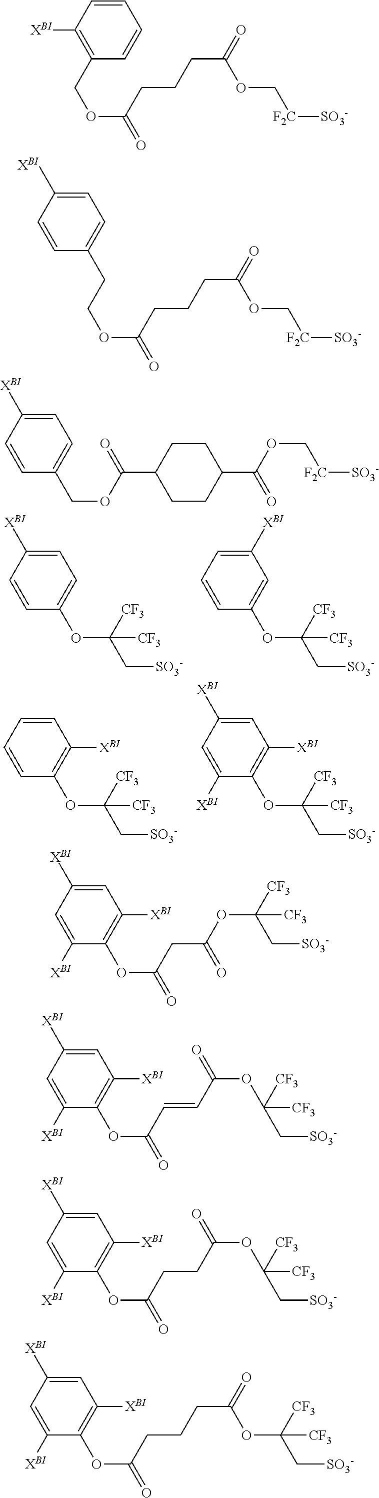

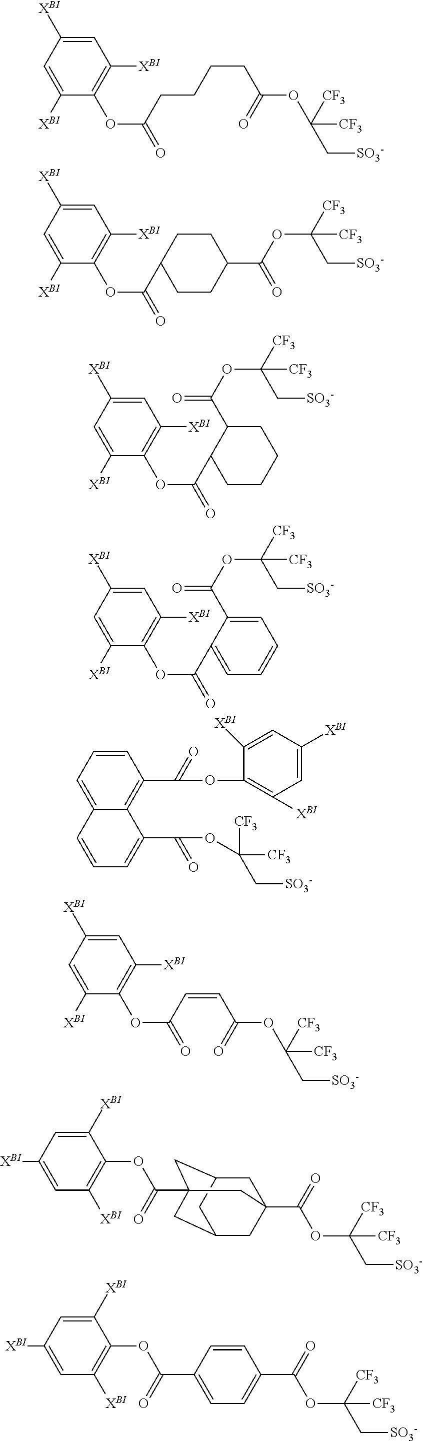

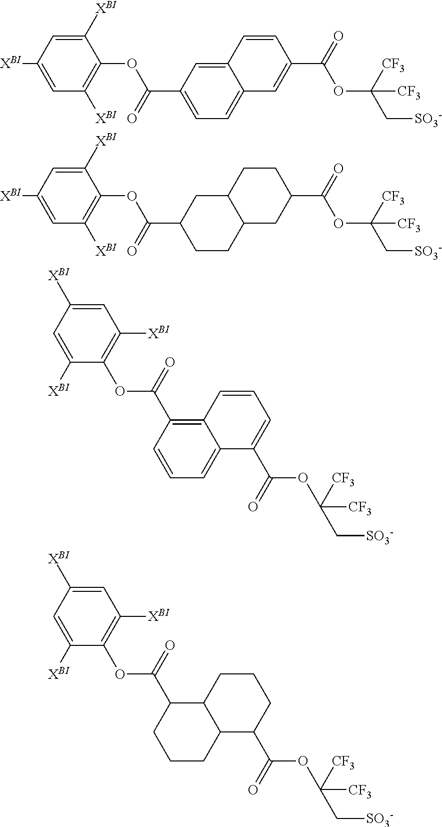

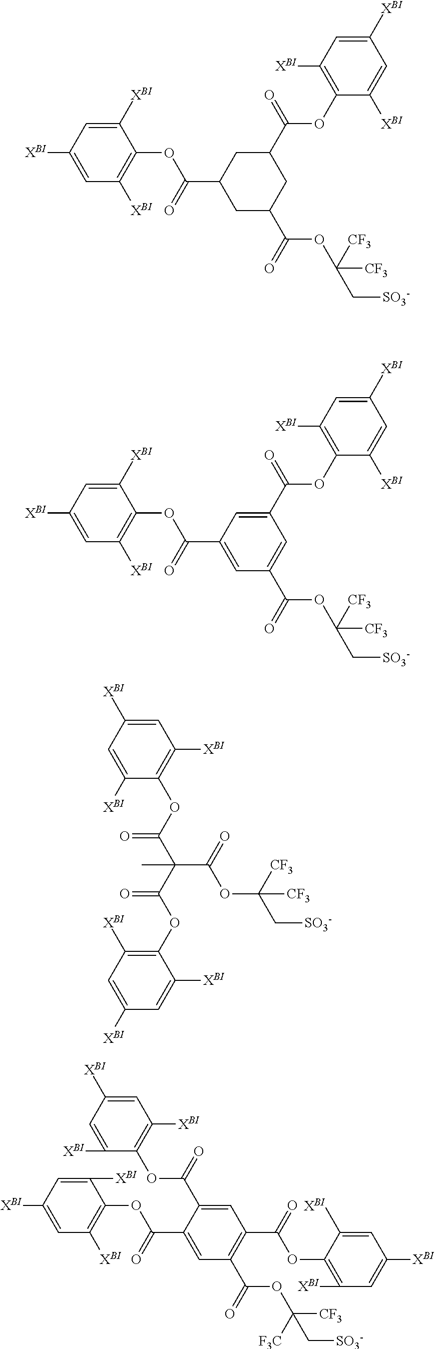

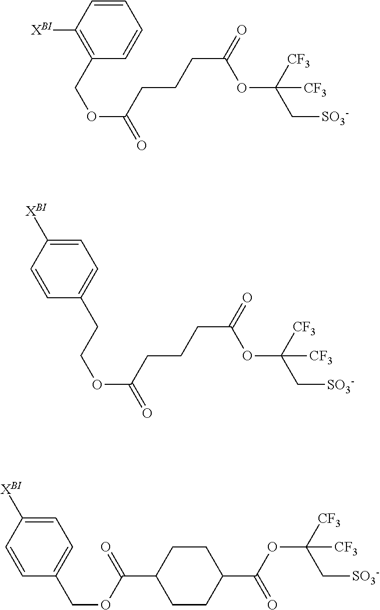

As the non-nucleophilic counter ion, a fluorobenzenesulfonic acid anion attached to an aromatic group containing an iodine atom described in JP 6648726, an anion having a mechanism of decomposition by an acid described in WO 2021/200056 A or JP-A 2021-70692, an anion having a cyclic ether group described in JP-A 2018-180525 or JP-A 2021-35935, and an anion described in JP-A 2018-92159 can also be used.

As the non-nucleophilic counter ion, an anion of a bulky benzenesulfonic acid derivative free of fluorine atoms described in JP-A 2006-276759, JP-A 2015-117200, JP-A 2016-65016, or JP-A 2019-202974, and a benzenesulfonic acid anion or an alkylsulfonic acid anion free of fluorine atoms attached to an aromatic group containing an iodine atom described in JP 6645464 can also be used.

As the non-nucleophilic counter ion, further an anion of a bissulfonic acid described in JP-A 2015-206932, an anion having a sulfonate on one side and a sulfonamide or sulfonimide differing therefrom on the other side described in WO 2020/158366 A, and an anion having a sulfonate on one side and a carboxylate on the other side described in JP-A 2015-24989 can also be used.

In the formulae (c2) and (c3), d1 and d2 are each independently 0, 1, 2, or 3, and preferably 1.

In the formula (c4), e1 is 0 or 1, e2 is 0, 1, 2, 3, or 4, and e3 is 0, 1, 2, 3, or 4, meeting 0≤e2+e3≤4 when e1 is 0, and 0≤e2+e3≤6 when e1 is 1.

In the formulae (c2), (c3), and (c4), L¹ is a single bond, an ether bond, an ester bond, a carbonyl group, a sulfonate ester bond, a sulfonamide bond, a carbonate bond, or a carbamate bond, Among them, from the aspect of synthesis, an ether bond, an ester bond, and a carbonyl group are preferred, and an ester bond and a carbonyl group are more preferred.

In the formula (c2), Rf¹ and Rf² are each independently a fluorine atom or a C₁-C₆ fluorinated saturated hydrocarbyl group. Among them, Rf¹ and Rf² are each preferably a fluorine atom in order to increase the acid strength of the generated acid. Rf³ and Rf⁴ are each independently a hydrogen atom, a fluorine atom, or a C₁-C₆ fluorinated saturated hydrocarbyl group. Among them, at least one of Rf³ and Rf⁴ is preferably a trifluoromethyl group for improving the solvent solubility.

In the formula (c3), Rf⁵ and Rf⁶ are each independently a hydrogen atom, a fluorine atom, or a C₁-C₆ fluorinated saturated hydrocarbyl group. It is excluded that all Rf⁵ and Rf⁶ are a hydrogen atom at the same time. Among them, at least one of Rff and Rf⁶ is preferably a trifluoromethyl group for improving the solvent solubility.

In the formula (c4), Rf⁷ is a fluorine atom, a C₁-C₆ fluorinated alkyl group, a C₁-C₆ fluorinated alkoxy group, or a C₁-C₆ fluorinated alkylthio group. As Rf⁷, a fluorine atom, a trifluoromethyl group, a difluoromethyl group, a trifluoromethoxy group, a difluoromethoxy group, a trifluoromethylthio group, or a difluoromethylthio group is preferred, and a fluorine atom, a trifluoromethyl group, or a trifluoromethoxy group is more preferred. When f is 2, 3, or 4, a plurality of Rf⁷'s may be identical or different. In the formula (c4), R³³ is a halogen atom other than a fluorine atom, or a C₁-C₂₀ hydrocarbyl group which may contain a heteroatom. The hydrocarbyl group may be saturated or unsaturated and straight, branched, or cyclic. Specific examples thereof are as exemplified above for the hydrocarbyl group of R¹ in the description of the formula (1), but are not limited thereto. When e3 is 2, 3, or 4, a plurality of R³³'s may be identical or different.

When e3 is 2, 3, or 4, a plurality of R³³'s may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. Some or all hydrogen atoms in the ring may be substituted with a group containing a heteroatom such as an oxygen atom, a sulfur atom, a nitrogen atom, or a halogen atom, and some —CH₂— in the ring may be replaced by a group containing a heteroatom such as an oxygen atom, a sulfur atom, or a nitrogen atom, so that the ring may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Specific examples of the anion of the repeat unit (c2) are shown below, but not limited thereto. In the following formulae. R^{A} is as defined above, and Me is a methyl group.

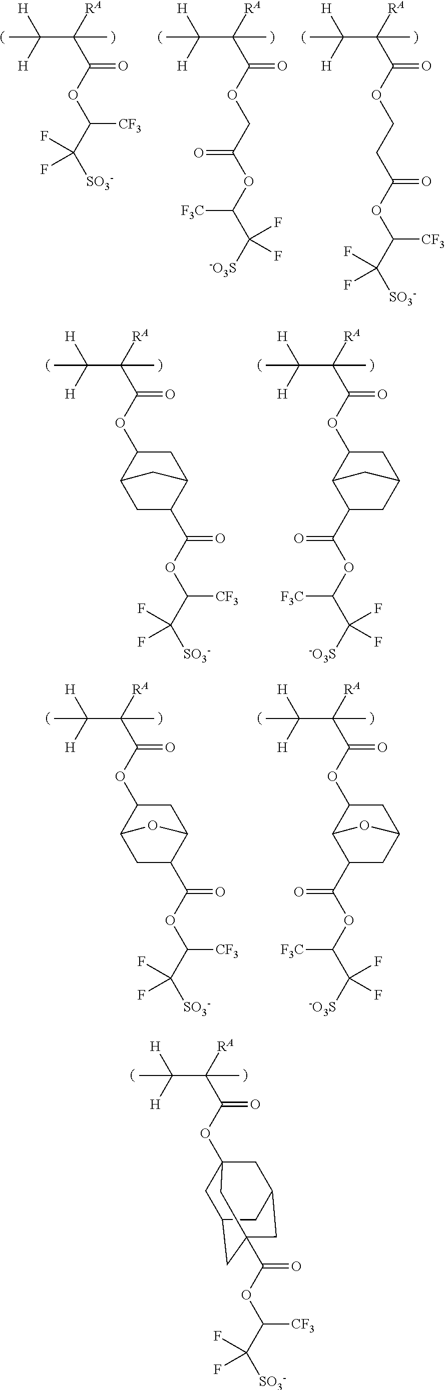

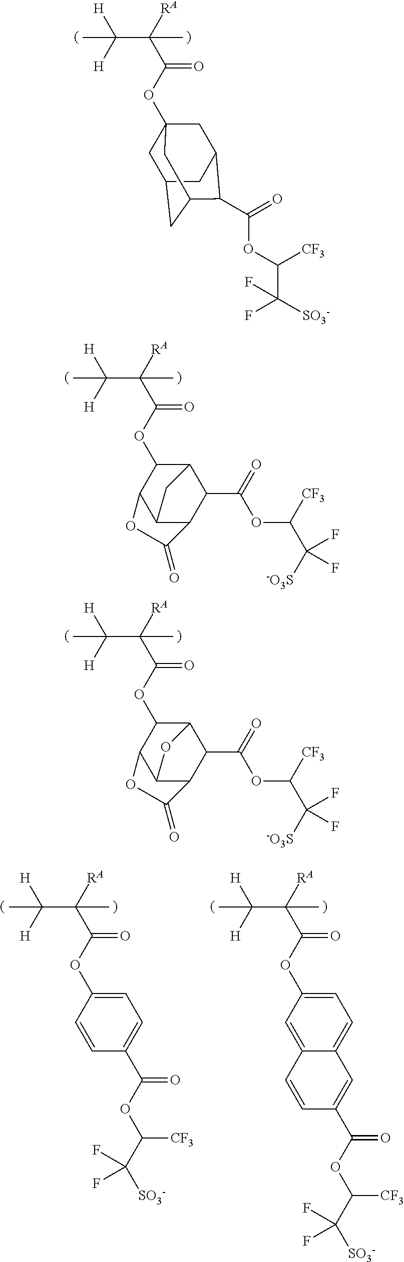

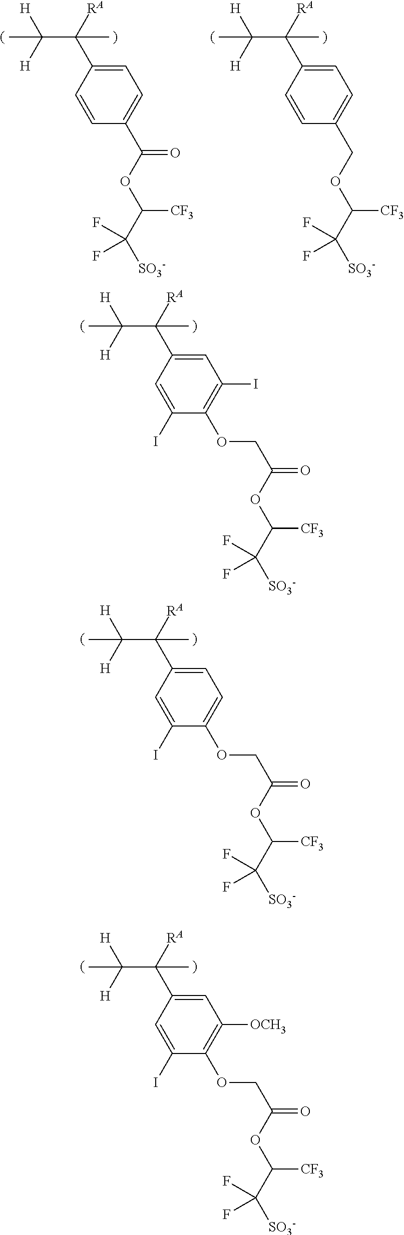

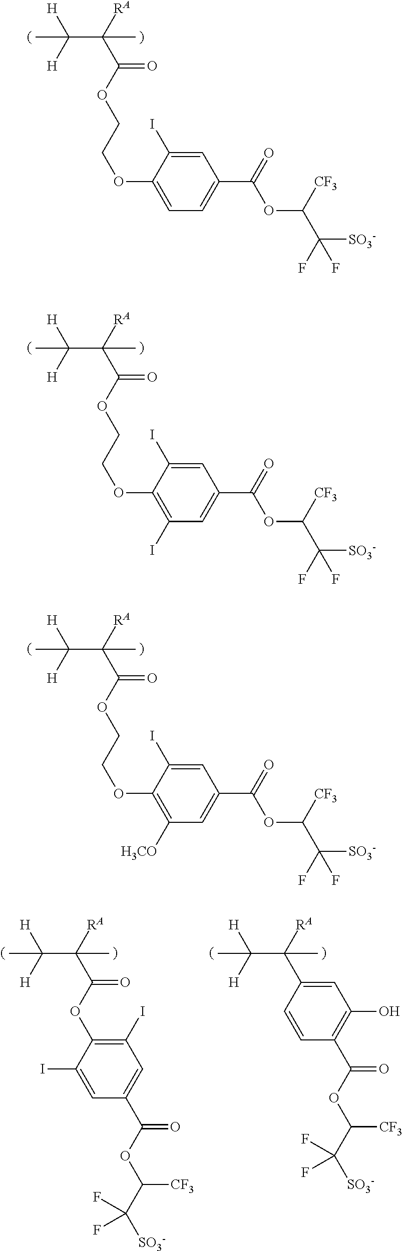

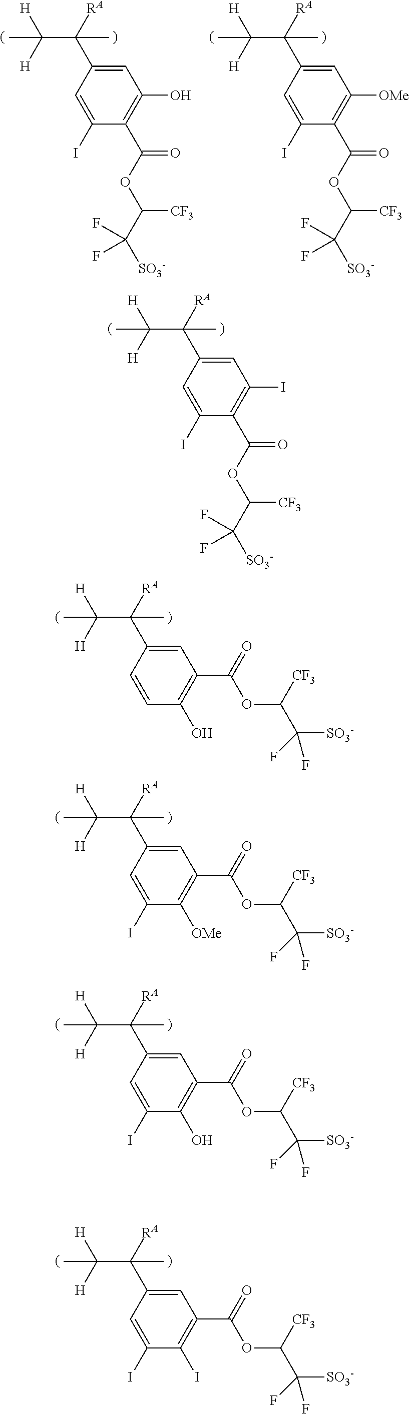

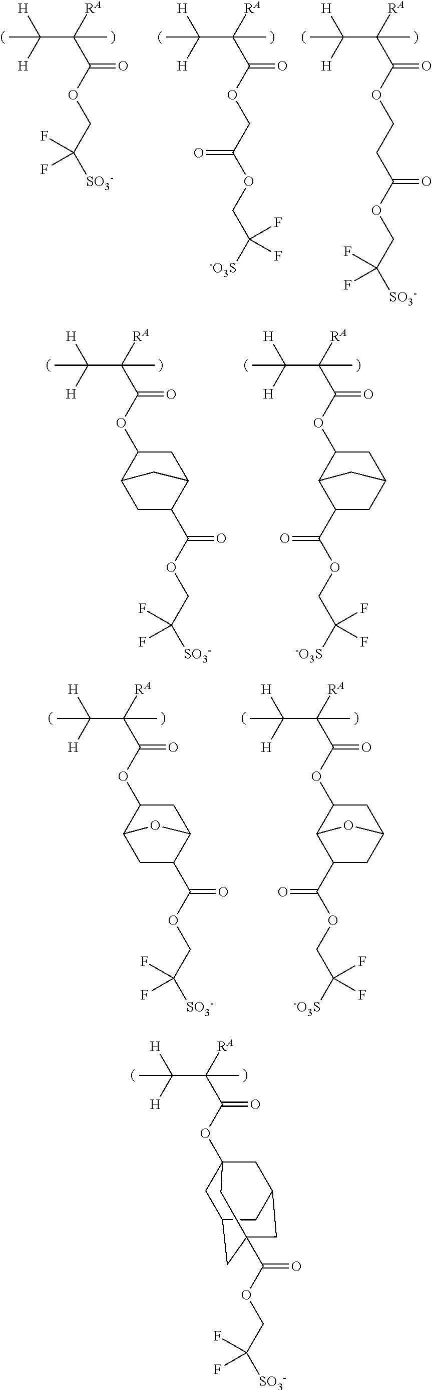

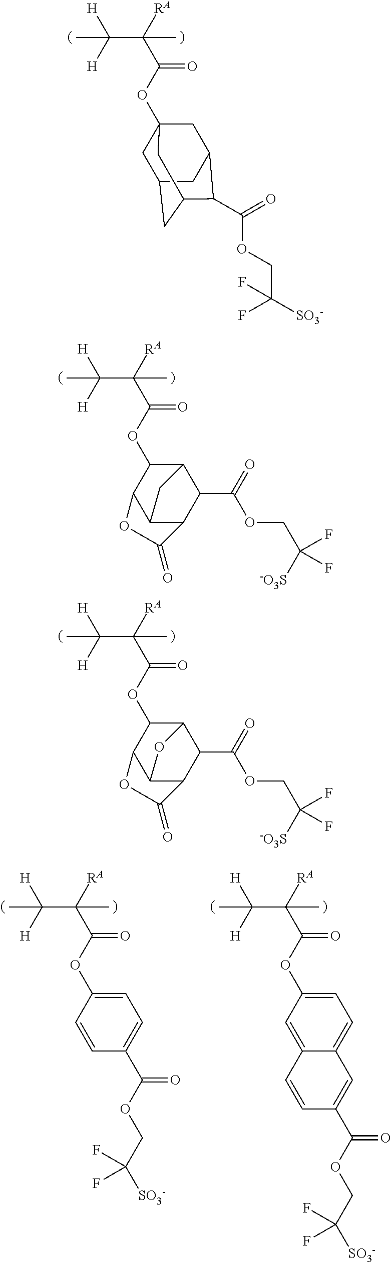

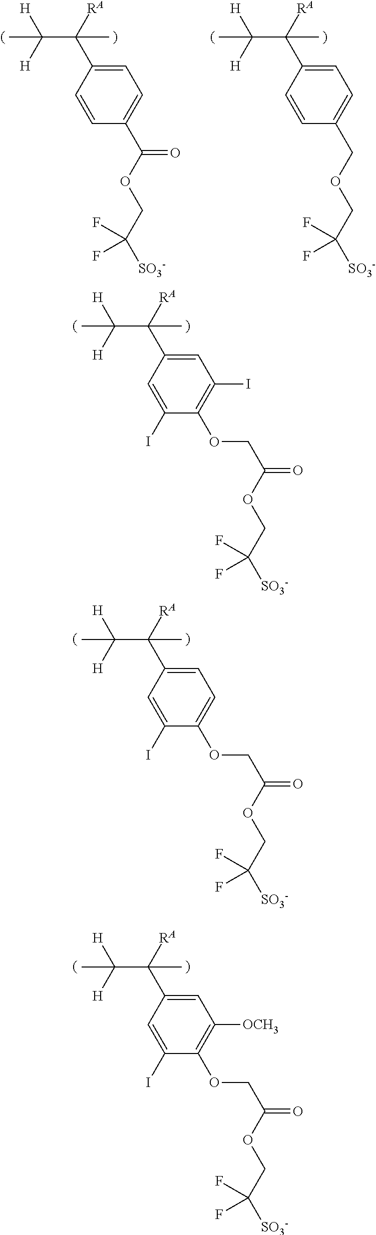

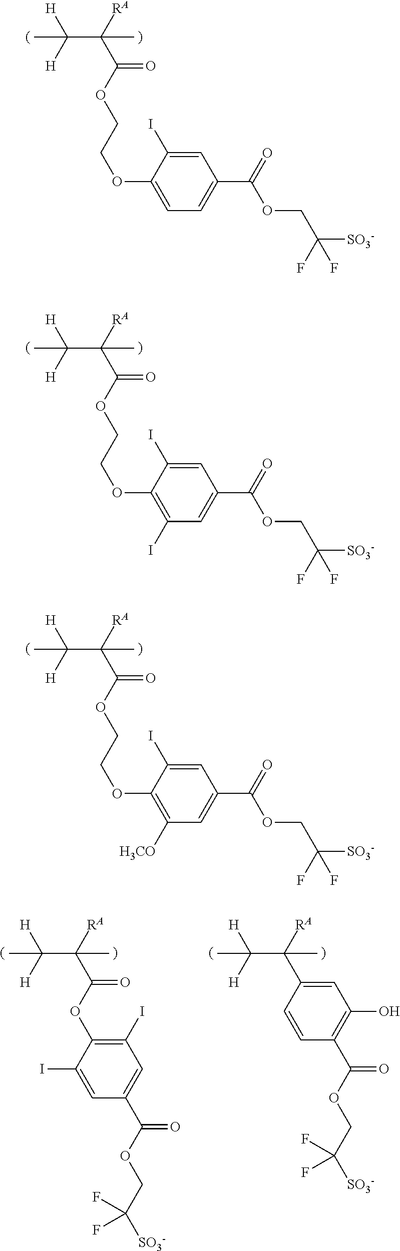

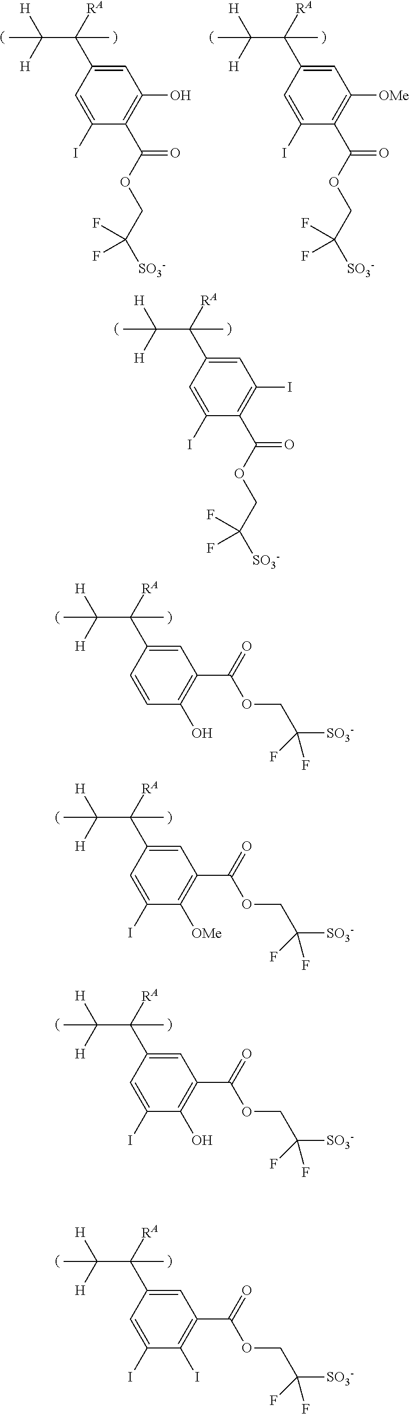

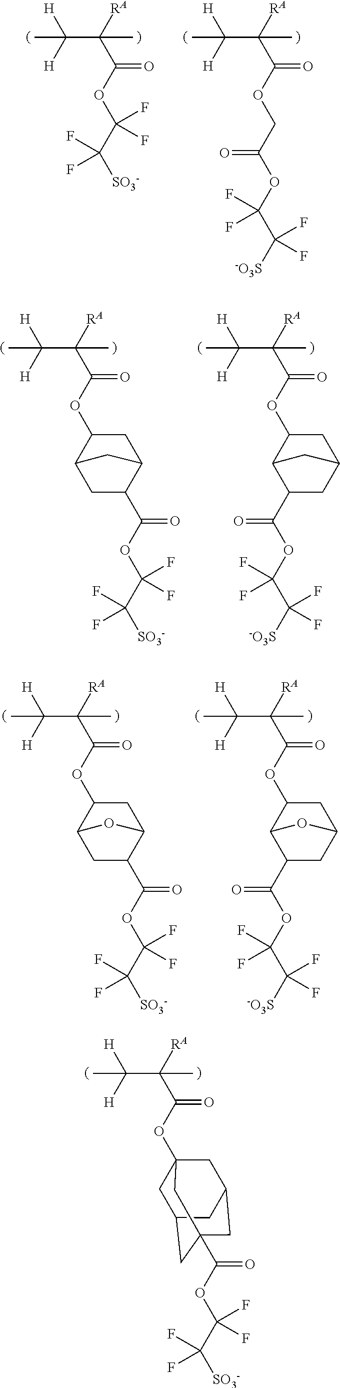

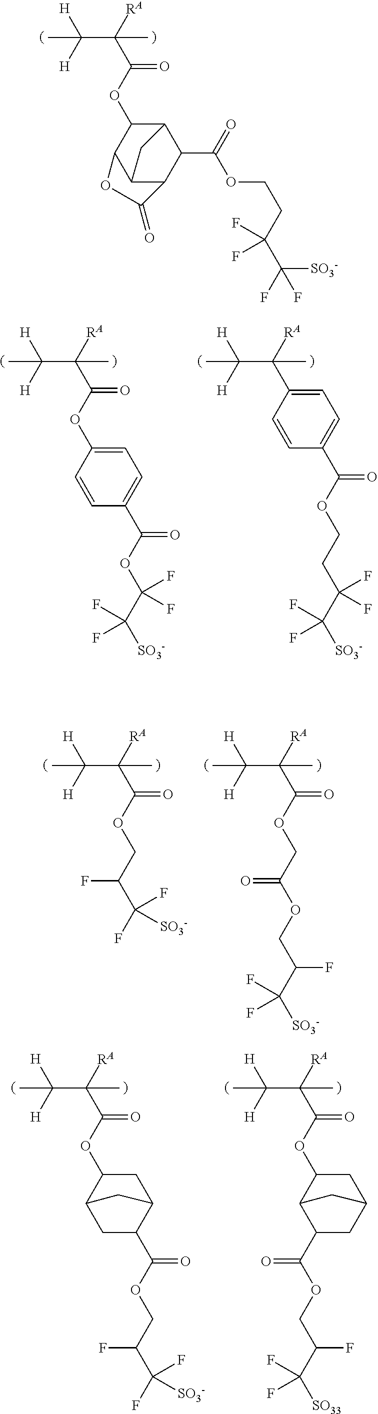

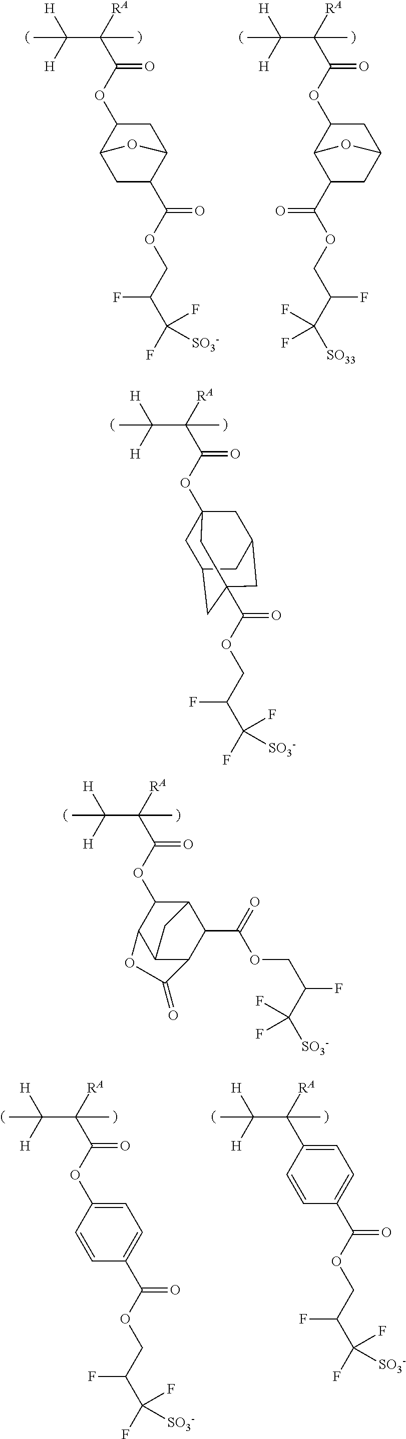

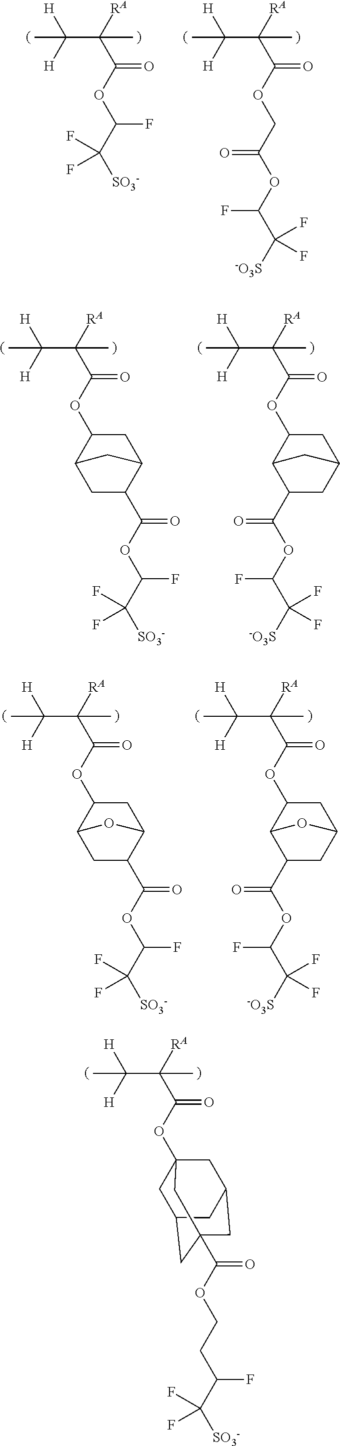

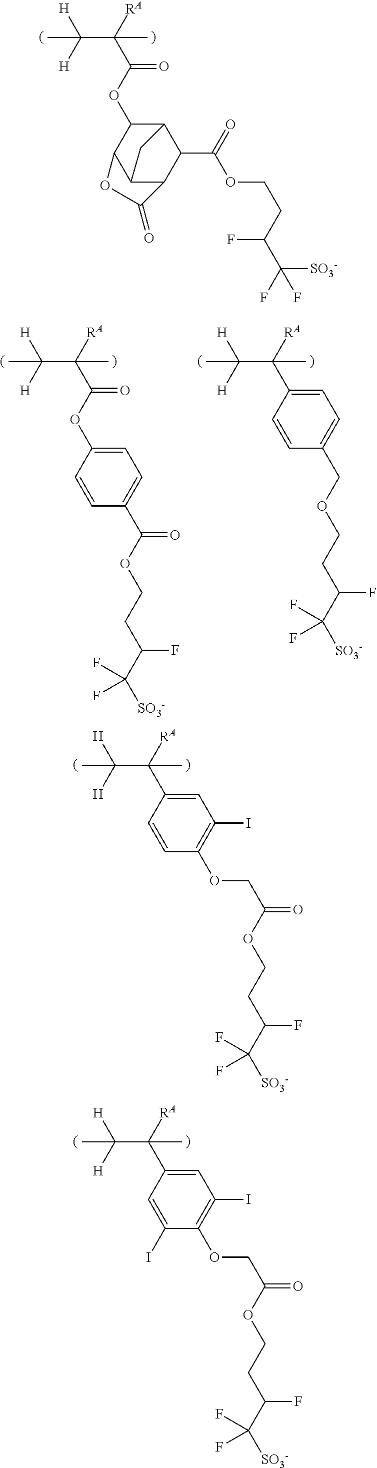

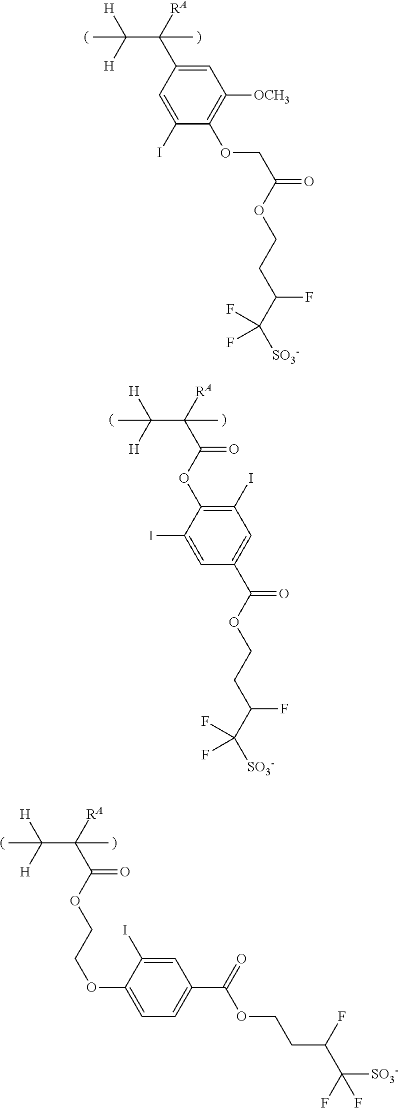

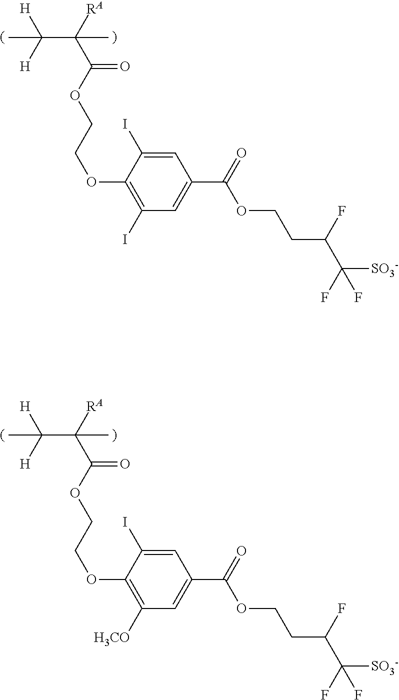

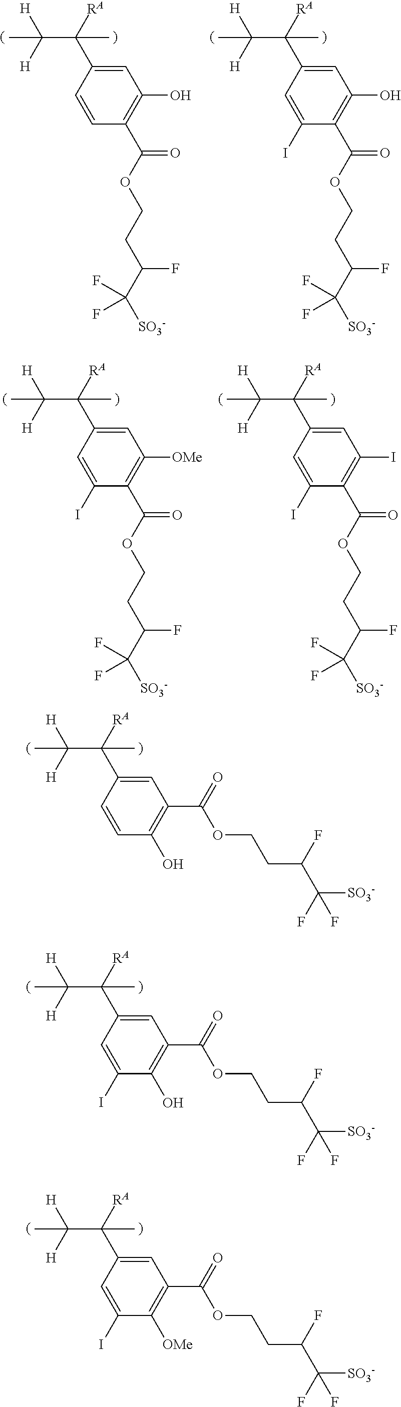

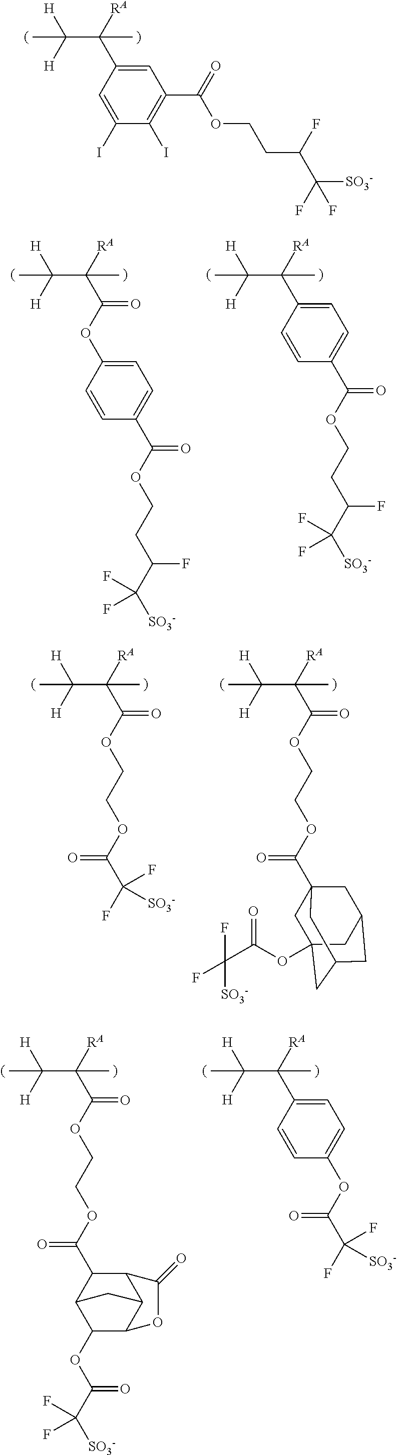

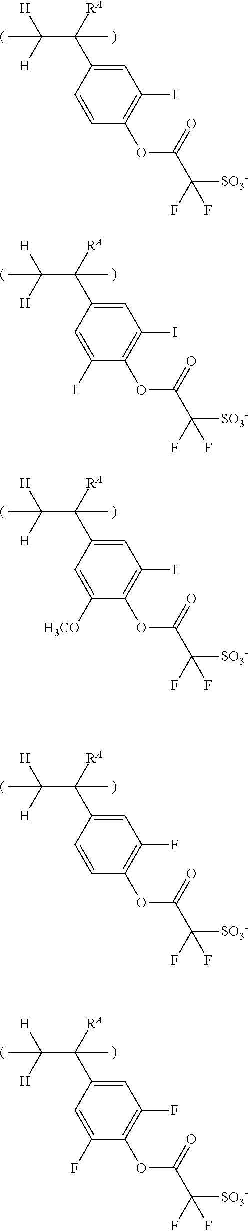

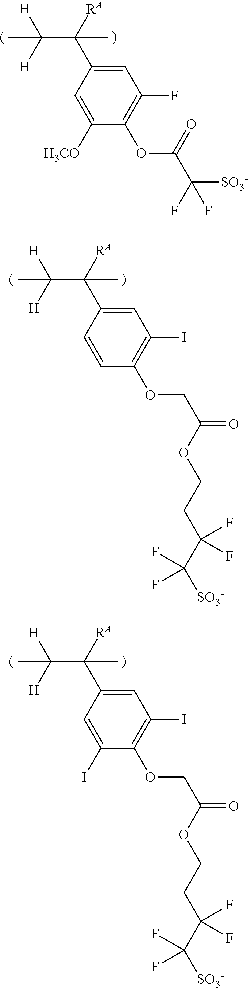

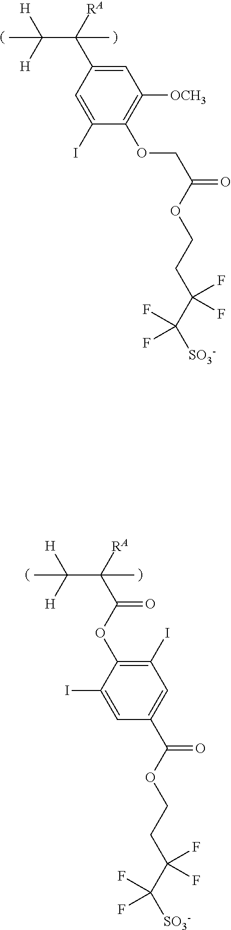

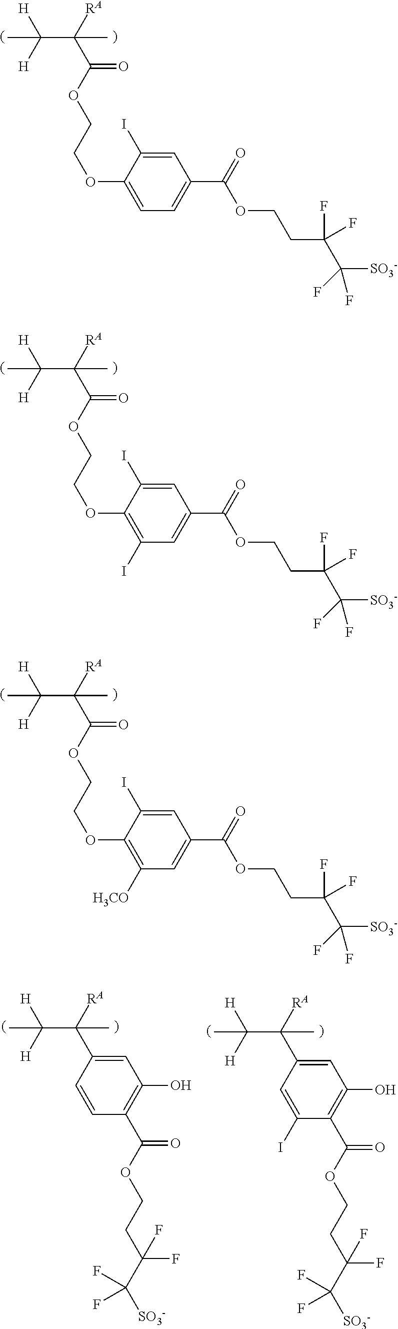

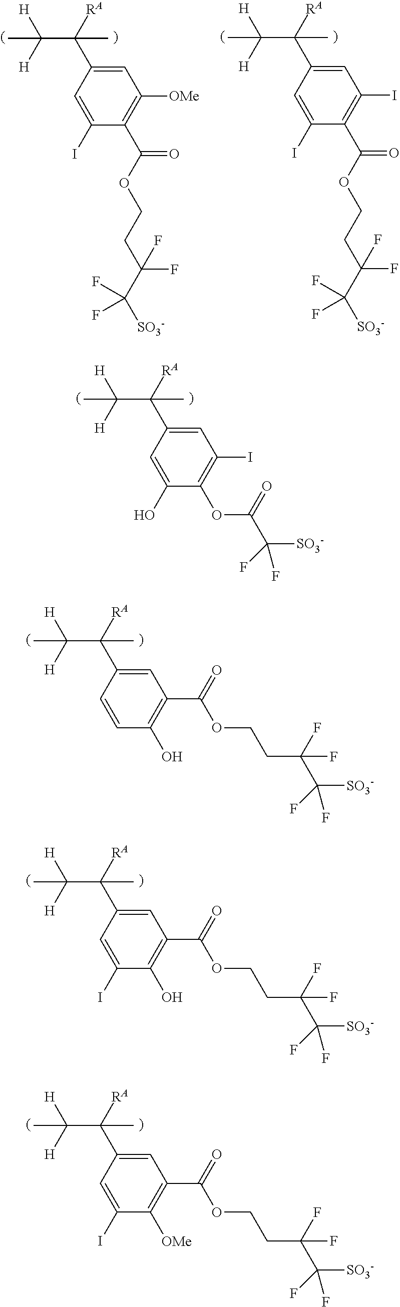

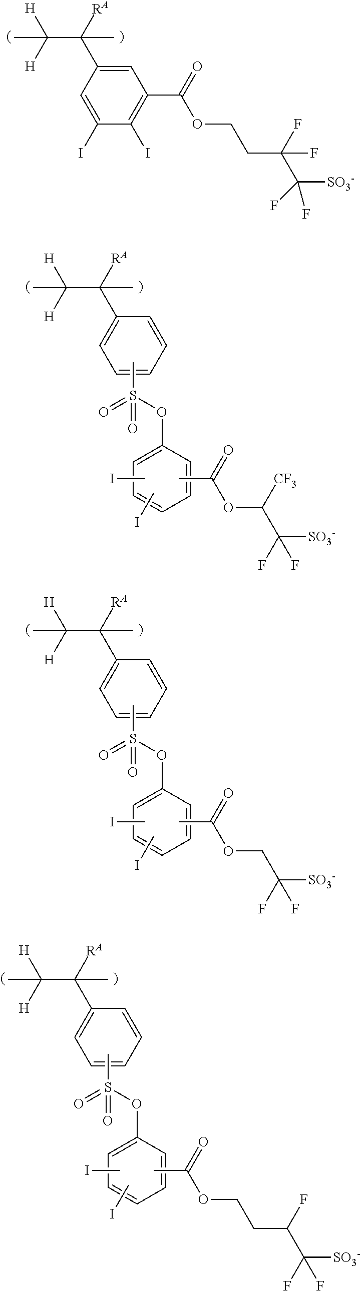

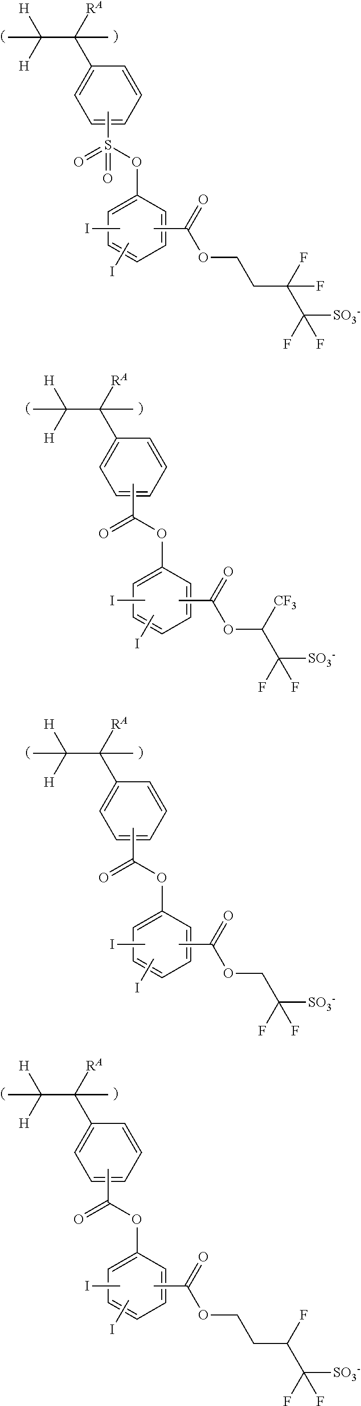

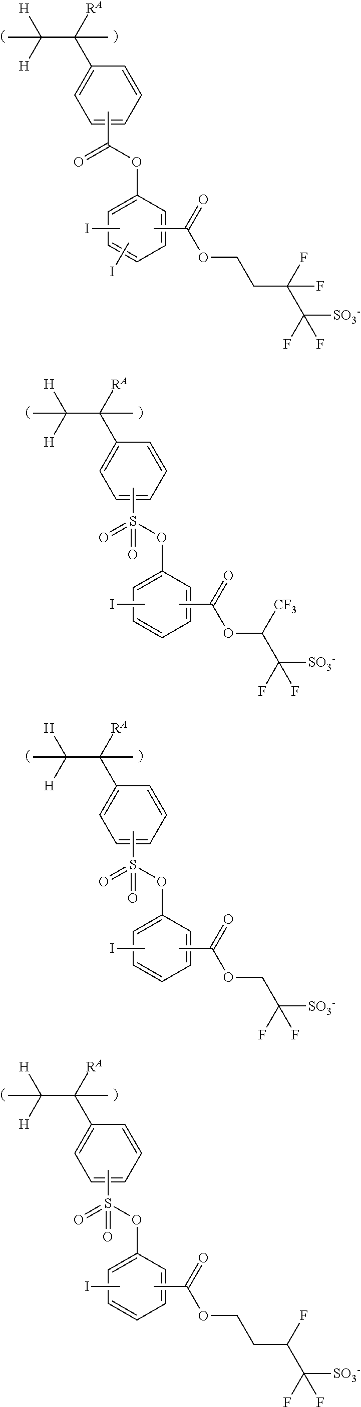

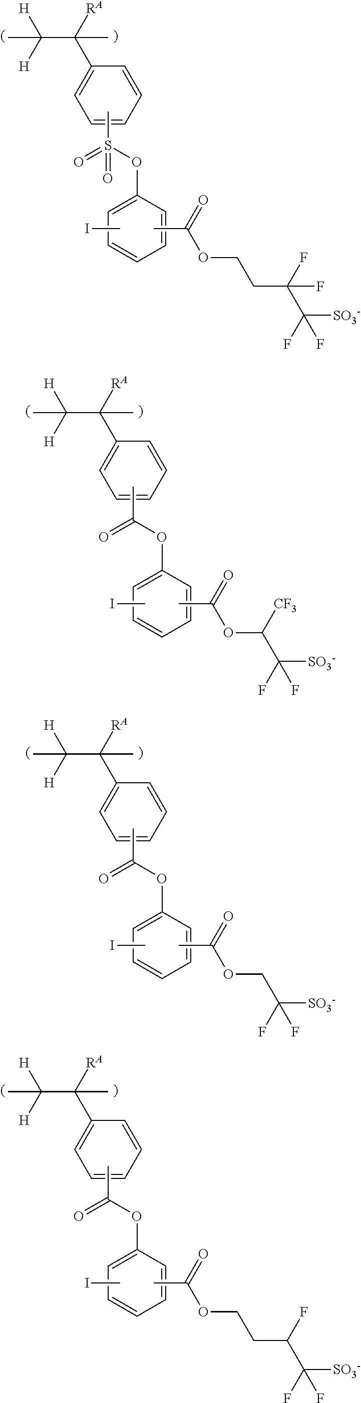

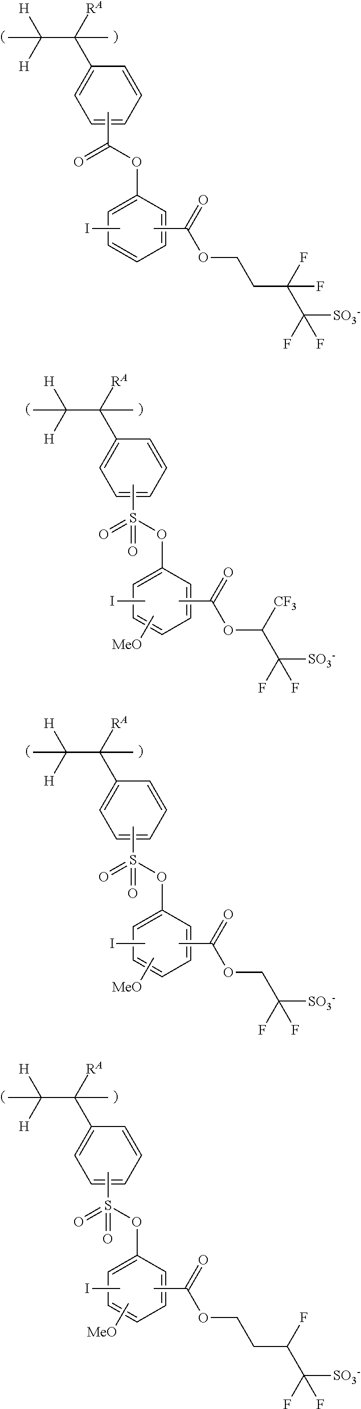

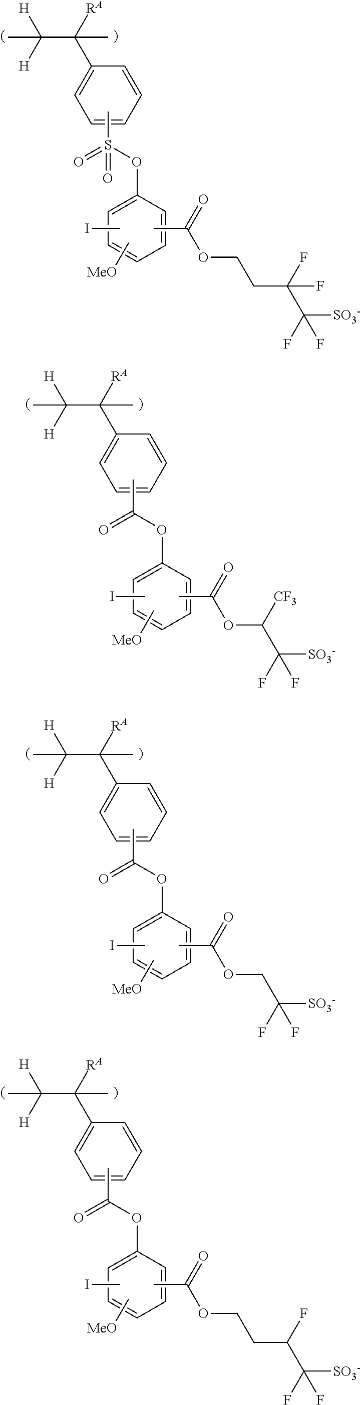

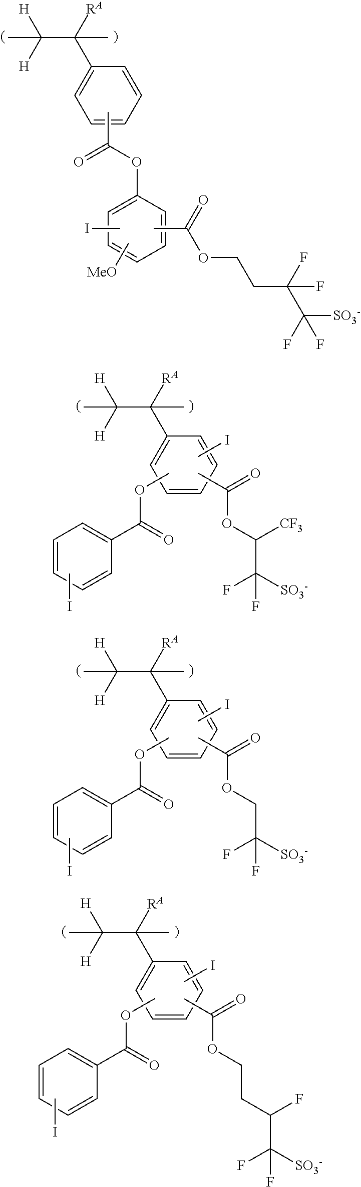

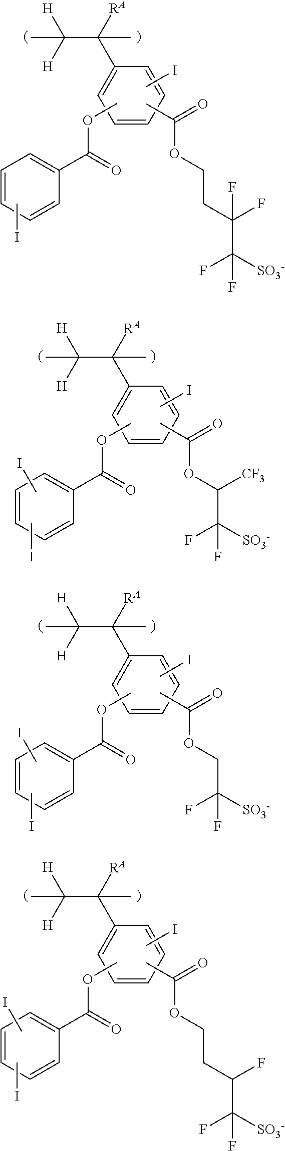

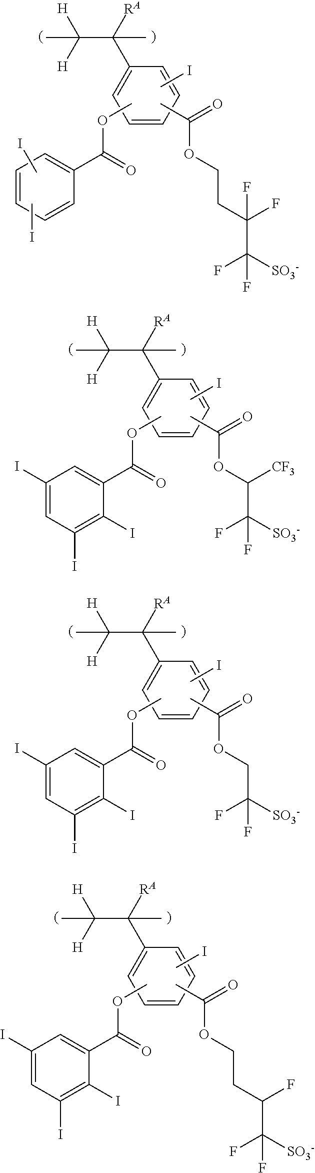

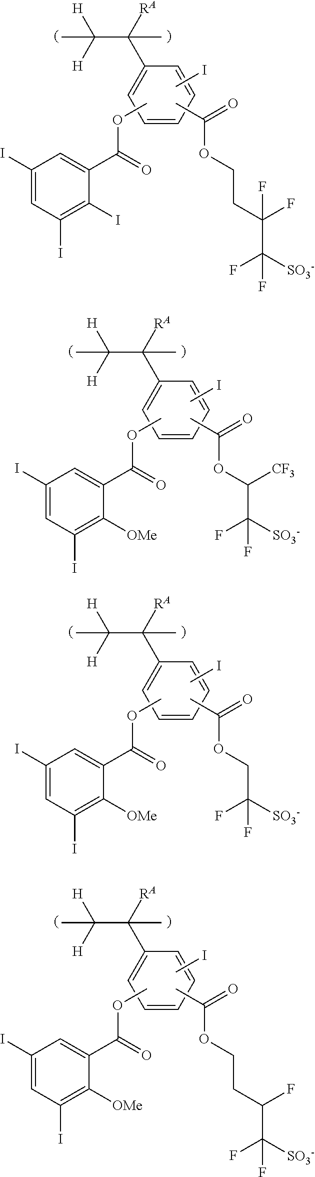

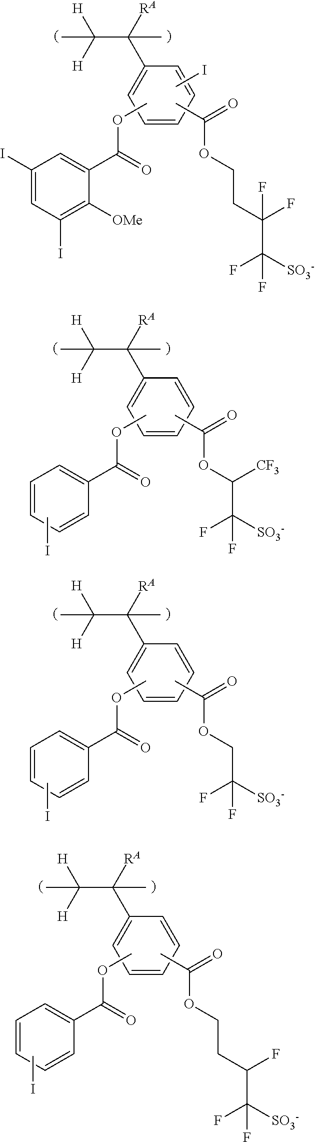

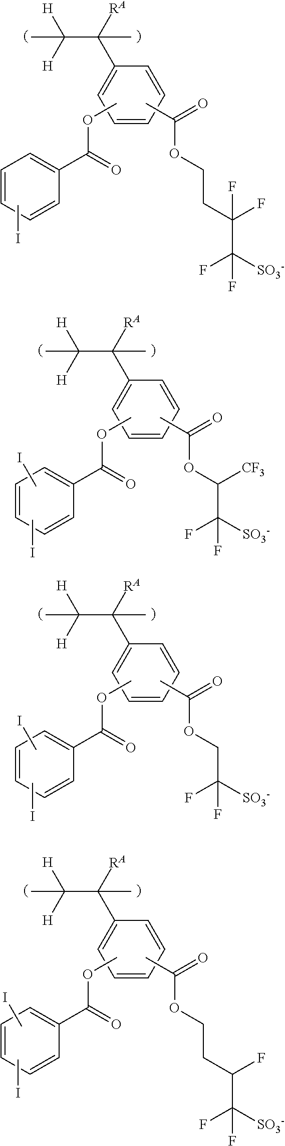

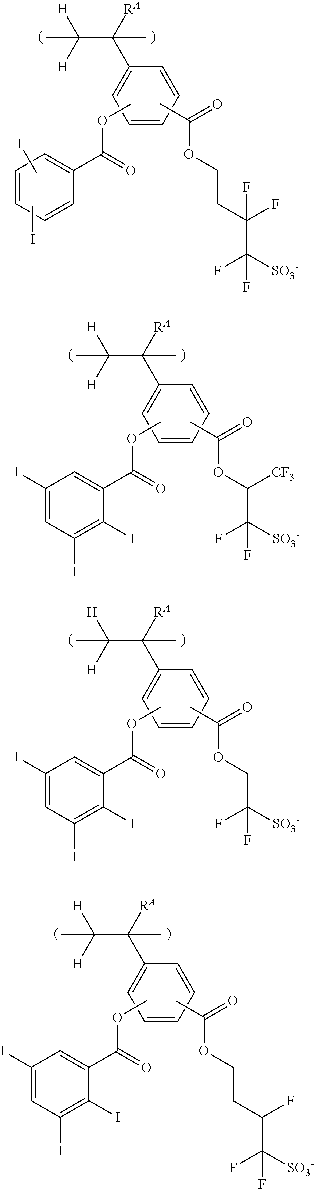

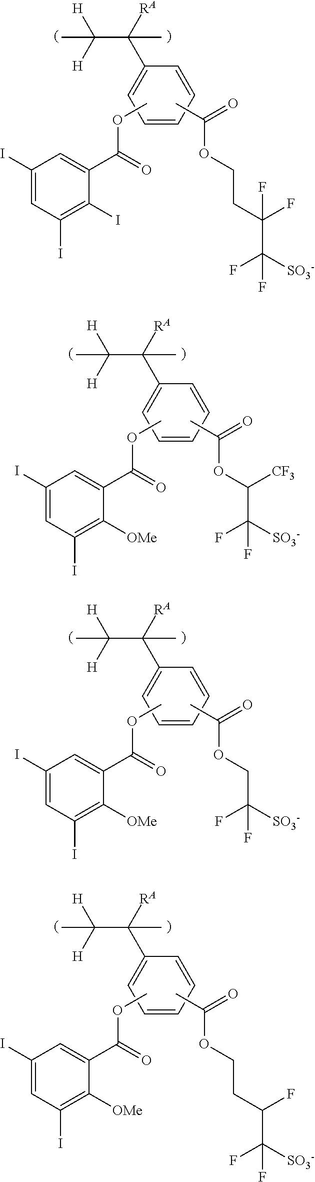

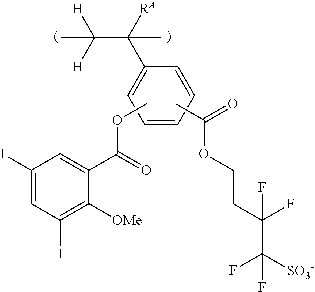

Specific examples of the anion of the repeat unit (c3) are shown below, but not limited thereto. In the following formulae. R^{A} is as defined above.

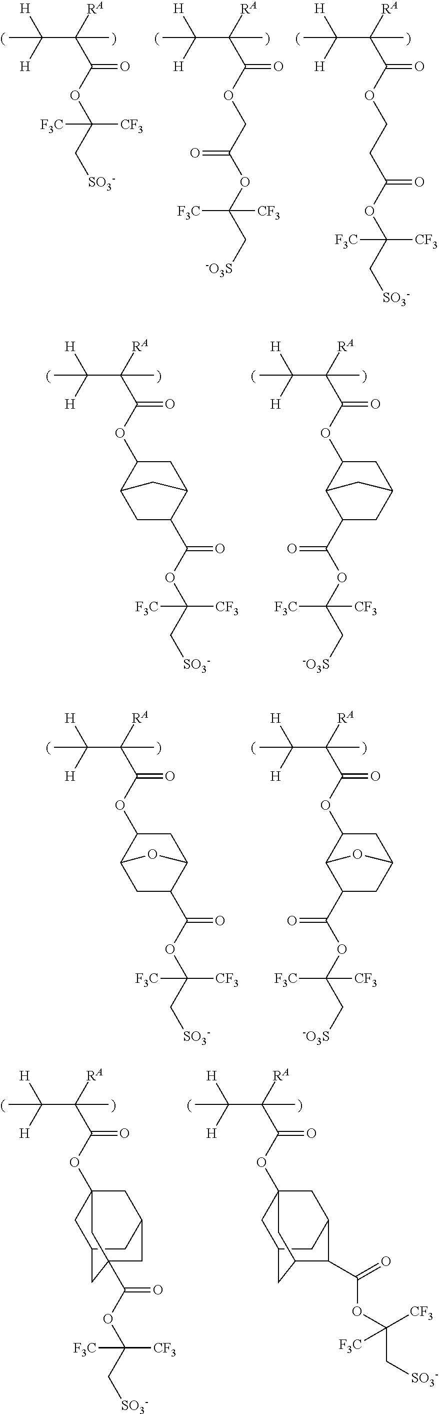

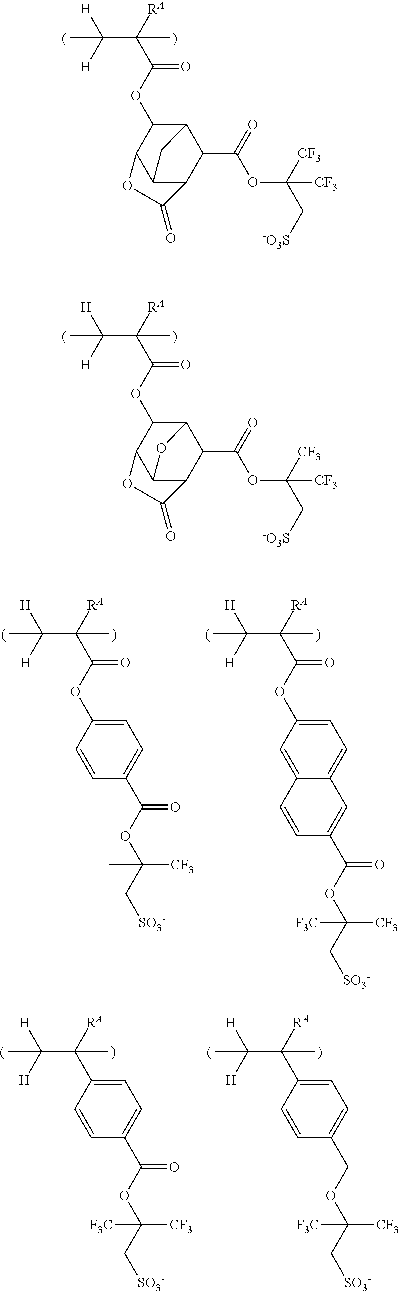

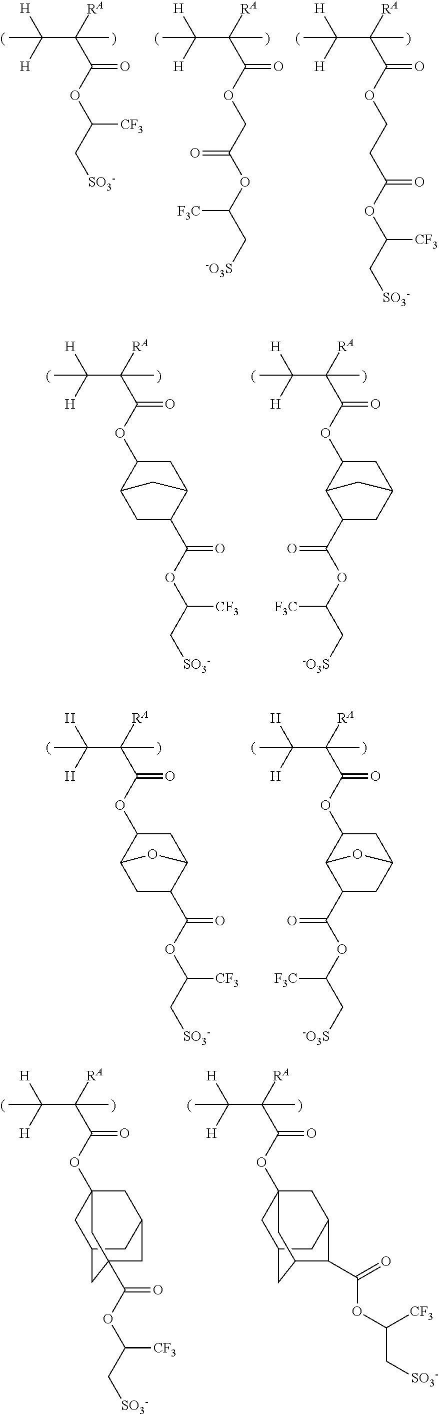

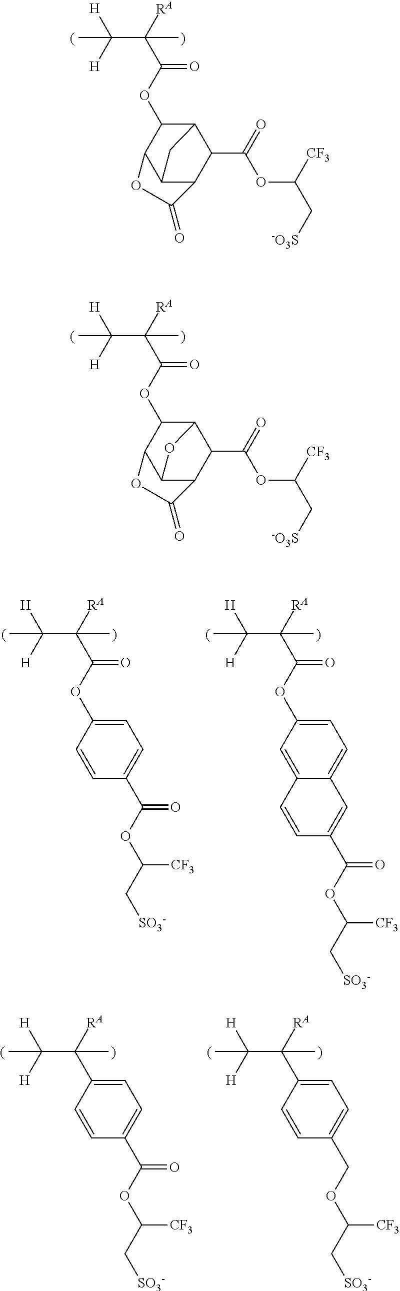

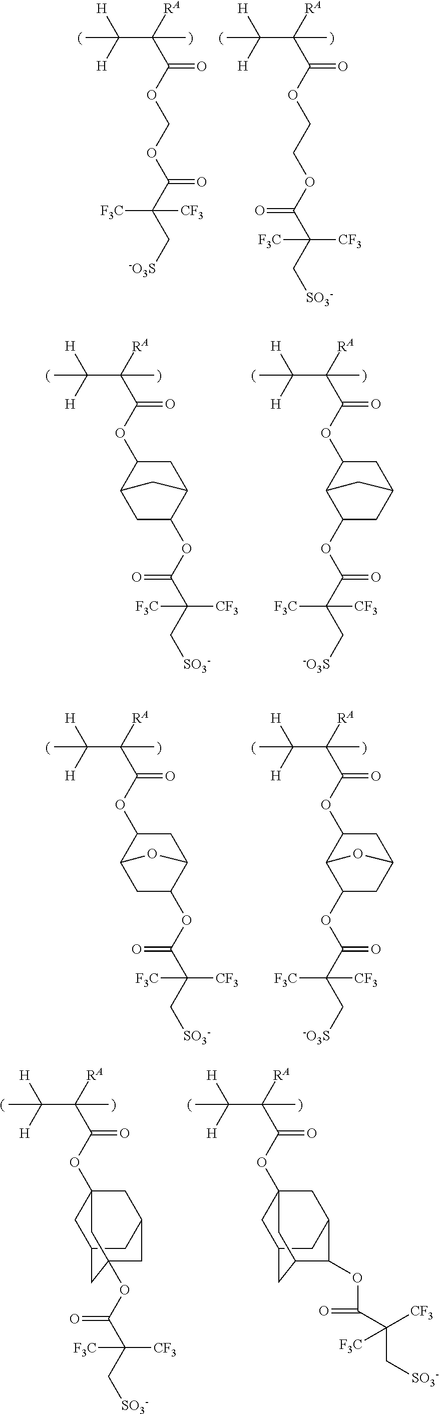

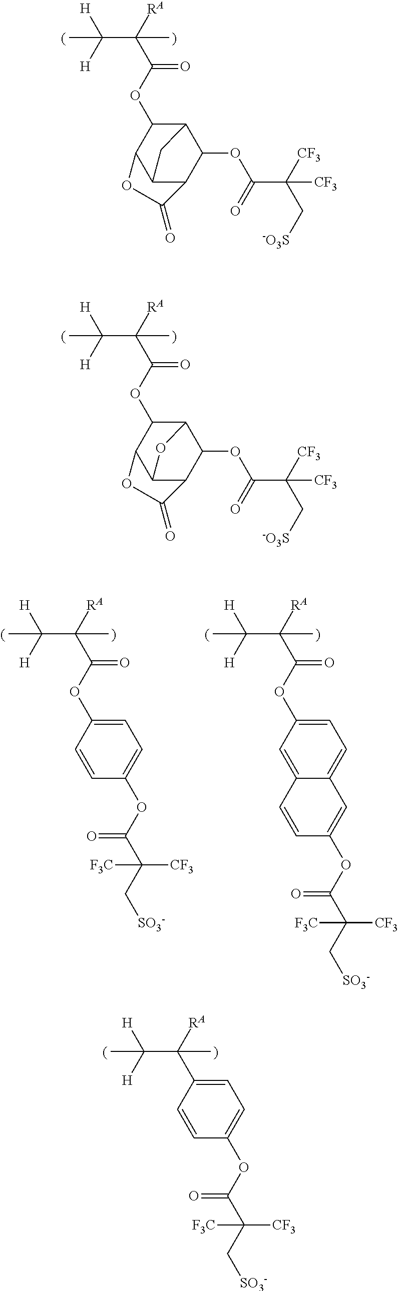

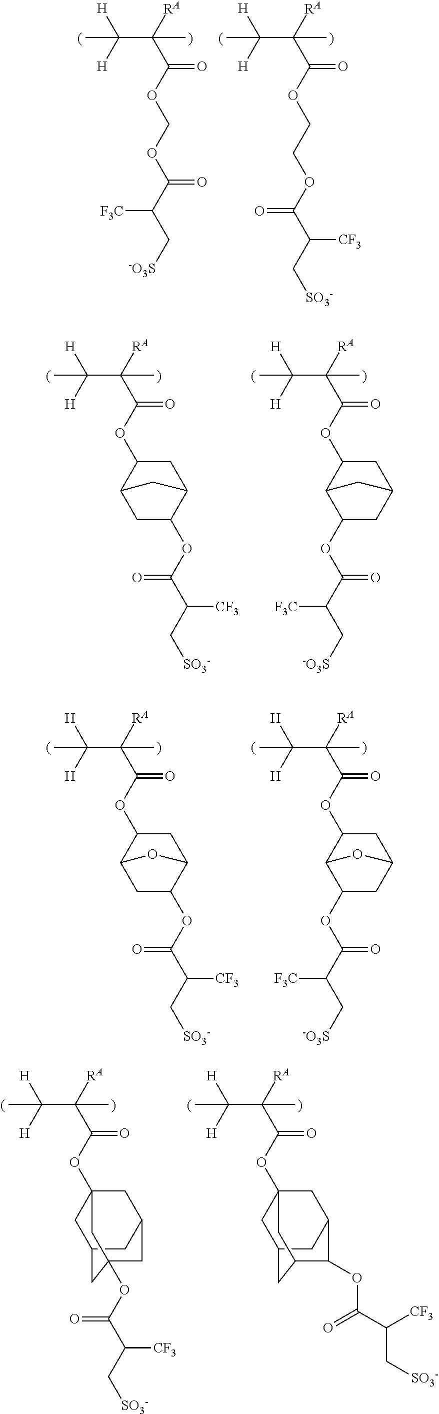

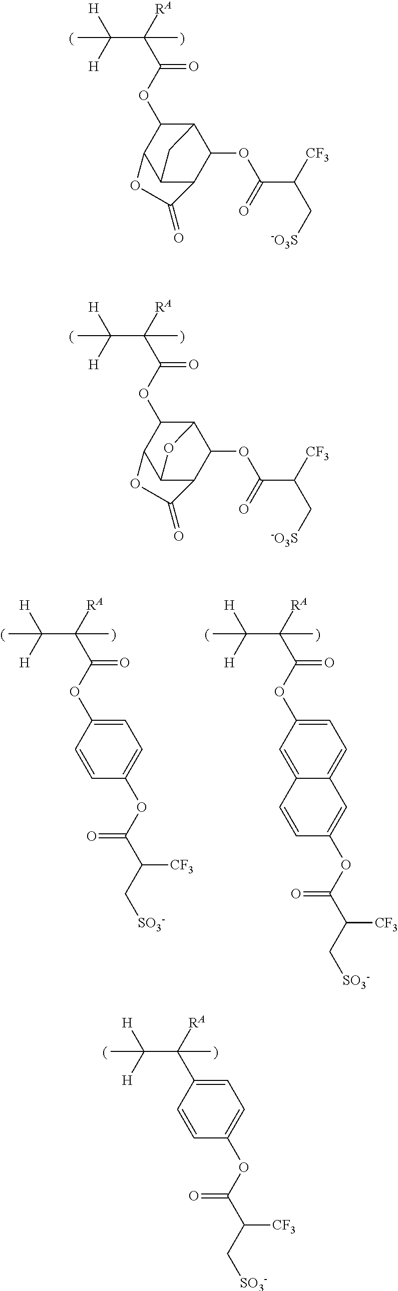

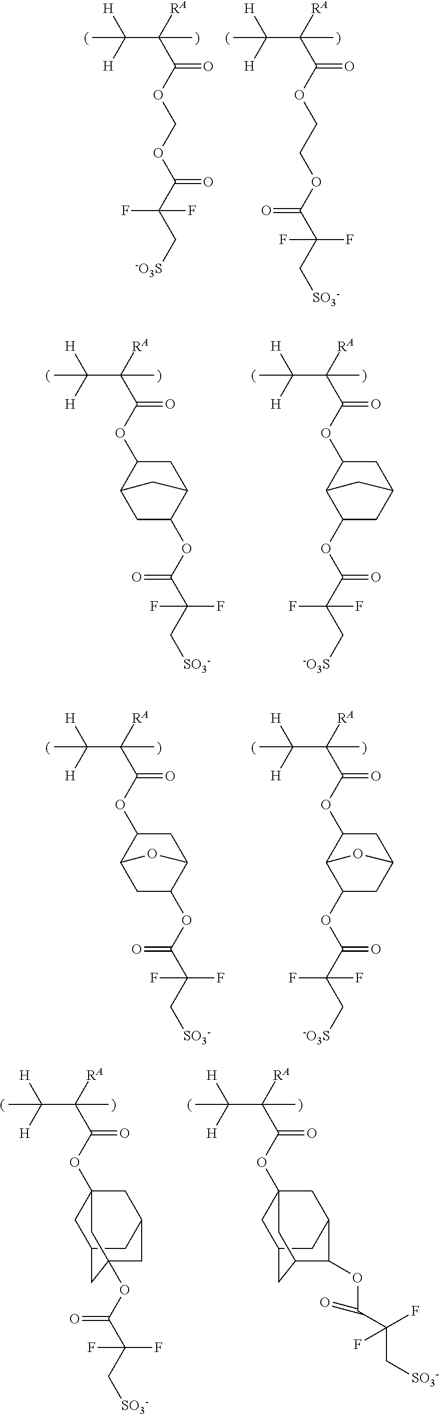

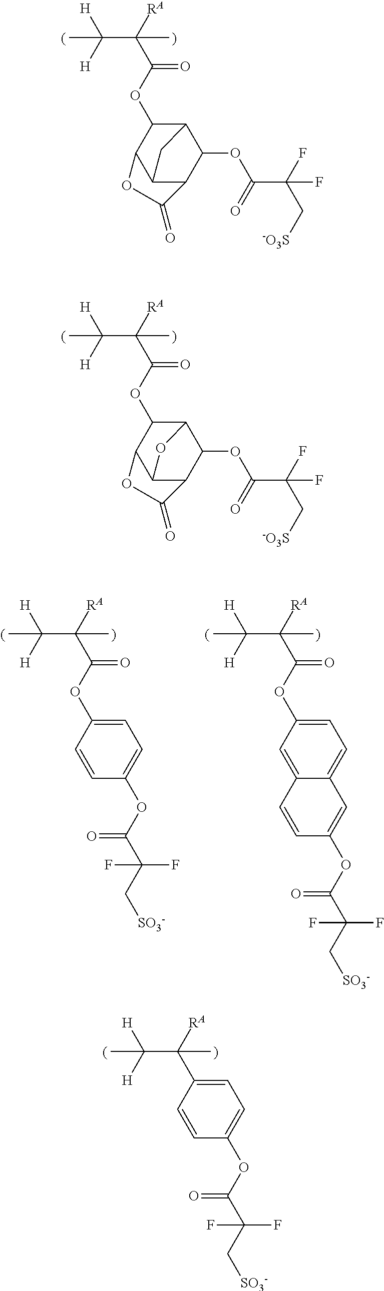

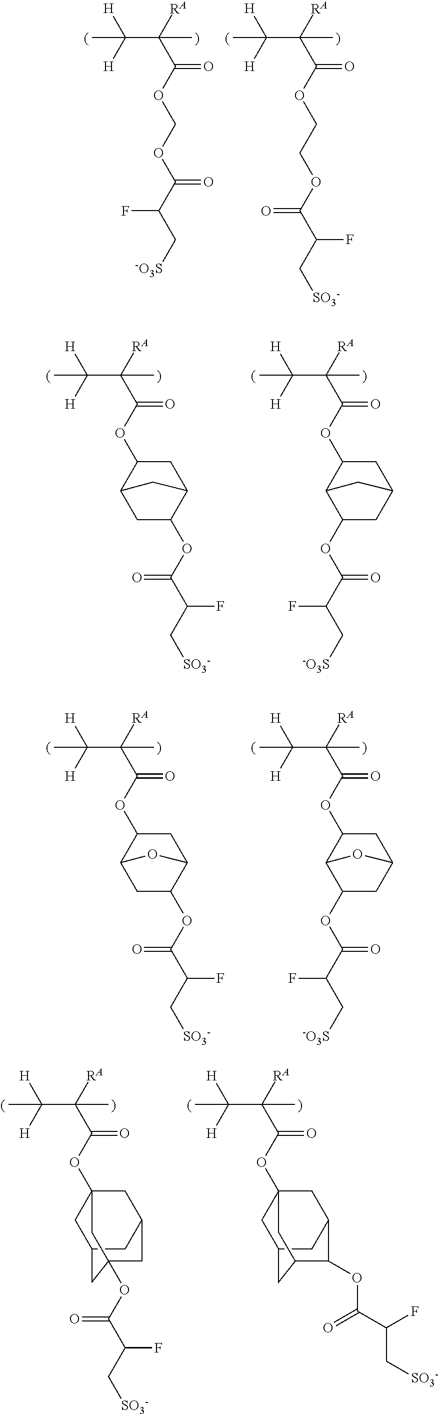

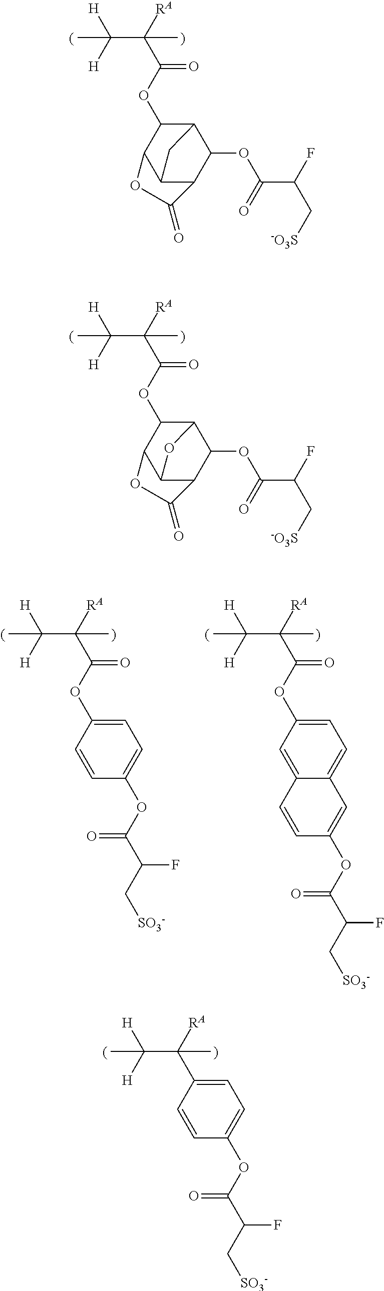

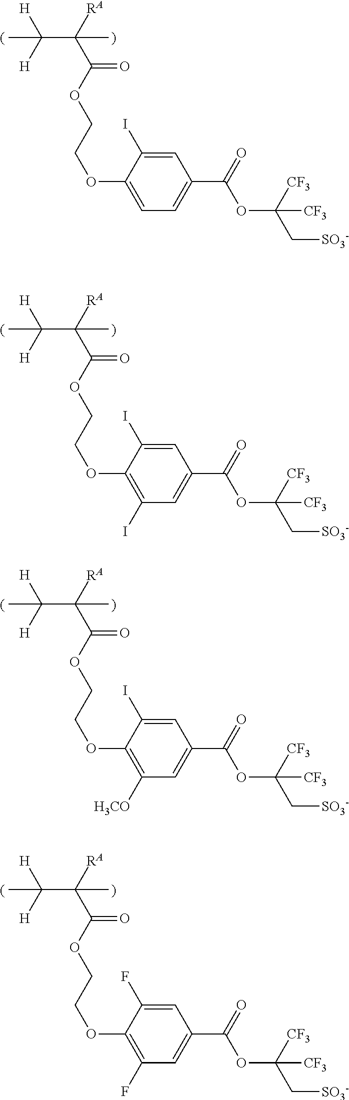

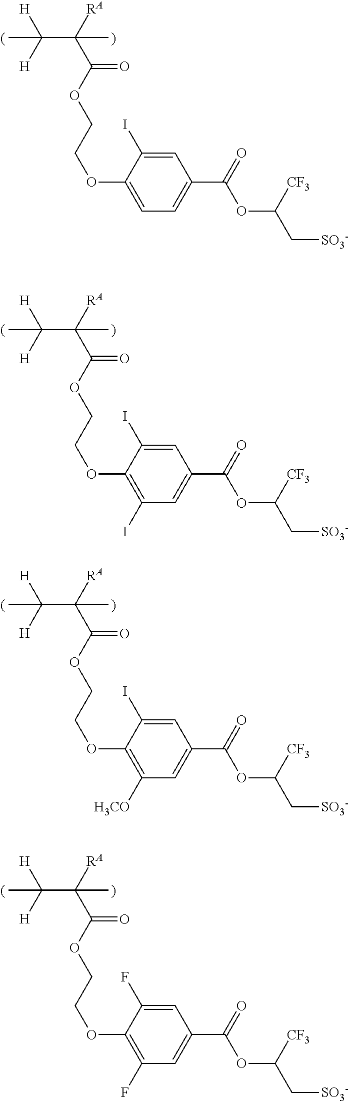

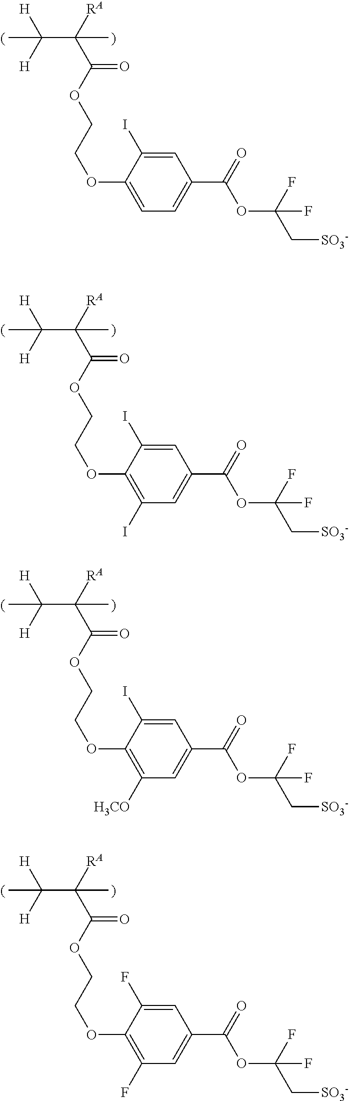

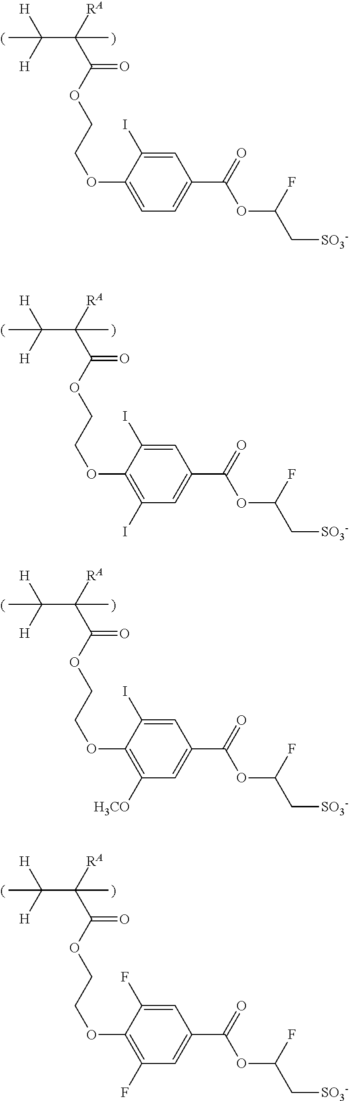

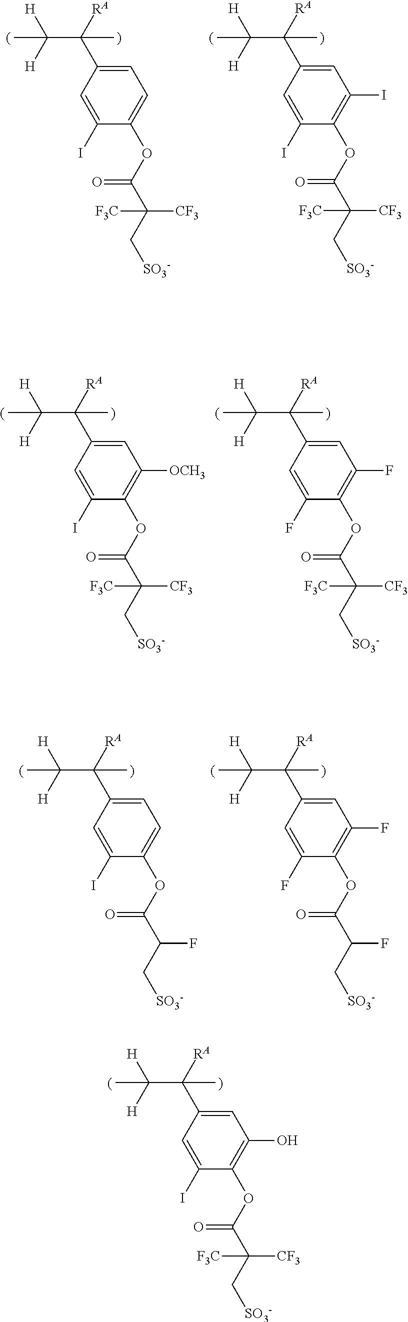

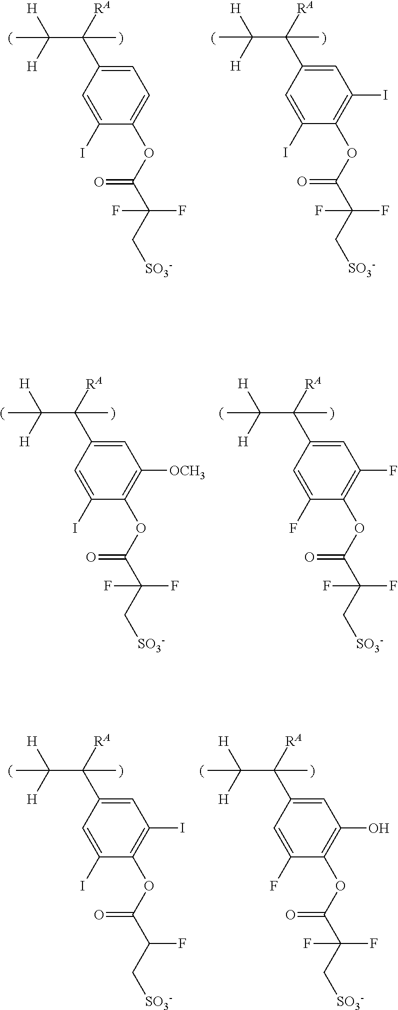

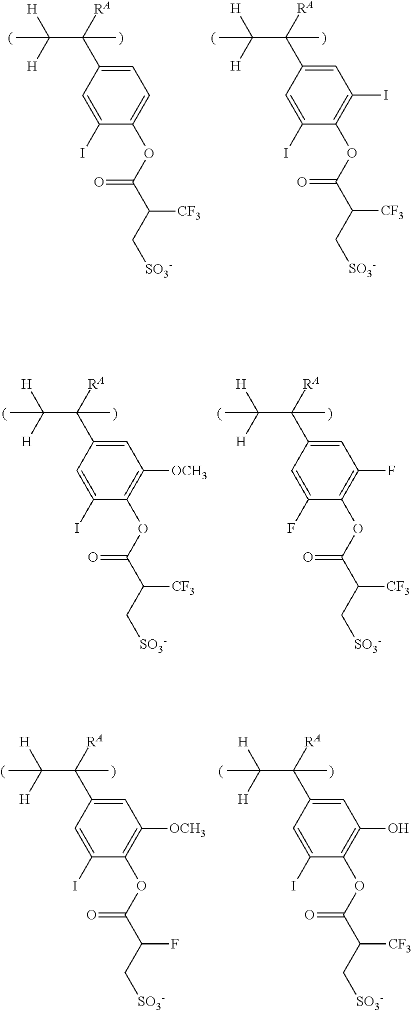

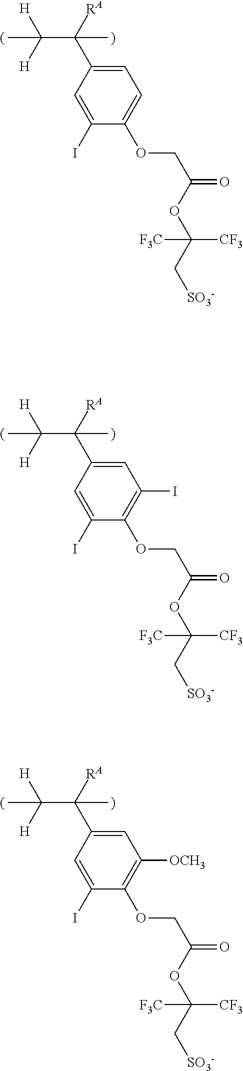

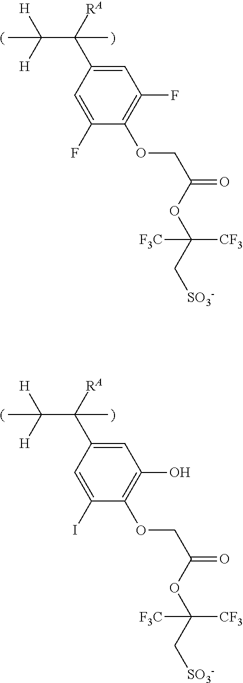

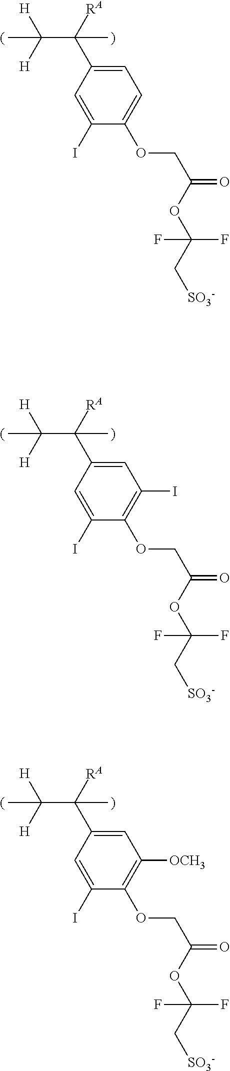

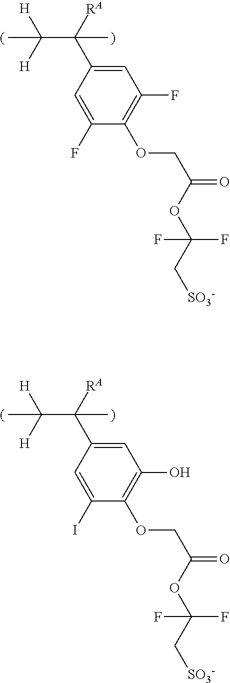

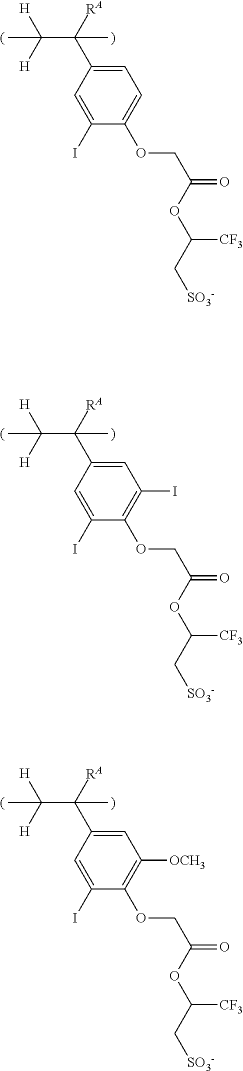

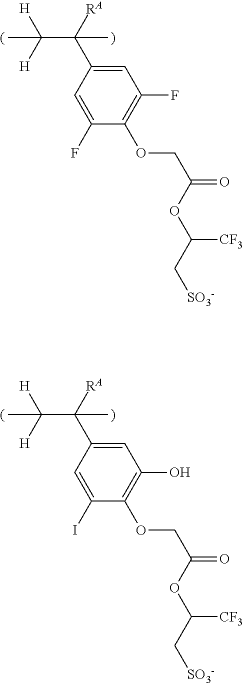

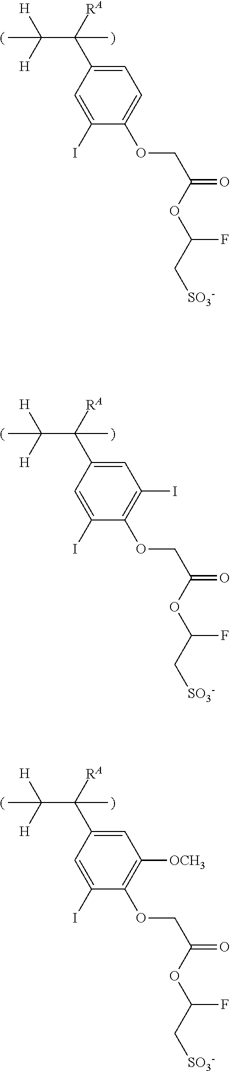

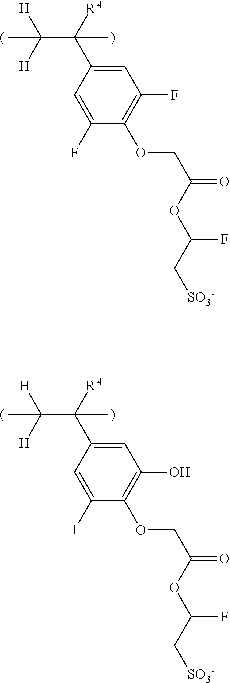

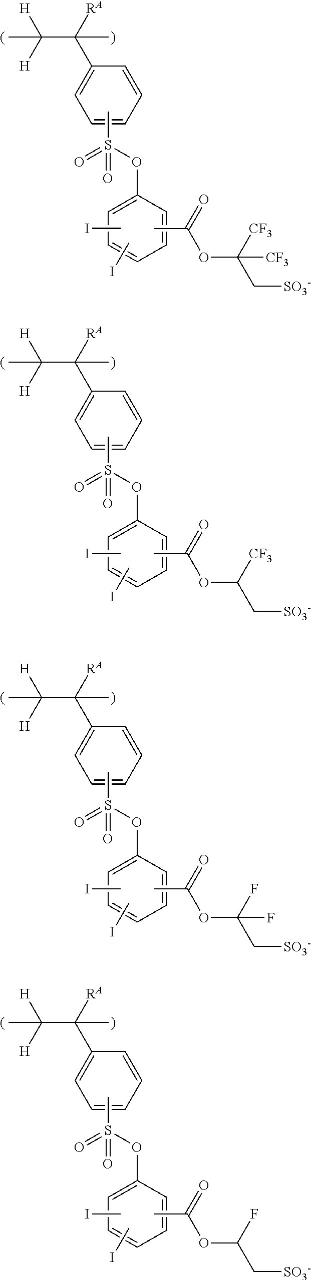

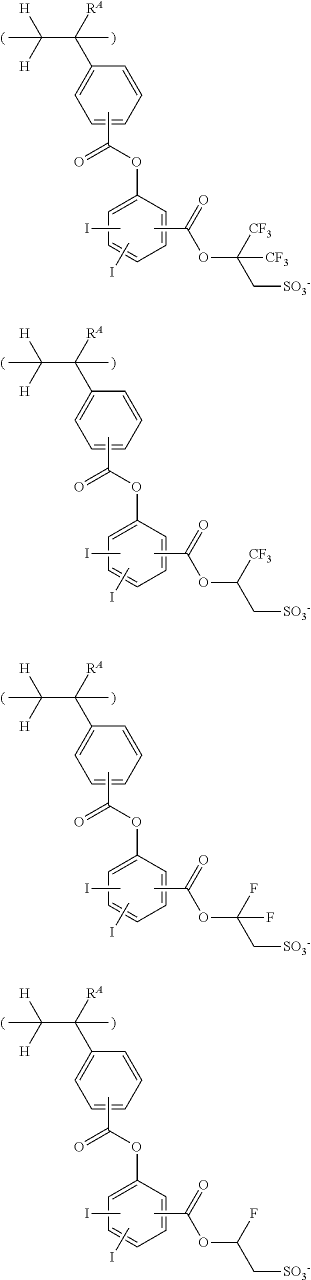

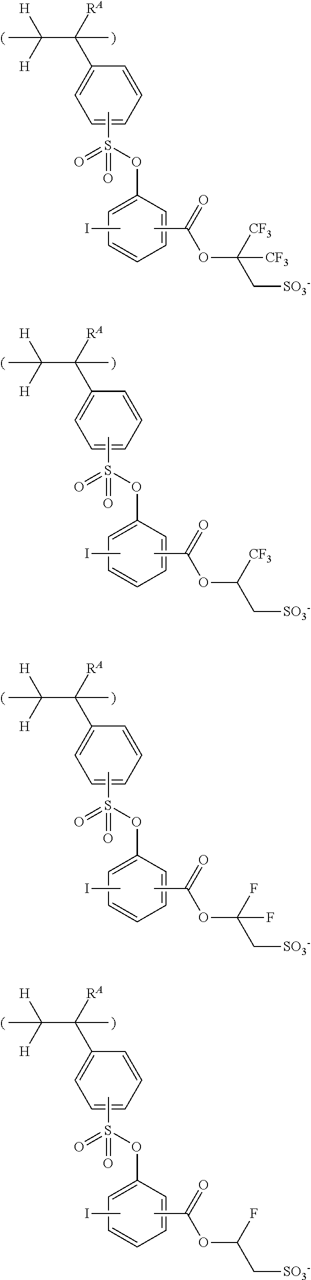

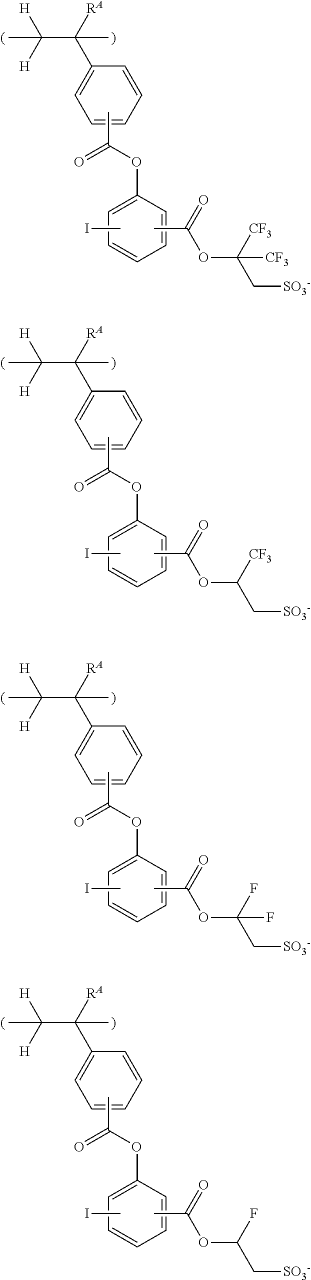

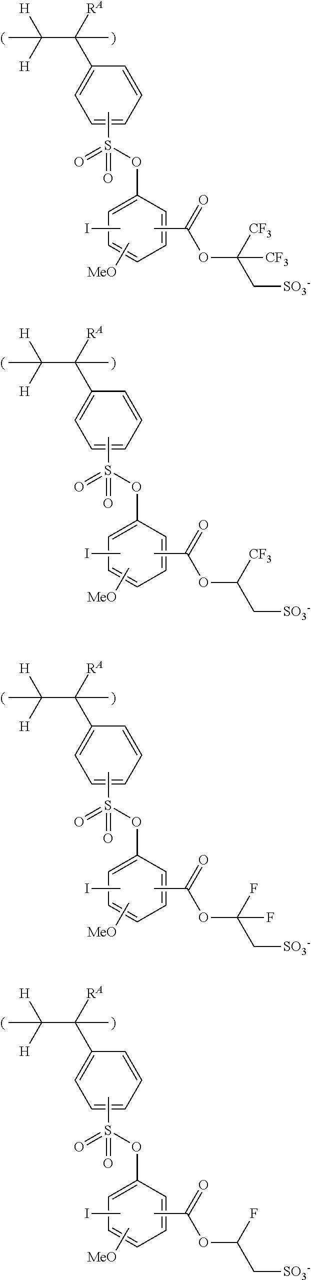

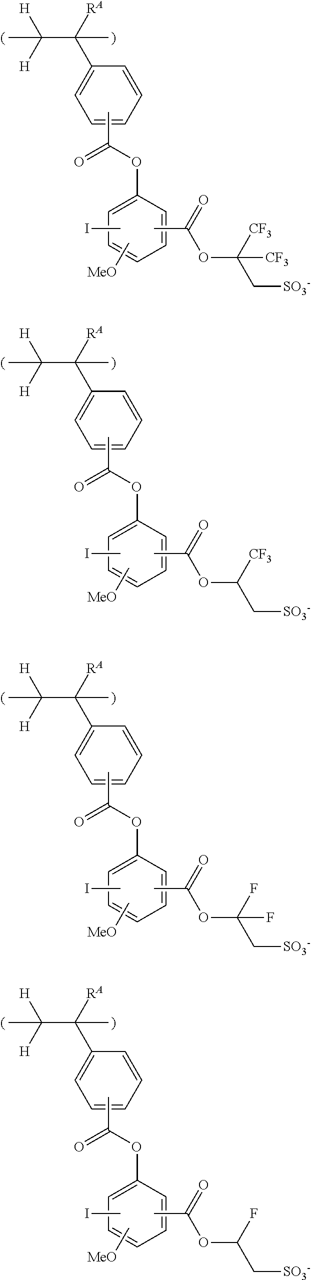

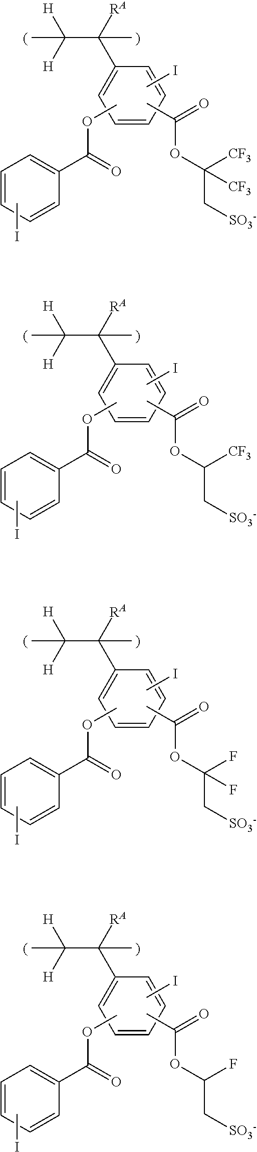

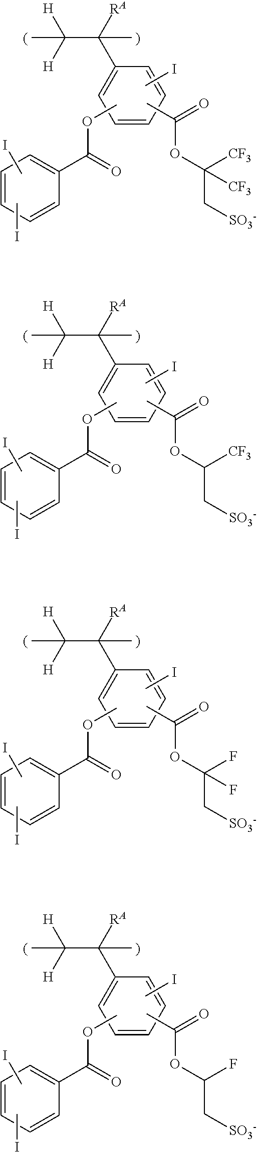

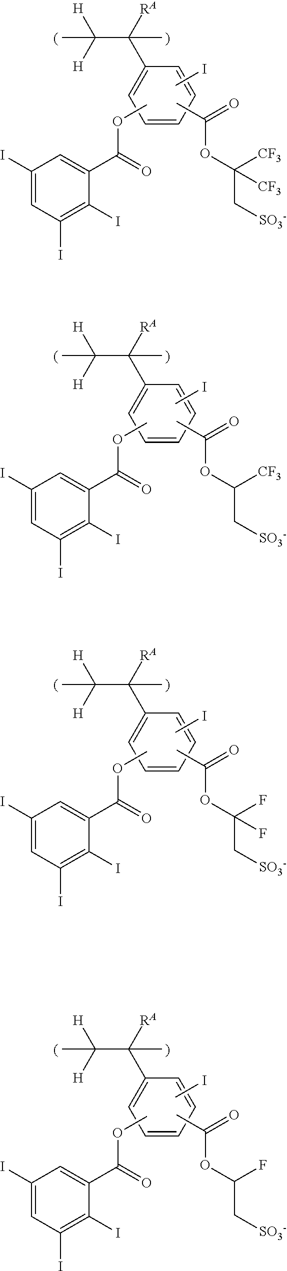

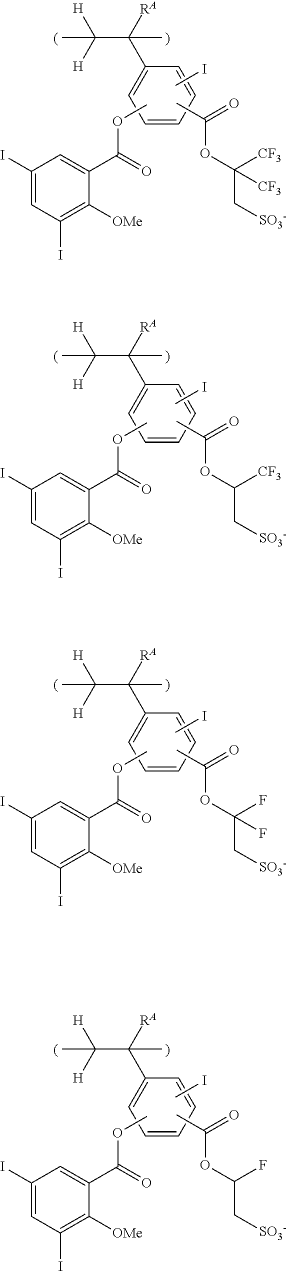

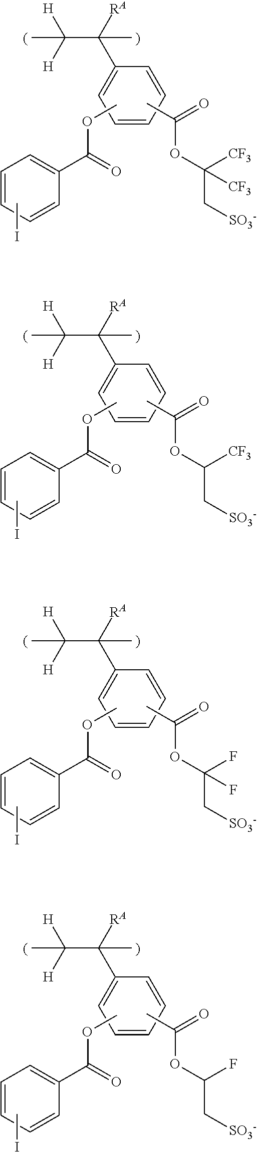

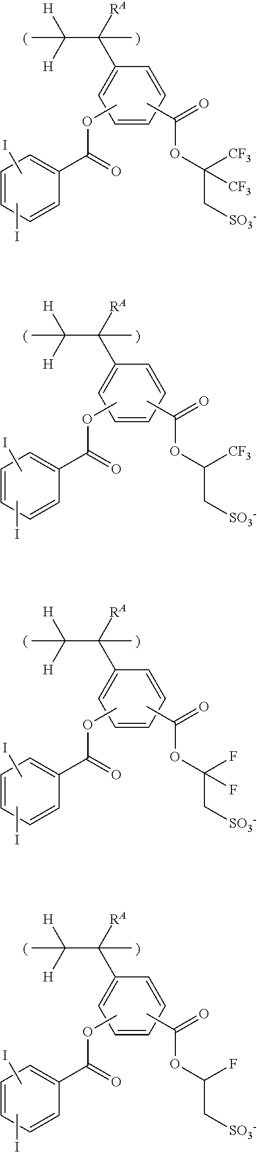

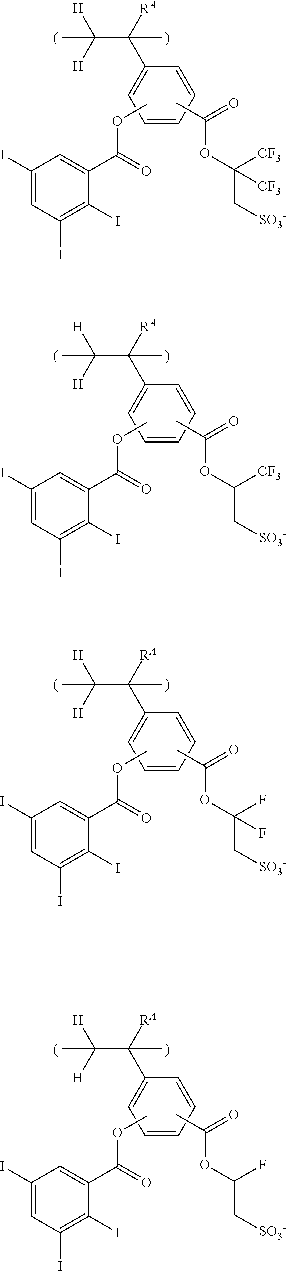

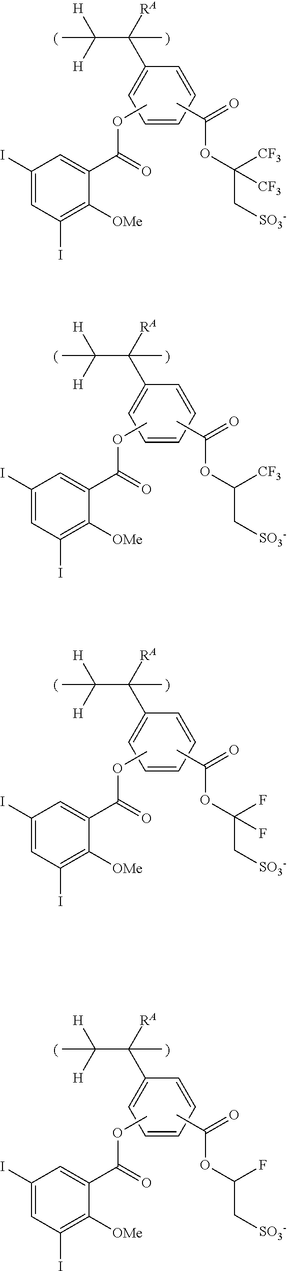

Specific examples of the anion of the repeat unit (c4) are shown below, but not limited thereto. In the following formulae. R^{A} is as defined above.

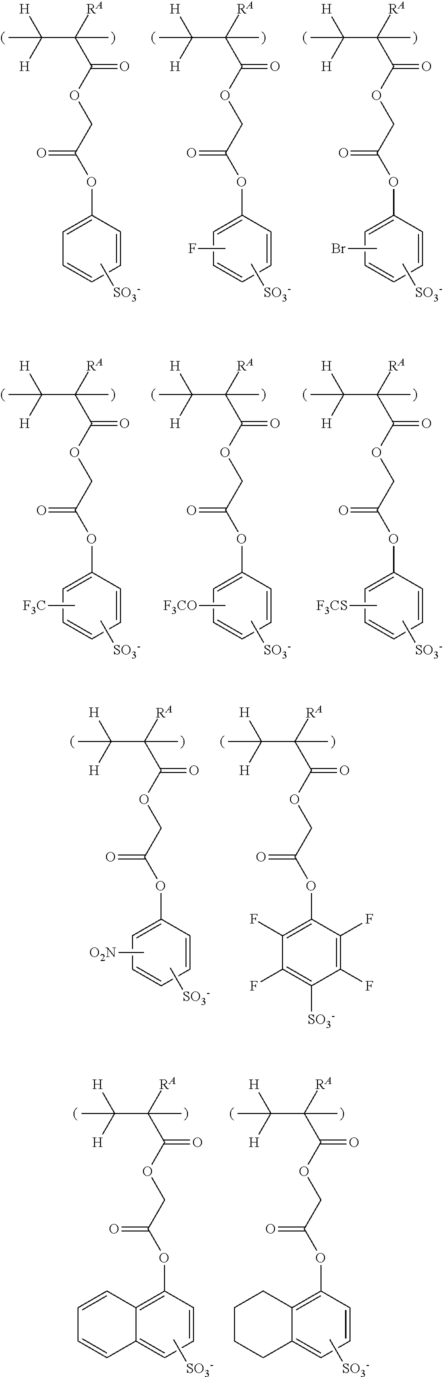

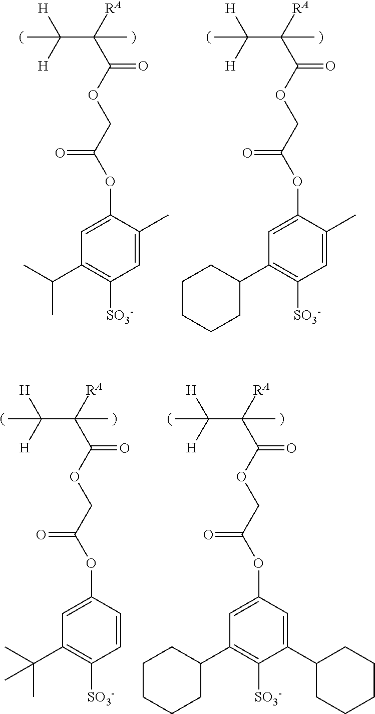

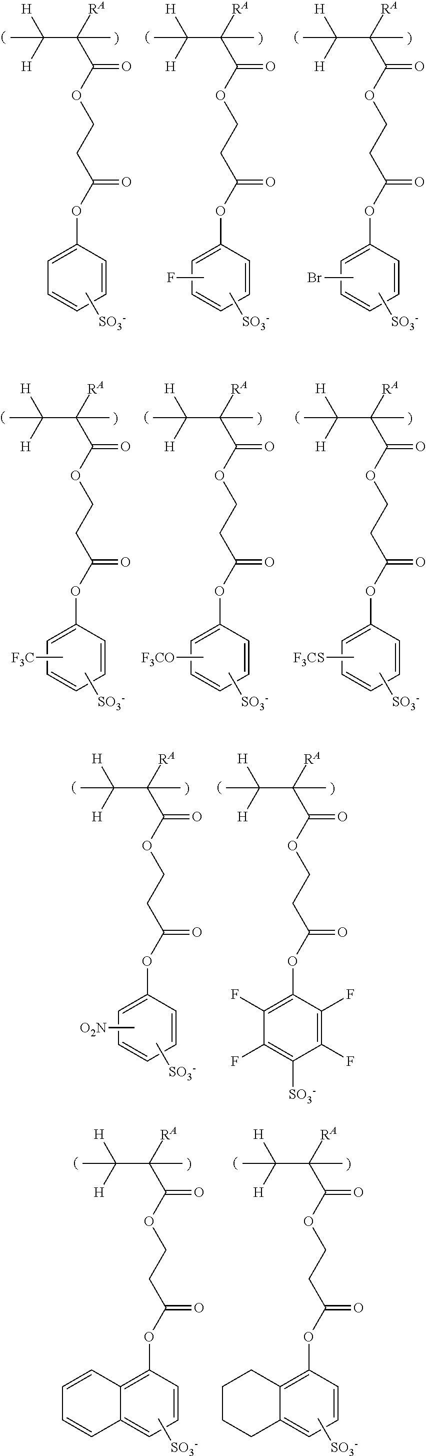

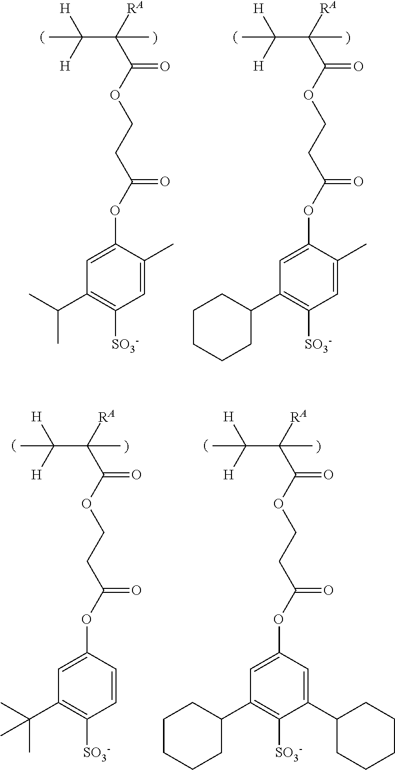

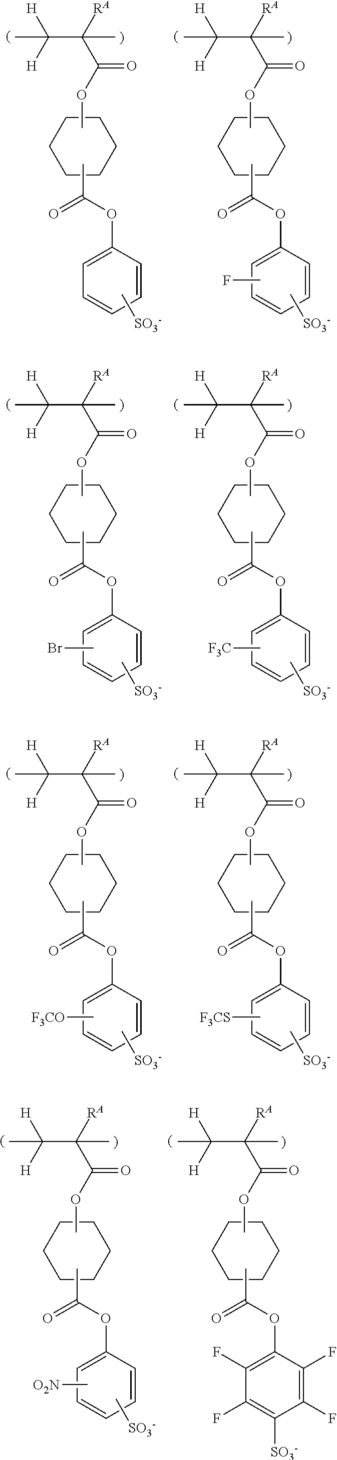

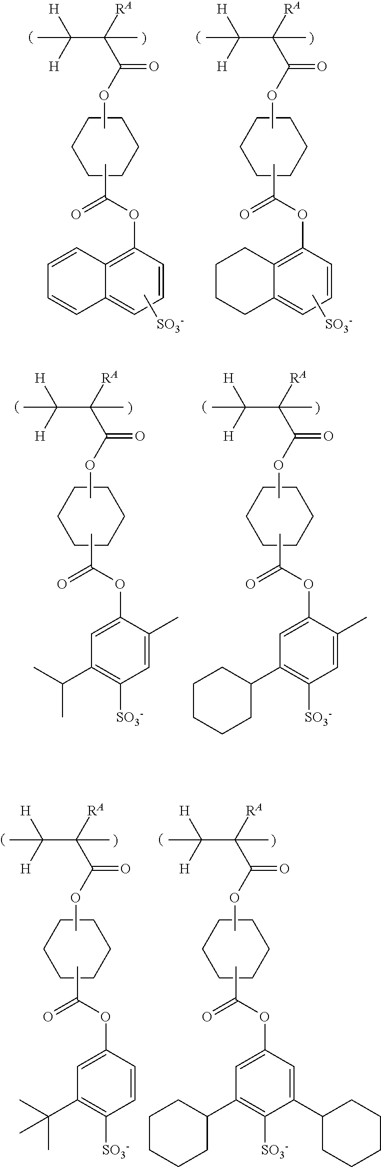

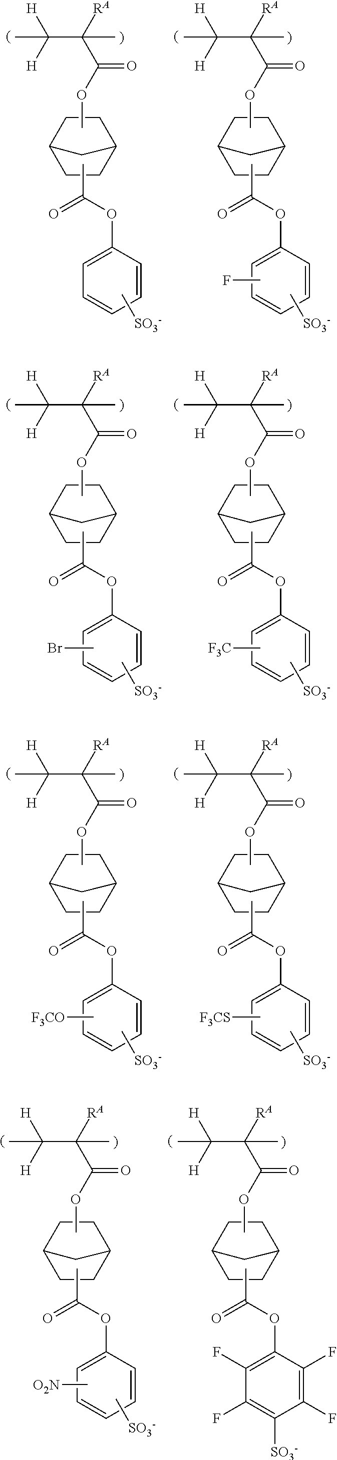

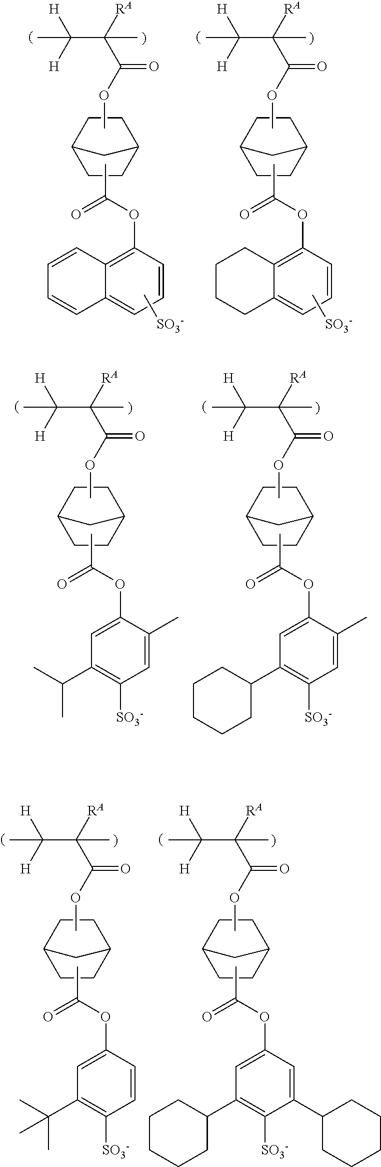

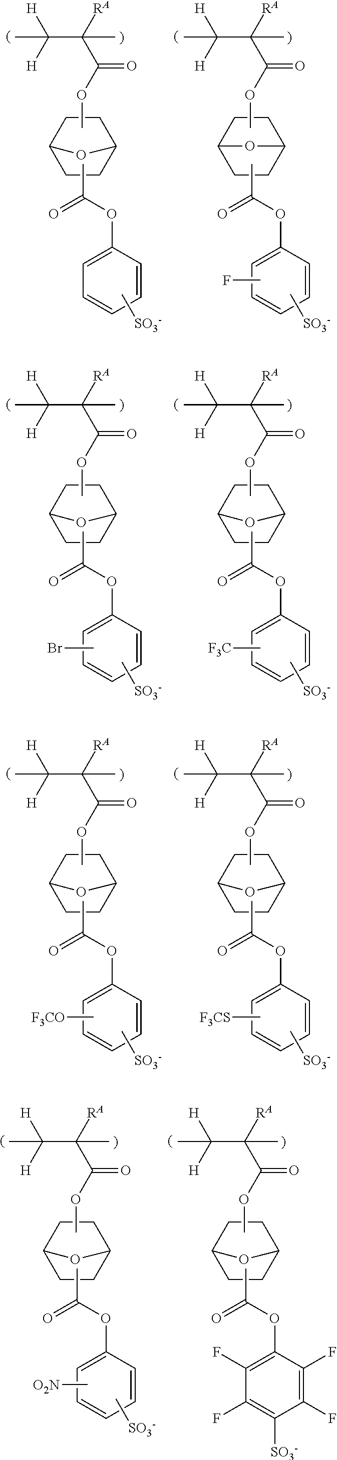

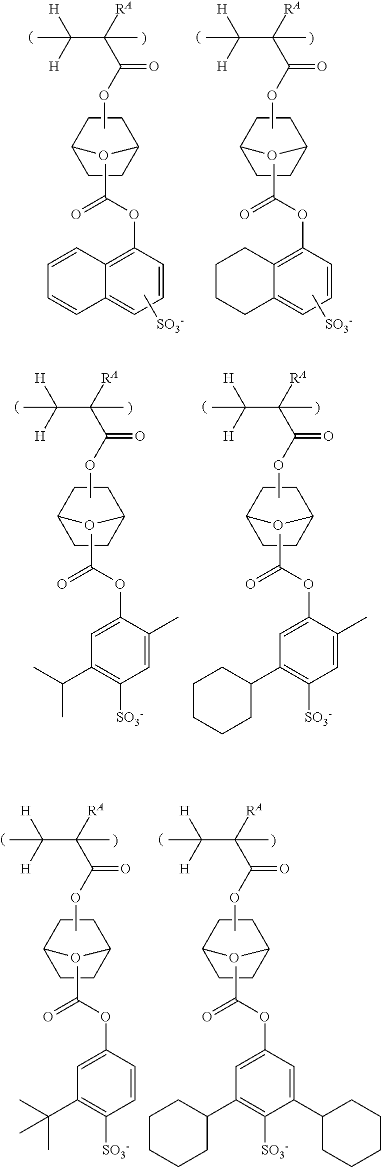

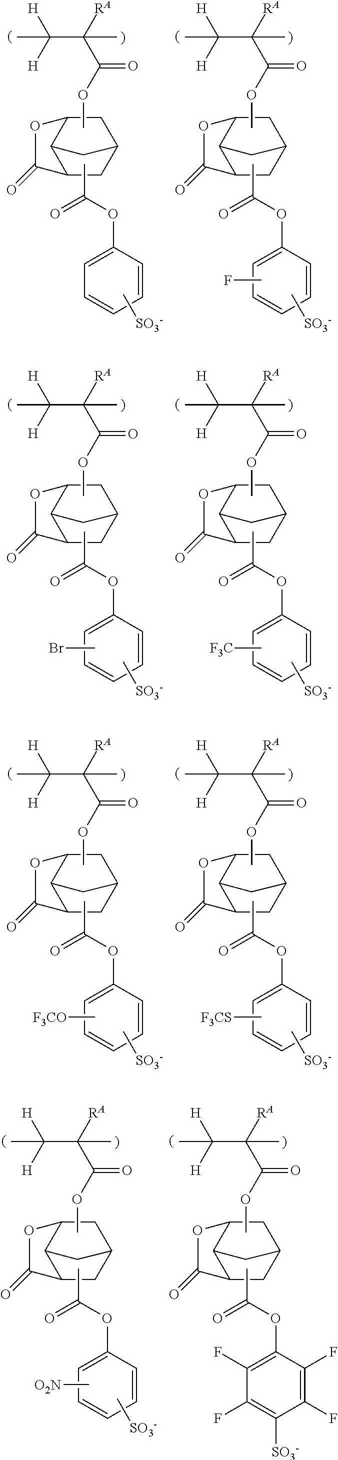

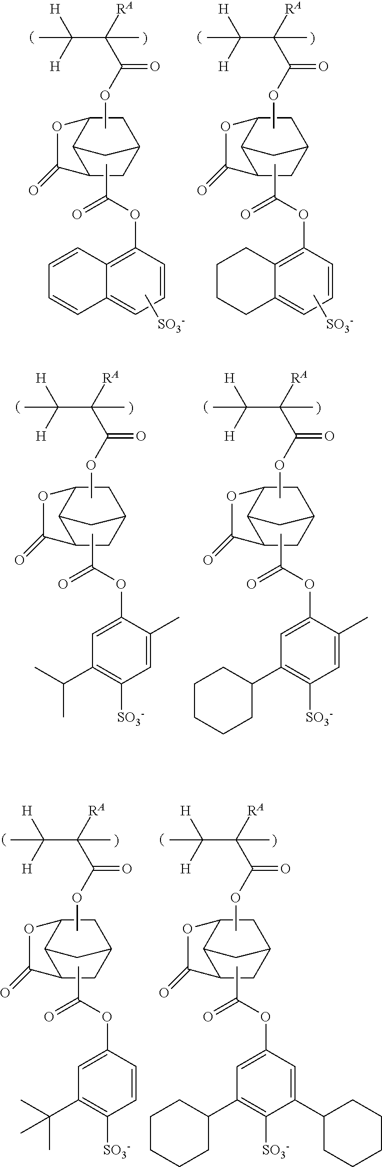

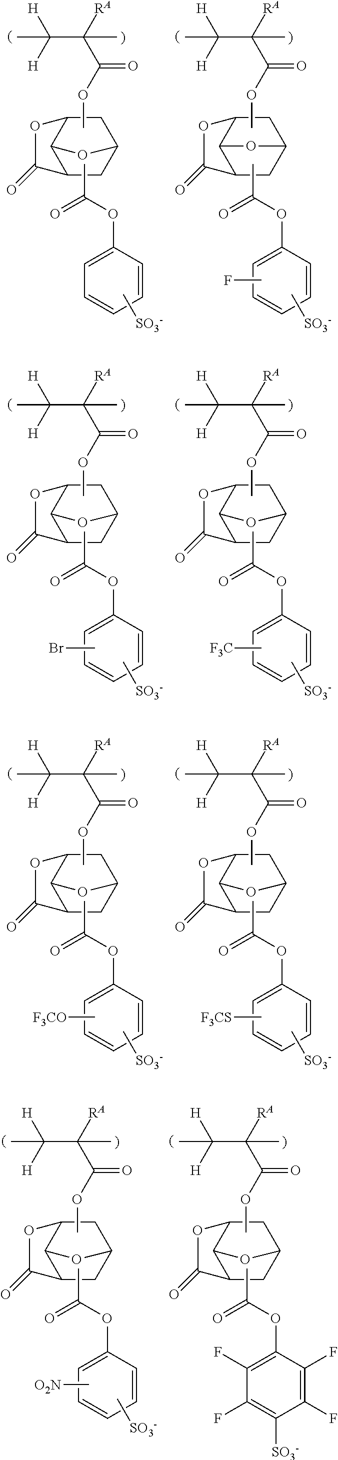

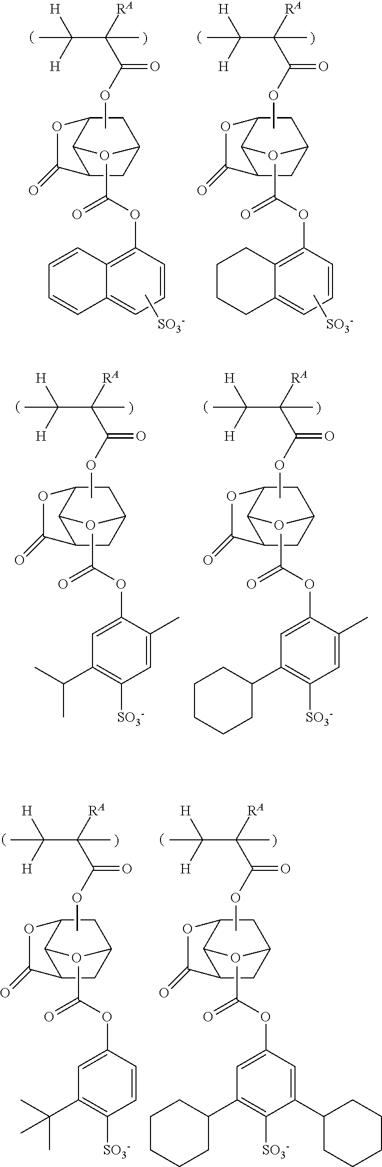

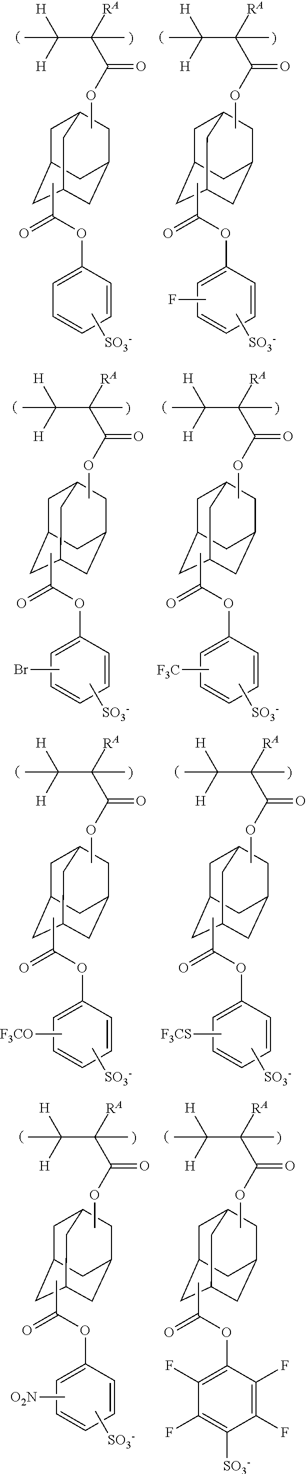

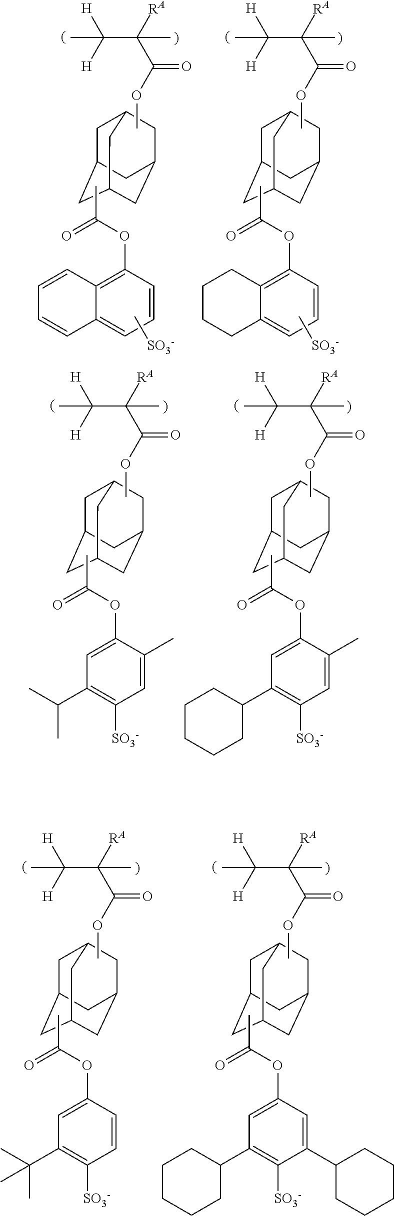

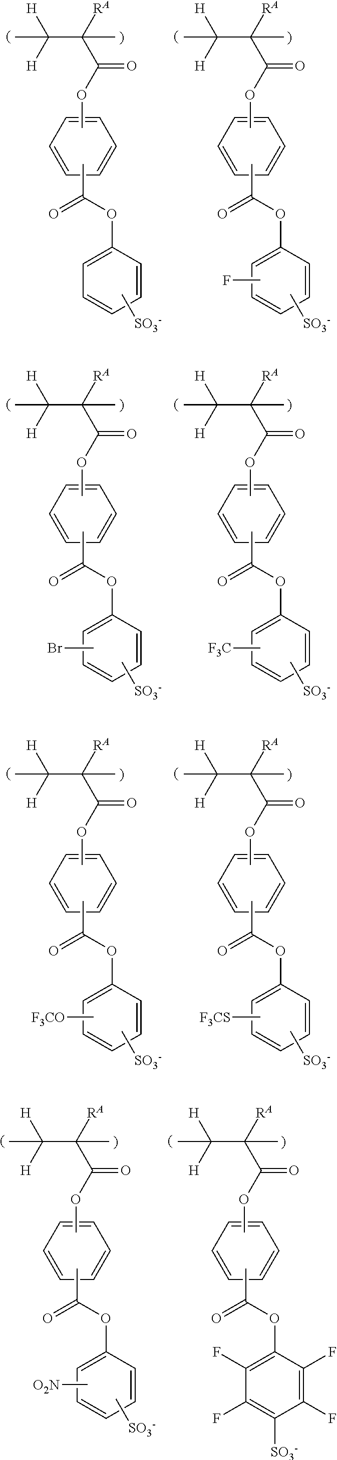

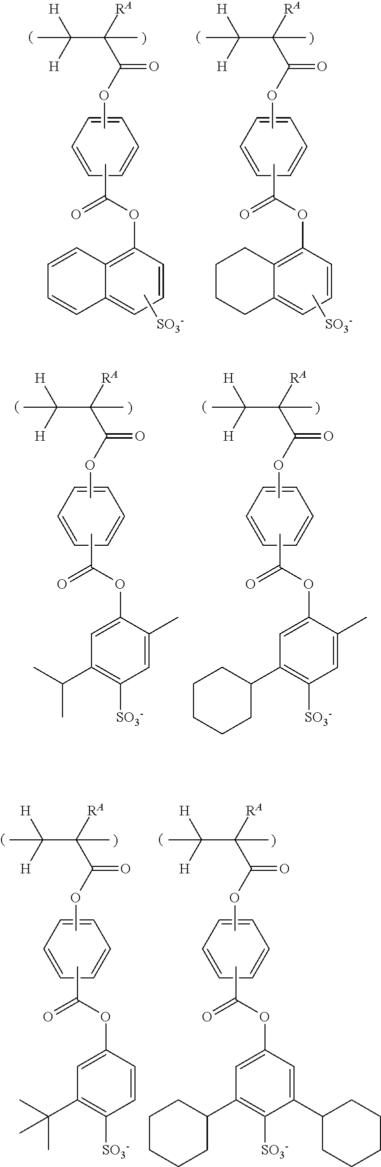

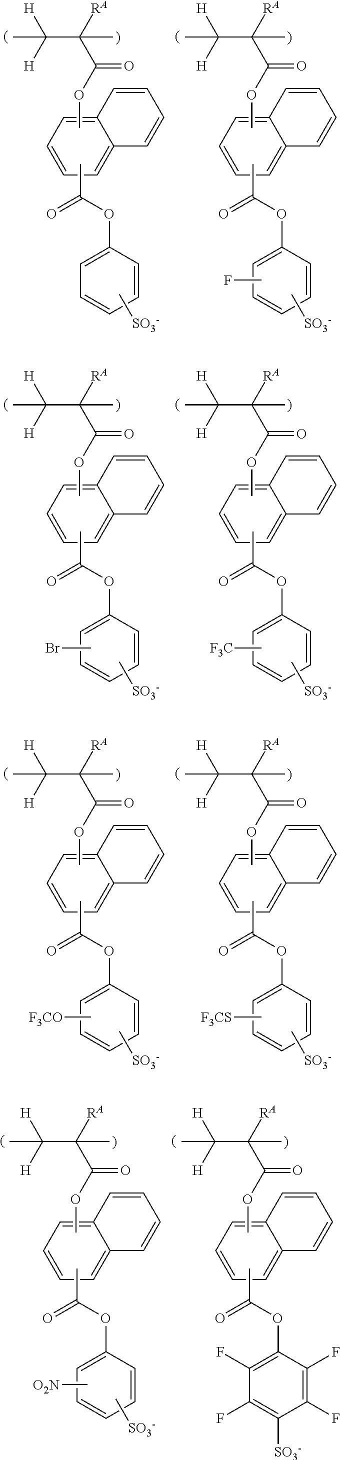

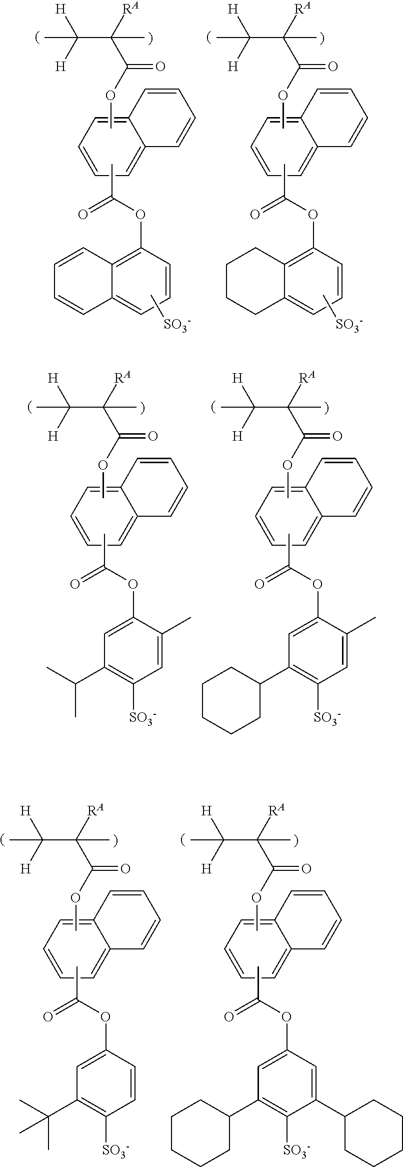

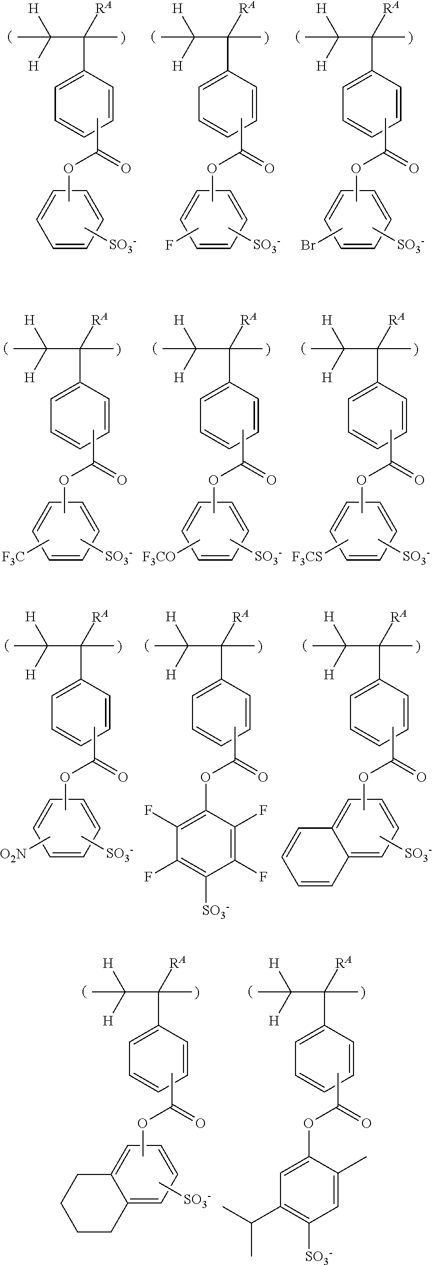

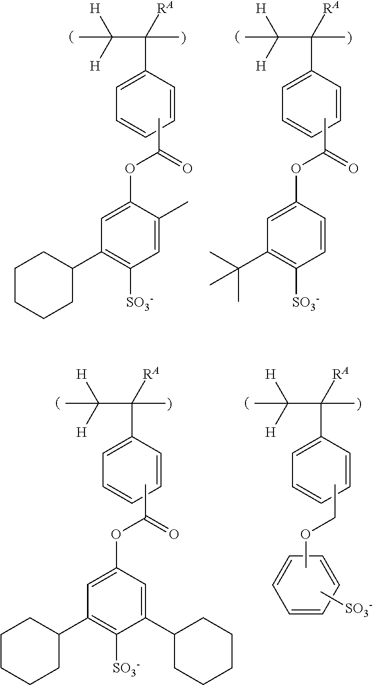

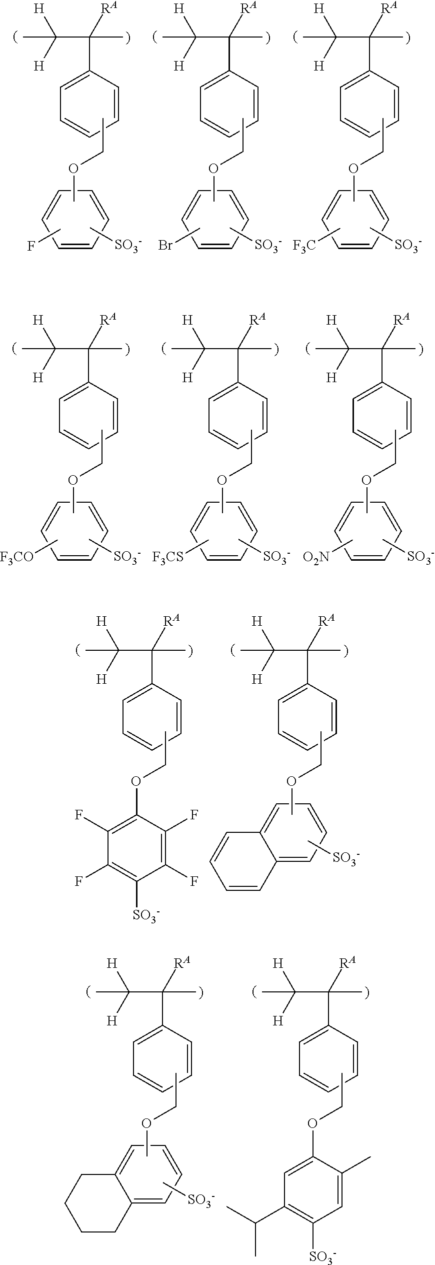

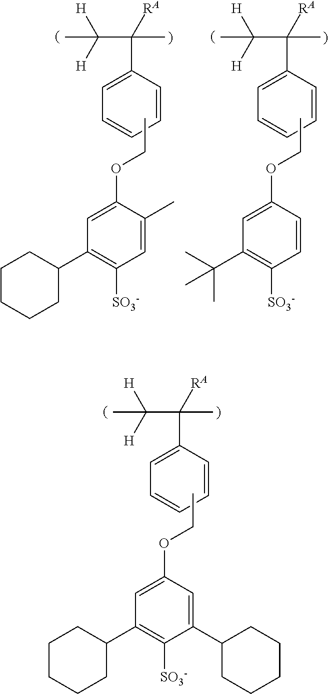

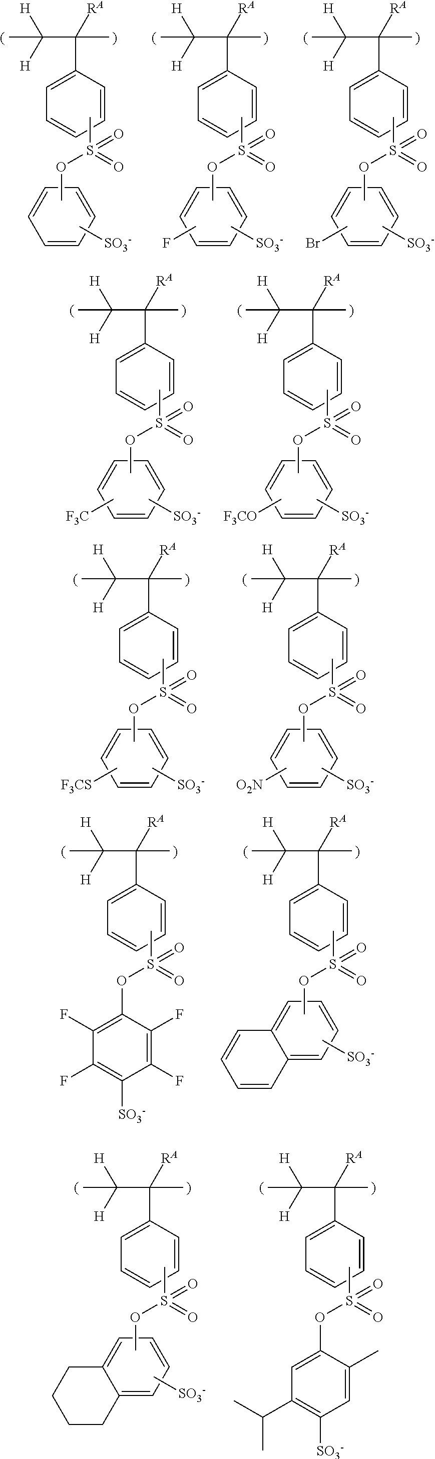

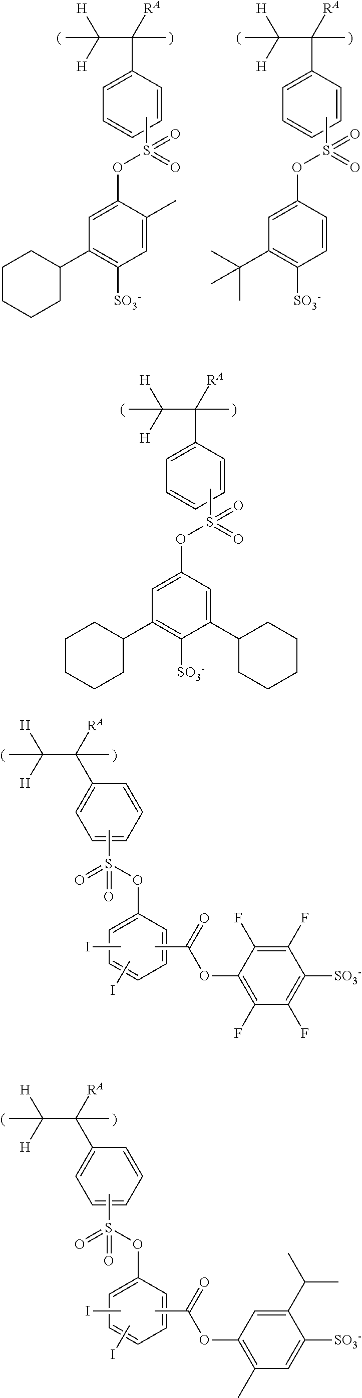

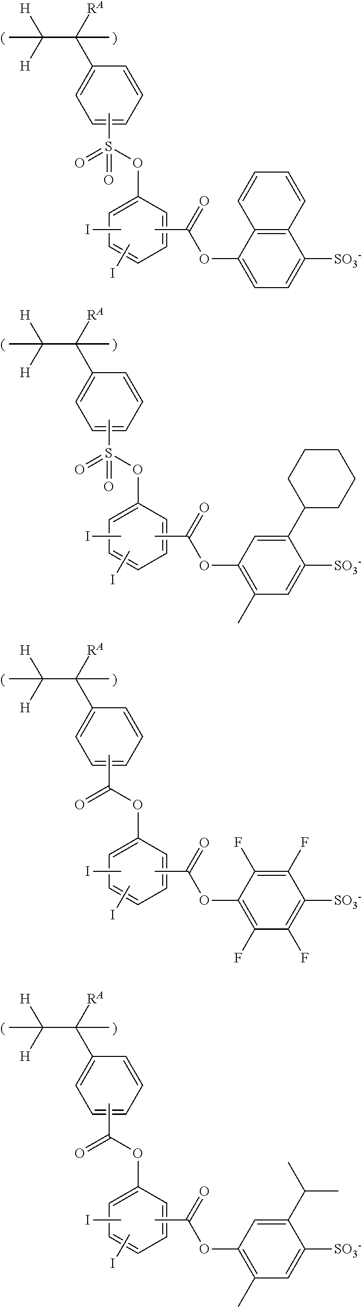

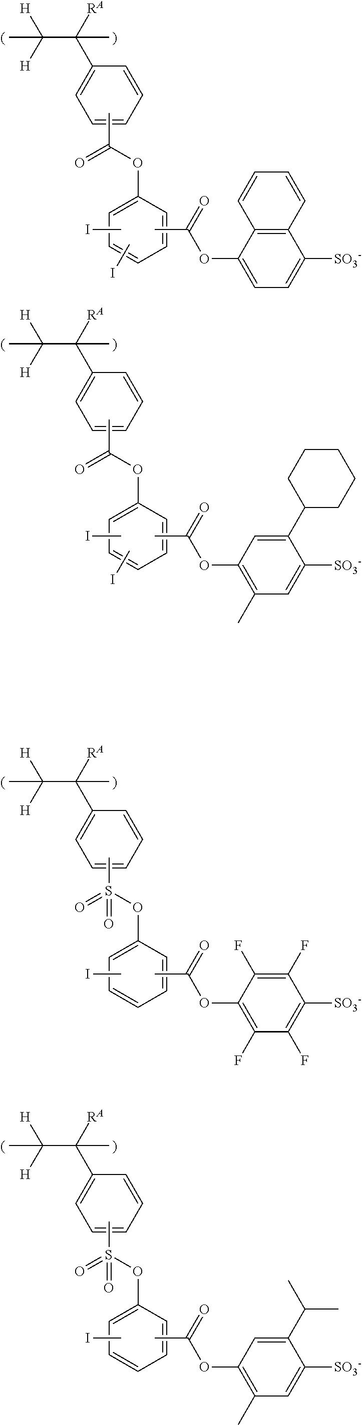

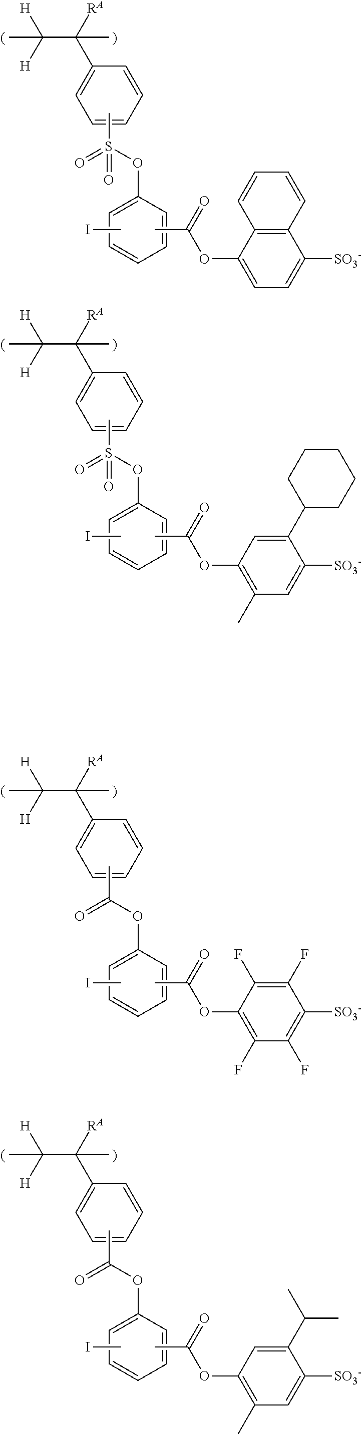

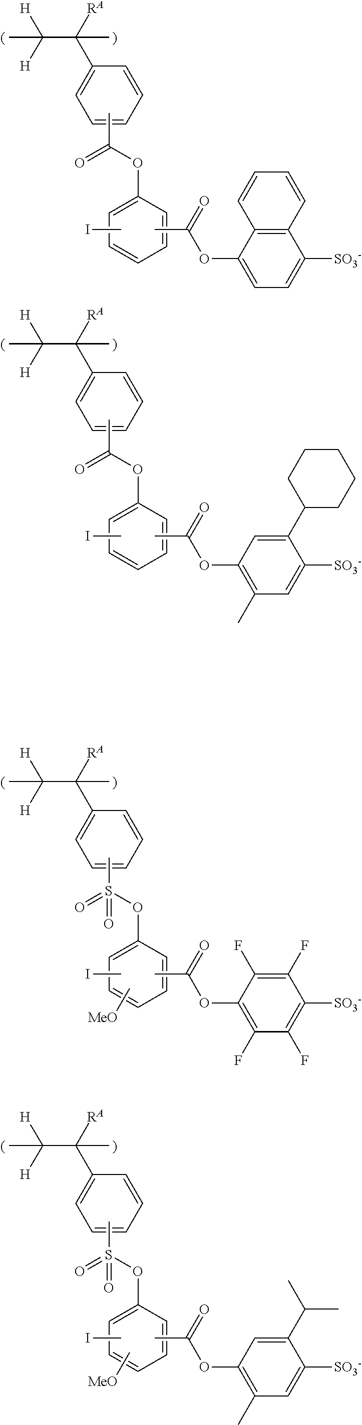

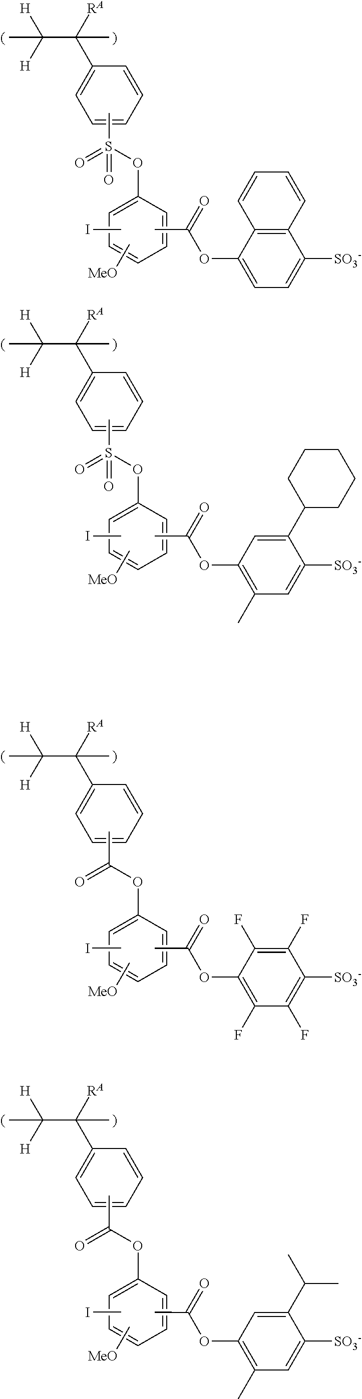

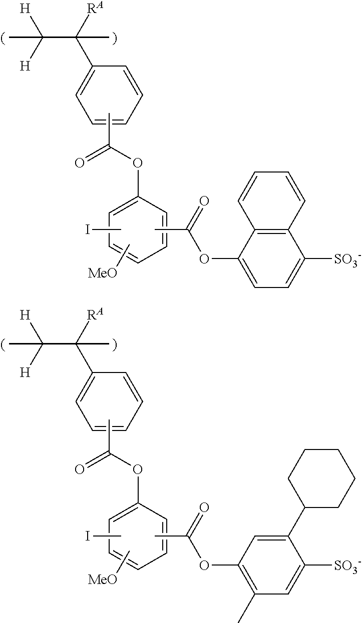

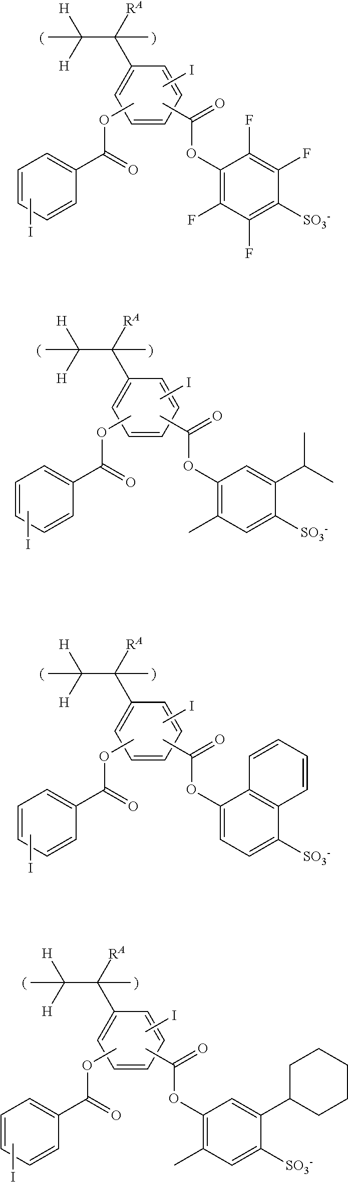

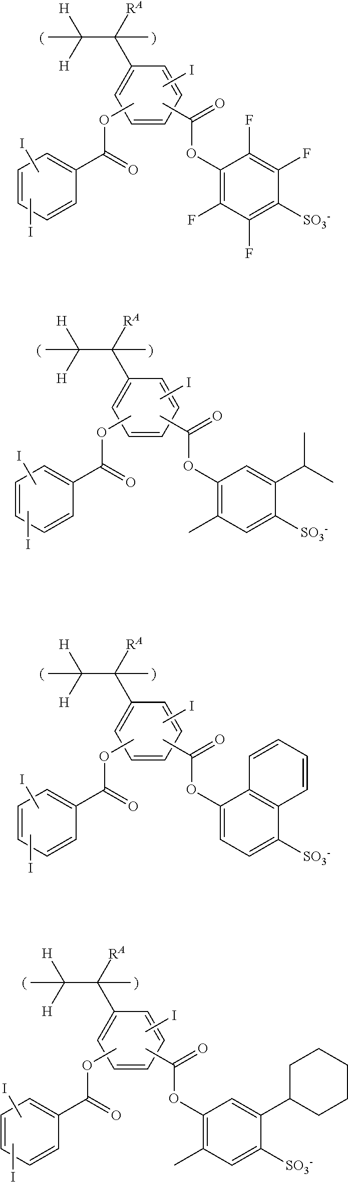

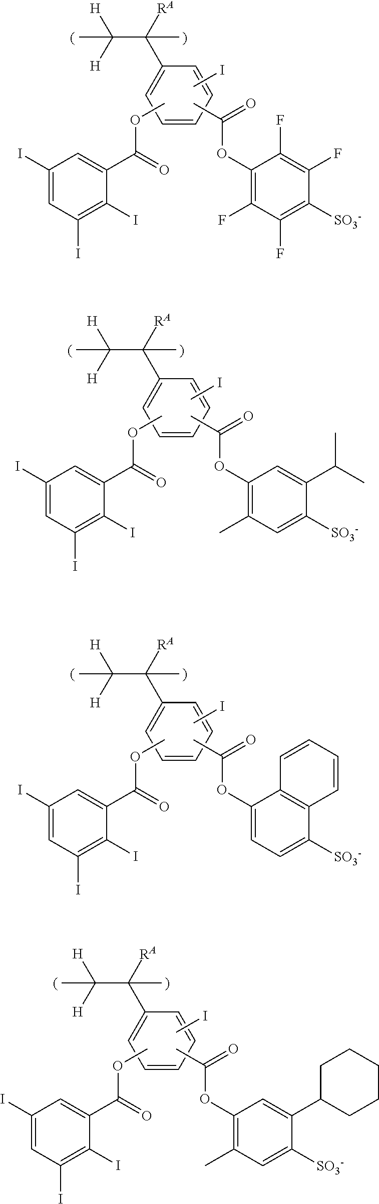

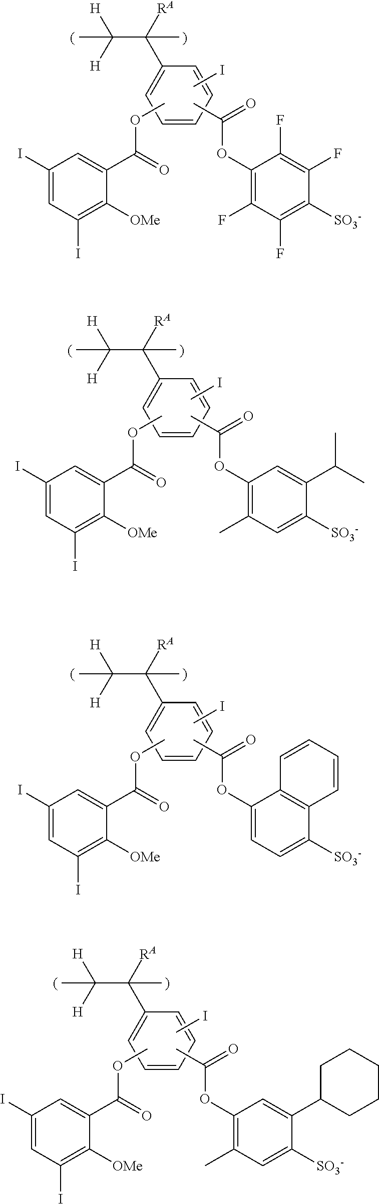

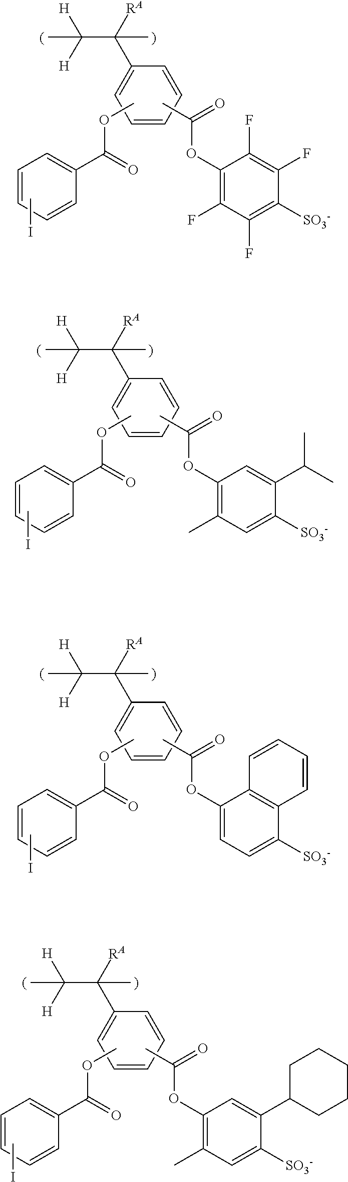

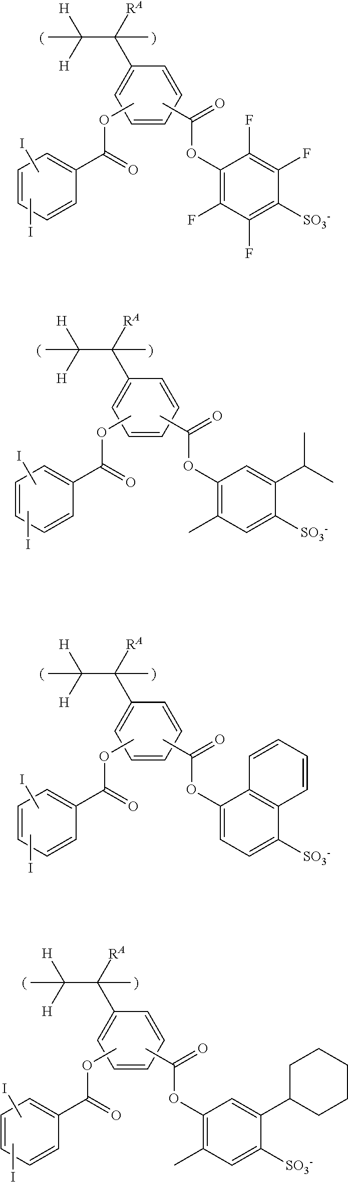

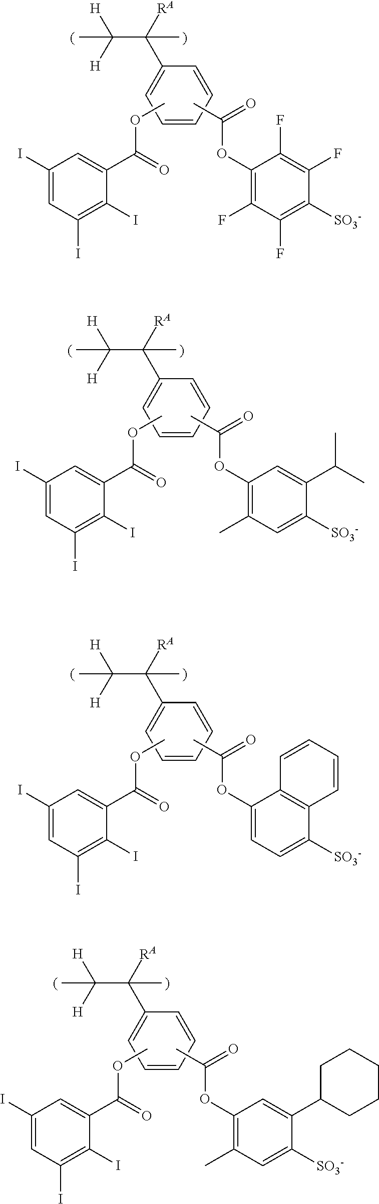

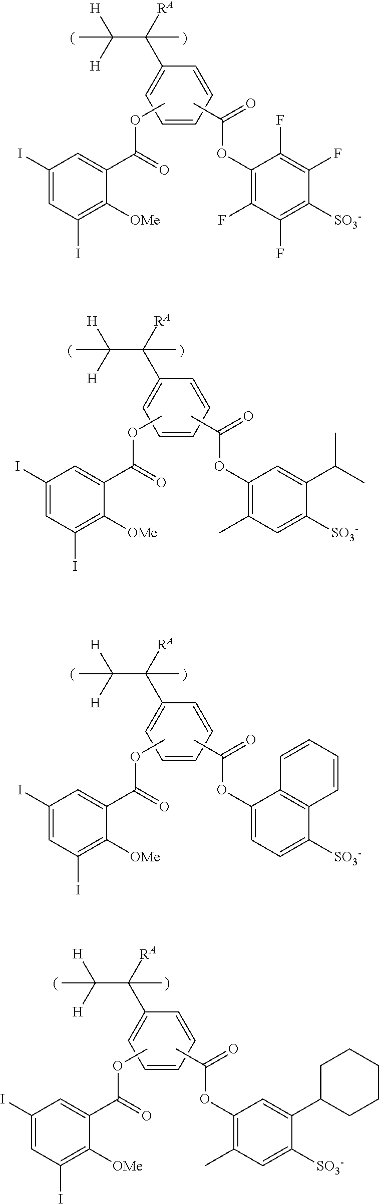

Specific examples of the anion of the repeat unit (c5) are shown below, but not limited thereto. In the following formulae, R^{A} is as defined above.

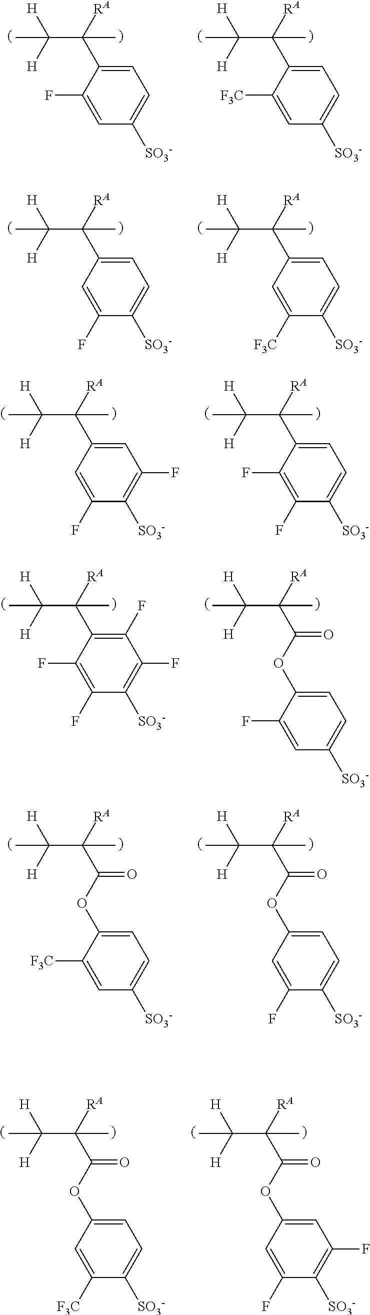

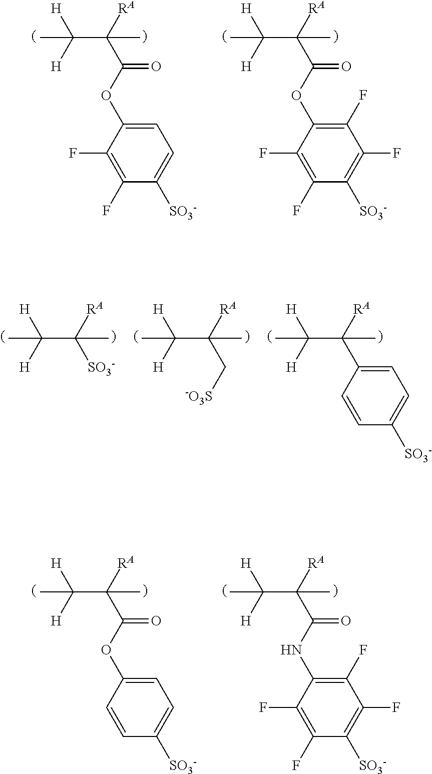

In the formulae (c2) to (c5), A⁺ is an onium cation. Examples of the onium cation include a sulfonium cation, an iodonium cation, and an ammonium cation, and a sulfonium cation and an iodonium cation are preferred. Specific examples of the sulfonium cation include those having the formula (Z-1), those described in in JP-A 2024-003744,

paragraphs to [0125], those described in JP-A 2023-169812, paragraphs to

, and those having the formula (Z-3), but are not limited thereto. Specific examples

of the iodonium cation include those having the formula (Z-2), and those described in JP-A 2024-000259, paragraph [0181], but are not limited thereto. Specific examples of the ammonium cation are as exemplified later for the ammonium cation having the formula (am-1), but are not limited thereto.

Specific examples of structures of the repeat units (c1) to (c5) include arbitrary combinations of anions with cations, both as exemplified above.

Among the repeat units (c1) to (c5), the repeat units (c2) to (c5) are preferred from the aspect of controlling acid diffusion, the repeat units (c2), (c4), and (c5) are more preferred from the aspect of the acid strength of the generated acid, and the repeat unit (c2) is still more preferred from the aspect of solvent solubility.

When the chemically amplified resist composition of the invention is of positive tone, the base polymer may contain a repeat unit having a structure in which a hydroxy group is protected with an acid labile group (hereinafter also referred to as repeat unit (d)). The repeat unit (d) is not particularly limited as long as it has one or more structures having a hydroxy group protected with a protective group such that the protective group is decomposed to generate a hydroxy group under the action of an acid, and a repeat unit having the formula (d1) is preferred.

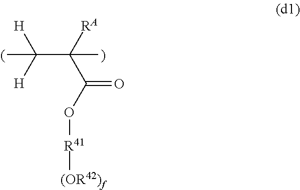

In the formula (d1), R^{A} is as defined above. R⁴¹ is a C₁-C₃₀ (f+1)-valent hydrocarbon group which may contain a heteroatom. R⁴² is an acid labile group. f is 1, 2, 3, or 4.

In the formula (d1), the acid labile group of R⁴² may be any group that is deprotected by the action of an acid to generate a hydroxy group. The structure of R⁴² is not particularly limited, but is preferably an acetal structure, a ketal structure, an alkoxycarbonyl group, an alkoxymethyl group having the formula (d2), or the like, and particularly preferably an alkoxymethyl group having the formula (d2).

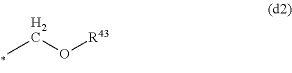

In the formula, * designates a point of attachment. R⁴³ is a C₁-C₁₅ hydrocarbyl group.

Specific examples of the acid labile group of R⁴², the alkoxymethyl group having the formula (d2), and the repeat unit (d) are as exemplified for the repeat unit (d) described in JP-A 2020-111564.

The base polymer may further contain a repeat unit (e) derived from indene, benzofuran, benzothiophene, acenaphthylene, chromone, coumarin, norbornadiene, or a derivative thereof. Specific examples of the monomer that gives the repeat unit (e) are shown below, but not limited thereto.

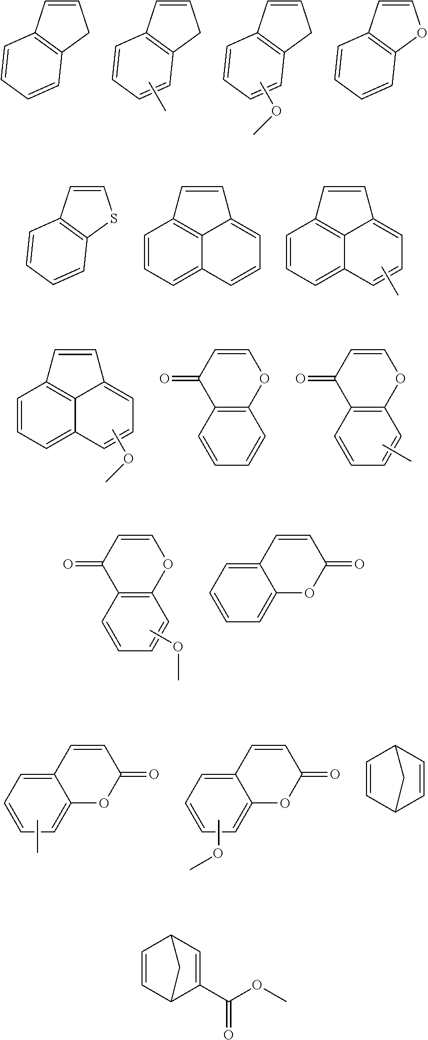

The base polymer may further contain a repeat unit (f) derived from indane, vinylpyridine, or vinylcarbazole.

The base polymer for a positive resist composition essentially contains any of the repeat units (a1) to (a3) containing an acid labile group. In this case, the content ratios of the repeat units (a1), (a2), (a3), (b1), (b2), (c1) to (c5), (d), (e), and (f) in the base polymer are preferably 0<a1≤0.8,0≤a2≤0.8,0≤a3≤0.6,0≤b1≤0.6,0≤b2≤0.6,0≤c1≤0.4, 0≤c2≤0.4, 0≤c3≤0.4, 0≤c4≤0.4, 0≤c5≤0.4, 0≤d≤0.5, 0≤e≤0.3, and 0≤f≤0.3, and more preferably 0<a1≤0.7,0≤a2≤0.7,0≤a3≤0.5,0≤b1≤0.5,0≤b2≤0.5, 0≤c1≤0.3, 0≤c2≤0.3, 0≤c3≤0.3, 0≤c4≤0.3, 0≤c5≤0.3, 0≤d≤0.3, 0≤e≤0.3, and 0≤f≤0.3. These subscripts meet al+a2+a3+b1+b2+c1+c2+c3+c4+c5+d+e+f≤1.0. On the other hand, the base polymer for a negative resist composition does not contain the repeat units (a1) to (a3) and (d) but contains the repeat unit (a) 4. In this case, the content ratios of the repeat units (a4), (b1), (b2), (c1) to (c5), (e), and (f) in the base polymer are preferably 0<a4≤0.7, 0≤b1≤0.5, 0≤b2≤0.7, 0≤c1≤0.4, 0≤c2≤0.4, 0≤c3≤0.4, 0≤c4≤0.4, 0≤c5≤0.4, 0≤e≤0.3, and 0≤f≤0.3, and more preferably 0<a4≤0.6, 0≤b1≤0.4, 0≤b2≤0.6, 0≤c1≤0.3, 0≤c2≤0.3, 0≤c3≤0.3, 0≤c4≤0.3, 0≤c5≤0.3, 0 Se≤0.3, and 0≤f≤0.3. These subscripts meet a4+b1+b2+c1+c2+c3+c4+c5+e+f≤1.0.

The polymer preferably has a weight average molecular weight (Mw) of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. In the invention, Mw is a value measured in terms of polystyrene by gel permeation chromatography (GPC) using THF or N,N-dimethylformamide (DMF) as a solvent.

As for the molecular weight distribution (Mw/Mn) of the polymer, since the influence of Mw/Mn tends to become stronger as the pattern rule becomes finer, the polymer preferably has a narrow dispersity (Mw/Mn) of 1.0 to 2.0 in order to provide a resist composition suitable for fine pattern dimensions. A Mw/Mn in the range indicates smaller amounts of lower and higher molecular weight fractions and eliminates the risk of leaving foreign matter on the pattern or degrading the pattern profile after exposure.

The polymer may be synthesized by, for example, adding a radical polymerization initiator and heating and polymerizing a monomer that gives the above-mentioned repeat unit in an organic solvent.

Specific examples of the organic solvent used for the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers to be polymerized. The reaction temperature is preferably 50 to 150° C., and more preferably 60 to 100° C. The reaction time is preferably 2 to 24 hours, and more preferably 2 to 12 hours from the aspect of production efficiency.

The polymerization initiator may be added to the monomer solution, which is fed to the reaction vessel, or an initiator solution may be prepared separately from the monomer solution and fed to the reaction vessel independently. Since a polymerization reaction may proceed to form an ultrahigh molecular weight compound due to a radical generated from the initiator in the standby time, it is preferred from the aspect of quality control that the monomer solution and the initiator solution be independently prepared and added dropwise. The acid labile group that has been incorporated in the monomer may be kept as such, or the polymerization may be followed by protection or partial protection. A known chain transfer agent such as dodecylmercaptan or 2-mercaptoethanol may be used for the purpose of adjusting the molecular weight. In that case, the amount of such a chain transfer agent added is preferably 0.01 to 20 mol % based on the total of monomer to be polymerized.

In the case of a monomer containing a hydroxy group, the hydroxy group may be substituted with an acetal group susceptible to deprotection with an acid such as an ethoxyethoxy group during polymerization, and then deprotected with a weak acid and water after polymerization, or may be substituted with an acetyl group, a formyl group, a pivaloyl group, or the like, and then alkaline hydrolysis may be performed after polymerization.

When hydroxystyrene or hydroxyvinylnaphthalene is copolymerized, hydroxystyrene or hydroxyvinylnaphthalene and another monomer may be heated and polymerized by adding a radical polymerization initiator in an organic solvent, but acetoxystyrene or acetoxyvinylnaphthalene may be used, and the acetoxy group may be deprotected by alkaline hydrolysis after polymerization to form polyhydroxystyrene or hydroxypolyvinylnaphthalene.

As the base during alkaline hydrolysis, ammonia water, triethylamine, or the like can be used. The reaction temperature is preferably-20 to 100° C., and more preferably 0 to 60° C. The reaction time is preferably 0.2 to 100 hours, and more preferably 0.5 to 20 hours. The amount of each monomer in the monomer solution may be appropriately set, for example, so as to provide the preferred content ratios of the repeat units described above.

As for the polymer obtained by the production method, a reaction solution obtained by a polymerization reaction may be used as a final product, or a powder obtained through a purification step such as a reprecipitation method in which a polymerization liquid is added to a poor solvent to obtain a powder may be handled as a final product. From the aspect of operation efficiency and consistent quality, it is preferred to handle a polymer solution obtained by dissolving the powder obtained by the purification step in a solvent as a final product.

Specific examples of the solvent used at that time include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); high-boiling alcoholic solvents such as diethylene glycol, propylene glycol, glycerol, 1,4-butanediol, and 1,3-butanediol; and mixed solvents thereof described in JP-A 2008-111103, paragraphs to [0145].

The concentration of the polymer in the polymer solution is preferably 0.01 to 30 mass %, and more preferably 0.1 to 20 mass %.

The reaction solution and the polymer solution are preferably filtered through a filter. Filtration is effective for consistent quality because foreign matter and gel which can cause defects can be removed.

Examples of the material of the filter used for the filtration include fluorocarbon, cellulose, nylon, polyester, and hydrocarbon base materials, and in the filtering step of the resist composition, a filter formed of a fluorocarbon base material called Teflon®, a hydrocarbon base material such as polyethylene or polypropylene, or nylon is preferred. The pore size of the filter can be appropriately selected according to the desired cleanliness, but is preferably 100 nm or less, and more preferably 20 nm or less. One type of filter may be used alone or a plurality of filters may be used in combination. Although the filtering method may be single pass of the solution, it is more preferred to perform filtration a plurality of times by circulating the solution. In the polymer preparation process, the filtering step may be performed any times, in any order, but it is preferred to filter the reaction solution after the polymerization reaction, the polymer solution, or both.

As the base polymer (B), one type may be used alone or two or more types which are different in compositional ratio, Mw, and/or Mw/Mn may be combined and used. In addition, the base polymer (B) may contain a hydrogenated product of a ring-opening metathesis polymer, and as the hydrogenated product, those described in JP-A 2003-66612 can be used.

### (C) Organic Solvent

The chemically amplified resist composition of the invention may contain an organic solvent as a component (C). The organic solvent (C) is not particularly limited as long as it can dissolve the above and below-mentioned components. Specific examples of such an organic solvent include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and mixed solvents thereof.

Among these organic solvents, 1-ethoxy-2-propanol, PGMEA, cyclohexanone, GBL, DAA, and mixed solvents thereof are preferred because the solubility of the base polymer of the component (B) therein is particularly high.

In the chemically amplified resist composition of the invention, the content of the organic solvent (C) is preferably 200 to 5,000 parts by weight, and more preferably 400 to 3,500 parts by weight per 80 parts by weight of the base polymer (B). As the organic solvent (C), one type may be used alone or two or more types may be mixed and used.

### (D) Photoacid Generator

The chemically amplified resist composition of the invention may contain a photoacid generator as a component (D). The photoacid generator is not particularly limited as long as it is a compound that generates an acid by irradiation with high-energy radiation. Preferred examples of the photoacid generator include photoacid generators having the formula (2) or (3).

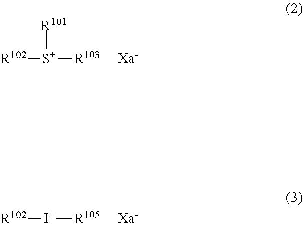

In the formula (2), R¹⁰¹ to R¹⁰⁵ are each independently a C₁-C₂₀ hydrocarbyl group which may contain a heteroatom, Any two of R¹⁰¹, R¹⁰², and R¹⁰³ may bond together to form a ring with the sulfur atom to which they are attached.

Specific examples of the cation of the sulfonium salt having the formula (2) and the cation of the iodonium salt having the formula (3) are as exemplified above as specific examples of the sulfonium cation and the iodonium cation of Z⁺ in the description of the formula (1), but are not limited thereto.

In the formulae (2) and (3), Xa is an anion of a strong acid. Examples of the anion of a strong acid include those having the formulae (c1-1) to (c1-5).

As the photoacid generator of the component (D), a photoacid generator having the formula (4) is also preferred.

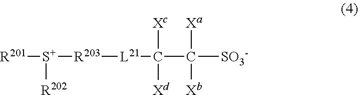

In the formula (4), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may bond together to form a ring with the sulfur atom to which they are attached.

The C₁-C₃₀ hydrocarbyl group of R²⁰¹ and R²⁰² may be saturated or unsaturated and straight, branched, or cyclic. Specific examples thereof include C₁-C₃₀ alkyl groups such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, and a n-decyl group; C₃-C₃₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.02.6] decyl group, and an adamantyl group; C₆-C₃₀ aryl groups such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, and an anthracenyl group; and a group obtained by combining these groups. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

The C₁-C₃₀ hydrocarbylene group of R²⁰³ may be saturated or unsaturated and straight, branched, or cyclic. Specific examples thereof include C₁-C₃₀ alkanediyl groups such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; C₃-C₃₀ cyclic saturated hydrocarbylene groups such as a cyclopentanediyl group, a cyclohexanediyl group, a norbornanediyl group, and an adamantanediyl group; and arylene groups such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, and a tert-butylnaphthylene group. Some or all hydrogen atoms in the hydrocarbylene group may be substituted with a group containing a heteroatom such as an oxygen atom, a sulfur atom, a nitrogen atom, or a halogen atom, and some —CH₂-in the hydrocarbylene group may be substituted with a group containing a heteroatom such as an oxygen atom, a sulfur atom, or a nitrogen atom, so that the hydrocarbylene group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. The heteroatom is preferably an oxygen atom.

In the formula (4), L²¹ is a single bond, an ether bond, or a C₁-C₂₀ hydrocarbylene group which may contain a heteroatom. The hydrocarbylene group may be saturated or unsaturated and straight, branched, or cyclic. Specific examples thereof are as exemplified above for the hydrocarbylene group of R²⁰³.

In the formula (4), Xa, Xb, X°, and Xd are each independently a hydrogen atom, a fluorine atom, or a trifluoromethyl group. However, at least one of Xª, Xb, Xe, and Xd is a fluorine atom or a trifluoromethyl group.

As the photoacid generator having the formula (4), a photoacid generator having the formula (4′) is preferred.

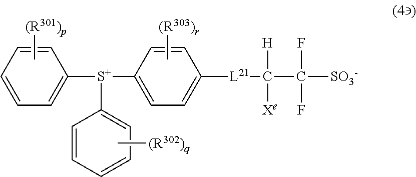

In the formula (4′), L²¹ is as defined above. Xe is a hydrogen atom or a trifluoromethyl group, preferably a trifluoromethyl group. R³⁰¹, R³⁰², and R³⁰³ are each independently a hydrogen atom or a C₁-C₂₀ hydrocarbyl group which may contain a heteroatom. The hydrocarbyl group may be saturated or unsaturated and straight, branched, or cyclic. Specific examples thereof are as exemplified above for the hydrocarbyl group of R^{fa1} in the formula (c1-1-1). p and q are each independently 0, 1, 2, 3, 4, or 5, and
r is 0, 1, 2, 3, or 4.

Examples of the photoacid generator having the formula (4) are as exemplified for the photoacid generator having the formula (2) in JP-A 2017-26980.

Among the photoacid generators, those containing an anion having the formula (c1-1-1) or (c1-4) are particularly preferred because of reduced acid diffusion and high solubility in the solvent. In addition, those having the formula (4′) are particularly preferred because of extremely reduced acid diffusion.

When the chemically amplified resist composition of the invention contains the photoacid generator (D), the content thereof is preferably 0.1 to 40 parts by weight, and more preferably 0.5 to 20 parts by weight per 80 parts by weight of the base polymer (B). The amount of the photoacid generator as the component (D) in the above range is preferred because a satisfactory resolution is available without any possibility that a problem of foreign matter occurs after development or during peeling of the resist film. As the photoacid generator (D), one type may be used alone or two or more types may be combined and used.

### (E) Other Quencher

The chemically amplified resist composition of the invention may contain a quencher other than the component (A) as a component (E).

Examples of the quencher (E) include an onium salt having the formula (5) or (6).

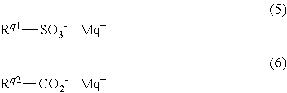

In the formula (5), R⁹¹ is a hydrogen atom or a C₁-C₄₀ hydrocarbyl group which may contain a heteroatom, exclusive of a group in which a hydrogen atom attached to a carbon atom at α-position of a sulfo group is substituted with a fluorine atom or a fluoroalkyl group. In the formula (6), R⁹² is a hydrogen atom or a C₁-C₄₀ hydrocarbyl group which may contain a heteroatom.

Specific examples of the C₁-C₄₀ hydrocarbyl group of R⁴¹ include C₁-C₄₀ alkyl groups such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; C₃-C₄₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.02.6] decyl group, and an adamantyl group; and C₆-C₄₀ aryl groups such as a phenyl group, a naphthyl group, and an anthracenyl group. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be replaced by a group containing a heteroatom such as an oxygen atom, a sulfur atom, or a nitrogen atom, so that the hydrocarbyl group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Specific examples of the hydrocarbyl group of R⁹² include, in addition to the substituents exemplified above as specific examples of R⁹¹, fluorinated saturated hydrocarbyl groups such as a trifluoromethyl group and a trifluoroethyl group, and fluorinated aryl groups such as a pentafluorophenyl group and a 4-trifluoromethylphenyl group.

Specific examples of the anion of the onium salt having the formula (5) are shown below, but not limited thereto.

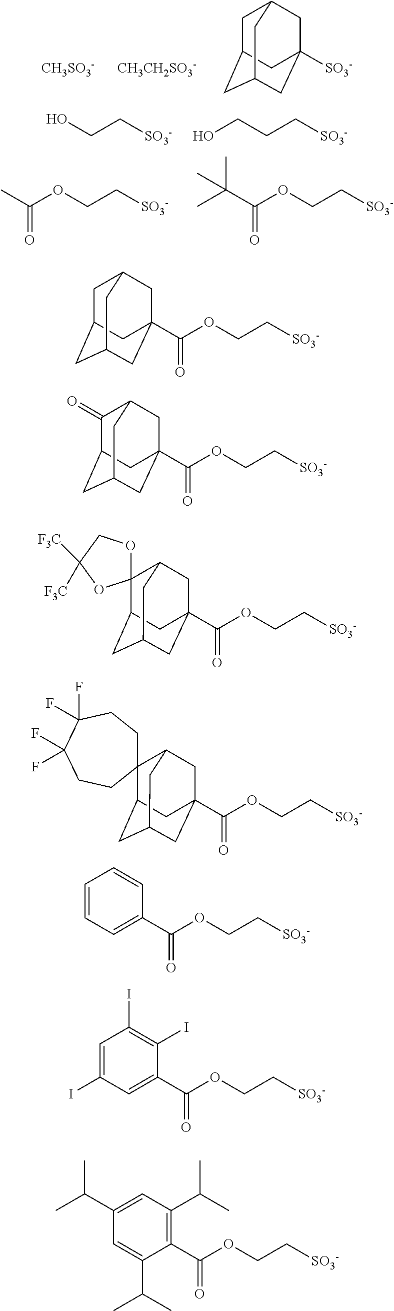

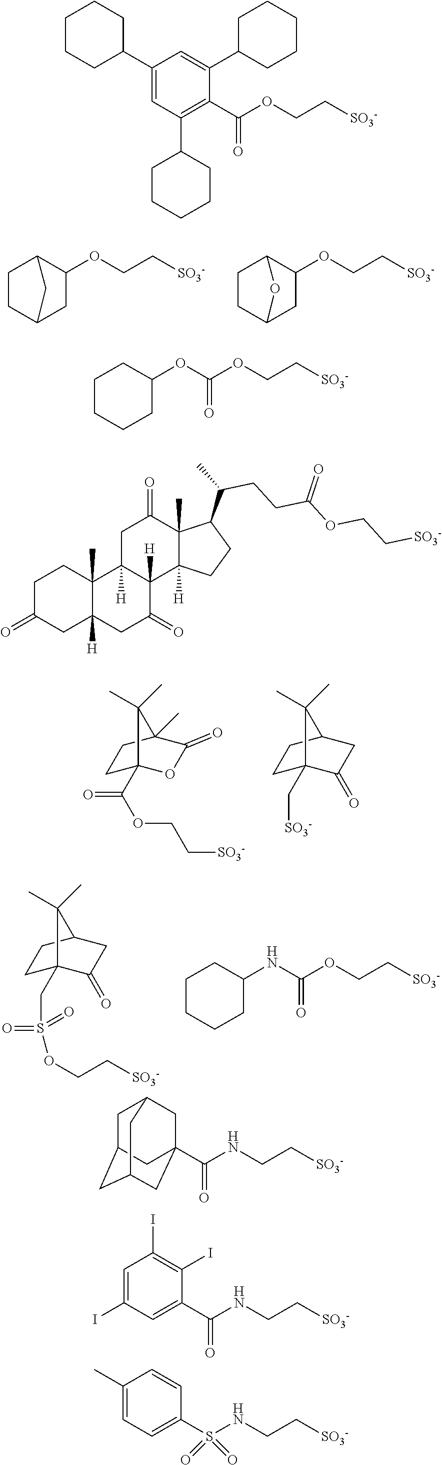

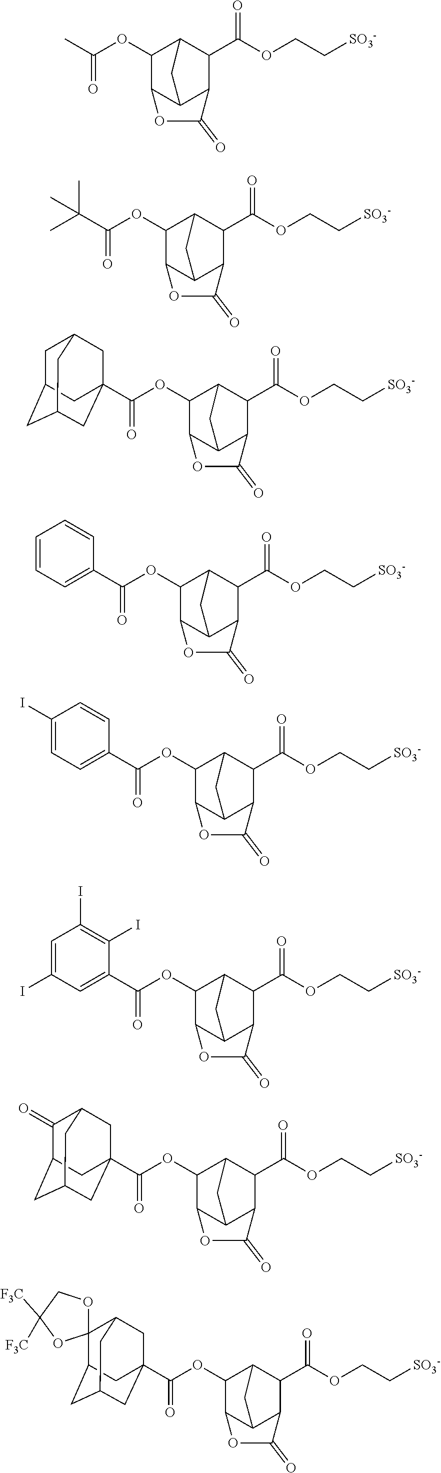

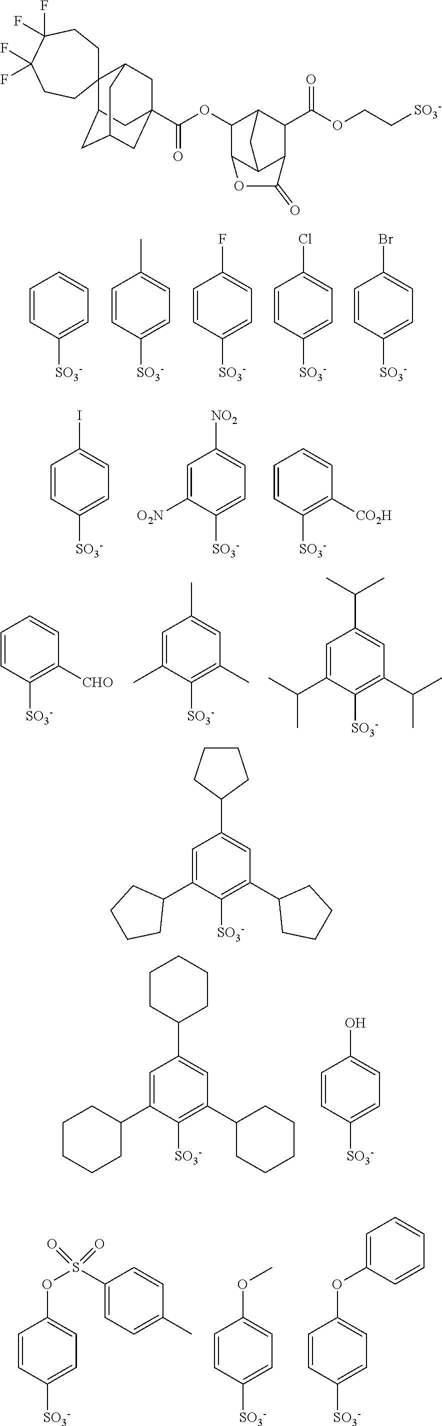

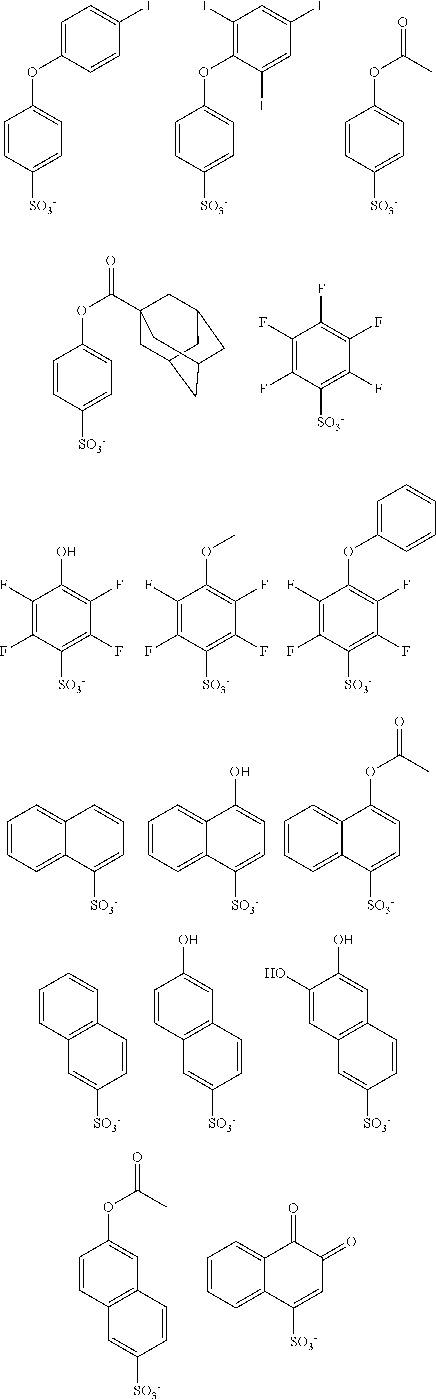

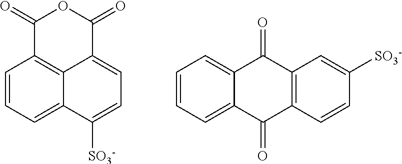

1.

Specific examples of the anion of the onium salt having the formula (6) are shown below, but not limited thereto.

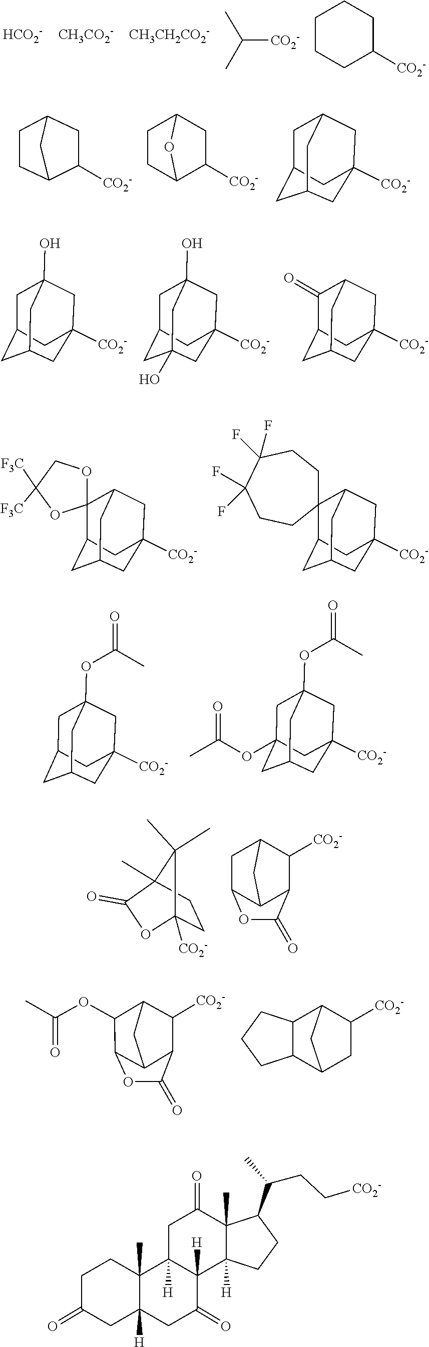

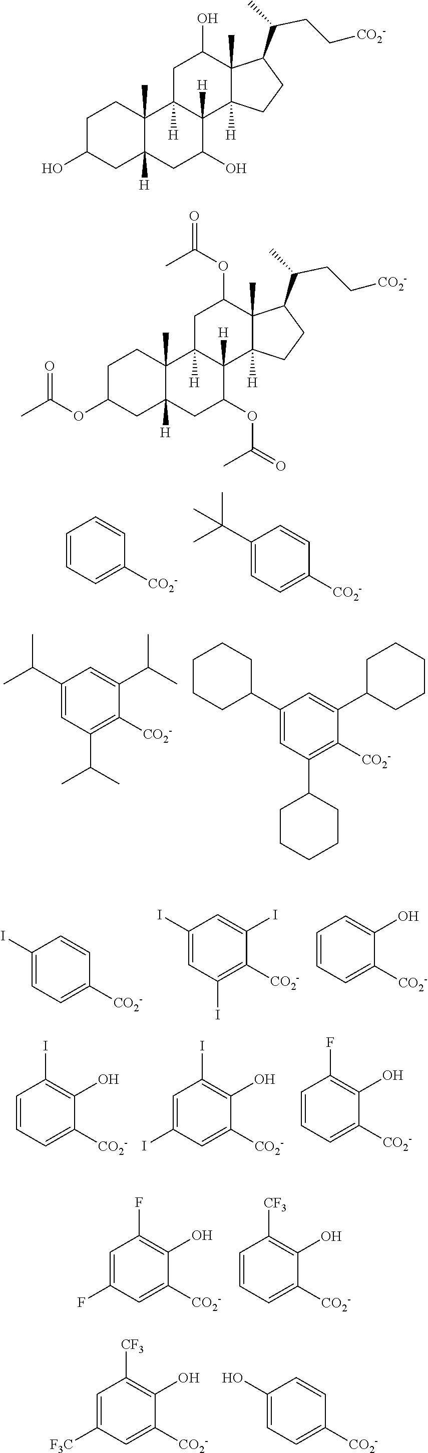

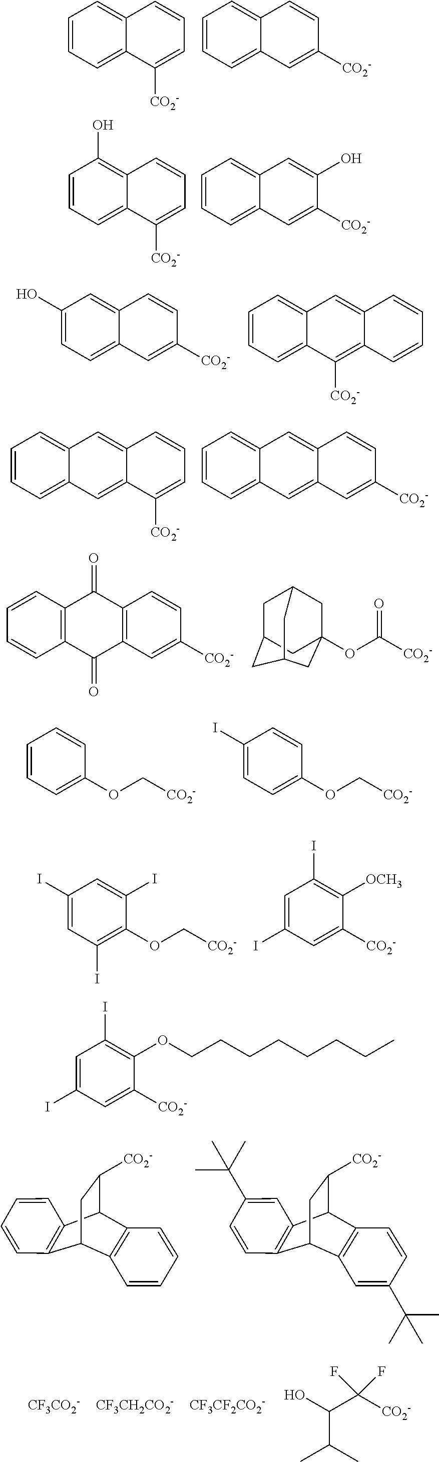

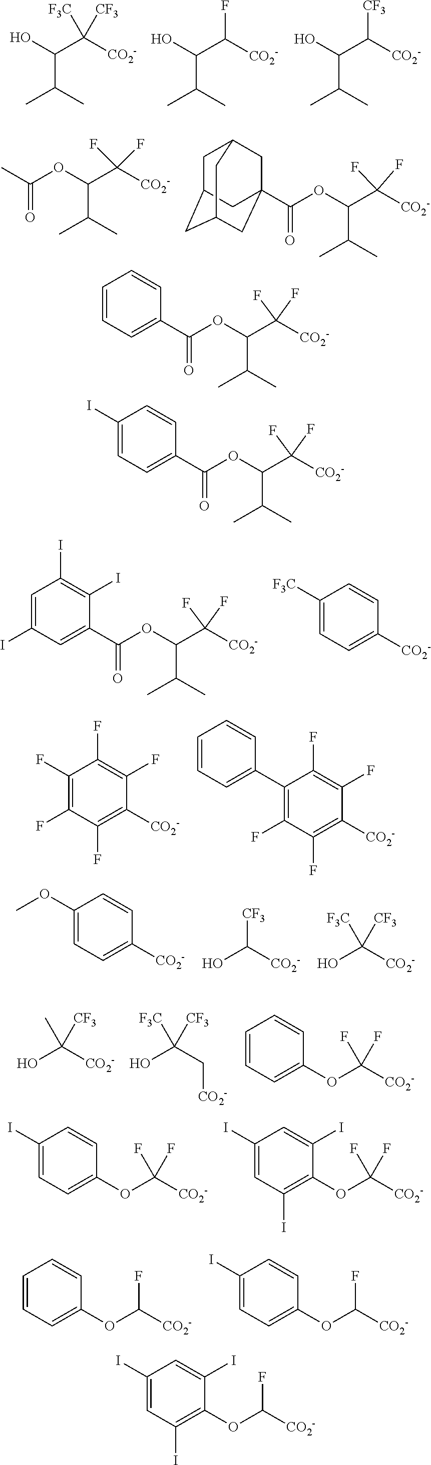

In the formula (5) or (6), Mq⁺ is an onium cation. Examples of the onium cation include a sulfonium cation, an iodonium cation, and an ammonium cation. Specific examples of the sulfonium cation, the iodonium cation, and the ammonium cation are as exemplified above as specific examples of the sulfonium cation, the iodonium cation, and the ammonium cation of Z⁺ in the description of the formula (1), but are not limited thereto.

Specific examples of the onium salt having the formula (5) or (6) include arbitrary combinations of anions with cations, both as exemplified above. These onium salts are easily prepared by an ion exchange reaction using a known organic chemical method. With respect to the ion exchange reaction, reference may be made to, for example, JP-A 2007-145797.

The onium salt having the formula (5) or (6) acts as a quencher in the chemically amplified resist composition of the invention. This is because each counter anion of the onium salt is a conjugate base of a weak acid. The weak acid as used herein means an acid having an acidity insufficient to deprotect an acid labile group of an acid labile group-containing unit in the base polymer. The onium salt having the formula (5) or (6) functions as a quencher when used in combination with an onium salt type photoacid generator having a conjugated base of a strong acid such as a sulfonic acid fluorinated at α-position as the counter anion. That is, when an onium salt that generates a strong acid such as a sulfonic acid fluorinated at α-position and an onium salt that generates a weak acid such as a sulfonic acid or a carboxylic acid that is not fluorinated are mixed and used, if the strong acid generated from the photoacid generator by irradiation with high-energy radiation collides with an unreacted onium salt having a weak acid anion, the weak acid is released by salt exchange to form an onium salt having a strong acid anion. In this process, the strong acid is exchanged with the weak acid having a lower catalysis, so that the acid is apparently deactivated and acid diffusion can be controlled.

As the other quencher (E), an onium salt having sulfonium cation and phenoxide anion sites in a common molecule described in JP 6848776, an onium salt having sulfonium cation and carboxylate anion sites in a common molecule described in JP 6583136 and JP-A 2020-200311, and an onium salt having iodonium cation and carboxylate anion sites in a common molecule described in JP 6274755 can also be used.

Here, when the photoacid generator that generates a strong acid is an onium salt, as described above, the strong acid generated by irradiation with high-energy radiation can be exchanged with a weak acid, but it is considered difficult for a weak acid generated by irradiation with high-energy radiation to collide with an unreacted onium salt that generates a strong acid to induce salt exchange. This is due to the phenomenon that an onium cation easily forms an ion pair with a stronger acid anion.

When the chemically amplified resist composition of the invention contains the onium salt having the formula (5) or (6) as the other quencher (E), the content thereof is preferably 0.1 to 20 parts by weight, and more preferably 0.1 to 10 parts by weight per 80 parts by weight of the base polymer (B). The content of the onium salt type quencher of the component (E) in the above range is preferred because a satisfactory resolution is available without a substantial lowering of the sensitivity. As the onium salt having the formula (5) or (6), one type may be used alone or two or more types may be combined and used.

The chemically amplified resist composition of the invention may contain a nitrogen-containing compound as the other quencher (E). Specific examples of the nitrogen-containing compound as the component (E) include primary, secondary, or

tertiary amine compounds described in JP-A 2008-111103, paragraphs to [0164], particularly amine compounds having a hydroxy group, an ether bond, an ester bond, a lactone ring, a cyano group, or a sulfonate ester bond. Additional examples include a compound obtained by protecting a primary or secondary amine with a carbamate group as described in JP 3790649.

A sulfonic acid sulfonium salt having a nitrogen-containing substituent may also be used as the nitrogen-containing compound. Such a compound functions as a quencher in an unexposed portion, and as a so-called photo-degradable base that loses the quencher ability due to neutralization with the acid generated by itself in an exposed portion. Use of a photo-degradable base can further enhance the contrast between the exposed portion and the unexposed portion. With respect to the photo-degradable base, reference may be made to, for example, JP-A 2009-109595 and JP-A 2012-46501.

When the chemically amplified resist composition of the invention contains a nitrogen-containing compound as the other quencher (E), the content thereof is preferably 0.001 to 12 parts by weight, and more preferably 0.01 to 8 parts by weight per 80 parts by weight of the base polymer (B). As the nitrogen-containing compound, one type may be used alone or two or more types may be combined and used.

### (F) Surfactant

The chemically amplified resist composition of the invention may contain a surfactant as a component (F). The surfactant (F) is preferably a surfactant insoluble or sparingly soluble in water and soluble in an alkaline developer, or a surfactant insoluble or sparingly soluble in water and an alkaline developer. With respect to such a surfactant, reference may be made to those compounds described in JP-A 2010-215608 and JP-A 2011-16746.

As the surfactant insoluble or sparingly soluble in water and an alkaline developer, among the surfactants described in the above-mentioned patent documents, FC-4430 (manufactured by 3M), Surflon® S—381 (manufactured by AGC Seimi Chemical Co., Ltd.), Olfine® E1004 (manufactured by Nissin Chemical Industry Co., Ltd.), KH-20, KH-30 (manufactured by AGC Seimi Chemical Co., Ltd.), an oxetane ring-opening polymer having the formula (surf-1), and the like are preferred.

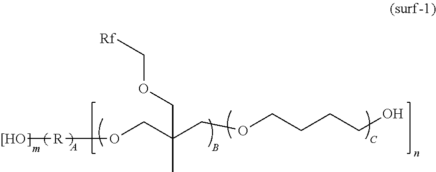

Here, R, Rf, A, B, C, m, n are applied only to the formula (surf-1) regardless of the above description. R is a di- to tetra-valent C₂-C₅ aliphatic group. Examples of the divalent aliphatic group include an ethylene group, a 1,4-butylene group, a 1,2-propylene group, a 2,2-dimethyl-1,3-propylene group, and a 1,5-pentylene group, and examples of the trivalent or tetravalent aliphatic group include the following groups.

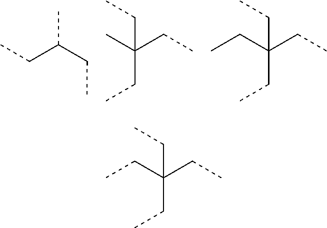

In the formulae, broken lines are points of attachment and are partial structures derived from glycerol, trimethylolethane, trimethylolpropane, and pentaerythritol, respectively.

Among them, a 1,4-butylene group, a 2,2-dimethyl-1,3-propylene group, and the like are preferred.

Rf is a trifluoromethyl group or a pentafluoroethyl group, and preferably a trifluoromethyl group. m is an integer of 0 to 3, n is an integer of 1 to 4, and the sum of n and m is the valence of R and an integer of 2 to 4. A is 1. B is an integer of 2 to 25, and preferably an integer of 4 to 20. C is an integer of 0 to 10, and preferably 0 or 1. The formula (surf-1) does not prescribe the arrangement of respective constituent units while the units may be arranged either blockwise or randomly. The production of a partially fluorinated oxetane ring-opening polymer-based surfactant is described in detail in U.S. Pat. No. 5,650,483 and the like.

When a resist protective film is not used in ArF immersion lithography, a surfactant insoluble or sparingly soluble in water and soluble in an alkaline developer has a function of reducing water penetration or leaching by orientation on the surface of the resist film. Therefore, such a surfactant is useful for inhibiting elution of a water-soluble component from the resist film to reduce damage to an exposure apparatus, and is also useful because such a surfactant becomes soluble during development with an alkaline aqueous solution after the exposure or after post exposure bake (PEB), and hardly forms foreign matter causing a defect. The preferred surfactant is a polymeric surfactant which is insoluble or sparingly soluble in water and soluble in an alkaline developer, also referred to as “hydrophobic resin”, and especially is water repellent and enhances water sliding.

Specific examples of such a polymeric surfactant include those containing at least one repeat unit selected from repeat units having the formulae (7A) to (7E).

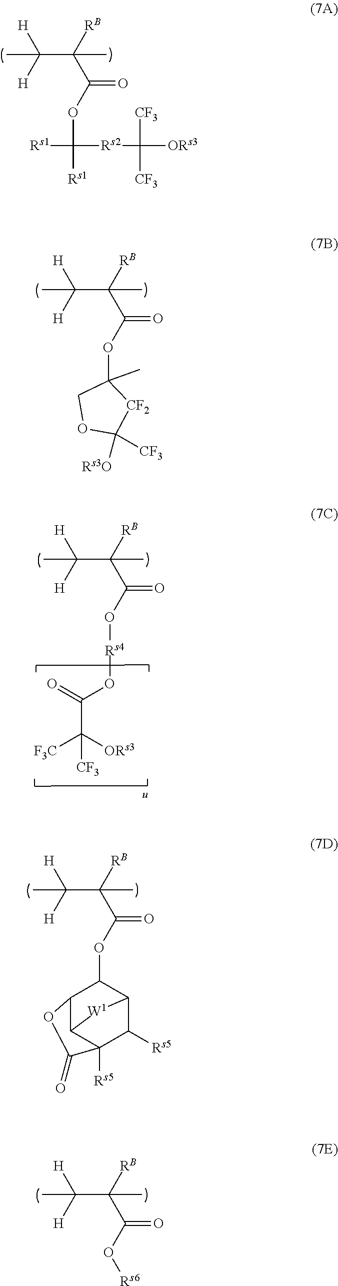

In the formulae (7A) to (7E), RB is a hydrogen atom, a fluorine atom, a methyl group, or a trifluoromethyl group. W1 is —CH₂—, —CH₂CH₂—, —O—, or two separate-H. R^{s1}'s are each independently a hydrogen atom or a C₁-C₁₀ hydrocarbyl group. R^{s2} is a single bond or a C₁-C₅ straight or branched hydrocarbylene group. R^{s3}'s are each independently a hydrogen atom, a C₁-C₁₅ hydrocarbyl group, a fluorinated hydrocarbyl group, or an acid labile group. When R^{s3} is a hydrocarbyl group or a fluorinated hydrocarbyl group, an ether bond or a carbonyl group may intervene in a carbon-carbon bond. R^{s4} is a C₁-C₂₀ (u+1)-valent hydrocarbon group or fluorinated hydrocarbon group. u is 1, 2, or 3. R^{s5}'s are each independently a hydrogen atom or a group of —C(═O)—O—R^{sa}. R^{sa} is a C₁-C₂₀ fluorinated hydrocarbyl group. R^{s6} is a C₁-C₅ is hydrocarbyl group or a fluorinated hydrocarbyl group, and an ether bond or a carbonyl group may intervene in a carbon-carbon bond.

The C₁-C₁₀ hydrocarbyl group of R^{s1} is preferably a saturated hydrocarbyl group, and may be straight, branched, or cyclic. Specific examples thereof include C₁-C₁₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, and a n-decyl group; and C₃-C₁₀ cyclic saturated hydrocarbyl groups such as a cyclopropyl group, a cyclobutyl group, a cyclopentyl group, a cyclohexyl group, an adamantyl group, and a norbornyl group. Among them, C₁-C₆ groups are preferred.

The hydrocarbylene group of R^{s2} is preferably a saturated hydrocarbylene group, and may be straight, branched, or cyclic. Specific examples thereof include a methylene group, an ethylene group, a propylene group, a butylene group, and a pentylene group.

The hydrocarbyl group of R^{s3} or R^{s6} may be saturated or unsaturated and straight, branched, or cyclic. Specific examples thereof include saturated hydrocarbyl groups and aliphatic unsaturated hydrocarbyl groups such as an alkenyl group and an alkynyl group, and saturated hydrocarbyl groups are preferred. Examples of the saturated hydrocarbyl group include an undecyl group, a dodecyl group, a tridecyl group, a tetradecyl group, and a pentadecyl group in addition to those exemplified above for the hydrocarbyl group of R^{s1}. Examples of the fluorinated hydrocarbyl group of R^{s3} or R^{s6} include groups in which some or all hydrogen atoms attached to carbon atoms of the above-mentioned hydrocarbyl group are substituted with fluorine atoms. As described above, an ether bond or a carbonyl group may intervene in a carbon-carbon bond.

Specific examples of the acid labile group of R^{s3} include groups having the formulae (AL-3) to (AL-5), a trialkylsilyl group in which each alkyl group is a C₁-C₆ alkyl group, and C₄-C₂₀ oxoalkyl groups.

The (u+1)-valent hydrocarbon group or fluorinated hydrocarbon group of R^{s4} may be straight, branched, or cyclic, and specific examples thereof include groups obtained by further eliminating “u” number of hydrogen atoms from the above-mentioned hydrocarbyl groups, fluorinated hydrocarbyl groups, and the like.

The fluorinated hydrocarbyl group of R^{sa} is preferably a saturated one, and may be straight, branched, or cyclic. Specific examples thereof include hydrocarbyl groups in which some or all hydrogen atoms in the hydrocarbyl group are substituted with fluorine atoms, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeat units having the formulae (7A) to (7E) are shown below, but not limited thereto. In the following formulae. RB is as defined above.

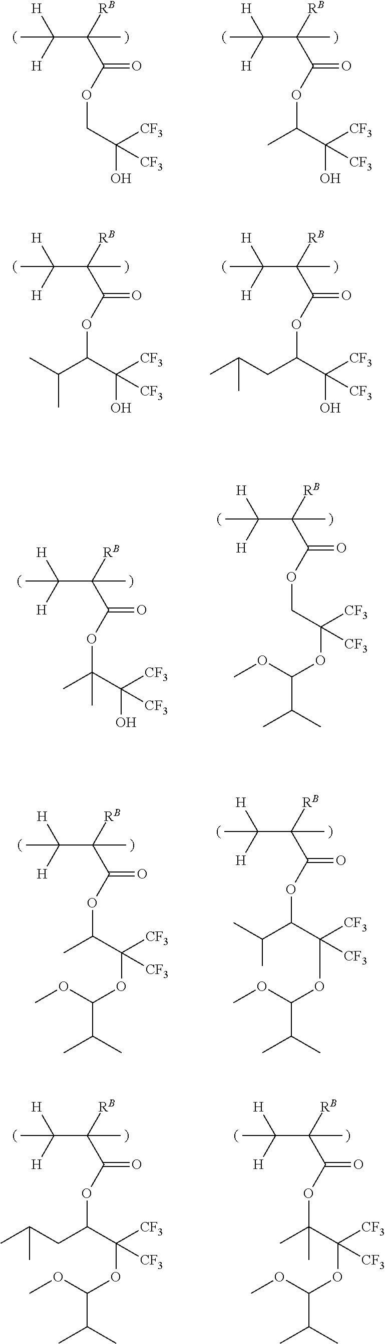

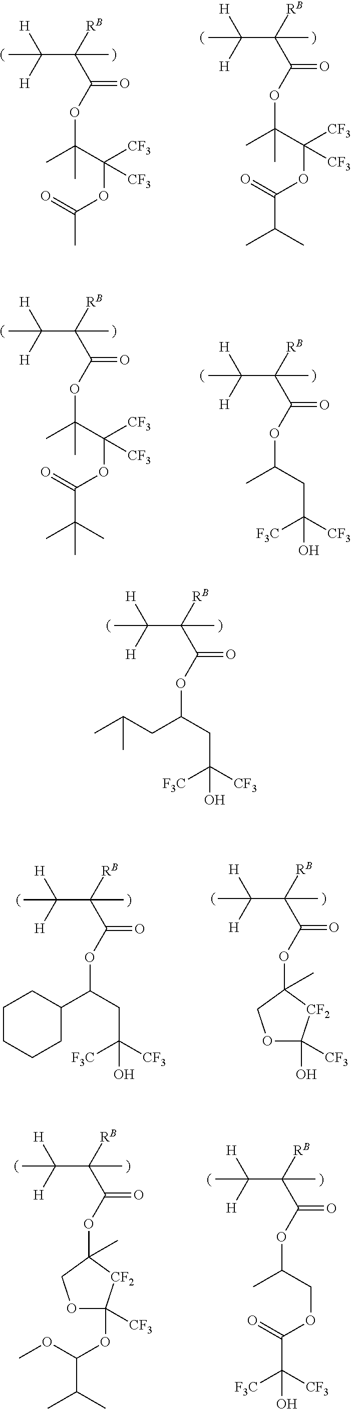

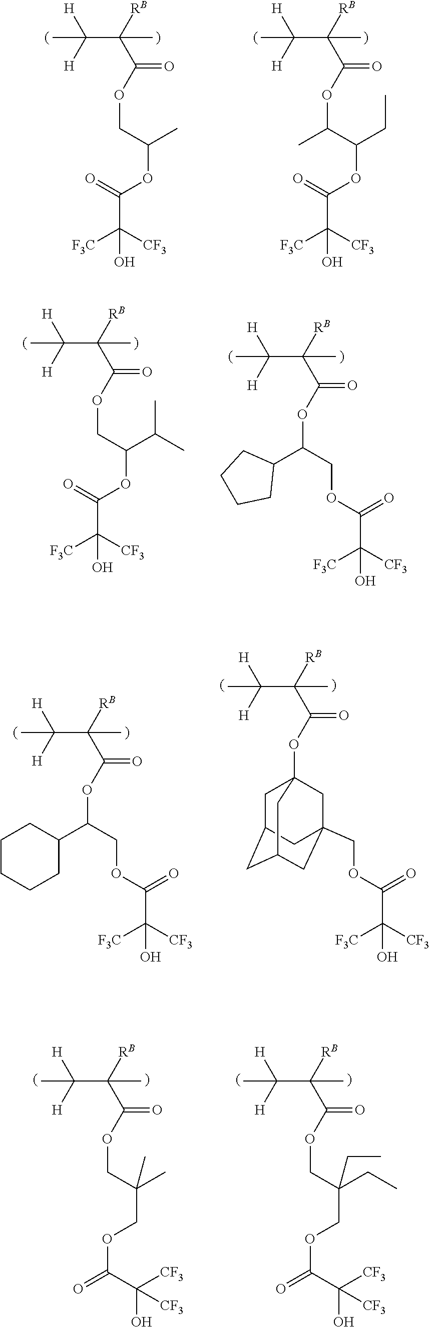

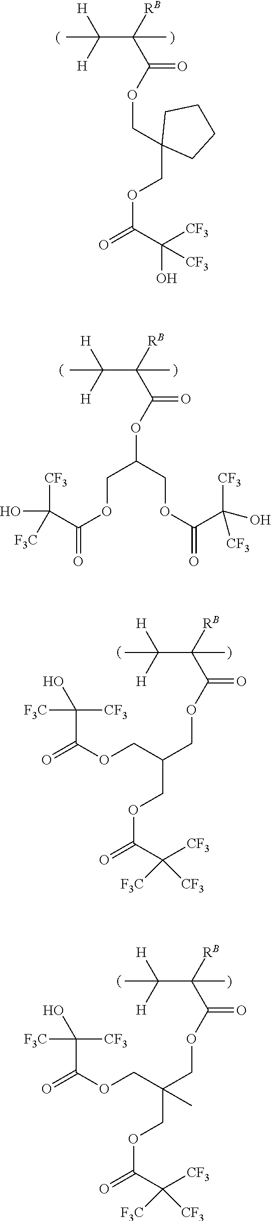

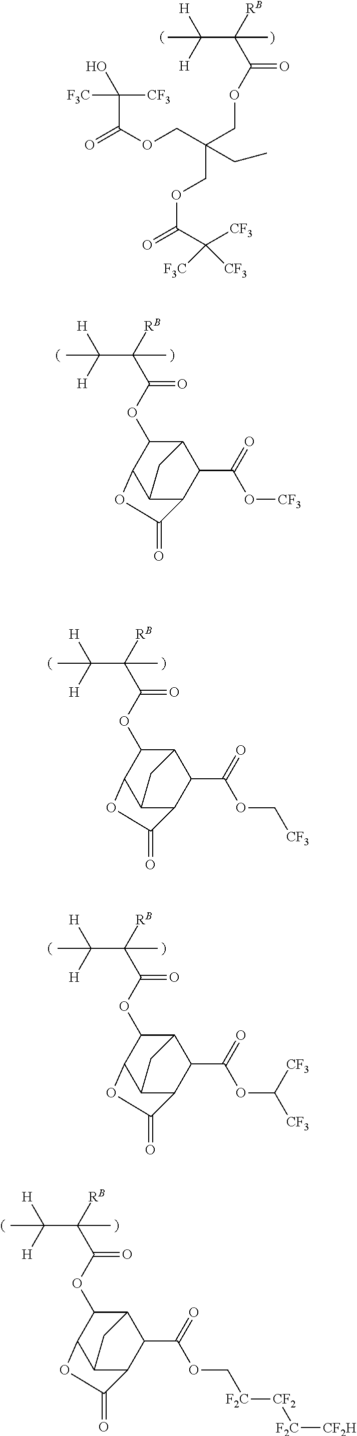

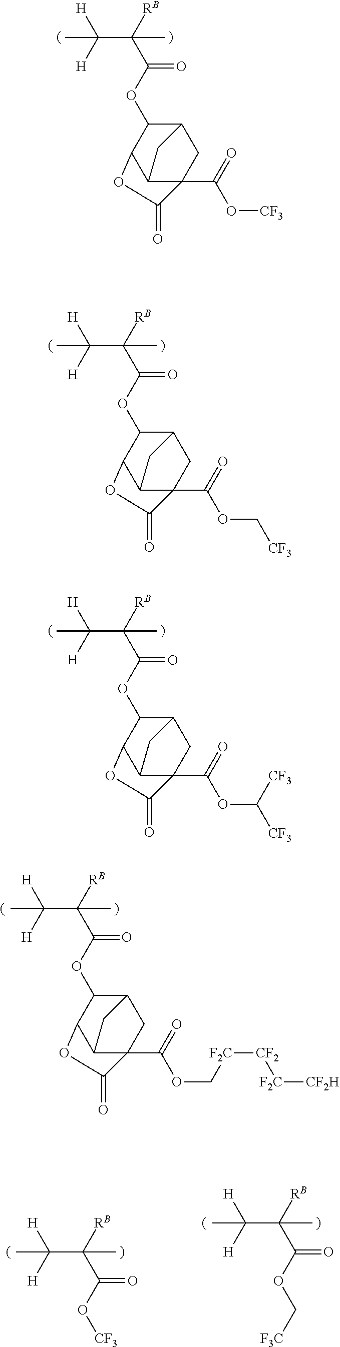

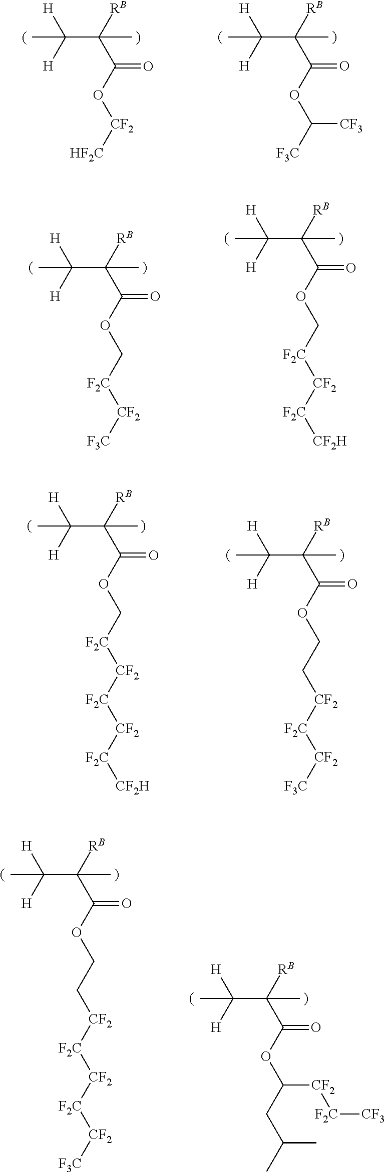

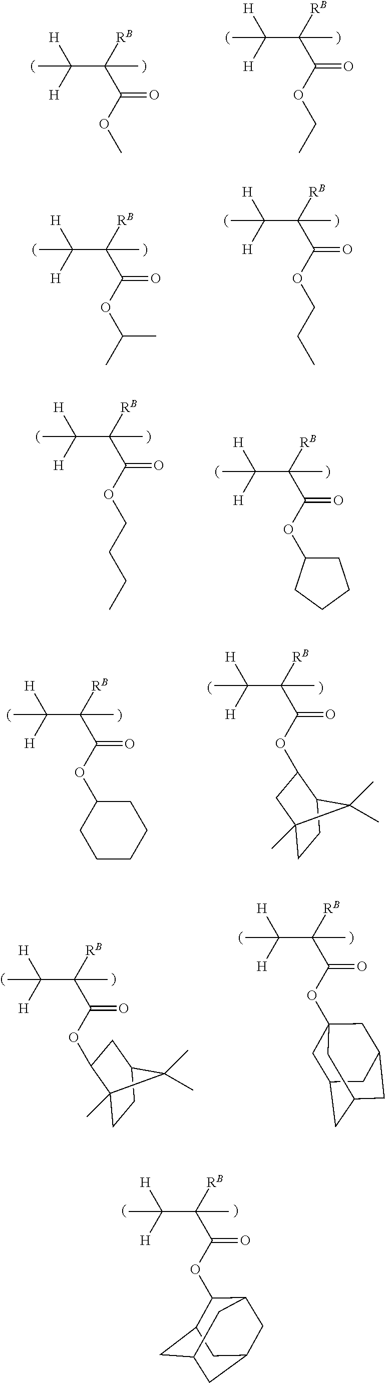

The polymeric surfactant may further contain a repeat unit other than the repeat units having the formulae (7A) to (7E). Examples of the other repeat unit include repeat units obtained from methacrylic acid, α-trifluoromethylacrylic acid derivatives, and the like. The content of the repeat units having the formulae (7A) to (7E) in the polymeric surfactant is preferably 20 mol % or more, more preferably 60 mol % or more, still more preferably 100 mol % of the overall repeat units.

The polymeric surfactant preferably has a Mw of 1,000 to 500,000, and more preferably 3,000 to 100,000. Mw/Mn is preferably 1.0 to 2.0, and more preferably 1.0 to 1.6.

Examples of the method for synthesizing the polymeric surfactant include a method in which a monomer containing a repeat unit having any of the formulae (7A) to (7E) and, if necessary, an unsaturated bond to give another repeat unit is heated and polymerized by adding a radical initiator in an organic solvent. Examples of the organic solvent used in the polymerization include toluene, benzene, THF, diethyl ether, and dioxane. Specific examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

When the polymeric surfactant is synthesized, a known chain transfer agent such as dodecylmercaptan or 2-mercaptoethanol may be used for the purpose of adjusting the molecular weight. In that case, the amount of such a chain transfer agent added is preferably 0.01 to 10 mol % based on the total number of moles of monomers to be polymerized.

When the chemically amplified resist composition of the invention contains the surfactant (F), the content thereof is preferably 0.1 to 50 parts by weight, and more preferably 0.5 to 10 parts by weight per 80 parts by weight of the base polymer (B). When the content of the surfactant (F) is 0.1 parts by weight or more, the receding contact angle between the surface of the resist film and water is sufficiently improved, and when the content of the surfactant is 50 parts by weight or less, the dissolution rate of the surface of the resist film in the developer is low, and the height of the formed fine pattern is sufficiently maintained.

As the surfactant (F), one type may be used alone or two or more types may be combined and used.

(G) Other components

The chemically amplified resist composition of the invention may contain, as another component (G), a compound which is decomposed with an acid to generate another acid (acid amplifier compound), an organic acid derivative, a fluorine-substituted alcohol, a compound having a Mw of 3,000 or less and changes its solubility in a developer by the action of an acid (dissolution inhibitor), or the like. As the acid amplifier compound, reference may be made to compounds described in JP-A 2009-269953 or JP-A 2010-215608. When the acid amplifier compound is contained, the content thereof is preferably 0 to 5 parts by weight, and more preferably 0 to 3 parts by weight per 80 parts by weight of the base polymer (B). If the content is too large, it is difficult to control acid diffusion, and deterioration in resolution and deterioration in pattern shape may occur. As the organic acid derivative, the fluorine-substituted alcohol, and the dissolution inhibitor, reference may be made to compounds described in JP-A 2009-269953 or JP-A 2010-215608.

Pattern forming process

A pattern forming process of the invention includes the steps of forming a resist film on a substrate using the chemically amplified resist composition, exposing the resist film to high-energy radiation, and developing the exposed resist film using a developer.

As the substrate, for example, a substrate for producing an integrated circuit (such as Si, SiO₂, SiN, SiON, TiN, WSi, BPSG, SOG, or an organic antireflective film) or a substrate for producing a mask circuit (such as Cr, CrO, CrON, MoSi₂, or SiO₂) can be used.

The resist film can be formed by, for example, applying the chemically amplified resist composition onto a substrate by a method such as spin coating so as to have a film thickness of preferably 0.05 to 2 μm, and prebaking the chemically amplified resist composition on a hotplate at preferably 60 to 150° C. for 1 to 10 minutes, more preferably at 80 to 140° C. for 1 to 5 minutes.

Examples of the high-energy radiation used for exposure of the resist film include KrF excimer laser light, ArF excimer laser light, EB, and EUV with a wavelength of 3 to 15 nm. When KrF excimer laser light, ArF excimer laser light, or EUV is used, exposure can be performed by using a mask for forming a desired pattern and performing irradiation so that the exposure dose is preferably 1 to 200 mJ/cm² and more preferably 10 to 100 mJ/cm². When EB is used, irradiation is performed using a mask for forming a desired pattern or directly so that the exposure dose is preferably 1 to 300 μC/cm² and more preferably 10 to 200 μC/cm².

The exposure can be performed by an ordinary exposure method, or can be performed by also using an immersion method in which a liquid having a refractive index of 1.0 or more is interposed between the resist film and a projection lens. In this case, a protective film insoluble in water can also be used.

The protective film insoluble in water is used for preventing an eluted material from the resist film and for improving water sliding on the film surface, and roughly includes two types. The first type is an organic solvent-strippable protective film which needs to be stripped with an organic solvent which does not dissolve the resist film before development with an alkaline aqueous solution, and the second type is an alkaline aqueous solution-soluble protective film which is soluble in an alkaline developer and is removed together with the removal of a soluble portion in the resist film. The second type is particularly preferably a material that contains, as a base, a polymer having a 1,1,1,3,3,3-hexafluoro-2-propanol residue insoluble in water and soluble in an alkaline developer, and that is dissolved in an alcohol-based solvent having 4 or more carbon atoms, a C₈-C₁₂ ether-based solvent, or a mixed solvent thereof. The second type can also be a material obtained by dissolving the above-mentioned surfactant insoluble in water and soluble in an alkaline developer in an alcohol-based solvent having 4 or more carbon atoms, a C₈-C₁₂ ether-based solvent, or a mixed solvent thereof.

PEB may be performed after the exposure. PEB can be performed, for example, by heating on a hotplate at preferably 60 to 150° C. for 1 to 5 minutes, more preferably at 80 to 140° C. for 1 to 3 minutes.

In the development, for example, a developer of an alkaline aqueous solution of, for example, tetramethylammonium hydroxide (TMAH) at preferably 0.1 to 5 mass %, more preferably 2 to 3 mass % is used, and the development is performed for preferably 0.1 to 3 minutes, more preferably 0.5 to 2 minutes by a conventional method such as a dip method, a puddle method, or a spray method to dissolve the exposed portion and form a desired pattern on the substrate.

After the resist film is formed, the acid generator or the like may be extracted from the film surface by performing rinsing with pure water, or particles may be washed off, or rinsing for removing water remaining on the film after exposure may be performed.

Further, a double patterning method may be used for pattern formation. Examples of the double patterning method include a trench method in which an underlayer of a 1:3 trench pattern is processed by first exposure and etching, and a 1:3 trench pattern is formed by second exposure with the position shifted to form a 1:1 pattern, and a line method in which a first underlayer of a 1:3 isolated left pattern is processed by first exposure and etching, and a second underlayer having the formed 1:3 isolated left pattern under the first underlayer is processed by second exposure with the position shifted to form a 1:1 pattern with a half pitch.

In the pattern forming process of the invention, a method of negative tone development in which an unexposed portion is dissolved using an organic solvent as a developer instead of the alkaline aqueous solution may be used.

In the organic solvent development, as the developer, 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, or the like can be used. One type of these organic solvents may be used alone, or two or more types of organic solvents may be mixed and used.

### EXAMPLES

Hereinafter, the invention is specifically described with showing Synthesis Examples, Examples, and Comparative Examples, but the invention is not limited to the following Examples. The apparatus used is as follows.

MALDI TOF-MS: S3000 manufactured by JEOL Ltd.

[1] Synthesis of onium salts

### Example 1-1

Synthesis of onium salt SQ-1

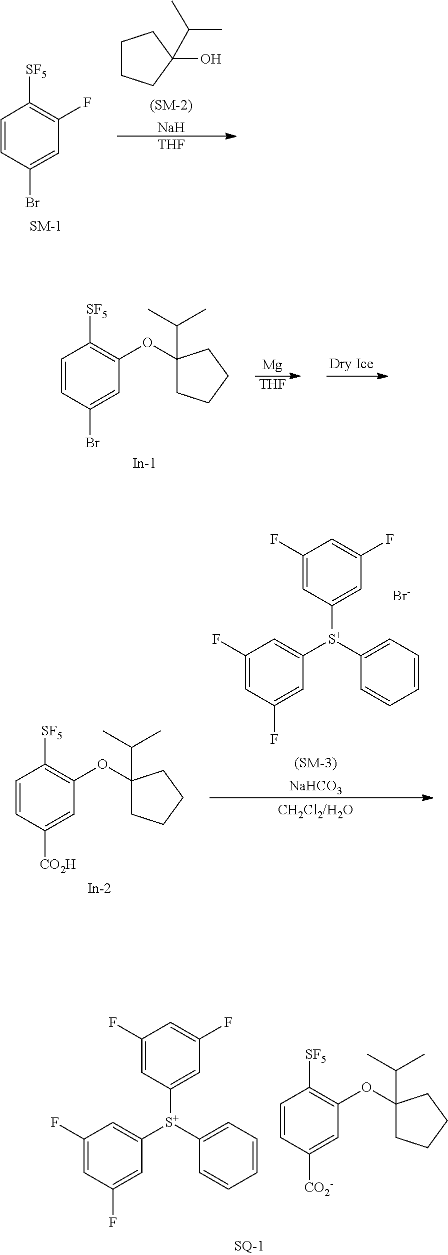

### (1) Synthesis of Intermediate In-1

Under a nitrogen atmosphere, sodium hydride (purity: 55 mass %, 4.6 g) was suspended in THF (50 mL), and a solution containing a raw material SM-1 (17.6 g) and THF (30 mL) was added dropwise thereto. After the dropwise addition, heating reflux was performed for 4 hours to prepare a metal alkoxide. Thereafter, the raw material SM-1 (30.1 g) was added dropwise thereto, and heating reflux and aging were performed for 18 hours. The reaction solution was cooled in an ice bath and the reaction was stopped with water (100 mL). The target product was extracted twice with a mixed solvent of toluene (100 mL) and hexane (100 mL), subjected to a normal aqueous work-up, the solvent was distilled off, followed by distillation purification to obtain 29.0 g of an intermediate In-1 as a colorless oily matter (yield: 71%).

(2) Synthesis of Intermediate in-2

Under a nitrogen atmosphere, a Grignard reagent was prepared from metallic magnesium (1.8 g), the intermediate In-1 (29.0 g) and THF (100 mL). This Grignard reagent was added dropwise to a suspension of dry ice (100 g) in THF (100 mL). After the dropwise addition, the mixture was aged until the dry ice sublimated. After the aging, 5 mass % hydrochloric acid (100 g) was added dropwise to stop the reaction while maintaining the reaction solution at 10° C. or lower. Thereafter, the target product was extracted with ethyl acetate (300 mL), subjected to a normal aqueous work-up, the solvent was distilled off, followed by recrystallization with hexane to obtain an intermediate In-2 as white crystals (amount: 16.7 g, yield: 63%).

### (3) Synthesis of Onium Salt SQ-1

Under a nitrogen atmosphere, the intermediate In-2 (3.7 g) and sodium hydrogen carbonate (0.9 g) were added to water (40 g) in a reaction vessel, and the mixture was stirred at a temperature of 50° C. in the reaction vessel for 2 hours. Thereafter, the reaction solution was cooled to room temperature, a raw material SM-2 (4.6 g) and methylene chloride (50 g) were added thereto, and the mixture was stirred for 20 minutes. The reaction solution was separated, and the organic layer was taken out, then subjected to a normal aqueous work-up, and the solvent was distilled off to obtain 5.9 g of an onium salt SQ-1 as a colorless oily matter (yield: 83%).

The results of TOF-MS of the onium salt SQ-1 are shown below.

### MALDI TOF-MS:

POSITIVE M⁺335 (corresponding to C₁₈H₁₁F₄S⁺)

NEGATIVE M-373 (corresponding to C₁₅H₁₈F₅O₃S⁻)

### Examples 1-2 to 1-9

### Synthesis of Onium Salts SQ-2 to SQ-9

Various onium salts were synthesized by various organic synthesis reactions. The structures of the onium salts used in the chemically amplified resist compositions are shown below.

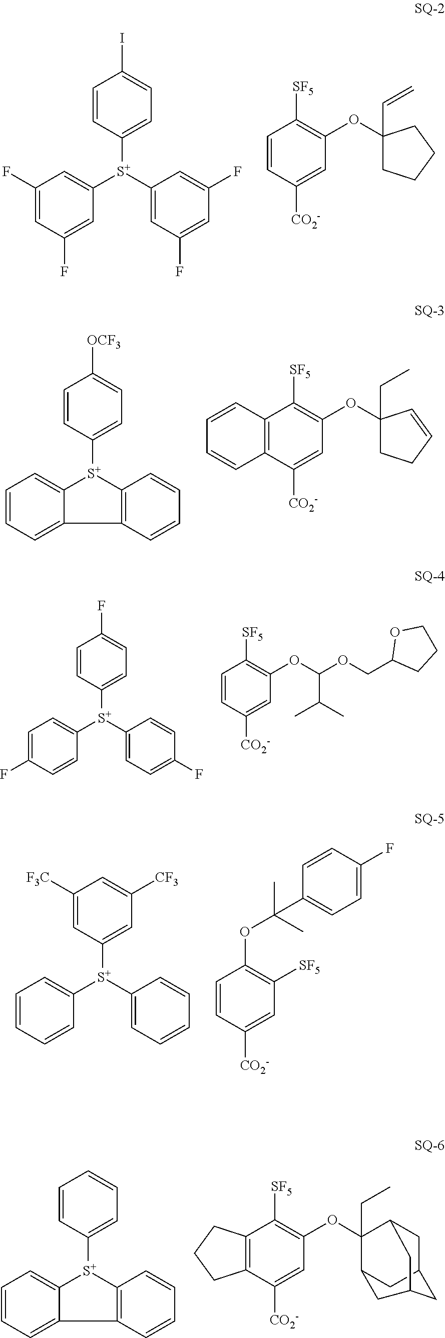

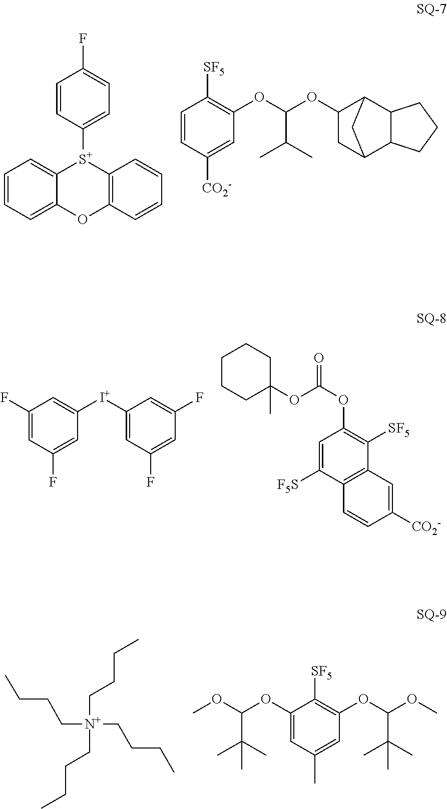

### Synthesis Example

### Synthesis of Base Polymers P-1 to P-6

Base polymers P-1 to P-6 having the composition shown below were synthesized by combining monomers, performing a copolymerization reaction in MEK as a solvent, pouring the reaction solution to hexane for precipitation, washing the precipitated solid with hexane, followed by isolation and drying. The obtained base polymers were analyzed for the composition by 1H-NMR, and for Mw and Mw/Mn by GPC (solvent: THF, standard: polystyrene).

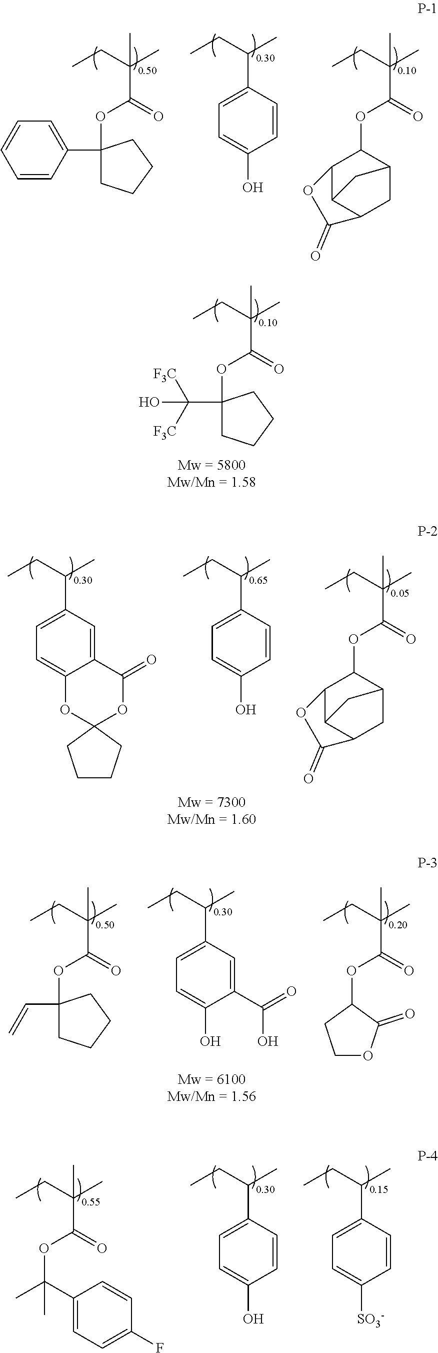

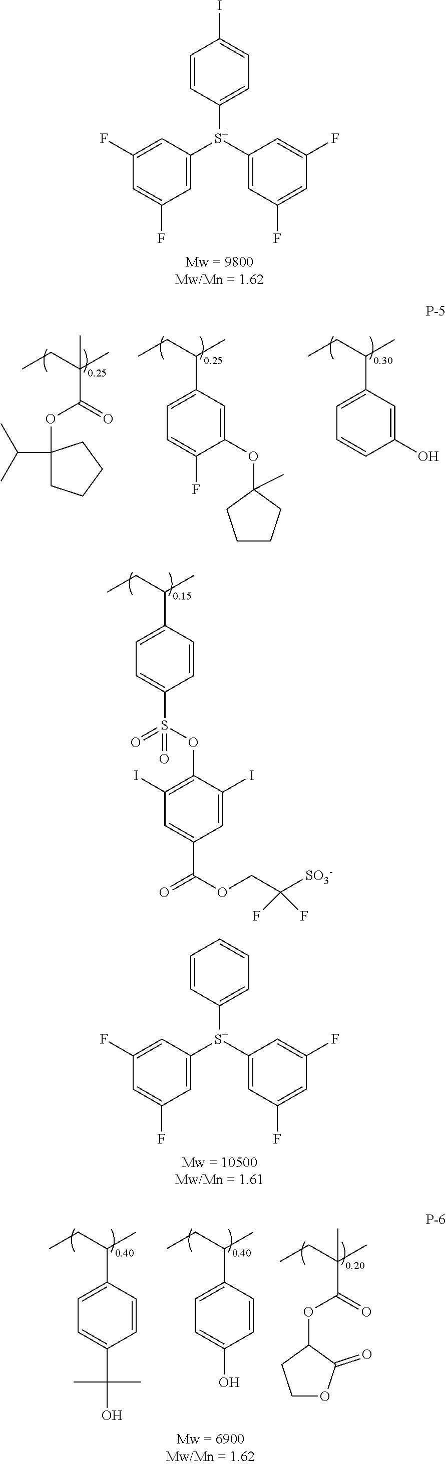

[2] Preparation of chemically amplified resist compositions

### Examples 2-1 to 2-45 and Comparative Examples 1-1 to 1-25

Chemically amplified resist compositions (R-1 to R-45, CR-1 to CR-25) were prepared by dissolving an onium salt (SQ-1 to SQ-9) of the invention, a comparative quencher (SQ-A to SQ-D), a blend quencher (bQ-1, bQ-2), a photoacid generator (PAG-X, PAG-Y), and a base polymer (P-1 to P-6) in a solvent containing 0.01 mass % of a surfactant A (Omnova) according to the formulation shown in Tables 1 to 3 to prepare solutions, and filtering the solutions through a Teflon® filter with a pore size of 0.2 μm.

TABLE 1

| | | | Base | | Photoacid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Example | 2-1 | R-1 | P-1 (80) | SQ-1 (8.0) | PAG-X (24) | PGMEA (2250) | EL (2800) | DAA (550) |
| | 2-2 | R-2 | P-1 (80) | SQ-2 (8.0) | PAG-X (24) | PGMEA (2250) | EL (2800) | DAA (550) |
| | 2-3 | R-3 | P-1 (80) | SQ-3 (8.2) | PAG-X (24) | PGMEA (2250) | EL (2800) | DAA (550) |
| | 2-4 | R-4 | P-1 (80) | SQ-4 (8.0) | PAG-X (24) | PGMEA (2250) | EL (2800) | DAA (550) |
| | 2-5 | R-5 | P-1 (80) | SQ-5 (8.2) | PAG-X (24) | PGMEA (2250) | EL (2800) | DAA (550) |
| | 2-6 | R-6 | P-1 (80) | SQ-6 (8.0) | PAG-X (24) | PGMEA (2250) | EL (2800) | DAA (550) |
| | 2-7 | R-7 | P-1 (80) | SQ-7 (7.6) | PAG-X (24) | PGMEA (2250) | EL (2800) | DAA (550) |
| | 2-8 | R-8 | P-1 (80) | SQ-8 (8.0) | PAG-X (24) | PGMEA (2250) | EL (2800) | DAA (550) |
| | 2-9 | R-9 | P-1 (80) | SQ-9 (4.0) | PAG-X (24) | PGMEA (2250) | EL (2800) | DAA (550) |
| | | | | bQ-1 (4.0) | | | | |
| | 2-10 | R-10 | P-1 (80) | SQ-1 (8.0) | PAG-Y (24) | PGMEA (2250) | EL (2800) | DAA (550) |
| | 2-11 | R-11 | P-1 (80) | SQ-2 (8.2) | PAG-Y (22) | PGMEA (2250) | EL (2800) | DAA (550) |
| | 2-12 | R-12 | P-1 (80) | SQ-3 (7.6) | PAG-Y (24) | PGMEA (2250) | EL (2800) | DAA (550) |
| | 2-13 | R-13 | P-1 (80) | SQ-4 (8.0) | PAG-Y (24) | PGMEA (2250) | EL (2800) | DAA (550) |
| | 2-14 | R-14 | P-1 (80) | SQ-5 (8.2) | PAG-Y (22) | PGMEA (2250) | EL (2800) | DAA (550) |
| | 2-15 | R-15 | P-1 (80) | SQ-6 (8.0) | PAG-Y (24) | PGMEA (2250) | EL (2800) | DAA (550) |
| | 2-16 | R-16 | P-1 (80) | SQ-7 (7.8) | PAG-Y (24) | PGMEA (2250) | EL (2800) | DAA (550) |
| | 2-17 | R-17 | P-1 (80) | SQ-8 (8.0) | PAG-Y (24) | PGMEA (2250) | EL (2800) | DAA (550) |
| | 2-18 | R-18 | P-1 (80) | SQ-9 (5.0) | PAG-Y (24) | PGMEA (2250) | EL (2800) | DAA (550) |
| | | | | bQ-1 (2.6) | | | | |
| | 2-19 | R-19 | P-1 (80) | SQ-1 (4.0) | PAG-X (22) | PGMEA (2250) | EL (2800) | DAA (550) |
| | | | | bQ-1 (3.6) | | | | |
| | 2-20 | R-20 | P-1 (80) | SQ-2 (4.0) | PAG-Y (24) | PGMEA (2250) | EL (2800) | DAA (550) |
| | | | | bQ-2 (3.6) | | | | |
| | 2-21 | R-21 | P-2 (80) | SQ-1 (8.0) | PAG-X (24) | PGMEA (2250) | EL (2800) | DAA (550) |
| | 2-22 | R-22 | P-2 (80) | SQ-3 (8.2) | PAG-Y (24) | PGMEA (2250) | EL (2800) | DAA (550) |
| | 2-23 | R-23 | P-2 (80) | SQ-4 (8.0) | PAG-X (22) | PGMEA (2250) | EL (2800) | DAA (550) |
| | 2-24 | R-24 | P-2 (80) | SQ-6 (7.6) | PAG-X (22) | PGMEA (2250) | EL (2800) | DAA (550) |
| | 2-25 | R-25 | P-2 (80) | SQ-8 (8.2) | PAG-Y (24) | PGMEA (2250) | EL (2800) | DAA (550) |

TABLE 2

| | | | Base | | Photoacid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Example | 2-26 | R-26 | P-3 (80) | SQ-1 (8.0) | PAG-Y (24) | PGMEA (2250) | EL (2800) | DAA (550) |
| | 2-27 | R-27 | P-3 (80) | SQ-2 (7.8) | PAG-X (24) | PGMEA (2250) | EL (2800) | DAA (550) |
| | 2-28 | R-28 | P-3 (80) | SQ-5 (8.2) | PAG-Y (24) | PGMEA (2250) | EL (2800) | DAA (550) |
| | 2-29 | R-29 | P-3 (80) | SQ-7 (8.0) | PAG-X (20) | PGMEA (2250) | EL (2800) | DAA (550) |
| | 2-30 | R-30 | P-3 (80) | SQ-3 (4.0) | PAG-Y (24) | PGMEA (2250) | EL (2800) | DAA (550) |
| | | | | bQ-1 (4.0) | | | | |
| | 2-31 | R-31 | P-4 (80) | SQ-1 (8.0) | — | PGMEA (2250) | EL (2800) | DAA (550) |
| | 2-32 | R-32 | P-4 (80) | SQ-3 (8.0) | — | PGMEA (2250) | EL (2800) | DAA (550) |
| | 2-33 | R-33 | P-4 (80) | SQ-4 (8.2) | — | PGMEA (2250) | EL (2800) | DAA (550) |
| | 2-34 | R-34 | P-4 (80) | SQ-6 (7.6) | — | PGMEA (2250) | EL (2800) | DAA (550) |
| | 2-35 | R-35 | P-4 (80) | SQ-8 (8.0) | PAG-Y (10) | PGMEA (2250) | EL (2800) | DAA (550) |
| | 2-36 | R-36 | P-5 (80) | SQ-1 (8.0) | — | PGMEA (2250) | EL (2800) | DAA (550) |
| | 2-37 | R-37 | P-5 (80) | SQ-2 (8.2) | — | PGMEA (2250) | EL (2800) | DAA (550) |
| | 2-38 | R-38 | P-5 (80) | SQ-5 (7.8) | — | PGMEA (2250) | EL (2800) | DAA (550) |
| | 2-39 | R-39 | P-5 (80) | SQ-6 (8.0) | PAG-X (8) | PGMEA (2250) | EL (2800) | DAA (550) |
| | 2-40 | R-40 | P-5 (80) | SQ-2 (4.0) | — | PGMEA (2250) | EL (2800) | DAA (550) |
| | | | | bQ-2 (4.0) | | | | |
| | 2-41 | R-41 | P-6 (80) | SQ-1 (8.0) | PAG-X (24) | PGMEA (2250) | EL (2800) | DAA (550) |
| | 2-42 | R-42 | P-6 (80) | SQ-3 (7.6) | PAG-Y (22) | PGMEA (2250) | EL (2800) | DAA (550) |
| | 2-43 | R-43 | P-6 (80) | SQ-5 (8.2) | PAG-X (24) | PGMEA (2250) | EL (2800) | DAA (550) |
| | 2-44 | R-44 | P-6 (80) | SQ-7 (8.0) | PAG-Y (22) | PGMEA (2250) | EL (2800) | DAA (550) |
| | 2-45 | R-45 | P-6 (80) | SQ-4 (4.8) | PAG-X (24) | PGMEA (2250) | EL (2800) | DAA (550) |
| | | | | bQ-2 (3.6) | | | | |

TABLE 3

| | | Resist | Base | | Photoacid | | | |
|---|---|---|---|---|---|---|---|---|
| | | compo- | polymer | Quencher | generator | Solvent 1 | Solvent 2 | Solvent 3 |
| | | sition | (pbw) | (pbw) | (pbw) | (pbw) | (pbw) | (pbw) |
| Com- | 1-1 | CR-1 | P-1 (80) | SQ-A (8.0) | PAG-X (24) | PGMEA (2250) | EL (2800) | DAA (550) |
| parative | 1-2 | CR-2 | P-1 (80) | SQ-B (8.2) | PAG-X (24) | PGMEA (2250) | EL (2800) | DAA (550) |
| Example | 1-3 | CR-3 | P-1 (80) | SQ-C (8.0) | PAG-X (24) | PGMEA (2250) | EL (2800) | DAA (550) |
| | 1-4 | CR-4 | P-1 (80) | SQ-D (7.8) | PAG-X (24) | PGMEA (2250) | EL (2800) | DAA (550) |
| | 1-5 | CR-5 | P-1 (80) | SQ-A (8.0) | PAG-Y (24) | PGMEA (2250) | EL (2800) | DAA (550) |
| | 1-6 | CR-6 | P-1 (80) | SQ-B (7.6) | PAG-Y (24) | PGMEA (2250) | EL (2800) | DAA (550) |
| | 1-7 | CR-7 | P-1 (80) | SQ-C (8.2) | PAG-Y (24) | PGMEA (2250) | EL (2800) | DAA (550) |
| | 1-8 | CR-8 | P-1 (80) | SQ-D (7.8) | PAG-Y (24) | PGMEA (2250) | EL (2800) | DAA (550) |
| | 1-9 | CR-9 | P-1 (80) | SQ-A (4.0) | PAG-X (22) | PGMEA (2250) | EL (2800) | DAA (550) |
| | | | | bQ-1 (3.6) | | | | |
| | 1-10 | CR-10 | P-1 (80) | SQ-B (4.0) | PAG-Y (24) | PGMEA (2250) | EL (2800) | DAA (550) |
| | | | | bQ-2 (3.6) | | | | |
| | 1-11 | CR-11 | P-2 (80) | SQ-A (8.0) | PAG-X (24) | PGMEA (2250) | EL (2800) | DAA (550) |
| | 1-12 | CR-12 | P-2 (80) | SQ-B (8.2) | PAG-Y (24) | PGMEA (2250) | EL (2800) | DAA (550) |
| | 1-13 | CR-13 | P-2 (80) | SQ-C (8.0) | PAG-X (22) | PGMEA (2250) | EL (2800) | DAA (550) |
| | 1-14 | CR-14 | P-3 (80) | SQ-A (8.2) | PAG-Y (24) | PGMEA (2250) | EL (2800) | DAA (550) |
| | 1-15 | CR-15 | P-3 (80) | SQ-C (8.0) | PAG-X (20) | PGMEA (2250) | EL (2800) | DAA (550) |
| | 1-16 | CR-16 | P-3 (80) | SQ-C (4.0) | PAG-Y (24) | PGMEA (2250) | EL (2800) | DAA (550) |
| | | | | bQ-1 (4.0) | | | | |
| | 1-17 | CR-17 | P-4 (80) | SQ-A (8.2) | — | PGMEA (2250) | EL (2800) | DAA (550) |
| | 1-18 | CR-18 | P-4 (80) | SQ-C (7.6) | — | PGMEA (2250) | EL (2800) | DAA (550) |
| | 1-19 | CR-19 | P-4 (80) | SQ-D (8.0) | PAG-Y (10) | PGMEA (2250) | EL (2800) | DAA (550) |
| | 1-20 | CR-20 | P-5 (80) | SQ-A (8.2) | — | PGMEA (2250) | EL (2800) | DAA (550) |
| | 1-21 | CR-21 | P-5 (80) | SQ-B (7.8) | — | PGMEA (2250) | EL (2800) | DAA (550) |
| | 1-22 | CR-22 | P-5 (80) | SQ-A (8.0) | PAG-X (8) | PGMEA (2250) | EL (2800) | DAA (550) |
| | 1-23 | CR-23 | P-6 (80) | SQ-A (8.2) | PAG-X (24) | PGMEA (2250) | EL (2800) | DAA (550) |
| | 1-24 | CR-24 | P-6 (80) | SQ-B (8.0) | PAG-Y (22) | PGMEA (2250) | EL (2800) | DAA (550) |
| | 1-25 | CR-25 | P-6 (80) | SQ-D (4.8) | PAG-X (24) | PGMEA (2250) | EL (2800) | DAA (550) |
| | | | | bQ-2 (3.6) | | | | |

In Tables 1 to 3, solvents, photoacid generators PAG-X and PAG-Y, comparative quenchers SQ-A to SQ-D, and blend quenchers bQ-1 and bQ-2 are as follows.

### Solvent:

PGMEA (propylene glycol monomethyl ether acctate)

EL (ethyl lactate)

DAA (diacetone alcohol)

### Photoacid Generator: PAG-X and PAG-Y

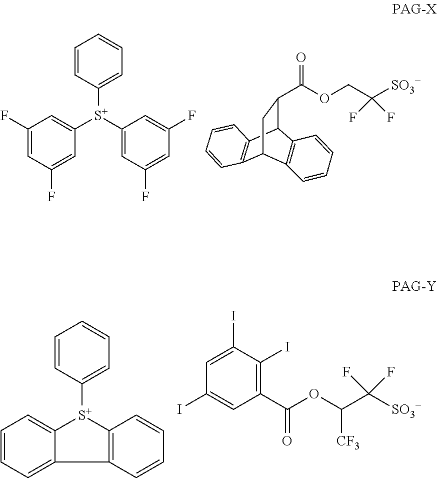

### Comparative Quencher: SQ-A to SQ-D

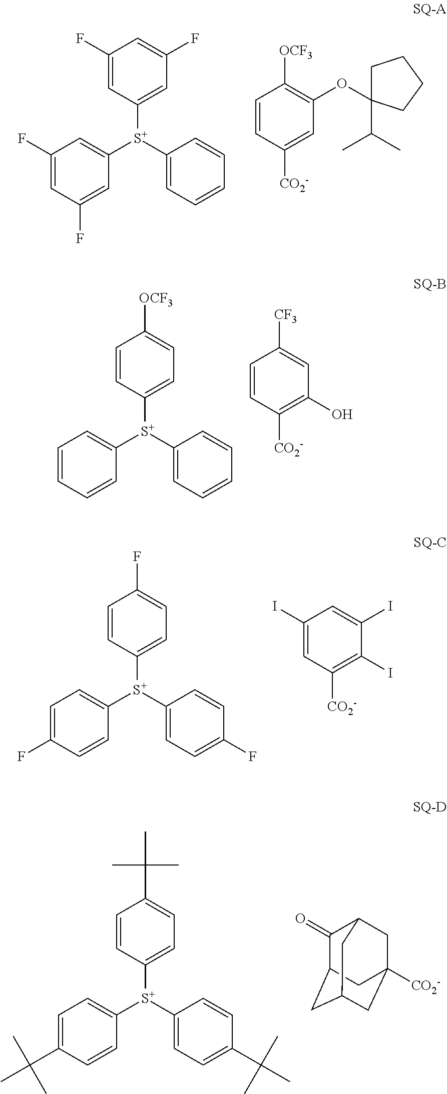

Blend quencher: bQ-1 and bQ-2

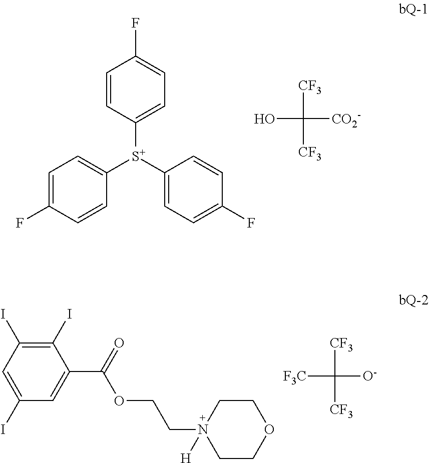

### [3] EUV Lithography Test 1

### Examples 3-1 to 3-45 and Comparative Examples 2-1 to 2-25

Each of the chemically amplified resist compositions (R-1 to R-45, CR-1 to CR-25) shown in Tables 1 to 3 was applied by spin coating onto a Si substrate having a silicon-containing spin-on-hard mask SHB-A940 manufactured by Shin-Etsu Chemical Co., Ltd. (silicon content: 43 mass %) formed to a film thickness of 20 nm and prebaked on a hotplate at 100° C. for 60 seconds to prepare a resist film having a film thickness of 50 nm. The resist film was exposed using an EUV scanner NXE3400 (NA 0.33, 0.9/0.6, dipole illumination) manufactured by ASML Holding N.V. through a mask bearing an LS pattern having a size of 18 nm and a pitch of 36 nm on-wafer size while changing the exposure dose and focus (exposure dose pitch: 1 mJ/cm², focus pitch: 0.020 μm), and after the exposure, PEB was performed at a temperature shown in Tables 4 to 6 for 60 seconds. This was followed by puddle development in a 2.38 mass % TMAH aqueous solution for 30 seconds, rinsing with a surfactant-containing rinse material, and spin drying to obtain a positive pattern in Examples 3-1 to 3-40 and Comparative Examples 2-1 to 2-22. In Examples 3-41 to 3-45 and Comparative Examples 2-23 to 2-25, a negative pattern was obtained.

The obtained LS pattern was observed with a length measuring SEM (CG6300) manufactured by Hitachi High-Technologies Corporation, and the sensitivity, EL, LWR, depth of focus (DOF), and collapse limit were evaluated according to the following methods. The results are shown in Tables 4 to 6.

### [Evaluation of Sensitivity]

The optimum exposure dose Eop (mJ/cm²) which provided an LS pattern with a line width of 18 nm and a pitch of 36 nm was determined and reported as sensitivity. A smaller value indicates a higher sensitivity.

### [Evaluation of EL]

EL (unit: %) was determined from the exposure dose which provides an LS pattern with a space width of 18 nm±10% (16.2 to 19.8 nm) according to the following formula. A larger value indicates better performance.

$$EL ( \% ) = ( \vert {E}_{1} - {E}_{2} \vert / Eop ) \times 100$$

E₁: an optimum exposure dose which provides an LS pattern with a line width of 16.2 nm and a pitch of 36 nm

E₂: an optimum exposure dose which provides an LS pattern with a line width of 19.8 nm and a pitch of 36 nm

Eop: an optimum exposure dose which provides an LS pattern with a line width of 18 nm and a pitch of 36 nm

### [Evaluation of LWR]

In the LS pattern formed by exposure at Eop, the line width was measured at 10 longitudinally spaced apart points, from which a 3-fold value (30) of the standard deviation (σ) was determined as LWR. A smaller value indicates a pattern having small roughness and uniform line width.

### [Evaluation of DOF]

As an index of the depth of focus, a range of focus which provided an LS pattern with a size of 18 nm±10% (16.2 to 19.8 nm) was determined. A greater value indicates a wider depth of focus.

### [Evaluation of Collapse Limit of Line Pattern]

The line size of the LS pattern at each exposure dose with the optimum focus was measured at 10 longitudinally spaced apart points. A narrowest line size obtained without collapse was determined as a collapse limit size. A smaller value indicates better collapse limit.

TABLE 4

| | | | | Optimum | | | | |
|---|---|---|---|---|---|---|---|---|
| | | | PEB | exposure | | | | Collapse |
| | | Resist | temp. | dose | EL | LWR | DOF | limit |
| | | composition | (° C.) | (mJ/cm²) | (%) | (nm) | (nm) | (nm) |
| Example | 3-1 | R-1 | 95 | 32 | 18 | 2.2 | 120 | 10.7 |
| | 3-2 | R-2 | 100 | 32 | 19 | 2.5 | 110 | 10.8 |
| | 3-3 | R-3 | 100 | 33 | 17 | 2.3 | 110 | 11.1 |
| | 3-4 | R-4 | 95 | 33 | 18 | 2.3 | 110 | 10.9 |
| | 3-5 | R-5 | 105 | 34 | 17 | 2.4 | 110 | 11.1 |
| | 3-6 | R-6 | 100 | 3 | 18 | 2.3 | 100 | 11.3 |
| | 3-7 | R-7 | 95 | 34 | 18 | 2.3 | 110 | 10.8 |
| | 3-8 | R-8 | 95 | 32 | 17 | 2.5 | 120 | 10.7 |
| | 3-9 | R-9 | 100 | 32 | 16 | 2.2 | 110 | 11.1 |
| | 3-10 | R-10 | 100 | 33 | 19 | 2.5 | 110 | 11.0 |
| | 3-11 | R-11 | 100 | 35 | 17 | 2.2 | 110 | 11.1 |
| | 3-12 | R-12 | 95 | 34 | 18 | 2.5 | 100 | 11.4 |
| | 3-13 | R-13 | 105 | 33 | 17 | 2.4 | 110 | 10.8 |
| | 3-14 | R-14 | 100 | 32 | 18 | 2.3 | 120 | 10.6 |
| | 3-15 | R-15 | 95 | 34 | 18 | 2.3 | 110 | 10.6 |
| | 3-16 | R-16 | 95 | 34 | 17 | 2.2 | 110 | 10.8 |
| | 3-17 | R-17 | 100 | 32 | 17 | 2.4 | 120 | 11.2 |
| | 3-18 | R-18 | 95 | 34 | 16 | 2.5 | 120 | 11.1 |
| | 3-19 | R-19 | 95 | 35 | 17 | 2.4 | 110 | 11.3 |
| | 3-20 | R-20 | 100 | 32 | 18 | 2.5 | 120 | 11.3 |
| | 3-21 | R-21 | 100 | 32 | 17 | 2.5 | 110 | 11.2 |
| | 3-22 | R-22 | 100 | 34 | 19 | 2.3 | 120 | 11.5 |
| | 3-23 | R-23 | 95 | 35 | 18 | 2.2 | 100 | 11.1 |
| | 3-24 | R-24 | 95 | 33 | 17 | 2.4 | 110 | 10.7 |
| | 3-25 | R-25 | 100 | 35 | 17 | 2.3 | 100 | 11.2 |

TABLE 5

| | | | | Optimum | | | | |
|---|---|---|---|---|---|---|---|---|
| | | | PEB | exposure | | | | Collapse |
| | | Resist | temp. | dose | EL | LWR | DOF | limit |
| | | composition | (° C.) | (mJ/cm²) | (%) | (nm) | (nm) | (nm) |
| Example | 3-26 | R-26 | 100 | 33 | 17 | 2.4 | 110 | 11.2 |
| | 3-27 | R-27 | 100 | 32 | 18 | 2.5 | 120 | 10.7 |
| | 3-28 | R-28 | 95 | 32 | 17 | 2.3 | 110 | 11.4 |
| | 3-29 | R-29 | 95 | 35 | 19 | 2.5 | 100 | 10.8 |
| | 3-30 | R-30 | 100 | 34 | 18 | 2.4 | 120 | 10.8 |
| | 3-31 | R-31 | 100 | 33 | 17 | 2.4 | 110 | 11.1 |
| | 3-32 | R-32 | 100 | 35 | 18 | 2.3 | 110 | 11.2 |
| | 3-33 | R-33 | 95 | 32 | 18 | 2.5 | 100 | 10.9 |
| | 3-34 | R-34 | 95 | 32 | 17 | 2.3 | 120 | 10.7 |
| | 3-35 | R-35 | 100 | 34 | 17 | 2.3 | 110 | 10.7 |
| | 3-36 | R-36 | 100 | 33 | 17 | 2.4 | 110 | 10.9 |
| | 3-37 | R-37 | 100 | 35 | 18 | 2.5 | 120 | 10.6 |
| | 3-38 | R-38 | 95 | 33 | 17 | 2.2 | 110 | 11.2 |
| | 3-39 | R-39 | 95 | 32 | 19 | 2.5 | 100 | 11.1 |
| | 3-40 | R-40 | 100 | 33 | 18 | 2.3 | 120 | 10.9 |
| | 3-41 | R-41 | 100 | 33 | 18 | 2.4 | 110 | 10.9 |
| | 3-42 | R-42 | 100 | 34 | 18 | 2.3 | 110 | 10.7 |
| | 3-43 | R-43 | 100 | 33 | 17 | 2.5 | 120 | 10.7 |
| | 3-44 | R-44 | 100 | 35 | 18 | 2.2 | 110 | 10.9 |
| | 3-45 | R-45 | 100 | 33 | 17 | 2.5 | 110 | 11.1 |

TABLE 6

| | | | | Optimum | | | | |
|---|---|---|---|---|---|---|---|---|
| | | | PEB | exposure | | | | Collapse |
| | | Resist | temp. | dose | EL | LWR | DOF | limit |
| | | composition | (° C.) | (mJ/cm²) | (%) | (nm) | (nm) | (nm) |
| Comparative | 2-1 | CR-1 | 95 | 36 | 14 | 2.7 | 100 | 11.3 |
| Example | 2-2 | CR-2 | 100 | 38 | 13 | 2.9 | 80 | 11.9 |
| | 2-3 | CR-3 | 100 | 38 | 14 | 3.0 | 90 | 12.4 |
| | 2-4 | CR-4 | 100 | 39 | 12 | 3.2 | 70 | 12.8 |
| | 2-5 | CR-5 | 95 | 38 | 15 | 2.8 | 90 | 11.5 |
| | 2-6 | CR-6 | 100 | 37 | 14 | 2.9 | 80 | 11.8 |
| | 2-7 | CR-7 | 100 | 38 | 13 | 2.9 | 70 | 11.7 |
| | 2-8 | CR-8 | 100 | 39 | 12 | 3.1 | 70 | 12.6 |
| | 2-9 | CR-9 | 95 | 40 | 14 | 2.9 | 90 | 12.4 |
| | 2-10 | CR-10 | 100 | 38 | 13 | 2.8 | 80 | 11.9 |
| | 2-11 | CR-11 | 100 | 39 | 14 | 2.9 | 90 | 12.3 |
| | 2-12 | CR-12 | 100 | 40 | 15 | 3.0 | 80 | 12.4 |
| | 2-13 | CR-13 | 95 | 38 | 12 | 3.1 | 90 | 12.2 |
| | 2-14 | CR-14 | 105 | 36 | 13 | 3.2 | 100 | 11.9 |
| | 2-15 | CR-15 | 100 | 38 | 14 | 2.9 | 80 | 11.8 |
| | 2-16 | CR-16 | 95 | 39 | 13 | 2.7 | 80 | 11.7 |
| | 2-17 | CR-17 | 95 | 38 | 13 | 2.7 | 90 | 12.2 |
| | 2-18 | CR-18 | 100 | 38 | 15 | 2.8 | 70 | 12.3 |
| | 2-19 | CR-19 | 95 | 40 | 14 | 3.1 | 80 | 11.7 |
| | 2-20 | CR-20 | 95 | 38 | 14 | 2.9 | 90 | 12.2 |
| | 2-21 | CR-21 | 100 | 37 | 13 | 2.8 | 70 | 12.4 |
| | 2-22 | CR-22 | 100 | 38 | 12 | 3.0 | 80 | 11.9 |
| | 2-23 | CR-23 | 100 | 37 | 14 | 2.2 | 90 | 12.0 |
| | 2-24 | CR-24 | 100 | 39 | 14 | 2.9 | 80 | 12.3 |
| | 2-25 | CR-25 | 95 | 40 | 12 | 2.8 | 70 | 11.8 |

From the results shown in Tables 4 to 6, it was found that the chemically amplified resist composition containing the quencher of the invention is excellent in EL, LWR and DOF with good sensitivity in both positive and negative tones. In addition, it was verified that the value of the collapse limit was small and the pattern was resistant to collapse even in fine pattern formation.

[4] EUV lithography test 2

### Examples 4-1 to 4-45 and Comparative Examples 3-1 to 3-25

Each of the chemically amplified resist compositions (R-1 to R-45, CR-1 to CR-25) shown in Tables 1 to 3 was applied by spin coating onto a Si substrate having a silicon-containing spin-on-hard mask SHB-A940 manufactured by Shin-Etsu Chemical Co., Ltd. (silicon content: 43 mass %) formed to a film thickness of 20 nm and prebaked on a hotplate at 100° C. for 60 seconds to prepare a resist film having a film thickness of 60 nm. This resist film was exposed using an EUV scanner NXE3400 (NA 0.33, σ 0.9/0.6, quadrupole illumination, a mask bearing a hole pattern having a pitch of 44 nm±20% bias on-wafer size) manufactured by ASML Holding N.V., and PEB was performed at a temperature shown in Table 7 to 9 for 60 seconds using a hotplate, and development was performed with a 2.38 mass % aqueous TMAH solution for 30 seconds to form a hole pattern with a size of 22 nm in Examples 4-1 to 4-40 and Comparative Examples 3-1 to 3-22, and a dot pattern with a size of 22 nm in Examples 4-41 to 4-45 and Comparative Examples 3-23 to 3-25.

An exposure dose when a hole or dot with a size of 22 nm was formed was measured using a length measuring SEM (CG6300) manufactured by Hitachi High-Technologies Corporation to determine the exposure dose as sensitivity, and the sizes of 50 holes or dots at this time were measured, and a 3-fold value (3σ) of the standard deviation (σ) calculated from the results was determined as CDU. The results are shown in Tables 7 to 9.

TABLE 7

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Example | 4-1 | R-1 | 90 | 22 | 2.3 |
| | 4-2 | R-2 | 95 | 23 | 2.2 |
| | 4-3 | R-3 | 95 | 24 | 2.4 |
| | 4-4 | R-4 | 90 | 25 | 2.5 |
| | 4-5 | R-5 | 90 | 23 | 2.2 |
| | 4-6 | R-6 | 95 | 22 | 2.4 |
| | 4-7 | R-7 | 90 | 22 | 2.4 |
| | 4-8 | R-8 | 95 | 24 | 2.2 |
| | 4-9 | R-9 | 95 | 23 | 2.2 |
| | 4-10 | R-10 | 90 | 24 | 2.5 |
| | 4-11 | R-11 | 95 | 23 | 2.4 |
| | 4-12 | R-12 | 95 | 25 | 2.3 |
| | 4-13 | R-13 | 90 | 23 | 2.3 |
| | 4-14 | R-14 | 90 | 22 | 2.4 |
| | 4-15 | R-15 | 90 | 24 | 2.3 |
| | 4-16 | R-16 | 85 | 25 | 2.5 |
| | 4-17 | R-17 | 95 | 25 | 2.3 |
| | 4-18 | R-18 | 95 | 23 | 2.4 |
| | 4-19 | R-19 | 95 | 22 | 2.2 |
| | 4-20 | R-20 | 95 | 25 | 2.4 |
| | 4-21 | R-21 | 95 | 22 | 2.4 |
| | 4-22 | R-22 | 95 | 22 | 2.3 |
| | 4-23 | R-23 | 95 | 25 | 2.5 |
| | 4-24 | R-24 | 90 | 23 | 2.3 |
| | 4-25 | R-25 | 95 | 24 | 2.4 |

TABLE 8

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Example | 4-26 | R-26 | 95 | 22 | 2.3 |
| | 4-27 | R-27 | 95 | 25 | 2.5 |
| | 4-28 | R-28 | 95 | 24 | 2.3 |
| | 4-29 | R-29 | 90 | 22 | 2.3 |
| | 4-30 | R-30 | 90 | 22 | 2.2 |
| | 4-31 | R-31 | 95 | 25 | 2.4 |
| | 4-32 | R-32 | 90 | 23 | 2.3 |
| | 4-33 | R-33 | 95 | 22 | 2.3 |
| | 4-34 | R-34 | 95 | 24 | 2.5 |
| | 4-35 | R-35 | 95 | 23 | 2.3 |
| | 4-36 | R-36 | 95 | 25 | 2.5 |
| | 4-37 | R-37 | 95 | 22 | 2.3 |
| | 4-38 | R-38 | 90 | 25 | 2.2 |
| | 4-39 | R-39 | 95 | 24 | 2.4 |
| | 4-40 | R-40 | 90 | 25 | 2.3 |
| | 4-41 | R-41 | 95 | 23 | 2.2 |
| | 4-42 | R-42 | 95 | 22 | 2.4 |
| | 4-43 | R-43 | 90 | 25 | 2.3 |
| | 4-44 | R-44 | 95 | 24 | 2.5 |
| | 4-45 | R-45 | 95 | 22 | 2.4 |

TABLE 9

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Comparative | 3-1 | CR-1 | 95 | 26 | 2.7 |
| Example | 3-2 | CR-2 | 95 | 27 | 2.9 |
| | 3-3 | CR-3 | 95 | 29 | 2.9 |
| | 3-4 | CR-4 | 90 | 31 | 3.1 |
| | 3-5 | CR-5 | 95 | 26 | 2.6 |
| | 3-6 | CR-6 | 95 | 28 | 2.8 |
| | 3-7 | CR-7 | 90 | 27 | 2.9 |
| | 3-8 | CR-8 | 90 | 29 | 3.1 |
| | 3-9 | CR-9 | 90 | 28 | 2.9 |
| | 3-10 | CR-10 | 95 | 30 | 2.7 |
| | 3-11 | CR-11 | 95 | 27 | 2.6 |
| | 3-12 | CR-12 | 95 | 30 | 2.9 |
| | 3-13 | CR-13 | 90 | 29 | 2.9 |
| | 3-14 | CR-14 | 95 | 28 | 3.1 |
| | 3-15 | CR-15 | 95 | 29 | 2.9 |
| | 3-16 | CR-16 | 95 | 30 | 2.8 |
| | 3-17 | CR-17 | 95 | 31 | 2.9 |
| | 3-18 | CR-18 | 95 | 28 | 2.8 |
| | 3-19 | CR-19 | 90 | 29 | 3.0 |
| | 3-20 | CR-20 | 95 | 27 | 2.8 |
| | 3-21 | CR-21 | 95 | 29 | 2.9 |
| | 3-22 | CR-22 | 90 | 28 | 3.1 |
| | 3-23 | CR-23 | 90 | 30 | 2.8 |
| | 3-24 | CR-24 | 95 | 31 | 2.7 |
| | 3-25 | CR-25 | 95 | 28 | 2.9 |

From the results shown in Tables 7 to 9, it was verified that the chemically amplified resist composition containing the quencher of the invention has good sensitivity in both positive and negative tones and is excellent in CDU.

Japanese Patent Application No. 2024-091395 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt having the formula (1): 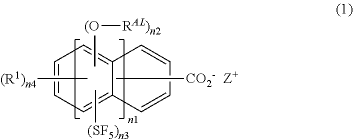
wherein n1 is 0 or 1, n2 is 1, 2, or 3, n3 is 1 or 2, n4 is 0, 1, or 2, meeting 2≤n2+n3+n4≤5 when n1 is 0, and 2≤n2+n3+n4≤7 when n1 is 1,
R^{AL} is an acid labile group, at least one —SFs group and at least one —O—R^{AL} are attached to mutually adjacent carbon atoms,
R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, two R¹'s may be identical or different and two R¹'s may bond together to form a ring with the carbon atoms to which they are attached, and
Z⁺ is an onium cation.

2. The onium salt of claim 1 having the formula (1A): 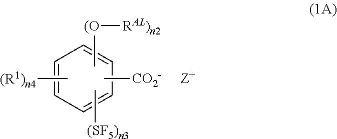
wherein n2 to n4, R¹, R^{AL}, and Z⁺ are as defined above.

3. The onium salt of claim 1 wherein the acid labile group is a group having the formula (AL-1) or (AL-2): 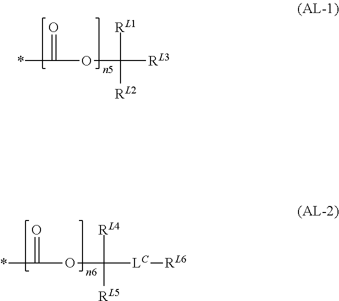
wherein n5 is 0 or 1, n6 is 0 or 1,
R^{L1}, R^{L2}, and R^{L3} are each independently a C₁-C₁₂ hydrocarbyl group, some —CH₂— in the hydrocarbyl group may be replaced by —O— or —S—, when the hydrocarbyl group contains an aromatic ring, some or all hydrogen atoms in the aromatic ring may be substituted with a halogen atom, a cyano group, a nitro group, a C₁-C₄ alkyl group which may contain a halogen atom, or a C₁-C₄ alkoxy group which may contain a halogen atom,
R^{L1} and R^{L2} may bond together to form a ring, some —CH₂— in the ring may be replaced by —O— or —S—,
R^{L4} and R^{L5} are each independently a hydrogen atom or a C₁-C₁₀ hydrocarbyl group, R^{L6} is a C₁-C₂₀ hydrocarbyl group, some —CH₂— in the hydrocarbyl group may be replaced by —O— or —S—, R¹⁵ and R^{L6} may bond together to form a C₃-C₂₀ heterocyclic group with the carbon atom and L^{C} to which they are attached, some —CH₂— in the heterocyclic group may be replaced by —O— or —S—,
L^{C} is —O— or —S—, and
* designates a point of attachment to the adjacent —O—.

4. The onium salt of claim 1, wherein Z⁺ is a sulfonium cation having the formula (Z-1), an iodonium cation having the formula (Z-2), or an ammonium cation having the formula (Z-3): 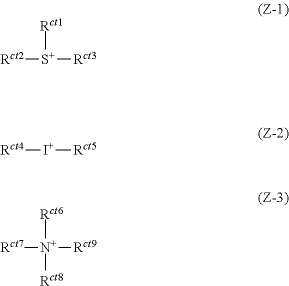
wherein R^{ct1} to R^{ct9} are each independently a halogen atom or a C₁-C₃₀ hydrocarbyl group which may contain a heteroatom, and R^{ct1} and R^{ct2} may bond together to form a ring with the sulfur atom to which they are attached, and any two of R^{ct6} to R^{ct9} may bond together to form a ring with the nitrogen atom to which they are attached.

5. The onium salt of claim 1, wherein Z⁺ is a sulfonium cation having the formula (Z-4): 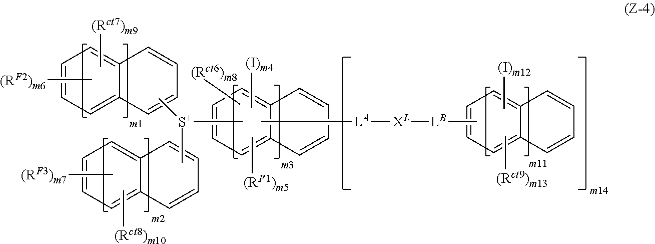
wherein m¹ is 0 or 1, m² is 0 or 1, m³ is 0 or 1, m⁴ is 0, 1, 2, 3, or 4, m⁵ is 0, 1, 2, 3, or 4, m⁶ is 0, 1, 2, 3, 4, 5, or 6, m⁷ is 0, 1, 2, 3, 4, 5, or 6, m⁸ is 0, 1, or 2, m⁹ is 0, 1, or 2, m¹⁰ is 0, 1, or 2, m¹¹ is 0 or 1, m¹² is 0, 1, 2, 3, or 4, m¹³ is 0, 1, or 2, m¹⁴ is 0, 1, or 2, meeting 0≤m⁶+m^{9≤4} when m¹ is 0, 0≤m⁶+m^{9≤6} when m¹ is 1, 0≤m⁷+m^{10≤4} when m² is 0, 0≤m⁷+m¹⁰≤6 when m² is 1, 1≤m⁴+m⁵+m⁸+m^{14≤4} when m³ is 0, 1≤m⁴+m⁵+m⁸+m^{14≤6} when m³ is 1, 0≤m¹²+m^{13≤4} when m¹¹ is 0, and 0≤m¹²+m^{13≤6} when m¹¹ is 1, and m⁴+m^{12≥1},
R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when m⁵ is 2 or more, a plurality of RFI's may be identical or different, when m⁶ is 2 or more, a plurality of R^{F2}'s may be identical or different, when m⁷ is 2 or more, a plurality of R^{F3}'s may be identical or different,
R^{ct6} to R^{ct9} are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m⁸ is 2, two R^{ct6}'s may be identical or different and two R^{ct6}'s may bond together to form a ring with the carbon atom to which they are attached, when m⁹ is 2, two R^{ct7}'s may be identical or different and two R^{ct7}'s may bond together to form a ring with the carbon atom to which they are attached, when m¹⁰ is 2, two R^{ct8}'s may be identical or different and two R^{ct8}'s may bond together to form a ring with the carbon atom to which they are attached, when m¹³ is 2, two R^{ct9}'s may be identical or different and two R^{ct9}'s may bond together to form a ring with the carbon atom to which they are attached,
the aromatic rings directly attached to S⁺ in the sulfonium cation may bond together to form a ring with S⁺,
L^{A} and L^{B} are each independently a single bond, an ether bond, an ester bond, an amide bond, a sulfonate ester bond, a sulfonamide bond, a carbonate bond, or a carbamate bond, and
X^{L} is a single bond or a C₁-C₄₀ hydrocarbylene group which may contain a heteroatom.

6. A quencher comprising the onium salt of claim 1.

7. A chemically amplified resist composition comprising the quencher of claim 6.

8. The chemically amplified resist composition of claim 7, further comprising a base polymer.

9. The chemically amplified resist composition of claim 8, wherein the base polymer contains at least one repeat unit selected from a repeat unit having the formula (a1) and a repeat unit having the formula (a2): 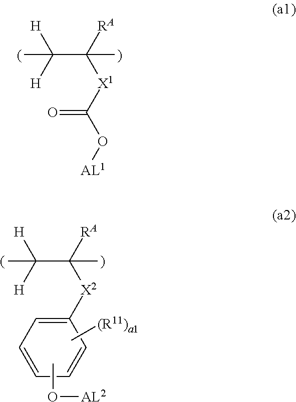
wherein R^{A}'s are each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom in the backbone,
R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2, 3, or 4, R¹¹'s may be identical or different,
AL¹ and AL² are each independently an acid labile group, and
a1 is 0, 1, 2, 3, or 4.

10. The chemically amplified resist composition of claim 8, wherein the base polymer contains a repeat unit having the formula (a3): 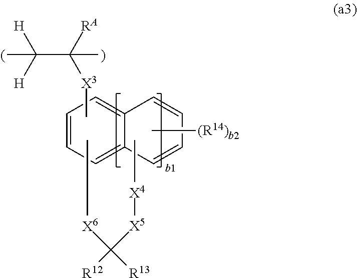
wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom in the backbone,
X⁴ is a single bond, a C₁-C₄ aliphatic hydrocarbylene group, a carbonyl group, a sulfonyl group, or a group obtained by combining these groups,
X⁵ and X⁶ are each independently an oxygen atom or a sulfur atom, X⁴ and X⁶ are attached to the adjacent carbon atoms on the aromatic rings,
R¹² and R¹³ are each independently a hydrogen atom or a C₁-C₂₀ hydrocarbyl group which may contain a heteroatom, and R¹² and R¹³ may bond together to form a ring with the carbon atom to which they are attached,
R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A}) (R^{14B}), R^{14A} and R^{14B} are each independently a hydrogen atom or a C₁-C₆ hydrocarbyl group, when b2 is 2 or more, a plurality of R¹⁴'s may be identical or different, and a plurality of R¹⁴'s may bond together to form a ring with the carbon atom on the aromatic ring to which they are attached.

11. The chemically amplified resist composition of claim 8, wherein the base polymer does not contain an acid labile group.

12. The chemically amplified resist composition of claim 8, wherein the base polymer contains at least one repeat unit selected from a repeat unit having the formula (b1) and a repeat unit having the formula (b2): 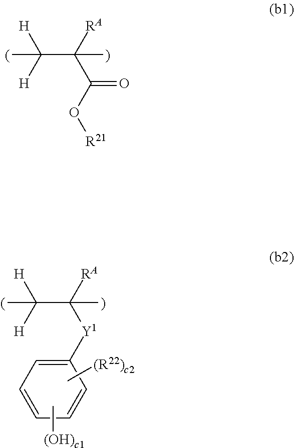
wherein R^{A}'s are each independently a hydrogen atom, a fluorine atom, a methyl group, or a trifluoromethyl group,
Y¹ is a single bond or *—C(═O)—O—, * designates a point of attachment to the carbon atom in the backbone,
R²¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2, 3, or 4, a plurality of R²²'s may be identical or different,
c1 is 1, 2, 3 or 4, and c2 is 0, 1, 2, 3, or 4, meeting 1≤c1+c2≤5.

13. The chemically amplified resist composition of claim 8, wherein the base polymer contains at least one repeat unit selected from a repeat unit having the formula (c1), a repeat unit having the formula (c2), a repeat unit having the formula (c3), a repeat unit having the formula (c4), and a repeat unit having the formula (c5): 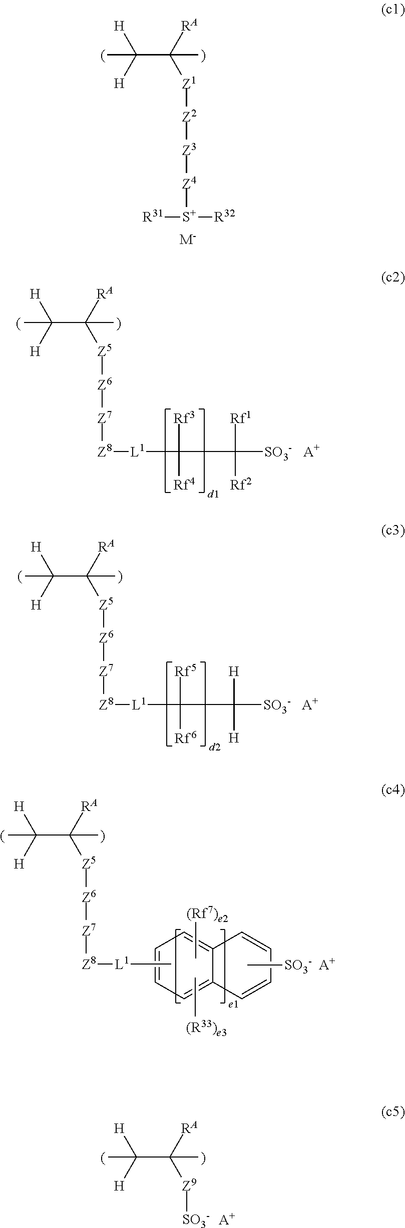
wherein d1 and d2 are each independently 0, 1, 2, or 3, e1 is 0 or 1, e2 is 0, 1, 2, 3, or 4, and e3 is 0, 1, 2, 3, or 4, meeting 0≤e2+e3≤4 when e1 is 0, and 0≤e2+e3≤6 when e1 is 1,
R^{A}'s are each independently a hydrogen atom, a fluorine atom, a methyl group, or a trifluoromethyl group,
Z¹ is a single bond or a phenylene group which may have a substituent,
Z² is a single bond, **—C(═O)—O—Z²¹—, **—C(═O)—NH—Z²¹—, or **—O—Z²¹—, Z²¹ is a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z³ is a single bond, an ether bond, an ester bond, an amide bond, a sulfonate ester bond, a sulfonamide bond, a carbonate bond, or a carbamate bond,
Z⁴ is a single bond, or a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z⁵'s are each independently a single bond, a phenylene group which may have a substituent, a naphthylene group, or *—C(═O)—O—Z⁵¹—, Z⁵¹ is a C₁-C₁₀ aliphatic hydrocarbylene group, a phenylene group, or a naphthylene group, and the aliphatic hydrocarbylene group may contain a halogen atom, a hydroxy group, an ether bond, an ester bond, or a lactone ring,
Z⁶ is a single bond, an ether bond, an ester bond, an amide bond, a sulfonate ester bond, a sulfonamide bond, a carbonate bond, or a carbamate bond,
Z⁷'s are each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
Z⁸'s are each independently a single bond, ****—Z⁸¹—C(═O)—O—, ****—C(═O)—NH—Z⁸¹—, or ****—O—Z⁸¹—, Z⁸¹ is a C₁-C₂₀ hydrocarbylene group which may contain a heteroatom,
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—N(H)—Z⁹¹—, or *—O—Z⁹¹—, Z⁹¹ is a C₁-C₆ aliphatic hydrocarbylene group, a phenylene group, a fluorinated phenylene group, or a phenylene group substituted with a trifluoromethyl group, and may contain a carbonyl group, an ester bond, an ether bond, or a hydroxy group,
designates a point of attachment to the carbon atom in the backbone, ** designates a point of attachment to Z¹, *** designates a point of attachment to Z⁶, and **** designates a point of attachment to Z⁷,
L¹ is a single bond, an ether bond, an ester bond, a carbonyl group, a sulfonate ester bond, a sulfonamide bond, a carbonate bond, or a carbamate bond,
Rf¹ and Rf² are each independently a fluorine atom or a C₁-C₆ fluorinated saturated hydrocarbyl group,
Rf³ and Rf⁴ are each independently a hydrogen atom, a fluorine atom, or a C₁-C₆ fluorinated saturated hydrocarbyl group,
Rf⁵ and Rf⁶ are each independently a hydrogen atom, a fluorine atom, or a C₁-C₆ fluorinated saturated hydrocarbyl group, excluding that all Rf⁵ and Rf⁶ are a hydrogen atom at the same time,
Rf⁷ is a fluorine atom, a C₁-C₆ fluorinated alkyl group, a C₁-C₆ fluorinated alkoxy group, or a C₁-C₆ fluorinated alkylthio group,
R³¹ and R³² are each independently a C₁-C₂₀ hydrocarbyl group which may contain a heteroatom, and R³¹ and R³² may bond together to form a ring with the sulfur atom to which they are attached,
R³³ is a halogen atom other than a fluorine atom, or a C₁-C₂₀ hydrocarbyl group which may contain a heteroatom, when e3 is 2, 3, or 4, a plurality of R³³'s may be identical or different, and a plurality of R³³'s may bond together to form a ring with the carbon atom to which they are attached,
M⁻ is a non-nucleophilic counter ion, and
A⁺ is an onium cation.

14. The chemically amplified resist composition of claim 7, further comprising an organic solvent.

15. The chemically amplified resist composition of claim 7, further comprising an acid generator that generates an acid having a pKa≤−2.0.

16. The chemically amplified resist composition of claim 7, further comprising a quencher other than the quencher.

17. The chemically amplified resist composition of claim 7, further comprising a surfactant.

18. A pattern forming process comprising the steps of forming a resist film on a substrate using the chemically amplified resist composition of claim 7, exposing the resist film to high-energy radiation, and developing the exposed resist film using a developer.

19. The pattern forming process of claim 18, wherein the high-energy radiation is KrF excimer laser light, ArF excimer laser light, an electron beam, or an extreme ultraviolet ray with a wavelength of 3 to 15 nm.
